(19) United States
(12) Patent Application Publication
(10) Pub. No.: US 2003/0078251 A1
(43) Pub. Date: Apr. 24, 2003

(54) Benzoxazepinones and their use as squalene synthase inhibitors

(76) Inventors: Masakuni Kori, Nishi-ku Kobe-shi Hyogo (JP); Takashi Miki, Osaka-shi Osaka (JP); Tomoyuki Nishimoto, Ikeda-shi Osaka (JP); Ryuichi Tozawa, Tsukuba-shi Ibaraki (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/203,524

(22) PCT Filed: Jun. 22, 2001
(86) PCT No.: PCT/JP01/05347

(30) Foreign Application Priority Data
Jun. 23, 2000 (JP) 2000-190253

(51) Int. Cl. A61K 31/553; C07D 281/02

(52) U.S. Cl. 514/211.05; 540/490

(57) ABSTRACT

There is disclosed a compound represented by the formula [I]:

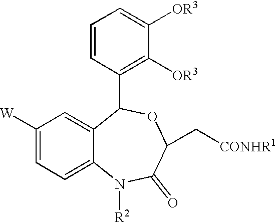

wherein R¹ is optionally substituted 1-carboxyethyl group, optionally substituted alkyl-sulfonyl group, optionally substituted (carboxy-cycloalkyl)-alkyl group, —X¹—X²—Ar—X³—X⁴—COOH (wherein X¹ and X⁴ are a bond or alkylene group, X² and X³ are a bond, —O—, —S—, Ar is divalent aromatic group etc.), R² is alkyl group optionally substituted with alkanoyloxy group and/or hydroxy group, R³ is alkyl group, and W is halogen atom, etc., or a salt thereof. The compound has the cholesterol lowering activity and the triglyceride lowering activity and is useful for preventing and/or treating hyperlipidemia.

### TECHNICAL FIELD

The present invention relates to a novel benzoxazepine compound which is useful for preventing and/or treating hyperlipidemia and has the cholesterol lowering activity and the triglyceride lowering activity.

### BACKGROUND OF THE INVENTION

An abnormal increase in the concentration of serum lipid is called hyperlipidemia or hyperlipemia. There are many serum lipids, that is, cholesterol (cholesterol ester, free cholesterol), phospholipid (lecithin, sphingomyelin, etc.), triglycerides (neutral lipid), free fatty acid and other sterols. In particular, a clinical problem is an increase in cholesterol or triglyceride (COMMON DISEASE SERIES No. 19, Hyperlipidemia, ed. by Haruo Nakamura, published on Oct. 10, 1991, Nankodo).

Examples of a drug for lowering a cholesterol value in blood include drugs which trap bile acid and inhibits its absorption such as cholestyramine and colestipol (for example, U.S. Pat. No. 4,027,009), drugs which inhibit acyl coenzyme A cholesterol acyl transferase (ACAT) such as melinamide (French Patent No. 1476569) and inhibit absorption of cholesterol into an intestinal tract, and drugs which inhibit biosynthesis of cholesterol. As the cholesterol biosynthesis inhibiting drug, there are in particular lovaststin (U.S. Pat. No. 4,231,938), simvastatin (U.S. Pet. No. 4,444,784) and pravastatin (U.S. Pat. No. 4,346,227) which inhibits 3-hydroxy-3-methylglutaryl coenzyme A (HMG-CoA) reductase serve as a drug.

In addition, as a triglyceride lowering agent, fibric acid type compound, such a chlofibrate (British Patent No.860303) and fenofibrate (German Patent No. 2250327) serve as a drug.

On the other hand, compounds having the cholesterol biosynthesis inhibiting activity by inhibition of a squalene synthase are disclosed in Journal of Medicinal Chemistry, vol. 51, No. 10, pp. 1869-1871, 1988, JP-A H1(1989)-213288, JP-A H2(1990)-101088, JP-A H2(1990)-235820, JP-A H2(1990)-235821, JP-A H3(1991)-20226, JP-A H3(1991)-68591, JP-A H3(1991)-148288, as well as U.S. Pat. No. 5,019,390, U.S.P. No. 5,135,935, U.S.P. No. 5,726,306, U.S.P. No. 5,698,691, EP 0645377, WO9215579, WO9309115, and WO9710224.

### OBJECTS OF THE INVENTION

Suitable control of the serum lipid concentration is extremely important for preventing or treating diseases associated with atherosclerosis, a representative of which are ischemic heart failure and cerebral infarction. In addition, hypertriglyceridemia is considered to be complicated with pancreatic disorder. Since when HMG-COA reductase is inhibited by a HMG-COA reductase inhibitor, biosynthesis of other components necessary for the living body such as ubiquinone, dolichol and heme A in addition to biosynthesis of cholesterol is inhibited, side effects derived therefrom are worried about. In addition, the use of a triglyceride lowering agent and a statin type compound at the same time is prohibited due to hepatic toxicity. On the other hand, a squalene synthase is an enzyme involved in an essential stage in the cholesterol biosynthetic pathway. This enzyme is an enzyme which catalyzes reductive dimerization of 2 molecules of farnesyl pyrophosphate to form squalene.

Under the circumstances, an object of the present invention is to provide a compound which is safer, has the stronger lipid lowering activity such as the squalene synthase inhibiting activity (cholesterol lowering activity) and the triglyceride lowering activity, and is useful as a drug for preventing or treating hyperlipidemia.

### SUMMARY OF THE INVENTION

The present inventors studied intensively and, as a result, we first synthesized a 4,1-benzoxazepine compound having the characteristic of the chemical structure having specific substituents at 1-position, 3-position, 5-position and 7-position and found that this compound has unexpectedly the drug activities such as the excellent lipid lowering activity based on the unique chemical structure, which resulted in completion of the present invention.

That is, the present invention relates to:

1. A compound represented by the formula [I]:

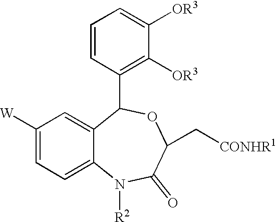

wherein R¹ is optionally substituted 1-carboxyethyl group, optionally substituted carboxy-C₃₋₆ straight alkyl group, optionally substituted C₃₋₆ straight alkyl-sulfonyl group, optionally substituted (carboxy-C₅₋₇ cycloalkyl)-C₁₋₃ alkyl group, or a group represented by the formula: —X¹—X²—Ar—X³—X⁴—COOH (wherein each of X¹ and X⁴ is a bond or optionally substituted C₁₋₄ alkylene group, each of X² and X³ is a bond, —O— or —S—, and Ar is optionally substituted bivalent aromatic group, provided that, when X¹ is a bond, X² is a bond and, when X⁴ is a bond, X³ is a bond), R² is C₃₋₆ alkyl group optionally substituted with alkanoyloxy group and/or hydroxy group, R³ is lower alkyl group, and W is halogen atom, provided that, when R¹ is optionally substituted 1-carboxyethyl group, optionally substituted C₃₋₆ straight alkyl group, 4-carboxycyclohexylmethyl group or 4-carboxymethylphenyl group, R² is C₃₋₆ alkyl group having alkanoyloxy group and/or hydroxy group, or a salt thereof;

2. The compound according to the above 1, wherein R¹ is 3-carboxypropyl group, 1-carboxyethyl group, optionally substituted C₃₋₆ straight alkyl-sulfonyl group, optinally substituted (carboxy-C₅₋₇ cycloalkyl)-C₁₋₃ alkyl group, optionally substituted (carboxyfuryl)-alkyl group, optionally substituted carboxy-C₆₋₁₀ aryl group, (carboxy-C₂₋₃ alkyl)-C₆₋₁₀ aryl group or (carboxy-C₁₋₃ alkyl) —C₇₋₁₄ aralkyl group;

3. The compound according to the above 1, wherein R¹ is optionally substituted (carboxy-C₁₋₄ alkyl)-C₆₋₁₀ aryl group;

4. The compound according to the above 1, wherein R¹ is optionally substituted (carboxy-C₂₋₃ alkyl)-C₆₋₁₀ aryl group;

5. The compound according to the above 1, wherein R¹ is optionally substituted (carboxy-C₂₋₃ alkyl)phenyl group;

6. The compound according to the above 1, wherein R¹ is optionally substituted (carboxyfuryl)-alkyl group;

7. The compound according to the above 1, wherein R² is C₃₋₆ alkyl group having alkanoyloxy group and/or hydroxy group;

8. The compound according to the above 1, wherein R² is C₃₋₆ alkyl group optionally having 1 to 3 substituents selected from hydroxy group, acetoxy, propionyloxy, t-butoxycarbonyloxy and palmitoyloxy;

9. The compound according to the above 1, wherein R² is 2,2-dimethylpropyl, 3-hydroxy-2,2-dimethylpropyl or 3-acetoxy-2,2-dimethylpropyl;

10. The compound according to the above 1, wherein R³ is methyl group;

11. The compound according to the above 1, wherein W is chlorine atom;

12. The compound according to the above 1, wherein a 3-position is R-configuration and a 5-position is S-configuration;

13. The compound according to the above 1, which is:

(3R,5S)-N-propanesulfonyl-7-chloro-5-(2,3-dimethoxyphenyl)-1-(3-hydroxy-2,2-dimethylpropyl)-1,2,3,5-tetrahydro-4,1-benzoxazepine-3-acetamide, or a salt thereof

(2R)-2-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminopropionic acid, or a salt thereof,

3-[3-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminophenyl]propionic acid, or a salt thereof, or

4-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminobutanoic acid, or a salt thereof;

14. The compound according to the above 1, which is:

trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl-1-cyclohexanecarboxylic acid, or a salt thereof,

trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylphenyl]propionic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]propionic acid, or a salt thereof,

4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxylphenyl]butanoic acid, or a salt thereof,

5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid, or a salt thereof, or

5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid, or a salt thereof;

15. The compound according to the above 1, which is:

2-[2-[[[(3R,5S)-7-chloro-5-(2,3-dimethoxyphenyl)-1-(3-hydroxypropyl-2,2-dimethylpropyl)-2-oxo-1,2,3,5-tetrahydro-4,1-benzoxazepin-3-yl]acetyl]amino]ethyl]furan-3-carboxylic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid, or a salt thereof, or

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid, or a salt thereof;

16. A prodrug of a compound represented by the formula [I]

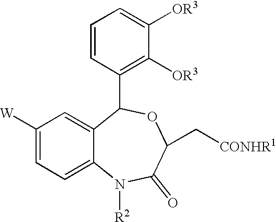

wherein each symbol is as defined in claim, or a salt thereof;

17. A process for producing a compound represented by the formula [I]:

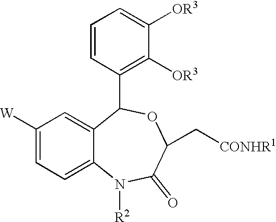

wherein each symbol is as defined in claim 1, or a salt thereof,

which comprises reacting a compound represented by the formula [II]:

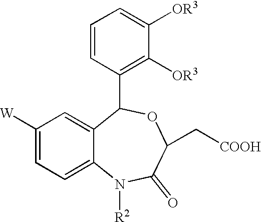

wherein each symbol is as defined in claim 1, or a salt thereof or a reactive derivative of the carboxyl group, with a compound represented by the formula:

H₂N—R¹

wherein each symbol is as defined in claim 1, or a salt thereof.

18. A pharmaceutical composition comprises a compound represented by the formula [I]:

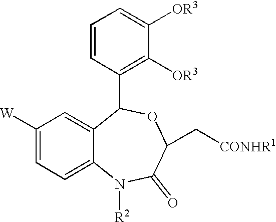

wherein each symbol is as defined in claim 1, a salt thereof or a prodrug thereof;

19. The pharmaceutical composition according to the above 18, which is a squalene synthase inhibitor;

20. The pharmaceutical composition according to the above 18, which is a triglyceride lowering agent;

21. The pharmaceutical composition according to the above 18, which is a lipid lowering agent;

22. The pharmaceutical composition according to the above 18, which is an agent for preventing and/or treating hyperlipidemia;

23. The pharmaceutical composition according to the above 18, which is a high-density lipoproetin cholesterol increasing agent;

24. A method for inhibiting squalene synthase in a mammal in need thereof which comprises administering an effective amount of the compound according to the above 1, or a salt or a prodrug thereof to said mammal;

25. A method for lowering triglycerides in a mammal in need thereof which comprises administering an effective amount of the compound according to the above 1, or a salt or a prodrug thereof to said mammal;

26. A method for lowering lipid in a mammal in need thereof which comprises administering an effective amount of the compound according to the above 1, or a salt or a prodrug thereof to said mammal;

27. A method for preventing and/or treating hyperlipidemia of a mammal in need thereof which comprises administering an effective amount of the compound according to the above 1, or a salt or a prodrug thereof to said mammal;

28. A method for increasing high-density lipoprotein-cholesterol in a mammal in need thereof which comprises administering an effective amount of the compound according to the above 1, or a salt or a prodrug thereof to said mammal;

29. Use of the compound according to the above 1, or a salt or a prodrug thereof for manufacturing a squalene synthase inhibior;

30. Use of the compound according to the above 1, or a salt or a prodrug thereof for manufacturing a triglyceride lowering agent;

31. Use of the compound according to the above 1, or a salt or a prodrug thereof for manufacturing a lipid lowering agent;

32. Use of the compound according to the above 1, or a salt or a prodrug thereof for manufacturing an agent for preventing and/or treating hyperlipidemia; and

33. Use of the compound according to the above 1, or a salt or a prodrug thereof for manufacturing a high-density lipoprotein-cholesterol increasing agent.

### DETAILED DESCRIPTION OF THE INVENTION

In the above formula, R¹ is optionally substituted 1-carboxyethyl group, optionally substituted carboxy-C₃₋₆ straight alkyl group, optionally substituted C₃₋₆ straight alkyl-sulfonyl group, optionally substituted (carboxy-C₁₋₇ cycloalkyl)-C₁₋₃ alkyl group, or a group represented by the formula: —X¹—X²—Ar—X³—X⁴—COOH (wherein each of X¹ and X⁴ is a bond or optionally substituted C₁₋₄ alkylene group, each of X² and X³ is a bond, —O— or —S—, and Ar is optionally substituted bivalent aromatic group, provided that, when X¹ is a bond, X² is a bond and, when X⁴ is a bond, X³ is a bond).

Examples of C₃₋₆ straight alkyl group in the optionally substituted carboxy-C₃₋₆ straight alkyl group include n-propyl, n-butyl, n-pentyl, n-hexyl. Among them, n-propyl and n-butyl are preferable, with n-propyl being more preferable.

Examples of C₃₋₆ straight alkyl group in the optionally substituted C₃₋₆ straight alkyl-sulfonyl group represented by R¹ include n-propyl, n-butyl, n-pentyl and n-hexyl. Among them, n-propyl and n-butyl are preferable, and n-propyl is more preferable.

Examples of C₅₋₇ cycloalkyl group in the optionally substituted (carboxy-C₅₋₇ cycloalkyl)-C₁₋₃ alkyl group optionally represented by R¹ include cyclopentyl, cyclohexyl and cycloheptyl. Among them, cyclopentyl and cyclohexyl are preferable, and cyclohexyl is more preferable.

Examples of C₁₋₃ alkyl group in the optionally substituted (carboxy-C₅₋₇ cycloalkyl)-C₁₋₃ alkyl group optionally represented by R¹ include methyl, ethyl, n-propyl and isopropyl. Among them, methyl and ethyl are preferred, and methyl is more preferable.

Examples of “C₁₋₄ alkylene group” in the “optionally substituted C₁₋₄ alkylene group” represented by X¹ and X⁴ of the group represented by the formula X¹—X²—Ar—X³—X⁴—COOH of R¹ include methylene, dimethylene, trimethylene, tetramethylene, etc., and C₁₋₃ alkylene group is preferable. In particular, a straight one is preferable.

Examples of the “bivalent aromatic group” in the “optionally substituted bivalent aromatic group” represented by Ar include bivalent aromatic hydrocarbon group, bivalent aromatic heterocyclic group, etc.

Hereupon, as the bivalent aromatic hydrocarbon group, for example, there is a group formed by removing any one of hydrogen atoms from C₆₋₁₀ aryl group (e.g., phenyl, naphthyl, etc.) etc., and, as the bivalent aromatic hydrocarbon group, phenylene is preferable.

As the bivalent aromatic heterocyclic group, for example, there is a group formed by removing any one of hydrogen atoms from an aromatic heterocyclic group containing as the ring-constituent atoms (ring atoms) at least one (preferably 1 to 4, more preferably 1 to 2) hetero atom selected from one to three (preferably one or two) kinds of hetero atoms selected from oxygen atom, sulfur atom and nitrogen atom, etc.

Hereupon, examples of the aromatic heterocyclic group include 5- or 6-membered atomatic monocyclic heterocyclic groups such as furyl, thienyl, pyrrolyl, oxazolyl, isozazolyl, thiazolyl, isothiazolyl, imidazolyl, pyrazolyl, 1,2,3-oxadiazolyl, 1,2,4-oxadiazolyl, 1,3,4-oxadizaolyl, frazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl, etc. (preferably, furyl, thienyl, pyrrolyl, imidazolyl, thiazolyl, pyridyl, etc.); 8- to 12-membered aromatic fused heterocyclic groups such as benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazlyl, benzopyranyl, 1,2-benzothiazolyl, 1H-benzotriazolyl, quinolyl, isoquinolyl, cinnolinyl, quinazolinyl, quinoxalinyl, phthalazinyl, nephthyridinyl, purinyl, pteridinyl, carbazolyl, α-carbolinyl, β-carbolinyl, y-carbolinyl, acridinyl, phenoxazinyl, phenothiazinyl, phenazinyl, phenoxathiinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-1]pyridazinyl, imidazo[1,2-a]pyrimidinyl, 1,2,4-triazolo[4,3-a]pyridyl, 1,2,4-triazolo[4,3-1]pyridazinyl, etc.; (preferably, a heterocyclic ring composed by the above-mentioned 5- or 6-membered aromatic monocyclic heterocyclic group fused with benzene ring, or a heterocyclic ring composed by the same or different two above-mentioned 5- or 6-membered aromatic monocyclic heterocyclic groups fused with benzene ring, more preferably, a heterocyclic ring composed by the above-mentioned 5- or 6-membered aromatic monocyclic heterocyclic group) and the like.

Examples of the substituent of “C₁₋₄ alkylene group” of the “optionally substituted C₁₋₄ alkylene group” represented by X¹ and X⁴ and the “bivalent aromatic group” of the “optionally substituted bivalent aromatic group” represented by Ar include (i) carboxyl group optionally esterified with C₁₋₆ alkyl group or C₆₋₁₀ aryl-C₁₋₄ alkyl group (for example, methyl, ethyl, propyl, isopropyl, butyl, t-butyl, phenyl, benzyl and the like), (ii) phosphoric acid group optionally mono- or di-substituted with C₁₋₆ alkyl (for example, methyl, ethyl, n-propyl, isopropyl, n-butyl, isobutyl, n-pentyl, isopentyl, neopentyl, hexyl and the like) or C₂₋₇ alkanoyloxy-C₁₋₆ alkyl such as acetoxymethyl and pivaloyloxymethyl, (iii) sulfonic acid group, (iv) sulfonamide group optionally substituted with C₁₋₆ alkyl group or C₆₋₁₀ aryl-C₁₋₄ alkyl group (for example, methyl, ethyl, propyl, isopropyl, butyl, t-butyl, benzyl and the like), (v) hydroxy group and sulfhydryl group optionally alkylated with C₁₋₃ alkyl group (for example, methyl, ethyl, propyl and the like), (vi) carbamoyl group, (vii) phenyl group optionally substituted with 1 to 5 substituents [for example, hydroxy group, chlorine, fluorine, aminosulfonyl group, amino group optionally substituted with C₁₋₃ alkyl group (for example, methyl, ethyl, propyl and the like)], which may be attached via O or S, (viii) amino group optionally mono- or di-substituted with C₁₋₃ alkyl group (for example, methyl, ethyl, propyl and the like), (ix) cyclic amino group (for example, 5-6 membered cyclic amino group optionally containing oxygen atom or sulfur atom as a cyclic constituent atom in addition to nitrogen atom, such as cyclic amino group derived (by removing one hydrogen atom) from cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline, and phthalimido and the like) optionally substituted with 1 to 3 of C₁₋₃ alkyl (for example, methyl, ethyl and the like), benzyl, phenyl and the like, (x) 5-6 membered aromatic heterocyclic group containing 1 to 4 hetero atoms selected from N, O and S (for example, pyridyl, imidazolyl, indolyl, tetrazolyl and the like), which may be attached via O or S, (xi) halogen atom (for example, chlorine, fluorine, bromine, iodine, etc.), (xii) C₁₋₄ alkyl group (for example, methyl, ethyl, propyl, isopropyl, butyl, t-butyl, etc.), C₁₋₄ alkoxy group (for example, methoxy, ethoxy, propoxy, isopropoxy, butoxy, t-butoxy, etc.) and C₁₋₄ alkylthio (for example, methylthio, ethylthio, propylthio, isopropylthio, butylthio, t-butylthio, etc.), each of which may be substituted with a substituent selected from C₁₋₄ alkoxy group, C₁₋₄ alkylthio group, carboxyl and phenyl, (xiii) C₅₋₇ cycloalkyl group (for example, cyclopentyl, cyclohexyl, cycloheptyl, etc.), and (xiv) C1-7 alkanoyloxy (for example, formyloxy, acetoxy, propionyloxy, butyryloxy, t-butoxycarbonyloxy, isobutyryloxy, valeryloxy, pivaloyloxy, etc.). The number of these substituents can be 1 to 6, preferably 1 to 3 at any possible positions. In addition, two substituents can be linked to each other to form C₃₋₆ alkylene, C₃₋₆ alkyleneoxy, C₃₋₆ alkylenedioxy or the like. For example, when two adjacent substituents on phenyl group are linked to each other, they form tetrahydronaphthalene group.

Specific examples of a group represented by the formula —X¹—X²—Ar—X³—X⁴—COOH as R¹ include optionally substituted (carboxy-heteroaryl)-C₁₋₄ alkyl group [preferably, optionally substituted (carboxy-furyl)-C₁₋₄ alkyl group], optionally substituted (carboxy-C₆₋₁₀ aryl)-C₁₋₄ alkyl group, optionally substituted carboxy-heteroaryl group, optionally substituted carboxy-C₆₋₁₀ aryl group, optionally substituted (carboxy-C₁₋₄ alkyl)-heteroaryl group, optionally substituted (carboxy-C₁₋₄ alkyl)-C₆₋₁₀ aryl group [preferably, (carboxy-C₂₋₃ alkyl)-C₆₋₁₀ aryl group], optionally substituted (carboxy-C₁₋₄ alkyl)-heteroaryl-C₁₋₄ alkyl group, optionally substituted (carboxy-C₁₋₄ alkyl)-C₇₋₁₄ aralkyl group [preferably, optionally substituted (carboxy-C₁₋₃ alkyl)-C₇₋₁₄ aralkyl group], optionally substituted (carboxy-C₁₋₄ alkoxy)-C₆₋₁₀ aryl group, optionally substituted (carboxy-C₁₋₄ alkoxy)-C₆₋₁₀ aryl-C₁₋₄ alkyl group, optionally substituted (carboxy-C₁₋₄ alkyl)-C₆₋₁₀ aryloxy-C₁₋₄ alkyl group, optionally substituted (carboxy-C₆₋₁₀ aryloxy)-C₁₋₄ alkyl group, optionally substituted (carboxy-C₁₋₄ alkylthio)-heteroaryl group, and the like.

Hereupon, examples of the heteroaryl include the same group as that exemplified with respect to the above “aromatic heterocyclic group” and the heteroaryl may have the same substituent as that of the above “aromatic heterocyclic group”. In addition, examples of C₆₋₁₀ aryl include phenyl, naphthyl and azulenyl with phenyl being preferable. The C₆₋₁₀ aryl may have the same substituent as that of the above “aromatic heterocyclic group”.

Examples of the “alkyl group” of the optionally substituted (carboxyfuryl)-C₁₋₄ alkyl represented by R¹ include C₁₋₄ straight or branched alkyl such as methyl, ethyl, n-propyl, isopropyl, n-butyl, isobutyl, 1,1-dimethylethyl, etc. Among them, preferred are C₁₋₄ alkyl group such as methyl, ethyl, n-propyl, isopropyl, n-butyl, etc. with methyl, ethyl and n-propyl being more preferable. Examples of the carboxyfuryl group include 3-carboxy-2-furyl, 4-carboxy-2-furyl, 2-carboxy-3-furyl, 2-carboxy-5-furyl, etc. Among them, preferred are 3-carboxy-2-furyl and 4-carboxy-2-furyl, with 3-carboxy-2-furyl being more preferable.

Examples of C₂₋₃ alkyl of the optionally substituted (carboxy-C₂₋₃ alkyl)-C₆₋₁₀ aryl group represented by R¹ include ethyl, n-propyl and isopropyl, with ethyl and n-propyl are preferable. As the C₆₋₁₀ aryl group, for example, there are phenyl, naphthyl and azulenyl, with phenyl being preferable.

Examples of C₁₋₃ alkyl of the optionally substituted (carboxy-C₁₋₃ alkyl)-C₇₋₁₄ aralkyl represented by R¹ include methyl, ethyl, n-propyl and isopropyl, with methyl and ethyl being preferable, and ethyl being particularly preferable. Examples of the C₇₋₁₄ aralkyl group include phenylmethyl, 1-phenylethyl, 2-phenylethyl, 3-phenylpropyl, 2-phenylpropyl, 4-phenylbutyl, (1-naphthyl)methyl, (2-naphthyl)methyl, 1-(1-naphthyl)ethyl, 1-(2-naphthyl)ethyl, 3-(1-naphthyl)propyl, 4-(1-naphthyl)butyl, 4-(2-naphthyl)butyl, etc. Phenylmethyl, 1-phenylethyl, 3-phenylpropyl, (1-naphthyl)methyl, (2-naphthyl)methyl, (1-naphthyl) ethyl and (2-naphthyl) ethyl are preferable, with phenylmethyl and 2-phenylethyl being particularly preferable.

When each group represented by R¹ has a substituent, examples thereof include the same substituent as that exemplified with respect to the “bivalent aromatic group” of “optionally subsituted bivalent aromatic group” represented by Ar. The number of such substituents can be 1 to 6, preferably 1 to 3 at any possible positions. In each group represented by R¹, preferably, the carboxy moiety is unsubstituted. However, any moiety other than carboxyl can be substituted at any possible positions.

Preferably, R¹ is 3-carboxypropyl group, 1-carboxyethyl group, optionally substituted C₃₋₆ straight alkyl-sulfonyl group, optionally substituted (carboxy-C₅₋₇ cycloalkyl)-C₁₋₃ alkyl group, optionally substituted (carboxyfuryl)-alkyl group, optionally substituted carboxy-C₆₋₁₀ aryl group, optionally substituted (carboxy-C₁₋₄ alkyl) —C₆₋₁₀ aryl group [preferably (carboxy-C₂₋₃ alkyl) —C₆₋₁₀ aryl group] or optionally substituted (carboxy-C₁₋₃ alkyl)—C₇₋₁₄ aralkyl group, and the like. More preferably, R¹ is optionally substituted (carboxy-C₁₋₄ alkyl)-C₆₋₁₀ aryl group, with optionally substituted (carboxy-C₂₋₃ alkyl)-C₆₋₁₀ aryl group being particularly preferable. Among them, optionally substituted (carboxy-C₂₋₃ alkyl)-C₆₋₁₀ aryl is particularly preferable.

Examples of C₃₋₆ alkyl group in the C₃₋₆ alkyl group optionally substituted with alkanoyloxy group or hydroxy group represented by R² include n-propyl, isopropyl, 1,1-dimethylethyl, n-butyl, isobutyl, n-pentyl, 2,2-dimethylpropyl, isopentyl, n-hexyl and isohexyl and the like. Among them, isopropyl, 1,1-dimethylethyl, n-butyl, isobutyl, 2,2-dimethylpropyl and isohexyl are preferable, with 2,2-dimethylpropyl being particularly preferable.

Examples of alkanoyloxy group in the C₃₋₆ alkyl group optionally substituted with alkanoyloxy group or hydroxy group represented by R² include C₁₋₂₀ alkanoyloxy group such as formyloxy, acetoxy, propionyloxy, butyryloxy, t-butoxycarbonyloxy, isobutyryloxy valeryloxy, pivaloyloxy, lauryloxy, palmitoyloxy, stearoyloxy (preferably, C₁₋₇ alkanoyloxy) and the like. Among them, acetoxy, propionyloxy, t-butoxycarbonyloxy, and palmitoyloxy are preferable, and acetoxy is particularly preferable. The number of alkanoyloxy group or hydroxy group can be 1 to 3 at any possible positions.

Preferable examples of C₃₋₆ alkyl group optionally substituted with alkanoyloxy group or hydroxy group represented by R² include 2,2-dimethylpropyl, 3-hydroxy-2,2-dimethylpropyl, 3-hydroxy-2-hydroxymethyl-2-methylpropyl, 3-acetoxy-2,2-dimethylpropyl, 3-acetoxy-2-hydroxymethyl-2-methylpropyl and 3-acetoxy-2-acetoxymethyl-2-methylpropyl. Among them, 2,2-dimethylpropyl, 3-hydroxy-2,2-dimethylpropyl and 3-acetoxy-2,2-dimethylpropyl are particularly preferable.

Preferably, R² is C₃₋₆ alkyl group having alkanoyloxy group and/or hydroxy group.

Examples of lower alkyl group represented by R³ include C₁₋₆ alkyl group such as methyl, ethyl, n-propyl, isopropyl, n-butyl, t-butyl, pentyl and hexyl. Inter alia, C₁₋₃ alkyl group is preferable. As R³, in particular, methyl group is preferable from a pharmacological viewpoint.

Examples of halogen atom represented by W include chlorine atom, fluorine atom, bromine atom and iodine atom. In particualr, chlorine atom is preferable.

Compounds (I) of the present invention include both free or pharmacologically acceptable salts thereof. When compounds (I) have an acidic group such as carboxyl group, they may form salts with inorganic bases (for example, alkali metal such as sodium and potassium, alkaline earth metal such as calcium and magnesium, transition metal such as zinc, iron and copper) or organic bases (for example, organic amines such as trimethylamine, triethylamine, pyridine, picoline, ethanolamine, diethanolamine, triethanolamine, dicyclohexylamine and N,N′-dibenzylethylenediamine, and basic amino acids such as arginine, lysine and ornithine).

When compounds (I) of the present invention have a basic group such as amino group, they may form salts with inorganic acids or organic acids (for example, hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid), or acidic amino acids such as aspartic acid and glutamic acid.

The pro-drug of compound (I) or a salt thereof means a compound which is converted to compound (I) or a salt thereof under the physiological condition or with a reaction due to an enzyme, an gastric acid, etc. in the living body, that is, a compound which is converted to compound (I) or a salt thereof with oxidation, reduction, hydrolysis, etc. according to an enzyme; a compound which is converted to compound (I) or a salt thereof with gastric acid, etc.; etc.

Examples of the pro-drug of compound (I) or a salt thereof include a compound wherein an amino group of compound (I) or a salt thereof is substituted with acyl, alkyl, phosphoric acid, etc. (e.g. a compound wherein an amino group of compound (I) or a salt thereof is substituted with eicosanoyl, alanyl, pentylaminocarbonyl, (5-methyl-2-oxo-1,3-dioxolen-4-yl)methoxycarbonyl, tetrahydrofuranyl, pyrrolidylmethyl, pivaloyloxymethyl, tert-butyl, etc.); a compound wherein an hydroxy group of compound (I) or a salt thereof is substituted with acyl, alkyl, phosphoric acid, boric acid, etc. (e.g. a compound wherein an hydroxy group of compound (I) or a salt thereof is substituted with acetyl, palmitoyl, propanoyl, pivaloyl, succinyl, fumaryl, alanyl, dimethylaminomethyl-carbonyl, etc.); a compound wherein a carboxyl group of compound (I) or a salt thereof is modified with ester, amide, etc. (e.g. a compound wherein a carboxyl group of compound (I) or a salt thereof is modified with ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester, methyl amide, etc.); etc. These pro-drug can be produced by per se known method from compound (I) or a salt thereof.

The pro-drug of compound (I) or a salt thereof may be a compound which is converted into compound (I) or a salt thereof under the physiological conditions as described in “Pharmaceutical Research and Development”, Vol. 7 (Drug Design), pages 163-198 published in 1990 by Hirokawa Publishing Co. (Tokyo, Japan).

In addition, compound (I) or a salt thereof may be hydrated or non-hydrated.

In addition, compound (I) or a salt thereof may be labeled with isotope (e.g. ³H, ¹⁴C, ³⁵S, ¹²⁵I, etc.), etc.

A compound represented by the formula (I) or a salt thereof has asymmetric carbons at a 3-position and a 5-position, may be a mixture or stereoisomers, or isomers may be separated by the known means. A trans isomer in which substituents at a 3-position and a 5-position are oriented in a reverse direction relative to a 7 membered ring plane is preferable and, in particular, an isomer in which absolute configuration at a 3-position is R configuration and a absolute configuration at a 5-position is S configuration is preferable. In addition, it may be racemic or optically active. An optically active isomer may be separated from a racemic isomer by the known optical resolution means.

Preferable examples of compound (I) of the present invention or a salt thereof are as follows:

(3R,5S)-N-propanesulfonyl-7-chloro-5-(2,3-dimethoxyphenyl)-1-(3-hydroxy-2,2-dimethylpropyl)-1,2,3,5-tetrahydro-4,1-benzoxazepine-3-acetamide, or a salt thereof

(2R)-2-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminopropionic acid, or a salt thereof,

3-[3-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminophenyl]propionic acid, or a salt thereof, or

4-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminobutanoic acid, or a salt thereof,

trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl-1-cyclohexanecarboxylic acid, or a salt thereof,

trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylphenyl]propionic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid, or a salt thereof,

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]propionic acid, or a salt thereof,

2-[2-[[[(3R,5S)-7-chloro-5-(2,3-dimethoxyphenyl)-1-(3-hydroxypropyl-2,2-dimethylpropyl)-2-oxo-1,2,3,5-tetrahydro-4,1-benzoxazepin-3-yl]acetyl]amino]ethyl]furan-3-carboxylic acid, or a salt thereof,

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid, or a salt thereof, or

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid, or a salt thereof

4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxylphenyl]butanoic acid, or a salt thereof,

5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid, or a salt thereof,

5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid, or a salt thereof, and the like.

Although a compound represented by the aforementioned formula (I) or a salt thereof can be prepared, for example, by the methods disclosed in EPA567026, WO95/21834 (PCT application based on Japanese Patent Application No. H6(1994)-15531), EPA645377 (application based on Japanese Patent Application No. H6(1994)-229159), EPA645378 (application based on Japanese Patent Application No. H6(1994)-229160) or an equivalent method, it can be prepared, for example, by the following method.

That is, a compound of the formula (I) or a salt thereof can be prepared by reacting a corresponding 3-positional carboxymethyl compound (II), or a salt thereof or a reactive derivative of a carboxyl group thereof, with a compound represented by the formula:

H₂N—R¹

wherein each symbol is as defined above, or a salt thereof, for example, as shown by the following formula. Examples of the reactive derivative of a carboxyl group include active ester, acid anhydride and acid halide (such as acid chloride).

As a salt of a compound (II), the same salts as the aforementioned salts of a compound (I) are used.

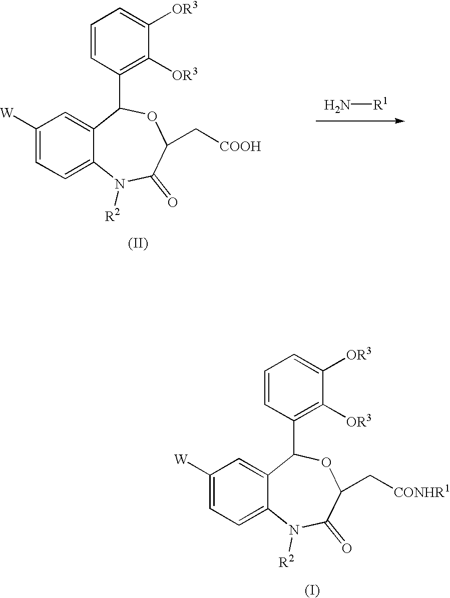

or a salt thereof or a salt

thereof

wherein each symbol is as defined above.

The reaction can be advantageously performed, for example, in a solvent, preferably in the presence of a base by using a condensing agent. Examples of the solvent used include hydrocarbon solvents such as benzene, toluene, hexane and heptane, halogenated solvents such as dichloromethane, dichloroethane, chloroform and carbon tetrachloride, ether solvents such as ethyl ether, tetrahydrofuran and dioxane, and acetonitrile and dimethylformamide. As the base, organic amines such as triethylamine, 4-dimethylaminopyridine, triethylenediamine, tetramethylethylenediamine, and 1,8-diazabicyclo[5,4,0]unde-7-cene are used. Examples of the condensing agent include condensing agents used in peptide synthesis, for example, dicyclohexylcarbodiimide, diethyl cyanophosphonate and 1-ethyl-3-(3-dimethylaminopropyl)carbodiimide, etc.

A compound represented by the formula:

H₂N—R¹

wherein R¹ is as defined above, or a salt thereof, is used usually at an amount of about 0.5 to about 2 mole equivalent, preferably about 1.0 to about 1.2 mole equivalent and, when a base is used, usually at an amount of about 0.7 to about 5 mole equivalent, preferably about 1.0 to about 2.5 mole equivalent and, when a condensing agent is used, usually at an amount of about 0.5 to about 5 mole equivalent, preferably about 1.0 to 2 mole equivalent, relative to about 1 mole of a compound represented by the formula (II), or a salt thereof or a reactive derivative thereof. A reaction temperature is usually about 0 to 100° C., preferably about 20 to 50° C., and a reaction time is usually about 0.5 to 24 hours, preferably about 1 to 5 hours.

A racemic modification of a compound used in the aforementioned reaction or a salt thereof can be obtained, for examples by a method described in WO95/21834 or an equivalent method thereto. An optically active form of a compound (II) or a salt thereof can be obtained by the optical resolution means known per se, for example, by reacting the aforementioned racemic modification with an optically active amino acid ester or a derivative thereof to produce an amido linkage and, thereafter, separating and purifying the optically active isomer using distillation, recrystallization, column chromatography or the like and, thereafter, cutting again the amido linkage.

Alternatively, enzymatic asymmetric hydrolysis is performed by a step represented by the formula:

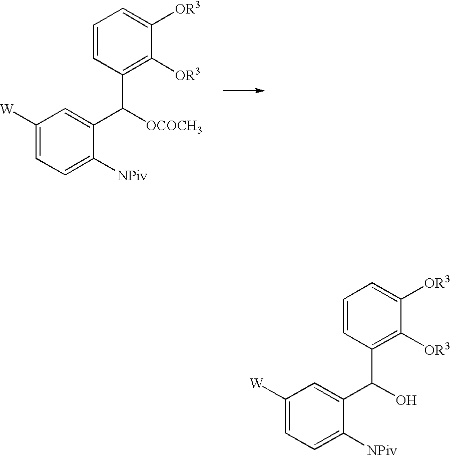

wherein Piv is pivaloyl group, and other symbols are as defined above, to obtain an optically active isomer (S form) of a benzyl alcohol derivative and, by using this optically active isomer as a starting isomer, a (3R,5S) form of the aforementioned compound (II) or a salt thereof according to a method described in EPA567026.

Alternatively, asymmetric reduction is performed by a step represented by the formula:

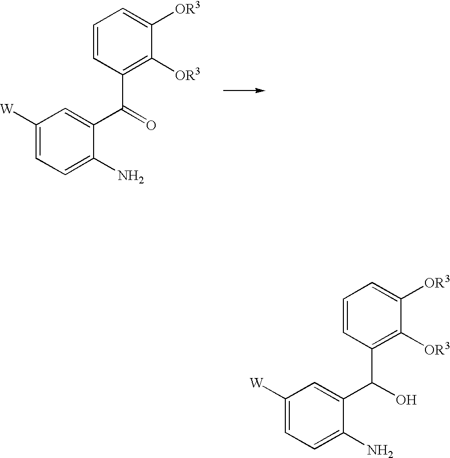

wherein each symbol is as defined above, to obtain an optically active isomer (S form) of a benzyl alcohol derivative and, by using this optically active isomer as a starting isomer, a (3R,5S) form of the aforementioned compound (II) or a salt thereof according to a method described in EPA567026.

Alternatively, when raw material compounds have an amino group, a carboxyl group or a hydroxyl group as a substituent in each reaction of a process for producing the aforementioned compounds (I) and (II) or salts thereof or in each reaction for synthesizing raw material compounds, a protecting group which is generally used in peptide chemistry may be introduced into these groups and, after the reaction, a protecting group can be removed, as necessary, to obtain an end compound.

As a protecting group for an amino group, for example, formyl, optionally substituted C₁₋₆ alkylcarbonyl (for example, acetyl and ethylcarbonyl), phenyl carbonyl, C₁₋₆ alkyl-oxycarbonyl (for example, methoxycarbonyl and ethoxycarbonyl), phenyloxycarbonyl, C₇₋₁₀ aralkyl-carbonyl (for example, benzylcarbonyl), trityl, phthaloyl and N,N-dimethylaminomethylene are used. As a substituent for them, a halogen atom (for example, fluorine, chlorine, bromine and iodine), C₁₋₆ alkyl-carbonyl (for example, methylcarbonyl, ethylcarbonyl and butylcarbonyl) and nitro group are used, and the number of substituents is around 1 to 3.

As a protecting group for carboxyl group, for example, optionally substituted C₁₋₆ alkyl (for example, methyl, ethyl, n-propyl, i-propyl, n-butyl and tert-butyl), phenyl, trityl and silyl are used. As a substituent for them, a halogen atom (for example, fluorine, chlorine, bromine and iodine), C₁₋₆ alkyl-carbonyl (for example, acetyl, ethylcarbonyl and butylcarbonyl) and nitro group are used, and the number of substituents is around 1 to 3.

As a protecting group for a hydroxy group, for example, optionally substituted C₁₋₆ alkyl (for example, methyl, ethyl, n-propyl, i-propyl, n-butyl and tert-butyl), phenyl, C₇₋₁₀ aralkyl (for example, benzyl), formyl, C₁₋₆ alkyl-carbonyl (for example, acetyl and ethylcarbonyl), phenyloxycarbonyl, benzoyl, C₇₋₁₀ aralkyl-carbonyl (for example, benzylcarbonyl), pyranyl, furanyl and silyl are used. As a substituent for them, a halogen atom (for example, fluorine, chlorine, bromine and iodine), C₁₋₆ alkyl (for example, methyl, ethyl and n-propyl), phenyl, C₇₋₁₀ aralkyl (for example, benzyl) and nitro group are used, and the number of substituents is around 1 to 4.

In addition, as a method for removing a protecting group, the method known per se or an equivalent method is used. For example, a method by treating with an acid, a base, reduction, the ultraviolet light, hydrazine, phenylhydrazine, sodium N-methyldithiocarbamate, tetrabutylammonium fluoride or palladium acetate is used.

Compounds (I) and (II) or salts thereof obtained by the above methods can be isolated and purified by the conventional separating means such as recrystallization, distillation, chromatography. When the thus obtained compound (I) of the present invention is a free compound, it can be converted into a salt by the method known per se or an equivalent method thereto (for example, neutralization). Conversely, when the compound (I) is obtained as a salt, it can be converted into a free compound or other salt by the method known per se or an equivalent method thereto. When the resulting compound is a racemic modification, it can be separated into a d-form and a 1-form.

Since a compound represented by the formula (I) or a salt thereof, and a prodrug thereof in the present invention (hereinafter, the compound (I) including a salt thereof and a prodrug thereof are simply referred to as a compound of the formula (I) or a compound (I) in some cases) are low toxic, have the squalene synthase inhibiting activity and the triglyceride lowering activity, and have the excellent lipid lowering activity, they are useful as a safe drug for preventing and/or treating hyperlipidemia such as hypercholesterolemia and hypertriglycerolemia in mammals (e.g., mouse, rat, rabbit, dog, cat, cattle, pig, monkey, human being, etc.), and are useful as a safe drug for preventing and/or treating renal diseases such as nephritis and nephropathy, atherosclerosis, arteriosclerosis, ischemic diseases, myocardial infarction, angina, aneurysm, cerebral arteriosclerosis, peripheral arteriosclerosis, thrombosis, hypertension, osteoporosis, diabetes mellitus (for example, type based on insulin resistance), pancreatic disorders, and restenosis after percutaneous transluminal coronary angioplasty (PTCA).

The utility of the present invention is explained in detail as follows.

A compound of the formula (I) has the excellent triglyceride lowering activity and the cholesterol lowering activity as well as their biological properties, and therefore, it is suitable for treating or preventing hyperlipidemia, in particular, hypertriglyceridemia, hyperlipoproteinemia and hypercholesterolemia as well as atherosclerotic blood lesion derived therefrom and their secondary diseases, for example, coronary arterial diseases, cerebral ischemia, intermittent claudication and gangrene.

In treatment of these diseases, compounds of the formula (I) may be used alone for treatment, or may be used in combination with the other drug ingredient such as the other lipid lowering drug or a cholesterol lowering drug (by simultaneous administration or administration at different times) and, in this case, these compounds are preferably administered as an oral preparation, or alternatively, may be administered in the form of suppository as a rectal preparation, if necessary. Examples of ingredients which can be combined include PPARα agonists such as fibrates [for example, clofibrate, bezafibrate, gemfibrozil, fenofibrate, Wy-1463, GW9578 and the like], nicotinic acid, and derivatives and analogues thereof [for example, acipimox and the like] and probucol and derivatives and analogues thereof [for example, CGP2881 and the like], bile acid binding resin [for example, cholestyramine, cholestypol and the like], compounds which inhibit cholesterol absorption [for example, sitosterol and neomycin and the like], compounds which inhibit cholesterol biosynthesis [for example, HMG-COA reductase inhibiting drugs such as lovastatin, simastatin, pravastatin, atorvastatin, ZD-4522, itavastatin and the like], and squalene epoxidase inhibiting drugs [for example, NB-598 and analogous compounds].

Further, other ingredients which can be combined include oxidosqualene-lanosterolcyclase, for example, decalin derivatives, azadecalin derivatives and indane derivatives.

In addition, compounds of the formula (I) are suitable for treating diseases associated with hyperchylomicronemia, for example, acute pancreatitis. Regarding the mechanism of development of pancreatitis, it is said that minute thrombus occurs in pancreatic blood capillary by chylomicron, or that free fatty acids which are produced by decomposition of triglyceride by pancreatic lipase are increased due to hyperchylomicronemia and strongly stimulate topical irritation. Therefore, since compounds of formula (I) of the present invention have the triglyceride lowering activity, they can treat pancreatitis and, thus, they can be used for treating pancreatitis alone or in combination with the known treating method. For treating present diseases, the present compounds (I) or salts or prodrugs thereof can be administered orally or topically, or they can be used alone or in combination with the known active compounds. Examples of ingredients which can be combined in this case include aprotinin (trasylol), gabexate mesylate (FOY), nafamostat mesylate (futhan), citicoline (nicholine), urinastatin (miraclide) and the like for anti-enzyme treatment. In addition, for the purpose of removing pain, anticholinergic drugs, non-narcotic analgesics, and narcotic drugs are used

An example of application of compounds of the formula (I) which is notable is secondary hyperlipidemia. This includes diabetes mellitus, hyperthyroidism, nephrotic syndrome and chronic renal failure. Hyperlipidemia is developed by these diseases and, in many cases, hyperlipidemia forms so-called vicious circle which exacerbates these diseases. Taking the lipid lowering activity into consideration, compounds of the formula (I) are suitable for treating these diseases and preventing aggravation of these diseases. Upon this, they can be administered alone or in combination with following drugs.

They can be used preferably by oral administration by combining with:

Diabetes mellitus treating drugs: kinedak, avandia benfil, humulin, euglucon, glimicron, daonil, novorin, monotard, insulins, glucobay, dimelin, rastinon, bacilcon, deamiline S, iszilins;

Hyperthyroidism treating drugs: dried thyroid (thyreoid), levothyroxine sodium (thyradin S), liothyronine sodium (cylonine, cylomin);

Nephrotic syndrome treating drugs: prednisolone (predonine), prednisolone sodium succinate (predonine), methylprednisolone sodium succinate (solu-medrol) betamethasone (rinderon);

Anti-coagulant therapy agent: dipyridamole (bersantine), dilazep hydrochloride (comelian) and the like;

Chronic renal failure treating drugs: diuretic [for example, furosemide (lasix), bumetamide (lunetoron), azosemide (diart)], hypotensive drug (for example, ACE inhibiting drug, (enalapril maleate (renivace)) and Ca antagonist (maninhilone), α receptor blocking drug.

Since hyperlipidemia exacerbates arterial sclerosis and causes hypertension, compounds of the formula (I) are also suitable for treating and/or preventing hypertension. Upon this, compounds of the formula (I) can be administered alone or in combination with the following drugs. Examples of a possible combination in this case include angiotensin-II antagonist [for example, losartan potassium (nu-lotan), candesartan cilexetil (blopress) and the like], ACE inhibiting drug [for example, enalapril maleate (renivace), lisinopril (zestril, longes), delapril (adecut), captopril and the like], calcium antagonist [for example, amlodipine tosylate (amlodin, norvasc), manidipine hydrochloride (calslot) and the like], hypotensive diuretic, α receptor blocking drug, β receptor blocking drug and the like.

Further notable indication is osteoporosis accompanied with increase in blood cholesterol. Compounds of the formula (I) can be used for treating and/or preventing osteoporosis accompanied with increase in blood cholesterol due to their excellent lipid lowering activity. Upon this, compounds of the formula (I) can be administered alone or in combination with the following drugs. Examples of a possible combination in this case include sex hormones and associated drugs [for example, estrogen preparations, ipriflavone (osten), raloxifene, osatelone, tibolone and the like], calcitonins, vitamin D preparations [for example, alpha calcidol, calcitriol and the like], bone resorption inhibitor such as bisphosphonates (for example, etidronate, chlodronate and the like), and osteogenesis promoting agent such as fluorine compound, PTH and the like.

The aforementioned known compounds which inhibit a squalene synthase, compounds which inhibit squalene synthases which are respectively described in WO9504025 WO0000458, WO98029380, WO9812170, JP-A H10(1998)-298134, JP-A H10(1998)-298177, JP-A H10(1998)-316634, Bioorganic & Medicinal Chemistry Letters, Vol.39, 2971-2979 (1996) and The Journal of Pharmacology and Experimental Therapeutics, Vol.281, 746-752(1997) can be also used for preventing and/or treating osteoporosis like the present compounds of the formula (I).

A further possible use of the present compounds of the formula (I) is inhibition of thrombus formation. The blood triglyceride level and factor VII involved in blood coagulation are positively correlated and uptake of ω-3 fatty acids lower triglyceride and at the same time inhibit coagulation and, thus, hypertriglycemia promotes thrombus formation. In addition, since VLDL of a hyperlipidemic patient more strongly increased secretion of plasminogen activator inhibitor from vascular endothelial cells than VLDL of a normolipidemia subject, it is also considered that triglyceride (hereinafter, TG) lowers the fibrinolytic ability. Therefore, in view of the TG lowering activity, compounds of the formula (I) are suitable for preventing and/or treating thrombus formation. Upon this, they can be used preferably by oral administration, alone or in combination with the following known treating drugs.

Thrombus formation preventing drugs: blood coagulation inhibitors [for example, heparin sodium, heparin calcium, warfarin calcium (warfarin)], thrombolytic drugs [for example, urokinase], anti-platelet drugs [for example, aspirin, sulfinpyrazolo (anturane), dipyridamole (persantine), acropidin (panaldin), cilostazol (pletaal)].

Further, compound (I) of the present invention has an excellent high-density lipoprotein-cholesterol increasing activity and is low toxic. Therefore, these compounds and salt thereof can be safely used as, for example, in addition to agents for preventing and/or treating primary hypo-high-density lipoprotein-cholesterolemia, Tangier disease, etc., agents for preventing and/or treating myocardial infarction, atheroscleotic diseases, arteriosclerotic diseases, hyperlipidemia, diabetes mellitus, complications of diabetes mellitus and the like in mammals (e.g., mouse, rat, hamster, rabbit, cat, dog, cattle, horse, sheep, monkey, human being, etc.). Then, they can be used for treating and/or preventing atherosclerosis, arteriosclerosis, hyperlipidemia, diabetes mellitus, its complications, diabetic nephropathy, diabetic neuropathy, diabetic retinopathy, arrhythmia, peripheral blood vessel diseases, thrombosis, pancreatic disorder, ischemic heart diseases, cerebral ischemia, post-myocardial infarction syndrome, valvular disease, Alzheimer's disease and the like. In addition, they are suitable for treating and preventing ischemic heart diseases a lot of which occur in patients with primary hypo-high-density lipoprotein-cholesterolemia, Tangier disease and postmenopausal diabetes mellitus. Further, they are suitable for treating and preventing hyperlipidemia, in particular, hypertriglyceridemia, hyperlipoproteinemia, and hypercholesterolemia, as well as atherosclerotic lesions caused therefrom and their secondary diseases, for example, coronary arterial disease, cerebral ischemia, aneurysm, cerebral arterioslerosis, peripheral arteriosclerosis, intermittent claudication, gangrene and the like.

An example of further application of the compounds represented by the formula (I) of the present invention which is notable is prevention and/or treatment of Alzheimer's disease. Increase in blood cholesterol is known to be a risk factor of Alzheimer's disease. The compounds represented by the formula (I), salts and prodrugs thereof can be used for preventing and/or treating Alzheimer's disease due to their excellent high-density lipoprotein-cholesterol increasing and lipid lowering activities. For this purpose, they can be administered alone or in combination with the following exemplified drugs. Possible combination is those with, for example, acetylcholine esterase inhibitor (e.g., ARICEPT, EXELON, etc.), an agent for inhibiting production and/or secretion of amyloid β protein (e.g., γ or β secretase inhibitor such as JT-52, LY-374973, etc., or SIB-1848, etc.), amyloid β aggregation inhibitor (e.g., PTI-00703, BETABLOC (AN-1792), etc.) and the like.

Further, since the compounds represented by the formula (I) of the present invention exhibit a blood glucose lowering activity and show a blood glucose lowering activity in obese type diabetes rats, they improve insulin resistance. Taking their biological properties into consideration, they are particularly suitable for treating and/or preventing hyperglycemia and secondary diseases caused therefrom, for example, complications observed in diabetic nephropathy and renal insufficiency, anemia, abnormal bone metabolism, vomiting, vomiturition, inappetence, diarrhea, etc., neurosis such as neuropathy, diabetic neuropathy, diabetic retinopathy, diabetic angiopathy as well as insulin resistance and diseases caused therefrom, for example, hypertension, and abnormal glucose tolerance, and further their secondary diseases, for example, malum cordis, cerebral ischemia, intermittent claudication, necropathy, etc.

The agent for increasing high-density lipoprotein-cholesterol of the present invention can be used alone or in combination with other blood glucose lowering agents or hypotensors as an agent for treating and/or preventing these diseases. In this case, preferably, these compounds are administered in the form of preparations for oral administration and, if necessary, they can be administered in the form of preparations for rectal administration or a suppository. Examples of possible components to be combined with include (1) insulin preparation (e.g., human insulin, etc.), (2) sulfonyl urea preparation (e.g., glibenclamide, gliclazide, etc.), (3) α-glucosidase inhibitor (e.g., voglibose, acarbose, etc.), (4) insulin sensitivity enhancer (e.g., pioglitazone, troglitazone, etc.), (5) aldose reductase inhibitor (e.g., epalrestat, tolurestat, etc.), (6) glycation inhibitor (e.g., aminoguanidine, etc.), and the like.

It is also possible to be combined with an agent for gyniatrics (an agent for treating menopausal diseases (binding estrogen, estradiol, testosterone enanthate/estradiol valerate, etc.), an agent for treating breast cancer (tamoxifen citrate, etc.), an agent for treating emdometriosis and/or hysteromyoma (leuproreline acetate, danazol, etc.) and the like, or combination of these drugs with an agent for treating diabetes.

Further, it is possible to be combined with a hypotensor. Examples thereof include (1) a diuretic (e.g., furosemide, supironolactone, etc.), (2) a sympathetic nerve inhibitor (e.g., atenolol, etc.), (3) an angiotensin II antagonist (e.g., losartan, candesartan cilexetil, etc.), (4) an angiotensin I converting enzyme inhibitor (e.g., enalapril maleate, delapril hydrochloride, etc.), (5) an calcium antagonist (e.g., nifedipine, manidipine hydrochloride, etc.), and the like.

The compounds of the formula (I) can be used orally or non-orally by injection, drip, inhalation or rectal administration, or topical administration. They can be used as they are, or as preparations for pharmaceutical compositions (for example, powders, granules, tablets, pills, capsules, injections, syrups, emulsions, elixirs, suspensions, solutions). That is, at least one present compound can be used alone or by mixing with a pharmaceutically acceptable carrier (adjuvant, excipient, supplementary agent and/or diluent).

Compositions for medicines can be formulated into preparations according to the conventional method. Such the preparations can be usually prepared by mixing/kneading an active ingredient with additives such as excipients, diluents, carriers and the like. Non-oral administration as used herein includes subcutaneous injection, intravenous injection, intramuscular injection, intraperitoneal injection and a drip infusion. Injectable compositions, for example, aqueous suspensions or oily suspensions for aseptic injection can be prepared using suitable dispersing agents or wetting agents or suspending agents according to the methods known in the art. The sterile injectable composition may be a solution or a suspension injectable under sterile conditions in a non-toxic diluent or solvent which can be non-orally administered such as aqueous solutions. Examples of acceptable vehicles or solvents which can be used include water, Ringer's solution, isotonic saline solution and the like. Further, a sterile non-volatile oil can also be employed as a common solvent or a suspending solvent. For this purpose, any non-volatile oils or fatty acids may be used. Natural, synthetic or semi-synthetic fatty oils or fatty acids, and natural or synthetic or semi-synthetic mono- or di- or triglycerides may be included.

Suppositories for rectal administration can be prepared by mixing the drug with suitable non-irritable excipients which are solid at a normal temperature and a liquid at an intestine tract temperature, and melt in rectum and release a drug, such as cocoa butter and polyethylene glycols.

As a solid dosage preparation for oral administration, there are aforementioned powders, granules, tablets, pills, and capsules. Such the preparations can be prepared by mixing and/or kneading an active ingredient compounds with at least one additive, for example, sucrose, lactose, cellulose sugar, mannitol (D-mannitol), multitol, dextrin, starches, (for example, corn starch), microcrystalline cellulose, agar, alginates, chitins, chitosans, pectins, tragacanth gums, acacia, gelatins, collagens, casein, albumin, synthetic or semi-synthetic polymers or glycerides. Such the preparations can also contain further additives as usual, such as inert diluents, lubricants such as magnesium stearate, preservatives such as parabens and sorbins, antioxidants such as ascorbic acid, α-tocopherol and cysteine, disintegrants (for example, floscaromerose sodium), binders (for example, hydroxypropyl cellulose), thickening agents, buffer, sweetener, flavor and perfuming agent. Tablets and pills may also be prepared by further enteric coating. Examples for liquids for oral administration include pharmaceutically acceptable emulsions, syrups, elixirs, suspending agents and solutions. They may contain inert diluents which are normally used in the art, for example, water and, if necessary, additives. These oral liquids can be prepared by mixing an active ingredient compound and an inert diluent and, if necessary, other additives according to the conventional method.

For oral administration, it is suitable to usually incorporate the present active ingredient compound at an amount of about 0.01 to 99 wt %, preferably about 0.1 to 90 wt %, usually about 0.5 to 50 wt %, depending upon dosage forms.

A dose for a certain patient is determined depending upon age, weight, general physical condition, sex, diet, administration time, administration method, excretion rate, combination of drugs, and degree of condition of disease which is being treated at that time of a patient, or taking other factors into consideration.

Lipid lowering agents such as triglyceride lowering agents and the like, which contain the present compound (I), are low toxic and can be used safely. A dose per day is different depending upon condition and weight of a patient, kind of a compound, route of administration and the like. A dose per day per adult (weight 60 kg) when used as an agent for preventing and/or treating hyperlipidemia is about 1 to 500 mg, preferably about 10 to 200 mg as an active ingredient [compound (I)] in the case of an oral agent, and about 0.1 to 100 mg, preferably about 1 to 50 mg, usually about 1 to 20 mg in the case of a non-oral agent. No toxicity is observed in this range.

The following Examples, Preparation Examples and Test Examples illustrate the present invention in more detail but are not to be construed to limit the scope thereof.

¹H NMR spectrum was measured by Varian Gemini 200 (200 MHz) type spectrometer using tetramethylsilane as an internal standard, and total 6 value is shown in ppm. Numerical values in a mixed solvent are a volumetric mixing ratio of respective solvents unless otherwise indicated. % means % by weight unless otherwise indicated. In addition, a ratio of eluting solvents in silica gel chromatography indicates a volumetric ratio unless otherwise indicated. A room temperature (normal temperature) as used herein denotes a temperature of about 20° C. to about 30° C.

Respective symbols in Examples denote the following meanings.

Ac: acetyl, Prⁿ: n-propyl, Me: methyl, Buⁿ: n-butyl, Et: ethyl, Pr^{l}: isopropyl, Et₂O: diethyl ether, s, singlet, d: doublet, t: triplet, q: quartet, dd: double doublet, dt: double triplet, m: multiplet, br: broad, J: coupling constant.

### EXAMPLE 1

(3R,5S)-N-propanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide

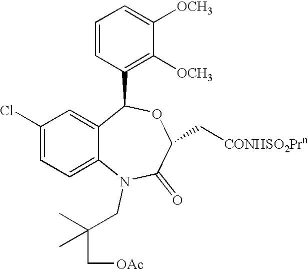

(1) A mixture of (3R,5S)-7-chloro-1,2,3,5-totrahydro-1-(3-hydroxy-2,2-dimethylpropyl)-5-(2,3-dimethoxyphenyl)-2-oxo-4,1-benzoxazepine-3-acetic acid (JP-A H09(1997)-136880, Example 11-(4), 1.1 g, 2.30 mmol), acetic anhydride (0.52 g, 5.06 mmol), 4-dimethylaminopyridine (100 mg) and pyridine (11 ml) was stirred at room temperature for 30 minutes. This mixture was diluted with ethyl acetate (100 ml), and washed with 1N hydrochloric acid, water and an aqueous saturated ammonium chloride solution. After dried with sodium sulfate, concentration under reduced pressure afforded (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.2 g, 2.31 mmol, 100%) as a colorless amorphous powder.

[α]_{D}²²−197.3° (c=0.22, methanol).

IR νₘₐₓ (KBr) cm⁻¹: 3600-2400 (br, COOH), 1736, 1678 (C═O).

¹H-NMR (CDCl₃) δ: 0.943 (3H, s), 1.022 (3H, s), 2.026 (3H, s), 2.819 (1H, dd, J=5.4, 16.4 Hz), 3.081 (1H, dd, J=7.8, 16.4 Hz), 3.553 (1H, d, J=14.0 Hz), 3.616 (3H, s), 3.732 (1H, d, J=11.4 Hz), 3.857 (1H, d, J=11.4 Hz), 3.888 (3H, s), 4.331 (1H, dd, J=5.4, 7.8 Hz), 4.578 (1H, d, J=14.0 Hz), 6.259 (1H, s), 6.647 (1H, s), 6.98-7.34 (5H, m).

(2) Thionyl chloride (0.67 g, 5.61 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) and N,N-dimethylformamide (0.03 ml) in tetrahydrofuran (10 ml) at room temperature. After stirred for 1 hour, this mixture was concentrated under reduced pressure. The residue was dissolved in tetrahydrofuran (3 ml), and this solution was added dropwise to a mixture of 1-propanesulfonamide (0.35 g, 2.81 mmol), 4-dimethylaminopyridine (0.37 g, 2.99 mmol) and tetrahydrofuran (3 ml). After stirred at room temperature for 2 hours, water was added to this mixture, and tetrahydrofuran was distilled off. The residue was dissolved in ethyl acetate (50 ml), washed with 1N hydrochloric acid and saturated brine, dried with sodium sulfate and concentrated. The residue was purified by silica gel column chromatography [eluent:ethyl acetate-hexane (2:1)] to obtain (3R,5S)-N-propanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (1.06 g, 1.70 mmol, 88%) as an amorphous powder.

[α]_{D}²²−151.9° (c=0.41, methanol).

IR νₘₐₓ (KBr) cm⁻¹: 3400-2600 (br, NH), 1732, 1678 (C═O).

¹H-NMR (CDCl₃) δ: 0.954 (3H, t, J=7.5 Hz), 0.954 (3H, s), 1.013 (3H, s), 1.76-1.96 (2H, m), 2.033 (3H, s), 2.87-2.90 (2H, m), 3.30-3.40 (2H, m), 3.556 (1H, d, J=14.4 Hz), 3.617 (3H, s), 3.709 (1H, d, J=11.4 Hz), 3.863 (1H, d, J=11.4 Hz), 3.894 (3H, s), 4.345 (1H, t, J=5.8 Hz), 4.567 (1H, d, J=14.4 Hz), 6.270 (1H, s), 6.681 (1H, d, J=2.2 Hz), 6.97-7.42 (5H, m), 9.217 (1H, br).

Elemental analysis (C₂₉H₃₇N₂O₉SCl.H₂O) Cal'd: C, 54.15; H, 6.11; N 4.36 Found: C, 53.90; H, 6.07; N, 4.67

### EXAMPLE 2

(3R,5S)-N-propanesulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide

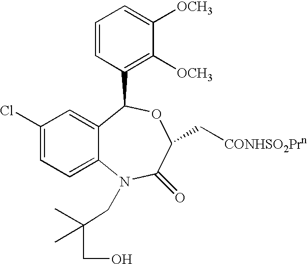

A mixture of (3R,5S)-N-propanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (0.64 g, 1.02 mmol) obtained in Example 1-(2), a 1N aqueous sodium hydroxide solution (2.5 ml) and ethanol (6 ml) was stirred at 60° C. for 30 minutes. The mixture was diluted with water (50-ml), 1N hydrochloric acid was added to adjust pH to 3 or lower (hereinafter, this procedure is referred to as “after acidification” in some cases), extracted with ethyl acetate (50 ml) 2 times. The mixture was washed with an aqueous saturated ammonium chloride solution, dried with sodium sulfate, and concentrated under reduced pressure. The residue was purified by recrystallization from ethyl acetate-hexane (1:3) to obtain (3R,5S)-N-propanesulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (0.50 g, 0.857 mmol, 84%) as a colorless powder.

mp.135-137° C.

[α]_{D}²²−171.2° (c=0.31, methanol).

IR νₘₐₓ (KBr) cm⁻¹: 3600-2500 (br, OH, NH), 1716, 1651 (C═O).

¹H-NMR (CDCl₃) δ: 0.658 (3H, s), 1.033 (3H, t, J=7.4 Hz), 1.051 (3H, s), 1.76-1.95 (2H, m), 2.77-2.98 (2H, m), 3.15-3.25 (1H, m), 3.33-3.44 (3H, m), 3.574 (1H, d, J=14.6 Hz), 3.596 (3H, s), 3.887 (3H, s), 4.389 (1H, t, J=6.0 Hz), 4.460 (1H, d, J=14.6 Hz), 6.180 (1H, s), 6.652 (1H, d, J=1.8 Hz), 6.97-7.43 (5H, m), 9.290 (1H, br).

Elemental analysis (C₂₇H₃₅N₂O₈SCl) Cal'd: C, 55.62; H, 6.05; N, 4.80. Found: C, 55.27; H, 5.82; N, 4.63.

### EXAMPLE 3

(3R,5S)-N-butanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide

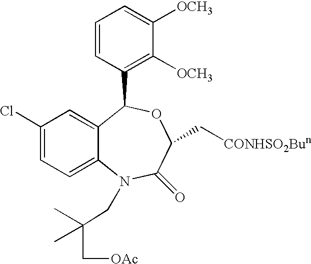

Thionyl chloride (0.67 g, 5.61 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) and N,N-dimethylformamide (0.03 ml) in tetrahydrofuran (10 ml) at room temperature. After stirred at room temperature for 1 hour, this solution was concentrated under reduced pressure, and the residue was dissolved in tetrahydrofuran (3 ml). This solution was added dropwise to a mixture of butanesulfonamide (0.39 g, 2.81 mmol), 4-dimethylaminopyridine (0.37 g, 2.99 mmol) and tetrahydrofuran (3 ml). After stirred at room temperature for 2 hours, water was added to this mixture, and tetrahydrofuran was distilled off. The residue was dissolved in ethyl acetate (50 ml), washed with 1N hydrochloric acid and saturated brine, dried with sodium sulfate and concentrated. The residue was purified by silica gel column chromatography [eluent:ethyl acetate-hexane (1:1)] to obtain (3R,5S)-N-butanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (1.06 g, 1.66 mmol, 86%) as a colorless amorphous powder.

[α]_{D}²² −130.4° (c=0.21, methanol).

IR νₘₐₓ (KBr) cm⁻¹: 3400-2500 (br, NH), 1728, 1674 (C═O).

¹H-NMR (CDCl₃) δ: 0.875 (3H, t, J=7.0 Hz), 0.954 (3H, s), 1.013 (3H, s), 1.26-1.46 (2H, m), 1.63-1.89 (2H, m), 2.031 (3H, s), 2.86-2.90 (2H, m), 3.08-3.16 (1H, m), 3.34-3.41 (1H, m), 3.554 (1H, d, J=14.4 Hz), 3.614 (3H, s), 3.707 (1H, d, J=11.4 Hz), 3.862 (1H, d, J=11.4 Hz), 3.894 (3H, s), 4.344 (1H, t, J=5.9 Hz), 4.563 (1H, d, J=14.4 Hz), 6.273 (1H, s), 6.682 (1H, d, J=2.2 Hz), 6.97-7.37 (5H, m), 9.144 (1H, br).

### EXAMPLE 4

(3R,5S)-N-butanesulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide

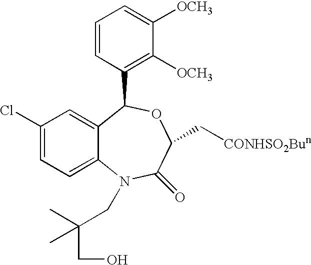

A mixture of (3R,5S)-N-butanesulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (0.8 g, 1.25 mmol), a 1N aqueous sodium hydroxide solution (2.5 ml) and ethanol (8 ml) was stirred at 60° C. for 1 hour. This mixture was diluted with water (50 ml) and, after acidification, extracted with ethyl acetate (50 ml) 2 times. This was washed with an aqueous saturated ammonium chloride solution, dried with sodium sulfate and concentrated under reduced pressure. The residue was purified by recrystallization from ethyl acetate-hexane (1:1) to obtain (3R,5S)-N-butanesulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (0.60 g, 1.00 mmol, 80%) as colorless prisms.

mp.123-125° C.

[α]_{D}²²−153.5° (c=0.20, methanol).

IR νₘₐₓ (KBr) cm⁻¹: 3600-2500 (br, OH, NH), 1716, 1653 (C═O).

¹H-NMR (CDCl₃) δ: 0.658 (3H, s), 0.925 (3H, t, J=7.0 Hz), 1.051 (3H, s), 1.38-1.50 (2H, m), 1.72-1.84 (2H, m), 2.828 (1H, dd, J=5.4, 15.4 Hz), 2.935 (1H, dd, J=6.2, 15.4 Hz), 3.13-3.24 (1H, m), 3.35-3.43 (3H, m), 3.579 (1H, d, J=15.0 Hz), 3.601 (3H, s), 3.889 (3H, s), 4.36-4.49 (2H, m), 6.186 (1H, s), 6.653 (1H, d, J=2.2 Hz), 6.97-7.42 (5H, m), 9.00-9.15 (1H, br).

Elemental analysis (C₂₈H₃₇N₂O₈SCl.0.5H₂O) Cal'd: C, 55.48; H, 6.32; N, 4.62. Found: C, 55.28; H, 6.12; N, 4.24.

### EXAMPLE 5

(3R,5S)-N-(3-acetoxypropyl)sulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide

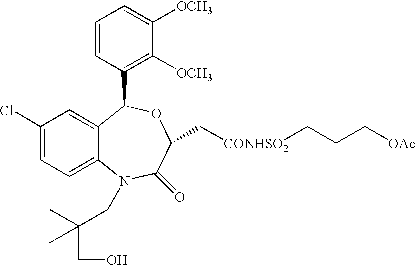

(1) A mixture of 3-acetoxypropyl bromide (13 g, 71.9 mmol), thiourea (6.0 g, 79.2 mmol) and ethanol (20 ml) was stirred at 100° C. for 1 hour. The solvent was distilled off under reduced pressure, the residue was dissolved in water (100 ml), and a chlorine gas was introduced into this aqueous solution at 0° C. for 20 minutes. The precipitate was extracted with ethyl acetate (100 ml), excess chlorine gas was distilled off, and washed with a 5% aqueous sodium hydrogen sulfite solution 3 times. After washed with saturated brine, drying with sodium sulfate and concentration afforded (3-acetoxypropyl)sulfonic acid chloride as a colorless oil. This oil was dissolved in tetrahydrofuran, (10 ml), and a concentrated aqueous ammonia (28%, 10 ml) under ice-cooling. This mixture was stirred at room temperature for 30 minutes, and extracted with ethyl acetate (50 ml). The extract was washed with saturated brine, dried with sodium sulfate, and concentrated under reduced pressure. The residue was purified by silica gel column chromatography [eluent: hexane-ethyl acetate (1:1)] to obtain 3-acetoxypropylsulfonamide (3.0 g, 16.6 mmol, 23%) as a colorless oil.

IR νₘₐₓ (KBr) cm⁻¹: 3700-3500 (br, NH), 1732 (C═O).

¹H-NMR (CDCl₃) δ: 2.079 (3H, s), 2.14-2.28 (2H, m), 3.19-3.26 (2H, m), 4.215 (2H, t, J=6.2 Hz), 4.82-5.00 (2H, br).

(2) Thionyl chloride (0.67 g, 5.61 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) and N,N-dimethylformamide (0.03 ml) in tetrahydrofuran (10 ml) at room temperature. After stirred for 1 hour, this mixture was concentrated under reduced pressure. The residue was dissolved in tetrahydrofuran (3 ml), and this solution was added dropwise to a mixture of 3-acetoxypropylsulfonamide (0.42 g, 2.30 mmol) obtained in Example 5-(1), 4-dimethylaminopyridine (0.37 g, 2.99 mmol) and tetrahydrofuran (3 ml). After stirred at room temperature for 1 hour, water was added to this mixture, and tetrahydrofuran was distilled off. The residue was dissolved in ethyl acetate (100 ml), washed with 1N hydrochloric acid and saturated brine, dried with sodium sulfate and concentrated. The residue was purified by silica gel column chromatography (eluent:ethyl acetate) to obtain (3R,5S)-N-(3-acetoxypropyl)sulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (1.1 g, 1.61 mmol, 84%) as a colorless amorphous powder.

[α]_{D}²²−124.9° (c=0.37, methanol).

IR νₘₐₓ (KBr) cm⁻¹: 3600-2600 (br, NH), 1732, 1674 (C═O).

¹H-NMR (CDCl₃) δ: 0.958 (3H, s), 1.000 (3H, s), 2.027 (6H, m), 2.87-2.90 (2H, m), 3.43-3.52 (3H, m), 3.612 (3H, s), 3.710 (1H, d, J=11.4 Hz), 3.866 (1H, d, J=11.4 Hz), 3.894 (3H, s), 4.033 (2H, t, J=5.8 Hz), 4.062 (1H, t, J=5.8 Hz), 4.14-4.23 (1H, m), 4.350 (1H, t, J=5.8 Hz), 4.559 (1H, d, J=13.8 Hz), 6.269 (1H, s), 6.683 (1H, d, J=1.8 Hz), 6.97-7.37 (5H, m).

Elemental analysis (C₃₁H₃₉N₂O₁₁ClS.1.5H₂O) Cal'd: C, 52.43; H, 5.96; N, 3.94. Found: C, 52.44; H, 5.19; N, 4.19.

### EXAMPLE 6

(3R,5S)-N-(3-hydroxypropyl)sulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide

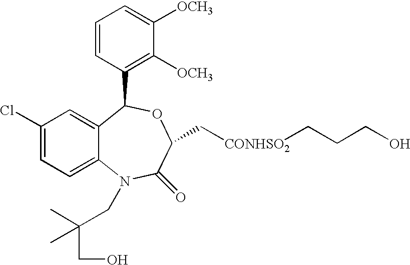

A mixture of (3R,5S)-N-(3-acetoxypropyl)sulfonyl-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (0.8 g, 1.17 mmol) obtained in Example 5, a 1N aqueous sodium hydroxide solution (4.1 ml) and ethanol (8 ml) was stirred at 60° C. for 1 hour. This mixture was diluted with water (50 ml) and, after acidification, extracted with ethyl acetate (50 ml) 2 times. This was washed with saturated brine, dried with sodium sulfate, and concentrated under reduced pressure. The residue was recrystallized from ethyl acetate-hexane (1:1) to obtain (3R,5S)-N-(3-hydroxypropyl)sulfonyl-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (0.59 g, 0.985 mmol, 84%) as a colorless powder.

mp.133-135° C.

[α]_{D}²²−177.2° (c=0.19, methanol).

IR νₘₐₓ (KBr) cm⁻¹: 3600-2400 (br, NH, OH), 1651 (C═O).

¹H-NMR (CDCl₃) δ: 0.659 (3H, s), 1.049 (3H, s), 1.99-2.13 (2H, m), 2.777 (1H, dd, J=5.2, 15.8 Hz), 2.970 (1H, dd, J=6.6, 15.8 Hz), 3.198 (1H, d, J=11.0 Hz), 3.38-3.55 (4H, m), 3.603 (3H, s), 3.714 (2H, t, J=6.2 Hz), 3.890 (3H, s), 4.36-4.47 (2H, m), 6.191 (1H, s), 6.661 (1H, s), 6.98-7.44 (5H, m), 9.4-9.6 (1H, br).

Elemental analysis (C₂₇H₃₅N₂O₉ClS.0.5H₂O) Cal'd: C, 53.33; H, 5.97; N, 4.61. Found: C, 53.31; H, 5.67; N, 4.47

### EXAMPLE 7

(3R,5S)-N-(3-phenylthiopropanesulfonyl)-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide

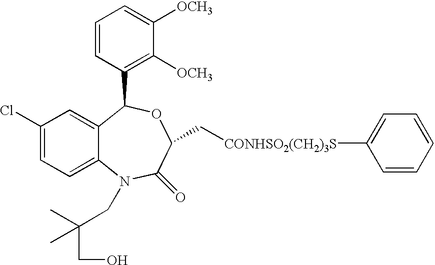

(1) A mixture of thiophenol (0.83 g, 7.53 mmol) and a 28% solution of sodium methoxide in methanol (1.59 g) and methanol (15 ml) was stirred at 60° C. for 30 minutes. A solution of 3-chloropropanesulfonamide (2.0 g, 13.1 mmol) in methanol (15 ml) was added to the above mixture, and stirred at 60° C. for 2 hours. After the solvent was distilled off under the reduced pressure, the residue was dissolved in ethyl acetate, washed with saturated brine, dried with sodium sulfate, and concentrated under reduced pressure. The residue was purified by recrystallization from ethyl acetate-hexane (1:1) to obtain 3-phenylthiopropanesulfonamide (2.6 g, 11.2 mmol, 86%) as a colorless powder.

mp.98-101° C.

IR νₘₐₓ (KBr) cm⁻¹: 3323, 3233 (NH), 1311, 1296, 1136, 896, 740, 690.

¹H-NMR (CDCl₃) δ: 2.09-2.24 (2H, m), 3.067 (2H, t, J=6.8 Hz), 3.25-3.33 (2H, m), 4.65-4.85 (2H, br), 7.22-7.40 (5H, m).

(2) Thionyl chloride (1.4 g, 11.8 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (2 g, 3.85 mmol) obtained in Example 1-(1) and N,N-dimethylformamide (0.1 ml) in tetrahydrofuran (20 ml) at room temperature. After stirred for 1 hour, this mixture was concentrated under reduced pressure. The residue was dissolved in tetrahydrofuran (10 ml), and this solution was added dropwise to a mixture of 3-phenylthiopropanesulfonamide (1.1 g, 4.75 mmol) obtained in Example 7-(1), 4-dimethylaminopyridine (0.75 g, 6.17 mmol) and tetrahydrofuran (20 ml). After stirred at room temperature for 1 hour, water was added to this mixture, and tetrahydrofuran was distilled off. The residue was dissolved in ethyl acetate (100 ml), washed with 1N hydrochloric acid and saturated brine, dried with sodium sulfate and concentrated. A 1N aqueous sodium hydroxide solution (10 ml) and ethanol (20 ml) were added to the residue, and the mixture was stirred at 60° C. for 1 hour. This mixture was diluted with water (50 ml) and, after acidification, extracted with ethyl acetate (50 ml) 2 times. This was washed with saturated brine, dried with sodium sulfate, and concentrated under reduced pressure to obtain (3R,5S)-N-(3-phenylthiopropanesulfonyl)-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (1.99 g, 2.88 mmol, 75%) as a colorless amorphous powder.

[α]_{D}²²−138.6° (c=0.26, methanol).

IR νₘₐₓ (KBr) cm⁻¹: 3600-2400 (br, NH, OH), 1714, 1651 (C═O).

¹H-NMR (CDCl₃) δ: 0.650 (3H, s), 1.042 (3H, s), 2.07-2.18 (2H, m), 2.805 (1H, dd, J=6.0, 15.4 Hz), 2.915 (1H, dd, J=7.2, 15.4 Hz), 2.999 (2H, t, J=7.0 Hz), 3.185 (1H, d, J=13.0 Hz), 3.400 (1H, d, J=14.6 Hz), 3.52-3.66 (3H, m), 3.585 (3H, s), 3.878 (3H, s), 4.380 (1H, dd, J=6.0, 7.2 Hz), 4.467 (1H, d, J=14.6 Hz), 6.175 (1H, s), 6.650 (1H, d, J=2.2 Hz), 6.96-7.43 (10H, m), 9.30-9.50 (1H, br).

Elemental analysis (C₃₃H₃₉ClN₂O₈S₂.H₂O) Cal'd: C, 55.88; H, 5.83; N, 3.95. Found: C, 56.01; H, 5.19; N, 3.96.

### EXAMPLE 8

(3R,5S)-N-(3-phenylthiopropanesulfonyl)-1(3-acetoxy-2,2-dimethylpropyl)-7-chloro-5-(2,3-dimethoxyphenyl)-2-oxo-1,2,3,5-tetrahydro-4,1-benzoxazepine-3-acetamide

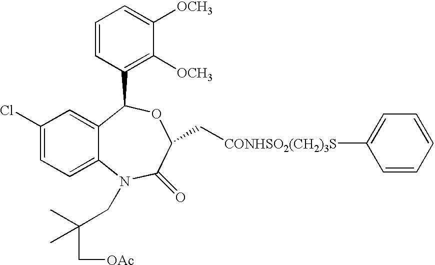

Acetyl chloride (0.40 g, 5.06 mmol) was added to a mixture of (3R,5S)-N-(3-phenylthiopropanesulfonyl)-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (1 g, 1.45 mmol) obtained in Example 7, pyridine (0.51 g, 6.50 mmol) and ethyl acetate (10 ml). After stirred at room temperature for 1 hour, water (8 ml) was added to this mixture. This mixture was stirred at room temperature for 3 hours, and extracted with ethyl acetate (50 ml) 2 times. The whole organic layer was washed with 1N hydrochloric acid (1 ml) and saturated brine (2 times), dried with sodium sulfate, concentrated under reduced pressure. The residue was purified by silica gel column chromatography [eluent:hexane-ethyl acetate (1:1)] to obtain (3R,5S)-N-(3-phenylthiopropanesulfonyl)-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (0.69 g, 0.941 mmol 65%) as a colorless amorphous powder.

[α]_{D}²²−126.3° (c=0.20, methanol).

IR νₘₐₓ (KBr) cm⁻¹: 3400-2500 (br, NH), 1732, 1658 (C═O).

¹H-NMR (CDCl₃) δ: 0.945 (3H, s), 1.003 (3H, s), 2.022 (3H, s), 2.05-2.15 (2H, m), 2.84-2.89 (2H, m), 2.928 (2H, t, J=7.0 Hz), 3.52-3.59 (3H, m), 3.614 (3H, s), 3.704 (1H, d, J=11.0 Hz), 3.860 (1H, d, J=11.0 Hz), 3.883 (3H, s), 4.329 (1H, t, J=5.2 Hz), 4.567 (1H, d, J=14.0 Hz), 6.275 (1H, s), 6.687 (1H, d, J=2.0 Hz), 6.97-7.41 (10H, m), 9.13-9.17 (1H, br).

Elemental analysis (C₃₅H₄₁ClN₂O₉S₂.0.5H₂O) Cal'd: C, 56.63; H, 5.70; N, 3.77. Found: C, 56.49; H, 5.66; N, 4.05.

### EXAMPLE 9

(3R,5S)-N-[3-(pyridin-2-yl)thiopropanesulfonyl]-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide

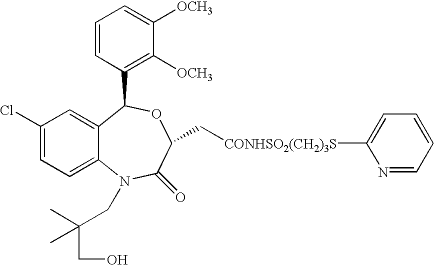

(1) A mixture of 2-mercaptopyridine (0.83 g, 7.53 mmol), a 28% solution of sodium methoxide in methanol (1.59 g) as well as methanol (15 ml) was stirred at 60° C. for 30 minutes. A solution of 3-chloropropanesulfonamide (2.0 g, 13.1 mmol) in methanol (15 ml) was added to the above mixture, and stirred at 60° C. for 2 hours. After the solvent was distilled off, the residue was dissolved in ethyl acetate, and washed with saturated brine, dried with sodium sulfate, and concentrated under reduced pressure. The residue was purified by recrystallization from ethyl acetate-hexane (1:1) to obtain 3-(pyridin-2-yl)thiopropanesulfonamide (1.4 g, 6.03 mmol, 46%) as a yellow oil.

IR νₘₐₓ (KBr) cm⁻¹: 3267 (NH), 1327, 1149, 910, 760.

¹H-NMR (CDCl₃) δ: 2.23-2.38 (2H, m), 3.302 (2H, t, J=7.2 Hz), 3.346 (2H, t, J=7.4 Hz), 5.156 (2H, br), 6.99-7.04 (1H, m), 7.193 (1H, d, J=8.2 Hz), 7.46-7.54 (1H, m), 8.407 (1H, d, J=4.4 Hz).

(2) Thionyl chloride (1.4 g, 11.8 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (2 g, 3.38 mmol) obtained in Example 1-(1) and N,N-dimethylformamide (0.1 ml) in tetrahydrofuran (20 ml) at room temperature. After stirred for 1 hour, this mixture was concentrated under reduced pressure. The residue was dissolved in tetrahydrofuran (10 ml), and this solution was added dropwise to a mixture of 3-(pyridin-2-yl)thiopropanesulfonamide (1.4 g, 6.03 mmol) obtained in Example 9-(1), 4-dimethylaminopyridine (0.75 g, 6.17 mmol) and tetrahydrofuran (20 ml). After stirred at room temperature for 1 hour, water was added to this mixture, and tetrahydrofuran was distilled off. The residue was dissolved in ethyl acetate (100 ml), washed with 1N hydrochloric acid and saturated brine, dried with sodium sulfate, and concentrated. A 1N aqueous sodium hydroxide solution (10 ml) and ethanol (20 ml) were added to the residue, and the mixture was stirred at 60° C. for 1 hour. This mixture was diluted with water (50 ml) and, after acidification, extracted with ethyl acetate (50 ml) 2 times. This was washed with saturated brine, dried with sodium sulfate, and concentrated under reduced pressure to obtain (3R,5S)-N-[3-(pyridin-2-yl)thiopropanesulfonyl]-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (1.99 g, 2.87 mmol, 75%) as a colorless amorphous powder.

[α]_{D}²²−124.7° (c=0.41, methanol).

IR νₘₐₓ (KBr) cm⁻¹: 3600-2400 (br, NH, OH), 1714, 1653 (C═O).

¹H-NMR (CDCl₃) δ: 0.652 (3H, s), 1.040 (3H, s), 2.20-2.33 (2H, m), 2.814 (1H, dd, J=6.0, 15.4 Hz), 2.943 (1H, dd, J=7.0, 15.4 Hz), 3.15-3.43 (4H, m), 3.53-3.66 (3H, m), 3.596 (3H, s), 3.881 (3H, s), 4.387 (1H, dd, J=6.0, 7.0 Hz), 4.466 (1H, d, J=14.6 Hz), 5.01-5.10 (1H, br), 6.173 (1H, s), 6.651 (1H, s), 6.96-7.47 (8H, m), 8.398 (1H, d, J=3.4 Hz), 9.16-9.66 (1H, br).

Elemental analysis (C₃₂H₃₈ClN₃O₈S₂.2H₂O) Cal'd: C, 52.78; H, 5.81; N, 5.77. Found: C, 52.77; H, 5.72; N, 6.14.

### EXAMPLE 10

(3R,5S)-N-[3-(pyridin-2-yl)thiopropanesulfonyl]-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide

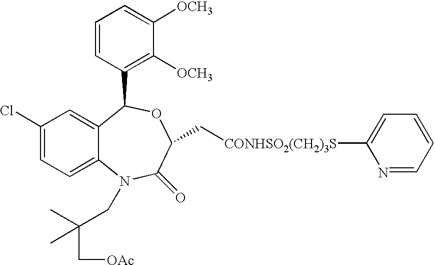

Acetyl chloride (0.40 g, 5.06 mmol) was added to a mixture of (3R,5S)-N-[3-(pyridin-2-yl)thiopropanesulfonyl]-7-chloro-5-(2,3-dimethoxyphenyl)-1-(3-hydroxy-2,2-dimethylpropyl)-2-oxo-1,2,3,5-tetrahydro-4,1-benzoxazepine-3-acetamide (1 g, 1.44 mmol) obtained in Example 9, pyridine (0.51 g, 6.50 mmol) and ethyl acetate (10 ml). After stirred at room temperature for 1 hour, water (8 ml) was added to this mixture. This mixture was stirred at room temperature for 3 hours, and extracted with ethyl acetate (50 ml) 2 times. The whole organic layer was washed with 1N hydrochloric acid (1 ml) and saturated brine 2 times, dried with sodium sulfate, and concentrated under reduced pressure. The residue was purified by silica gel column chromatography [eluent:hexane-ethyl acetate (2:1)] to obtain (3R,5S)-N-[3-(pyridin-2-yl)thiopropanesulfonyl]-1-(3-acetoxy-2,2-dimethylpropyl)-7-chloro-5-(2,3-dimethoxyphenyl)-2-oxo-1,2,3,5-tetrahydro-4,1-benzoxazepine-3-acetamide (0.63 g, 0.858 mmol, 60%) as a colorless amorphous powder.

[α]_{D}²²−114.4° (c=0.35, methanol).

IR νₘₐₓ (KBr) cm⁻¹: 3400-2400 (br, NH), 1732, 1674 (C═O).

¹H-NMR (CDCl₃) δ: 0.946 (3H, s), 1.007 (3H, s), 2.025 (3H, s), 2.15-2.33 (2H, m), 2.827 (1H, dd, J=5.2, 14.6 Hz), 2.932 (1H, dd, J=5.8, 14.6 Hz), 3.231 (2H, t, J=7.0 Hz), 3.26-3.38 (1H, m), 3.52-3.60 (2H, m), 3.625 (3H, s), 3.710 (1H, d, J=11.4 Hz), 3.861 (1H, d, J=11.4 Hz), 3.886 (3H, s), 4.346 (1H, dd, J=5.2, 5.8 Hz), 4.568 (1H, d, J=14.2 Hz), 6.277 (1H, s), 6.683 (1H, d, J=1.8 Hz), 6.95-7.54 (8H, m), 8.38-8.41 (1H, m), 9.10-9.30 (1H, br).

### EXAMPLE 11

N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-leucine

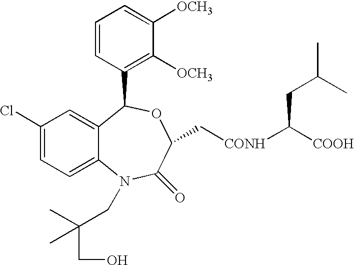

(1) Diethyl cyanophosphonate (0.41 g) and triethylamine (0.54 g) were added to a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-acetic acid (1.0 g) and L-leucine ethyl ester hydrochloride (0.49 g) in N,N-dimethylformamide (12 ml) while stirring under ice-cooling. After the reaction solution was stirred at room temperature for 30 minutes, ethyl acetate (50 ml) was added, which was washed with a 5% aqueous potassium hydrogen sulfate solution and an aqueous saturated sodium bicarbonate solution, and dried with anhydrous sodium sulfate. The solvent was concentrated under reduced pressure, and the residue was purified by silica gel column chromatography (eluent, hexane:ethyl acetate=3:2) to obtain N-[[(3R,5S)-7-chloro-5-(2,3-dimethoxyphenyl)-1-(3-hydroxy-2,2-dimethylpropyl)-1,2,3,5-tetrahydro-4,1-benzoxazepin-3-yl]acetyl]-L-leucine ethyl ester as colorless crystals.

mp.148-149° C.

¹H-NMR (CDCl₃) δ: 0.638 (3H, s), 0.917 (3H, d, J=3.6 Hz), 0.946 (3H, d, J=3.6 Hz), 1.046 (3H, s), 1.260 (3H, t, J=7.2 Hz), 1.35-1.85 (3H, m), 2.693 (1H, dd, J=5.6, 14.6 Hz), 2.913 (1H, dd, J=7.4, 14.6 Hz), 3.140 (1H, d, J=12.2 Hz), 3.369 (1H, d, J=14.2 Hz), 3.607 (3H, s), 3.608 (1H, d, J=14.2 Hz), 3.890 (3H, s), 4.160 (2H, q, J=7.2 Hz), 4.33-4.58 (3H, m), 6.13-6.22 (2H, m), 6.614 (1H, d, J=2.0 Hz), 6.95-7.39 (5H, m).

(2) A 1N sodium hydroxide (5 ml) was added to a solution of N-[[(3R,5S)-7-chloro-5-(2,3-dimethoxyphenyl)-1-(3-hydroxy-2,2-dimethylpropyl)-1,2,3,5-tetrahydro-4,1-benzoxazepin-3-yl]acetyl]-L-leucine ethyl ester (1.15 g) obtained in Example 11-(1) in tetrahydrofuran (10 ml) and methanol (20 ml), which was stirred at 60° C. for 30 minutes. The reaction solution was concentrated, neutralized with 1N hydrochloric acid, and extracted with ethyl acetate (50 ml). The organic layer was washed with an organic layer, dried with anhydrous sodium sulfate, and the solvent was concentrated under reduced pressure. From the residue, N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-leucine (0.81 g) was obtained as a colorless amorphous powder.

¹H-NMR (CDCl₃) δ: 0.640 (3H, s), 0.83-1.02 (6H, m), 1.042 (3H, s), 1.48-1.77 (3H, m), 2.715 (1H, dd, J=5.8, 14.7 Hz), 2.903 (1H, dd, J=7.2, 14.7 Hz), 3.159 (1H, d, J=12.0 Hz), 3.380 (1H, d, J=14.4 Hz), 3.597 (1H, d, J=12.0 Hz), 3.598 (3H, s), 3.882 (3H, s), 4.33-4.58 (3H, m), 6.149 (1H, s), 6.33-6.42 (1H, m), 6.618 (1H, d, J=2.0 Hz), 6.93-7.42 (3H, m).

### EXAMPLE 12

N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-leucine

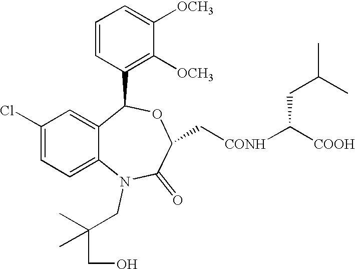

(1) Diethyl cyanophosphonate (0.61 g) and triethylamine (0.8 g) were added to a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-acetic acid (1.5 g) and D-leucine methyl ester hydrochloride (0.63 g) in N,N-dimethylformamide (15 ml) while stirring under ice-cooling. After the reaction solution was stirred at room temperature for 30 minutes, ethyl acetate (60 ml) was added, washed successively with a 5% aqueous potassium hydrogen sulfate solution, an aqueous saturated sodium bicarbonate solution and water, dried with anhydrous sodium sulfate, and concentrated. The residue was purified by recrystallization from ether to obtain N-[[(3R,5S)-7-chloro-5-(2,3-dimethoxyphenyl)-1-(3-hydroxy-2,2-dimethoxypropyl)-2-oxo-1,2,3,5-tetrahydro-4,1-benzoxazepin-3-yl]acetyl]-D-leucine methyl ester as colorless needles.

mp.110-111° C.

¹H-NMR (CDCl₃) δ: 0.643 (3H, s), 0.922 (3H, d, J=3.0 Hz), 0.949 (3H, d, J=1.6 Hz), 1.049 (3H, s), 1.42-1.85 (3H, s), 2.691 (1H, dd, J=6.0, 14.6 Hz), 2.905 (1H, dd, J=6.6, 14.6 Hz), 3.28 (1H, d, J=14.4 Hz), 3.05-3.22 (1H, m), 3.619 (3H, s), 3.722 (3H, s), 4.35-4.68 (3H, m), 6.175 (1H, s), 6.28-6.42 (1H, m), 6.608 (1H, d, J=1.6 Hz), 6.94-7.42 (5H, m).

(2) A 1N sodium hydroxide (10 ml) was added to a solution of N-[[(3R,5S)-7-chloro-5-(2,3-dimethoxyphenyl)-1-(3-hydroxy-2,2-dimethoxypropyl)-2-oxo-1,2,3,5-tetrahydro-4,1-benzoxazepin-3-yl]acetyl]-D-leucine methyl ester (1.28 g) obtained in Example 12-(1) in tetrahydrofuran (10 ml) and methanol (10 ml), which was stirred at 60° C. for 40 minutes. After the reaction solution was cooled, water (20 ml) was added, and extracted with ether (30 ml). The aqueous part was separated, a pH of the solution was adjusted with 1N hydrochloric acid to 3 or lower, extracted with ethyl acetate (40 ml), washed with water, and dried with anhydrous sodium sulfate. The solvent was concentrated under reduced pressure. From the residue, N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-leucine (1.2 g) was obtained as a colorless amorphous powder.

¹H-NMR (CDCl₃) δ: 0.646 (3H, s), 0.930 (6H, d, J=5.6 Hz), 1.033 (3H, s), 1.45-1.82 (3H, m), 2.693 (1H, dd, J=5.4, 14.5 Hz), 2.947 (1H, dd, J=7.4, 14.5 Hz), 3.178 (1H, d, J=11.8 Hz), 3.399 (1H, d, J=14.2 Hz), 3.610 (3H, s), 3.614 (1H, d, J=11.8 Hz), 4.073 (3H, s), 4.363 (1H, dd, J=5.4, 7.2 Hz), 4.451 (1H, d, J=14.2 Hz), 4.52-4.66 (1H, m), 6.158 (1H, s), 6.57-6.66 (2H, m)

### EXAMPLE 13

N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-D-leucine

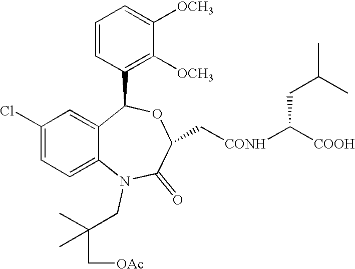

Pyridine (0.43 ml) and acetyl chloride (0.33 g) were added to a solution of N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-leucine (0.7 g) obtained in Example 12 in ethyl acetate (10 ml), which was stirred at 90° C. for at room temperature. After water (8 ml) was added to the reaction solution and stirred for 3 hours, the organic layer was separated, washed with 1N hydrochloric acid, washed with water, and dried with anhydrous sodium sulfate. The solvent was concentrated under reduced pressure, and the residue was purified by silica gel column chromatography (eluent, methylene chloride:methanol=10:1) to obtain N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-D-leucine (0.15 g) as a colorless amorphous powder.

¹H-NMR (CDCl₃) δ: 0.912 (6H, d, J=4.2 Hz), 0.952 (3H, s), 0.994 (3H, s), 1.45-1.78 (3H, m), 2.032 (3H, s), 2.699 (1H, dd, J=5.2, 14.5 Hz), 2.924 (1H, dd, J=7.2, 14.5 Hz), 3.541 (1H, d, J=14.2 Hz), 3.611 (3H, s), 3.732 (1H, d, J=11.0 Hz), 3.869 (1H, d, J=11.0 Hz), 3.894 (3H, s), 4.338 (1H, dd, J=5.4, 6.7 Hz), 4.45-4.63 (2H, m), 6.247 (1H, s), 6.63-6.72 (2H, m), 6.94-7.38 (5H, m).

### EXAMPLE 14

N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-leucine

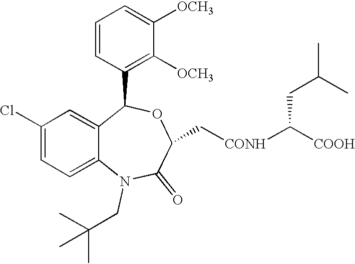

(1) Diethyl cyanophosphonate (0.42 g) and triethylamine (0.55 g) were added to a solution of (3R,5S)-7-chloro-5-(2,3-dimethoxyphenyl)-1-neopentyl-2-oxo-1,2,3,5-tetrahydro-4,1-benzoxazepine-3-acetic acid (1.0 g) and D-leucine methyl ester hydrochloride (0.47 g) in N,N-dimethylformamide (12 ml) while stirring at 0° C. After the reaction solution was stirred at room temperature for 20 minutes, water (50 ml) was added and extracted with ethyl acetate (50 ml). The organic layer was washed successively with a 5% aqueous potassium hydrogen sulfate solution, an aqueous saturated sodium bicarbonate solution and saturated brine, and dried with anhydrous sodium sulfate. The solvent was concentrated under reduced pressure and the residue was purified by silica gel chromatography (eluent, hexane:ethyl acetate=2:1) to obtain N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-leucine methyl ester as a colorless amorphous powder.

¹H-NMR (CDCl₃) δ: 0.83-1.02 (15H, m), 1.48-1.75 (3H, m), 2.695 (1H, dd, J=6.2, 14.5 Hz), 2.899 (1H, dd, J=6.6, 14.5 Hz), 3.369 (1H, d, J=13.4 Hz), 3.622 (3H, s), 3.709 (3H, s), 3.892 (3H, s), 4.362 (1H, t, J=5.8 Hz), 4.514 (1H, d, J=13.4 Hz), 4.56-4.68 (1H, m), 6.276 (1H, s), 6.35-6.46 (1H, m), 6.601 (1H, d, J=1.4 Hz), 6.95-7.38 (5H, m).

(2) A 1N sodium hydroxide (5 ml) was added to a solution of N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-leucine methyl ester (1.1 g) obtained in Example 14-(1) in tetrahydrofuran (5 ml) and methanol (10 ml), which was stirred at 60° C. for 20 minutes. Water (20 ml) was added to the reaction solution, neutralized with 1N hydrochloric acid, and extracted with ether. The organic layer was washed with water, dried with anhydrous sulfate, and concentrated under reduced pressure. From the residue, N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-leucine (0.95 g) was obtained as a colorless amorphous powder.

¹H-NMR (CDCl₃) δ: 0.83-1.02 (15H, m), 1.45-1.75 (3H, m), 2.713 (1H, dd, J=5.2, 14.2 Hz), 2.951 (1H, dd, J=7.2, 14.2 Hz), 3.370 (1H, d, J=14.0 Hz), 3.615 (3H, s), 3.891 (3H, s), 4.350 (1H, dd, J=5.2, 7.3 Hz), 4.42-4.62 (2H, m), 6.257 (1H, s), 6.608 (1H, s), 6.64-6.77 (1H, m), 6.93-7.38 (5H, m).

### EXAMPLE 15

N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-methionine

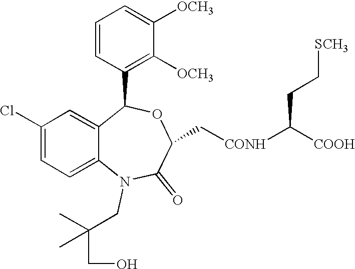

(1) Diethyl cyanophosphonate (0.41 g) and triethylamine (0.54 g) were added to a solution of (3R,5S)-7-chloro-5-(2,3-dimethoxyphenyl)-1-(3-hydroxy-2,2-dimethyl)-2-oxo-1,2,3,5-tetrahydro-4,1-benzoxazepine-3-acetic acid (1.0 g) and L-methionine methyl ester hydrochloride (0.46 g) in N,N-dimethylformamide (12 ml) while stirring at 0° C. After the reaction solution was stirred at room temperature for 30 minutes, water (30 ml) was added and extracted with ethyl acetate (50 ml). The organic layer was washed successively with a 5% aqueous potassium hydrogen sulfate solution, an aqueous saturated sodium bicarbonate solution and an aqueous sodium chloride solution, and dried with anhydrous sodium sulfate. The solvent was concentrated under reduced pressure and the precipitated crystals were filtered off by addition of ether to obtain N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-methionine methyl ester as colorless needles (0.96 g).

mp.145-146° C.

¹H-NMR (CDCl₃) δ: 0.640 (3H, s), 1.85-2.25 (2H, m), 2.102 (3H, s), 2.509 (2H, t, J=7.6 Hz), 2.710 (1H, dd, J=5.6, 14.6 Hz), 2.923 (1H, dd, J=7.8, 14.6 Hz), 3.143 (1H, d, J=12.0 Hz), 3.380 (1H, d, J=14.2 Hz), 3.579 (3H, s), 3.609 (1H, d, J=12.0 Hz), 3.736 (3H, s), 3.892 (3H, s), 4.35-4.52 (2H, m), 4.63-4.73 (1H, m), 6.155 (1H, s), 6.456 (1H, d, J=8.0 Hz), 6.617 (1H, d, J=1.8 Hz), 6.94 -7.42 (5H, m).

(2) A 1N sodium hydroxide (4 ml) was added to a solution of N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-methionine methyl ester (0.9 g) obtained in Example 15-(1) in tetrahydrofuran (5 ml) and methanol (15 ml), which was stirred at 60° C. for 40 minutes. Water (30 ml) was added to the reaction solution, and extracted with ether (30 ml). To the aqueous layer was added 1N hydrochloric acid to adjust pH of the solution to 3 or less, and extracted with ethyl acetate. The organic layer was washed with saturated brine, dried with anhydrous sodium sulfate, and concentrated under reduced pressure. The crystals obtained from the residue were recrystallized from ethyl acetate and hexane to obtain N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-L-methionine (0.76 g) as colorless prisms.

mp.129-130° C.

¹H-NMR (CDCl₃) δ: 0.644 (3H, s), 1.047 (3H, s), 1.85-2.35 (5H, m), 2.45-2.65 (2H, m), 2.729 (1H, dd, J=5.6, 14.6 Hz), 2.915 (1H, dd, J=7.4, 14.6 Hz), 3.169 (1H, d, J=12.2 Hz), 3.397 (1H, d, 15.6 Hz), 3.606 (3H, s), 3.615 (1H, d, J=12.2 Hz), 3.889 (3H, s), 4.34-4.52 (2H, m), 4.58-4.73 (1H, m), 6.159 (1H, s), 6.625 (1H, d, J=1.8 Hz), 6.64-6.73 (1H, m), 6.94-7.42 (5H, m)

### EXAMPLE 16

N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-methionine

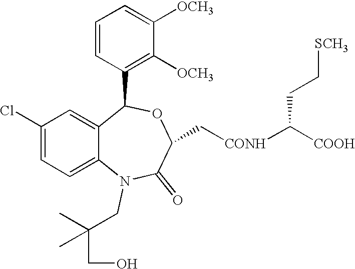

(1) (3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-acetic acid (2.0 g) and D-methionine methyl ester (1.0 g) were reaction-treated as in Example 15 to obtain N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-methionine methyl ester (1.9 g) as colorless crystals.

mp.142-143° C.

¹H-NMR (CDCl₃) δ: 0.641 (3H, s), 1.050 (3H, s), 1.85-2.25 (2H, m), 2.062 (3H, s), 2.45-2.58 (2H, m), 2.704 (1H, dd, J=6.0, 14.8 Hz), 2.925 (1H, dd, J=6.6, 14.8 Hz), 3.05-3.22 (1H, m), 3.386 (1H, d, J=14.4 Hz), 3.624 (1H, d, J=11.8 Hz), 3.625 (3H, s), 3.793 (3H, s), 4.090 (3H, s), 4.390 (1H, t, J=6.6 Hz), 4.481 (1H, d, J=14.4 Hz), 4.63-4.75 (1H, m), 6.182 (1H, s), 6.571 (1H, d, J=8.2 Hz), 6.616 (1H, d, J=1.8 Hz), 6.96-7.42 (5H, m).

(2) N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-methionine methyl ester (1.7 g) obtained in Example 16-(1) was alkali-hydrolyzed using 1N sodium hydroxide (6 ml) as in Example 15 to obtain N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-D-methionine (1.5 g) as a colorless amorphous powder.

¹H-NMR (CDCl₃) δ: 0.647 (3H, s), 1.030 (3H, s), 1.85-2.28 (2H, m), 2.051 (3H, s), 2.526 (2H, t, J=7.8 Hz), 2.706 (1H, dd, J=5.2, 14.6 Hz), 2.952 (1H, dd, J=7.4, 14.6 Hz), 3.191 (1H, d, J=11.8 Hz), 3.396 (1H, d, J=14.6 Hz), 3.613 (3H, s), 3.621 (1H, d, J=11.8 Hz), 3.892 (3H, s), 4.22-4.75 (3H, m), 6.168 (1H, s), 6.614 (1H, br), 6.85-7.43 (5H, m).

### EXAMPLE 17

N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-methionine

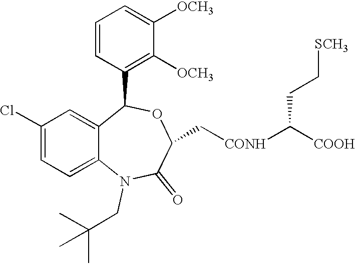

(1) (3R,5S)-7-chloro-5-(2,3-dimethoxyphenyl)-1-neopentyl-2-oxo-1,2,3,5-tetrahydro-4,1-benzoxazepine-3-aetic acid (1.5 g) and D-methionine methyl ester hydrochloride (0.71 g) were reaction-treated as in Example 15 to obtain N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-methionine methyl ester (1.6 g) as colorless crystals.

mp.103-104° C.

¹H-NMR (CDCl₃) δ: 0.946 (9H, s), 1.85-2.25 (2H, m), 2.049 (3H, s), 2.42-2.58 (2H, m), 2.709 (1H, dd, J=6.2, 14.6 Hz), 2.908 (1H, dd, J=6.6, 14.6 Hz), 3.367 (1H, d, J=14.0 Hz), 3.631 (3H, s), 2.739 (3H, s), 3.894 (3H, s), 4.377 (1H, t, J=6.4 Hz), 4.512 (1H, d, J=14.0 Hz), 4.63-4.75 (1H, m), 6.290 (1H, s), 6.58-6.68 (2H, m), 6.95-7.38 (5H, m).

(2) N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-methionine methyl ester (1.3 g) obtained in Example 17-(1) was alkali-hydrolyzed using 1N sodium hydroxide (8 ml) to obtain N-[[(3R,5S)-7-chloro-5-(2,3-dimethoxyphenyl)-1-neopentyl-2-oxo-1,2,3,5-tetrahydro-4,1-benzoxazepin-3-yl]acetyl]-D-methionine (0.92 g) as colorless crystals.

mp.161-162° C.

¹H-NMR (CDCl₃) δ: 0.938 (9H, s), 1.92-2.28 (2H, m), 2.039 (3H, s), 2.533 (2H, t, J=7.2 Hz), 2.728 (1H, dd, J=5.4, 14.5 Hz), 2.973 (1H, dd, J=7.4, 14.5 Hz), 3.372 (1H, d, J=13.8 Hz), 3.627 (3H, s), 3.895 (3H, s), 4.379 (1H, dd, J=5.4, 7.4 Hz), 4.490 (1H, d, J=13.8 Hz), 4.62-4.75 (1H, m), 6.274 (1H, s), 6.622 (1H, d, J=1.4 Hz), 6.88-7.42 (5H, m).

### EXAMPLE 18

(2R)-2-[[(3R,5S)-7-chloro-5-(2,3-dimethoxyphenyl)-1-neopentyl-2-oxo-1,2,3,5-tetrahydro-4,1-benzoxazepin-3-yl]acetyl]aminopropionic Acid

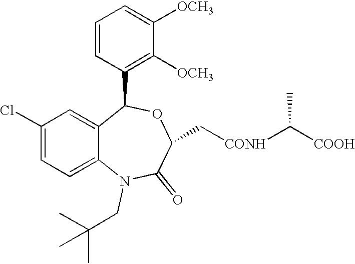

(1) Diethyl cyanophosphonate (0.39 g, 2.38 mmol) was added to a solution of (3R,5S)-7-chloro-5-(2,3-dimethoxyphenyl)-1-neopentyl-2-oxo-1,2,3,5-tetrahydro-4,1-benzoxazepine-3-acetic acid (1 g, 2.16 mmol) and D-alanine tert-butyl ester hydrochloride (0.41 g, 2.27 mmol) in N,N-dimethylformamide (10 ml) at room temperature, followed by the addition of triethylamine (0.55 g, 5.41 mmol).

This mixture was stirred at room temperature for 30 minutes, and diluted with ethyl acetate (100 ml). This was washed with a 5% potassium hydrogen sulfate, an aqueous saturated sodium bicarbonate solution and saturated brine, dried with sodium sulfate, and concentrated under reduced pressure. The residue was purified by recrystallization from ethyl acetate-hexane (1:1) to obtain tert-butyl (2R)-2-[[(3R,5S)-7-chloro-5-(2,3-dimethoxyphenyl)-1-neopentyl-2-oxo-1,2,3,5-tetrahydro-4,1-benzoxazepin-3-yl]acetyl]aminopropionate (1.3 g, 2.21 mmol, 100%) as colorless plates.

mp.127-128° C.

[α]_{D}²²−162.6° (c=0.25, methanol).

IR νₘₐₓ (KBr) cm⁻¹: 3329 (br, NH), 1732, 1678 (C═O).

¹H-NMR (CDCl₃) δ: 0.941 (9H, s), 1.376 (3H, d, J=6.8 Hz), 1.454 (9H, s), 2.679 (1H, dd, J=6.6, 14.4 Hz), 2.848 (1H, dd, J=6.2, 14.4 Hz), 3.353 (1H, d, J=14.0 Hz), 3.626 (3H, s), 3.890 (3H, s), 4.36-4.54 (3H, m), 6.287 (1H, s), 6.437 (1H, d, J=7.8 Hz), 6.594 (1H, d, J=1.4 Hz), 6.95-7.31 (5H, m).

Elemental analysis (C₃₁H₄₁N₂O₇Cl.0.5H₂O) Cal'd: C, 62.25; H, 7.08; N, 4.68 Found: C, 62.09; H, 7.08; N, 4.49

(2) (2R)-2-[[(3R,5S)-7-chloro-5-(2,3-dimethoxyphenyl)-1-neopentyl-2-oxo-1,2,3,5-tetrahydro-4,1-benzoxazepin-3-yl]acetyl]aminopropionate (0.75 g, 1.27 mmol) obtained in Example 18-(1) and trifluoroacetic acid (2 ml) were mixed, stirred at room temperature for 10 minutes, and the solvent was distilled off. The residue was purified by recrystallization to obtain (2R)-2-[[(3R,5S)-7-chloro-5-(2,3-dimethoxyphenyl)-1-neopentyl-2-oxo-1,2,3,5-tetrahydro-4,1-benzoxazepin-3-yl]acetyl]aminopropionic acid (0.53 g, 0.994 mmol, 78%) as colorless needles.

mp.184-186° C.

[α]_{D}²²−198.5° (c=0.12, methanol).

IR νₘₐₓ (KBr) cm⁻¹: 3600-2400 (br, COOH, NH), 1732, 1668 (C═O).

¹H-NMR (CDCl₃) δ: 0.939 (9H, s), 1.445 (3H, d, J=7.2 Hz), 2.710 (1H, dd, J=5.4, 14.6 Hz), 2.939 (1H, dd, J=7.4, 14.6 Hz), 3.370 (1H, d, J=13.8 Hz), 3.625 (3H, s), 3.896 (3H, s), 4.371 (1H, dd, J=5.4, 7.4 Hz), 4.493 (1H, d, J=13.8 Hz), 4.559 (1H, quintet, J=7.2 Hz), 6.277 (1H, s), 6.617 (1H, d, J=1.6 Hz), 6.703 (1H, d, J=7.2 Hz), 6.97 -7.34 (5H, m).

Elemental analysis (C₂₇H₃₃N₂O₇Cl) Cal'd: C, 60.84; H, 6.24; N, 5.26 Found: C, 60.94; H, 6.60; N, 4.99

### EXAMPLE 19

(2S)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic Acid

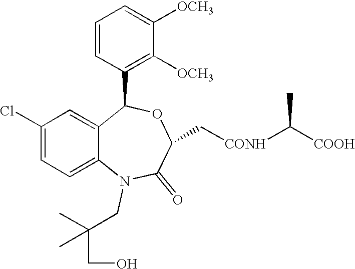

(1) Diethyl cyanophosphonate (0.19 g, 1.15 mmol) was added to a solution of (3R,5S)-7-chloro-5-(2,3-dimethoxyphenyl)-1,2,3,5-tetrahydro-1-(3-hydroxy-2,2-dimethylpropyl)-2-oxo-4,1-benzoxazepine-3-acetic acid (0.5 g, 1.05 mmol) and L-alanine ethyl ester hydrochloride (0.18 g, 1.15 mmol) in N,N-dimethylformamide (5 ml) at room temperature, followed by the addition of triethylamine (0.26 g, 2.62 mmol). This mixture was stirred at room temperature for 30 minutes, diluted with ethyl acetate (100 ml), washed with water, a 5% aqueous potassium hydrogen sulfate, an aqueous saturated sodium bicarbonate solution and saturated brine, dried with sodium sulfate, and concentrated under reduced pressure. The residue was purified by recrystallization from ethyl acetate-hexane (1:4) to obtain ethyl (2S)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionate (0.62 g, 1.07 mmol, 100%) as colorless prisms.

mp.139-132° C.

[α]_{D}²²−191.4° (c=0.17, methanol).

IR νₘₐₓ (KBr) cm⁻¹: 3600-3200 (br, OH, NH), 1739, 1655 (C═O).

¹H-NMR (CDCl₃) δ: 0.641 (3H, s), 1.044 (3H, s), 1.256 (3H, t, J=7.4 Hz), 1.396 (3H, d, J=7.4 Hz), 2.700 (1H, dd, J=5.6, 14.8 Hz), 2.903 (1H, dd, J=7.4, 14.8 Hz), 3.141 (1H, d, J=11.6 Hz), 3.374 (1H, d, J=14.6 Hz), 3.608 (3H, s), 3.610 (1H, d, J=11.6 Hz), 3.888 (3H, s), 4.183 (2H, q, J=7.4 Hz), 4.38-4.55 (3H, m), 6.159 (1H, s), 6.270 (1H, d, J=6.6 Hz), 6.610 (1H, s), 6.96-7.35 (5H, m).

Elemental analysis (C₂₉H₃₇N₂O₈Cl.H₂O) Cal'd: C, 58.53; H, 6.61; N, 4.71 Found: C, 58.27; H, 6.46; N, 4.57

(2) A mixture of ethyl (2S)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionate (0.52 g, 0.901 mmol) obtained in Example 19-(1), a 1N aqueous sodium hydroxide solution (2.5 ml) and ethanol (5 ml) was stirred at 60° C. for 30 minutes. This mixture was diluted with water (50 ml) and, after acidification, extracted with ethyl acetate (50 ml) 2 times. The whole organic layer was washed with saturated brine, dried with sodium sulfate, and concentrated under reduced pressure. The residue was purified by recrystallization from ethyl acetate-hexane (1:1) to obtain (2S)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic acid (0.44 g, 0.801 mmol, 89%) as a colorless powder.

mp.133-135° C.

[α]_{D}²²−188.5° (c=0.23, methanol).

IR νₘₐₓ (KBr) cm⁻¹: 3600-2400 (br, COOH, OH, NH), 1732, 1651 (C═O).

¹H-NMR (CDCl₃) δ: 0.645 (3H, s), 1.040 (3H, s), 1.433 (3H, t, J=7.4 Hz), 2.725 (1H, dd, J=6.2, 14.6 Hz), 2.889 (1H, dd, J=6.6, 14.6 Hz), 3.164 (1H, d, J=12.0 Hz), 3.386 (1H, d, J=14.2 Hz), 3.599 (1H, d, J=12.0 Hz), 3.601 (3H, s), 3.881 (3H, s), 4.37-4.55 (3H, m), 6.158 (1H, s), 6.475 (1H, d, J=6.6 Hz), 6.619 (1H, d, J=1.6 Hz), 6.96-7.36 (5H, m).

### EXAMPLE 20

(2S)-2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropionic Acid

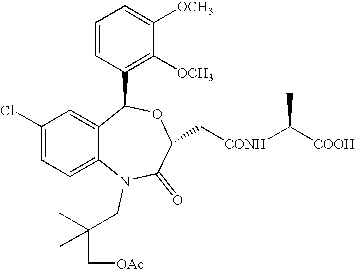

Acetyl chloride (0.12 g, 1.53 mmol) was added to a mixture of (2S)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic acid (0.24 g, 0.437 mmol) obtained in Example 19-(2), pyridine (0.16 g, 1.97 mmol) and ethyl acetate (5 ml). After stirred at 60° C. for 3 hours, water (4 ml) was added to this mixture. This mixture was stirred at room temperature overnight, and extracted with ethyl acetate (50 ml) 2 times. The whole organic layer was washed with 1N hydrochloric acid (1 ml) and saturated brine 2 times, dried with sodium sulfate, and concentrated under reduced pressured to obtain (2S)-2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropionic acid (60 mg, 0.102 mmol, 23%) as a colorless amorphous powder.

[α]_{D}²²−170.7° (c=0.13, methanol).

IR νₘₐₓ (KBr) cm⁻¹: 3600-2400 (br, COOH, NH), 1732, 1668 (C═O).

¹H-NMR (CDCl₃) δ: 0.892, 0.932, 0.987 (total 3H, each s), 1.26-1.36 (3H, m), 1.969, 2.005 (each ½×3H, s), 2.55-2.75 (1H, m), 2.80-2.95 (1H, m), 3.460 (1H, d, J=13.8 Hz), 3.575, 3.586 (total 3H, each s), 3.68-3.89 (2H, m), 3.874 (3H, s), 4.33-4.49 (3H, m), 6.227 (1H, s), 6.610 (1H, s), 6.97-7.31 (5H, m).

Elemental analysis (C₂₉H₃₅N₂O₉Cl.H₂O) Cal'd: C, 57.19; H, 6.12; N, 4.60 Found: C, 57.17; H, 5.98; N, 4.53

### EXAMPLE 21

(2R)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic Acid

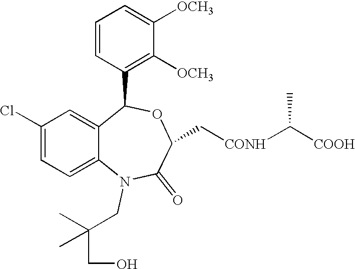

(1) Diethyl cyanophosphonate (0.19 g, 1.15 mmol) was added to a solution of (3R,5S)-7-chloro-5-(2,3-dimethoxyphenyl)-1,2,3,5-tetrahydro-1-(3-hydroxy-2,2-dimethylpropyl)-2-oxo-4,1-benzoxazepine-3-acetic acid (0.5 g, 1.05 mmol) and D-alanine methyl ester hydrochloride (0.16 g, 1.15 mmol) in N,N-dimethylformamide (5 ml) at room temperature, followed by the addition of triethylamine (0.26 g, 2.62 mmol). This mixture was stirred at room temperature for 30 minutes, diluted with ethyl acetate (100 ml), washed with water, a 5% aqueous potassium hydrogen sulfate solution, an aqueous saturated sodium bicarbonate solution and saturated brine, dried with sodium sulfate, and concentrated under reduced pressure. The residue was purified by silica gel column chromatography (eluent:ethyl acetate-hexane (2:1)A] to obtain methyl (2R)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionate (0.61 g, 1.08 mmol, 100%) as a colorless amorphous powder.

[α]_{D}²²−173.9° (c=0.27, methanol).

IR νₘₐₓ (KBr) cm⁻¹: 3600-3200 (br, OH, NH), 1743, 1660 (C═O).

¹H-NMR (CDCl₃) δ: 0.643 (3H, s), 1.048 (3H, s), 1.409 (3H, t, J=7.4 Hz), 2.679 (1H, dd, J=6.6, 14.8 Hz), 2.894 (1H, dd, J=6.8, 14.8 Hz), 3.145 (1H, d, J=10.8 Hz), 3.383 (1H, d, J=14.6 Hz), 3.57-3.66 (1H, br), 3.619 (3H, s), 3.738 (3H, s), 3.890 (3H, s), 4.381 (1H, dd, J=6.6, 6.8 Hz), 4.482 (1H, d, J=14.6 Hz), 4.564 (1H, t, J=7.4 Hz), 6.174 (1H, s), 6.428 (1H, d, J=7.8 Hz), 6.608 (1H, s), 6.96-7.35 (5H, m).

Elemental analysis (C₂₈H₃₅N₂O₈Cl.0.5H₂O) Cal'd: C, 58.79; H, 6.34; N, 4.90 Found: C, 58.67; H, 6.40; N, 4.74

(2) A mixture of methyl (2R)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionate (0.51 g, 0.906 mmol) obtained in Example 21-(1), a 1N aqueous sodium hydroxide solution (2.5 ml) and ethanol (5 ml) was stirred at 60° C. for 30 minutes. This mixture was diluted with water (50 ml) and, after acidification, extracted with ethyl acetate (50 ml) 2 times. The whole organic layer was washed with saturated brine, dried with sodium sulfate, and concentrated under reduced pressure. The residue was purified by recrystallization from ethyl acetate-hexane (1:1) to obtain (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic acid (0.37 g, 0.674 mmol, 74%) as a colorless powder.

mp.130-132° C.

[α]_{D}²²−173.9° (c=0.36, methanol).

IR νₘₐₓ (KBr) cm⁻¹: 3600-2400 (br, COOH, OH, NH), 1732, 1658 (C═O).

¹H-NMR (CDCl₃) δ: 0.650 (3H, s), 1.035 (3H, s), 1.436 (3H, t, J=7.4 Hz), 2.690 (1H, dd, J=5.8, 14.6 Hz), 2.926 (1H, dd, J=7.0, 14.6 Hz), 3.177 (1H, d, J=12.2 Hz), 3.393 (1H, d, J=14.2 Hz), 3.608 (1H, d, J=12.2 Hz), 3.610 (3H, s), 3.888 (3H, s), 4.33-4.58 (3H, m), 6.163 (1H, s), 6.608 (1H, s), 6.661 (1H, d, J=7.0 Hz), 6.96-7.35 (5H, m).

### EXAMPLE 22

(2R)-2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropionic Acid

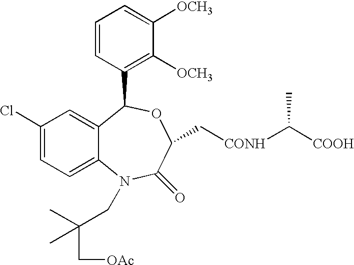

Acetyl chloride (0.10 g, 1.28 mmol) was added to a mixture of (2R)-2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropionic acid (0.20 g, 0.364 mmol) obtained in Example 21-(2), pyridine (0.13 g, 1.64 mmol) and ethyl acetate (5 ml). After stirred at 60° C. for 3 hours, water (4 ml) was added to this mixture. This mixture was stirred at room temperature overnight, and extracted with ethyl acetate (50 ml) 2 times. The whole organic layer was washed with 1N hydrochloric acid (1 ml) and saturated brine 2 times, dried with sodium sulfate, and concentrated under reduced pressure to obtain (2R)-2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropionic acid (60 mg, 0.102 mmol, 28%) as a colorless amorphous powder.

[α]_{D}²²−142.5° (c=0.11, methanol)

IR νₘₐₓ (KBr) cm⁻¹: 3600-2400 (br, COOH, NH), 1730, 1666 (C═O).

¹H-NMR (CDCl₃) δ: 0.892, 0.934, 0.989 (total 3H, each s), 1.25-1.35 (3H, m), 1.971, 2.005 (each ½×3H, s), 2.55-2.75 (1H, m), 2.85-2.95 (1H, m), 3.458 (1H, d, J=12.4 Hz), 3.577, 3.586 (total 3H, each s), 3.68-3.81 (2H, m), 3.870 (3H, s), 4.35-4.57 (3H, m), 6.227 (1H, s), 6.612 (1H, s), 6.94-7.31 (5H, m).

Elemental analysis (C₂₉H₃₅N₂O₉Cl.H₂O) Cal'd: C, 57.19; H, 6.12; N, 4.60 Found: C, 57.41; H, 5.73; N, 4.73

### EXAMPLE 23

trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid

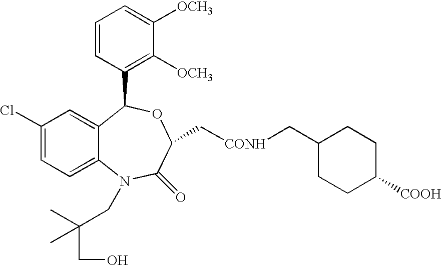

(1) Diethyl cyanophosphonate (0.37 g, 2.30 mmol) was added to a solution of (3R,5S)-7-chloro-5-(2,3-dimethoxyphenyl)-1,2,3,5-tetrahydro-1-(3-hydroxy-2,2-dimethylpropyl)-2-oxo-4,1-benzoxazepine-3-acetic acid (1 g, 2.09 mmol) and methyl tranexamate hydrochloride (0.46 g, 2.19 mmol) in N,N-dimethylformamide (10 ml) at room temperature, followed by the addition of triethylamine (0.46 g, 4.60 mmol). This mixture was stirred at room temperature for 30 minutes, and diluted with ethyl acetate (50 ml). This was washed with water, a 5% aqueous potassium hydrogen sulfate solution, an aqueous saturated sodium bicarbonate solution and saturated brine, dried with sodium sulfate, and concentrated under reduced pressure. The residue was purified by silica gel column chromatography (eluent:ethyl acetate) to obtain methyl trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylate (1.2 g, 1.98 mmol, 95%) as a colorless amorphous powder.

[α]_{D}²²−186.0° (c=0.24, methanol).

IR νₘₐₓ (KBr) cm⁻¹: 3500-3200 (br, OH), 1732, 1653 (C═O).

¹H-NMR (CDCl₃) δ: 0.639 (3H, s), 0.85-0.99 (2H, m), 1.048 (3H, s), 1.27-1.50 (3H, m), 1.77-1.84 (2H, m), 1.96-2.05 (2H, m), 2.17-2.29 (1H, m), 2.641 (1H, dd, J=6.2, 14.2 Hz), 2.837 (1H, dd, J=7.4, 14.2 Hz), 3.05-3.20 (3H, m), 3.378 (1H, d, J=14.6 Hz), 3.606 (1H, d, J=11.4 Hz), 3.608 (3H, s), 3.670 (3H, s), 3.894 (3H, s), 4.37-4.48 (2H, m), 5.912 (1H, br), 6.156 (1H, s), 6.614 (1H, d, J=2.0 Hz), 6.97-7.40 (5H, m).

Elemental analysis (C₃₃H₄₃N₂O₈Cl) Cal'd: C, 62.80; H, 6.87; N, 4.44 Found: C, 62.82; H, 7.06; N, 4.20

(2) A mixture of methyl trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylate (1.0 g, 1.58 mmol) obtained in Example 23-(1), a 1N aqueous sodium hydroxide solution (3.5 ml) and ethanol (10 ml) was stirred at 60° C. for 1 hour. This mixture was diluted with water (50 ml) and, after acidification, extracted with ethyl acetate (50 ml, 2 times), and washed with saturated brine. This was dried with sodium sulfate, and concentrated under reduced pressure. The residue was purified by silica gel column chromatography (eluent:ethyl acetate) to obtain trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid (0.50 g, 0.810 mmol, 51%) as a colorless amorphous powder.

[α]_{D}²²−194.3° (c=0.26, methanol).

IR νₘₐₓ (KBr) cm⁻¹: 3600-2400 (br, COOH, OH), 1712, 1653 (C═O).

¹H-NMR (CDCl₃) δ: 0.641 (3H, s), 0.88-1.00 (2H, m), 1.048 (3H, s), 1.26-1.53 (3H, m), 1.78-1.85 (2H, m), 2.00-2.05 (2H, m), 2.19-2.31 (1H, m), 2.648 (1H, dd, J=6.0, 14.4 Hz), 2.841 (1H, dd, J=7.0, 14.4 Hz), 3.06-3.18 (3H, m), 3.379 (1H, d, J=14.2 Hz), 3.604 (1H, d, J=11.4 Hz), 3.606 (3H, s), 3.892 (3H, s), 4.37-4.48 (2H, m), 5.958 (1H, br), 6.154 (1H, s), 6.616 (1H, d, J=2.0 Hz), 6.99-7.40 (5H, m).

Elemental analysis (C₃₂H₄₁N₂O₈Cl) Cal'd: C, 62.28; H, 6.70; N, 4.54 Found: C, 62.07; H, 6.81; N, 4.61

### EXAMPLE 24

trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl-1-cyclohexanecarboxylic acid

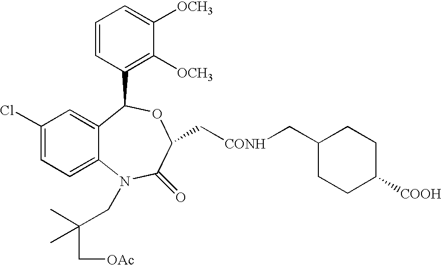

Acetyl chloride (0.13 g, 1.70 mmol) was added to a mixture of trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid (0.3 g, 0.486 mmol) obtained in Example 23-(2), pyridine (0.17 g, 2.19 mmol) and ethyl acetate (5 ml) at room temperature. After stirred at room temperature for 1.5 hours, water (5 ml) was added to this mixture. This mixture was stirred overnight, the organic layer was washed with 1N hydrochloric acid and saturated brine, dried with sodium sulfate, and concentrated under reduced pressure to obtain trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl-1-cyclohexanecarboxylic acid (0.28 g, 0.425 mmol, 87%) as a colorless amorphous powder.

[α]_{D}²²−177.9° (c=0.32, methanol).

IR νₘₐₓ (KBr) cm⁻¹: 3600-2400 (br, COOH), 1732, 1678 (C═O).

¹H-NMR (CDCl₃) δ: 0.85-1.08 (2H, m), 0.945 (3H, s), 1.013 (3H, s), 1.26-1.52 (3H, m), 1.75-1.88 (2H, m), 1.96-2.05 (2H, m), 2.029 (3H, s), 2.18-2.30 (1H, m), 2.643 (1H, dd, J=5.4, 13.8 Hz), 2.831 (1H, dd, J=7.2, 13.8 Hz), 3.05-3.15 (2H, m), 3.531 (1H, d, J=14.0 Hz), 3.608 (3H, s), 3.714 (1H, d, J=11.4 Hz), 3.861 (1H, d, J=11.4 Hz), 3.892 (3H, s), 4.376 (1H, dd, J=5.4, 7.2 Hz), 4.533 (1H, d, J=14.0 Hz), 6.061 (1H, br), 6.253 (1H, s), 6.639 (1H, d, J=2.0 Hz), 6.96-7.37 (5H, m).

Elemental analysis (C₃₄H₄₃N₂O₉Cl) Cal'd: C, 61.95; H, 6.58; N, 4.25 Found: C, 62.05; H, 6.70; N, 4.11

### EXAMPLE 25

N-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-(S)-cyclohexylalanine

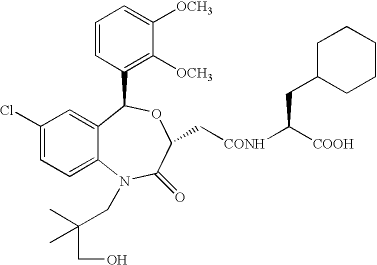

(1) (3R,5S)-7-chloro-5-(2,3-dimethoxyphenyl)-1-(3-hydroxy-2,2-dimethylpropyl-2-oxo-1,2,3,5-tetrahydro-4,1-benzoxazepine-3-acetic acid (1.0 g) and (S)-cyclohexylalanine methyl ester hydrochloride (0.51 g) were reaction-treated according to a method described in Example 15 to obtain N-[[(3R,5S)-7-chloro-5-(2,3-dimethoxyphenyl)-1-(3-hydroxy-2,2-dimethylpropyl-2-oxo-1,2,3,5-tetrahydro-4,1-benzoxazepin-3-yl]acetyl-(S)-cyclohexylalanine methyl ester (1.3 g) as a colorless amorphous powder.

¹H-NMR (CDCl₃) δ: 0.640 (3H, s), 1.046 (3H, s), 0.75-1.85 (13H, m), 2.698 (1H, dd, J=5.6, 14.4 Hz), 2.85-2.97 (1H, m), 3.05-3.45 (2H, m), 3.610 (3H, s), 3.707 (3H, s), 3.894 (3H, s), 4.15-4.68 (3H, m), 6.08-6.25 (1H, m), 6.157 (1H, s), 6.622 (1H, d, J=1.8 Hz), 6.95-7.42 (5H, m).

(2) 1N sodium hydroxide (5 ml) was added to a solution of N-[[(3R,5S)-7-chloro-5-(2,3-dimethoxyphenyl)-1-(3-hydroxy-2,2-dimethylpropyl-2-oxo-1,2,3,5-tetrahydro-4,1-benzoxazepin-3-yl]acetyl-(S)-cyclohexylalanine methyl ester (1.3 g) obtained in Example 25-(1) in tetrahydrofuran (6 ml) and methanol (15 ml), which was stirred at 60° C. for 30 minutes. The reaction solution was diluted by the addition of water (50 ml), neutralized with 1N hydrochloric acid, and extracted with ethyl acetate. The organic layer was washed with water, dried with anhydrous sodium sulfate, and concentrated. From the residue, N-[[(3R,5S)-7-chloro-5-(2,3-dimethoxyphenyl)-1-(3-hydroxy-2,2-dimethylpropyl-2-oxo-1,2,3,5-tetrahydro-4,1-benzoxazepin-3-yl]acetyl]-(S)-cyclohexylalanine (1.1 g) was obtained as a colorless amorphous powder.

¹H-NMR (CDCl₃) δ: 0.649 (3H, s), 0.75-1.83 (13H, m), 1.043 (3H, s), 2.717 (1H, dd, J=5.8, 14.5 Hz), 2.904 (1H, dd, J=7.4, 14.5 Hz), 3.162 (1H, d, J=12.2 Hz), 3.383 (1H, d, J=14.4 Hz), 3.601 (3H, s), 3.608 (1H, d, J=12.2 Hz), 4.072 (3H, s), 4.35-4.63 (3H, m), 6.153 (1H, s), 6.27-6.36 (1H, m), 6.623 (1H, d, J=2.0 Hz), 6.96-7.42 (5H, m).

### EXAMPLE 26

2-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-3-carboxylic acid

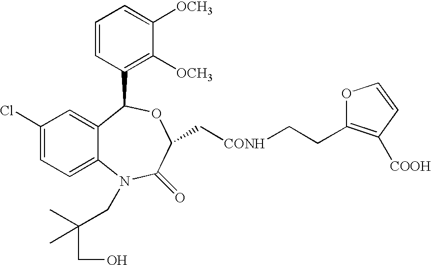

(1) Method A: 1.23 g (32.5 mmol) of sodium borohydride was added to a solution of 1.287 g (6.494 mmol) of methyl 3-methoxycarbonylfuran-2-acetate in methanol (50 ml) at room temperature, which was stirred at room temperature for 1 hour. The reaction solution was poured into water, and extracted with diethyl ether 3 times. The collected organic layers were dried with anhydrous magnesium sulfate, and the solvent was distilled off under reduced pressure. The resulting crude product was purified by silica gel column chromatography (hexane/ethyl acetate=3/1, then 1/1) to obtain methyl 2-(2-hydroxyethyl)furan-3-carboxylate.

Method B: A 1M borane-tetrahydrofuran solution (400 ml, 0.4 mol) was added dropwise to a solution of methyl 3-methoxycarbonylfuran-2-carboxylate (78.6 g, 0.4 mol) in tetrahydrofuran (150 ml) under ice-cooling, which was stirred at 70° C. for 2 hours. Water (10 ml) was added to the reaction solution to stop the reaction, and the solvent was distilled off under reduced pressure. Water (100 ml) was added to the residue, and the mixture was extracted with ethyl acetate (100 ml) 2 times. The extract was washed with 1N hydrochloric acid and an aqueous sodium bicarbonate solution, dried with anhydrous sodium sulfate, and the solvent was distilled off under residue pressure. The residue was purified by silica gel column chromatography [eluent:hexane-ethyl acetate (10:1, then 1:1)] to obtain methyl 2-(2-hydroxyethyl)furan-3-carboxylate. colorless liquid, quantum 53.3 g, yield 79%

IR νₘₐₓ (neat) cm⁻¹: 3417, 2953, 2889, 1718, 1601, 1520, 1444, 1313, 1201, 1159, 1134, 1088, 1049, 995, 744.

¹H-NMR (CDCl₃) δ: 2.21 (1H, brs), 3.27 (2H, t, J=6.2 Hz), 3.83 (3H, s), 3.93 (2H, t, J=6.1 Hz), 6.66 (1H, d, J=2.2 Hz), 7.29 (1H, d, J=2.2 Hz).

(2) Method C: A 40% solution of diethyl azodicarboxylate in toluene (100 g, 230 mmol) was added dropwise to a solution of methyl 2-(2-hydroxyethyl)furan-3-carboxylate (39.08 g, 229.7 mmol) obtained in Example 26-(1), triphenylphosphine, phthalimide (33.8 g, 230 mmol) in tetrahydrofuran (300 ml) under ice-cooling, which was stirred at room temperature overnight. The solvent of the reaction solution was distilled off under reduced pressure. The resulting residue was purified by silica gel column chromatography (hexane/ethyl acetate=1/1) to obtain methyl 2-(2-phthalimidethyl)furan-3-carboxylate. This was used in the next step without further purification.

A solution of methyl 2-(2-phthalimidethyl)furan-3-carboxylate obtained above and hydrazine monohydrate (11.1 ml, 230 mmol) in ethanol (500 ml) was heated to reflux for 1 hour. The solvent of the reaction solution was distilled off under reduced pressure, ethyl acetate was added to the residue to stir, the precipitates were filtered, and washed with ethyl acetate. The collected filtrates were concentrated, dissolved in methanol (200 ml), and treated with concentrated hydrochloric acid (25 ml). This was concentrated, ethyl acetate was added, and the produced precipitates were collected to obtain methyl 2-(2-aminoethyl)furan-3-carboxylate hydrochloride. pale brown powder, quantum 22.38 g, yield 57%

Method D: Methanesulfonyl chloride (4.88 ml, 63 mmol) was added to a solution of methyl 2-(2-hydroxyethyl)furan-3-carboxylate (10.2 g, 60 mmol) obtained in Example 26-(1) and triethylamine (11.7 ml, 84 mmol) in ethyl acetate (100 ml), which was stirred for 10 minutes. The insolubles were filtered off, and the solvent was distilled off. A mixed solution of the residue and potassium phthalimide (14.45 g, 78 mmol) and N,N-dimethylformamide (200 ml) was stirred at 110° C. for 15 hours. The reaction solution was diluted with water (1000 ml), and extracted with ethyl acetate (300 ml×3). The extract was dried with anhydrous sodium sulfate, and distilled off under reduced pressure. Hexane-ethyl acetate were added to the residue, and crystals were filtered off. The crystals were dissolved again in ethyl acetate, washed with a 2N aqueous sodium hydroxide solution, dried with anhydrous magnesium sulfate, and distilled off under reduced pressure. Hexane-diethyl ether were added to the residue, and the crystals were filtered off to obtain methyl 2-(2-phthalimidoethyl)furan-3-carboxylate (10 g, 56%).

¹H-NMR (CDCl₃) δ: 3.37 (2H, t, J=6.6 Hz), 3.68 (3H, s), 4.02 (2H, t, J=6.6 Hz), 6.62 (1H, d, J=2.0 Hz), 7.20-7.30 (1H, m), 7.65-7.78 (2H, m), 7.78-7.90 (2H, m).

A mixed solution of 2-(2-phthalimidoethyl)furan-3-carboxylate obtained above (50 g, 0.167 mmol) and hydrazine monohydrate (16.2 ml, 0.334 mmol) in ethanol (700 ml) was heated to reflux for 1 hour. The solvent was distilled off under reduced pressure, ethyl acetate (600 ml) was added, the insolubles were filtered off, and the insolubles were further washed with ethyl acetate (400 ml×3). After the ethyl acetate solutions were combined and distilled off under reduced pressure, and the residue was dissolved in methanol (20 ml). Concentrated hydrochloric acid (13.9 ml) was added under ice-cooling to produce hydrochloride, ethyl acetate-diethyl ether were added, and the precipitated crystals were filtered off to obtain methyl 2-(2-aminoethyl)furan-3-carboxylate hydrochloride (16.4 g, 48%).

¹H-NMR (CD₃OD) δ: 3.23-3.44 (4H, m), 3.84 (3H, s), 6.73 (1H, d, J=2.2 Hz), 7.52 (1H, d, J=1.8 Hz).

(3) Diethyl cyanophosphonate (4.10 ml, 27.0 mmol) was added dropwise to a solution of (3R,5S)-7-chloro-5-(2,3-dimethoxyphenyl)-1-(2,2-dimethyl-3-hydoxypropyl)-2-oxo-1,2,3,5-tetrahydro-4,1-benzoxazepine-3-acetic acid (11.7 g, 24.6 mmol), methyl 2-(2-aminoethyl)furan-3-carboxylate hydrochloride (5.05 g, 24.6 mmol) obtained in Example 26-(2), 1,8-diazabicyclo[5.4.0]undec-7-ene (4.04 ml, 27.0 mmol) and triethylamine (5.13 ml, 36.8 mmol) in tetrahydrofuran (80 ml) while stirring at room temperature, which was stirred at room temperature overnight. An aqueous sodium bicarbonate solution was added to the reaction solution, and stirred at room temperature for 1 hour. The produced precipitates were collected, washed with water, and dried to obtain methyl 2-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-3-carboxylate.

white powder, quantum 13.67 g, yield 88%

mp.81-83° C.

[α]_{D}²²−175.6° (c=0.994, CHCl₃).

¹H-NMR (CDCl₃) δ: 0.63 (3H, s), 1.04 (3H, s), 2.59 (1H, dd, J=5.5 Hz, 14.3 Hz), 2.83 (1H, dd, J=7.6, 14.2 Hz), 3.07-3.23 (3H, m), 3.35 (1H, d, J=14.2 Hz), 3.51-3.63 (3H, m), 3.60 (3H, s), 3.84 (3H, s), 3.89 (3H, s), 4.18-4.25 (1H, m), 4.35-4.45 (2H, m), 6.13 (1H, s), 6.38 (1H, brt, J=5.5 Hz), 6.59 (1H, d, J=1.8 Hz), 6.66 (1H, d, J=2.2 Hz), 6.98 (1H, dd, J=3.2, 6.6 Hz), 7.13-7.19 (2H, m), 7.27 (1H, d, J=1.8 Hz), 7.34-7.39 (2H, m); IR νₘₐₓ (KBr) cm⁻¹: 3439, 3318, 2942, 1717, 1663, 1481, 1281, 1067.

Elemental analysis (C₃₂H₃₇ClN₂O₉.1.0DMF) Cal'd: C, 59.87; H, 6.32; N, 5.98 Found: C, 59.77; H, 6.33; N, 5.76

(4) Method E: A 1N aqueous sodium hydroxide solution (40 ml) was added to a solution of methyl 2-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-3-carboxylate (12.85 g, 20.43 mmol) obtained in Example 26-(3) in methanol (100 ml), and stirred at room temperature overnight. The reaction solution was concentrated under reduced pressure, diluted with water, 1N hydrochloric acid (45 ml) was added dropwise to the resulting aqueous solution while stirring. The produced precipitates were collected, washed with water, and dried to obtain 2-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-3-carboxylic acid.

white powder, quantum 11.31 g, yield 90%

Method F: Thionyl chloride (11.7 g, 98.7 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (17 g, 32.9 mmol) obtained in Example 1-(1) and N,N-dimethylformamide (0.3 ml) in tetrahydrofuran (150 ml) at room temperature. After stirred for 1 hour, the mixture was concentrated under reduced pressure. The residue was dissolved in tetrahydrofuran (100 ml), which was added to a mixture of methyl 2-(2-aminoethyl)furan-3-carboxylate hydrochloride (8.2 g, 42.8 mmol) obtained in Example 26-(2), triethylamine (10.8 g, 107 mmol) and tetrahydrofuran (100 ml). This was stirred at room temperature for 30 minutes, and diluted with ethyl acetate (200 ml). This was washed with 1N hydrochloric acid and saturated brine, dried with sodium sulfate, and concentrated under reduced pressure. The residue was purified by silica gel column chromatography [eluent:ethyl acetate-hexane (2:1)] to obtain methyl 2-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]ethyl]furan-3-carboxylate (22.1 g, 32.9 mmol, 100%) as a colorless amorphous powder.

[α]_{D}²²−174.4° (c=0.27, methanol).

IR νₘₐₓ (KBr) cm⁻¹: 3319 (NH), 1722, 1682 (C═O).

¹H-NMR (CDCl₃) δ: 0.934 (3H, s), 1.024 (3H, s), 1.007 (3H, s), 2.024 (3H, s), 2.589 (1H, dd, J=5.8, 14.2 Hz), 2.803 (1H, dd, J=7.4, 14.2 Hz), 3.194 (2H, t, J=6.6 Hz), 3.513 (1H, d, J=14.0 Hz), 3.550 (2H, t, J=6.6 Hz), 3.597 (3H, s), 3.711 (1H, d, J=11.0 Hz), 3.823 (3H, s), 3.855 (1H, d, J=11.0 Hz), 3.887 (3H, s), 4.369 (1H, dd, J=5.8, 7.4 Hz), 4.513 (1H, d, J=14.0 Hz), 6.237 (1H, s), 3.27-6.37 (1H, br), 6.615 (1H, d, J=2.0 Hz), 6.636 (1H, t, J=1.8 Hz), 6.95-7.36 (6H, m).

Elemental analysis (C₃₄H₃₉N₂O₁₀Cl) Cal'd: C, 60.85; H, 5.86; N, 4.17 Found: C, 60.49; H, 5.79; N, 3.88

A mixture of methyl 2-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]ethyl]furan-3-carboxylate (22.1 g, 32.9 mmol) obtained above, a 1N aqueous sodium hydroxide solution (70 ml) and ethanol (140 ml) was stirred at 60° C. for 30 minutes. This was diluted with water (100 ml) and, after acidification, extracted with ethyl acetate (200 ml). This was washed with saturated brine, dried with sodium sulfate, and concentrated under reduced pressure. The residue was purified by recrystallization from ethanol-water (1:1) to obtain 2-[2-[[[(3R,5S)-7-chloro-5-(2,3-dimethoxyphenyl)-2-oxo-1-(3-hydroxy-2,2-dimethylpropyl)-1,2,3,5-tetrahydro-4,1-benzoxazepin-3-yl]acetyl]amino]ethyl]furan-3-carboxylic acid (10.4 g, 16.9 mmol) as a colorless powder.

mp.126-129° C.

[α]_{D}²²−190.2° (c=0.990, methanol).

¹H-NMR (CDCl₃) δ: 0.63 (3H, s), 1.04 (3H, s), 2.59 (1H, dd, J=5.3, 14.5 Hz), 2.85 (1H, dd, J=7.9, 14.1 Hz), 3.13-3.24 (3H, m), 3.35 (1H, d, J=14.4 Hz), 3.46-3.63 (3H, m), 3.58 (3H, s), 3.88 (3H, s), 4.34-4.45 (2H, m), 6.12 (1H, s), 6.51 (1H, t, J=5.1 Hz), 6.59 (1H, d, J=1.4 Hz), 6.69 (1H, d, J=2.0 Hz), 6.97 (1H, dd, J=3.8, 6.2 Hz), 7.13-7.17 (2H, m), 7.23-7.33 (3H, m).

IR νₘₐₓ (KBr) cm⁻¹: 3300-2500, 1659, 1481, 1283, 1063.

Elemental analysis (C₃₁H₃₅ClN₂O₉.0.5H₂O) Cal'd: C, 59.66; H, 5.81; N, 4.49 Found: C, 59.65; H, 5.87; N, 4.32

### EXAMPLE 27

2-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]ethyl]furan-3-carboxylic acid

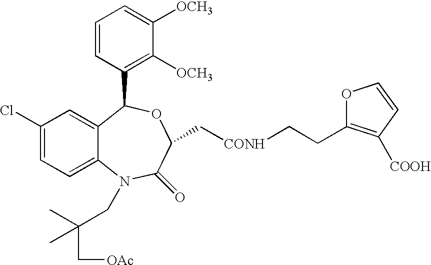

Acetyl chloride (0.31 ml, 4.34 mmol) was added dropwise to a solution of 2-[2-[[[(3R,5S)-7-chloro-5-(2,3-dimethoxyphenyl)-1(2,2-dimethyl-3-hydroxypropyl)-2-oxo-1,2,3,5-tetrahydro-4,1-benzoxazepin-3-yl]acetyl]amino]ethyl]furan-3-carboxylic acid (0.762 g, 1.239 mmol) obtained in Example 26-(4) and pyridine (0.45 ml, 5.57 mmol) in ethyl acetate (20 ml), which was stirred for 2 hours as it was. The solvent of the reaction solution was distilled off, and the resulting crude product was purified by silica gel column chromatography (hexane/ethyl acetate=1/1-ethyl acetate) to obtain 2-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]ethyl]furan-3-carboxylic acid. colorless foam, quantum 0.438 g, yield 54%

[α]_{D}²²−179.9° (c=0.993, methanol).

¹H-NMR (CDCl₃) δ: 0.93 (3H, s), 0.99 (3H, s), 2.03 (3H, s), 2.61 (1H, dd, J=5.8, 14.4 Hz), 2.83 (1H, dd, J=7.5, 14.5 Hz), 3.13-3.30 (2H, m), 3.47-3.85 (5H, m), 3.60 (3H, s), 3.88 (3H, s), 4.38 (1H, t, J=6.6 Hz), 4.51 (1H, d, J=14.4 Hz), 6.23 (1H, s), 6.46 (1H, brt, J=5.5 Hz), 6.61 (1H, s), 6.67 (1H, d, J=1.8 Hz), 6.97 (1H, t, J=4.9 Hz), 7.12-7.21 (2H, m), 7.27-7.37 (3H, m).

IR νₘₐₓ (neat) cm⁻¹: 3348, 2941, 1724, 1676, 1479, 1282, 1246, 733.

Elemental analysis (C₃₃H₃₇ClN₂O₁₀.0.5H₂O) Cal'd: C, 59.50; H, 5.75; N, 4.21 Found: C, 59.86; H, 5.89; N, 4.16

### EXAMPLE 28

5-[2-[[[(3R,5S)-7-chloro-5-(2,3-dimethoxyphenyl)-1(2,2-dimethyl-3-hydroxypropyl)-2-oxo-1,2,3,5-tetrahydro-4,1-benzoxazepin-3-yl]acetyl]amino]ethyl]furan-2,4-dicarboxylic acid

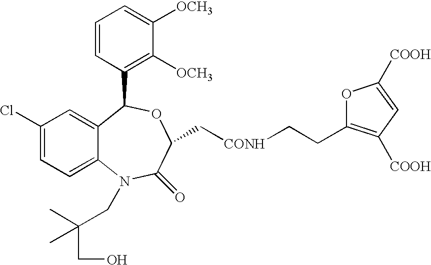

(1) Ethyl chloroformylacetate potassium salt (91.32 g, 0.4841 mol) [obtained by gradually adding t-butoxy potassium (112 g, 1 mol) to a solution of ethyl chloroformate (123 g, 1 mol) and ethyl formate (74 g, 1 mol) in diisopropyl ether (500 ml) under ice-cooling, stirring at room temperature overnight, collecting the produced precipitates, washing with diisopropyl ether, and drying it (quantum 150 g)] was added to a solution of dimethyl 3-oxoglutarate (84.30 g, 0.4841 mol) in pyridine (300 ml) at room temperature, which was stirred at 90° C. for 1 day. The reaction solution was concentrated, poured into water, and extracted with ethyl acetate 3 times. The collected organic layers were dried with anhydrous magnesium sulfate, and the solvent was distilled off under reduced pressure. The resulting crude product was purified by silica gel column chromatography (hexane/ethyl acetate=3/1-2/1) to obtain methyl 5-ethoxycarbonyl-3-methoxycarbonylfuran-2-acetate. yellow liquid, quantum 88.61 g, yield 68%

¹H-NMR (CDCl₃) δ: 1.38 (3H, t, J=7.1 Hz), 3.85 (3H, s), 4.14 (2H, s), 4.37 (2H, q, J=7.1 Hz), 7.43 (1H, s). IR νₘₐₓ (neat) cm⁻¹: 1724, 1275, 1242, 1174, 1076.

(2) A 1.0M solution of borane in tetrahydrofuran (328 ml, 0.328 mol) was added dropwise to a solution of methyl 5-ethoxycarbonyl-3-methoxycarbonylfuran-2-acetate (88.61 g, 0.3279 mol) obtained in Example 28-(1) in tetrahydrofuran (150 ml) at −78° C., which was stirred at room temperature for 8 hours. The solvent of the reaction solution was distilled off, an aqueous ammonium chloride solution was added thereto, and extracted with ethyl acetate 3 times. The collected organic layers were dried with anhydrous magnesium sulfate, and the solvent was distilled off. The resulting residue was purified by silica gel column chromatography (hexane/ethyl acetate=3/1-2/1-1/1) to obtain ethyl 5-(2-hydroxyethyl)-4-methoxycarbonylfuran-2-carboxylate. yellow liquid, quantum 36.98 g, yield 47% (raw material recovery: 24.98 g, recovery rate 28%).

¹H-NMR (CDCl₃) δ: 1.37 (3H, t, J=7.1 Hz), 2.22 (1H, brt, J=5.3 Hz), 3.34 (2H, t, J=6.2 Hz), 3.86 (3H, s), 3.99 (2H, brq, J=5.9 Hz), 4.36 (2H, q, J=7.1 Hz), 7.40 (1H, s).

IR νₘₐₓ (neat) cm⁻¹: 3440, 1720, 1263, 1236, 1174, 1076.

(3) Methanesulfonyl chloride (21.0 g, 0.183 mol) was added dropwise to a solution of ethyl 5-(2-hydroxyethyl)-4-methoxycarbonylfuran-2-carboxylate (36.98 g, 0.1527 mol) obtained in Example 28-(2) and triethylamine (31.9 ml, 0.229 mol) in diethyl ether (100 ml) under ice-cooling, which was stirred at room temperature for 0.5 hour. The produced precipitates were filtered, and washed with ethyl acetate. The solvent of the collected filtrate was distilled off under reduced pressure. The resulting residue was dissolved in N,N-dimethylformamide (300 ml), phthalimide potassium (33.9 g, 0.183 mol) was added thereto, and stirred at room temperature overnight. Water was poured into the reaction solution, and stirred at room temperature for 0.5 hour. The produced precipitates were collected by filtration, and washed with water to obtain N-[2-(5-ethoxycarbonyl-3-methoxycarbonylfuran-2-yl)ethyl]phthalimide.

white powder, quantum 44.6 g, yield 79%

mp.122-123° C.

¹H-NMR (CDCl₃) δ: 1.31 (3H, t, J=7.1 Hz), 3.43 (2H, t, J=6.4 Hz), 3.70 (3H, s), 4.07 (2H, t, J=6.4 Hz), 4.30 (2H, q, J=7.2 Hz), 7.36 (1H, s), 7.69-7.76 (2H, m), 7.79-7.85 (2H, m).

IR νₘₐₓ (KBr) cm⁻¹: 1735, 1716, 1452, 1398, 1367, 1247, 1176, 1081.

(4) A solution of N-[2-(5-ethoxycarbonyl-3-methoxycarbonylfuran-2-yl)ethyl]phthalimide (0.86 g, 2.31 mmol) obtained in Example 28-(3) and hydrazine monohydrate (0.11 ml, 2.31 mol) in ethanol (20 ml) was heated to reflux for 1 hour. The solvent of the reaction solution was distilled off, the resulting wet powder was washed with ethyl acetate, and the collected filtrates were concentrated to obtain crude ethyl 5-(2-aminoethyl)-4-methoxycarbonylfuran-2-carboxylate. Diethyl cyanophosphonate (0.38 ml, 2.52 mmol) was added dropwise to a solution of (2R,5S)-7-chloro-5-(2,3-dimethoxyphenyl)-1-(2,2-dimethyl-3-hydroxypropyl)-2-oxo-1,2,3,5-tetrahydro-4,1-benzoxazepine-3-acetic acid (1.004 g, 2.101 mmol), the crude ethyl 5-(2-aminoethyl)-4-methoxycarbonylfuran-2-carboxylate obtained above and triethylamine (0.44 ml, 3.15 mmol) in tetrahydrofuran (20 ml) while stirring at room temperature, which was stirred at room temperature overnight. Water was poured into the reaction solution, and extracted with ethyl acetate 2 times. The collected organic layers were dried with anhydrous magnesium sulfate, and the solvent was distilled off under reduced pressure. The resulting crude product was purified by silica gel column chromatography (hexane/ethyl acetate=1/1-1/3) to obtain 2-ethyl 4-methyl 5-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-2,4-dicarboxylate. colorless foam, quantum 0.832 g, yield 57%

[α]_{D}²²−153° (c=1.002, methanol).

¹H-NMR (CDCl₃) δ: 0.62 (3H, s), 1.04 (3H, s), 1.38 (3H, t, J=7.1 Hz), 1.77 (1H, brs), 2.59 (1H, dd, J=5.4, 14.6 Hz), 2.83 (1H, dd, J=8.1, 14.7 Hz), 3.13 (1H, d, J=11.6 Hz), 3.23-3.38 (3H, m), 3.52-3.65 (3H, m), 3.60 (3H, s), 3.87 (3H, s), 3.89 (3H, s), 4.29-4.41 (4H, m), 6.13 (1H, s), 6.37 (1H, brt, J=5.1 Hz), 6.59 (1H, d, J=1.4 Hz), 6.98 (1H, dd, J=2.8, 7.2 Hz), 7.12-7.19 (2H, m), 7.34-7.41 (3H, m).

IR νₘₐₓ (neat) cm⁻¹: 3375, 2954, 1718, 1655, 1479, 1279, 1234, 1171, 1070, 731.

(5) A 1N aqueous sodium hydroxide solution (4 ml) was added to a solution of 2-ethyl 4-methyl 5-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-2,4-dicarboxylate (0.616 g, 0.879 mmol) obtained in Example 28-(4) in methanol (20 ml), which was stirred at room temperature overnight. The reaction solution was concentrated under reduced pressure, diluted with water, and 1N hydrochloric acid (6 ml) was added dropwise to the resulting aqueous solution while stirring. The produced precipitates were collected, washed with water, and dried to obtain 5-[2-[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]ethyl]furan-2,4-dicarboxylic acid.

white powder, quantum 0.417 g, yield 72%

mp.155-157° C.

[α]_{D}²²−171.3° (c=1.006, methanol).

¹H-NMR (CD₃OD) δ: 0.83 (3H, s), 0.93 (3H, s), 2.65 (1H, dd, J=6.9, 14.9 Hz), 2.74 (1H, dd, J=6.6, 15.0 Hz), 3.19 (1H, dd, J=11.4 Hz), 3.26 (2H, t, J=6.6 Hz), 3.42 (1H, d, J=11.4 Hz), 3.54 (2H, t, J=6.6 Hz), 3.57 (3H, s), 3.65 (1H, d, J=14.4 Hz), 3.88 (3H, s), 4.35 (1H, t, J=6.7 Hz), 4.40 (1H, d, J=14.4 Hz), 6.15 (1H, s), 6.51 (1H, d, J=2.2 Hz), 7.07-7.25 (3H, m), 7.35 (1H, s), 7.45 (1H, dd, J=2.4, 8.8 Hz), 7.59 (1H, d, J=8.8 Hz).

IR νₘₐₓ (KBr) cm⁻¹: 3300-2500, 1715, 1655, 1481, 1283, 1173, 1065, 768.

Elemental analysis (C₃₂H₃₅ClN₂O₁₁.0.5H₂O) Cal'd: C, 57.53; H, 5.43; N, 4.19 Found: C, 57.70; H, 5.52; N, 4.07

### EXAMPLE 29

5-[[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]methyl]furan-2-carboxylic acid

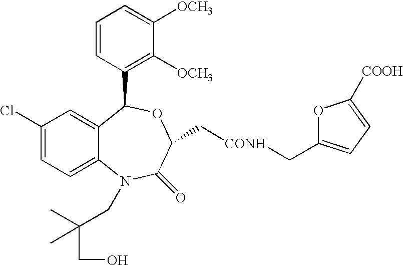

(1) Ethyl 5-(chloromethyl)furan-2-carboxylate (5.240 g, 27.78 mmol) and potassium phthalimide (5.40 g, 29.2 mmol) were stirred in N,N-dimethylformamide (30 ml) at 65° C. for 0.5 hour. Water was poured into the reaction solution, and stirred at room temperature for 0.5 hour. The produced precipitates were collected by filtration, washed with water, and dried to obtain N-[[5-(ethoxycarbonyl)furan-2-yl]methyl]phthalimide. pale brown powder, quantum 7.766 g, yield 93%

mp.108-109° C.

¹H-NMR (CDCl₃) δ: 1.35 (3H, t, J=7.2 Hz), 4.33 (2H, q, J=7.1 Hz), 4.93 (2H, s), 6.41 (1H, d, J=3.8 Hz), 7.09 (1H, d, J=3.6 Hz), 7.72-7.79 (2H, m), 7.84-7.91 (2H, m).

IR νₘₐₓ (KBr) cm⁻¹: 1715, 1406, 1393, 1296, 1148, 947, 735.

Elemental analysis (C₁₆H₁₃NO₅) Cal'd: C, 64.21; H, 4.38; N, 4.68 Found: C, 64.05; H, 4.33; N, 4.93

(2) A solution of N-[[5-(ethoxycarbonyl)furan-2-yl]methyl]phthalimide (0.70 g, 2.34 mmol) obtained in Example 29-(1) and hydrazine monohydrate (0.11 ml, 2.34 mmol) in ethanol (20 ml) was heated to reflux for 1 hour. The solvent of the reaction solution was distilled off, the resulting wet powder was washed with ethyl acetate, and the collected filtrates were concentrated to obtain ethyl 5-(aminomethyl)furan-2-carboxylate. Diethyl cyanophosphonate (0.39 ml, 2.55 mmol) was added dropwise to (3R,5S)-7-chloro-5-(2,3-dimethoxyphenyl)-1-(2,2-dimethyl-3-hydroypropyl)-2-oxo-1,2,3,5-tetrahydro-4,1-benzoxazepine-3-acetic acid (1,015 g, 2.124 mmol), the crude ethyl 5-(aminomethyl)furan-2-carboxylate obtained above and triethylamine (0.44 ml, 3.19 mmol) in tetrahydrofuran (20 ml) while stirring at room temperature, which was stirred at room temperature overnight. The reaction solution was poured into water, and extracted with ethyl acetate 2 times. The collected organic layers were dried with anhydrous magnesium sulfate, and the solvent was distilled off under reduced pressure. The resulting crude product was purified by silica gel column chromatography (eluent:hexane/ethyl acetate=1/1, then 1/3) to obtain ethyl 5-[[[[(3R,5S)-7-chloro-5-(2,3-dimethoxyphenyl)-1(2,2-dimethyl-3-hydroxypropyl)-2-oxo-1,2,3,5-tetrahydro-4-1-benzoxazepin-3-yl]acetyl]amino]methyl]furan-2-carboxylate.

white powder, quantum 1.238 g, yield 93%

Recrystallization from ethyl acetate-diethyl ether afforded white crystals.

mp.162-164° C.

[α]_{D}²²−218.1° (c=1.006, methanol).

¹H-NMR (CDCl₃) δ: 0.64 (3H, s), 1.04 (3H, s), 1.37 (3H, t, J=7.2 Hz), 2.69 (1H, dd, J=5.6, 14.4 Hz), 2.90 (1H, dd, J=7.5, 14.5 Hz), 3.14 (1H, d, J=12.0 Hz), 3.38 (1H, d, J=14.2 Hz), 3.60 (1H, d, J=11.8 Hz), 3.60 (3H, s), 3.89 (3H, s), 4.35 (2H, q, J=7.3 Hz), 4.42-4.50 (4H, m), 6.15 (1H, s), 6.35 (1H, d, J=3.2 Hz), 6.37 (1H, t, J=5.4 Hz), 6.60 (1H, d, J=1.4 Hz), 6.98 (1H, dd, J=2.2, 7.4 Hz), 7.08-7.21 (3H, m), 7.31-7.40 (2H, m).

IR νₘₐₓ (KBr) cm⁻¹: 3322, 2978-2878, 1725, 1676, 1645, 1483, 1294, 1138, 1067, 766.

Elemental analysis (C₃₂H₃₇ClN₂O₉) Cal'd: C, 61.09; H, 5.93; N, 4.45 Found: C, 61.07; H, 5.87; N, 4.38

(3) A 1N aqueous sodium hydroxide solution (2 ml) was added to a solution of ethyl 5-[[[[(3R,5S)-7-chloro-5-(2,3-dimethoxyphenyl)-1-(2,2-dimethyl-3-hydroxypropyl)-2-oxo-1,2,3,5-tetrahydro-4-1-benzoxazepin-3-yl]acetyl]amino]methyl]furan-2-carboxylate (0.754 g, 1.199 mmol) obtained in Example 29-(2) in methanol (10 ml)-tetrahydrofuran (10 ml), which was stirred at room temperature overnight. The reaction solution was concentrated under reduced pressure, diluted with water, and 1N hydrochloric acid (3 ml) was added dropwise to the resulting aqueous solution while stirring. The produced precipitates were collected, washed with water, and dried to obtain 5-[[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]methyl]furan-2-carboxylic acid.

white powder, quantum 0.471 g, yield 65%

mp.128-131° C.

[α]_{D}²²−219.3° (c=0.990, methanol)

¹H-NMR (CDCl₃) δ: 0.64 (3H, s), 1.04 (3H, s), 2.71 (1H, dd, J=5.4, 14.6 Hz), 2.92 (1H, dd, J=8.1, 14.7 Hz), 3.18 (1H, d, J=12.6 Hz), 3.38 (1H, d, J=14.4 Hz), 3.60 (3H, s), 3.60 (1H, d, J=12.4 Hz), 3.89 (3H, s), 4.39-4.49 (4H, m), 6.13 (1H, s), 6.38 (1H, d, J=3.4 Hz), 6.61 (1H, s), 6.64 (1H, t, J=5.6 Hz), 6.98 (1H, dd, J=2.4, 7.6 Hz), 7.10-7.20 (3H, m), 7.34 (2H, s).

IR νₘₐₓ (KBr) cm⁻¹: 3310, 2940, 2650-2500, 1717, 1655, 1526, 1481, 1283, 1065, 768.

Elemental analysis (C₃₀H₃₃ClN₂O₉.0.5H₂O) Cal'd: C, 59.06; H, 5.62; N, 4.59 Found: C, 59.29; H, 5.32; N, 4.59.

### EXAMPLE 30

3-[[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]methyl]furan-2-carboxylic acid

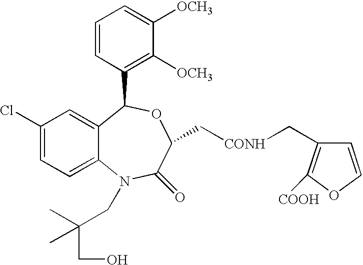

(1) A 1.6M solution of n-butyllithium in hexane (100 ml, 160 mmol) was added dropwise to a solution of furan-3-methanol (7.840 g, 79.92 mmol) in tetrahydrofuran (100 ml) at −78° C. under nitrogen stream, which was stirred for 1 hour under ice cooling. This was cooled to −78° C., 10 g of crushed dry ice was added, and a temperature was gradually raised from −78° C. to room temperature while stirring the reaction solution. The solvent of the reaction solution was distilled off, an about 10% solution of hydrogen chloride in methanol (200 ml) was added to the resulting residue, and heated to reflux overnight. The solvent of the reaction solution was distilled off under reduced pressure, and the resulting crude product was purified by silica gel column chromatography (eluent: hexane/ethyl acetate=3/1, then 1/1) to obtain methyl 3-(hydroxymethyl)furan-2-carboxylate. brown liquid, quantum 10.14 g, yield 81%

¹H-NMR (CDCl₃) δ: 3.95 (3H, s), 4.79 (2H, s), 6.55 (1H, d, J=1.8 Hz), 7.49 (1H, d, J=1.6 Hz).

IR νₘₐₓ (neat) cm⁻¹: 3411, 1713, 1443, 1308, 1200, 1014.

(2) Methanesulfonic acid chloride (5.53 ml, 71.4 mmol) was added dropwise to a solution of methyl 3-(hydroxymethyl)furan-2-carboxylate (10.14 g, 64.94 mmol) obtained in Example 30-(1) and triethylamine (13.6 ml, 97.4 mmol) in ethyl acetate (100 ml) under ice-cooling, which was stirred at room temperature for 0.5 hour. The produced precipitates were filtered, and washed with ethyl acetate. The solvent of the collected filtrates was distilled off under reduced pressure. The resulting residue was dissolved in N,N-dimethylformamide (80 ml), phthalimide potassium (33.9 g, 0.183 mol) was added, and stirred at 60° C. for 4 hours. Water was poured into the reaction solution, and stirred at room temperature for 0.5 hour. The produced precipitates were filtered to collect, washed with water, and dried to obtain N-[[2-(methoxycarbonyl)furan-3-yl]methyl]phthalimide. pale brown powder, quantum 13.18 g, yield 71%

mp.140-143° C.

¹H-NMR (CDCl₃) δ: 3.97 (3H, s), 5.15 (2H, s), 6.44 (1H, d, J=1.6 Hz), 7.45 (1H, d, J=1.8 Hz), 7.70-7.81 (2H, m), 7.84-7.91 (2H, m).

IR νₘₐₓ (KBr) cm⁻¹: 1726, 1709, 1412, 1394, 1348, 1316, 1296, 1082, 947, 814, 731, 714.

(3) A solution of N-[[2-(methoxycarbonyl)furan-3-yl]methyl]phthalimide (0.77 g, 2.68 mmol) obtained in Example 30-(2) and hydrazine monohydrate (0.13 ml, 2.68 mmol) in ethanol (20 ml) was heated to reflux for 1 hour. The solvent of the reaction solution was distilled off, the resulting wet powder was washed with ethyl acetate, and the collected filtrates were concentrated to obtain methyl 3-(aminomethyl)furan-2-carboxylate.

Diethyl cyanophosphonate (0.44 ml, 2.93 mmol) was added dropwise to (3R,5S)-7-chloro-5-(2,3-dimethoxyphenyl)-1-(2,2-dimethyl-3-hydoroxypropyl)-2-oxo-1,2,3,5-tetrahydro-4,1-benzoxazepine-3-acetic acid (1.166 g, 2.440 mmol), the crude methyl 3-(aminomethyl)furan-2-carboxylate obtained above and triethylamine (0.51 ml, 3.66 mmol) in tetrahydrofuran (20 ml) at room temperature while stirring, which was stirred at room temperature overnight. The reaction solution was poured into water, and extracted with ethyl acetate 2 times. The collected organic layers were dried with anhydrous magnesium sulfate, and the solvent was distilled off. The resulting crude product was purified by silica gel column chromatography (eluting solvent:hexane/ethyl acetate=1/1, then 1/3) to obtain crude methyl 3-[[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]methyl]furan-2-carboxylate.

A 1N aqueous sodium hydroxide solution (2 ml) was added to a solution of the compound obtained above in methanol (20 ml), and stirred at room temperature overnight. The reaction solution was concentrated under reduced pressure, diluted with water, and 1N hydrochloric acid (3 ml) was added dropwise to the resulting aqueous solution while stirring. The resulting precipitates were collected, washed with water, and dried to obtain 3-[[[[(3R,5S)-7-chloro-5-(2,3-dimethoxyphenyl)-1-(2,2-dimethyl-3-hydroxypropyl)-2-oxo-1,2,3,5-tetrahydro-4,1-benzoxazepin-3-yl]acetyl]amino]methyl]furan-2-carboxylic acid.

white powder, quantum 0.531 g, yield 36%

mp.125-128° C.

[α]_{D}²²−208.7° (c=1.004, methanol).

¹H-NMR (CDCl₃) δ: 0.63 (3H, s), 1.04 (3H, s), 2.68 (1H, dd, J=5.5, 14.3 Hz), 2.88 (1H, dd, J=7.4, 14.2 Hz), 3.19 (1H, d, J=12.2 Hz), 3.37 (1H, d, J=14.4 Hz), 3.58 (3H, s), 3.66 (1H, d, J=12.2 Hz), 3.88 (3H, s), 4.35-4.45 (2H, m), 4.53 (2H, d, J=6.2 Hz), 6.10 (1H, s), 6.55 (1H, d, J=1.8 Hz), 6.59 (1H, d, J=1.4 Hz), 6.92-6.99 (2H, m), 7.04-7.19 (2H, m), 7.33-7.39 (2H, m), 7.48 (1H, d, J=1.8 Hz).

IR νₘₐₓ (KBr) cm⁻¹: 3300-2500, 1655, 1481, 1283, 1067.

Elemental analysis (C₃₀H₃₃ClN₂O₉.0.5H₂O) Cal'd: C, 59.06; H, 5.62; N, 4.59 Found: C, 58.77; H, 5.54; N, 4.43.

### EXAMPLE 31

4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenylacetic acid

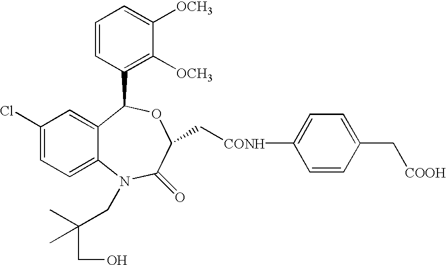

(1) Thionyl chloride (0.67 g, 5.61 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) and N,N-dimethylformamide (0.03 ml) in tetrahydrofuran (10 ml) at room temperature. After stirred for 1 hour, this mixture was concentrated under reduced pressure. The residue was dissolved in tetrahydrofuran (3 ml). This solution was added to a mixture of methyl 4-aminophenylacetate hydrochloride (0.46 g, 2.30 mmol), triethylamine (0.48 g, 4.80 mmol) and tetrahydrofuran (5 ml). This mixture was stirred at room temperature for 30 minutes. Water was added to this mixture, and extracted with ethyl acetate (50 ml). The extract was washed with saturated brine, dried with sodium sulfate, and concentrated under reduced pressure. The residue was purified by silica gel column chromatography [ethyl acetate-hexane (1:1)] to obtain methyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetate (1.21 g, 1.81 mmol, 94%) as a colorless amorphous powder.

[α]_{D}²²−130.8° (c=0.38, methanol).

IR νₘₐₓ (KBr) cm⁻¹: 1738, 1680 (C═O).

¹H-NMR (CDCl₃) δ: 0.954 (3H, s), 1.018 (3H, s), 2.024 (3H, s), 2.811 (1H, dd, J=5.4, 14.0 Hz), 2.997 (1H, dd, J=7.4, 14.0 Hz), 3.531 (1H, d, J=14.2 Hz), 3.588 (2H, s), 3.616 (3H, s), 3.683 (3H, s), 3.624 (1H, d, J=11.8 Hz), 3.873 (1H, dd, J=11.8 Hz), 3.892 (3H, s), 4.401 (1H, dd, J=5.4, 7.4 Hz), 4.553 (1H, d, J=14.2 Hz), 6.292 (1H, s), 6.639 (1H, d, J=1.8 Hz), 6.97-7.48 (9H, m), 7.880 (1H, br).

Elemental analysis (C₃₅H₃₉N₂O₉Cl) Cal'd: C, 63.01; H, 5.89; N, 4.20. Found: C, 62.66; H, 6.04; N, 4.25.

(2) A mixture of methyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetate (1.0 g, 1.50 mmol) obtained in Example 31-(1), a 1N aqueous sodium hydroxide solution (4.0 ml) and ethanol (10 ml) was stirred at 60° C. for 1 hour. This mixture was diluted with water (50 ml), and extracted with ethyl acetate (50 ml×2). The extract was washed with saturated brine, dried with sodium sulfate, and concentrated under reduced pressure. The residue was purified by recrystallization from ethyl acetate-hexane (1:1) to obtain 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenylacetic acid (0.74 g, 1,21 mmol, 81%) as colorless needles.

mp.142-144° C.

[α]_{D}²²−132.8° (c=0.25, methanol).

IR νₘₐₓ (KBr) cm⁻¹: 3600-2400 (br, COOH, OH), 1714, 1653 (C═O).

¹H-NMR (CDCl₃) δ: 0.645 (3H, s), 1.040 (3H, s), 2.820 (1H, dd, J=6.0, 14.4 Hz), 3.016 (1H, dd, J=7.2, 14.4 Hz), 3.171 (1H, d, J=11.8 Hz), 3.370 (1H, d, J=14.0 Hz), 3.607 (5H, s), 3.614 (1H, d, J=11.8 Hz), 3.889 (3H, s), 4.40-4.49 (2H, m), 6.176 (1H, s), 6.615 (1H, d, J=2.2 Hz), 6.96-7.47 (9H, m), 7.931 (1H, br).

Elemental analysis (C₃₂H₃₅N₂O₈Cl.0.5H₂O) Cal'd: C, 61.98; H, 5.85; N, 4.52 Found: C, 62.00; H, 6.25; N, 4.13

### EXAMPLE 32

4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetic acid

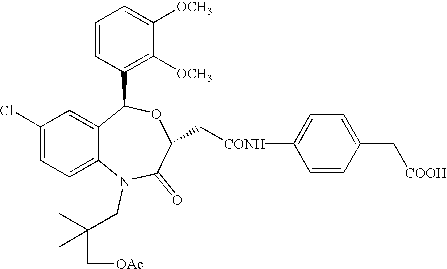

Acetyl chloride (90 mg, 1.15 mmol) was added to a mixture of 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenylacetic acid (0.2 g, 0.327 mmol) obtained in Example 31-(2), pyridine (0.12 g, 1.47 mmol) and ethyl acetate (5 ml). After stirred at room temperature for 1.5 hours, water (5 ml) was added to this mixture, and further stirred overnight. The organic layer was separated, and washed with 1N hydrochloric acid and saturated brine. This was dried with sodium sulfate, and concentrated under reduced pressure. The residue was purified by silica gel column chromatography [eluent:ethyl acetate-methanol (10:1)] to obtain 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetic acid (73 mg, 0.112 mmol, 34%) as a colorless amorphous powder.

[α]_{D}²²−136.4° (c=0.14, methanol)

IR νₘₐₓ (KBr) cm⁻¹: 3600-2400 (br, COOH, NH), 1732, 1682 (C═O).

¹H-NMR (CDCl₃) δ: 0.941 (3H, s), 1.002 (3H, s), 2.002 (3H, s), 2.813 (1H, dd, J=4.0, 14.0 Hz), 3.040 (1H, dd, J=7.8, 14.0 Hz), 3.524 (1H, d, J=13.8 Hz), 3.560 (2H, s), 3.610 (3H, s), 3.729 (1H, d, J=10.6 Hz), 3.857 (1H, d, J=10.6 Hz), 3.888 (3H, s), 4.430 (1H, dd, J=4.0, 7.8 Hz), 4.530 (1H, d, J=13.8 Hz), 6.286 (1H, s), 6.645 (1H, d, J=2.0 Hz), 6.96-7.43 (9H, m), 8.222 (1H, br).

Elemental analysis (C₃₄H₃₇N₂O₉Cl.H₂O) Cal'd: C, 60.85; H, 5.86; N, 4.17 Found: C, 61.14; H, 5.81; N, 4.35

### EXAMPLE 33

3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic Acid

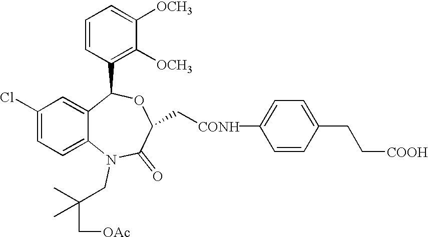

(1) Carbonyldiimidazole (12.9 g, 79.9 mmol) was added to a suspension of 4-acetylaminobenzoic acid (13 g, 72.6 mmol) in tetrahydrofuran (100 ml) at room temperature. After stirred at room temperature for 6 hours, a magnesium salt of malonic acid monoethyl ester (12.5 g, 43.6 mmol) was added to this mixture. The reaction mixture was stirred at 60° C. for 2 hours. This was diluted with ethyl acetate (100 ml), washed with an aqueous saturated ammonium chloride solution 2 times, dried with sodium sulfate, and concentrated under reduced pressure. The residue was purified by silica gel column chromatography [eluent: hexane-ethyl acetate (1:1)] to obtain ethyl 3-(4-acetylaminophenyl)-3-oxopropionate (14.77 g, 59.3 mmol, 82%) as colorless plates.

mp.93-94° C.

IR νₘₐₓ (KBr) cm⁻¹: 3483 (NH), 1743, 1714, 1674 (C═O).

¹H-NMR (CDCl₃) δ: 1.260 (9/10×3H, t, J=7.4 Hz), 1.333 (1/10×3H, t, J=7.4 Hz), 2.214 (3H, s), 3.962 (9/10×2H, s), 4.127 (1/10×2H, q, J=7.4 Hz), 4.214 (9/10, ×2H, q, J=7.4 Hz), 5.617 (1/10×1H, s), 7.632 (2H, d, J=8.8 Hz), 7.740 (1/10×2H, d, J=8.8 Hz), 7.78-7.84 (1H, br), 7.905 (9/10×2H, d, J=8.8 Hz).

Elemental analysis (C₁₃H₁₅NO₄.0.3H₂O) Cal'd: C, 61.31; H, 6.17; N, 5.50 Found: C, 61.49; H, 6.10; N, 5.55.

(2) Sodium borohydride (2.9 g, 77.1 mmol) was added to a solution of ethyl 3-(4-acetylaminophenyl)-3-oxopropionate (14.7 g, 59.3 mmol) obtained in Example 33-(1) in methanol (150 ml) at 0° C. After stirred at 0° C. for 10 minutes, the reaction was stopped with 5% KHSO₄, and the solvent was distilled off. The residue was extracted with ethyl acetate-tetrahydrofuran (1:1, 100 ml) 3 times, and washed with an aqueous saturated sodium bicarbonate solution and saturated brine. This was dried with sodium sulfate, and the silica gel column chromatography [eluent: hexane-ethyl acetate (2.1)] and recrystallization from ethyl acetate-hexane (1:1) to obtain ethyl 3-(4-acetylaminophenyl)-3-hydroxypropionate (11.2 g, 44.4 mmol, 75%) as colorless prisms.

mp.102-103° C.

IR νₘₐₓ (KBr) cm⁻¹: 3600-3200 (br, OH, NH), 1722, 1668 (C═O).

¹H-NMR (CDCl₃) δ: 1.266 (3H, t, J=7.2 Hz), 2.162 (3H, s), 2.62-2.80 (2H, m), 4.181 (2H, q, J=7.2 Hz), 5.093 (1H, dd, J=5.2, 7.8 Hz), 7.312 (2H, d, J=8.4 Hz), 7.393 (1H, br), 7.466 (2H, d, J=8.4 Hz).

Elemental analysis (C₁₃H₁₇NO₄) Cal'd: C, 62.14; H, 6.82; N, 5.57 Found: C, 62.20; H, 6.77; N, 5.66.

(3) A mixture of ethyl 3-(4-acetylaminophenyl)-3-hydroxypropionate (11.2 g, 44.6 mmol) obtained in Example 33-(2), triethylamine (5.4 g, 53.6 mmol), methanesulfonyl chloride (5.6 g, 49.1 mmol) and ethyl acetate (100 ml) was stirred at 0° C. for 30 minutes. 1,8-diazabicyclo[5.4.0]-7-undecene (7.5 g, 49.1 mmol) was added to this solution. The mixture was stirred at 0° C. for 30 minutes. This mixture was diluted with ethyl acetate (100 ml), and washed with a 5% aqueous potassium hydrogen sulfate solution, an aqueous saturated sodium bicarbonate solution and saturated brine. After dried with sodium sulfate, the mixture was concentrated under reduced pressure. The residue was purified by silica gel column chromatography [eluent: hexane-ethyl acetate (1:1)] and recrystallization from ethyl acetate-hexane (1:1) to obtain ethyl 3-(4-acetylaminophenyl)-2-propenoate (8.0 g, 34.3 mmol, 77%) as colorless prisms.

mp.126-127° C.

IR νₘₐₓ (KBr) cm⁻¹: 3308 (NH), 1793, 1674 (C═O), 1633 (C═C).

¹H-NMR (CDCl₃) δ: 1.335 (3H, t, J=7.0 Hz), 2.196 (3H, s), 4.261 (2H, q, J=7.0 Hz), 6.362 (1H, d, J=16.2 Hz), 7.474 (2H, d, J=8.4 Hz), 7.556 (2H, d, J=8.4 Hz), 7.631 (1H, d, J=16.2 Hz).

Elemental analysis (C₁₃H₁₅NO₃) Cal'd: C, 66.94; H, 6.48; N, 6.00 Found: C, 66.97; H, 6.36; N, 6.16.

(4) 10% palladium carbon (0.7 g) was added to a solution of ethyl 3-(4-acetylaminophenyl)-2-propenoate (7.8 g, 33.4 mmol) obtained in Example 33-(3) in ethanol (100 ml). Normal pressure catalytic reduction was carried out at room temperature. The catalyst was filtered to remove, and the filtrate was concentrated under reduced pressure. The residue was purified by recrystallization from ethyl acetate-hexane (1:10) to obtain ethyl 3-(4-acetylaminophenyl)propionate (8.3 g, 35.3 mmol, 100%) as colorless prisms.

mp.52-53° C.

IR νₘₐₓ (KBr) cm⁻¹: 3308 (NH), 1732, 1666 (C═O).

¹H-NMR (CDCl₃) δ: 1.233 (3H, t, J=7.4 Hz), 2.156 (3H, s), 2.588 (2H, t, J=7.4 Hz), 2.910 (2H, t, J=7.4 Hz), 4.121 (2H, q, J=7.4 Hz), 7.146 (2H, d, J=8.4 Hz), 7.32-7.46 (1H, br), 7.408 (2H, d, J=8.4 Hz).

Elemental analysis (C₁₃H₁₇NO₃) Cal'd: C, 66.36; H, 7.28; N, 5.95 Found: C, 66.28; H, 7.31; N, 5.99.

(5) A mixture of ethyl 3-(4-acetylaminophenyl)propionate (8.0 g, 34.0 mmol) obtained in Example 33-(4), concentrated hydrochloric acid (30 ml) and ethanol (30 ml) was heated to reflex for 2 hours. The reaction solution was concentrated, and the residue was purified by recrystallization from ethyl acetate-hexane (1:1) to obtain ethyl 3-(4-aminophenyl)propionate hydrochloride (4.0 g, 17.4 mmol, 51%) as colorless prisms.

mp.143-153° C.

IR νₘₐₓ (KBr) cm⁻¹: 3200-2400 (br, NH₃+), 1726 (C═O).

¹H-NMR (D₂O) δ: 0.823 (3H, t, J=7.2 Hz), 2.389 (2H, t, J=7.2 Hz), 2.653 (2H, t, J=7.2 Hz), 3.753 (2H, q, J=7.2 Hz), 6.988 (2H, d, J=8.8 Hz), 7.069 (2H, d, J=8.8 Hz).

Elemental analysis (C₁₁H₁₆NO₂Cl) Cal'd: C, 57.52; H, 7.02; N, 6.10 Found: C, 57.43; H, 6.75; N, 6.19.

(6) Thionyl chloride (0.34 g, 2.81 mmol) was added to a solution of (3R,5S)-1-(3-acetoxy-2.2-dimethylpropyl)-7-chloro-5-(2,3-dimethoxyphenyl)-2-oxo-1,2,3,5-tetrahydro-4,1-benzoxazepine-3-acetic acid (0.5 g, 0.962 mmol) obtained in Example 1-(1) and N,N-dimethylformamide (0.02 ml) in tetrahydrofuran (5 ml) at room temperature. After stirred for 1 hour, the mixture was concentrated under reduced pressure. The residue was dissolved in tetrahydrofuran (3 ml), which was added to a mixture of ethyl 3-(4-aminophenyl)propionate hydrochloride (0.24 g, 1.06 mmol) obtained in Example 33-(5), triethylamine (0.24 g, 2.41 mmol) and tetrahydrofuran (3 ml). This was stirred at room temperature for 30 minutes, water was added, and tetrahydrofuran was distilled off. The residue was diluted with ethyl acetate (50 ml). This was washed with 1N hydrochloric acid and saturated brine, dried with sodium sulfate, and concentrated under reduced pressure. The residue was purified by silica gel column chromatography [eluent:ethyl acetate-hexane (3:4)] to obtain ethyl 3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate (0.51 g, 0.734 mmol, 76%) as a colorless amorphous powder.

[α]_{D}²²−128.5° (c=0.20, methanol).

IR νₘₐₓ (KBr) cm⁻¹: 3327 (NH), 1732, 1682 (C═O).

¹H-NMR (CDCl₃) δ: 0.954 (3H, s), 1.018 (3H, s), 1.238 (3H, t, J=7.2 Hz), 2.022 (3H, s), 2.584 (2H, t, J=7.2 Hz), 2.807 (1H, dd, J=5.2, 13.8 Hz), 2.912 (2H, t, J=7.2 Hz), 2.988 (1H, dd, J=7.2, 13.8 Hz), 3.530 (1H, d, J=13.8 Hz), 3.616 (3H, s), 3.727 (1H, d, J=11.4 Hz), 3.872 (1H, d, J=11.4 Hz), 3.892 (3H, s), 4.123 (2H, q, J=7.2 Hz), 4.405 (1H, dd, J=5.2, 7.2 Hz), 4.555 (1H, d, J=13.8 Hz), 6.295 (1H, s), 6.645 (1H, d, J=2.0 Hz), 6.97-7.43 (9H, m), 7.823 (1H, s).

Elemental analysis (C₃₇H₄₃N₂O₉Cl.0.3H₂O) Cal'd: C, 63.43; H, 6.27; N, 4.00 Found: C, 63.39; H, 6.09; N, 3.95.

(7) A mixture of ethyl 3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate (0.4 g, 0.575 mmol) obtained in Example 33-(6), a 1N aqueous sodium hydroxide solution (1.5 ml) and ethanol (5 ml) was stirred at 60° C. for 30 minutes. This was diluted with water (50 ml) and, after acidification, extracted with ethyl acetate (50 ml×2). This was washed with saturated brine, dried with sodium sulfate, and concentrated under reduced pressure. The residue was purified by silica gel column chromatography (eluent:ethyl acetate) and recrystallization from ethyl acetate-hexane (1:2) to obtain 3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic acid (0.16 g, 0.256 mmol, 45%) as colorless prisms.

mp.144-146° C.

[α]_{D}²²−124.5° (c=0.16, methanol).

IR νₘₐₓ (KBr) cm⁻¹: 3600-2400 (br, COOH, OH, NH), 1724, 1689, 1655 (C═O). ¹H-NMR (CDCl₃) δ: 0.647 (3H, s), 1.039 (3H, s), 2.643 (2H, t, J=7.4 Hz), 2.824 (1H, dd, J=5.8, 14.4 Hz), 2.918 (2H, t, J=7.4 Hz), 3.009 (1H, dd, J=7.4, 14.4 Hz), 3.167 (1H, d, J=11.6 Hz), 3.369 (1H, d, J=13.8 Hz), 3.607 (3H, s), 3.614 (1H, d, J=11.6 Hz), 3.890 (3H, s), 4.40-4.49 (2H, m), 6.184 (1H, s), 6.612 (1H, s), 6.96-7.44 (9H, m), 7.907 (1H, s).

Elemental analysis (C₃₃H₃₇N₂O₈Cl.1.5H₂O) Cal'd: C, 60.78; H, 6.18; N, 4.30 Found: C, 60.65; H, 6.02; N, 4.18.

### EXAMPLE 34

3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic Acid

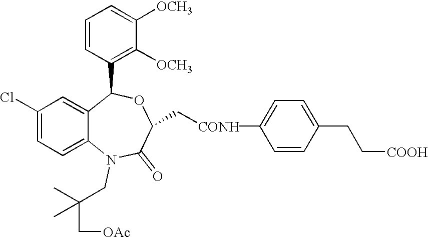

Acetyl chloride (2.0 g, 25.2 mmol) was added to a mixture of 3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic acid (4.5 g, 7.20 mmol) obtained in Example 33-(7), pyridine (2.6 g, 32.4 mmol) and ethyl acetate (50 ml). After stirred at room temperature for 3 hours, water (40 ml) was added to this mixture, and further stirred overnight. The organic layer was separated, and washed with 1N hydrochloric acid and saturated brine. This was dried with sodium sulfate, and concentrated under reduced pressure. The residue was purified by silica gel column chromatography [eluent:hexane-acetone-ethyl acetate (3:1.5:0.1)] to obtain 3-[4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic acid (3.2 g, 4.68 mmol, 65%) as a colorless amorphous powder.

[α]_{D}²²−124.7° (c=0.25, methanol).

IR νₘₐₓ (KBr) cm⁻¹: 3323 (br, NH), 3600-2400 (br, COOH), 1732, 1682 (C═O). ¹H-NMR (CDCl₃) δ: 0.936 (3H, s), 0.991 (3H, s), 1.998 (3H, s), 2.643 (2H, t, J=7.0 Hz), 2.813 (1H, dd, J=5.4, 14.0 Hz), 2.914 (2H, t, J=7.0 Hz), 3.034 (1H, dd, J=7.4, 14.0 Hz), 3.510 (1H, d, J=13.8 Hz), 3.608 (3H, s), 3.709 (1H, d, J=10.8 Hz), 3.844 (1H, d, J=10.8 Hz), 3.887 (3H, s), 4.438 (1H, dd, J=5.4, 7.4 Hz), 4.522 (1H, d, J=13.8 Hz), 6.282 (1H, s), 6.642 (1H, d, J=2.2 Hz), 6.96-7.52 (9H, m), 8.193 (1H, br).

### EXAMPLE 35

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic Acid

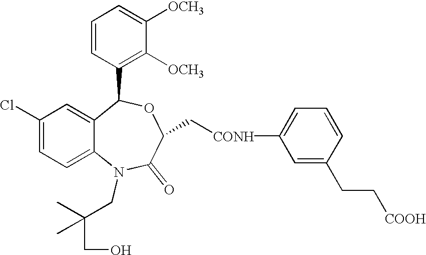

(1) Method A: 10% palladium carbon (0.5 g) was added to a solution of ethyl 3-(3-nitrophenyl)-2-propenoate (10 g, 45.2 mmol) in ethanol (200 ml), the mixture was subjected to normal pressure catalytic reduction at room temperature for 12 hours under hydrogen gas atmosphere. The catalyst was filtered to remove, and the filtrate was concentrated under reduced pressure. The residue was dissolved in ethyl acetate (100 ml), and a 4N solution of hydrogen chloride in ethyl acetate (15 ml) was added thereto. The solvent was distilled off, and the residue was washed with ethyl acetate-hexane (1:1) to obtain ethyl 3-(3-aminophenyl)propionate hydrochloride (10.4 g, 45.3 mmol, 100%) as colorless prisms.

Method B: 10% palladium carbon (2.5 g) was added to a solution of ethyl 3-(3-nitrophenyl)-2-propenoate (25 g, 0.113 mol) in ethanol (500 ml), and formic acid (29 g, 0.622 mol) was added dropwise. After stirred at room temperature for 6 hours, the catalyst was filtered to remove, and a 4N solution of hydrogen chloride in ethyl acetate (30 ml) was added to the filtrate. The solvent was distilled off, and the residue was washed with ethyl acetate-hexane (1:1) to obtain ethyl 3-(3-aminophenyl)propionate hydrochloride (24 g, 0.104 mol, 92%) as colorless prisms.

mp.124-131° C.

IR νₘₐₓ (KBr) cm⁻¹: 3200-2400 (br, NH₃⁺), 1726 (C═O).

¹H-NMR (D₂O) δ: 1.075 (3H, t, J=7.4 Hz), 2.643 (2H, t, J=7.4 Hz), 2.906 (2H, t, J=7.4 Hz), 4.002 (2H, q, J=7.4 Hz), 7.16-7.43 (4H, m).

(2) Thionyl chloride (0.67 g, 5.61 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to the mixture of ethyl 3-(3-aminophenyl)propionate hydrochloride (0.48 g, 2.11 mmol) obtained in Example 35-(1), triethylamine (0.24 g, 2.41 mmol) and tetrahydrofuran (5 ml). This was stirred at room temperature for 30 minutes, water was added, and tetrahydrofuran was distilled off. The residue was diluted with ethyl acetate (50 ml). This was washed with 1N hydrochloric acid, and an aqueous saturated sodium bicarbonate solution and saturated brine, dried with sodium sulfate, and concentrated under reduced pressure. The residue was purified by silica gel column chromatography [eluent:hexane-ethyl acetate (1:1)] to obtain ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate (1.2 g, 1.73 mmol, 90%) as a colorless amorphous powder.

[α]_{D}²²−123.1° (c=0.23, methanol).

IR νₘₐₓ (KBr) cm⁻¹: 3314 (NH), 1732, 1682 (C═O).

¹H-NMR (CDCl₃) δ: 0.958 (3H, s), 1.024 (3H, s), 1.236 (3H, t, J=7.0 Hz), 2.024 (3H, s), 2.603 (2H, t, J=7.4 Hz), 2.811 (1H, dd, J=6.2, 14.4 Hz), 2.927 (2H, t, J=7.4 Hz), 2.996 (1H, dd, J=7.4, 14.4 Hz), 3.538 (1H, d, J=14.2 Hz), 3.619 (3H, s), 3.732 (1H, d, J=11.4 Hz), 3.873 (1H, d, J=11.4 Hz), 3.894 (3H, s), 4.128 (2H, q, J=7.0 Hz), 4.410 (1H, dd, J=6.2, 7.4 Hz), 4.564 (1H, d, J=14.2 Hz), 6.301 (1H, s), 6.644 (1H, d, J=2.0 Hz), 6.93-7.39 (9H, m), 7.810 (1H, br).

Elemental analysis (C₃₇H₄₃N₂O₉Cl) Cal'd: C, 63.92; H, 6.23; N, 4.03. Found: C, 63.57; H, 6.52; N, 3.82

(3) Method C: A mixture of ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate (1.1 g, 1.58 mmol) obtained in Example 35-(2), a 1N aqueous sodium hydroxide solution (4 ml) and ethanol (10 ml) was stirred at 60° C. for 30 minutes. This was diluted with water (50 ml) and, after acidification, extracted with ethyl acetate (50 ml) 2 times. The extracts were combined, washed with saturated brine, dried with sodium sulfate, and concentrated under reduced pressure. The residue was purified by recrystallization from ethyl acetate-hexane (1:1) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid (1.0 g, 1.66 mmol, 100%) as colorless needles.

Method D: Triethylamine (2.0 g, 19.6 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (10 g, 19.2 mmol) obtained in Example 1-(1) in acetonitrile (60 ml) at room temperature. The mixture was ice-cooled, pivaloyl chloride (2.5 g, 21.1 mmol) was added dropwise over 10 minutes under nitrogen stream, and the mixture was stirred as it was under ice-cooling for 30 minutes. Ethyl 3-(3-aminophenyl)propionate hydrochloride (5.7 g, 24.8 mmol) obtained in Example 35-(1) was added, and triethylamine (4.3 g, 42.2 mmol) was added dropwise. A temperature was raised to room temperature, the mixture was stirred for 1 hour, and stirred at 60° C. for 3 hours. 1N hydrochloric acid (10 ml) was added, further water was added, and extracted with ethyl acetate (100 ml) 3 times. The whole organic layer was washed with saturated brine, dried with sodium sulfate, and concentrated under reduced pressure. The residue was dissolved in ethanol (80 ml), and a 1N aqueous sodium hydroxide solution (40 ml) was added. This was stirred at 60° C. for 30 minutes, diluted with water (50 ml) and, after acidification, extracted with ethyl acetate (80 ml) 2 times. The extracts were combined, washed with saturated brine, dried with sodium sulfate, and concentrated under reduced pressure. The residue was crystallized from ethyl acetate-hexane (1:1) and purified by recrystallization from ethanol-water (1:1) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid (8.5 g, 13.6 mmol. 71%) as colorless needles.

mp.141-144° C.

[α]_{D}²²−153.2° (c=0.48, methanol).

IR νₘₐₓ (KBr) cm⁻¹: 3600-2400 (br, COOH, OH, NH), 1714, 1651 (C═O).

¹H-NMR (CDCl₃) δ: 0.654 (3H, s), 1.048 (3H, s), 2.647 (2H, t, J=7.4 Hz), 2.826 (1H, dd, J=5.0, 14.6 Hz), 2.931 (2H, t, J=7.4 Hz), 3.007 (1H, dd, J=7.6, 14.6 Hz), 3.186 (1H, d, J=12.0 Hz), 3.387 (1H, d, J=14.6 Hz), 3.610 (3H, s), 3.624 (1H, d, J=12.0 Hz), 3.890 (3H, s), 4.40-4.51 (2H, m), 6.183 (1H, s), 6.624 (1H, d, J=1.8 Hz), 6.93-7.38 (9H, m), 7.945 (1H, br).

Elemental analysis (C₃₃H₃₇N₂O₈Cl) Cal'd: C, 63.41; H, 5.97; N, 4.48. Found: C, 63.18; H, 6.11; N, 4.36.

### EXAMPLE 36

3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic Acid

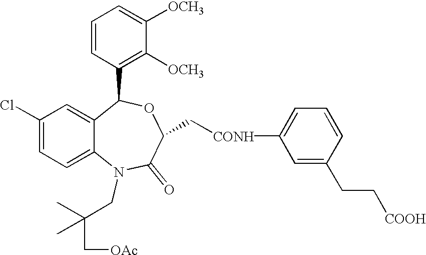

Acetyl chloride (0.22 g, 2.80 mmol) was added to the mixture of 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid (0.5 g, 0.800 mmol) obtained in Example 35-(3), pyridine (0.28 g, 3.60 mmol) and ethyl acetate (5 ml). After stirred at room temperature for 1 hour, water (4 ml) was added to this mixture, and the mixture was further stirred at room temperature for 3 hours. The organic layer was separated, and washed with 1N hydrochloric acid and saturated brine. This was dried with sodium sulfate, and concentrated under reduced pressure to obtain 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionic acid (0.41 g, 0.615 mmol, 77%) as a colorless amorphous powder.

[α]_{D}²²−124.9° (c=0.15, methanol).

IR νₘₐₓ (KBr) cm⁻¹: 3400-2400 (br, COOH, NH), 1732, 1668 (C═O).

¹H-NMR (CDCl₃) δ: 0.948 (3H, s), 1.012 (3H, s), 2.011 (3H, s), 2.644 (2H, t, J=7.6 Hz), 2.821 (1H, dd, J=5.4, 13.8 Hz), 2.919 (2H, t, J=7.6 Hz), 3.027 (1H, dd, J=8.0, 13.8 Hz), 3.534 (1H, d, J=14.2 Hz), 3.615 (3H, s), 3.735 (1H, d, J=11.0 Hz), 3.870 (1H, d, J=11.0 Hz), 3.890 (3H, s), 4.430 (1H, dd, J=5.4, 8.0 Hz), 4.550 (1H, d, J=14.2 Hz), 6.295 (1H, s), 6.647 (1H, d, J=1.4 Hz), 6.92-7.37 (9H, m), 8.099 (1H, br).

Elemental analysis (C₃₅H₃₉N₂O₉Cl.0.5H₂O) Cal'd: C, 62.17; H, 5.96; N, 4.14. Found: C, 62.37; H, 5.95; N, 4.08.

### EXAMPLE 37

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxyphenyl]propionic Acid

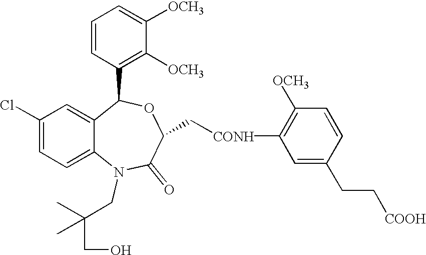

(1) A mixture of 4-methoxy-3-nitrobenzaldehyde (5 g, 27.6 mmol), (carboethoxymethylene)triphenylphosophine (11 g, 31.8 mmol) and tetrahydrofuran (50 ml) was stirred at 0° C. for 30 minutes. After further stirred at room temperature for 3 hours, this mixture was diluted with ethyl acetate (100 ml), and washed with 1N hydrochloric acid (15 ml), an aqueous saturated sodium bicarbonate solution and saturated brine. The mixture was dried with sodium sulfate, and concentrated under reduced pressure. The residue was purified by silica gel column chromatography [hexane-ethyl acetate (2:1)] and recrystallization from ethyl acetate-hexane (1:5) to obtain ethyl 3-(4-methoxy-3-nitrophenyl)-2-propenoate (5.12 g, 20.4 mmol, 75%) as a colorless powder. 10% palladium carbon (0.5 g) was added to a solution of the resulting ethyl 3-(4-methoxy-3-nitrophenyl)-2-propenoate in ethanol (100 ml), which was subjected to normal pressure catalytic reduction at room temperature for 5 hours. The catalyst was filtered to remove, and the filtrate was concentrated under reduced pressure. The residue was dissolved in ethyl acetate (50 ml), and a 4N solution of hydrogen chloride in ethyl acetate (10 ml) was added. The solvent was distilled off, and the residue was washed with ethyl acetate-hexane (1:1) to obtain ethyl 3-(amino-4-methoxyphenyl)propionate hydrochloride (5.07 g, 19.5 mmol, 96%) as colorless needles.

mp.156-161° C.

IR νₘₐₓ (KBr) cm⁻¹: 3200-2400 (br, NH₃+), 1724 (C═O).

¹H-NMR (D₂O) δ: 0.781 (3H, t, J=7.4 Hz), 2.314 (2H, t, J=7.4 Hz), 2.545 (2H, t, J=7.4 Hz), 3.534 (3H, s), 3.713 (2H, q, J=7.4 Hz), 6.755 (1H, d, J=8.6 Hz), 6.865 (1H, d, J=1.8 Hz), 6.945 (1H, dd, J=1.8, 8.6 Hz).

(2) Thionyl chloride (0.47 mg, 3.94 mmol) was added to a solution of (3R,5S)-1-(3-acetoxy-2-dimethylpropyl)-7-chloro-5-(2,3-dimethoxyphenyl)-2-oxo-1,2,3,5-tetrahydro-4,1-benzaldehyde-3-acetic acid (0.7 g, 1.35 mmol) obtained in Example 1-(1) and N,N-dimethylformamide (0.02 ml) in tetrahydrofuran (7 ml) at room temperature. After stirred for 1 hour, the mixture was concentrated under reduced pressure. The residue was dissolved in tetrahydrofuran (7 ml), and the solution was added to a mixture of ethyl 3-(3-amino-4-methoxyphenyl)propionate hydrochloride (0.39 g, 1.48 mmol) obtained in Example 37-(1), triethylamine (0.34 g, 3.38 mmol) and tetrahydrofuran (5 ml). This was stirred at room temperature for 30 minutes, water was added, and tetrahydrofuran was distilled off. The residue was diluted with ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxyphenyl]propionate (0.81 g, 1.12 mmol, 83%) as a colorless amorphous powder.

[α]_{D}²²−160.0° (c=0.31, methanol).

IR νₘₐₓ (KBr) cm⁻¹: 3344 (NH), 1732, 1682 (C═O).

¹H-NMR (CDCl₃) δ: 0.952 (3H, s), 1.020 (3H, s), 1.229 (3H, t, J=7.4 Hz), 2.026 (3H, s), 2.574 (2H, t, J=7.4 Hz), 2.80-2.90 (3H, m), 3.027 (1H, dd, J=6.2, 14.2 Hz), 3.545 (1H, d, J=13.8 Hz), 3.608 (3H, s), 3.720 (1H, d, J=11.4 Hz), 3.770 (3H, s), 3.870 (1H, d, J=11.4 Hz), 3.889 (3H, s), 4.113 (2H, q, J=7.4 Hz), 4.449 (1H, t, J=6.2 Hz), 4.579 (1H, d, J=13.8 Hz), 6.292 (1H, s), 6.636 (1H, s), 6.74-7.33 (7H, m), 8.16-8.22 (2H, m).

Elemental analysis (C₃₈H₄₅N₂O₁₀Cl) Cal'd: C, 62.93; H, 6.25; N, 3.86 Found: C, 62.71; H, 6.26; N, 3.76

(3) A mixture of ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxyphenyl]propionate (0.7 g, 0.965 mmol) obtained in Example 37-(2), a 1N aqueous sodium hydroxide solution (2 ml) and ethanol (7 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethanol-hexane (1:1) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxyphenyl]propionic acid (0.61 g, 0.931 mmol, 96%) as colorless needles.

[α]_{D}²²−172.8° (c=0.17, methanol).

IR νₘₐₓ (KBr) cm⁻¹: 3600-2400 (br, COOH, OH, NH), 1732, 1712, 1657 (C═O). ¹H-NMR (CDCl₃) δ: 0.648 (3H, s), 1.050 (3H, s), 2.625 (2H, t, J=7.4 Hz), 2.80-2.92 (3H, m), 3.066 (1H, dd, J=6.6, 14.6 Hz), 3.154 (1H, d, J=12.4 Hz), 3.388 (1H, d, J=14.2 Hz), 3.603 (3H, s), 3.616 (1H, d, J=12.4 Hz), 3.777 (3H, s), 3.890 (3H, s), 4.42-4.52 (2H, m), 6.186 (1H, s), 6.620 (1H, s), 6.75-7.36 (7H, m), 8.16-8.22 (2H, m).

Elemental analysis (C₃₄H₃₉N₂O₉Cl) Cal'd: C, 62.33; H, 6.00; N, 4.28 Found: C, 62.09; H, 6.11; N, 4.02

### EXAMPLE 38

3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxyphenyl]propionic Acid

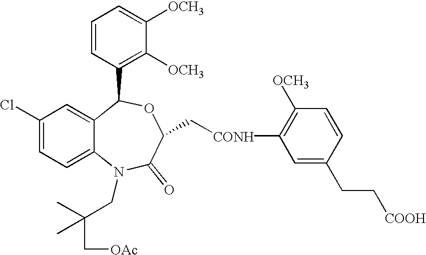

Acetyl chloride (0.13 g, 1.60 mmol) was added to a mixture of 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxyphenyl]propionic acid (0.3 g, 0.458 mmol) obtained in Example 37-(3), pyridine (0.16 g, 2.06 mmol) and ethyl acetate (3 ml). After stirred at room temperature for 2 hours, water (3 ml) was added to this mixture, and further stirred at room temperature for 3 hours. The organic layer was separated, and washed with 1N hydrochloric acid and saturated brine. This was dried with sodium sulfate, and concentrated under reduced pressure. The residue was purified by recrystallization from ethyl acetate-hexane (1:1) to obtain 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxyphenyl]propionic acid (0.23 g, 0.330 mmol, 72%) as colorless needles.

[α]_{D}²²−163.2° (c=0.15, methanol).

IR νₘₐₓ (KBr) cm⁻¹: 3400-2400 (br, COOH, NH), 1736, 1678 (C═O).

¹H-NMR (CDCl₃) δ: 0.952 (3H, s), 1.018 (3H, s), 2.024 (3H, s), 2.632 (2H, t, J=7.6 Hz), 2.80-2.92 (3H, m), 3.032 (1H, dd, J=6.6, 15.0 Hz), 3.544 (1H, d, J=14.0 Hz), 3.608 (3H, s), 3.719 (1H, d, J=11.2 Hz), 3.769 (3H, s), 3.871 (1H, d, J=11.2 Hz), 3.888 (3H, s), 4.443 (1H, t, J=6.6 Hz), 4.577 (1H, d, J=14.0 Hz), 6.292 (1H, s), 6.638 (1H, s), 6.74-7.33 (7H, m), 8.19-8.21 (2H, m).

Elemental analysis (C₃₆H₄₁N₂O₁₀Cl) Cal'd: C, 62.02; H, 5.93; N, 4.02 Found: C, 61.84; H, 6.17; N, 4.02

### EXAMPLE 39

3-[3-[[[(3R 5S)-7-chloro-5-(2,3-dimethoxyphenyl)-1-(3-hydroxy-2,2-dimethlpropyl)-2-oxo-1,2,3,5-tetrahydro-4,1-benzoxazepin-3-yl]acetyl]amino]-3-methylphenyl]propionic Acid

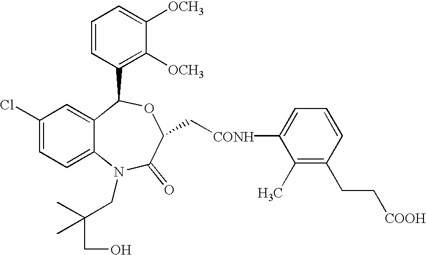

(1) Carbonyldiimidazole (9.8 g, 60.7 mmol) was added to a suspension of 2-methyl-3-nitrobenzoic acid (10 g, 55.2 mmol) in tetrahydrofuran (100 ml) at room temperature. After stirred at room temperature for 6 hours, a magnesium salt of malonic acid monoethyl ester (8.7 g, 30.4 mmol) was added to this mixture. The mixture was stirred at 60° C. for 3 hours and diluted with ethyl acetate (100 ml), washed with an aqueous saturated ammonium chloride solution 2 times, dried with sodium sulfate, and concentrated under reduced pressure. The residue was purified by silica gel column chromatography [eluent:hexane-ethyl acetate (3:1)] and recrystallization from hexane to obtain ethyl 3-(2-methyl-3-nitrophenyl)-3-oxopropionate (9.7 g, 38.7 mmol, 70%) as a colorless powder.

IR νₘₐₓ (KBr) cm⁻¹: 3500-3300 (br, OH), 1738, 1699 (C═O).

¹H-NMR (CDCl₃) δ: 1.233 (½×3H, t, J=7.0 Hz), 1.346 (½×3H, t, J=7.0 Hz), 2.549 (3H, s), 3.923 (½×2H, s), 4.186 (½×2H, q, J=7.0 Hz), 4.291 (½×3H, t, J=7.0 Hz), 5.275 (½×1H, s), 7.32-7.88 (3H, m).

Elemental analysis (C₁₂H₁₃NO₅) Cal'd: C, 57.37; H, 5.22; N, 5.58 Found: C, 57.15; H, 5.13; N, 5.65

(2) Sodium borohydride (1.5 g, 38.7 mmol) was added to a solution of ethyl 3-(2-methyl-3-nitrophenyl)-3-oxopropionate (9.7 g, 38.7 mmol) obtained in Example 39-(1) in ethanol (100 ml) at 0° C. After stirred at room temperature for 30 minutes, the mixture was diluted with ethyl acetate (300 ml), and washed with water, 1N hydrochloric acid, an aqueous saturated sodium bicarbonate solution and saturated brine. After dried with sodium sulfate, the residue was purified by silica gel column chromatography [eluent:hexane-ethyl acetate (2:1)] to obtain ethyl 3-(2-methyl-3-nitrophenyl)-3-hydroxypropionate (3.4 g, 13.4 mmol, 35%) as a colorless oil.

IR νₘₐₓ (KBr) cm⁻¹: 3600-3200 (br, OH), 1732 (C═O).

¹H-NMR (CDCl₃) δ: 1.293 (3H, t, J=7.0 Hz), 2.427 (3H, s), 2.63-2.68 (2H, m), 3.558 (1H, d, J=3.4 Hz), 4.223 (2H, q, J=7.0 Hz), 5.39-5.47 (1H, m), 7.368 (1H, t, J=8.0 Hz), 7.682 (1H, dd, J=1.2, 8.0 Hz), 7.809 (1H, d, J=8.0 Hz).

(3) A mixture of ethyl 3-(2-methyl-3-nitrophenyl)-3-hydroxypropionate (3.4 g, 13.4 mmol) obtained in Example 39-(2), triethylamine (1.6 g, 16.1 mmol), methanesulfonyl chloride (1.7 g, 14.7 mmol) and ethyl acetate (35 ml) was stirred at 0° C. for 30 minutes. 1,8-diazabicyclo[5.4.0]-7-undecene (2.2 g, 14.7 mmol) was added to this solution. This mixture was stirred at 0° C. for 30 minutes. This mixture was diluted with ethyl acetate (100 ml), and washed with a 5% aqueous potassium hydrogen sulfate solution, an aqueous saturated sodium bicarbonate solution and saturated brine. The mixture was dried with sodium sulfate, and concentrated under reduced pressure. The residue was purified by silica gel column chromatography [eluent:hexane-ethyl acetate (10:1)] and recrystallization from ethyl acetate-hexane (1:1) to obtain ethyl 3-(2-methyl-3-nitrophenyl)-2-propenoate (1:98 g, 8.42 mmol, 63%) as a colorless powder.

mp.53-55° C.

IR νₘₐₓ (KBr) cm⁻¹: 1714 (C═O), 1639 (C═C).

¹H-NMR (CDCl₃) δ: 1.355 (3H, t, J=7.0 Hz), 2.516 (3H, s), 4.296 (2H, q, J=7.0 Hz), 6.366 (1H, d, J=15.8 Hz), 7.351 (1H, d, J=8.0 Hz), 7.69-7.78 (2H, m), 7.970 (1H, d, J=15.8 Hz).

Elemental analysis (C₁₂H₁₃NO₄) Cal'd: C, 61.27; H, 5.57; N, 5.95. Found: C, 61.09; H, 5.44; N, 5.93.

(4) 10% palladium carbon (0.2 g) was added to a solution of ethyl 3-(2-methyl-3-nitrophenyl)-2-propenoate (1.9 g, 8.03 mmol) obtained in Example 39-(3) in ethanol (50 ml). Normal pressure catalytic reduction was performed at room temperature overnight. The catalyst was filtered to remove, and filtrate was concentrated under reduced pressure. The residue was dissolved in ethyl acetate (50 ml), and a 4N solution of hydrogen chloride in ethyl acetate (3 ml) was added. After concentration under reduced pressure, the residue was washed with diethyl ether-hexane (1:1) to obtain ethyl 3-(3-amino-2-methylphenyl)propionate hydrochloride (1.84 g, 7.55 mmol, 94%) as colorless plates.

mp.148-152° C.

IR νₘₐₓ (KBr) cm⁻¹: 3200-2400 (br, NH₃⁺), 1732 (C═O).

¹H-NMR (D₂O) δ: 1.051 (3H, t, J=7.2 Hz), 2.185 (3H, s), 2.555 (2H, t, J=7.4 Hz), 2.899 (2H, t, J=7.4 Hz), 3.975 (2H, q, J=7.2 Hz), 7.11-7.19 (3H, m).

Elemental analysis (C₁₂H₁₈NO₂Cl) Cal'd: C, 59.13; H, 7.44; N, 5.75. Found: C, 58.84; H, 7.31; N, 5.58.

(5) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of ethyl 3-(3-amino-2-methylphenyl)propionate hydrochloride (0.51 g, 2.11 mmol) obtained in Example 39-(4), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionate (1.0 g, 1.41 mmol, 73%) as a colorless amorphous powder.

[α]_{D}²²−154.8° (c=0.28, methanol).

IR νₘₐₓ (KBr) cm⁻¹: 3312 (NH), 1732, 1678 (C═O).

¹H-NMR (CDCl₃) δ: 0.963 (3H, s), 1.024 (3H, s), 1.255 (3H, t, J=7.0 Hz), 2.026 (3H, s), 2.167 (3H, s), 2.541 (2H, t, J=8.0 Hz), 2.828 (1H, d, J=5.2, 14.0 Hz), 2.959 (2H, t, J=8.0 Hz), 3.072 (1H, dd, J=7.6, 14.0 Hz), 3.539 (1H, d, J=13.8 Hz), 3.615 (3H, s), 3.723 (1H, d, J=11.4 Hz), 3.875 (1H, d, J=11.4 Hz), 3.892 (3H, s), 4.142 (2H, q, J=7.0 Hz), 4.419 (1H, dd, J=5.2, 7.6 Hz), 4.561 (1H, d, J=13.8 Hz), 6.297 (1H, s), 6.639 (1H, d, J=2.0 Hz), 6.96 -7.37 (7H, m), 7.56-7.67 (2H, m).

Elemental analysis (C₃₈H₄₅N₂O₉Cl) Cal'd: C, 64.35; H, 6.40; N, 3.95. Found: C, 64.15; H, 6.52; N, 3.74.

(6) A mixture of ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionate (1.0 g, 1.41 mmol) obtained in Example 39-(5), a 1N aqueous sodium hydroxide solution (3 ml) and ethanol (10 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by silica gel chromatography [ethyl acetate-methanol (10:1)] to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-methylphenyl]propionic acid (0.54 g, 0.845 mmol, 60%) as a colorless amorphous powder

[α]_{D}²²−165.1° (c=0.16, methanol).

IR νₘₐₓ (KBr) cm⁻¹: 3600-2400 (br, COOH, NH, OH), 1728, 1712, 1651 (C═O). ¹H-NMR (CDCl₃) δ: 0.658 (3H, s), 1.050 (3H, s), 2.169 (3H, s), 2.586 (2H, t, J=7.8 Hz), 2.848 (1H, d, J=5.0, 14.2 Hz), 2.971 (2H, t, J=7.8 Hz), 3.084 (1H, dd, J=4.2, 14.2 Hz), 3.184 (1H, d, J=12.0 Hz), 3.388 (1H, d, J=14.2 Hz), 3.614 (3H, s), 3.628 (1H, d, J=12.0 Hz), 3.892 (3H, s), 4.23-4.50 (2H, m), 6.198 (1H, s), 6.623 (1H, d, J=2.0 Hz), 6.95-7.40 (7H, m), 7.51-7.65 (2H, m).

Elemental analysis (C₃₄H₃₉N₂O₈Cl) Cal'd: C, 63.01; H, 6.22; N, 4.32. Found: C, 63.14; H, 6.33; N, 4.31.

### EXAMPLE 40

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionic Acid

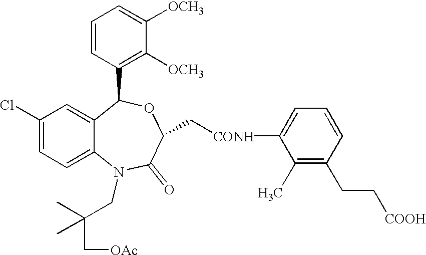

Acetyl chloride (0.10 g, 1.31 mmol) was added to a mixture of 3-[3-[[[(3R,5S)-7-chloro-5-(2,3-dimethoxyphenyl)-1-(3-hydoroxy-2,2-dimethylpropyl)-2-oxo-1,2,3,5-tetrahydro-4,1-benzoxazepin-3-yl]acetyl]amino]-2-methylphenyl]propionic acid (0.24 g, 0.376 mmol) obtained in Example 39-(6), pyridine (0.13 g, 1.69 mmol) and ethyl acetate (5 ml). After stirred at room temperature for 1 hour, water (4 ml) was added to this mixture, and the mixture was further stirred at room temperature for 1 hour. The organic layer was separated, and washed with 1N hydrochloric acid and saturated brine. This was dried with sodium sulfate, and concentrated under reduced pressure to obtain 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionic acid (0.18 g, 0.264 mmol, 70%) as a colorless amorphous powder.

[α]_{D}²²−141.1° (c=0.27, methanol).

IR νₘₐₓ (KBr) cm⁻¹: 3400-2400 (br, COOH, NH), 1732, 1682 (C═O).

¹H-NMR (CDCl₃) δ: 0.963 (3H, s), 1.018 (3H, s), 2.022 (3H, s), 2.156 (3H, s), 2.590 (2H, t, J=7.9 Hz), 2.838 (1H, d, J=4.4, 14.4 Hz), 2.967 (2H, t, J=7.9 Hz), 3.076 (1H, dd, J=8.0, 14.4 Hz), 3.538 (1H, d, J=14.2 Hz), 3.613 (3H, s), 3.725 (1H, d, J=11.4 Hz), 3.614 (3H, s), 3.879 (1H, d, J=11.4 Hz), 3.890 (3H, s), 4.425 (1H, dd, J=4.4, 8.0 Hz), 4.559 (1H, d, J=14.2 Hz), 6.297 (1H, s), 6.643 (1H, s), 6.96-7.32 (7H, m), 7.54-7.76 (2H, m).

Elemental Analysis (C₃₆H₄₁N₂O₉Cl) Cal'd: C, 63.48; H, 6.07; N, 4.11. Found: C, 63.16; H, 6.40; N, 3.75.

### EXAMPLE 41

3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-methylphenyl]propionic Acid

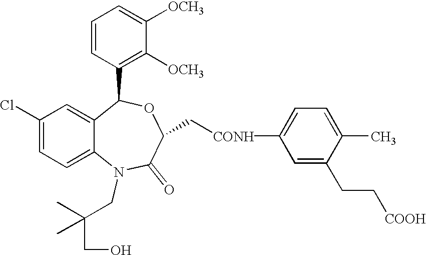

(1) Carbonyldiimidazole (4.9 g, 30.4 mmol) was added to a solution of 2-methyl-5-nitrobenzoic acid (5 g, 27.6 mmol) in tetrahydrofuran (50 ml) at room temperature. After stirred at room temperature for 6 hours, a magnesium salt of malonic acid monoethyl ester (4.4 g, 15.2 mmol) was added. This mixture was stirred at 60° C. for 1.5 hours, the reaction solution was diluted with ethyl acetate (100 ml), and washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (4:1)] to obtain ethyl 3-(2-methyl-5-nitrophenyl)-3-oxopropionate (5.4 g, 21.5 mmol, 78%) as a colorless oil.

IR νₘₐₓ (KBr) cm⁻¹: 3100-2600 (br, OH), 1741, 1699 (C═O).

¹H-NMR (CDCl₃) δ: 1.264 (⅗×3H, t, J=7.0 Hz), 1.354 (⅖×3H, t, J=7.0 Hz), 2.572 (⅖×3H, s), 2.647 (⅗×3H, s), 4.017 (⅗×2H, s), 4.213 (⅗×2H, q, J=7.0 Hz), 4.297 (⅖×2H, q, J=7.0 Hz), 5.361 (⅖×1H, s), 7.38-8.52 (3H, m).

(2) Sodium borohydride (0.98 g, 25.8 mmol) was added to a solution of ethyl 3-(2-methyl-5-nitrophenyl)-3-oxopropionate (5.4 g, 21.5 mmol) obtained in Example 41-(1) in ethanol (50 ml) at −78° C. After stirred at −78° C. for 30 minutes, 1N hydrochloric acid (30 ml) was added. this mixture was diluted with ethyl acetate (200 ml), washed with water, aqueous saturated sodium bicarbonate solution and saturated brine, dried with sodium sulfate, and the residue was purified by silica gel column chromatography [eluent:hexane-ethyl acetate (2:1)] to obtain ethyl 3-(2-methyl-5-nitrophenyl)-3-hydroxypropionate (4.7 g, 18.6 mmol, 86%) as a colorless oil.

IR νₘₐₓ (KBr) cm⁻¹: 3600-3200 (br, OH), 1732 (C═O).

¹H-NMR (CDCl₃) δ: 1.299 (3H, t, J=7.2 Hz), 2.433 (3H, s), 2.680 (2H, d, J=6.2 Hz), 3.602 (1H, d, J=3.2 Hz), 4.231 (2H, q, J=7.2 Hz), 5.371 (1H, dt, J=3.2, 6.2 Hz), 7.306 (1H, d, J=8.4 Hz), 8.043 (1H, dd, J=2.6, 8.4 Hz), 8.241 (1H, d, J=2.6 Hz).

(3) A mixture of ethyl 3-(2-methyl-5-nitrophenyl)-3-hydroxypropionate (4.5 g, 17.8 mmol) obtained in Example 41-(2), triethylamine (2.2 g, 21.4 mmol), methanesulfonyl chloride (2.2 g, 19.6 mmol) and ethyl acetate (50 ml) was stirred at 0° C. for 30 minutes. 1,8-Diazabicyclo[5.4.0]-7-undecene (3.9 g, 19.6 mmol) was added, and this mixture was stirred at 0° C. for 30 minutes. This mixture was diluted with ethyl acetate (100 ml), and washed with 1N hydrochloric acid, (40 ml) an aqueous saturated sodium bicarbonate solution and saturated brine. The mixture was dried with sodium sulfate, and concentrated under reduce pressure. The residue was purified by recrystallization form ethyl acetate-hexane (1:2) to obtain ethyl 3-(2-methyl-5-nitrophenyl)-2-propenoate (3.1 g, 13.2 mmol, 74%) as colorless prisms.

mp.93-95° C.

IR νₘₐₓ (KBr) cm⁻¹: 1716, 1705 (C═O), 1635 (C═C).

¹H-NMR (CDCl₃) δ: 1.361 (3H, t, J=7.2 Hz), 2.535 (3H, s), 4.301 (2H, q, J=7.2 Hz), 6.502 (1H, d, J=15.8 Hz), 7.381 (1H, d, J=8.4 Hz), 7.917 (1H, d, J=15.8 Hz), 8.114 (1H, dd, J=2.2, 8.4 Hz), 8.401 (1H, d, J=2.2 Hz).

Elemental Analysis (C₁₂H₁₃NO₄.0.2H₂O) Cal'd: C, 60.35; H, 5.65; N, 5.86. Found: C, 60.42; H, 5.49; N, 5.77.

(4) 10% palladium carbon (0.2 g) was added to a solution of ethyl 3-(2-methyl-5-nitrophenyl)-2-propenoate (2.9 g, 12.3 mmol) obtained in Example 41-(3) in ethanol (60 ml). This suspension was subjected to normal pressure catalytic reduction at room temperature for 4 hours. The catalyst was filtered to remove, and the filtrate was concentrated under reduced pressure. The residue was diluted with ethyl acetate (50 ml), and a 4N solution of hydrogen chloride in ethyl acetate (5 ml) was added. The solvent was distilled off, and residue was washed with ethyl acetate-Et₂O (1:1) to obtain ethyl 3-(5-amino-2-methylphenyl)propionate hydrochloride (2.7 g, 11.1 mmol, 90%) as colorless prisms.

mp.135-142° C.

IR νₘₐₓ (KBr) cm⁻¹: 3200-2400 (br, NH₃⁺), 1720 (C═O).

¹H-NMR (D₂O) δ: 1.037 (3H, t, J=7.2 Hz), 2.198 (3H, s), 2.551 (2H, t, J=7.4 Hz), 2.846 (2H, t, J=7.4 Hz), 3.969 (2H, q, J=7.2 Hz), 6.99-7.22 (3H, m).

Elemental Analysis (C₁₂H₁₈NO₂Cl.0.1H₂O) Cal'd: C, 58.70; H, 7.47; N, 5.70. Found: C, 58.61; H, 7.59; N, 5.62.

(5) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of ethyl 3-(5-amino-2-methylphenyl)propionate hydrochloride (0.51 g, 2.11 mmol) obtained in Example 41-(4), triethylamine (0.48 g, 4.80 mmol) and tetrahydrofuran (10 ml). This was stirred at room temperature for 30 minutes, water was added, and tetrahydrofuran was distilled off. The residue was diluted ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-methylphenyl]propionate (1.2 g, 1.69 mmol, 88%) as a colorless amorphous powder.

[α]_{D}²²−135.3° (c=0.20, methanol).

IR νₘₐₓ (KBr) cm⁻¹: 3327 (NH), 1732, 1682 (C═O).

¹H-NMR (CDCl₃) δ: 0.958 (3H, s), 1.024 (3H, s), 1.251 (3H, t, J=7.2 Hz), 2.024 (3H, s), 2.275 (3H, s), 2.550 (2H, t, J=8.8 Hz), 2.798 (1H, d, J=5.8, 13.8 Hz), 2.909 (2H, t, J=8.8 Hz), 2.982 (1H, dd, J=7.0, 13.8 Hz), 3.535 (1H, d, J=14.0 Hz), 3.618 (3H, s), 3.730 (1H, d, J=11.0 Hz), 3.869 (1H, d, J=11.0 Hz), 3.892 (3H, s), 4.143 (2H, q, J=7.2 Hz), 4.411 (1H, dd, J=5.8, 7.0 Hz), 4.560 (1H, d, J=14.0 Hz), 6.296 (1H, s), 6.639 (1H, d, J=2.0 Hz), 6.96-7.33 (8H, m), 7.56-7.67 (1H, m).

Elemental Analysis (C₃₈H₄₅N₂O₉Cl) Cal'd: C, 64.35; H, 6.40; N, 3.95. Found: C, 64.03; H, 6.50; N, 3.78.

(6) A mixture of ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-methylphenyl]propionate (1.1 g, 1.55 mmol) obtained in Example 41-(5), a 1N aqueous sodium hydroxide solution (5 ml) and ethanol (10 ml) was stirred at 60° C. for 30 minutes. This was diluted with water (50 ml) and, after acidification, extracted with ethyl acetate (50 ml×2). This was washed with saturated brine, dried with sodium sulfate, and concentrated under reduced pressure. The residue was purified by recrystallization from ethyl acetate-hexane (1:1) to obtain 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-methylphenyl]propionic acid (0.62 g, 0.970 mmol, 63%) as colorless needles.

[α]_{D}²²−149.1° (c=0.14, methanol).

IR νₘₐₓ (KBr) cm⁻¹: 3600-2400 (br, COOH, NH, OH), 1716, 1658 (C═O).

¹H-NMR (CDCl₃) δ: 0.652 (3H, s), 1.044 (3H, s), 2.265 (3H, s), 2.599 (2H, t, J=7.8 Hz), 2.811 (1H, d, J=5.4, 14.2 Hz), 2.914 (2H, t, J=7.8 Hz), 2.998 (1H, dd, J=7.2, 14.2 Hz), 3.187 (1H, d, J=11.8 Hz), 3.383 (1H, d, J=14.6 Hz), 3.606 (3H, s), 3.623 (1H, d, J=11.8 Hz), 3.888 (3H, s), 4.39-4.50 (2H, m), 6.174 (1H, s), 6.620 (1H, d, J=2.0 Hz), 6.965-7.40 (8H, m), 7.912 (1H, br).

Elemental Analysis (C₃₄H₃₉N₂O₈Cl.0.7H₂O) Cal'd: C, 62.66; H, 6.25; N, 4.30. Found: C, 62.66; H, 6.58; N, 4.05.

### EXAMPLE 42

3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionic Acid

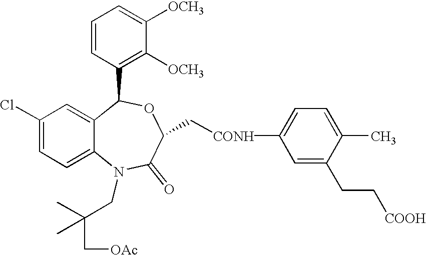

Acetyl chloride (0.13 g, 1.64 mmol) was added to a mixture of 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-methylphenyl]propionic acid (0.3 g, 0.469 mmol) obtained in Example 41-(6), pyridine (0.17 g, 2.11 mmol) and ethyl acetate (5 ml). After stirred at room temperature for 1 hour, water (4 ml) was added to this mixture, and the mixture was further stirred at room temperature for 1 hour. The organic layer was separated, and washed with 1N hydrochloric acid and saturated brine. This was dried with sodium sulfate, and concentrated under reduced pressure to obtain 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylphenyl]propionic acid (0.33 g, 0.484 mmol, 100%) as a colorless amorphous powder.

[α]_{D}²²−132.9° (c=0.20, methanol).

IR νₘₐₓ (KBr) cm⁻¹: 3400-2400 (br, COOH, NH), 1732, 1668 (C═O).

¹H-NMR (CDCl₃) δ: 0.943 (3H, s), 1.011 (3H, s), 2.006 (3H, s), 2.260 (3H, s), 2.584 (2H, t, J=7.2 Hz), 2.811 (1H, d, J=5.4, 14.0 Hz), 2.894 (2H, t, J=7.2 Hz), 3.028 (1H, dd, J=7.4, 14.4 Hz), 3.531 (1H, d, J=14.0 Hz), 3.614 (3H, s), 3.732 (1H, d, J=11.4 Hz), 3.866 (1H, d, J=11.4 Hz), 3.886 (3H, s), 4.434 (1H, dd, J=5.4, 7.4 Hz), 4.541 (1H, d, J=14.0 Hz), 6.288 (1H, s), 6.637 (1H, s) 6.97-7.33 (8H, m), 8.079 (1H, br).

Elemental Analysis (C₃₆H₄₁N₂O₉Cl.0.5H₂O) Cal'd: C, 62.65; H, 6.13; N, 4.06. Found: C, 62.60; H, 6.16; N, 3.81.

### EXAMPLE 43

3-[[[(3R,5S)-7-chloro-5-(2,3-dimethoxyphenyl)-1-(3-hydoroxy-2-dimethylpropyl)-2-oxo-1,2,3,5-tetrahydro-4,1-benzoxazepin-3-yl]acetyl]amino]-2-methylbenzoic acid

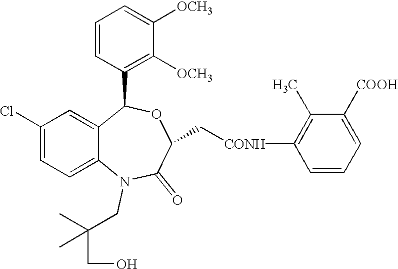

(1) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of methyl 3-amino-2-methylbenzoate hydrochloride (0.43 g, 2.11 mmol), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid, an aqueous saturated sodium bicarbonate solution and saturated brine, dried with sodium sulfate, and concentrate under reduced pressure. The residue was purified by silica gel column chromatography [eluent:hexane-ethyl acetate (1:1)] to obtain methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylbenzoate (0.50 g, 0.749 mmol, 39%) as a colorless amorphous powder.

[α]_{D}²²−134.3° (c=0.16, methanol).

IR νₘₐₓ (KBr) cm⁻¹: 3400-3200 (br, NH), 1724, 1682 (C═O).

¹H-NMR (CDCl₃) δ: 0.960 (3H, s), 1.020 (3H, s), 2.028 (3H, s), 2.414 (3H, s), 2.843 (1H, dd, J=5.0, 14.0 Hz), 3.100 (1H, dd, J=7.6, 14.0 Hz), 3.540 (1H, d, J=14.2 Hz), 3.618 (3H, s), 3.717 (1H, d, J=11.0 Hz), 3.873 (1H, d, J=11.0 Hz), 3.890 (6H, s), 4.383 (1H, dd, J=5.0, 7.6 Hz), 4.565 (1H, d, J=14.2 Hz), 6.297 (1H, s), 6.650 (1H, d, J=1.8 Hz), 6.96-7.38 (6H, m), 7.625 (1H, d, J=8.0 Hz), 7.865 (1H, br), 7.938 (1H, d, J=7.8 Hz).

Elemental Analysis (C₃₅H₃₉N₂O₉Cl) Cal'd: C, 63.01; H, 5.89; N, 4.20. Found: C, 62.73; H, 5.94; N, 4.16.

(2) A mixture of methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylbenzoate (0.4 g, 0.60 mmol) obtained in Example 43-(1), a 1N aqueous sodium hydroxide solution (1.5 ml) and ethanol (4 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethyl acetate to obtain 3-[[[(3R,5S)-7-chloro-5-(2,3-dimethoxyphenyl)-1-(3-hydroxy-2-dimethylpropyl)-2-oxo-1,2,3,5-tetrahydro-4,1-benzoxazepin-3-yl]acetyl]amino]-2-methylbenzoic acid (0.16 mg, 0.262 mmol, 44%) as colorless prisms.

mp.165-168° C.

[α]_{D}²²−149.6° (c=0.21, methanol).

IR νₘₐₓ (KBr) cm⁻¹: 3600-2400 (br, COOH, OH, NH), 1651 (C═O).

¹H-NMR (CDCl₃) δ: 0.663 (3H, s), 1.057 (3H, s), 2.491 (3H, s), 2.874 (1H, dd, J=5.2, 14.4 Hz), 3.131 (1H, dd, J=8.4, 14.4 Hz), 3.199 (1H, d, J=11.4 Hz), 3.399 (1H, d, J=14.2 Hz), 3.615 (3H, s), 3.639 (1H, d, J=11.4 Hz), 3.894 (3H, s), 4.42-4.52 (2H, m), 6.203 (1H, s), 6.635 (1H, d, J=1.8 Hz), 6.97-7.36 (6H, m), 7.77-7.93 (3H, m).

Elemental Analysis (C₃₂H₃₅N₂O₈Cl.0.2H₂O) Cal'd: C, 62.53; H, 5.80; N, 4.56. Found: C, 62.45; H, 5.89; N, 4.35.

### EXAMPLE 44

3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-metylbenzoic acid

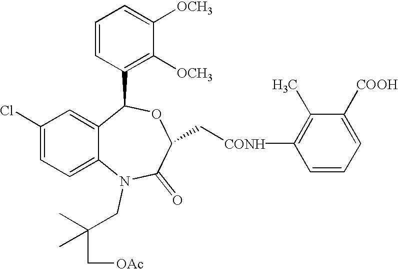

Acetyl chloride (36 mg, 0.458 mmol) was added to a mixture of 3-[[[(3R,5S)-7-chloro-5-(2,3-dimethoxyphenyl)-1-(3-hydroxy-2-dimethylpropyl)-2-oxo-1,2,3,5-tetrahydro-4,1-benzoxazepin-3-yl]acetyl]amino]-2-methylbenzoic acid (80 mg, 0.131 mmol) obtained in Example 43-(2), pyridine (47 mg, 0.589 mmol) and ethyl acetate (2 ml). After stirred at room temperature for 1 hour, water (4 ml) was added to this mixture, and the mixture was further stirred at room temperature for 3 hours. The organic layer was separated, and washed with 1N hydrochloric acid and saturated brine. This was dried with sodium sulfate, and concentrated under reduced pressure. The residue was purified by recrystallization form ethyl acetate-hexane (1:1) to obtain 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-methylbenzoic acid (85 mg, 0.130 mmol, 99%) as a colorless powder.

mp.139-142° C.

[α]_{D}²²−143.2° (c=0.17, methanol).

IR νₘₐₓ (KBr) cm⁻¹: 3400-2400 (br, COOH, NH), 1728, 1682 (C═O).

¹H-NMR (CDCl₃) δ: 0.967 (3H, s), 1.024 (3H, s), 2.027 (3H, s), 2.474 (3H, s), 2.868 (1H, dd, J=5.2, 14.8 Hz), 3.121 (1H, dd, J=7.6, 14.8 Hz), 3.551 (1H, d, J=14.2 Hz), 3.621 (3H, s), 3.728 (1H, d, J=11.2 Hz), 3.881 (1H, d, J=11.2 Hz), 3.894 (3H, s), 4.422 (1H, dd, J=5.2, 7.6 Hz), 4.576 (1H, d, J=14.2 Hz), 6.308 (1H, s), 6.656 (1H, s), 6.97-7.38 (6H, m), 7.790 (1H, d, J=7.4 Hz), 7.979 (1H, s), 7.967 (1H, d, J=7.4 Hz).

Elemental Analysis (C₃₄H₃₇N₂O₉Cl.0.5H₂O) Cal'd: C, 61.68; H, 5.78; N, 4.23. Found: C, 61.85; H, 5.87; N, 4.03.

### EXAMPLE 45

3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylbenzoic acid

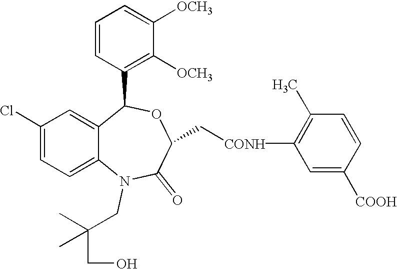

(1) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of methyl 3-amino-4-methylbenzoate hydrochloride (0.43 g, 2.11 mmol), triethylamine (0.48 g, 4.80 mmol) and tetrahydrofuran (10 ml). This was stirred at room temperature for 30 minutes, water was added, and tetrahydrofuran was distilled off. The residue was diluted ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (1:1)] to obtain methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylbenzoate (0.99 g, 1.48 mmol, 77%) as a colorless amorphous powder.

[α]_{D}²²−134.5° (c=0.18, methanol).

IR νₘₐₓ (KBr) cm⁻¹: 3317 (NH), 1722, 1682 (C═O).

¹H-NMR (CDCl₃) δ: 0.963 (3H, s), 1.022 (3H, s), 2.024 (3H, s), 2.269 (3H, s), 2.851 (1H, dd, J=4.4, 13.6 Hz), 3.076 (1H, dd, J=8.0, 13.6 Hz), 3.543 (1H, d, J=14.4 Hz), 3.617 (3H, s), 3.724 (1H, d, J=11.0 Hz), 3.880 (1H, d, J=11.0 Hz), 3.885 (3H, s), 3.894 (3H, s), 4.419 (1H, dd, J=4.4, 8.0 Hz), 4.566 (1H, d, J=14.4 Hz), 6.302 (1H, s), 6.655 (1H, d, J=1.8 Hz), 6.96-7.38 (6H, m), 7.746 (1H, d, J=8.4 Hz), 7.795 (1H, s), 8.480 (1H, s).

Elemental Analysis (C₃₅H₃₉N₂O₉Cl) Cal'd: C, 63.01; H, 5.89; N, 4.20. Found: C, 63.05; H, 5.94; N, 4.05

(2) A mixture of methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylbenzoate (0.89 g, 1.33 mmol) obtained in Example 45-(1), a 1N aqueous sodium hydroxide solution (3 ml) and ethanol (10 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethyl acetate-hexane (1:1) to obtain 3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylbenzoic acid (0.62 g, 1.01 mmol, 76%) as colorless prisms.

mp.172-173° C.

[α]_{D}²²−148.2° (c=0.29, methanol).

IR νₘₐₓ (KBr) cm⁻: 3600-2400 (br, COOH, OH, NH), 1651 (C═O).

¹H-NMR (CDCl₃) δ: 0.661 (3H, s), 1.053 (3H, s), 2.300 (3H, s), 2.876 (1H, dd, J=5.6, 14.0 Hz), 3.103 (1H, dd, J=8.0, 14.0 Hz), 3.184 (1H, d, J=11.0 Hz), 3.401 (1H, d, J=14.2 Hz), 3.615 (3H, s), 3.636 (1H, d, J=11.0 Hz), 3.894 (3H, s), 4.44-4.52 (2H, m), 6.207 (1H, s), 6.632 (1H, s), 6.99-7.35 (6H, m), 7.703 (1H, s), 7.803 (1H, d, J=7.4 Hz), 8.464 (1H, s).

### EXAMPLE 46

3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylbenzoic acid

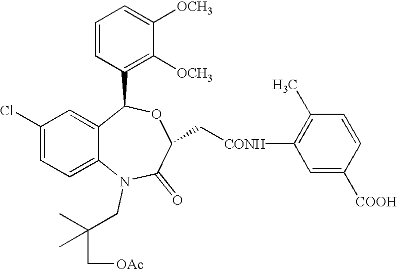

Acetyl chloride (0.13 g, 1.72 mmol) was added to a mixture of 3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylbenzoic acid (0.3 g, 0.491 mmol) obtained in Example 45-(2), pyridine (0.17 g, 2.21 mmol) and ethyl acetate (5 ml). After stirred at room temperature for 1 hour, water (4 ml) was added to the mixture, and the mixture was further stirred at room temperature for 3 hours. The organic layer was separated, and washed with 1N hydrochloric acid and an aqueous saturated solution of sodium chloride. This was dried with sodium sulfate, and concentrated under reduced pressure to obtain 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylbenzoic acid (0.28 g, 0.429 mmol, 87%) as a colorless amorphous powder.

[α]_{D}²²−132.7° (c=0.19, methanol)

IR νₘₐₓ (KBr) cm⁻¹: 3600-2400 (br, COOH, NH), 1724, 1678 (C═O).

¹H-NMR (CDCl₃) δ: 0.963 (3H, s), 1.022 (3H, s), 2.026 (3H, s), 2.288 (3H, s), 2.866 (1H, dd, J=4.6, 15.4 Hz), 3.096 (1H, dd, J=7.0, 15.4 Hz), 3.548 (1H, d, J=13.8 Hz), 3.617 (3H, s), 3.727 (1H, d, J=11.6 Hz), 3.884 (1H, d, J=11.6 Hz), 3.890 (3H, s), 4.438 (1H, dd, J=4.6, 7.0 Hz), 4.572 (1H, d, J=13.8 Hz), 6.304 (1H, s), 6.659 (1H, s), 6.97-7.33 (6H, m), 7.789 (1H, d, J=7.8 Hz), 7.868 (1H, s), 8.493 (1H, s).

Elemental analysis (C₃₄H₃₇N₂O₉Cl.H₂O) Cal'd: C, 60.85; H, 5.86; N, 4.17 Found: C, 60.94; H, 5.88; N, 3.92

### EXAMPLE 47

4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methylbenzoic acid

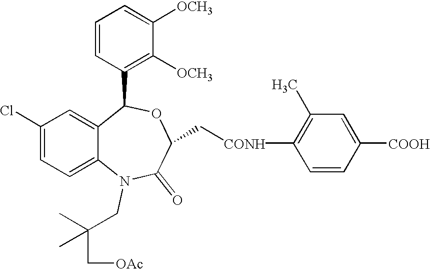

(1) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of benzyl 4-amino-3-methylbenzoate hydrochloride (0.59 g, 2.11 mmol), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (3:2)] to obtain benzyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-3-methylbenzoate (0.89 g, 1.20 mmol, 62%) as a colorless amorphous powder.

[α]_{D}²²−105.3° (c=0.12, methanol).

IR νₘₐₓ (KBr) cm⁻¹: 3360 (NH), 1714, 1682 (C═O).

¹H-NMR (CDCl₃) δ: 0.956 (3H, s), 1.015 (3H, s), 2.017 (3H, s), 2.244 (3H, s), 2.841 (1H, dd, J=5.6, 14.4 Hz), 3.089 (1H, dd, J=7.6, 14.4 Hz), 3.540 (1H, d, J=14.2 Hz), 3.616 (3H, s), 3.717 (1H, d, J=11.0 Hz), 3.882 (1H, d, J=11.0 Hz), 3.894 (3H, s), 4.380 (1H, dd, J=5.6, 7.6 Hz), 4.564 (1H, d, J=14.2 Hz), 5.343 (2H, s), 6.303 (1H, s), 6.658 (1H, d, J=1.8 Hz), 6.96-7.43 (11H, m), 7.88-8.21 (3H, m).

Elemental analysis (C₄₁H₄₃N₂O₉Cl) Cal'd: C, 66.26; H, 5.83; N, 3.77. Found: C, 66.04; H, 5.84; N, 3.79.

(2) 10% palladium carbon (0.1 g) was added to a solution of benzyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-3-methylbenzoate (0.8 g, 1.08 mmol) obtained in Example 47-(1) in ethyl acetate (20 ml), which was subjected to catalytic reduction at normal pressure for 3 hours. The catalyst was filtered to remove, and the solvent was distilled off to obtain 4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methylbenzoic acid (0.69 g, 1.06 mmol, 98%) as a colorless amorphous powder.

[α]_{D}²²−135.7° (c=0.23, methanol).

IR νₘₐₓ (KBr) cm⁻¹: 3400-2400 (br, COOH, NH), 1730, 1682 (C═O).

¹H-NMR (CDCl₃) δ: 0.963 (3H, s), 1.018 (3H, s), 2.020 (3H, s), 2.264 (3H, s), 2.861 (1H, dd, J=4.4, 14.0 Hz), 3.112 (1H, dd, J=7.6, 14.0 Hz), 3.547 (1H, d, J=14.4 Hz), 3.618 (3H, s), 3.721 (1H, d, J=11.2 Hz), 3.887 (1H, d, J=11.2 Hz), 3.896 (3H, s), 4.391 (1H, dd, J=4.4, 7.6 Hz), 4.570 (1H, d, J=14.4 Hz), 6.306 (1H, s), 6.659 (1H, d, J=2.0 Hz), 6.96-7.35 (6H, m), 7.80-8.25 (3H, m).

Elemental analysis (C₃₄H₃₇N₂O₉Cl) Cal'd: C, 62.53; H, 5.71; N, 4.29 Found: C, 63.27; H, 5.75; N, 4.04.

### EXAMPLE 48

4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-3-methylbenzoic acid

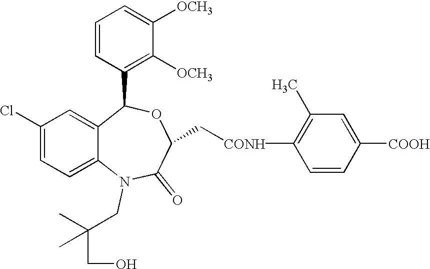

A mixture of 4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methylbenzoic acid (0.3 g, 0.459 mmol) obtained in Example 47-(2), a 1N aqueous sodium hydroxide solution (1 ml) and ethanol (3 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethanol-hexane (1:3) to obtain 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-3-methylbenzoic acid (0.17 g, 0.278 mmol, 61%) as colorless prisms.

mp.275-276° C.

[α]_{D}²²−143.1° (c=0.16, methanol).

IR νₘₐₓ (KBr) cm⁻¹: 3600-2400 (br, COOH, OH), 1685, 1635 (C═O).

¹H-NMR (CDCl₃) δ: 0.676 (3H, s), 1.042 (3H, s), 2.267 (3H, s), 2.898 (1H, dd, J=5.2, 13.6 Hz), 3.099 (1H, dd, J=6.8, 13.6 Hz), 3.152 (1H, d, J=13.2 Hz), 3.422 (1H, d, J=14.6 Hz), 3.599 (1H, d, J=13.2 Hz), 3.606 (3H, s), 3.898 (3H, s), 4.42-4.51 (2H, m), 6.203 (1H, s), 6.621 (1H, s), 6.97-7.37 (6H, m), 7.87-8.24 (3H, m).

Elemental analysis (C₃₂H₃₅N₂O₈Cl) Cal'd: C, 62.90; H, 5.77; N, 4.58. Found: C, 62.88; H, 5.66; N, 4.45.

### EXAMPLE 49

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-ethoxyphenyl]propionic Acid

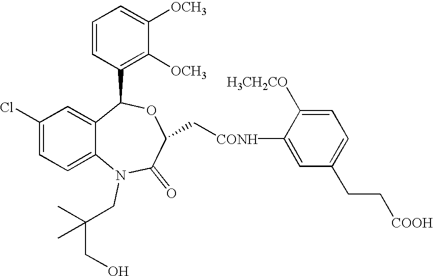

(1) A mixture of 4-hydroxy-3-nitrobenzaldehyde (2 g, 12.0 mmol), potassium carbonate (2.5 g, 18.0 mmol), iodoethane (2.4 g, 15.6 mmol) and N,N-dimethylformamide (20 ml) was stirred at 50° C. for 5 hours. This mixture was diluted with water, and extracted with ethyl acetate (100 ml). The extract was washed with saturated brine, dried with sodium sulfate, and concentrated under reduced pressure to obtain 4-ethoxy-3-nitrobenzaldehyde (2.48 g, 12.7 mmol, 100%) as a yellow oil.

IR νₘₐₓ (KBr) cm⁻¹: 1699 (C═O).

¹H-NMR (CDCl₃) δ: 1.528 (3H, t, J=7.4 Hz), 4.302 (2H, q, J=7.4 Hz), 7.213 (1H, d, J=8.8 Hz), 8.066 (1H, dd, J=2.2, 8.8 Hz), 8.330 (1H, d, J=2.2 Hz), 9.932 (1H, s).

(2) A mixture of 4-ethoxy-3-nitrobenzaldehyde (2.48 g, 12.7 mmol) obtained in Example 49-(1), (carboethoxymethylene)triphenylphosphine (4.8 g, 13.7 mmol) and tetrahydrofuran (30 ml) was stirred at 0° C. for 30 minutes. After stirred at room temperature for 3 hours, this mixture was diluted with ethyl acetate (100 ml), and washed with 1N hydrochloric acid (15 ml), an aqueous saturated sodium bicarbonate solution and saturated brine. The mixture was dried with sodium sulfate, and concentrated under reduced pressure. The residue was purified by silica gel column chromatography [hexane-ethyl acetate (2:1)] and recrystallization from ethyl acetate-hexane (1:5) to obtain ethyl 3-(4-ethoxy-3-nitrophenyl)-2-propenoate (3.18 g, 12.0 mmol, 94%) as yellow prisms.

mp.90-92° C.

IR νₘₐₓ (KBr) cm⁻: 1709 (C═O), 1637 (C═C).

¹H-NMR (CDCl₃) δ: 1.337 (3H, t, J=7.0 Hz), 1.491 (3H, t, J=7.0 Hz), 4.17-4.32 (4H, m), 6.379 (1H, d, J=16.0 Hz), 7.082 (1H, d, J=8.8 Hz), 7.603 (1H, d, J=16.0 Hz), 7.657 (1H, dd, J=2.2, 8.8 Hz), 7.988 (1H, d, J=2.2 Hz).

Elemental analysis (C₁₃H₁₅NO₅) Cal'd: C, 58.86; H, 5.70; N, 5.28. Found: C, 58.90; H, 5.74; N, 5.18.

(3) 10% palladium carbon (0.3 g) was added to a solution of ethyl 3-(4-ethoxy-3-nitrophenyl)-2-propenoate (2.9 g, 10.9 mmol) obtained in Example 49-(2) in ethanol (60 ml), which was subjected to normal pressure catalytic reduction at room temperature for 5 hours. The catalyst was filtered to remove, and the filtrate was concentrated under reduced pressure. The residue was dissolved in ethyl acetate (50 ml), and a 4N solution of hydrogen chloride in ethyl acetate (3 ml). The solvent was distilled off, and the residue was washed with ethyl acetate-hexane (1:1) to obtain ethyl 3-(3-amino-4-ethoxyphenyl)propionate hydrochloride (2.5 g, 9.13 mmol, 84% as colorless needles

mp.158-161° C.

IR νₘₐₓ (KBr) cm⁻¹: 3100-2400 (br, NH⁺), 1724 (C═O).

¹H-NMR (D₂O) δ: 0.783 (3H, t, J=7.0 Hz), 1.025 (3H, t, J=7.0 Hz), 2.323 (2H, t, J=6.2 Hz), 2.550 (2H, t, J=6.2 Hz), 3.719 (2H, q, J=7.0 Hz), 3.813 (2H, q, J=7.0 Hz), 6.749 (1H, d, J=8.4 Hz), 6.870 (1H, d, J=2.2 Hz), 6.936 (1H, dd, J=2.2, 8.4 Hz).

Elemental analysis (C₁₃H₂₀NO₃Cl) Cal'd: C, 57.04; H, 7.36; N, 5.12. Found: C, 56.97; H, 7.27; N, 5.10.

(4) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of ethyl 3-(3-amino-4-ethoxyphenyl)propionate hydrochloride (0.58 g, 2.11 mmol) obtained in Example 49-(3), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid and saturated brine, dried with sodium sulfate, and concentrated under reduced pressure. The residue was purified by silica gel column chromatography [eluent:ethyl acetate-hexane (1:1)] to obtain ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-ethoxyphenyl]propionate (0.7 g, 0.947 mmol, 49%) as a colorless amorphous powder.

[α]_{D}²²−143.8° (c=0.26, methanol).

IR νₘₐₓ (KBr) cm⁻¹: 3600-3200 (NH), 1732, 1682 (C═O).

¹H-NMR (CDCl₃) δ: 0.952 (3H, s), 1.024 (3H, s), 1.227 (3H, t, J=7.4 Hz), 1.368 (3H, t, J=7.4 Hz), 2.024 (3H, s), 2.570 (2H, t, J=7.8 Hz), 2.80-2.91 (3H, m), 3.044 (1H, dd, J=7.4, 15.0 Hz), 3.544 (1H, d, J=14.0 Hz), 3.606 (3H, s), 3.728 (1H, d, J=11.0 Hz), 3.865 (1H, d, J=11.0 Hz), 3.885 (3H, s), 4.00-4.16 (4H, m), 4.458 (1H, t, J=7.4 Hz), 4.577 (1H, d, J=14.0 Hz), 6.286 (1H, s), 6.629 (1H, d, J=2.0 Hz), 6.72-7.33 (7H, m), 8.15-8.21 (2H, m).

Elemental analysis (C₃₉H₄₇N₂O₁₀Cl) Cal'd: C, 63.36; H, 6.41; N, 3.79. Found: C, 63.00; H, 6.59; N, 3.67.

(5) A mixture of ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-ethoxyphenyl]propionate (0.6 g, 0.812 mmol) obtained in Example 49-(4), a 1N aqueous sodium hydroxide solution (2 ml) and ethanol (6 ml) was stirred at 60° C. for 30 minutes. This was diluted with water (50 ml) and, after acidification, extracted with ethyl acetate (100 ml). This was washed with saturated brine, dried with sodium sulfate, and concentrated under reduced pressure. The residue was purified by recrystallization from ethyl acetate:hexane (1:1) to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-ethoxyphenyl]propionic acid (0.51 g, 0.762 mmol, 94%) as colorless prisms.

mp151-153° C.

[α]_{D}²²−145.8° (c=0.27, methanol).

IR νₘₐₓ (KBr) cm⁻: 3600-2400 (br, COOH, NH, OH), 1730, 1714, 1658 (C═O).

¹H-NMR (CDCl₃) δ: 0.650 (3H, s), 1.055 (3H, s), 1.388 (3H, t, J=7.0 Hz), 2.624 (2H, t, J=6.8 Hz), 2.80-2.90 (3H, m), 3.097 (1H, dd, J=7.4, 14.6 Hz), 3.164 (1H, d, J=12.0 Hz), 3.392 (1H, d, J=14.6 Hz), 3.610 (3H, s), 3.644 (1H, d, J=12.0 Hz), 3.890 (3H, s), 4.040 (2H, q, J=7.0 Hz), 4.459 (1H, dd, J=5.4, 7.4 Hz), 4.489 (1H, d, J=14.6 Hz), 6.185 (1H, s), 6.613 (1H, s), 6.74-7.36 (7H, m), 8.18-8.20 (2H, m).

Elemental analysis (C₃₅H₄₁N₂O₉Cl.C₄H₈O₂) Cal'd: C, 61.86; H, 6.52; N, 3.70. Found: C, 61.81; H, 6.43; N, 3.70.

### EXAMPLE 50

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-ethoxyphenyl]propionic Acid

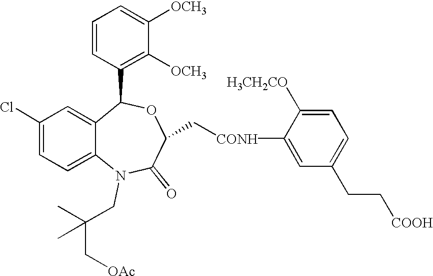

Acetyl chloride (86 mg, 1.10 mmol) was added to a mixture of 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-ethoxyphenyl]propionic acid (0.21 g, 0.314 mmol) obtained in Example 49-(5), pyridine (0.11 g, 1.41 mmol) and ethyl acetate (5 ml). After stirred at room temperature for 1 hour, water (4 ml) was added to this mixture, and the mixture was further stirred at room temperature for 2 hours. The organic layer was separated, and washed with 1N hydrochloric acid and saturated brine. This was dried with sodium sulfate, and concentrated under reduced pressure. The residue was recrystallized from ethyl acetate-hexane (1:2) to obtain 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-ethoxyphenyl]propionic acid (175 mg, 0.246 mmol, 78%) as colorless needles.

mp.175-176° C.

[α]_{D}²²−158.3° (c=0.31, methanol).

IR νₘₐₓ (KBr) cm⁻: 3400-2400 (br, COOH, NH), 1734, 1682 (C═O).

¹H-NMR (CDCl₃) δ: 0.954 (3H, s), 1.024 (3H, s), 1.368 (3H, t, J=7.0 Hz), 2.027 (3H, s), 2.628 (2H, t, J=8.0 Hz), 2.81-2.91 (3H, m), 3.051 (1H, dd, J=7.0, 14.4 Hz), 3.548 (1H, d, J=13.8 Hz), 3.606 (3H, s), 3.730 (1H, d, J=11.4 Hz), 3.870 (1H, d, J=11.4 Hz), 3.885 (3H, s), 4.025 (2H, q, J=7.0 Hz), 4.458 (1H, t, J=7.0 Hz), 4.580 (1H, d, J=13.8 Hz), 6.290 (1H, s), 6.630 (1H, s), 6.73-7.33 (7H, m), 8.17-8.22 (2H, m).

Elemental analysis (C₃₇H₄₃N₂O₁₀Cl) Cal'd: C, 62.49; H, 6.09; N, 3.94. Found: C, 62.31; H, 5.93; N, 3.80.

### EXAMPLE 51

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-isopropoxyphenyl]propionic Acid

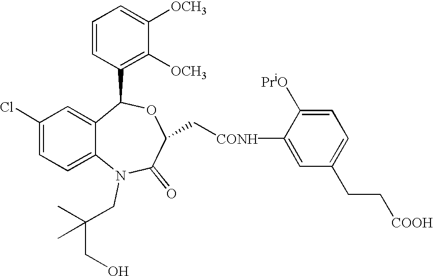

(1) A mixture of 4-hydroxy-3-nitrobenzaldehyde (2 g, 12.0 mmol), potassium carbonate (2.5 g, 18.0 mmol), 2-bromopropane (2.3 g, 18.0 mmol), sodium iodide (3.0 g, 20.0 mmol) and N,N-dimethylformamide (20 ml) was stirred at 50° C. overnight. This mixture was diluted with water, and extracted with ethyl acetate (100 ml). The extract was washed with saturated brine, dried with sodium sulfate, and concentrated under reduced pressure to obtain 4-isopropoxy-3-nitrobenzaldehyde (1.2 g, 5.74 mmol, 48%) as a yellow oil.

IR νₘₐₓ (KBr) cm⁻¹: 1699 (C═O).

¹H-NMR (CDCl₃) δ: 1.456 (6H, d, J=6.2 Hz), 4.73-4.92 (1H, m), 7.207 (1H, d, J=8.8 Hz), 8.045 (1H, dd, J=2.2, 8.8 Hz), 8.292 (1H, d, J=2.2 Hz), 9.918 (1H, s)

(2) A mixture of 4-isopropoxy-3-nitrobenzaldehyde (1.2 g, 5.74 mmol) obtained in Example 51-(1), (carboethoxymethylene)triphenylphosphine (2.2 g, 6.19 mmol) and tetrahydrofuran (20 ml) was stirred at 0° C. for 30 minutes. After further stirred at room temperature for 3 hours, this mixture was diluted with ethyl acetate (100 ml), and washed with 1N hydrochloric acid (10 ml), an aqueous saturated sodium bicarbonate solution and saturated brine. The mixture was dried with sodium sulfate, and concentrated under reduced pressure. The residue was purified by silica gel column chromatography [hexane-ethyl acetate (3:1)] to obtain ethyl 3-(4-isopropoxy-3-nitrophenyl)-2-propenoate (1.63 g, 5.84 mmol, 100%) as a yellow oil.

IR νₘₐₓ (KBr) cm⁻¹: 1712 (C═O), 1639 (C═C).

¹H-NMR (CDCl₃) δ: 1.339 (3H, t, J=7.0 Hz), 1.419 (6H, d, J=6.2 Hz), 4.269 (2H, q, J=7.0 Hz), 4.64-4.82 (1H, m), 6.373 (1H, d, J=15.6 Hz), 7.087 (1H, d, J=9.2 Hz), 7.603 (1H, d, J=15.6 Hz), 7.642 (1H, dd, J=2.2, 9.2 Hz), 7.949 (1H, d, J=2.2 Hz).

Elemental analysis (C₁₄H₁₇NO₅) Cal'd: C, 60.21; H, 6.14; N, 5.02. Found: C, 59,89; H, 6,05; N, 4.98.

(3) 10% palladium carbon (0.2 g) was added to a solution of ethyl 3-(4-isopropoxy-3-nitrophenyl)-2-propenoate (1.4 g, 5.12 mmol) obtained in Example 51-(2) in ethanol (40 ml). The mixture was subjected to normal pressure catalytic reduction at room temperature for 5 hours, the catalyst was filtered to remove, and the filtrate was concentrated under reduced pressure. The residue was dissolved in ethyl acetate (50 ml), and a 4N solution of hydrogen chloride in ethyl acetate (3 ml) was added thereto. The solvent was distilled off, and the residue was purified by recrystallization from ethyl acetate-hexane (1:1) to obtain ethyl 3-(3-amino-4-isopropxyphenyl)propionate hydrochloride (1.1 g, 3.82 mmol, 75%) as colorless prisms.

mp.115-122° C.

IR νₘₐₓ (KBr) cm⁻¹: 3100-2400 (br, NH⁺), 1724 (C═O).

¹H-NMR (CDCl₃) δ: 0.993 (3H, t, J=7.0 Hz), 1.179 (6H, d, J=6.2 Hz), 2.529 (2H, t, J=7.2 Hz), 2.756 (2H, t, J=7.2 Hz), 3.929 (2H, q, J=7.0 Hz), 4.52-4.61 (1H, m), 6.987 (1H, d, J=8.8 Hz), 7.080 (1H, d, J=1.8 Hz), 7.133 (1H, dd, J=1.8, 8.8 Hz).

(4) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1) and N,N-dimethylformamide (0.03 ml) in tetrahydrofuran (10 ml) at room temperature. After stirred for 1 hour, the mixture was concentrated under reduced pressure. The residue was dissolved in tetrahydrofuran (5 ml), which was added to a mixture of ethyl 3-(3-amino-4-isopropxyphenyl)propionate hydrochloride (0.61 g, 2.11 mmol) obtained in Example 51-(3), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid and saturated brine, dried with sodium sulfate, and concentrated under reduced pressure. The residue was purified by silica gel column chromatography [eluent:ethyl acetate-hexane (3:2)] to obtain ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-isopropoxyphenyl]propionate (0.76 g, 1.01 mmol, 53%) as a colorless amorphous powder.

[α]_{D}²²−131.6° (c=0.50, methanol).

IR νₘₐₓ (KBr) cm⁻¹: 3500-3200 (NH), 1732, 1682 (C═O).

¹H-NMR (CDCl₃) δ: 0.952 (3H, s), 1.024 (3H, s), 1.222 (3H, t, J=7.0 Hz), 1.305 (3H, d, J=6.4 Hz), 1.346 (3H, d, J=6.4 Hz), 2.026 (3H, s), 2.570 (2H, t, J=7.4 Hz), 2.78-2.90 (3H, m), 3.074 (1H, dd, J=7.2, 15.0 Hz), 3.543 (1H, d, J=14.6 Hz), 3.599 (3H, s), 3.732 (1H, d, J=11.0 Hz), 3.867 (1H, d, J=11.0 Hz), 3.879 (3H, s), 4.109 (2H, q, J=7.4 Hz), 4.43-4.61 (3H, m), 6.2796 (1H, s), 6.632 (1H, s), 6.74-7.33 (7H, m), 8.15-8.21 (2H, m).

Elemental analysis (C₄₀H₄₉N₂O₁₀Cl.0.5H₂O) Cal'd: C, 63.03; H, 6.61; N, 3.67. Found: C, 63,11; H, 6,63; N, 3.56.

(5) A mixture of ethyl 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-isopropoxyphenyl]propionate (0.66 g, 0.876 mmol) obtained in Example 51-(4), 1N aqueous sodium hydroxide solution (2 ml) and ethanol (6 ml) was stirred at 60° C. for 30 minutes. This was diluted with water (50 ml) and, after acidification, extracted with ethyl acetate (100 ml). This was washed with saturated brine, dried with sodium sulfate, and concentrated under reduced pressure. The residue was purified by recrystallization form ethyl acetate-hexane (1:1) to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-isopropoxyphenyl]propionic acid (0.51 g, 0.74 mmol, 85%) as colorless prisms.

mp.133-136° C.

[α]_{D}²²−118.5° (c=0.21, methanol).

IR νₘₐₓ (KBr) cm⁻: 3600-2400 (br, COOH, NH, OH), 1716, 1660 (C═O).

¹H-NMR (CDCl₃) δ: 0.646 (3H, s), 1.053 (3H, s), 1.325 (3H, d, J=6.2 Hz), 1.357 (3H, d, J=6.2 Hz), 2.628 (2H, t, J=8.0 Hz), 2.780-2.92 (3H, m), 3.132 (1H, dd, J=7.2, 14.0 Hz), 3.167 (1H, d, J=11.8 Hz), 3.388 (1H, d, J=14.2 Hz), 3.608 (3H, s), 3.650 (1H, d, J=11.8 Hz), 3.888 (3H, s), 4.45-4.59 (3H, m), 6.178 (1H, s), 6.625 (1H, s), 6.76-7.36 (7H, m), 8.18-8.20 (2H, m).

Elemental analysis (C₃₆H₄₃N₂O₉Cl.H₂O) Cal'd: C, 61.66; H, 6.47; N, 4.00. Found: C, 61,93; H, 6,52; N, 3.63.

### EXAMPLE 52

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-isopropoxyphenyl]propionic Acid

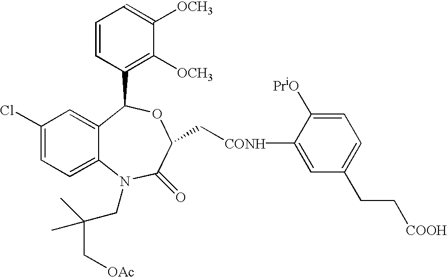

Acetyl chloride (80 mg, 1.02 mmol) was added to a mixture of 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-isopropoxyphenyl]propionic acid (0.20 g, 0.293 mmol) obtained in Example 51-(5), pyridine (0.10 g, 1.32 mmol) and ethyl acetate (5 ml). After stirred at room temperature for 1 hour, water (4 ml) was added to this mixture, and the mixture was further stirred at room temperature for 2 hours. The organic layer was separated, and washed with 1N hydrochloric acid and saturated brine. This was dried with sodium sulfate, and concentrated under reduced pressure. The residue was purified by recrystallization form ethyl acetate-hexane (1:2) to obtain 3-[3-[[[(3R,5S)-1-(3-acetoxy-2,2-dimethylpropyl)-7-chloro-5-(2,3-dimethoxyphenyl)-2-oxo-1,2,3,5-tetrahydro-4.1-benzoxazepin-3-yl]acetyl]amino]-4-isopropoxyphenyl]propionic acid (155 mg, 0.214 mmol, 73%) as colorless needles.

mp.101-103° C.

[α]_{D}²²−122.3° (c=0.19, methanol).

IR νₘₐₓ (KBr) cm⁻: 3400-2400 (br, COOH, NH), 1732, 1678 (C═O).

¹H-NMR (CDCl₃) δ: 0.956 (3H, s), 1.027 (3H, s), 1.310 (3H, d, J=5.8 Hz), 1.352 (3H, d, J=5.8 Hz), 2.031 (3H, s), 2.630 (2H, t, J=7.8 Hz), 2.79-2.91 (3H, m), 3.084 (1H, dd, J=7.2, 14.6 Hz), 3.549 (1H, d, J=14.4 Hz), 3.605 (3H, s), 3.733 (1H, d, J=11.0 Hz), 3.871 (1H, d, J=11.0 Hz), 3.885 (3H, s), 4.43-4.62 (3H, m), 6.283 (1H, s), 6.634 (1H, s), 6.75-7.33 (7H, m), 8.17-8.22 (2H, m).

Elemental analysis (C₃₈H₄₅N₂O₁₀Cl) Cal'd: C, 62.93; H, 6.25; N, 3.86. Found: C, 63,32; H, 6,56; N, 3.63.

### EXAMPLE 53

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic Acid

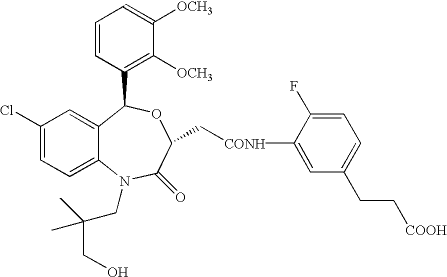

(1) Iodomethane (3.0 g) and potassium carbonate (2.7 g) were added to a solution of 4-fluoro-3-nitrobenzoic acid (3.0 g) in N,N-dimethylformamide (30 ml), and the mixture was stirred at room temperature for 30 minutes. The reaction solution was diluted by the addition of ethyl acetate (100 ml), washed with 1N hydrochloric acid, dried with anhydrous sodium sulfate, and concentrated under the reduced pressure. The residue was dissolved in methanol (100 ml), and 10% palladium carbon (0.5 g) was added to stir for 4 hours under hydrogen gas atmosphere. The reaction solution was filtered, and the filtrate was concentrated under the reduced pressure. A solution of the residue in tetrahydrofuran (10 ml) was added dropwise to a suspension of aluminum lithium hydride (1.2 g) in tetrahydrofuran (30 ml) for 10 minutes while stirring at room temperature. The reaction solution was heated to reflux for 1 hour, ice-cooled, and degraded with water (1.2 ml) and 1N sodium hydroxide (3.6 ml). The insolubles were filtered, and the filtrate was concentrated under the reduced pressure. Anhydrous trifluoroacetic acid (3.3 g) was added to a solution of the residue in ethyl acetate (40 ml), and the mixture was stirred at room temperature for 30 minutes. An aqueous sodium bicarbonate solution was added to the reaction solution, the organic layer was separated, and dried with anhydrous sodium sulfate. The solvent was concentrated under reduced pressure, and the residue was purified by silica gel column chromatography (eluent, hexane:ethyl acetate=3:1) to obtain 4-fluoro-3-trifluoroacetylaminobenzyl alcohol (2.5 g) as colorless crystals.

¹H-NMR (CDCl₃) δ: 4.690 (2H, s), 7.12-7.35 (2H, m), 8.05-8.35 (2H, m).

(2) Manganese dioxide (4.0 g) was added to a solution of 4-fluoro-3-trifluoroacetylaminobenzyl alcohol (2.5 g) obtained in Example 53-(1) in tetrahydrofuran (40 ml), and the mixture was stirred at room temperature for 20 hours. The reaction solution was filtered, and concentrated under the reduced pressure. The residue was purified by silica gel column chromatography (eluent, hexane:ethyl acetate=4:1) to obtain 4-fluoro-3-trifluoroacetylaminobezaldehyde (1.6 g) as colorless crystals.

¹H-NMR (CDCl₃) δ: 7.23-7.42 (1H, m), 7.75-7.86 (1H, m), 8.05-8.35 (1H, m), 8.818 (1H, dd, J=2.0, 7.2 Hz), 9.988 (1H, s).

(3) Sodium hydride (0.28 g, 60%) was added to a solution of 4-fluoro-3-trifluoroacetylaminobezaldehyde (1.4 g) obtained in Example 53-(2) and diethylphosphonoacetic acid ethyl ester (1.6 g) in tetrahydrofuran (40 ml), and the mixture was stirred at 60° C. for 2 hours. The reaction solution was diluted with ethyl acetate (30 ml), washed with a 5% aqueous potassium hydrogen sulfate solution, and an aqueous saturated sodium bicarbonate solution and water, dried with anhydrous sodium sulfate, and concentrated under the reduced pressure. The residue was purified by silica gel column chromatography (eluent, hexane:ethyl acetate=20:1) to obtain ethyl 4-fluoro-3-trifluoroacetylaminocinnamate (1.3 g) as colorless crystals.

¹H-NMR (CDCl₃) δ: 1.324 (3H, t, J=7.2 Hz), 4.271 (2H, q, J=7.2 Hz), 6.424 (1H, d, J=15.8 Hz), 7.14-7.45 (2H, m), 7.634 (1H, d, J=15.8 Hz), 7.95-8.25 (1H, dd, J=2.2, 7.5 Hz).

(4) 10% palladium carbon (0.2 g) was added to a solution of ethyl 4-fluoro-3-trifluoroacetylaminocinnamate (1.2 g) obtained in Example 53-(3) in ethanol (20 ml), and the mixture was stirred for 90 minutes in hydrogen stream. The reaction solution was filtered, and the filtrate was concentrated under the reduced pressure. The residue was purified by silica gel chromatography (eluent, hexane:ethyl acetate=4:1) to obtain ethyl 3-(4-fluoro-3-trifluoroacetylaminophenyl)propionate (1.15 g) as a colorless oil.

¹H-NMR (CDCl₃) δ: 1.239 (3H, t, J=7.2 Hz), 2.615 (2H, t, J=7.2 Hz), 2.952 (2H, t, J=7.8 Hz), 4.130 (2H, q, J=7.2 Hz), 6.95-7.15 (2H, m), 7.95-8.25 (2H, m).

(5) Method A: Sodium borohydride (0.4 g) was added to a solution of ethyl 3-(4-fluoro-3-trifluoroacetylaminophenyl)propionate (1.15 g) obtained in Example 53-(4) in ethanol (20 ml), and the mixture was stirred at 60° C. for 1 hour. The reaction solution was concentrated, extracted with ethyl acetate, washed with water, and dried with anhydrous sodium sulfate. The solvent was concentrated under the reduced pressure, the residue was purified by silica gel chromatography (eluent, hexane:ethyl acetate=10:1), and 10% hydrochloric acid (methanol solution) was added to the resulting colorless oil (0.9 g) to convert it into hydrochloride, to obtain ethyl 3-(3-amino-4-fluorophenyl)propionate (0.83 g) as colorless crystals.

Method B: A 1M solution of borane-tetrahydrofuran (67 ml, 67 mmol) was added dropwise to a solution of 4-fluoro-3-nitrobenzoic acid (5.0 g, 27.0 mmol) in tetrahydrofuran (50 ml) under ice-cooling, and the mixture was stirred at 70° C. for 2 hours. Water (10 ml) was added to the reaction solution under ice-cooling to stop the reaction, and the solvent was distilled off. Water (100 ml) was added to the residue, the mixture was extracted with ethyl acetate (100 ml) 2 times. The extract was washed with 1N hydrochloric acid and an aqueous saturated sodium bicarbonate solution, dried with anhydrous magnesium sulfate, and the solvent was distilled off under the reduced pressure. The residue was purified by silica gel column chromatography [eluent:hexane-ethyl acetate=4:1, then 2:1] to obtain 4-fluoro-3-nitrobenzyl alcohol (4.5 g, 97%) as a colorless oil.

¹H-NMR (CDCl₃) δ: 2.05 (1H, t, J=5.6 Hz), 4.78 (2H, d, J=5.6 Hz), 7.30 (1H, dd, J=10.6, 8.8 Hz), 7.60-7.75 (1H, m), 8.09 (1H, dd, J=6.6, 2.2 Hz).

A suspension of pyridine-sulfur trioxide complex (4.65 g, 29.2 mmol) in dimethylsulfoxide (12 ml) was added to a solution of 4-fluoro-3-nitrobenzyl alcohol (1.0 g, 5.84 mmol) obtained above and triethylamine (4.07 ml, 29.2 mmol) in dichloromethane (20 ml). The mixture was stirred at room temperature for 15 minutes, the reaction solution was diluted with diethyl ether (150 ml), washed with water, 5% potassium hydrogen sulfate and water, dried with anhydrous magnesium sulfate, and the solvent was distilled off under the reduced pressure. The residue was purified by silica gel column chromatography [eluent:hexane-ethyl acetate (5:1)] to obtain 4-fluoro-3-nitrobenzaldehyde (0.86 g, 87%) as colorless crystals.

mp.37-38° C.

¹H-NMR (CDCl₃) δ: 7.51 (1H, t, J=9.4 Hz), 8.10-8.30 (1H, m), 8.60 (1H, dd, J=7.4, 2.2 Hz), 10.05 (1H, s).

A mixture of 4-fluoro-3-nitrobenzaldehyde (9.4 g, 66.8 mmol) obtained above, (carboethoxymethylene)triphenylphosphine (2.2 g, 21.4 mmol) and tetrahydrofuran (100 ml) was stirred at 0° C. for 30 minutes. After further stirred at room temperature for 3 hours, this mixture was diluted with ethyl acetate (100 ml), and washed with 1N hydrochloric acid (80 ml), an aqueous saturated sodium bicarbonate solution and saturated brine. The mixture was dried with sodium sulfate, and concentrated under the reduced pressure. The residue was purified with recrystallization from ethyl acetate-hexane (1:2) to obtain ethyl 3-(4-fluoro-3-nitrophenyl)-2-propenoate (10.0 g, 41.6 mmol, 62%) as yellow needles.

mp.115-117° C.

IR νₘₐₓ (KBr) cm⁻¹: 1709 (C═O), 1637 (C═C).

¹H-NMR (CDCl₃) δ: 1.337 (3H, t, J=7.0 Hz), 1.491 (3H, t, J=7.0 Hz), 4.17-4.32 (4H, m), 6.379 (1H, d, J=16.0 Hz), 7.082 (1H, d, J=8.8 Hz), 7.603 (1H, d, J=16.0 Hz), 7.657 (1H, dd, J=2.2, 8.8 Hz), 7.988 (1H, d, J=2.2 Hz).

Elemental analysis (C₁₁H₁₀NO₄F) Cal'd: C, 55.23; H, 4.21:N, 5.86 Found: C, 55.29; H, 4.15; N, 5.67

10% palladium carbon (0.5 g) was added to a solution of ethyl 3-(4-fluoro-3-nitrophenyl)-2-propenoate (5 g, 20.9 mmol) obtained above in ethanol (100 ml), the mixture was subjected to normal pressure catalytic reduction at room temperature for 4 hours. The catalyst was filtered to remove, and the filtrate was concentrated under reduced pressure. The residue was dissolved in ethyl acetate (50 ml), a 4N solution of hydrogen chloride in ethyl acetate (7 ml) was added. The solvent was distilled off, and the residue was washed with ethyl acetate-diethyl ether (1:1) to obtain ethyl 3-(4-amino-3-fluorophenyl)propionate hydrochloride (4.8 g, 19.4 mmol, 93%) as a colorless powder.

mp.105-115° C.

IR νₘₐₓ (KBr) cm⁻¹: 3200-2400 (br, NH₃⁺), 1730 (C═O).

¹H-NMR (D₂O) δ: 1.031 (3H, t, J=7.2 Hz), 2.579 (2H, t, J=6.6 Hz), 2.822 (2H, t, J=6.6 Hz), 3.960 (2H, q, J=7.2 Hz), 7.08-7.23 (3H, m).

Elemental analysis (C₁₁H₁₅NO₂ClF) Cal'd: C, 53.34; H, 6.10:N, 5.65 Found: C, 53.27; H, 5.93; N, 5.58

(6) Thionyl chloride (13.7 g) and N,N-dimethylformamide (0.2 ml) were added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (20 g) obtained in Example 1-(1) in tetrahydrofuran (200 ml), and the mixture was stirred at room temperature for 1 hour. The reaction solution was concentrated under reduced pressure, and the residue was dissolved in tetrahydrofuran (100 ml). This solution was added dropwise to a suspension of ethyl 3-(3-amino-4-fluorophenyl)propionate hydrochloride (10.5 g) obtained in Example 53-(5), triethylamine (10.7 g) and tetrahydrofuran (100 ml) for 30 minutes while stirring at room temperature. The reaction solution was stirred for 30 minutes, diluted with ethyl acetate (50 ml), washed successively with 5% potassium hydrogen sulfate, an aqueous saturated sodium bicarbonate and water, and dried with anhydrous sulfate. The solvent was concentrated under reduced pressure, and the residue was purified by silica gel column chromatography (eluent, hexane:ethyl acetate=2:1) to obtain ethyl 3-[3-[[(3R,5S)-1-(3-acetoxy-2,2-dimethylpropyl)-7-chloro-7-(2,3-dimethoxyphenyl)-2-oxo-1,2,3,5-tetrahydro-4,1-benzoxazepin-3-yl]acetyl]amino-4-fluorophenyl]propionate (24.3 g, 91%) as a colorless amorphous powder.

¹H-NMR (CDCl₃) δ: 0.956 (3H, s), 1.024 (3H, s), 1.227 (3H, t, J=7.0 Hz), 2.026 (3H, s), 2.580 (2H, t, J=7.8 Hz), 2.77-2.97 (3H, m), 3.060 (1H, dd, J=7.0, 16.3 Hz), 3.548 (1H, d, J=14.0 Hz), 3.621 (3H, s), 3.723 (1H, d, J=11.6 Hz), 3.868 (1H, d, J=11.6 Hz), 3.892 (3H, s), 4.115 (2H, q, J=7.0 Hz), 4.409 (1H, dd, J=5.6, 6.8 Hz), 4.584 (1H, d, J=14.0 Hz), 6.295 (1H, s), 6.653 (1H, d, J=1.6 Hz), 6.83-7.42 (7H, m), 7.95-8.05 (1H, m), 8.138 (1H, d, J=2.2 Hz).

(7) 1N sodium hydroxide (80 ml) was added to a solution of ethyl 3-[3-[[(3R,5S)-1-(3-acetoxy-2,2-dimethylpropyl)-7-chloro-7-(2,3-dimethoxyphenyl)-2-oxo-1,2,3,5-tetrahydro-4,1-benzoxazepin-3-yl]acetyl]amino-4-fluorophenyl]propionate (24.3 g) obtained in Example 53-(6) in ethanol (160 ml), and the mixture was stirred at 60° C. for 1.5 hours. The reaction solution was cooled, water (50 ml) was added, and the mixture was extracted with ether (30 ml). 1N hydrochloric acid was added to the aqueous layer to neutralize, which was extracted with ethyl acetate, washed with water, and dried with anhydrous sodium sulfate. The solvent was concentrated under reduced pressure, and the residue was recrystallized from ethanol-water (2:1) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-fluorophenyl]propionic acid (15.7 g, 70%) as colorless prisms.

mp.151-152° C.

¹H-NMR (CDCl₃) δ: 0.67 (3H, s), 1.07 (3H, s), 2.57-2.72 (2H, m), 2.78-3.25 (5H, m), 3.398 (1H, d, J=14.2 Hz), 3.615 (3H, s), 3.628 (1H, d, J=11.4 Hz), 4.38-4.55 (2H, m), 6.195 (1H, s), 6.638 (1H, d, J=1.8 Hz), 6.83-7.45 (7H, m), 7.92-8.15 (2H, m).

Elemental analysis (C₃₃H₃₆N₂O₈ClF) Cal'd: C, 61.63; H, 5.64; N, 4.36 Found: C, 61.72; H, 5.79; N, 4.13

### EXAMPLE 54

3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-fluorophenyl]propionic Acid

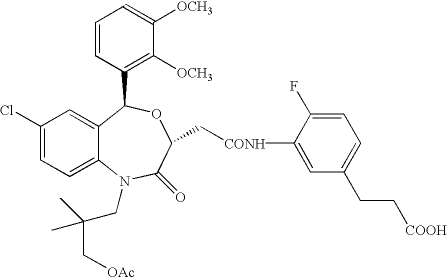

Acetyl chloride (0.13 g) and pyridine (0.16 g) were added to a solution of 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-fluorophenyl]propionic acid (0.3 g) obtained in Example 53-(7) in ethyl acetate (6 ml), and the mixture was stirred at room temperature for 1 hour. Water (8 ml) was added to the reaction solution, and the mixture was further stirred for 3 hours. The reaction solution was washed with 1N hydrochloric acid, washed with water, and dried with anhydrous sodium sulfate. The solvent was concentrated under the reduced pressure, and the residue was purified by silica gel chromatography (eluent, methylene chloride:methanol=20:1) to obtain 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-fluorophenyl]propionic acid (0.21 g) as a colorless amorphous powder.

¹H-NMR (CDCl₃) δ: 0.954 (3H, s), 1.020 (3H, s), 2.023 (3H, s), 2.638 (2H, t, J=7.4 Hz), 2.75-2.96 (3H, m), 3.066 (1H, dd, J=7.4, 14.7 Hz), 3.546 (1H, d, J=14.0 Hz), 3.619 (3H, s), 3.723 (1H, d, J=11.0 Hz), 3.867 (1H, d, J=11.0 Hz), 3.890 (3H, s), 4.408 (1H, dd, J=5.6, 7.3 Hz), 4.581 (1H, d, J=14.0 Hz), 6.294 (1H, s), 6.653 (1H, d, J=1.6 Hz), 6.83-7.45 (8H, m), 7.95-8.18 (2H, m).

### EXAMPLE 55

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methylphenyl]propionic Acid

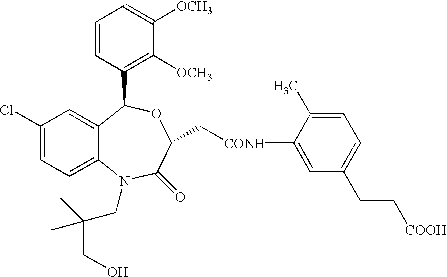

(1) A solution of 4-methyl-3-nitrobenzoic acid (2.0 g) and N-methylmorpholine (1.34 g) in tetrahydrofuran (30 ml) was cooled to −10° C., and ethyl chloroformate (1.44 g) and sodium borohydride (1.6 g) were added thereto. Then, methanol (16 ml) was added dropwise. The reaction solution was stirred at room temperature for 40 minutes, water (100 ml) was added, and extracted with ethyl acetate. The organic layer was washed with water, dried with anhydrous sodium sulfate, and concentrated under the reduced pressure. The residue was purified with silica gel chromatography (eluent, hexane:ethyl acetate=3:1, then 1:1), manganese dioxide (2.0 g) was added to a solution of the resulting oil (1.5 g) in tetrahydrofuran (30 ml), and the mixture was stirred at room temperature for 20 hours. The reaction solution was filtered, the filtrate was concentrated, and the residue was purified by silica gel chromatography (eluent, hexane:ethyl acetate=4:1) to obtain 4-methyl-3-nitrobenzaldehyde (0.5 g) as colorless crystals.

¹H-NMR (CDCl₃) δ: 2.708 (3H, s), 7.554 (1H, d, J=7.6 Hz), 8.031 (1H, dd, J=1.6, 7.6 Hz), 8.462 (1H, d, J=1.6 Hz), 10.046 (1H, s).

(2) Sodium hydride (0.15 g, 60%) was added to a solution of 4-methyl-3-nitrobenzaldehyde (0.5 g) obtained in Example 55-(1) and diethylphosphonoacetic acid ethyl ester (0.8 g) in tetrahydrofuran (15 ml), and the mixture was stirred at room temperature for 90 minutes. 1N hydrochloric acid was added to the reaction solution to degrade, which was extracted with ethyl acetate, washed with water, dried with anhydrous sodium sulfate, and concentrated under the reduced pressure. The residue was purified by silica gel chromatography (eluent, hexane:ethyl acetate=4:1) to obtain 4-methyl-3-nitrocinnamic acid ethyl ester (0.55 g) as colorless crystals.

¹H-NMR (CDCl₃) δ: 1.340 (3H, t, J=7.2 Hz), 2.629 (3H, s), 4.283 (2H, q, J=7.2 Hz), 6.495 (1H, d, J=16.0 Hz), 7.331 (1H, d, J=8.0 Hz), 7.58-7.73 (2H, m), 8.124 (1H, d, J=1.8 Hz).

(3) 10% palladium carbon (0.1 g) was added to a solution of 4-methyl-3-nitrocinnamic acid ethyl ester (0.5 g) obtained in Example 55-(2) in ethanol (15 ml), and the mixture was stirred in hydrogen stream for 3.5 hours. The reaction solution was filtered, and hydrochloric acid (4N solution in ethyl acetate) was added to the filtrate to obtain 3-(3-amino-4-methylphenyl)propionic acid ethyl ester hydrochloride (0.52 g) as crystals.

¹H-NMR (D₂O) δ: 1.231 (3H, t, J=7.4 Hz), 2.555 (3H, s), 2.599 (2H, t, J=8.0 Hz), 2.943 (2H, t, J=8.0 Hz), 4.108 (2H, q, J=7.4 Hz), 7.12-7.28 (2H, m), 7.436 (1H, s).

(4) Thionyl chloride (0.66 g) and N,N-dimethylformamide (0.1 ml) were added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (0.9 g) obtained in Example 1-(1) in tetrahydrofuran (20 ml), and the mixture was stirred at room temperature for 1 hour. The reaction solution was concentrated under the reduced pressure, toluene (20 ml) was added, and concentrated again. A solution of the residue in tetrahydrofuran (15 ml) was added dropwise to a solution of 3-(3-amino-4-methylphenyl)propionic acid ethyl ester hydrochloride (0.5 g) obtained in Example 55-(3), triethylamine (0.88 ml) and tetrahydrofuran (15 ml) for 5 minutes while stirring at room temperature. The reaction solution was stirred for 30 minutes, diluted with ethyl acetate (80 ml), washed with 1N hydrochloric acid and an aqueous saturated sodium bicarbonate solution, washed with water, dried with anhydrous sodium sulfate, and the solvent was distilled off. The residue was purified by silica gel chromatography (hexane:ethyl acetate=3:1-3:2) to obtain 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid ethyl ester (1.1 g) as a colorless amorphous powder.

¹H-NMR (CDCl₃) δ: 0.958 (3H, s), 1.022 (3H, s), 1.231 (3H, t, J=7.0 Hz), 2.204 (3H, s), 2.183 (3H, s), 2.584 (2H, t, J=7.6 Hz), 2.71-2.97 (3H, m), 3.073 (1H, dd, J=7.6, 14.0 Hz), 3.537 (1H, d, J=14.2 Hz), 3.614 (3H, s), 3.723 (1H, d, J=11.4 Hz), 3.868 (1H, d, J=11.4 Hz), 3.890 (3H, s), 4.117 (2H, q, J=7.0 Hz), 4.33-4.48 (1H, m), 4.563 (1H, d, J=14.2 Hz), 6.290 (1H, s), 6.648 (1H, d, J=2.0 Hz), 6.84-7.38 (7H, m), 7.65-7.78 (2H, m),

(5) 1N sodium hydroxide (6 ml) was added to a solution of 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid ethyl ester (1.0 g) obtained in Example 55-(4) in ethanol (10 ml), and the mixture was stirred at 60° C. for 40 minutes. Water (30 ml) was added to the reaction solution, extracted with ether, the aqueous layer was neutralized with 1N hydrochloric acid, and extracted with ethyl acetate ester. The organic layer was dried with anhydrous sodium sulfate, concentrated, and the residue was purified by silica gel chromatography (methylene chloride:methanol=15:1) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methylphenyl]propionic acid (0.78 g) as colorless crystals.

mp.154-155° C.

¹H-NMR (CDCl₃) δ: 0.655 (3H, s), 1.047 (1H, s), 2.170 (3H, s), 2.618 (2H, t, J=8.4 Hz), 2.75-3.12 (4H, m), 3.173 (1H, d, J=12.2 Hz), 3.392 (1H, d, J=14.4 Hz), 3.602 (3H, s), 3.623 (1H, d, J=12.2 Hz), 3.890 (3H, s), 4.37-4.55 (2H, m), 6.189 (1H, s), 6.637 (1H, d, J=1.6 Hz), 6.87-7.42 (6H, m), 7.55-7.68 (2H, m).

### EXAMPLE 56

3-[3-[[(3R,5S)-1-(3-acetoxy-2,2-dimthylpropyl)-7-chloro-5-(2,3-dimethoxyphenyl)-2-oxo-1,2,3,5-tetrahydro-4,1-benzoxazepin-3-yl]acetyl]amino-4-methylphenyl]propionic Acid

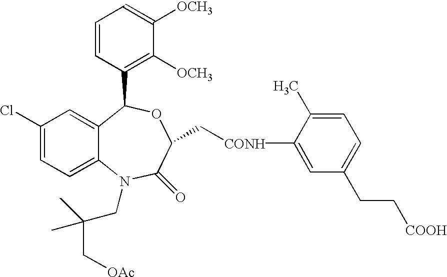

3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methylphenyl]propionic acid (0.5 g) obtained in Example 55-(5) was reacted and treated according to the synthesizing method of Example 54 to obtain 3-[3-[[(3R,5S)-1-(3-acetoxy-2,2-dimthylpropyl)-7-chloro-5-(2,3-dimethoxyphenyl)-2-oxo-1,2,3,5-tetrahydro-4,1-benzoxazepin-3-yl]acetyl]amino-4-methylphenyl]propionic acid (0.39 g) as a colorless amorphous powder.

¹H-NMR (CDCl₃) δ: 0.955 (3H, s), 1.017 (3H, s), 2.021 (3H, s), 2.171 (3H, s), 2.25-3.15 (6H, m), 3.536 (1H, d, J=13.8 Hz), 3.613 (3H, s), 3.713 (1H, d, J=11.0 Hz), 3.867 (1H, d, J=11.0 Hz), 3.889 (3H, s), 4.35-4.47 (1H, m), 4.556 (1H, d, J=13.8 Hz), 6.291 (1H, s), 6.651 (1H, d, J=1.2 Hz), 6.85-7.38 (6H, m), 7.750 (2H, d, J=9.8 Hz).

### EXAMPLE 57

3-[5-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-methoxylphenyl]propionic Acid

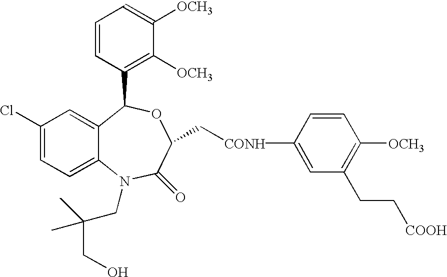

(1) Iodomethane (3.8 g) and sodium hydride (0.93 g) were added to a solution of 2-hydroxy-5-nitrobenzaldehyde (3.0 g) in N,N-dimethylformamide (20 ml), and the mixture was stirred at 60° C. for 1.5 hours. 1N hydrochloric acid was added to the reaction solution, extracted with ethyl acetate, washed with water, dried with anhydrous sodium sulfate, and concentrated. Diethylphosphonoacetic acid ethyl ester (4.2 g) and sodium hydride (0.82 g, 60%) were added to a solution of the residue (3.0 g) in tetrahydrofuran (50 ml), and the mixture was stirred at 60° C. for 30 minutes. The reaction solution was diluted by the addition of ethyl acetate (50 ml), washed with 5% potassium hydrogen sulfate, dried with anhydrous sodium sulfate, and concentrated. The residue was purified by silica gel chromatography (eluent, hexane:ethyl acetate=4:1) to obtain 2-methoxy-5-nitrocinnamic acid ethyl ester (2.0 g) as colorless crystals.

¹H-NMR (CDCl₃) δ: 1.353 (3H, t, J=7.2 Hz), 4.016 (3H, s), 4.288 (2H, q, J=7.2 Hz), 6.614 (1H, d, J=16.2 Hz), 7.002 (1H, d, J=9.0 Hz), 7.942 (1H, d, J=16.2 Hz), 8.257 (1H, dd, J=2.8, 9.0 Hz), 8.422 (1H, d, J=2.8 Hz).

(2) 10% palladium carbon (0.5 g) was added to a solution of 2-methoxy-5-nitrocinnamic acid ethyl ester (1.8 g) obtained in Example 57-(1) in ethanol (40 ml), and the mixture was stirred at room temperature for 1.5 hours in hydrogen stream. The reaction solution was filtered, and hydrogen chloride (ethyl acetate solution, 4N) was added thereto to obtain 3-(5-amino-2-methoxyphenyl)propionic acid ethyl ester hydrochloride (1.7 g, grayish white needles).

¹H-NMR (D₂O) δ: 1.234 (3H, t, J=7.2 Hz), 2.566 (2H, t, J=7.2 Hz), 2.85-3.02 (2H, m), 3.823 (3H, s), 4.120 (2H, q, J=7.2 Hz), 6.75-6.88 (1H, m), 7.15-7.45 (2H, m).

(3) (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g) obtained in Example 1-(1) and 3-(5-amino-2-methoxyphenyl)propionic acid ethyl ester hydrochloride (0.55 g) obtained in Example 57-(2) were reactioned and treated according to the synthesizing method of Example 55 to obtain 3-[5-[[[(3R,5S)-1-(3-acetoxy-2,2-dimethylpropyl)-7-chloro-5-5-(2,2-dimethoxyphenyl)-2-oxo-1,2,3,5-tetrahydro-4,1-benzoxazepin-3-yl]acetyl]amino]-2-mehtoxyphenyl]propionic acid ethyl ester (1.2 g) as a colorless amorphous powder.

¹H-NMR (CDCl₃) δ: 0.956 (3H, s), 1.022 (3H, s), 1.276 (3H, t, J=7.2 Hz), 2.025 (3H, s), 2.52-3.05 (6H, m), 3.533 (1H, d, J=14.0 Hz), 3.617 (3H, s), 3.729 (1H, d, J=11.4 Hz), 3.892 (3H, s), 4.122 (2H, q, J=7.2 Hz), 4.111 (1H, t, J=7.0 Hz), 4.559 (1H, d, J=9.0 Hz), 6.293 (1H, s), 6.636 (1H, d, J=2.0 Hz), 6.859 (1H, d, J=9.0 Hz), 6.95-7.42 (7H, m), 7.658 (1H, s).

(4) 3-[5-[[[(3R,5S)-1-(3-acetoxy-2,2-dimethylpropyl)-7-chloro-5-(2,2-dimethoxyphenyl)-2-oxo-1,2,3,5-tetrahydro-4,1-benzoxazepin-3-yl]acetyl]amino]-2-mehtoxyphenyl]propionic acid ethyl ester (1.2 g) obtained in Example 53-(3) was hydrolyzed using 1N sodium hydroxide (10 ml) to obtain 3-[5-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-methoxylphenyl]propionic acid (0.72 g) as a colorless amorphous powder.

¹H-NMR (CDCl₃) δ: 0.653 (3H, s), 1.046 (3H, s), 2.45-3.08 (6H, m), 3.184 (1H, d, J=11.8 Hz), 3.384 (1H, d, J=14.2 Hz), 3.610 (3H, s), 3.620 (1H, d, J=11.8 Hz), 3.795 (3H, s), 3.891 (3H, s), 4.38-4.55 (2H, m), 6.179 (1H, s), 6.621 (1H, d, J=1.8 Hz), 6.768 (1H, d, J=8.8 Hz), 6.93 -7.45 (7H, m), 7.819 (1H, s).

### EXAMPLE 58

3-[5-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-methoxylphenyl]propionic Acid

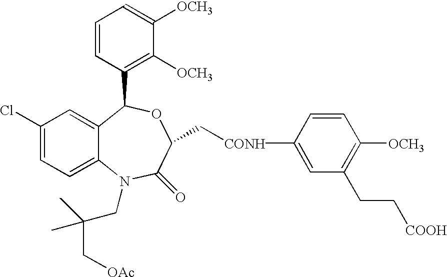

3-[5-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-methoxylphenyl]propionic acid (0.6 g) obtained in Example 57 (4) was reacted and treated according to the synthesizing method of Example 54 to obtain 3-[5-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-methoxylphenyl]propionic acid (0.4 g) as a colorless amorphous powder.

¹H-NMR (CDCl₃) δ: 0.947 (3H, s), 1.013 (3H, s), 2.010 (3H, s), 2.45-3.15 (6H, m), 3.532 (1H, d, J=14.2 Hz), 3.614 (3H, s), 3.733 (1H, d, J=11.2 Hz), 3.792 (3H, s), 3.864 (1H, d, J=11.2 Hz), 3.887 (3H, s), 4.431 (1H, dd, J=5.6, 7.6 Hz), 4.548 (1H, d, J=14.2 Hz), 6.287 (1H, s), 6.638 (1H, br), 6.757 (1H, d, J=9.0 Hz), 6.95-7.45 (7H, m), 7.957 (1H, s).

### EXAMPLE 59

3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenylacetic acid

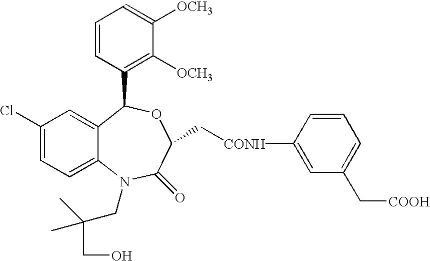

(1) (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g) obtained in Example 1-(1) was converted into acid chloride according to the method of Example 53, which was reacted with 3-aminophenylacetic acid methyl ester hydrochloride (0.43 g) to obtain 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetic acid methyl ester (0.85 g) as a colorless amorphous powder.

¹H-NMR (CDCl₃) δ: 0.959 (3H, s), 1.024 (3H, s), 2.025 (3H, s), 2.812 (1H, dd, J=5.6, 14.1 Hz), 3.002 (1H, dd, J=7.2, 14.1 Hz), 3.538 (1H, d, J=14.2 Hz), 3.608 (2H, s), 3.620 (3H, s), 3.690 (3H, s), 3.732 (1H, d, J=11.2 Hz), 3.870 (1H, d, J=11.2 Hz), 3.894 (3H, s), 4.403 (1H, dd, J=5.8, 7.2 Hz), 4.564 (1H, d, J=14.2 Hz), 6.299 (1H, s), 6.645 (1H, d, J=2.0 Hz), 6.95-7.48 (9H, m), 7.847 (1H, br).

(2) 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenylacetic acid methyl ester (0.8 g) obtained in Example 59-(1) was alkali-hydrolyzed according to the method of Example 53 to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenylacetic acid (0.27 g) as a colorless amorphous powder.

¹H-NMR (CDCl₃) δ: 0.645 (3H, s), 1.035 (3H, s), 2.809 (1H, dd, J=5.8, 14.2 Hz), 3.016 (1H, dd, J=7.8, 14.2 Hz), 3.173 (1H, d, J=11.8 Hz), 3.368 (1H, d, J=14.6 Hz), 3.604 (3H, s), 3.626 (1H, d, J=11.8 Hz), 3.887 (3H, s), 4.38-4.54 (2H, m), 6.177 (1H, s), 6.617 (1H, d, J=2.0 Hz), 6.93-7.48 (9H, m), 8.007 (1H, br).

### EXAMPLE 60

3-[3-[[(3R,5S)-7-chloro-5-(2,3-dimethoxyphenyl)-1-neopentyl-2-oxo-1,2,3,5-tetrahydro-4,1-benzoxazepin-3-yl]acetyl]aminophenyl]propionic Acid

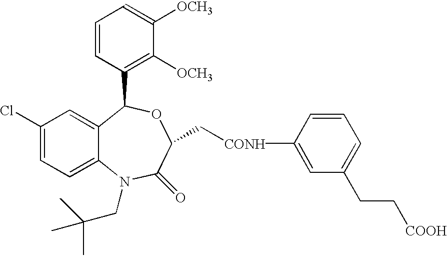

(1) (3R,5S)-7-chloro-5-(2,3-dimethoxyphenyl)-1-neopentyl-2-oxo-1,2,3,5-tetrahydro-4,1-benzoxazepine-3-acetic acid (1.0 g) was converted into acid chloride according to the method of Example 53, which was reacted with the compound (0.55 g) obtained in Example 35-(1) to obtain 3-[3-[[(3R,5S)-7-chloro-5-(2,3-dimethoxyphenyl)-2-oxo-1,2,3,5-tetrahydro-4,1-benzoxazepin-3-yl]acetyl]aminophenyl]propionic acid ethyl ester (1.1 g) as a colorless amorphous powder.

¹H-NMR (CDCl₃) δ: 0.954 (9H, s), 1.237 (3H, t, J=7.0 Hz), 2.601 (2H, t, J=7.2 Hz), 2.73-3.08 (4H, m), 3.361 (1H, d, J=14.0 Hz), 3.628 (3H, s), 3.894 (3H, s), 4.128 (2H, q, J=7.0 Hz), 4.408 (1H, dd, J=5.6, 7.3 Hz), 4.512 (1H, d, J=14.0 Hz), 6.308 (1H, s), 6.619 (1H, d, J=1.8 Hz), 6.88-7.43 (9H, m), 7.884 (1H, br).

(2) 1N sodium hydroxide (10 ml) was added to a solution of 3-[3-[[(3R,5S)-7-chloro-5-(2,3-dimethoxyphenyl)-2-oxo-1,2,3,5-tetrahydro-4,1-benzoxazepin-3-yl]acetyl]aminophenyl]propionic acid ethyl ester (1.0 g) obtained in Example 60-(1) in ethanol (5 ml), and the mixture was stirred at 60° C. for 1 hour. The reaction solution was diluted by the addition of water (30 ml), neutralized with 1N hydrochloric acid, and extracted with ethyl acetate. The organic layer was washed with water, dried with anhydrous sodium sulfate, and concentrated. The crystals obtained from the residue were recrystallized from ethyl acetate and hexane to obtain 3-[3-[[(3R,5S)-7-chloro-5-(2,3-dimethoxyphenyl)-1-neopentyl-2-oxo-1,2,3,5-tetrahydro-4,1-benzoxazepin-3-yl]acetyl]aminophenyl]propionic acid (0.9 g) as colorless crystals.

mp.172-173° C.

¹H-NMR (CDCl₃) δ: 0.943 (9H, s), 2.646 (2H, t, J=7.2 Hz), 2.73-3.13 (4H, m), 3.357 (1H, d, J=13.6 Hz), 3.625 (3H, s), 3.888 (3H, s), 4.428 (1H, dd, J=5.4, 6.9 Hz), 4.492 (1H, d, J=13.6 Hz), 6.299 (1H, s), 6.619 (1H, d, J=1.8 Hz), 6.88-7.42 (9H, m), 8.148 (1H, s).

### EXAMPLE 61

4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenylacetic acid

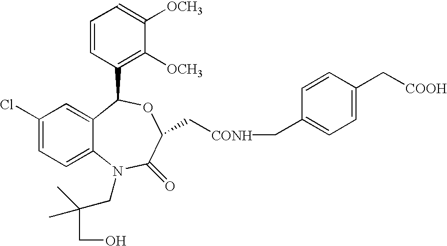

(1) Diphenylphosphorylazide (0.65 g, 2.38 mmol) was added to a mixture of 4-(methoxycarbonylmethyl)phenylacetic acid (5 g, 0.024 mol, triethylamine (3.0 g, 0.030 mol) and N,N-dimethylformamide (50 ml), and the mixture was stirred at room temperature for 30 minutes. The reaction solution was poured into water, and extracted with ethyl acetate (100 ml×2). The whole organic layer was washed with a 5% aqueous potassium hydrogen sulfate solution, an aqueous saturated potassium bicarbonate solution and saturated brine, dried with sodium sulfate, and the solvent was distilled off. The residue was dissolved in toluene (50 ml), which was heated at reflux for 1 hour, and concentrated under the reduced pressure. The residue was dissolved in tert-butanol (50 ml), and pyridine (3.8 g, 0.048 mol) was added. This mixture was heated at reflux for 5 hours. The mixture was diluted with ethyl acetate (100 ml), washed with 1N hydrochloric acid, an aqueous saturated sodium bicarbonate solution and saturated brine, dried with sodium sulfate, and concentrated under the reduced pressure. The residue was purified by silica gel column chromatography [eluent: hexane-ethyl acetate (3:1)]to obtain methyl 4-(tert-butoxycarbonylaminomethyl)phenylacetate (3.2 g, 11.5 mmol, 48%) as a colorless oil.

IR νₘₐₓ (KBr) cm⁻¹: 3358 (NH), 1738, 1712, 1699 (C═O).

¹H-NMR (CDCl₃) δ: 1.456 (9/10×9H, s), 1.641 (1/10×9H, s), 3.612 (2H, s), 3.687 (3H, s), 4.292 (9/10×2H, d, J=5.8 Hz), 4.394 (1/10×2H, d, J=6.0 Hz), 4.76-4.90 (1H, br), 7.242 (4H, m).

(2) A mixture of methyl 4-(tert-butoxycarbonylaminomethyl)phenylacetate (3.2 g, 11.5 mmol) obtained in Example 61-(1) and trifluoroacetic acid (15 mmol) was stirred at room temperature for 30 minutes, and the reaction solution was concentrated under the reduced pressure. The residue was dissolved in ethyl acetate (100 mmol), and a 4N solution of hydrogen chloride in ethyl acetate (3 ml) was added. The solvent was distilled off, and the residue was crystallized from ethanol-diethyl ether (10:1) to obtain methyl 4-(aminomethyl)phenylacetate hydrochloride (1.8 g, 8.35 mmol, 73%) as a colorless powder.

mp.198-210° C.

IR νₘₐₓ (KBr) cm⁻¹: 3300-2400 (br, NH₃⁺), 1736 (C═O).

¹H-NMR (D₂O) δ: 3.710 (3H, s), 3.776 (2H, s), 4.179 (2H, s), 7.369 (2H, d, J=8.4 Hz), 7.442 (2H, d, J=8.4 Hz).

(3) Diethyl cyanophosphonate (0.38 g, 2.30 mmol) was added to a solution of (3R,5S)-5-chloro-5-(2,3-dimethoxyphenyl)-1,2,3,5-tetrahydro-1-(3-hydroxy-2,2-dimethylpropyl)-2-oxo-4,1-benzoxazepine-3-acetic acid (1 g, 2.09 mmol) and methyl 4-(aminomethyl)phenylacetate hydrochloride (0.47 g, 2.20 mmol), which was obtained in Example 61-(2), in N,N-dimethylformamide (10 ml), followed by the addition of triethylamine (0.53 g, 5.23 mol). This mixture was stirred at room temperature for 30 minutes. The mixture was diluted with ethyl acetate (100 ml), washed with water, a 5% aqueous potassium hydrogen sulfate solution, an aqueous saturated sodium bicarbonate solution and saturated brine, dried with sodium sulfate, and concentrated under reduced pressure. The residue was purified by recrystallization from ethyl acetate-hexane (1:3) to obtain methyl 4-[[[3R,5S]-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenylacetate (1.33 g, 2.08 mmol, 100%) as a colorless powder.

mp.159-161° C.

[α]_{D}²²−198.9° (c=0.16, methanol)

IR νₘₐₓ (KBr) cm⁻¹: 3600-3200 (br, OH, NH), 1738, 1651 (C═O).

¹H-NMR (CDCl₃) δ: 0.637 (3H, s), 1.046 (3H, s), 2.687 (1H, dd, J=6.2, 14.6 Hz), 2.887 (1H, dd, J=7.2, 14.6 Hz), 3.136 (1H, t, J=11.0 Hz), 3.376 (1H, d, J=13.8 Hz), 3.54-3.64 (1H, m), 3.601 (3H, s), 3.621 (2H, s), 3.691 (3H, s), 3.890 (3H, s), 4.38-4.48 (4H, m), 6.10-6.20 (1H, br), 6.149 (1H, s), 6.612 (1H, s), 6.98-7.35 (9H, m).

Elementary analysis (C₃₄H₃₉N₂O₈Cl.0.3H₂O) Cal'd: C, 63.36; H, 6.19; N, 4.35 Found: C, 63.21; H, 6.03; N, 4.45

(4) A mixture of methyl 4-[[[3R,5S]-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenylacetate (1.2 g, 1.88 mmol) obtained in Example 61-(3), a 1N aqueous sodium hydroxide solution (4.1 ml) and ethanol (20 ml) was stirred at 60° C. for 1 hour. This was diluted with water (50 ml) and, after acidification, extracted with ethyl acetate (100 ml). This was washed with saturated brine, dried with sodium sulfate, and concentrated under the reduced pressure. The residue was purified by recrystallization from ethyl acetate-hexane (1:1) to obtain 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenylacetic acid (0.87 g, 1.39 mmol, 74%) as a colorless powder.

mp.129-132° C.

[α]_{D}²²−208.8° (c=0.21, methanol).

IR νₘₐₓ (KBr) cm⁻¹: 3600-2400 (br, COOH, OH), 1718, 1651 (C═O).

¹H-NMR (CDCl₃) δ: 0.634 (3H, s), 1.022 (3H, s), 2.684 (1H, dd, J=5.8, 14.2 Hz), 2.883 (1H, dd, J=7.6, 14.2 Hz), 3.142 (1H, d, J=12.0 Hz), 3.362 (1H, d, J=14.4 Hz), 3.516 (1H, d, J=12.0 Hz), 3.588 (3H, s), 3.621 (2H, s), 3.883 (3H, s), 4.36-4.45 (4H, m), 6.130 (1H, s), 6.23-6.33 (1H, br), 6.610 (1H, d, J=2.0 Hz), 6.95-7.40 (9H, m).

Elemental analysis (C₃₃H₃₇N₂O₈Cl.H₂O) Cal'd: C, 61.63; H, 6.11; N, 4.36 Found: C, 61.82; H, 6.18; N, 4.25

### EXAMPLE 62

4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenylacetic acid

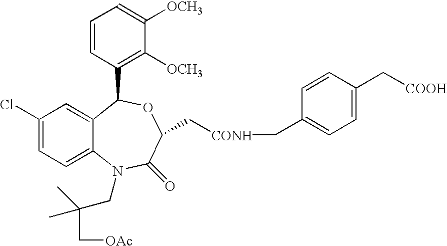

Acetyl chloride (3.5 g, 44.8 mmol) was added to a mixture of 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenylacetic acid (8 g, 12.8 mmol) obtained in Example 61-(4), pyridine (4.6 g, 57.6 mmol) and ethyl acetate (100 ml). The mixture was stirred at room temperature for 1 hour, water (4 ml) was added to this mixture, and the mixture was further stirred at room temperature for 2 hours. The organic layer was separated, washed with 1N hydrochloric acid and saturated brine. This was dried with sodium sulfate, and concentrated under reduced pressure. The residue was purified by silica gel column chromatography [eluent:ethyl acetate-methanol (20:1)]to obtain 4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenylacetic acid (4.5 g, 6.75 mmol, 53%) as a colorless amorphous powder.

[α]_{D}²²−149.9° (c=0.25, methanol).

IR νₘₐₓ (KBr) cm⁻: 3600-2400 (br, COOH), 1732, 1674 (C═O).

¹H-NMR (CDCl₃) δ: 0.925 (3H, s), 1.000 (3H, s), 2.020 (3H, s), 2.683 (1H, dd, J=5.8, 14.6 Hz), 2.874 (1H, dd, J=7.0, 14.6 Hz), 3.511 (1H, d, J=12.4 Hz), 3.596 (3H, s), 3.623 (2H, s), 3.709 (1H, d, J=10.6 Hz), 3.850 (1H, d, J=10.6 Hz), 3.881 (3H, s), 4.36-4.54 (4H, m), 6.238 (1H, s), 6.350 (1H, br), 6.627 (1H, d, J=2.2 Hz), 6.95-7.33 (9H, m).

### EXAMPLE 63

3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic Acid

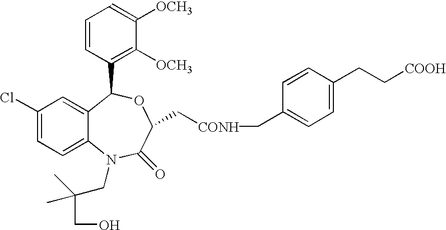

(1) 4-aminomethylbenzoic acid (10 g, 66.2 mmol) was dissolved in 1N NaOH (70 ml), and di-tert-butyl bicarbonate (16 g, 74.4 mmol) was added thereto at room temperature. This mixture was stirred at room temperature for 6 hours. The mixture was washed with ether, the aqueous layer was acidified, and extracted with ethyl acetate (100 ml) 2 times. The whole extract was washed with saturated brine, dried with sodium sulfate, and concentrated under the reduced pressure. The residue was purified by recrystallization from ethyl acetate-hexane (1:1) to obtain 4-(tert-butoxycarbonylaminomethyl)benzoic acid (13.4 g, 53.3 mmol, 81%) as a colorless powder.

mp.162-164° C.

IR νₘₐₓ (KBr) cm⁻¹: 3356 (NH), 3400-2400 (COOH), 1684 (C═O).

¹H-NMR (CDCl₃) δ: 1.471 (9H, s), 4.396 (2H, d, J=5.8), 4.90-5.05 (1H, br), 7.384 (2H, d, J=8.4 Hz), 8.069 (2H, d, J=8.4 Hz).

Elemental analysis (C₁₃H₁₇NO₄) Cal'd: C, 62.14; H, 6.82; N, 5.57 Found: C, 62.27; H, 6.60; N, 5.52

(2) Carbonyldiimidazole (9.5 g, 58.6 mmol) was added to a solution of 4-(tert-butoxycarbonylaminomethyl)benzoic acid (13.4 g, 53.3 mmol) obtained in Example 63-(1) in tetrahydrofuran (100 ml) at room temperature. After stirred at room temperature for 6 hours, a magnesium salt of malonic acid monoethyl ester (9.2 g, 32.0 mmol) was added. This mixture was stirred at room temperature overnight. The mixture was diluted with ethyl acetate (100 ml), washed with an aqueous saturated ammonium chloride 2 times, dried with sodium sulfate, and concentrated under the reduced pressure. The residue was purified by silica gel column chromatography [hexane-ethyl acetate (2:1)] to obtain ethyl 3-[4-(tert-butoxycarbonylaminomethyl)phenyl]-3-oxopropionate (17 g, 52.9 mmol, 99%) as a colorless oil.

IR νₘₐₓ (KBr) cm⁻¹: 3500-3300 (br, NH), 1738, 1720, 1687 (C═O).

¹H-NMR (CDCl₃) δ: 1.256 (3H, t, J=6.8 Hz), 1.462 (9H, s), 3.975 (6/7×2H, s), 4.209 (6/7×2H, q, J=6.8 Hz), 4.265 (6/7×2H, q, J=6.8 Hz), 4.377 (2H, d, J=5.4 Hz), 4.925 (1H, br), 5.649 (1/7×1H, s), 7.328 (1/7×2H, d, J=8.0 Hz), 7.387 (6/7×2H, d, J=8.0 Hz), 7.740 (1/7×2H, d, J=8.0 Hz), 7.912 (6/7×2H, d, J=8.0 Hz).

Elemental analysis (C₁₇H₂₃NO₅) Cal'd: C, 63.54; H, 7.21; N, 4.36 Found: C, 63.34; H, 7.14; N, 4.46

(3) Sodium borohydride (3 g, 79.3 mmol) was added to a solution of ethyl 3-[4-(tert-butoxycarbonylaminomethyl)phenyl]-3-oxopropionate (17 g, 52.9 mmol) obtained in Example 63-(2) in ethanol (200 ml) at 0° C. After stirred at room temperature for 30 minutes, the mixture was diluted with ethyl acetate (300 ml), and washed with water, a 5% aqueous potassium hydrogen sulfate solution, an aqueous saturated sodium bicarbonate solution and saturated brine. After dried with sodium sulfate, the residue was purified by silica gel column chromatography (eluent:hexane-ethyl acetate (2:1)] to obtain ethyl 3-[4-(tert-butoxycarbonylaminomethyl)phenyl]-3-hydroxypropionate (7.2 g, 22.3 mmol, 42%) as a colorless oil.

IR νₘₐₓ (KBr) cm⁻¹: 3600-3200 (br, OH, NH), 1714, 1693 (C═O).

¹H-NMR (CDCl₃) δ: 1.268 (3H, t, J=7.2 Hz), 1.458 (9H, s), 2.64-2.82 (2H, m), 4.187 (2H, q, J=7.2 Hz), 4.299 (2H, d, J=5.8 Hz), 4.80-4.90 (1H, br), 5.122 (1H, dd, J=5.2, 7.8 Hz), 7.25-7.40 (4H, m).

Elemental analysis (C₁₇H₂₅NO₅.0.2H₂O) Cal'd: C, 62.44; H, 7.83; N, 4.28 Found: C, 62.56; H, 7.64; N, 4.36

(4) A mixture of ethyl 3-[4-(tert-butoxycarbonylaminomethyl)phenyl]-3-hydroxypropionate (6.4 g, 19.8 mmol) obtained in Example 63-(3), triethylamine (2.4 g, 13.8 mmol), methanesulfonyl chloride (2.5 g, 21.8 mmol) and ethyl acetate (70 ml) was stirred at 0° C. for 30 minutes. 1,8-diazabicyclo[5.4.0]-7-undecene (3.3 g, 21.8 mmol) was added, and this mixture was stirred for 30 minutes. This mixture was diluted with ethyl acetate (100 ml), and washed with a 5% aqueous potassium hydrogen sulfate solution, an aqueous saturated sodium bicarbonate solution and saturated brine. The mixture was dried with sodium sulfate, and concentrated under reduced pressure. The residue was purified by silica gel column chromatography [eluent:hexane-ethyl acetate (3:1)] to obtain ethyl 3-[4-(tert-butoxycarbonylaminomethyl)phenyl]-2-propenoate (4.8 g, 15.7 mmol, 79%) as a colorless oil.

IR νₘₐₓ (KBr) cm⁻¹: 3354 (NH), 1712 (C═O).

¹H-NMR (CDCl₃) δ: 1.339 (3H, t, J=7.2 Hz), 1.462 (9H, s), 4.21-4.35 (4H, m), 4.82-4.96 (1H, br), 6.421 (1H, d, J=16.2 Hz), 7.302 (2H, d, J=8.2 Hz), 7.496 (2H, d, J=8.2 Hz), 7.671 (1H, d, J=16.2 Hz).

(5) 10% palladium carbon (0.3 g) was added to a solution of ethyl 3-[4-(tert-butoxycarbonylaminomethyl)phenyl]-2-propenoate (3.5 g, 11.5 mmol) obtained in Example 63-(4) in ethanol (100 ml). The mixture was subjected to normal pressure catalytic reduction at room temperature overnight, the catalyst was filtered to remove, and the filtrate was concentrated under the reduced pressure to obtain ethyl 3-[4-(tert-butoxycarbonylaminomethyl)phenyl]propionate (2.8 g, 9.11 mmol, 79%) as a colorless oil.

IR νₘₐₓ (KBr) cm⁻¹: 3354 (NH), 1714 (C═O).

¹H-NMR (CDCl₃) δ: 1.236 (3H, t, J=7.0 Hz), 1.458 (9H, s), 2.597 (2H, t, J=7.0 Hz), 2.934 (2H, t, J=7.0 Hz), 4.125 (2H, q, J=7.0 Hz), 3.277 (2H, d, J=5.8 Hz), 4.70-4.80 (1H, br), 7.14-7.23 (4H, m).

Elemental analysis (C₁₇H₂₅NO₄) Cal'd: C, 66.43; H, 8.20; N, 4.56 Found: C, 66.22; H, 7.99; N, 4.30

(6) A mixture of ethyl 3-[4-(tert-butoxycarbonylaminomethyl)phenyl]propionate (2.8 g, 9.11 mmol) obtained in Example 63-(5) and trifluoroacetic acid (10 ml) was stirred at room temperature for 10 minutes, and concentrated under the reduced pressure. The residue was dissolved in ethyl acetate (100 ml), and a 4N solution of hydrogen chloride in ethyl acetate (3 ml) was added. The solvent was distilled off, and the residue was crystallized from diethyl ether to obtain ethyl 3-[4-(aminomethyl)phenyl]propionate hydrochloride (1.8 g, 7.39 mmol, 81%) as a colorless powder.

mp.202-206° C.

IR νₘₐₓ (KBr) cm⁻¹: 3300-2400 (br, NH₃⁺), 1736 (C═O).

¹H-NMR (D₂O) δ: 1.130 (3H, t, J=7.4 Hz), 2.670 (2H, t, J=7.4 Hz), 2.923 (2H, t, J=7.4 Hz), 4.050 (2H, q, J=7.4 Hz), 4.110 (2H, s), 7.289 (2H, d, J=8.4 Hz), 7.356 (2H, d, J=8.4 Hz).

(7) Diethyl cyanophosphonate (0.37 g, 2.29 mmol) was added to a solution of (3R,5S)-7-chloro-5-(2,3-dimethoxyphenyl)-1,2,3,5-tetrahydro-1-(3-hydroxy-2,2-dimethylpropyl)-2-oxo-4,1-benzoxazepine-3-acetic acid (1 g, 2.09 mmol) and ethyl 3-[4-(aminomethyl)phenyl]propionate hydrochloride (0.53 g, 2.19 mmol), which was obtained in Example 63-(6), in N,N-dimethylformamide (10 ml), followed by the addition of triethylamine (0.58 g, 5.73 mol). The mixture was stirred at room temperature for 30 minutes. The mixture was diluted with ethyl acetate (100 ml), washed with water, a 5% aqueous potassium hydrogen sulfate solution, an aqueous saturated sodium bicarbonate solution and saturated brine, dried with sodium sulfate, and concentrated under the reduced pressure. The residue was purified by recrystallization from ethyl acetate-hexane (1:1) to obtain ethyl 3-[4-[[[(3R,5S)-7-chloro-5-(2,3-dimethoxyphenyl)-1-(3-hydroxy-2,2-diemthylpropyl)-2-oXo-1,2,3,5-tetrahydro-4,1-benzoxazepin-3-yl]acetyl]aminomethyl]phenyl]propionate (1.23 g, 1.84 mmol, 88%) as colorless prisms.

mp.172-174° C.

[α]_{D}²²−192.5° (c=0.18, methanol).

IR νₘₐₓ (KBr) cm⁻¹: 3600-3200 (br, OH, NH), 1732, 1658 (C═O).

¹H-NMR (CDCl₃) δ: 0.639 (3H, s), 1.048 (3H, s), 1.240 (3H, t, J=7.0 Hz), 2.601 (2H, t, J=7.0 Hz), 2.686 (1H, dd, J=5.8, 14.2 Hz), 2.876 (1H, dd, J=6.8, 14.2 Hz), 2.940 (2H, t, J=7.0 Hz), 3.05-3.19 (1H, m), 3.379 (1H, d, J=14.2 Hz), 3.54-3.64 (1H, m), 3.599 (3H, s), 3.892 (3H, s), 4.130 (2H, q, J=7.0 Hz), 4.35-4.51 (4H, m), 6.08-6.11 (1H, br), 6.150 (1H, s), 6.608 (1H, d, J=1.8 Hz), 6.96-7.41 (9H, m).

Elemental analysis (C₃₆H₄₃N₂O₈Cl) Cal'd: C, 64.81; H, 6.50; N, 4.20 Found: C, 64.59; H, 6.46; N, 4.34

(8) A mixture of ethyl 3-[4-[[[(3R,5S)-7-chloro-5-(2,3-dimethoxyphenyl)-1-(3-hydroxy-2,2-diemthylpropyl)-2-oxo-1,2,3,5-tetrahydro-4,1-benzoxazepin-3-yl]acetyl]aminomethyl]phenyl]propionate (1 g, 1.50 mmol) obtained in Example 63-(7), a 1N aqueous sodium hydride solution (3.5 ml) and ethanol (10 ml) was stirred at 60° C. for 1 hour. This was diluted with water (50 ml) and, after acidification, extracted with ethyl acetate (100 ml). This was washed with saturated brine, dried with sodium sulfate, and concentrated under reduced pressure. The residue was purified by silica gel column chromatography [eluent:ethyl acetate-methanol (2:1)] to obtain 3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid (0.76 g, 1.19 mmol, 79%) as a colorless amorphous powder.

[α]_{D}²²−182.7° (c=0.25, methanol).

IR νₘₐₓ (KBr) cm⁻¹: 3600-2400 (br, COOH, OH, NH), 1716, 1651 (C═O).

¹H-NMR (CDCl₃) δ: 0.641 (3H, s), 1.031 (3H, s), 2.641 (2H, t, J=7.2 Hz), 2.684 (1H, dd, J=6.0, 14.4 Hz), 2.874 (1H, dd, J=7.6, 14.4 Hz), 2.938 (2H, t, J=7.2 Hz), 3.147 (1H, d, J=11.6 Hz), 3.377 (1H, d, J=14.2 Hz), 3.579 (1H, d, J=11.6 Hz), 3.588 (3H, s), 3.885 (3H, s), 4.36-4.46 (4H, m), 6.131 (1H, s), 6.20-6.30 (1H, br), 6.603 (1H, d, J=2.0 Hz), 6.96-7.35 (9H, m).

Elemental analysis (C₃₄H₃₉N₂O₈Cl.0.5H₂O) Cal'd: C, 63.01; H, 6.22; N, 4.32 Found: C, 63.17; H, 6.42; N, 4.22

### EXAMPLE 64

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic Acid

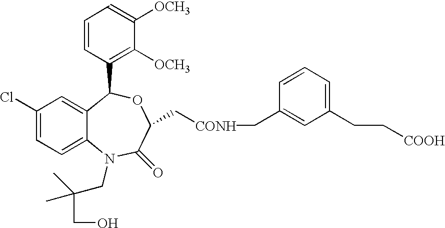

(1) Diethyl cyanophosphonate (0.41 g) and triethylamine (0.8 ml) were added to a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepine-3-acetic acid (1.0 g) and 3-[3-(aminomethyl)phenyl]propionic acid ethyl ester hydrochloride (0.56 g) in N,N-dimethylformamide (12 ml), and the mixture was stirred at room temperature for 30 minutes. The reaction solution was diluted by the addition of ethyl acetate (50 ml), washed successively with 5% potassium hydrogen sulfate, an saturated sodium bicarbonate and water, and dried with anhydrous sodium sulfate. The solvent was concentrated under the reduced pressure, and the residue was purified by silica gel column chromatography (eluent, hexane:ethyl acetate:methanol=30:20:1) to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid ethyl ester (1.15 g) as colorless crystals.

mp.94-95° C.

¹H-NMR (CDCl₃) δ: 0.640 (3H, s), 1.044 (3H, s), 1.235 (3H, t, J=7.2 Hz), 2.55-3.25 (7H, m), 3.385 (1H, d, J=14.2 Hz), 3.600 (3H, s), 3.888 (3H, s), 4.125 (2H, q, J=7.2 Hz), 4.26-4.52 (3H, m), 6.153 (1H, s), 6.607 (1H, d, J=1.8 Hz), 6.92-7.45 (9H, m).

(2) 1N sodium hydroxide (5 ml) was added to a solution of 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid ethyl ester (1.0 g) obtained in Example 64-(1) in tetrahydrofuran (5 ml) and methanol (10 ml), and the mixture was stirred at 60° C. for 40 minutes. The reaction solution was diluted by the addition of water (50 ml), extracted with ether, the aqueous layer was neutralized with 1N hydrochloric acid, and extracted with ethyl acetate. The organic layer was washed with water, and dried with anhydrous sodium sulfate. The solvent was concentrated under the reduced pressure, the crystals obtained from the residue were recrystallized from ethyl acetate and hexane to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid (0.82 g) as colorless crystals.

mp.177-178° C.

¹H-NMR (CDCl₃) δ: 0.647 (3H, s), 1.040 (3H, s), 2.55-3.05 (8H, m), 3.176 (1H, d, J=12.4 Hz), 3.395 (1H, d, J=14.4 Hz), 3.590 (3H, s), 3.594 (1H, d, J=12.4 Hz), 3.888 (3H, s), 4.22-4.57 (4H, m), 6.128 (1H, s), 6.17-6.32 (1H, m), 6.620 (1H, d, J=1.8 Hz), 6.94-7.45 (9H, m).

### EXAMPLE 65

3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic Acid

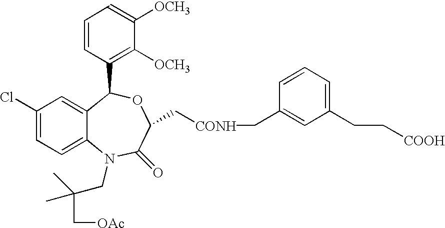

3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid (0.4 g) obtained in Example 64-(2) was reacted and treated according to the method of Example 54 to obtain 3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid (0.34 g) as a colorless amorphous powder.

¹H-NMR (CDCl₃) δ: 0.930 (3H, s), 0.998 (3H, s), 2.013 (3H, s), 2.57-2.98 (6H, m), 3.531 (1H, d, J=14.2 Hz), 3.596 (3H, s), 3.720 (1H, d, J=11.2 Hz), 3.851 (1H, d, J=11.2 Hz), 3.879 (3H, s), 4.25-4.47 (3H, m), 4.534 (1H, d, J=14.2 Hz), 6.244 (1H, s), 6.25-6.35 (1H, m), 6.623 (1H, br), 6.92-7.38 (9H, m)

### EXAMPLE 66

3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3,4-dimethoxyphenyl]propionic Acid

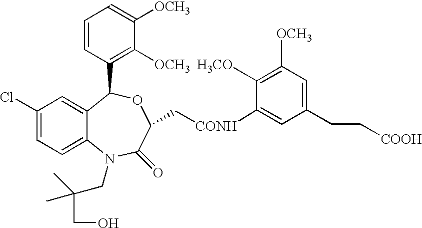

(1) A mixture of 5-nitrovanillin (10 g, 50.7 mmol), potassium carbonate (10.5 g, 76,1 mmol), iodomethane (7.9 g, 55.8 mmol) and N,N-dimethylformamide (100 ml) was stirred at 40° C. overnight. This mixture was diluted with water, and extracted with ethyl acetate (100 ml). The extract was washed with saturated brine, dried with anhydrous sodium sulfate, and concentrated under reduced pressure. The residue was recrystallized from ethyl acetate-hexane (1:2) to obtain 3,4-dimethoxy-5-nitrobenzaldehyde (5.1 g, 24.2 mmol, 47%) as colorless prisms.

IR νₘₐₓ (KBr) cm⁻¹: 1703 (C═O).

¹H-NMR (CDCl₃) δ: 4.005 (3H, s), 4.084 (3H, s), 7.628 (1H, t, J=1.8 Hz), 7.842 (1H, d, J=1.8 Hz), 9.923 (1H, s).

Elemental Analysis (C₉H₉NO₅) Cal'd: C, 51.19; H, 4.30; N, 6.63. Found: C, 51.24; H, 4.11; N, 6.57.

(2) A solution of triethylphosphonoacetic acid (5.9 g, 26.5 mmol) in tetrahydrofuran (20 ml) was added to a mixture of 3,4-dimethoxy-5-nitrobenzaldehyde (5.08 g, 24.1 mmol) obtained in Example 66-(1), sodium hydride (1.2 g, 48.2 mmol) and tetrahydrofuran (50 ml) at 0° C. After stirred at room temperature for 1 hour, the reaction was quenched with a 5% aqueous sodium hydrogen sulfate solution. The reaction was diluted with ethyl acetate (100 ml), washed with saturated brine, dried with anhydrous sodium sulfate, and concentrated under reduced pressure. The residue was purified by recrystallization from ethyl acetate-hexane (1:2) to obtain ethyl 3-(3,4-dimethoxy-5-nitrophenyl)-2-propenoate (2,7 g, 9.60 mmol, 40%) as pale yellow prisms.

mp.87-88° C.

IR νₘₐₓ (KBr) cm⁻¹: 1712 (C═O), 1643 (C═C).

¹H-NMR (CDCl₃) δ: 1.346 (3H, t, J=7.0 Hz), 3.962 (3H, s), 4.011 (3H, s), 4.280 (2H, q, J=7.0 Hz), 6.412 (1H, d, J=15.8 Hz), 7.214 (1H, d, J=1.8 Hz), 7.498 (1H, d, J=1.8 Hz), 7.594 (1H, d, J=15.8 Hz).

Elemental Analysis (C₁₃H₁₅NO₆) Cal'd: C, 55.51; H, 5.38; N, 4.98. Found: C, 55.32; H, 5.53; N, 4.93.

(3) 10% palladium carbon (0.2 g) was added to a solution of ethyl 3-(3,4-dimethoxy-5-nitrophenyl)-2-propenoate (2.7 g, 9.60 mmol) obtained in Example 66-(2) in ethanol (50 ml), and the mixture was stirred at room temperature and normal pressure for 5 hours under hydrogen atmosphere. The catalyst was filtered to remove, and the filtrate was concentrated under reduced pressure. The residue was dissolved in ethyl acetate (50 ml), and a 4N solution of hydrogen chloride in ethyl acetate (3 ml), which was concentrated under reduced pressure. The residue was washed with diethyl ether-hexane (1:1) to obtain ethyl 3-(5-amino-3,4-dimethoxyphenyl)-2-propionate hydrochloride (2.5 g, 8.63 mmol, 90%) as a colorless powder.

mp.158-166° C.

IR νₘₐₓ (KBr) cm⁻¹: 3400-2300 (br, NH⁺), 1732 (C═O).

¹H-NMR (D₂O) δ: 1.009 (3H, t, J=7.0 Hz), 2.562 (2H, t, J=7.4 Hz), 2.789 (2H, t, J=7.4 Hz), 3.742 (3H, s), 3.672, 3.769 (total 3H, each s), 3.966 (2H, q, J=7.0 Hz), 6.705 (1H, d, J=1.8 Hz), 6.896 (1H, s).

Elemental Analysis (C₁₃H₂₀NO₄Cl) Cal'd: C, 53.89; H, 6.96; N, 4.83. Found: C, 53.63; H, 6.96; N, 4.75.

(4) Thionyl chloride (0.7 g, 5.88 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1), N,N-dimethylformamide (0.03 ml) and tetrahydrofuran (10 ml) at room temperature, and the mixture was stirred for 1 hour. The residue obtained by concentration under reduced pressure was dissolved in tetrahydrofuran (5 ml). This solution was added to a mixture of ethyl 3-(5-amino-3,4-dimethoxyphenyl)-2-propionate hydrochloride (0.61 g, 2.11 mmol) obtained in Example 66-(3), triethylamine (0.48 g, 4.80 mmol) and tetrahydrofuran (10 ml). The mixture was stirred at room temperature for 30 minutes, and diluted with ethyl acetate (100 ml). This was washed with 1N hydrochloric acid, an aqueous saturated sodium bicarbonate solution and saturated brine, dried with anhydrous sodium sulfate, and concentrated under reduced pressure. The residue was purified by silica gel column chromatography [eluent: hexane-ethyl acetate (1:1)] to obtain ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3,4-dimethoxyphenyl]propionate (0.90 g, 1.19 mmol, 62%) as a colorless amorphous powder.

[α]_{D}²²−119.6° (c=0.15, methanol).

IR νₘₐₓ (KBr) cm⁻¹: 3400-3300 (br, NH), 1732, 1682 (C═O).

¹H-NMR (CDCl₃) δ: 0.947 (3H, s), 1.018 (3H, s), 1.249 (3H, t, J=7.2 Hz), 2.030 (3H, s), 2.55-2.63 (2H, m), 2.77-2.92 (3H, m), 3.082 (1H, dd, J=7.0, 14.2 Hz), 3.533 (1H, d, J=14.2 Hz), 3.610 (3H, s), 3.721 (1H, d, J=11.0 Hz), 3.792 (3H, s), 3.82-3.89 (7H, m), 4.136 (2H, q, J=7.2 Hz), 4.436 (1H, dd, J=6.2, 7.0 Hz), 4.572 (1H, d, J=14.2 Hz), 6.283 (1H, s), 6.511 (1H, d, J=1.8 Hz), 6.642 (1H, d, J=1.4 Hz), 6.94-7.33 (5H, m), 7.819 (1H, s), 8.241 (1H, s).

Elemental Analysis (C₃₉H₄₇N₂O₁₀Cl.0.5H₂O) Cal'd: C, 61.29; H, 6.33; N, 3.67. Found: C, 61.41; H, 6.48; N, 3.81.

(5) A mixture of ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3,4-dimethoxyphenyl]propionate (0.8 g, 1.06 mmol) obtained in Example 66-(4), a 1N aqueous sodium hydroxide (3 ml) and ethanol (8 ml) was stirred at 60° C. for 30 minutes. The mixture was diluted with water (50 ml) and, after acidification, extracted with ethyl acetate (100 ml). This was washed with saturated brine, dried with sodium sulfate, and concentrated under reduced pressure. The residue was purified by recrystallization from ethyl acetate-hexane (1:1) to obtain 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3,4-dimethoxyphenyl]propionic acid (0.40 g, 0.584 mmol, 55%) as colorless prisms.

mp.145-148° C.

[α]_{D}²²−158.5° (c=0.20, methanol).

IR νₘₐₓ (KBr) cm⁻¹: 3600-2400 (br, COOH, NH, OH), 1732, 1714, 1660 (C═O).

¹H-NMR (CDCl₃) δ: 0.645 (3H, s), 1.042 (3H, s), 2.643 (2H, t, J=7.2 Hz), 2.836 (1H, dd, J=5.4, 14.6 Hz), 2.885 (2H, t, J=7.2 Hz), 3.112 (1H, dd, J=7.4, 14.6 Hz), 3.156 (1H, d, J=11.6 Hz), 3.381 (1H, d, J=14.2 Hz), 3.610 (3H, s), 3.623 (1H, d, J=11.6 Hz), 3.797 (3H, s), 3.843 (3H, s), 3.891 (3H, s), 4.443 (1H, dd, J=5.4, 7.4 Hz), 4.471 (1H, d, J=14.2 Hz), 6.180 (1H, s), 6.524 (1H, d, J=1.8 Hz), 6.627 (1H, d, J=1.8 Hz), 6.96-7.36 (5H, m), 7.785 (1H, s), 8.246 (1H, s).

Elemental Analysis (C₃₅H₄₁N₂O₁₀Cl) Cal'd: C, 61.35; H, 6.03; N, 4.09. Found: C, 61.19; H, 6.34; N, 3.90.

### EXAMPLE 67

3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3,4-dimethoxyphenyl]propionic Acid

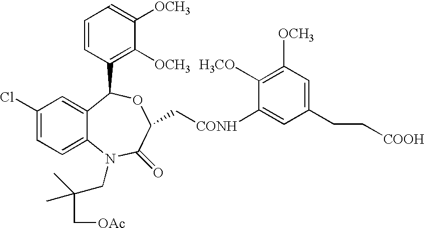

Acetyl chloride (80 mg, 1.02 mmol) was added to a mixture of 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3,4-dimethoxyphenyl]propionic acid (0.2 g, 0.292 mmol) obtained in Example 66-(5), pyridine (0.10 g, 1.32 mmol) and ethyl acetate (5 ml). After stirred at room temperature for 1 hour, water (4 ml) was added to this mixture, and the mixture was further stirred at room temperature for 2 hours. The organic layer was separated, and washed with 1N hydrochloric acid and saturated brine. This was dried with sodium sulfate, and concentrated under reduced pressure. The residue was purified by recrystallization from ethyl acetate-hexane (1:2) to obtain 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3,4-dimethoxyphenyl]propionic acid (0.17 g, 0.234 mmol, 80%) as a colorless amorphous powder.

[α]_{D}²²−138.0° (c=0.15, methanol).

IR νₘₐₓ (KBr) cm⁻¹: 3600-2400 (br, COOH, NH), 1732, 1682 (C═O).

¹H-NMR (CDCl₃) δ: 0.947 (3H, s), 1.015 (3H, s), 2.026 (3H, s), 2.645 (2H, t, J=7.8 Hz), 2.831 (1H, dd, J=5.6, 14.6 Hz), 2.883 (2H, t, J=7.8 Hz), 3.085 (1H, dd, J=7.0, 14.6 Hz), 3.534 (1H, d, J=14.4 Hz), 3.610 (3H, s), 3.7318 (1H, d, J=10.8 Hz), 3.78-3.89 (10H, m), 4.432 (1H, dd, J=5.6, 7.0 Hz), 4.571 (1H, d, J=14.4 Hz), 6.280 (1H, s), 6.522 (1H, s), 6.290 (1H, s), 6.647 (1H, s), 6.94-7.33 (5H, m), 7.821 (1H, s), 8.273 (1H, s).

Elemental Analysis (C₃₇H₄₃N₂O₁₁Cl) Cal'd: C, 61.11; H, 5.96; N, 3.85. Found: C, 60.79; H, 6.18; N, 3.52.

### EXAMPLE 68

4-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]butanoic acid

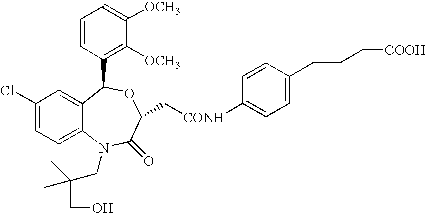

(1) Carbonyldiimidazole (8.7 g, 30.4 mmol) was added to a solution of 4-nitrophenylacetic acid (10 g, 55.2 mmol) in tetrahydrofuran (100 ml) at room temperature. After stirred at room temperature for 6 hours, a magnesium salt of malonic acid monoethyl ester (4.4 g, 15.2 mmol) was added. This mixture was stirred at 60° C. for 1.5 hours, The reaction solution was diluted with ethyl acetate (100 ml), washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (3:1)] to obtain ethyl 4-(4-nitrophenyl)-3-oxobutanoate (10.3 g, 41.0 mmol, 74%) as a pale yellow powder.

IR νₘₐₓ (KBr) cm⁻¹: 1738, 1722 (C═O).

¹H-NMR (CDCl₃) δ: 1.280 (1/7×3H, t, J=7.0 Hz), 1.289 (6/7×3H, t, J=7.0 Hz), 3.529 (6/7×2H, s), 3.603 (1/7×2H, s), 4.000 (6/7×2H, s), 4.194 (1/7×2H, q, J=7.0 Hz), 4.216 (6/7×2H, q, J=7.0 Hz), 4.973 (1/7×1H, s), 7.36-7.46 (2H, m), 8.17-8.24 (2H, m).

Elemental Analysis (C₁₂H₁₃NO₅) Cal'd: C, 57.37; H, 5.22; N, 5.58. Found: C, 57.42; H, 5.13; N, 5.72.

(2) Sodium borohydride (1.9 g, 49.2 mmol) was added to a solution of ethyl 4-(4-nitrophenyl)-3-oxobutanoate (10.3 g, 41.0 mmol) obtained in Example 68-(1) at −78° C. After stirred at −78° C. for 30 minutes, 1N hydrochloric acid (30 ml) was added. This mixture was diluted with ethyl acetate (300 ml), washed with water, an aqueous saturated sodium bicarbonate solution and saturated brine, dried with sodium sulfate, and the residue was purified by silica gel column chromatography [eluent: hexane-ethyl acetate (2:1)] to obtain ethyl 4-(4-nitrophenyl)-3-hydroxybutanoate (5.6 g, 22.0 mmol, 54%) as pale yellow prisms.

mp.71-72° C.

IR νₘₐₓ (KBr) cm⁻¹: 3600-3200 (br, OH), 1728 (C═O).

¹H-NMR (CDCl₃) δ: 1.273 (3H, t, J=7.4 Hz), 2.442 (1H, dd, J=8.0, 16.4 Hz), 2.552 (1H, dd, J=4.0, 16.4 Hz), 2.870 (1H, dd, J=5.6, 13.6 Hz), 2.952 (1H, dd, J=7.0, 13.6 Hz), 3.151 (1H, d, J=4.0 Hz), 4.177 (2H, q, J=7.4 Hz), 4.27-4.35 (1H, m), 7.415 (2H, d, J=8.4 Hz), 8.173 (2H, d, J=8.4 Hz).

Elemental Analysis (C₁₂H₁₅NO₅) Cal'd: C, 56.91; H, 5.97; N, 5.53. Found: C, 56.95; H, 6.26; N, 5.57.

(3) A mixture of ethyl 4-(4-nitrophenyl)-3-hydroxybutanoate (5.6 g, 22.0 mmol) obtained in Example 68-(2), triethylamine (2.7 g, 27.1 mmol), methanesulfonyl chloride (2.8 g, 24.2 mmol) and ethyl acetate (60 ml) was stirred at 0° C. for 30 minutes. 1,8-diazabicyclo[5.4.0]-7-undecene (7.4 g, 48.4 mmol) was added, and this mixture was stirred at 0° C. for 30 minutes. This mixture was diluted with ethyl acetate (100 ml), and washed with 1N hydrochloric acid (80 ml), an aqueous saturated sodium bicarbonate solution and saturated brine. The mixture was dried with sodium sulfate, and concentrated under reduced pressure. The residue was purified by silica gel column chromatography [eluent:hexane-ethyl acetate (2:1)] to obtain ethyl 4-(4-nitrophenyl)-2-butenoate (4.9 g, 20.8 mmol, 95%) as a yellow oil.

IR νₘₐₓ (KBr) cm⁻¹: 1732 (C═O), 1653 (C═C).

¹H-NMR (CDCl₃) δ: 1.297 (3H, t, J=7.2 Hz), 3.307 (2H, d, J=5.6 Hz), 4.199 (2H, q, J=7.2 Hz), 6.484 (1H, dd, J=5.6, 16.0 Hz), 6.590 (1H, d, J=16.0 Hz), 7.509 (2H, d, J=9.0 Hz), 8.182 (2H, d, J=9.0 Hz).

(4) 10% palladium carbon (0.2 g) was added to a solution of ethyl 4-(4-nitrophenyl)-2-butenoate (4.9 g, 20.8 mmol) obtained in Example 68-(3) in ethanol (60 ml). This suspension was stirred at room temperature and normal pressure for 5 hours under hydrogen atmosphere. The catalyst was filtered to remove, and the filtrate was concentrated under reduced pressure. The residue was diluted with ethyl acetate (50 ml), and a 4N solution of hydrogen chloride in ethyl acetate (6 ml) was added thereto. The solvent was distilled off, and the residue was washed with diethyl ether to obtain ethyl 4-(4-nitrophenyl)-2-butanoate hydrochloride (0.8 g, 3.28 mmol, 16%) as a yellow powder.

mp.129-137° C.

IR νₘₐₓ (KBr) cm⁻¹: 3200-2300 (br, NH⁺), 1720 (C═O).

¹H-NMR (D₂O) δ: 1.059 (3H, t, J=7.4 Hz), 1.787 (2H, quintet, J=7.8 Hz), 2.212 (2H, t, J=7.8 Hz), 2.551 (2H, t, J=7.8 Hz), 3.905 (2H, q, J=7.4 Hz), 7.168 (2H, d, J=8.8 Hz), 7.241 (2H, d, J=8.8 Hz).

Elemental Analysis (C₁₂H₁₈NO₂Cl) Cal'd: C, 59.13; H, 7.44; N, 5.75. Found: C, 58.86; H, 7.30; N, 5.76.

(5) Thionyl chloride (0.7 g, 5.88 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.5 g, 1.92 mmol) obtained in Example 1-(1) and N,N-dimethylformamide (0.03 ml) in tetrahydrofuran (10 ml) at room temperature. The mixture was stirred for 1 hour, and concentrated under reduced pressure. The residue was dissolved in tetrahydrofuran (5 ml), and added to a mixture of ethyl 4-(4-nitrophenyl)-2-butanoate hydrochloride (0.61 g, 2.11 mmol) obtained in Example 68-(4), triethylamine (0.48 g, 4.80 mmol) and tetrahydrofuran (10 ml). This was stirred at room temperature for 30 minutes, water was added, and tetrahydrofuran was distilled off. The residue was diluted with ethyl acetate (50 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 4-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]butanoate (1.1 g, 1.55 mmol, 81%) as a colorless amorphous powder.

[α]_{D}²²−122.3° (c=0.17 methanol).

IR νₘₐₓ (KBr) cm⁻¹: 3400-3200 (br, NH), 1732, 1682 (C═O).

¹H-NMR (CDCl₃) δ: 0.956 (3H, s), 1.022 (3H, s), 1.255 (3H, t, J=7.4 Hz), 1.920 (2H, quintet, J=7.0 Hz), 2.026 (3H, s), 2.302 (2H, t, J=7.0 Hz), 2.614 (2H, t, J=7.0 Hz), 2.809 (1H, dd, J=6.0, 14.4 Hz), 2.993 (1H, dd, J=7.6, 14.4 Hz), 3.532 (1H, d, J=13.8 Hz), 3.617 (3H, s), 3.728 (1H, d, J=11.0 Hz), 3.871 (1H, d, J=11.0 Hz), 3.894 (3H, s), 4.126 (2H, q, J=7.4 Hz), 4.409 (1H, dd, J=6.0, 7.6 Hz), 4.557 (1H, d, J=13.8 Hz), 6.295 (1H, s), 6.639 (1H, d, J=1.8 Hz), 6.96-7.43 (9H, m), 7.810 (1H, s).

Elemental Analysis (C₃₈H₄₅N₂O₉Cl) Cal'd: C, 64.35; H, 6.40; N, 3.95. Found: C, 64.12; H, 6.57; N, 3.96.

(6) A mixture of ethyl 4-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]butanoate (1.0 g, 1.41 mmol) obtained in Example 68-(5), a 1N aqueous sodium hydroxide solution (4 ml) and ethanol (8 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethanol-hexane (1:3) to obtain 4-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]butanoic acid (0.83 g, 1.30 mmol, 92%) as colorless prisms.

mp.194-195° C.

[α]_{D}²²−140.9° (c=0.15, methanol).

IR νₘₐₓ (KBr) cm⁻¹: 3600-2400 (br, COOH, NH, OH), 1707, 1653 (C═O).

¹H-NMR (CDCl₃) δ: 0.648 (3H, s), 1.048 (3H, s), 1.930 (2H, quintet, J=7.4 Hz), 2.352 (2H, t, J=7.4 Hz), 2.636 (2H, t, J=7.4 Hz), 2.823 (1H, dd, J=5.6, 14.0 Hz), 3.010 (1H, dd, J=7.4, 14.0 Hz), 3.173 (1H, d, J=12.6 Hz), 3.380 (1H, d, J=14.6 Hz), 3.610 (3H, s), 3.623 (1H, d, J=12.6 Hz), 3.892 (3H, s), 4.438 (1H, dd, J=5.6, 7.4 Hz), 4.469 (1H, d, J=14.6 Hz), 6.189 (1H, s), 6.617 (1H, d, J=1.8 Hz), 6.96-7.43 (9H, m), 7.78-7.84 (1H, br).

Elemental Analysis (C₃₄H₃₉N₂O₈Cl) Cal'd: C, 63.89; H, 6.15; N, 4.38 Found: C, 63.68; H, 6.07; N, 4.28

### EXAMPLE 69

4-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]butanoic acid

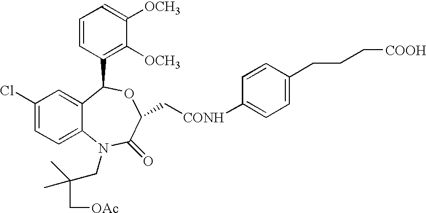

Acetyl chloride (86 mg, 1.10 mmol) was added to a mixture of 4-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]butanoic acid (0.2 g, 0.313 mmol) obtained in Example 68-(6), pyridine (0.17 g, 2.11 mmol) and ethyl acetate (5 ml). After stirred at room temperature for 1 hour, water (4 ml) was added to this mixture, and the mixture was further stirred at room temperature for 1 hour. The organic layer was separated, and washed with 1N hydrochloric acid and saturated brine. This was dried with sodium sulfate, and concentrated under reduced pressure to obtain 4-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]butanoic acid (0.18 g, 0.264 mmol, 84%) as a colorless amorphous powder.

[α]_{D}²²−128.5° (c=0.28, methanol).

IR νₘₐₓ (KBr) cm⁻¹: 3600-2400 (br, COOH, NH), 1732, 1682 (C═O).

¹H-NMR (CDCl₃) δ: 0.952 (3H, s), 1.015 (3H, s), 1.929 (2H, quintet, J=7.4 Hz), 2.020 (3H, s), 2.3524 (2H, t, J=7.4 Hz), 2.630 (2H, t, J=7.4 Hz), 2.814 (1H, dd, J=5.4, 14.0 Hz), 3.002 (1H, dd, J=7.4, 14.0 Hz), 3.527 (1H, d, J=14.4 Hz), 3.614 (3H, s), 3.726 (1H, d, J=11.0 Hz), 3.867 (1H, d, J=11.0 Hz), 3.889 (3H, s), 4.416 (1H, dd, J=5.4, 7.4 Hz), 4.551 (1H, d, J=14.4 Hz), 6.290 (1H, s), 6.637 (1H, d, J=2.0 Hz), 6.96-7.43 (9H, m), 7.933 (1H, s).

Elemental Analysis (C₃₆H₄₁N₂O₉Cl) Cal'd: C, 63.48; H, 6.07; N, 4.11 Found: C, 63.39; H, 6.32; N, 4.06

### EXAMPLE 70

4-[[(3R,5S)-7-chloro-5-(2,3-dimethoxyphenyl)-1-neopentyl-2-oxo-1,2,3,5-tetrahydro-4,1-benzoxazepin-3-yl]acetyl]aminobutanoic acid

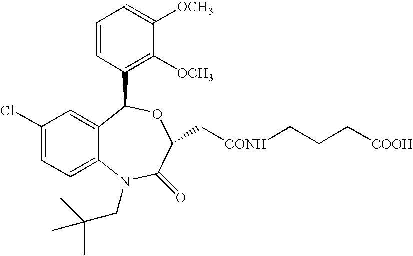

(1) Diethyl cyanophosphonate (0.19 g, 1.19 mmol) was added to a solution of (3R,5S)-7-chloro-5-(2,3-dimethoxyphenyl)-1-neopentyl-2-oxo-1,2,3,5-tetrahydro-4,1-benzoxazepine-3-acetic acid (0.5 g, 1.08 mmol) and methyl 4-aminobutanoate hydrochloride (0.17 g, 1.14 mmol) in N,N-dimethylformamide (5 ml) at room temperature, followed by the addition of triethylamine (0.27 g, 2.70 mmol). This mixture was stirred at room temperature for 30 minutes, and diluted with ethyl acetate (100 ml). This was washed with water, a 5% aqueous potassium hydrogen sulfate, an aqueous saturated sodium bicarbonate solution and saturated brine, dried with sodium sulfate, and concentrated under reduced pressure. The residue was purified by recrystallization from ethyl acetate-hexane (1:5) to obtain methyl 4-[[(3R,5S)-7-chloro-5-(2,3-dimethoxyphenyl)-1-neopentyl-2-oxo-1,2,3,5-tetrahydro-4,1-benzoxazepin-3-yl]acetyl]aminobutanoate (0.63 g, 1.12 mmol, 100%) as colorless prisms.

mp.74-75° C.

[α]_{D}²²−195.3° (c=0.21, methanol).

IR νₘₐₓ (KBr) cm⁻¹: 3400-3300 (br, NH), 1736, 1674 (C═O).

¹H-NMR (CDCl₃) δ: 0.941 (9H, s), 1.75-1.90 (2H, m), 2.344 (2H, t, J=7.2 Hz), 2.624 (1H, dd, J=5.8, 14.4 Hz), 2.821 (1H, dd, J=7.4, 14.4 Hz), 3.21-3.29 (2H, m), 3.355 (1H, d, J=14.0 Hz), 3.617 (3H, s), 3.665 (3H, s), 3.890 (3H, s), 4.379 (1H, dd, J=5.8, 7.4 Hz), 4.485 (1H, d, J=14.0 Hz), 5.95-6.18 (1H, br), 6.267 (1H, s), 6.608 (1H, s), 6.96-7.32 (5H, m).

Elemental Analysis (C₂₉H₃₇N₂O₇Cl) Cal'd: C, 61.48; H, 6.45; N, 5.12 Found: C, 61.34; H, 6.68; N, 4.97

(2) A mixture of methyl 4-[[(3R,5S)-7-chloro-5-(2,3-dimethoxyphenyl)-1-neopentyl-2-oxo-1,2,3,5-tetrahydro-4,1-benzoxazepin-3-yl]acetyl]aminobutanoate (0.75 g, 1.27 mmol) obtained in Example 70-(1), a 1N aqueous sodium hydroxide solution (2 ml) and ethanol (5 ml) was stirred at 60° C. for 30 minutes. This mixture was diluted with water (50 ml) and, after acidification, extracted with ethyl acetate (50 ml) 2 times. The whole organic layer was washed with saturated brine, dried with sodium sulfate, and concentrated under reduced pressure. The residue was purified by recrystallization from ethyl acetate-hexane (1:2) to obtain 3-[[(3R,5S)-7-chloro-5-(2,3-dimethoxyphenyl)-1-neopentyl-2-oxo-1,2,3,5-tetrahydro-4,1-benzoxazepin-3-yl]acetyl]aminobutanoic acid (0.38 g, 0.695 mmol, 91%) as colorless prisms

mp.128-130° C.

[α]_{D}²²−215.4° (c=0.16, methanol).

IR νₘₐₓ (KBr) cm⁻¹: 3400-2400 (br, COOH, NH), 1720, 1668 (C═O).

¹H-NMR (CDCl₃) δ: 0.938 (9H, s), 1.76-1.90 (2H, m), 2.364 (2H, t, J=6.8 Hz), 2.651 (1H, dd, J=5.6, 14.0 Hz), 2.853 (1H, dd, J=7.8, 14.0 Hz), 3.298 (2H, q, J=6.8 Hz), 3.361 (1H, d, J=14.0 Hz), 3.615 (3H, s), 3.888 (3H, s), 4.389 (1H, dd, J=5.6, 7.8 Hz), 4.476 (1H, d, J=14.0 Hz), 6.262 (1H, s), 6.28-6.38 (1H, br), 6.608 (1H, s), 6.95-7.33 (5H, m).

Elemental Analysis (C₂₈H₃₅N₂O₇Cl.0.5H₂O) Cal'd: C, 60.48; H, 6.53; N, 5.04 Found: C, 60.79; H, 6.35; N, 4.67

### EXAMPLE 71

3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methyphenyl]propionic Acid

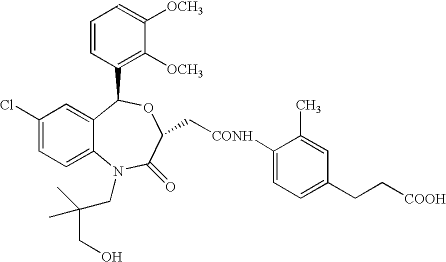

(1) Carbonyldiimidazole (14.8 g, 91.1 mmol) was added to a solution of 3-methyl-4-nitrobenzoic acid (15 g, 82.8 mmol) in tetrahydrofuran (150 ml) at room temperature. After stirred at room temperature for 6 hours, a magnesium salt of malonic acid monoethyl ester (13.1 g, 45.6 mmol) was added. This mixture was stirred at 60° C. for 1 hour, the reaction solution was diluted with ethyl acetate (100 ml), washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (10:1)] to obtain ethyl 3-(3-methyl-4-nitrophenyl)-3-oxopropionate (16.2 g, 64.5 mmol, 78%) as a colorless powder.

mp.48-50° C.

IR νₘₐₓ (KBr) cm⁻¹: 1741, 1693 (C═O)

¹H-NMR (CDCl₃) δ: 1.267 (⅖×3H, t, J=7.2 Hz), 1.350 (⅗×3H, t, J=7.2 Hz), 2.645 (3H, s), 4.009 (⅖×2H, s), 4.227 (⅖×2H, q, J=7.2 Hz), 4.330 (⅗×2H, q, J=7.2 Hz), 5.729 (⅗×1H, s), 7.68-8.04 (3H, m).

Elemental Analysis (C₁₂H₁₃NO₅,) Cal'd: C, 57.37; H, 5.22; N, 5.58 Found: C, 57.43; H, 5.19; N, 5.56

(2) Sodium borohydride (2.9 g, 77.4 mmol) was added to a solution of ethyl 3-(3-methyl-4-nitrophenyl)-3-oxopropionate (16.2 g, 64.5 mmol) obtained in Example 71-(1) in ethanol (160 ml) at −78° C. After stirred at −78° C. for 30 minutes, 6N hydrochloric acid (15 ml) was added. This mixture was diluted with ethyl acetate (200 ml), washed with water, an aqueous saturated sodium bicarbonate solution and saturated brine, dried with sodium sulfate, and the residue was purified by silica gel column chromatography [eluent:hexane-ethyl acetate (3:1)] to obtain ethyl 3-(3-methyl-4-nitrophenyl)-3-hydroxypropionate (7.9 g, 31.2 mmol, 48%) as a colorless oil.

IR νₘₐₓ (KBr) cm⁻¹: 3600-3300 (br, OH), 1732 (C═O).

¹H-NMR (CDCl₃) δ: 1.282 (3H, t, J=7.0 Hz), 2.619 (3H, s), 2.70-2.73 (2H, m), 3.602 (1H, d, J=3.4 Hz), 4.206 (2H, q, J=7.0 Hz), 5.13-5.21 (1H, m), 7.32-7.37 (2H, m), 7.984 (1H, d, J=8.2 Hz).

Elemental Analysis (C₁₂H₁₅NO₅) Cal'd: C, 56.91; H, 5.97; N, 5.583 Found: C, 56.79; H, 6.10; N, 5.50

(3) A mixture of ethyl 3-(3-methyl-4-nitrophenyl)-3-hydroxypropionate (7.7 g, 30.4 mmol) obtained in Example 71-(2), triethylamine (3.7 g, 36.5 mmol), methanesulfonyl chloride (3.8 g, 33.5 mmol) and ethyl acetate (80 ml) was stirred at 0° C. for 30 minutes. 1,8-diazabicyclo[5.4.0]-7-undecene (5.1 g, 33.5 mmol) was added, and this mixture was stirred at 0° C. for 30 minutes. This mixture was diluted with ethyl acetate (100 ml), and washed with 6N hydrochloric acid (20 ml), an aqueous saturated sodium bicarbonate solution and saturated brine. The mixture was dried with sodium sulfate, and concentrated under reduced pressure. The residue was purified by recrystallization from ethyl acetate-hexane (1:10) to obtain ethyl 3-(3-methyl-4-nitrophenyl)-2-propenoate (6.0 g, 25.5 mmol, 84%) as pale yellow needles.

mp.90-92° C.

IR νₘₐₓ (KBr) cm⁻¹: 1712 (C═O).

¹H-NMR (CDCl₃) δ: 1.352 (3H, t, J=7.4 Hz), 2.632 (3H, s), 4.289 (2H, q, J=7.4 Hz), 6.520 (1H, d, J=16.0 Hz), 7.46-7.50 (2H, m), 7.651 (1H, d, J=16.0 Hz), 7.98-8.03 (1H, m).

Elemental Analysis (C₁₂H₁₃NO₄) Cal'd: C, 61.27; H, 5.57; N, 5.95. Found: C, 61.15; H, 5.67; N, 5.94

(4) 10% palladium carbon (0.5 g) was added to a solution of ethyl 3-(3-methyl-4-nitrophenyl)-2-propenoate (5.8 g, 24.7 mmol) obtained in Example 71-(3) in ethanol (100 ml). This suspension was subjected to catalytic reduction at room temperature and normal pressure for 6 hours under hydrogen atmosphere. The catalyst was filtered to remove, and the filtrate was concentrated under reduced pressure. The residue was diluted with ethyl acetate (50 ml), and a 4N solution of hydrogen chloride in ethyl acetate (8 ml) was added. The solvent was distilled off, and the residue was washed with ethyl acetate-hexane (1:1) to obtain ethyl 3-(4-amino-3-methylphenyl)propionate hydrochloride (5.9 g, 24.2 mmol, 98%) as a colorless powder.

mp.158-163° C.

IR νₘₐₓ (KBr) cm⁻¹: 3200-2300 (br, NH₃⁺), 1722 (C═O).

¹H-NMR (D₂O) δ: 0.759 (3H, t, J=7.0 Hz), 1.942 (3H, s), 2.308 (2H, t, J=7.4 Hz), 2.550 (2H, t, J=7.4 Hz), 3.692 (2H, q, J=7.0 Hz), 6.78-6.91 (3H, m).

Elemental Analysis (C₁₂H₁₇NO₂.HCl) Cal'd: C, 59.13; H, 7.44; N, 5.75 Found: C, 58.94; H, 7.17; N, 5.58

(5) Thionyl chloride (1.4 g, 11.8 mmol) was added to a solution of (3R,5S)-1-(3-acetoxy-2,2-dimetylpropyl)-7-chloro-5-(2,3-dimethoxyphenyl)-2-oxo-1,2,3,5-tetrahydro-4,1-benzoxazepine-3-acetic acid (2.0 g, 3.85 mmol) obtained in Example 1-(1) and N,N-dimethylformamide (0.05 ml) in tetrahydrofuran (20 ml) at room temperature. The mixture was stirred for 1 hour, and concentrated under reduced pressure. The residue was dissolved in tetrahydrofuran (10 ml), which was added to a mixture of ethyl 3-(4-amino-3-methylphenyl)propionate hydrochloride (0.93 g, 4.51 mmol) obtained in Example 71-(4), dimethylaminopyridine (0.60 g, 4.95 mmol) and tetrahydrofuran (20 ml). This was stirred at room temperature for 30 minutes, water was added, and tetrahydrofuran was distilled off. The residue was diluted with ethyl acetate (100 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methylphenyl]propionate (2.08 g, 2.93 mmol, 76%) as a colorless amorphous powder.

[α]_{D}²²−145.3° (c=0.26, methanol).

IR νₘₐₓ (KBr) cm⁻¹: 3321 (NH), 1732, 1682 (C═O).

¹H-NMR (CDCl₃) δ: 0.956 (3H, s), 1.022 (3H, s), 1.240 (3H, t, J=7.4 Hz), 2.028 (3H, s), 2.198 (3H, s), 2.572 (2H, t, J=7.0 Hz), 2.809 (1H, d, J=5.2, 14.2 Hz), 2.879 (2H, q, J=7.0 Hz), 3.046 (1H, dd, J=7.6, 14.2 Hz), 3.533 (1H, d, J=14.2 Hz), 3.614 (3H, s), 3.722 (1H, d, J=11.0 Hz), 3.873 (1H, d, J=11.0 Hz), 3.892 (3H, s), 4.124 (2H, q, J=7.4 Hz), 4.400 (1H, dd, J=5.2, 7.6 Hz), 4.556 (1H, d, J=14.2 Hz), 6.290 (1H, s), 6.644 (1H, d, J=2.0 Hz), 6.96-7.37 (7H, m), 7.66-7.75 (2H, m).

Elemental Analysis (C₃₅H₄₅N₂O₉Cl) Cal'd: C, 64.35; H, 6.40; N, 3.95 Found: C, 64.08; H, 6.41; N, 3.71

(6) A mixture of ethyl 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methylphenyl]propionate (1.9 g, 2.68 mmol) obtained in Example 71-(5), a 1N aqueous sodium hydroxide solution (6 ml) and ethanol (20 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethanol to obtain 3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methyphenyl]propionic acid (1.35 g, 2.11 mmol, 79%) as a colorless powder.

[α]_{D}²²−169.5° (c=0.17, methanol).

IR νₘₐₓ (KBr) cm⁻¹: 3600-2400 (br, COOH, NH, OH), 1741, 1680 (C═O).

¹H-NMR (CDCl₃) δ: 0.670 (3H, s), 1.044 (3H, s), 2.205 (3H, s), 2.568 (2H, t, J=7.8 Hz), 2.81-2.92 (3H, m), 3.01-3.18 (2H, m), 3.408 (1H, d, J=14.2 Hz), 3.605 (3H, s), 3.611 (1H, d, J=11.0 Hz), 3.900 (3H, s), 4.43-4.50 (2H, m), 6.193 (1H, s), 6.618 (1H, s), 6.99-7.35 (7H, m), 7.587 (1H, d, J=8.8 Hz), 7.995 (1H, s).

Elemental Analysis (C₃₄H₃₉N₂O₈Cl) Cal'd: C, 63.89; H, 6.15; N, 4.38 Found: C, 63.93; H, 6.22; N, 4.20

### EXAMPLE 72

3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methyphenyl]propionic Acid

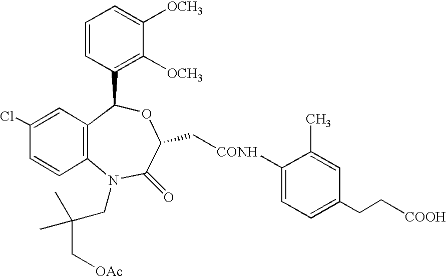

Acetyl chloride (86 mg, 1.10 mmol) was added to a mixture of 3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methyphenyl]propionic acid (0.2 g, 0.313 mmol) obtained in Example 71-(6), pyridine (0.11 g, 1.41 mmol) and ethyl acetate (3 ml). After stirred at room temperature for 1 hour, water (3 ml) was added to this mixture, and the mixture was further stirred at room temperature for 1 hour. The organic layer was separated, and washed with 1N hydrochloric acid and saturated brine. This was dried with sodium sulfate, and concentrated under reduced pressure to obtain 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methyphenyl]propionic acid (0.16 g, 0.242 mmol, 77%) as a colorless amorphous powder.

[α]_{D}²²−145.5° (c=0.22, methanol).

IR νₘₐₓ (KBr) cm⁻¹: 3400-2400 (br, COOH, NH), 1732, 1682 (C═O).

¹H-NMR (CDCl₃) δ: 0.954 (3H, s), 1.018 (3H, s), 2.026 (3H, s), 2.191 (3H, s), 2.628 (2H, t, J=7.5 Hz), 2.816 (1H, dd, J=5.4, 14.0 Hz), 2.883 (2H, t, J=7.5 Hz), 3.080 (1H, dd, J=7.6, 13.8 Hz), 3.531 (1H, d, J=14.2 Hz), 3.614 (3H, s), 3.721 (1H, d, J=11.0 Hz), 3.871 (1H, d, J=11.0 Hz), 3.892 (3H, s), 4.408 (1H, dd, J=5.4, 7.6 Hz), 4.550 (1H, d, J=14.2 Hz), 6.286 (1H, s), 6.645 (1H, d, J=1.8 Hz), 6.97-7.36 (7H, m), 7.69-7.75 (2H, br).

Elemental Analysis (C₃₆H₄₁N₂O₉Cl) Cal'd: C, 63.48; H, 6.07; N, 4.11 Found: C, 63.27; H, 6.42; N, 3.81

### EXAMPLE 73

3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methoxyphenyl]propionic Acid

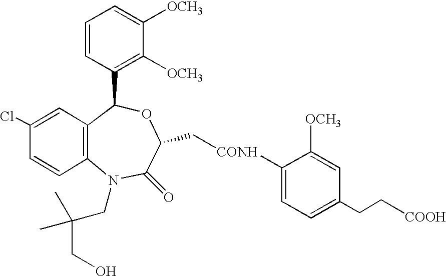

(1) Carbonyldiimidazole (4.5 g, 27.9 mmol) was added to a solution of 3-methoxy-4-nitrobenzoic acid (5 g, 25.4 mmol) in tetrahydrofuran (50 ml) at room temperature. After stirred at room temperature for 6 hours, a magnesium salt of malonic acid monoethyl ester (4.7 g, 27.9 mmol) was added. This mixture was stirred at 60° C. for 1 hour, the reaction solution was diluted with ethyl acetate (100 ml), washed with 1N hydrochloric acid, an aqueous saturated sodium bicarbonate solution and saturated brine, dried with sodium sulfate, and concentrated under reduced pressure. The residue was purified by silica gel column chromatography [eluent:hexane-ethyl acetate (3:1)] to obtain ethyl 3-(3-methoxy-4-nitrophenyl)-3-oxopropionate (5.7 g, 21.3 mmol, 84%) as colorless needles.

mp.94-95° C.

IR νₘₐₓ (KBr) cm⁻¹: 1741, 1693 (C═O).

¹H-NMR (CDCl₃) δ: 1.269 (½×3H, t, J=7.4 Hz), 1.355 (½×3H, t, J=7.4 Hz), 4.007 (½×2H, s), 4.022 (3H, s), 4.227 (½×2H, q, J=7.4 Hz), 4.300 (½×2H, q, J=7.4 Hz), 5.727 (½×1H, s), 7.35-7.90 (3H, m).

Elemental Analysis (C₁₂H₁₃NO₆) Cal'd: C, 53.93; H, 4.90; N, 5.24 Found: C, 53.81; H, 4.87; N, 5

(2) Sodium borohydride (0.97 g, 25.6 mmol) was added to a solution of ethyl 3-(3-methoxy-4-nitrophenyl)-3-oxopropionate (5.7 g, 21.3 mmol) obtained in Example 73-(1) in ethanol (60 ml) at −30° C. After stirred at 0° C. for 30 minutes, 6N hydrochloric acid (15 ml) was added. This mixture was diluted with ethyl acetate (100 ml), washed with water, an aqueous saturated sodium bicarbonate solution and saturated brine, dried with sodium sulfate, and the residue was purified by silica gel column chromatography [eluent:hexane-ethyl acetate (1:1)] to obtain ethyl 3-(3-methoxy-4-nitrophenyl)-3-hydroxypropionate (4.3 g, 16.0 mmol, 76%) as a colorless powder.

mp.54-56° C.

IR νₘₐₓ (KBr) cm⁻¹: 3600-3200 (br, OH), 1732 (C═O)

¹H-NMR (CDCl₃) δ: 1.288 (3H, t, J=7.2 Hz), 2.61-2.80 (2H, m), 3.663 (1H, d, J=3.6 Hz), 3.986 (3H, s), 4.212 (2H, q, J=7.2 Hz), 5.14-5.22 (1H, m), 6.972 (1H, d, J=8.4 Hz), 7.205 (1H, s), 7.855 (1H, d, J=8.4 Hz).

Elemental Analysis (C₁₂H₁₁NO₆) Cal'd: C, 53.53; H, 5.62; N, 5.26 Found: C, 53.54; H, 5.69; N, 5.12

(3) A mixture of ethyl 3-(3-methoxy-4-nitrophenyl)-3-hydroxypropionate (4.1 g, 15.2 mmol) obtained in Example 73-(2), triethylamine (1.8 g, 18.3 mmol), methanesulfonyl chloride (1.9 g, 16.8 mmol) and ethyl acetate (50 ml) was stirred at 0° C. for 30 minutes. 1,8-diazabicyclo[5.4.0]-7-undecene (2.6 g, 16.8 mmol) was added, and this mixture was stirred at 0° C. for 30 minutes. This mixture was diluted with ethyl acetate (100 ml), and washed with 6N hydrochloric acid (20 ml), an aqueous saturated sodium bicarbonate solution and saturated brine. The mixture was dried with sodium sulfate, and concentrated under reduced pressure. The residue was purified by recrystallization from ethyl acetate-hexane (1:2) to obtain ethyl 3-(3-methoxy-4-nitrophenyl)-2-propenoate (3.0 g, 11.9 mmol, 79%) as pale yellow needles.

mp.119-120° C.

IR νₘₐₓ (KBr) cm⁻¹: 1716 (C═O), 1606 (C═C).

¹H-NMR (CDCl₃) δ: 1.355 (3H, t, J=7.4 Hz), 4.004 (3H, s), 4.296 (2H, q, J=7.4 Hz), 6.518 (1H, d, J=15.8 Hz), 7.18 -7.21 (2H, m), 7.651 (1H, d, J=15.8 Hz), 7.879 (1H, d, J=8.8 Hz).

Elemental Analysis (C₁₂H₁₃NO₅) Cal'd: C, 57.37; H, 5.22; N, 5.58 Found: C, 57.26; H, 5.14; N, 5.34

(4) 10% palladium carbon (0.3 g) was added to a solution of ethyl 3-(3-methoxy-4-nitrophenyl)-2-propenoate (2.9 g, 11.5 mmol) obtained in Example 73-(3) in ethanol (60 ml). This suspension was subjected to catalytic reduction at room temperature and normal pressure for 5 hours under hydrogen atmosphere. The catalyst was filtered to remove, and filtrate was concentrated under reduced pressure. The residue was diluted with ethyl acetate (50 ml), and a 4N solution of hydrogen chloride in ethyl acetate (4 ml) was added. The solvent was distilled off, and the residue was washed with ethyl acetate-hexane (1:1) to obtain ethyl 3-(4-amino-3-methoxyphenyl)propionate hydrochloride (2.4 g, 9.24 mmol, 80%) as a colorless powder.

mp.157-163° C.

IR νₘₐₓ (KBr) cm⁻¹: 3200-2400 (br, NHa+), 1728 (C═O).

¹H-NMR (CDCl₃) δ: 1.078 (3H, t, J=7.4 Hz), 2.652 (2H, t, J=7.4 Hz), 2.899 (2H, t, J=7.4 Hz), 3.839 (3H, s), 4.010 (2H, q, J=7.4 Hz), 6.846 (1H, q, J=8.0 Hz), 6.996 (1H, s), 7.209 (1H, q, J=8.0 Hz).

Elemental Analysis (C₁₂H₁₇NO₃.HCl) Cal'd: C, 55.49; H, 6.99; N, 5.39 Found: C, 55.55; H, 7.09; N, 5.22

(5) Thionyl chloride (1.4 g, 11.8 mmol) was added to a solution of (3R,5S)-1-(3-acetoxy-2,2-dimetylpropyl)-7-chloro-5-(2,3-dimethoxyphenyl)-2-oxo-1,2,3,5-tetrahydro-4,1-benzoxazepine-3-acetic acid (2.0 g, 3.85 mmol) obtained in Example 1-(1) and N,N-dimethylformamide (0.05 ml) in tetrahydrofuran (20 ml) at room temperature. The mixture was stirred for 1 hour, and concentrated under reduced pressure. The residue was dissolved in tetrahydrofuran (10 ml), which was added to a mixture of ethyl 3-(4-amino-3-methoxyphenyl)propionate (1.2 g, 4.51 mmol) obtained in Example 73-(4), dimethylaminopyridine (0.60 g, 4.95 mmol) and tetrahydrofuran (20 ml). This was stirred at room temperature for 30 minutes, water was added, and tetrahydrofuran was distilled off. The residue was diluted with ethyl acetate (100 ml). This was washed with 1N hydrochloric acid, an aqueous saturated sodium bicarbonate solution and saturated brine, dried with sodium sulfate, and concentrated under reduced pressure. The residue was purified by silica gel column chromatography [eluent: hexane-ethyl acetate (1:1)] to obtain ethyl 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxyphenyl]propionate (1.3 g, 1.79 mmol, 47%) as a colorless amorphous powder.

[α]_{D}²²−145.3° (c=0.26, methanol)

IR νₘₐₓ (KBr) cm⁻: 3337 (NH), 1732, 1682 (C═O).

¹H-NMR (CDCl₃) δ: 0.949 (3H, s), 1.016 (3H, s), 1.244 (3H, t, J=7.4 Hz), 2.026 (3H, s), 2.594 (2H, t, J=7.5 Hz), 2.844 (1H, d, J=6.4, 14.6 Hz), 2.907 (2H, t, J=7.5 Hz), 3.015 (1H, dd, J=6.4, 14.6 Hz), 3.534 (1H, d, J=14.4 Hz), 3.607 (3H, s), 3.717 (1H, d, J=11.0 Hz), 3.788 (3H, s), 3.865 (1H, d, J=11.0 Hz), 3.889 (3H, s), 4.127 (2H, q, J=7.4 Hz), 4.444 (1H, t, J=6.4 Hz), 4.568 (1H, d, J=14.4 Hz), 6.286 (1H, s), 6.627 (1H, s), 6.93-6.78 (2H, m), 6.94-7.33 (5H, m), 8.124 (1H, s), 8.204 (1H, d, J=8.0 Hz).

Elemental Analysis (C₃₅H₄₅N₂O₁Cl) Cal'd: C, 62.93; H, 6.25; N, 3.86 Found: C, 62.57; H, 6.46; N, 3.58

(6) A mixture of ethyl 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxyphenyl]propionate (1.2 g, 1.65 mmol) obtained in Example 73-(5), a 1N aqueous sodium hydroxide solution (4 ml) and ethanol (10 ml) was stirred at 60° C. for 30 minutes. This was diluted with water (50 ml) and, after acidification, extracted with ethyl acetate (50 ml) 2 times. This was washed with saturated brine, dried with sodium sulfate, and concentrated under reduced pressure. The residue was purified by recrystallization from ethanol to obtain 3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methoxyphenyl]propionic acid (0.85 g, 1.30 mmol, 79%) as colorless prisms.

[α]_{D}²²−196.7° (c=0.14, methanol).

IR νₘₐₓ (KBr) cm⁻¹: 3600-2400 (br, COOH, NH, OH), 1712, 1691, 1651 (C═O).

¹H-NMR (CDCl₃) δ: 0.667 (3H, s), 1.038 (3H, s), 2.586 (2H, t, J=7.6 Hz), 2.852 (1H, dd, J=6.0, 14.6 Hz), 2.907 (2H, t, J=7.6 Hz), 3.046 (1H, dd, J=6.6, 14.6 Hz), 3.148 (1H, brd, J=11.4 Hz), 3.407 (1H, d, J=14.6 Hz), 3.603 (3H, s), 3.606 (1H, d, J=11.4 Hz), 3.808 (3H, s), 3.892 (3H, s), 4.442 (1H, dd, J=6.0, 6.6 Hz), 4.473 (1H, d, J=14.6 Hz), 6.187 (1H, s), 6.604 (1H, s), 6.75-7.36 (7H, m), 8.13-8.18 (2H, m).

Elemental Analysis (C₃₄H₃₉N₂O₉Cl.0.5H₂O) Cal'd: C, 61.49; H, 6.07; N, 4.22 Found: C, 61.70; H, 6.25; N, 3.96

### EXAMPLE 74

3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxyphenyl]propionic Acid

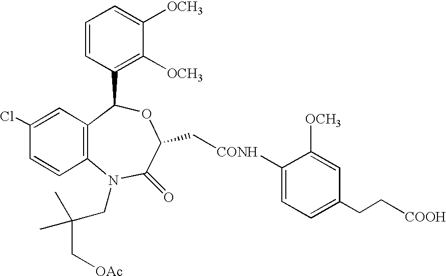

Acetyl chloride (63 mg, 0.801 mmol) was added to a mixture of 3-[4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methoxyphenyl]propionic acid (0.15 g, 0.229 mmol) obtained in Example 73-(6), pyridine (81 mg, 1.03 mmol) and ethyl acetate (3 ml). After stirred at room temperature for 1 hour, water (4 ml) was added to this mixture, and the mixture was further stirred at room temperature for 1 hour. The organic layer was separated, and washed with 1N hydrochloric acid and saturated brine. This was dried with sodium sulfate, and concentrated under reduced pressure to obtain 3-[4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxyphenyl]propionic acid (0.11 g, 0.158 mmol, 69%) as a colorless amorphous powder.

[α]_{D}²²−176.2° (c=0.19, methanol)

IR νₘₐₓ (KBr) cm⁻¹: 3600-2400 (br, COOH, NH), 1732, 1682 (C═O).

¹H-NMR (CDCl₃) δ: 0.949 (3H, s), 1.015 (3H, s), 2.022 (3H, s), 2.652 (2H, t, J=7.5 Hz), 2.847 (1H, dd, J=6.6, 15.0 Hz), 2.914 (2H, t, J=7.5 Hz), 3.017 (1H, dd, J=6.6, 15.0 Hz), 3.533 (1H, d, J=14.0 Hz), 3.604 (3H, s), 3.717 (1H, d, J=11.0 Hz), 3.778 (3H, s), 3.867 (1H, d, J=11.0 Hz), 3.885 (3H, s), 4.441 (1H, dd, J=6.0, 6.6 Hz), 4.566 (1H, d, J=14.0 Hz), 6.287 (1H, s), 6.634 (1H, d, J=1.4 Hz), 6.70-7.33 (7H, m), 8.152 (1H, s), 8.211 (1H, d, J=8.2 Hz).

Elemental Analysis (C₃₆H₄₁N₂O₁₀Cl) Cal'd: C, 62.02; H, 5.93; N, 4.02 Found: C, 62.06; H, 5.94; N, 3.69

### EXAMPLE 75

4-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethylbenzoic acid

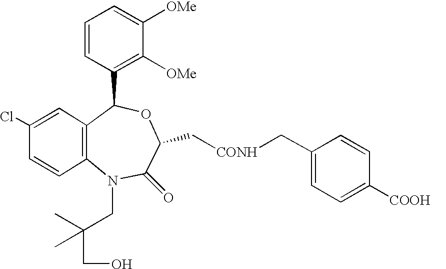

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid [JP 09-136880 A, Example 11-(4)] (1 g, 2.09 mmol) and methyl 4-(aminomethyl)benzoate hydrochloride (0.46 g, 2.30 mmol) in N,N-dimethylformamide (10 ml) were added diethyl cyanophosphate (0.38 g, 2.30 mmol) and then triethylamine (0.53 g, 5.23 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by column chromatography [eluent:hexane-ethyl acetate (1:6)] and recrystallized from ethyl acetate-hexane (1:1) to obtain methyl 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethylbenzoate (0.84 g, 1.34 mmol, 64%) as a colorless powder.

Melting point 110-112° C.

[α]_{D}²²−194.7° (c=0.23, MeOH).

IR νₘₐₓ (KBr) cm⁻¹: 1720, 1651 (C═O).

¹H-NMR (CDCl₃) δ: 0.637 (3H, s), 1.046 (3H, s), 2.724 (1H, dd, J=6.2, 14.4 Hz), 2.907 (1H, dd, J=6.8, 14.4 Hz), 3.08-3.19 (1H, m), 3.372 (1H, d, J=14.0 Hz), 3.56-3.64 (1H, m), 3.594 (3H, s), 3.890 (3H, s), 3.918 (3H, s), 4.40-4.52 (4H, m), 6.149 (1H, s), 6.284 (1H, br), 6.608 (1H, d, J=1.8 Hz), 6.96-7.35 (7H, m), 7.984 (2H, d, J=7.8 Hz).

Elemental analysis (C₃₃H₃₇N₂O₈Cl.0.8H₂O) Cal'd: C, 61.98; H, 6.08; N, 4.38. Found: C, 62.07; H, 6.24; N, 4.14.

(2) A mixture of methyl 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethylbenzoate obtained in Example 75-(1) (0.74 g, 1.18 mmol), 1 N aqueous sodium hydroxide solution (2.5 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate to obtain 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethylbenzoic acid (0.44 g, 0.720 mmol, 61%) as a colorless powder.

Melting point 143-144° C.

[α]_{D}²²−213.8° (c=0.27, MeOH).

IR νₘₐₓ (KBr) cm⁻¹: 3600-2400 (br, COOH, OH), 1709, 1653 (C═O).

¹H-NMR (CDCl₃) δ: 0.645 (3H, s), 1.051 (3H, s), 2.747 (1H, dd, J=5.6, 14.4 Hz), 2.927 (1H, dd, J=6.6, 14.4 Hz), 3.386 (1H, d, J=14.0 Hz), 3.597 (3H, s), 3.599 (1H, d, J=11.8 Hz), 3.891 (3H, s), 4.42-4.53 (4H, m). 6.153 (1H, s), 6.400 (1H, br), 6.611 (1H, d, J=2.0 Hz), 6.96-7.36 (7H, m), 8.018 (2H, d, J=8.2 Hz).

Elemental analysis (C₃₂H₃₅N₂O₈Cl.0.2H₂O) Cal'd: C, 62.53; H, 5.80; N, 4.56. Found: C, 62.44; H, 5.81; N, 4.18.

### EXAMPLE 76

4-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethylbenzoic acid

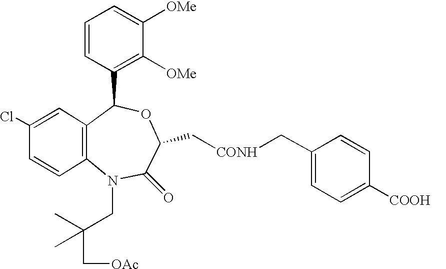

To a mixture of 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethylbenzoic acid obtained in Example 75-(2) (0.2 g, 0.328 mmol), pyridine (0.12 g, 1.48 mmol) and ethyl acetate (2 ml) was added acetyl chloride (90 mg, 1.15 mmol). The mixture was stirred at room temperature for 1 hour and, after addition of water (2 ml), it was further stirred at room temperature for 2 hours. The organic layer was separated, washed with 1 N hydrochloric acid and saturated saline, dried by sodium sulfate and concentrated under reduced pressure to obtain 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethylbenzoic acid (0.15 g, 0.230 mmol, 70%) as a colorless amorphous powder.

[α]_{D}²²−204.2° (c=0.43, MeOH).

IR νₘₐₓ (KBr) cm⁻¹: 3600-2400 (br, COOH, OH), 1716, 1674 (C═O).

¹H-NMR (CDCl₃) δ: 0.945 (3H, s), 1.009 (3H, s), 2.035 (3H, s), 2.748 (1H, dd, J=5.4, 14.2 Hz), 2.945 (1H, dd, J=7.6, 14.2 Hz), 3.539 (1H, d, J=13.8 Hz), 3.601 (3H, s), 3.717 (1H, d, J=11.0 Hz), 3.873 (1H, d, J=11.0 Hz), 3.892 (3H, s), 4.41-4.58 (4H, m), 6.253 (1H, s), 6.539 (1H, br), 6.644 (1H, d, J=2.0 Hz), 6.96-7.36 (7H, m), 7.967 (2H, d, J=8.6 Hz).

Elemental analysis (C₃₄H₃₇N₂O₀Cl.0.2H₂O) Cal'd: C, 62.18; H, 5.74; N, 4.27. Found: C, 62.06; H, 5.88; N, 4.09.

### EXAMPLE 77

α-[4-[2-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]isobutanoic acid

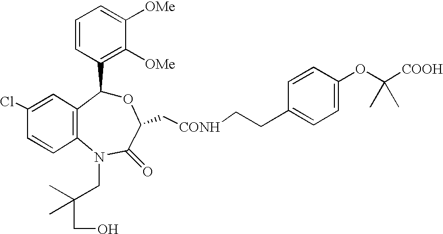

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (1 g, 2.09 mmol) and ethyl-[4-(2-aminoethyl)phenyloxy]isobutanoate (0.58 g, 2.30 mmol) in N,N-dimethylformamide (10 ml) were added diethyl cyanophosphate (0.41 g, 2.51 mmol) and then triethylamine (0.32 g, 3.14 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by recrystallization from ethyl acetate-hexane (1:1) to obtain ethyl α-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]isobutnoate (1.19 g, 1.67 mmol, 80%) as a colorless powder.

Melting point 147-148° C.

[α]_{D}²²−154.9° (c=0.16, MeOH).

IR νₘₐₓ (KBr) cm⁻¹: 3600-3200 (br, NH, OH), 1732, 1653 (C═O).

¹H-NMR (CDCl₃) δ: 0.636 (3H, s), 1.044 (3H, s), 1.255 (3H, t, J=7.0 Hz), 1.577 (6H, s), 2.588 (1H, dd, J=6.2, 14.6 Hz), 2.731 (2H, t, J=7.0 Hz), 2.810 (1H, dd, J=8.0, 14.6 Hz), 3.08-3.50 (5H, m), 3.605 (3H, s), 3.890 (3H, s), 4.239 (2H, q, J=7.0 Hz), 4.37-4.47 (2H, m), 5.80 (1H br), 6.143 (1H, s), 6.603 (1H, s), 6.76-7.35 (9H, m).

Elemental analysis (C₃₈H₄₇N₂O₉Cl.0.2H₂O) Cal'd: C, 63.85; H, 6.68; N, 3.92. Found: C, 63.75; H, 6.45; N, 3.72.

(2) A mixture of ethyl α-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]isobutanoate (1.0 g, 1.41 mmol), 1 N aqueous sodium hydroxide solution (3 ml) and ethanol (25 ml) was stirred at 60° C. for 1 hour. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (11) to obtain α-[4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]isobutanoic acid (0.65 g, 0.951 mmol, 67%) as a colorless powder.

Melting point 209-211° C. (AcOEt-hexane).

[α]_{D}²²−152.2° (c=0.19, MeOH).

IR νₘₐₓ (KBr) cm⁻¹: 3600-3200 (br, COOH, OH), 1732, 1653 (C═O).

¹H-NMR (CDCl₃) δ: 0.626 (3H, s), 1.055 (3H, s), 1.588 (3H, s), 1.599 (3H, s), 2.562 (1H, dd, J=4.8, 14.0 Hz), 2.68-2.85 (3H, m), 3.170 (1H, d, J=12.4 Hz), 3.353 (1H, d, J=14.2 Hz), 3.42-3.52 (2H, m), 3.566 (1H, d, J=12.4 Hz), 3.579 (3H, s), 3.883 (3H, s), 4.30-4.37 (2H, m), 5.916 (1H br), 6.073 (1H, s), 6.597 (1H, s), 6.85-7.34 (9H, m).

Elemental analysis (C₃₆H₄₃N₂O₉Cl) Cal'd: C, 63.29; H, 6.34; N, 4.10. Found: C, 63.07; H, 6.29; N, 3.87.

### EXAMPLE 78

α-[4-[2-[[(3R,5S)-1-(3-Acetoxy-2,2-dimethylpropyl)-7-chloro-5-(2,3-dimethoxyphenyl)-1,2,3,5-tetrahydro-2-oxo-4,1-benzoxazepin-3-yl]acetyl]aminoethyl]phenyloxy]isobutanoic acid

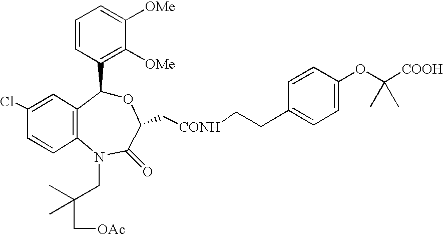

To a mixture of α-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]isobutanoic acid obtained in Example 77-(2) (0.15 g, 0.220 mmol), pyridine (78 mg, 0.99 mmol) and ethyl acetate (5 ml) was added acetyl chloride (60 mg, 0.77 mmol). After stirring at room temperature for 1 hour, water (4 ml) was added to this mixture, followed by stirring at room temperature for additional 2 hours. The organic layer was separated and washed with 1 N hydrochloric acid and saturated saline. This was dried with sodium sulfate and concentrated under reduced pressure. The residue was purified by silica gel column chromatography [eluent:ethyl acetate-methanol (10:1)] to obtain α-[4-[2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminoethyl]phenyloxy]isobutanoic acid (0.11 g, 0.152 mmol, 69%) as a colorless amorphous powder.

[α]_{D}²²−142.3° (c=0.19, MeOH).

IR νₘₐₓ (KBr) cm⁻¹: 3600-3200 (br, COOH), 1736, 1676 (C═O).

¹H-NMR (CDCl₃) δ: 0.936 (3H, s), 1.572 (6H, s), 2.026 (3H, s), 2.592 (1H, dd, J=6.0, 14.0 Hz), 2.78-2.82 (3H, m), 3.40-3.55 (3H, m), 3.597 (3H, s), 3.734 (1H, d, J=10.6 Hz), 3.862 (1H, d, J=10.6 Hz), 3.889 (3H, s), 4.34-4.40 (1H, m), 4.496 (1H, d, J=14.2 Hz), 6.00-6.10 (1H, br), 6.231 (1H, s), 6.632 (1H, s), 6.81-7.33 (9H, m).

Elemental analysis (C₃₈H₄₅N₂O₁₀Cl.H₂O) Cal'd: C, 61.41; H, 6.37; N, 3.77. Found: C, 61.57; H, 6.27; N, 3.72.

### EXAMPLE 79

2-[4-[2-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]acetic acid

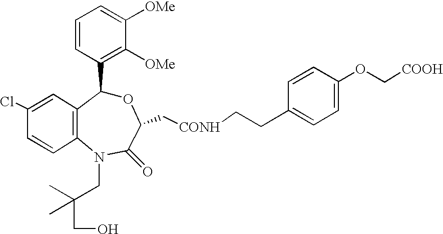

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (1 g, 2.09 mmol) and ethyl 2-[4-(2-aminoethyl)phenyloxy]acetate hydrochloride (0.57 g, 2.20 mmol) in N,N-dimethylformamide (10 ml) were added diethyl cyanophosphate (0.38 g, 2.30 mmol) and then triethylamine (0.53 g, 5.23 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by recrystallization from ethyl acetate-hexane (1:1) to obtain ethyl 2-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]acetate (0.99 g, 1.45 mmol, 69%) as a colorless powder.

Melting point 142-145° C.

[α]_{D}²²−150.9° (c=0.20, MeOH).

IR νₘₐₓ (KBr) cm⁻¹: 3500-3200 (br, NH, OH), 1755, 1653 (C═O).

¹H-NMR (CDCl₃) δ: 0.632 (3H, s), 1.042 (3H, s), 1.302 (3H, t, J=7.0 Hz), 2.590 (1H, dd, J=5.8, 14.6 Hz), 2.72-2.86 (3H, m), 3.06-3.20 (1H, m), 3.33-3.57 (4H, m), 3.601 (3H, s), 3.890 (3H, s), 4.275 (2H, q, J=7.0 Hz), 4.36-4.45 (2H, m), 4.601 (2H, s), 5.813 (1H br), 6.138 (1H, s), 6.610 (1H, s), 6.82-7.35 (9H, m).

Elemental analysis (C₃₆H₄₃N₂O₉Cl.H₂O) Cal'd: C, 61.66; H, 6.47; N, 4.00. Found: C, 61.88; H, 6.21; N, 4.06.

(2) A mixture of ethyl 2-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]acetate obtained in Example 79-(1) (0.89 g, 1.30 mmol), 1 N aqueous sodium hydroxide solution (3 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by column chromatography [eluent:ethyl acetate-methanol (2:1)] to obtain 2-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]acetic acid (0.52 g, 0.794 mmol, 61%) as a colorless amorphous powder.

[α]_{D}²²−160.3° (c=0.22, MeOH).

IR νₘₐₓ (KBr) cm⁻¹: 3600-3200 (br, COOH, OH), 1739, 1651 (C═O).

¹H-NMR (CDCl₃) δ: 0.625 (3H, s), 1.039 (3H, s), 2.572 (1H, dd, J=5.2, 14.0 Hz), 2.69-2.85 (3H, m), 3.169 (1H, d, J=11.6 Hz), 3.353 (1H, d, J=15.0 Hz), 3.42-3.61 (3H, m), 3.581 (3H, s), 3.885 (3H, s), 4.32-4.44 (2H, m), 4.623 (2H, s), 5.920 (1H br), 6.087 (1H, s), 6.609 (1H, s), 6.74-7.38 (9H, m).

Elemental analysis (C₃₄H₃₉N₂O₉Cl.0.5H₂O) Cal'd: C, 61.49; H, 6.07; N, 4.22. Found: C, 61.22; H, 6.35; N, 4.04.

### EXAMPLE 80

2-[4-[2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminoethyl]phenyloxy]acetic acid

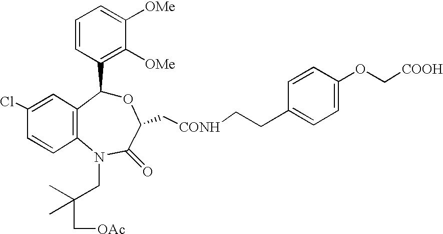

To a mixture of 2-[4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminoethyl]phenyloxy]acetic acid obtained in Example 79-(2) (0.3 g, 0.458 mmol), pyridine (0.16 g, 2.06 mmol) and ethyl acetate (5 ml) was added acetyl chloride (0.13 g, 1.60 mmol). After stirring at room temperature for 1 hour, water (4 ml) was added to this mixture, followed by stirring at room temperature for additional 2 hours. The organic layer was separated and washed with 1 N hydrochloric acid and saturated saline with saturated saline. This was dried with sodium sulfate and concentrated under reduced pressure. The residue was purified by silica gel column chromatography [eluent:ethyl acetate-methanol (2:1)] to obtain 2-[4-[2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminoethyl]phenyloxy]acetic acid (0.12 g, 0.165 mmol, 36%) as a colorless amorphous powder.

[α]_{D}²²−153.8° (c=0.18, MeOH).

IR νₘₐₓ (KBr) cm⁻¹: 3600-3200 (br, COOH), 1732, 1674 (C═O).

¹H-NMR (CDCl₃) δ: 0.932 (3H, s), 0.989 (3H, s), 2.020 (3H, s), 2.593 (1H, dd, J=6.0, 15.2 Hz), 2.68-2.84 (3H, m), 3.38-3.55 (3H, m), 3.594 (3H, s), 3,722 (1H, d, J=11.0 Hz), 3.858 (1H, d, J=11.0 Hz), 3.883 (3H, s), 4.33-4.40 (1H, m), 4.501 (1H, d, J=13.8 Hz), 4.586 (2H, s), 6.103 (1H br), 6.228 (1H, s), 6.623 (1H, s), 6.79-7.37 (9H, m).

Elemental analysis (C₃₆H₄₁N₂O₁₀Cl.0.5H₂O) Cal'd: C, 61.23; H, 5.99; N, 3.97. Found: C, 61.22; H, 6.13; N, 3.94.

### EXAMPLE 81

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]benzofuran-2-carboxylic acid

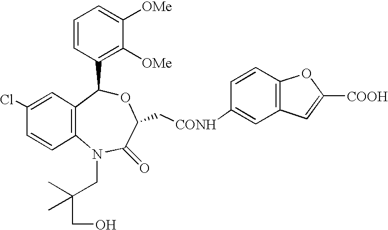

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 5-aminobenzofuran-2-carboxylate hydrochloride (0.48 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel chromatography [eluent:ethyl acetate-hexane (1:1)] to obtain methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]benzofuran-2-carboxylate (1.1 g, 1.59 mmol, 83%) as a colorless amorphous powder.

[α]_{D}²²−95.7° (c=0.15, MeOH).

IR νₘₐₓ (KBr) cm⁻¹: 3331 (NH), 1734, 1678 (C═O).

¹H-NMR (CDCl₃) δ: 0.961 (3H, s), 1.022 (3H, s), 2.011 (3H, s), 2.864 (1H, dd, J=5.8, 14.4 Hz), 3.040 (1H, dd, J=7.2, 14.4 Hz), 3.543 (1H, d, J=14.4 Hz), 3.617 (3H, s), 3.738 (1H, d, J=11.4 Hz), 3.877 (1H, d, J=11.4 Hz), 3.894 (3H, s), 3.978 (3H, s), 4.440 (1H, dd, J=5.8, 7.2 Hz), 4,567 (1H, d, J=14.4 Hz), 6.313 (1H, s), 6.648 (1H, d, J=1.8 Hz), 6.96-7.51 (8H, m), 8.063 (1H, d, J=2.2 Hz), 8.07-8.14 (1H, br).

Elemental analysis (C₃₆H₃₇N₂O₁₀Cl) Cal'd: C, 62.38; H, 5.38; N, 4.04. Found: C, 62.19; H, 5.59; N, 3.80.

(2) A mixture of methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]benzofuran-2-carboxylate obtained in Example 81-(1) (1 g, 1.44 mmol), 1 N aqueous sodium hydroxide solution (3 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethanol-hexane (1:2) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]benzofuran-2-carboxylic acid (0.72 g, 1.13 mmol, 78%) as colorless prisms.

Melting point 171-172° C.

[α]_{D}²²−108.5° (c=0.16, MeOH).

IR νₘₐₓ (KBr) cm⁻¹: 3600-2400 (br, COOH, NH, OH), 1714, 1658 (C═O).

¹H-NMR (CDCl₃) δ: 0.670 (3H, s), 1.059 (3H, s), 2.906 (1H, dd, J=5.6, 14.4 Hz), 3.094 (1H, dd, J=7.8, 14.4 Hz), 3.228 (1H, d, J=12.0 Hz), 3.418 (1H, d, J=14.0 Hz), 3.610 (3H, s), 3.648 (1H, d, J=12.0 Hz), 3.888 (3H, s), 4.47-4.53 (2H, m), 6.204 (1H, s), 6.627 (1H, s), 6.97-7.46 (8H, m), 7.984 (1H, s), 8.16-8.28 (1H, br).

Elemental analysis (C₃₃H₃₃N₂O₉Cl.H₂O) Cal'd: C, 60.50; H, 5.38; N, 4.28. Found: C, 60.43; H, 5.40; N, 4.10.

### EXAMPLE 82

5-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]benzofuran-2-carboxylic acid

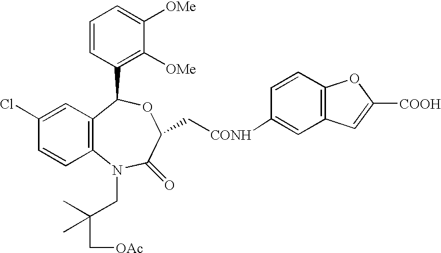

To a mixture of 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]benzofuran-2-carboxylate acid obtained in Example 81-(2) (0.3 g, 0.471 mmol), pyridine (0.17 g, 2.12 mmol) and ethyl acetate (5 ml) was added acetyl chloride (0.13 g, 1.65 mmol). After stirring at room temperature for 1 hour, water (4 ml) was added to this mixture, followed by stirring at room temperature for additional 2 hours. The organic layer was separated and washed with 1 N hydrochloric acid and saturated saline. This was dried with sodium sulfate and concentrated under reduced pressure to obtain 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]benzofuran-2-carboxylic acid (0.28 g, 0.412 mmol, 88%) as a colorless amorphous powder.

[α]_{D}²²−95.3° (c=0.21, MeOH).

IR νₘₐₓ (KBr) cm⁻¹: 3600-2400 (br, COOH, NH), 1732, 1682 (C═O).

¹H-NMR (CDCl₃) δ: 0.976 (3H, s), 1.033 (3H, s), 1.941 (3H, s), 2.918 (1H, dd, J=5.2, 15.4 Hz), 3.227 (1H, dd, J=8.8, 15.4 Hz), 3.610 (3H, s), 3.614 (1H, d, J=14.6 Hz), 3.806 (1H, d, J=11.0 Hz), 3.883 (3H, s), 3.885 (1H, d, J=11.0 Hz), 4.56-4.65 (2H, m), 6.346 (1H, s), 6.672 (1H, d, J=1.8 Hz), 6.95-7.45 (8H, m), 7.921 (1H, s), 8.84-8.96 (1H, br).

Elemental analysis (C₃₅H₃₅N₂O₁₀Cl.0.5H₂O) Cal'd: C, 61.09; H, 5.27; N, 4.07. Found: C, 61.00; H, 5.26; N, 3.85.

### EXAMPLE 83

7-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]indole-2-carboxylic acid

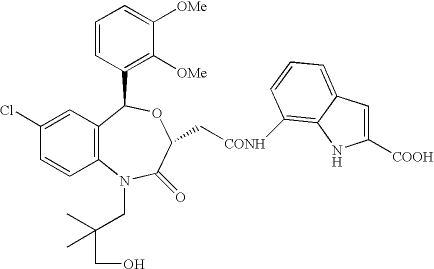

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.03 mmol) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 7-aminoindole-2-carboxylate hydrochloride obtained in Example 49-(3) (0.51 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (1:1)] to obtain ethyl 7-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]indole-2-carboxylate (1.1 g, 1.56 mmol, 81%) as a colorless amorphous powder.

[α]_{D}²²−115.3° (c=0.22, MeOH).

IR νₘₐₓ (KBr) cm⁻¹: 3296 (NH), 1712, 1666 (C═O).

¹H-NMR (CDCl₃) δ: 0.986 (3H, s), 1.046 (3H, s), 1.394 (3H, t, J=7.4 Hz), 2.011 (3H, s), 2.931 (1H, dd, J=5.2, 13.6 Hz), 3.119 (1H, dd, J=8.2, 13.6 Hz), 3.543 (1H, d, J=14.4 Hz), 3.621 (3H, s), 3.781 (1H, d, J=11.0 Hz), 3.894 (3H, s), 3.929 (1H, d, J=11.0 Hz), 4.387 (2H, q, J=7.4 Hz), 4,473 (1H, dd, J=5.2, 8.2 Hz), 4.718 (1H, d, J=14.4 Hz), 6.328 (1H, s), 6.656 (1H, d, J=2.2 Hz), 6.94-7.54 (10H, m), 8.24-8.28 (1H, br).

(2) A mixture of ethyl 7-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]indole-2-carboxylate (1 g, 1.42 mmol), 1 N aqueous sodium hydroxide solution (3 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by column chromatography [eluent:ethyl acetate-methanol (10:1)] to obtain 7-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]indole-2-carboxylic acid (0.66 g, 1.04 mmol, 73%) as a colorless amorphous powder.

[α]_{D}²²−111.9° (c=0.38, MeOH).

IR νₘₐₓ (KBr) cm⁻¹: 3600-2400 (br, COOH, NH, OH), 1651 (C═O).

¹H-NMR (CD₃OD) δ: 0.828 (3H, s), 0.925 (3H, s), 3.056 (2H, d, J=6.6 Hz), 3.206 (1H, d, J=11.8 Hz), 3.447 (1H, d, J=11.4 Hz), 3.559 (3H, s), 3.616 (1H, d, J=11.8 Hz), 3.859 (3H, s), 4.45-4.52 (2H, m), 6.206 (1H, s), 6.520 (1H, d, J=2.2 Hz), 6.96-7.54 (11H, m).

Elemental analysis (C₃₃H₃₄N₃O₈Cl.1.5H₂O) Cal'd: C, 59.77; H, 5.62; N, 6.34. Found: C, 59.37; H, 5.48; N, 6.43.

### EXAMPLE 84

7-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]indole-2-carboxylic acid

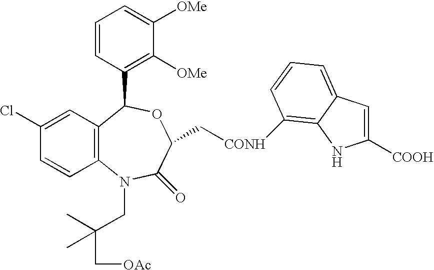

To a mixture of 7-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]indole-2-carboxylate acid obtained in Example 83-(2) (0.3 g, 0.472 mmol), pyridine (0.17 g, 2.12 mmol) and ethyl acetate (5 ml) was added acetyl chloride (0.13 g, 1.65 mmol). After stirring at room temperature for 1 hour, water (4 ml) was added to this mixture, followed by stirring at room temperature for additional 2 hours. The organic layer was separated and washed with 1 N hydrochloric acid and saturated saline. This was dried with sodium sulfate and concentrated under reduced pressure to obtain 7-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]indole-2-carboxylic acid (0.25 g, 0.369 mmol, 78%) as a colorless amorphous powder.

[α]_{D}²²−104.4° (c=0.28, MeOH).

IR νₘₐₓ (KBr) cm⁻¹: 3500-2400 (br, COOH, NH), 1682 (C═O).

¹H-NMR (CD₃OD) δ: 1.020 (3H, s), 1.038 (3H, s), 2.024 (3H, s), 3.046 (2H, d, J-6.6 Hz), 3.608 (3H, s), 3.641 (1H, d, J=14.2 Hz), 3.770 (1H, d, J=9.4 Hz), 3.870 (1H, d, J=9.4 Hz), 3.889 (3H, s), 4.528 (1H, t, J=6.6 Hz), 4.61 (1H, t, J=14.2 Hz), 6.321 (1H, s), 6.581 (1H, d, J=2.6 Hz), 7.02-7.58 (11H, m).

Elemental analysis (C₃₅H₃₆N₃O₉Cl.0.5H₂O) Cal'd: C, 61.18; H, 5.43; N, 6.12. Found: C, 61.42; H, 5.83; N, 6.46.

### EXAMPLE 85

4-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid

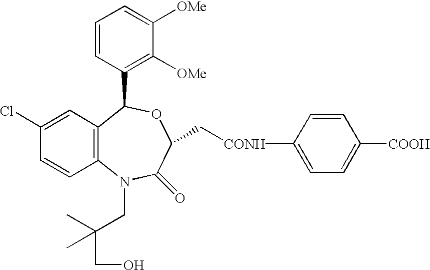

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.03 ml) in tetrahydrofuran (10 ml) was added thionyl chloride (0.67 g, 5.61 mmol) at room temperature. After stirring for 1 hour, the mixture was concentrated under reduced pressure. The residue was dissolved in tetrahydrofuran (5 ml) and added to a mixture of ethyl 4-aminobenzoate (0.51 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (3:2)] to obtain ethyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate (1.01 g, 1.51 mmol, 79%) as a colorless amorphous powder.

[α]_{D}²²−116.1° (c=0.18, MeOH).

IR νₘₐₓ (KBr) cm⁻: 3331 (NH), 1716, 1682 (C═O).

¹H-NMR (CDCl₃) δ: 0.958 (3H, s), 1.015 (3H, s), 1.258 (3H, t, J=7.4 Hz), 2.029 (3H, s), 2.858 (1H, dd, J=5.8, 14.2 Hz), 3.016 (1H, dd, J=7.4, 14.2 Hz), 3.540 (1H, d, J=14.4 Hz), 3.616 (3H, s), 3.732 (1H, d, J=11.0 Hz), 3.876 (1H, d, J=11.0 Hz), 3.892 (3H, s), 4.30-4.44 (3H, m), 4.561 (1H, d, J=14.4 Hz), 6.303 (1H, s), 6.649 (1H, d, J=1.8 Hz), 6.96-7.39 (5H, m), 7.564 (2H, d, J=8.4 Hz), 7.983 (2H, d, J=8.4 Hz), 8.210 (1H, br).

Elemental analysis (C₃₅H₃₉N₂O₉Cl) Cal'd: C, 63.01; H, 5.89; N, 4.20. Found: C, 62.74; H, 5.91; N, 4.13.

(2) A mixture of ethyl 4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate obtained in Example 85-(1) (0.9 g, 1.35 mmol), 1 N aqueous sodium hydroxide solution (3 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by column chromatography [eluent: ethyl acetate-methanol (10:1)] to obtain 4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid (0.17 g, 0.285 mmol, 21%) as a colorless amorphous powder.

[α]_{D}²²−112.8° (c=0.18, MeOH).

IR νₘₐₓ (KBr) cm⁻¹: 3600-2400 (br, COOH, OH), 1682, 1653 (C═O).

¹H-NMR (CD₃OD) δ: 0.665 (3H, s), 1.059 (3H, s), 2.889 (1H, dd, J=5.4, 13.4 Hz), 3.046 (1H, dd, J=6.6, 13.4 Hz), 3.187 (1H, d, J=12.4 Hz), 3.408 (1H, d, J=14.4 Hz), 3.614 (3H, s), 3.625 (1H, d, J=12.4 Hz), 3.408 (1H, d, J=14.4 Hz), 3.614 (3H, s), 3.625 (1H, d, J=12.4 Hz), 3.896 (3H, s), 4.42-4.53 (2H, m), 6.208 (1H, s), 6.640 (1H, d, J=2.0 Hz), 6.97-7.37 (5H, m), 7.607 (2H, d, J=8.8 Hz), 8.051 (2H, d, J=8.8 Hz), 8.12-8.24 (1H, br).

Elemental analysis (C₃₁H₃₃N₂O₈Cl.0.5H₂O) Cal'd: C, 61.44; H, 5.65; N, 4.62. Found: C, 61.64; H, 5.73; N, 4.60.

### EXAMPLE 86

3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid

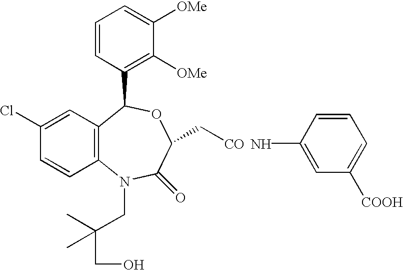

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (0.5 g, 0.962 mmol) and N,N-dimethylformamide (0.02 ml) in tetrahydrofuran (5 ml) was added thionyl chloride (0.34 g, 2.81 mmol) at room temperature. After stirring for 1 hour, the mixture was concentrated under reduced pressure. The residue was dissolved in tetrahydrofuran (5 ml) and added to a mixture of methyl 3-aminobenzoate (0.16 g, 1.06 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (4:3)] to obtain methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate (0.47 g, 0.720 mmol, 75%) as a colorless amorphous powder.

[α]_{D}²²−135.4° (c=0.16, MeOH).

IR νₘₐₓ (KBr) cm⁻¹: 3331 (NH), 1724, 1682 (C═O).

¹H-NMR (CDCl₃) δ: 0.962 (3H, s), 1.024 (3H, s), 2.024 (3H, s), 2.853 (1H, dd, J=6.4, 14.0 Hz), 3.011 (1H, dd, J=7.2, 14.0 Hz), 3.542 (1H, d, J=13.4 Hz), 3.623 (3H, s), 3.734 (1H, d, J=11.4 Hz), 3.879 (1H, d, J=11.4 Hz), 3.896 (3H, s), 3.961 (3H, s), 4.420 (1H, dd, J=6.4, 7.2 Hz), 4.572 (1H, d, J=13.4 Hz), 6.310 (1H, s), 6.655 (1H, d, J=1.8 Hz), 6.97-7.42 (5H, m), 7.76-7.86 (2H, m), 8.02-8.12 (2H, m).

Elemental analysis (C₃₄H₃₇N₂O₉Cl) Cal'd: C, 62.53; H, 5.71; N, 4.29. Found: C, 62.37; H, 5.72; N, 4.15.

(2) A mixture of methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate obtained in Example 86-(1) (0.37 g, 0.567 mmol), 1 N aqueous sodium hydroxide solution (2 ml) and ethanol (4 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid (0.33 g, 0.553 mmol, 97%) as colorless prisms.

[α]_{D}²²−149.8° (c=0.37, MeOH).

IR νₘₐₓ (KBr) cm⁻¹: 3427, 3358 (NH, OH), 3600-2400 (br, COOH), 1697, 1651 (C═O).

¹H-NMR (CDCl₃) δ: 0.665 (3H, s), 1.053 (3H, s), 2.887 (1H, dd, J=5.4, 14.4 Hz), 3.059 (1H, dd, J=7.2, 14.4 Hz), 3.200 (1H, d, J=11.8 Hz), 3.400 (1H, d, J=13.6 Hz), 3.618 (3H, s), 3.636 (1H, d, J=11.8 Hz), 3.888 (3H, s), 4.44-4.53 (2H, m), 6.203 (1H, s), 6.627 (1H, s), 6.96-7.45 (6H, m), 7.823 (1H, d, J=8.2 Hz), 7.962 (2H, d, J=8.2 Hz), 8.068 (1H, s), 8.16-8.30 (1H, br).

Elemental analysis (C₃₁H₃₃N₂O₈Cl.H₂O) Cal'd: C, 60.53; H, 5.74; N, 4.55. Found: C, 60.69; H, 5.72; N, 4.50.

### EXAMPLE 87

3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoic acid

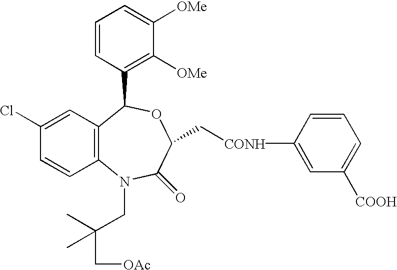

To a mixture of 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid obtained in Example 86-(2) (0.1 g, 0.167 mmol), pyridine (60 mg, 0.752 mmol) and ethyl acetate (3 ml) was added acetyl chloride (46 mg, 0.585 mmol). After stirring at room temperature for 1 hour, water (4 ml) was added to this mixture, followed by stirring at room temperature for additional 1 hours. The organic layer was separated and washed with 1 N hydrochloric acid and saturated saline. This was dried with sodium sulfate and concentrated under reduced pressure to obtain 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoic acid (94 mg, 0.147 mmol, 88%) as a colorless amorphous powder.

[α]_{D}²²−142.1° (c=0.27, MeOH).

IR νₘₐₓ (KBr) cm⁻: 3600-2400 (br, COOH, NH), 1722, 1680 (C═O).

¹H-NMR (CDCl₃) δ: 0.963 (3H, s), 1.018 (3H, s), 2.022 (3H, s), 2.892 (1H, dd, J=6.0, 13.6 Hz), 3.098 (1H, dd, J=7.6, 13.6 Hz), 3.540 (1H, d, J=14.0 Hz), 3.621 (3H, s), 3.753 (1H, d, J=11.0 Hz), 3.887 (1H, d, J=11.0 Hz), 3.888 (3H, s), 4.481 (1H, dd, J=6.0, 7.6 Hz), 4.575 (1H, d, J=14.0 Hz), 6.306 (1H, s), 6.660 (1H, d, J=1.4 Hz), 7.791 (1H, d, J=8.0 Hz), 8.02-8.05 (2H, m), 8.48-8.58 (1H, br).

Elemental analysis (C₃₃H₃₅N₂O₉Cl.0.5H₂O) Cal'd: C, 61.16; H, 5.60; N, 4.32. Found: C, 61.28; H, 5.32; N, 4.46.

### EXAMPLE 88

2-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid

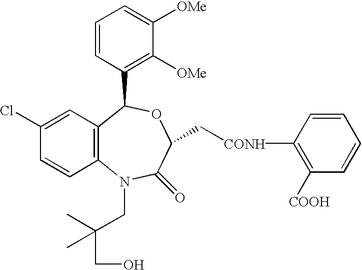

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (0.5 g, 0.962 mmol) and N,N-dimethylformamide (0.02 ml) in tetrahydrofuran (5 ml) was added thionyl chloride (0.34 g, 2,81 mmol) at room temperature. After stirring for 1 hour, the mixture was concentrated under reduced pressure. The residue was dissolved in tetrahydrofuran (5 ml) and added to a mixture of methyl 2-aminobenzoate (0.16 g, 1.06 mmol), triethylamine (0.24 g, 2.41 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (2:1)] to obtain methyl 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate (0.28 g, 0.429 mmol, 45%) as a colorless amorphous powder.

[α]_{D}²²−175.0° (c=0.25, MeOH).

IR νₘₐₓ (KBr) cm⁻¹: 3275 (NH), 1738, 1682 (C═O).

¹H-NMR (CDCl₃) δ: 0.953 (3H, s), 1.030 (3H, s), 2.030 (3H, s), 2.946 (1H, dd, J=6.2, 15.0 Hz), 3.118 (1H, dd, J=6.6, 15.0 Hz), 3.551 (1H, d, J=14.2 Hz), 3.614 (3H, s), 3.735 (1H, d, J=11.0 Hz), 3.856 (1H, d, J=11.0 Hz), 3.861 (3H, s), 3.883 (3H, s), 4.509 (1H, dd, J=6.2, 6.6 Hz), 4.588 (1H, d, J=14.2 Hz), 6.299 (1H, s), 6.630 (1H, s), 6.93-7.55 (7H, m), 8.019 (1H, dd, J=2.0, 8.2 Hz), 8.631 (1H, d, J=8.4 Hz).

Elemental analysis (C₃₄H₃₇N₂O₉Cl) Cal'd: C, 62.53; H, 5.71; N, 4.29. Found: C, 62.69; H, 5.57; N, 4.08.

(2) A mixture of methyl 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoate obtained in Example 88-(1) (0.23 g, 0.352 mmol), 1 N aqueous sodium hydroxide solution (1.2 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid (0.18 g, 0.301 mmol, 86%) as a colorless amorphous powder.

[α]_{D}²²−181.2° (c=0.11, MeOH).

IR νₘₐₓ (KBr) cm⁻¹: 3600-2400 (br, COOH, NH, OH), 1682, 1657 (C═O).

¹H-NMR (CDCl₃) δ: 0.661 (3H, s), 1.060 (3H, s), 2.960 (1H, dd, J=5.8, 14.6 Hz), 3.169 (1H, dd, J=7.2, 14.6 Hz), 3.222 (1H, d, J=12.4 Hz), 3.402 (1H, d, J=14.4 Hz), 3.603 (3H, s), 3.686 (1H, d, J=12.4 Hz), 3.854 (3H, s), 4.488 (1H, d, J=14.4 Hz), 4.529 (1H, dd, J=5.8, 7.2 Hz), 6.176 (1H, s), 6.616 (1H, s), 6.93-7.56 (7H, m), 8.078 (1H, d, J=8.4 Hz), 8.613 (1H, d, J=8.4 Hz).

Elemental analysis (C₃₁H₃₃N₂O₈Cl.0.5H₂O) Cal'd: C, 61.44; H, 5.65; N, 4.62. Found: C, 61.65; H, 5.49; N, 4.63.

### EXAMPLE 89

2-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoic acid

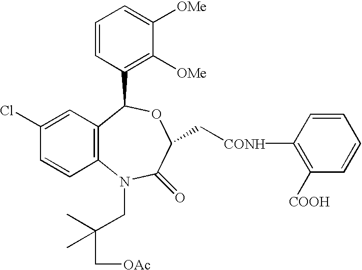

To a mixture of 2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminobenzoic acid obtained in Example 88-(2) (0.1 g, 0.167 mmol), pyridine (60 mg, 0.752 mmol) and ethyl acetate (3 ml) was added acetyl chloride (46 mg, 0.585 mmol). After stirring at room temperature for 1 hour, water (3 ml) was added to this mixture, followed by stirring at room temperature for additional 1 hours. The organic layer was separated and washed with 1 N hydrochloric acid and saturated saline. This was dried with sodium sulfate and concentrated under reduced pressure to obtain 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminobenzoic acid (73 mg, 0.114 mmol, 68%) as a colorless amorphous powder.

[α]_{D}²²−154.7° (c=0.29, MeOH).

IR νₘₐₓ (KBr) cm⁻¹: 3600-2400 (br, COOH, NH), 1738, 1682 (C═O).

¹H-NMR (CDCl₃) δ: 0.961 (3H, s), 1.023 (3H, s), 2.031 (3H, s), 2.980 (1H, dd, J=5.4, 15.0 Hz), 3.259 (1H, dd, J=6.2, 15.0 Hz), 3.567 (1H, d, J=13.6 Hz), 3.614 (3H, s), 3.771 (1H, d, J=11.0 Hz), 3.860 (3H, s), 3.876 (3H, d, J=11.0 Hz), 4.559 (1H, dd, J=5.4, 6.2 Hz), 4.609 (1H, d, J=13.6 Hz), 6.309 (1H, s), 6.646 (1H, s), 6.92-7.56 (7H, m), 8.039 (1H, dd, J=1.4, 8.0 Hz), 8.639 (1H, d, J=8.0 Hz).

Elemental analysis (C₃₃H₃₅N₂O₉Cl) Cal'd: C, 62.02; H, 5.52; N, 4.38. Found: C, 61.88; H, 5.82; N, 4.20.

### EXAMPLE 90

3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-thiophenecarboxylic acid

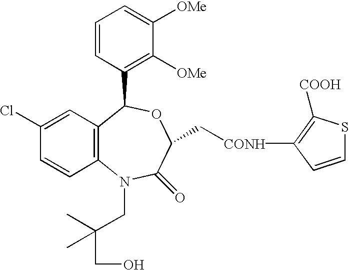

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 3-amino-2-thiophenecarboxylate (0.33 g, 2.11 mmol), triethylamine (0.29 g, 2.88 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (1: 2)] to obtain methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-thiophenecarboxylate (0.58 g, 0.880 mmol, 46%) as a colorless amorphous powder.

[α]_{D}²²−202.0° (c=0.12, MeOH).

IR νₘₐₓ (KBr) cm⁻¹: 3325 (NH), 1734, 1680 (C═O).

¹H-NMR (CDCl₃) δ: 0.949 (3H, s), 1.026 (3H, s), 2.026 (3H, s), 2.923 (1H, dd, J=6.0, 15.2 Hz), 3.097 (1H, dd, J=6.6, 15.2 Hz), 3.548 (1H, d, J=14.0 Hz), 3.618 (3H, s), 3.70-3.75 (2H, m), 3.836 (3H, s), 3.885 (3H, s), 4.473 (1H, dd, J=6.0, 6.6 Hz), 4.583 (1H, d, J=14.0 Hz), 6.299 (1H, s), 6.638 (1H, s), 6.95-7.33 (3H, m), 7.436 (1H, d, J=5.4 Hz), 8.062 (1H, d, J=5.4 Hz).

Elemental analysis (C₃₂H₃₅N₂₀₉SCl) Cal'd: C, 58.31; H, 5.35; N, 4.25. Found: C, 58.29; H, 5.34; N, 4.24.

(2) A mixture of methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-thiophenecarboxylate obtained in Example 86-(1) (0.5 g, 0.759 mmol), 1 N aqueous sodium hydroxide solution (1.5 ml) and ethanol (5 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-thiophenecarboxylic acid (0.30 g, 0.497 mmol, 66%) as colorless prisms.

Melting point 154-155°.

[α]_{D}²²−193.1° (c=0.15, MeOH).

IR νₘₐₓ (KBr) cm⁻¹: 3600-2400 (br, COOH, NH, OH), 1697, 1680, 1666 (C═O).

¹H-NMR (CDCl₃) δ: 0.665 (3H, s), 1.055 (3H, s), 2.957 (1H, dd, J=5.8, 15.0 Hz), 3.147 (1H, dd, J=7.0, 15.0 Hz), 3.218 (1H, d, J=11.6 Hz), 3.401 (1H, d, J=14.0 Hz), 3.612 (3H, s), 3.661 (1H, d, J=11.6 Hz), 3.848 (3H, s), 4.45-4.52 (2H, m), 6.176 (1H, s), 6.614 (1H, s), 6.93-7.36 (5H, m), 7.498 (1H, d, J=5.4 Hz), 8.067 (2H, d, J=5.4 Hz).

Elemental analysis (C₂₉H₃₁N₂O₈SC-Et₂O) Cal'd: C, 58.53; H, 6.10; N, 4.13. Found: C, 58.42; H, 5.74; N, 4.25.

### EXAMPLE 91

3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-thophenecarboxylic acid

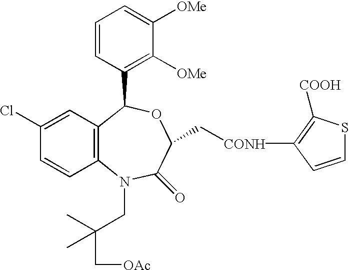

To a mixture of 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-thiophenecarboxylic acid obtained in Example 90-(2) (0.15 g, 0.249 mmol), pyridine (88 mg, 1.12 mmol) and ethyl acetate (3 ml) was added acetyl chloride (68 mg, 0.871 mmol). After stirring at room temperature for 1 hour, water (3 ml) was added to this mixture, followed by stirring at room temperature for additional 2 hours. The organic layer was separated and washed with 1 N hydrochloric acid and saturated saline. This was dried with sodium sulfate and concentrated under reduced pressure to obtain 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-thiophenecarboxylic acid (0.12 g, 0.184 mmol, 76%) as a colorless amorphous powder.

[α]_{D}²²−188.4° (c=0.23, MeOH).

IR νₘₐₓ (KBr) cm⁻¹: 3600-2400 (br, COOH, NH), 1736, 1678 (C═O).

¹H-NMR (CDCl₃) δ: 0.956 (3H, s), 1.018 (3H, s), 2.033 (3H, s), 2.984 (1H, dd, J=6.2, 15.6 Hz), 3.263 (1H, dd, J=6.8, 15.6 Hz), 3.562 (1H, d, J=14.0 Hz), 3.622 (3H, s), 3.750 (1H, d, J=11.2 Hz), 3.854 (3H, s), 3.866 (1H, d, J=11.2 Hz), 4.517 (1H, dd, J=6.2, 6.8 Hz), 4.604 (1H, d, J=14.0 Hz), 6.298 (1H, s), 6.647 (1H, s), 6.93-7.36 (5H, m), 7.482 (1H, d, J=5.6 Hz), 8.081 (1H, d, J=5.6 Hz).

Elemental analysis (C₃₁H₃₃N₂O₉SCl) Cal'd: C, 57.72; H, 5.16; N, 4.34. Found: C, 57.66; H, 5.21; N, 4.31.

### EXAMPLE 92

2-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminothiazole-4-acetic acid

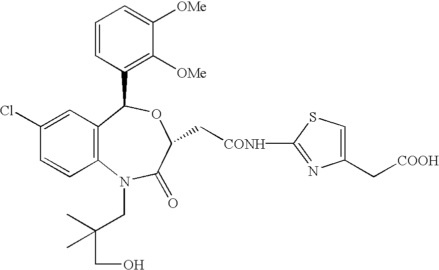

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 2-aminothiazole-4-acetate hydrochloride (0.44 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (1:1)] to obtain methyl 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminothiazole-4-acetate (0.54 g, 0.801 mmol, 42%) as a colorless amorphous powder.

[α]_{D}²²−140.1° (c=0.13, MeOH).

IR νₘₐₓ (KBr) cm⁻¹: 3261 (NH), 1738, 1680 (C═O).

¹H-NMR (CDCl₃) δ: 0.947 (3H, s), 1.016 (3H, s), 2.024 (3H, s), 2.917 (1H, dd, J=5.8, 14.6 Hz), 3.102 (1H, dd, J=7.0, 14.6 Hz), 3.543 (1H, d, J=14.4 Hz), 3.619 (3H, s), 3.714 (2H, s), 3.725 (3H, s), 3.726 (1H, d, J=11.2 Hz), 3.858 (1H, d, J=11.2 Hz), 3.890 (3H, s), 4.436 (1H, dd, J=5.8, 7.0 Hz), 4.582 (1H, d, J=14.4 Hz), 6.299 (1H, s), 6.655 (1H, d, J=1.4 Hz) 6.775 (1H, s), 6.96-7.35 (5H, m), 9.45-9.60 (1H, br)

Elemental analysis (C₃₂H₃₆N₃O₉SCl) Cal'd: C, 57.01; H, 5.38; N, 6.23. Found: C, 57.13; H, 5.15; N, 6.33.

(2) A mixture of methyl 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminothiazole-4-acetate obtained in Example 92-(1) (0.48 g, 0.712 mmol), 1 N aqueous sodium hydroxide solution (2.2 ml) and ethanol (5 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was washed with ethyl acetate-hexane (1:1) to obtain 2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminothiazole-4-acetic acid (0.33 g, 0.534 mmol, 75%) as a colorless amorphous powder.

[α]_{D}²²−142.6° (c=0.36, MeOH).

IR νₘₐₓ (KBr) cm⁻¹: 3600-2400 (br, COOH, OH, NH), 1660 (C═O).

¹H-NMR (CDCl₃) δ: 0.645 (3H, s), 1.037 (3H, s), 2.941 (1H, dd, J=5.6, 16.0 Hz), 3.13-3.29 (2H, m), 3.391 (1H, d, J=13.6 Hz), 3.605 (3H, s) 3.64-3.70 (3H, m), 3.881 (3H, s), 4.44-4.54 (2H, m), 6.164 (1H, s), 6.614 (1H, s), 6.744 (1H, s), 6.94-7.37 (5H, m).

Elemental analysis (C₂₉H₃₂N₃O₈SCl.0.3H₂O) Cal'd: C, 55.86; H, 5.27; N, 6.74. Found: C, 55.89; H, 5.47; N, 6.57.

### EXAMPLE 93

2-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminothiazole-4-acetic acid

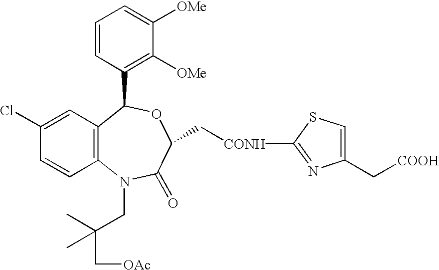

To a mixture of 2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminothiazole-4-acetic acid obtained in Example 92-(2) (0.15 g, 0.243 mmol), pyridine (86 mg, 1.09 mmol) and ethyl acetate (3 ml) was added acetyl chloride (67 mg, 0.849 mmol). After stirring at room temperature for 1 hour, water (4 ml) was added to this mixture, followed by further stirring at room temperature overnight. The organic layer was separated and washed with 1 N hydrochloric acid and saturated saline. This was dried with sodium sulfate and concentrated under reduced pressure to obtain 2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminothiazole-4-acetic acid (0.12 g, 0.182 mmol, 75%) as a colorless amorphous powder.

[α]_{D}²²−122-134.8° (c=0.24, MeOH).

IR νₘₐₓ (KBr) cm⁻¹: 3600-2400 (br, COOH, NH), 1732, 1682 (C═O).

¹H-NMR (CDCl₃) δ: 0.941 (3H, s), 1.015 (3H, s), 2.017 (3H, s), 2.953 (1H, dd, J=4.8, 15.6 Hz), 3.198 (1H, dd, J=7.8, 15.6 Hz), 3.548 (1H, d, J=14.6 Hz), 3.609 (3H, s), 3.664 (2H, s), 3.729 (1H, d, J=11.2 Hz), 3.847 (1H, d, J=11.2 Hz), 3.881 (3H, s), 4.48-4.61 (2H, m), 6.280 (1H, s), 6.649 (1H, s), 6.728 (1H, s), 6.93-7.40 (5H, m).

Elemental analysis (C₃₁H₃₄N₃O₉SCl.H₂O) Cal'd: C, 54.90; H, 5.19; N, 6.21. Found: C, 54.90; H, 5.35; N, 6.21.

### EXAMPLE 94

5-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-furancarboxylic acid

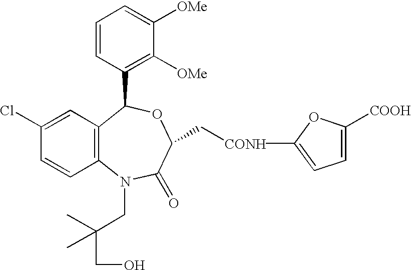

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 5-amino-2-furancarboxylate (0.48 g, 4.80 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel column chromatography [eluent:ethyl acetate-hexane (3:2)] to obtain methyl 5-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-furancarboxylate (0.51 g, 0.793 mmol, 41%) as a colorless amorphous powder.

[α]_{D}²²−178.8° (c=0.13, MeOH).

IR νₘₐₓ (KBr) cm⁻¹: 3281, 3233 (NH), 1728, 1682 (C═O).

¹H-NMR (CDCl₃) δ: 0.954 (3H, s), 1.020 (3H, s), 2.028 (3H, s), 2.903 (1H, dd, J=7.6, 14.6 Hz), 3.012 (1H, dd, J=7.0, 14.6 Hz), 3.547 (1H, d, J=14.4 Hz), 3.630 (3H, s), 3.732 (1H, d, J=11.4 Hz), 3.866 (1H, d, J=11.4 Hz), 3.879 (3H, s), 3.894 (3H, s), 4.385 (1H, dd, J=7.0, 7.6 Hz), 4.589 (1H, d, J=14.4 Hz), 6.312 (1H, s), 6.453 (1H, d, J=3.8 Hz) 6.671 (1H, d, J=2.2 Hz), 6.89-7.35 (6H, m), 8.95-9.00 (1H, br).

Elemental analysis (C₃₂H₃₅N₂O₁₀Cl) Cal'd: C, 59.77; H, 5.49; N, 4.36. Found: C, 59.70; H, 5.41; N, 4.33.

(2) A mixture of methyl 5-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-furancarboxylate obtained in Example 94-(1) (0.41 g, 0.638 mmol), 1 N aqueous sodium hydroxide solution (1.5 ml) and ethanol (5 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue purified by recrystallization from ethyl acetate-hexane (1:1) to obtain 5-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-furancarobxylic acid (0.17 g, 1.14 mmol, 98%) as a colorless powder.

Melting point 155-158° C.

[α]_{D}²²−160.6° (c=0.15, MeOH).

IR νₘₐₓ (KBr) cm⁻¹: 3600-2400 (br, COOH, OH), 1710, 1684, 1655 (C═O).

¹H-NMR (CDCl₃) δ: 0.854 (3H, s), 0.934 (3H, s), 2.848 (1H, dd, J=6.6, 15.4 Hz), 2.996 (1H, dd, =7.0, 15.4 Hz), 3.202 (1H, d, J=11.0 Hz), 3.430 (1H, d, J=11.0 Hz), 3.585 (3H, s) 3.681 (1H, d, J=14.2 Hz), 3.883 (3H, s), 4.428 (1H, d, J=14.2 Hz), 4.460 (1H, dd, J=6.6, 7.0 Hz), 6.200 (1H, s), 6.380 (1H, d, J=3.6 Hz), 6.529 (1H, d, J=2.0 Hz), 7.05-7.63 (6H, m).

Elemental analysis (C₂₉H₃₁N₂O₉Cl.1.5H₂O) Cal'd: C, 56.73; H, 5.58; N, 4.56. Found: C, 56.49; H, 5.43; N, 4.28.

### EXAMPLE 95

5-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-furancarboxylic acid

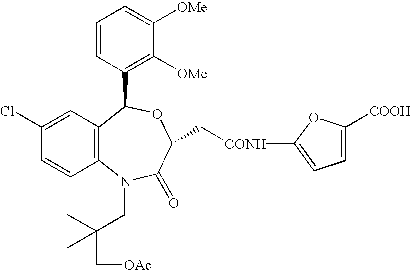

To a mixture of 5-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-2-furancarboxylic acid obtained in Example 94-(2) (0.1 g, 0.170 mmol), pyridine (60 mg, 0.767 mmol) and ethyl acetate (3 ml) was added acetyl chloride (47 mg, 0.596 mmol). After stirring at room temperature for 1 hour, water (3 ml) was added to this mixture, followed by further stirring at room temperature for 3 hours. The organic layer was separated and washed with 1 N hydrochloric acid and saturated saline. This was dried with sodium sulfate and concentrated under reduced pressure to obtain 5-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-2-furancarboxylic acid (83 mg, 0.132 mmol, 78%) as a colorless amorphous powder.

[α]_{D}²²−173.1° (c=0.15, MeOH).

IR νₘₐₓ (KBr) cm⁻¹: 3600-2400 (br, COOH, NH), 1678 (C═O).

¹H-NMR (CDCl₃) δ: 0.998 (6H, s), 2.008 (3H, s), 2.90-2.96 (2H, m), 3.596 (3H, s), 3.725 (1H, d, J=10.6 Hz), 3.733 (1H, d, J=14.0 Hz), 3.830 (1H, d, J=10.6 Hz), 3.885 (3H, s), 4.41-4.53 (2H, m), 6.272 (1H, s), 6.380 (1H, d, J=3.6 Hz), 6.550 (1H, d, J=2.0 Hz), 7.05-7.63 (6H, m).

Elemental analysis (C₃₁H₃₃N₂O₁₀Cl.H₂O) Cal'd: C, 57.54; H, 5.45; N, 4.33. Found: C, 57.63; H, 5.38; N, 4.22.

### EXAMPLE 96

4-[3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]phenylacetic acid

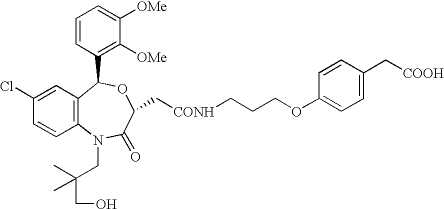

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (1 g, 2.09 mmol) and methyl 4-(3-aminopropyloxy)benzoate hydrochloride (0.57 g, 2.20 mmol) in N,N-dimethylformamide (10 ml) were added diethyl cyanophosphate (0.38 g, 2.30 mmol) and then triethylamine (0.53 g, 5.23 mmol). The mixture was stirred at room temperature for 30 minutes. The mixture was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was recrystallized from hexane-ethyl acetate (1:1) to obtain methyl 4-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoate (1.28 g, 1.87 mmol, 90%) as colorless needles.

Melting point 147-149° C.

[α]_{D}²²−166.8° (c=0.21, MeOH).

IR νₘₐₓ (KBr) cm⁻¹: 3500-3200 (br, OH, NH), 1738, 1651 (C═O).

¹H-NMR (CDCl₃) δ: 0.628 (3H, s), 1.032 (3H, s), 1.96-2.05 (2H, m), 2.633 (1H, dd, J=5.8, 14.6 Hz), 2.852 (1H, dd, J=8.0, 14.6 Hz), 3.135 (1H, d, J=11.6 Hz), 3.338 (1H, d, J=14.2 Hz), 3.436 (2H, q, J=6.6 Hz), 3.567 (2H, s), 3.604 (3H, s), 3.648 (1H, d, J=12.2 Hz), 3.56-3.68 (1H, m), 3.890 (3H, s), 3.988 (2H, t, J=6.6 Hz), 4.156 (1H, dd, J=4.2, 11.6 Hz), 4.38-4.47 (2H, m), 6.05-6.12 (1H, br), 6.150 (1H, s), 6.603 (1H, s), 6.82-7.38 (9H, m).

Elemental analysis (C₃₆H₄₃N₂O₉Cl) Cal'd: C, 63.29; H, 6.34; N, 4.10. Found: C, 63.26; H, 6.35; N, 3.92.

(2) A mixture of methyl 4-[3-[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]aminopropyloxy]phenylacetate obtained in Example 96-(1) (1.18 g, 1.73 mmol), 1 N aqueous sodium hydroxide solution (4 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue purified by recrystallization from ethyl acetate-hexane (1:1) to obtain 4-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]phenylacetic acid (0.95 g, 1.42 mmol, 82%) as colorless prisms.

Melting point 125-128° C.

[α]_{D}²²−147.3° (c=0.20, MeOH).

IR νₘₐₓ (KBr) cm⁻¹: 3600-2400 (br, COOH, OH, NH), 1716, 1651 (C═O).

¹H-NMR (CDCl₃) δ: 0.521 (3H, s), 0.920 (3H, s), 1.82-1.95 (2H, m), 2.529 (1H, dd, J=5.8, 14.2 Hz), 2.749 (1H, dd, =7.6, 14.2 Hz), 3.041 (1H, d, J=11.4 Hz), 3.221 (1H, d, J=14.6 Hz), 3.328 (2H, q, J=6.0 Hz), 3.484 (2H, s) 3.491 (1H, d, J=11.4 Hz), 3.499 (3H, s), 3.786 (3H, s), 3.887 (2H, t, J=6.0 Hz), 4.25-4.33 (2H, m), 6.036 (1H, s), 6.04-6.14 (1H, br), 6.507 (1H, d, J=1.8 Hz), 6.72-7.27 (9H, m).

Elemental analysis (C₃₅H₄₁N₂O₉Cl.0.3H₂O) Cal'd: C, 62.32; H, 6.22; N, 4.15. Found: C, 62.28; H, 6.32; N, 4.01.

### EXAMPLE 97

4-[3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxy]phenylacetic acid

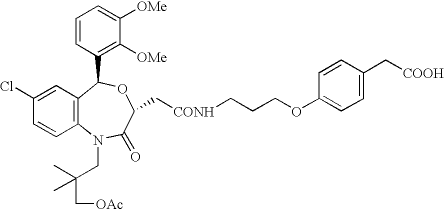

To a mixture of 4-[[3-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]phenylacetic acid obtained in Example 96-(2) (0.5 g, 0.747 mmol), pyridine (0.27 g, 3.36 mmol) and ethyl acetate (5 ml) was added acetyl chloride (0.21 g, 2.62 mmol). After stirring at room temperature for 1 hour, water (4 ml) was added to this mixture, followed by further stirring at room temperature for 3 hours. The organic layer was separated and washed with 1 N hydrochloric acid and saturated saline. This was dried with sodium sulfate and concentrated under reduced pressure. The residue was recrystallized from ethyl acetate-hexane (1:1) to obtain 4-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxy]phenylacetic acid (0.48 g, 0.675 mmol, 90%) as a colorless powder.

Melting point 163-164° C.

[α]_{D}²²−144.8° (c=0.19, MeOH).

IR νₘₐₓ (KBr) cm⁻¹: 3400-2400 (br, COOH, NH), 1732, 1674 (C═O).

¹H-NMR (CDCl₃) δ: 0.923 (3H, s), 0.993 (3H, s), 1.92-2.05 (2H, m), 2.013 (3H, s), 2.626 (1H, dd, J=5.8, 14.4 Hz), 2.833 (1H, dd, J=7.8, 14.4 Hz), 3.415 (2H, q, J=6.2 Hz), 3.477 (1H, d, J=14.4 Hz), 3.577 (2H, s), 3.599 (3H, s), 3.706 (1H, d, J=11.0 Hz), 3.833 (1H, d, J=11.0 Hz), 3.883 (3H, s), 3.960 (2H, t, J=6.0 Hz), 4.388 (1H, dd, J=5.8, 7.8 Hz), 4.499 (1H, d, J=14.4 Hz), 6.16-6.26 (1H, br), 6.244 (1H, s), 6.623 (1H, d, J=2.0 Hz), 6.81-7.36 (9H, m).

Elemental analysis (C₃₇H₄₃N₂O₁₀Cl) Cal'd: C, 62.49; H, 6.09; N, 3.94. Found: C, 62.55; H, 6.17; N, 3.81.

### EXAMPLE 98

4-[3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxybenzoic acid

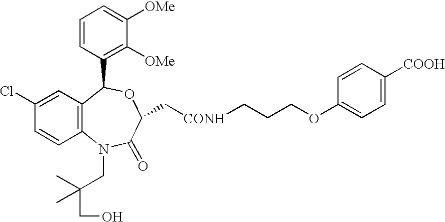

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (1 g, 2.09 mmol) and ethyl 4-(3-aminopropyloxy)benzoate hydrochloride (0.57 g, 2.20 mmol) in N,N-dimethylformamide (10 ml) were added diethyl cyanophosphate (0.38 g, 2.30 mmol) and then triethylamine (0.53 g, 5.23 mmol). The mixture was stirred at room temperature for 30 minutes. The mixture was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was recrystallized from hexane-ethyl acetate (1:1) to obtain ethyl 4-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxybenzoate (1.38 g, 2.02 mmol, 97%) as a colorless powder.

Melting point 172-173° C.

[α]_{D}²²−153.5° (c=0.28, MeOH).

IR νₘₐₓ (KBr) cm⁻¹: 3600-3200 (br, OH, NH), 1709, 1651 (C═O).

¹H-NMR (CDCl₃) δ: 0.630 (3H, s), 1.033 (3H, s), 1.381 (3H, t, J=7.4 Hz), 1.96-2.10 (2H, m), 2.648 (1H, dd, J=5.8, 14.2 Hz), 2.843 (1H, dd, J=7.4, 14.2 Hz), 3.139 (1H, t, J=11.5 Hz), 3.344 (1H, d, J=14.2 Hz), 3.351 (2H, q, J=6.2 Hz), 3.600 (1H, dd, J=3.8, 11.5 Hz), 3.603 (3H, s), 3.886 (3H, s), 4.053 (2H, t, J=5.8 Hz), 4.143 (1H, dd, J=3.8, 11.5 Hz), 4.349 (2H, q, J=7.4 Hz), 4.39-4.46 (2H, m), 6.04-6.10 (1H, br), 6.154 (1H, s), 6.603 (1H, d, J=2.2 Hz), 6.887 (2H, d, J=8.8 Hz), 6.95-7.39 (5H, m), 7.987 (2H, d, J=8.8 Hz).

Elemental analysis (C₃₆H₄₃N₂O₉Cl) Cal'd: C, 63.29; H, 6.34; N, 4.10. Found: C, 62.89; H, 6.45; N, 4.14.

(2) A mixture of ethyl 4-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxybenzoate obtained in Example 98-(1) (1.2 g, 1.76 mmol), 1 N aqueous sodium hydroxide solution (4 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue purified by recrystallization from ethyl acetate-hexane (1:1) to obtain 4-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxybenzoic acid (0.74 g, 1.13 mmol, 64%) as colorless prisms.

Melting point 138-139° C.

[α]_{D}²²−157.6° (c=0.18, MeOH).

IR νₘₐₓ (KBr) cm⁻¹: 3600-2400 (br, COOH, OH, NH), 1651 (C═O).

¹H-NMR (CDCl₃) δ: 0.634 (3H, s), 1.035 (3H, s), 2.00-2.10 (2H, m), 2.665 (1H, dd, J=5.8, 14.2 Hz), 2.856 (1H, dd, =7.4, 14.2 Hz), 3.157 (1H, t, J=12.2 Hz), 3.349 (1H, d, J=14.4 Hz), 3.459 (2H, q, J=5.6 Hz), 3.603 (3H, s) 3.605 (1H, dd, J=12.2 Hz), 3.885 (3H, s), 4.070 (2H, t, J=6.0 Hz), 4.39-4.47 (2H, m), 6.154 (1H, s), 6.12-6.22 (1H, br), 6.602 (1H, d, J=1.8 Hz), 6.88-7.34 (7H, m), 8.015 (2H, d, J=8.8 Hz).

Elemental analysis (C₃₄H₃₉N₂O₉Cl.0.5H₂O) Cal'd: C, 61.49; H, 6.07; N, 4.22. Found: C, 61.53; H, 6.11; N, 3.88.

### EXAMPLE 99

4-[3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxybenzoic acid

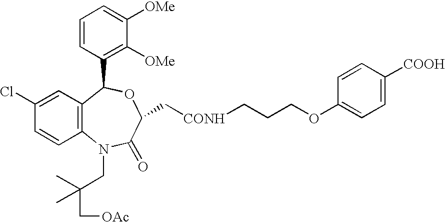

To a mixture of 4-[[3-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxybenzoic acid obtained in Example 98-(2) (0.4 g, 0.611 mmol), pyridine (0.21 g, 2.75 mmol) and ethyl acetate (5 ml) was added acetyl chloride (0.17 g, 2.14 mmol). After stirring at room temperature for 1 hour, water (4 ml) was added to this mixture, followed by further stirring at room temperature for 3 hours. The organic layer was separated and washed with 1 N hydrochloric acid and saturated saline. This was dried with sodium sulfate and concentrated under reduced pressure to obtain 4-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxybenzoic acid (0.33 g, 0.473 mmol, 77%) as a colorless amorphous powder.

[α]_{D}²²−140.7° (c=0.12, MeOH).

IR νₘₐₓ (KBr) cm⁻¹: 3500-2400 (br, COOH, NH), 1732, 1714, 1682 (C═O).

¹H-NMR (CDCl₃) δ: 0.938 (3H, s), 1.000 (3H, s), 1.96-2.10 (2H, m), 2.017 (3H, s), 2.660 (1H, dd, J=5.8, 14.0 Hz), 2.860 (1H, dd, J=7.6, 14.0 Hz), 3.456 (2H, q, J=6.3 Hz), 3.506 (1H, t, J=13.8 Hz), 3.603 (2H, s), 3.709 (1H, d, J=11.0 Hz), 3.852 (1H, dd, J=11.0 Hz), 3.881 (3H, s), 4.061 (2H, t, J=6.0 Hz), 4.407 (1H, dd, J=5.8, 7.6 Hz), 4.517 (1H, d, J=13.8 Hz), 6.253 (1H, s), 6.28-6.38 (1H, br), 6.627 (1H, d, J=2.2 Hz), 6.890 (2H, d, J=8.8 Hz), 6.93-7.36 (5H, m), 7.984 (2H, d, J=8.8 Hz).

Elemental analysis (C₃₆H₄₁N₂O₁₀Cl.0.5H₂O) Cal'd: C, 61.23; H, 5.99; N, 3.97. Found: C, 61.19; H, 5.81; N, 3.81.

### EXAMPLE 100

3-[3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoic acid

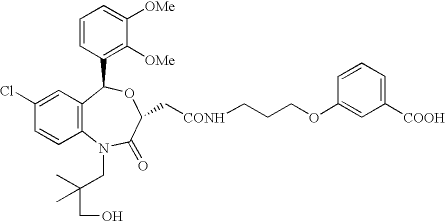

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (1 g, 2.09 mmol) and methyl-3-(3-aminopropyloxy)benzoate hydrochloride (0.54 g, 2.20 mmol) in N,N-dimethylformamide (10 ml) were added diethyl cyanophosphate (0.38 g, 2.30 mmol) and then triethylamine (0.53 g, 5.23 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was recrystallized from ethyl acetate-hexane (1:1) to obtain methyl 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoate (1.28 g, 1.87 mmol, 90%) as colorless prisms.

Melting point 99-100° C.

[α]_{D}²²−154.7° (c=0.19, MeOH).

IR νₘₐₓ (KBr) cm⁻¹: 3500-3200 (br, H, NH), 1720, 1653 (C═O).

¹H-NMR (CDCl₃) δ: 0.628 (3H, s), 1.027 (3H, s), 1.99-2.08 (2H, m), 2.645 (1H, dd, J=6.0, 14.4 Hz), 2.859 (1H, dd, J=7.8, 14.4 Hz), 3.134 (1H, t, J=11.4 Hz), 3.344 (1H, d, J=15.0 Hz), 3.456 (2H, q, J=6.6 Hz), 3.601 (3H, s), 3.603 (1H, dd, J=3.6, 113 Hz), 3.887 (3H, s), 3.916 (3H, s), 4.055 (2H, t, J=5.8 Hz), 4.137 (1H, dd, J=3.6, 11.4 Hz), 4.38-4.47 (2H, m). 6.04-6.12 (1H, br), 6.152 (1H, s), 6.597 (1H, d, J=2.0 Hz), 6.95-7.66 (9H, m).

Elemental analysis (C₃₅H₄₁N₂O₉Cl.H₂O) Cal'd: C, 61.49; H, 6.07; N, 4.22. Found: C, 61.38; H, 6.35; N, 3.81.

(2) A mixture of methyl 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoate obtained in Example 100-(1) (1.3 g, 1.94 mmol), 1 N aqueous sodium hydroxide solution (4 ml) and ethanol (10 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:2) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoic acid (1.09 g, 1.66 mmol, 86%) as colorless prisms.

Melting point 132-134° C.

[α]_{D}²²−161.8° (c=0.24, MeOH).

IR νₘₐₓ (KBr) cm⁻¹: 3600-2400 (br, COOH, OH), 1712, 1651 (C═O).

¹H-NMR (CDCl₃) δ: 0.632 (3H, s), 1.024 (3H, s), 1.96-2.08 (2H, m), 2.665 (1H, dd, J=5.8, 14.6 Hz), 2.867 (1H, dd, J=7.4, 14.6 Hz), 3.160 (1H, d, J=11.8 Hz), 3.351 (1H, d, J=14.4 Hz), 3.469 (2H, q, J=6.0 Hz), 3.597 (3H, s), 3.608 (1H, dd, J=11.8 Hz), 3.879 (3H, s), 4,068 (2H, t, J=6.2 Hz), 4.39-4.46 (2H, m). 6.149 (1H, s), 6.12-6.24 (1H, br), 6.599 (1H, d, J=1.6 Hz), 6.94-7.71 (9H, m).

Elemental analysis (C₃₄H₃₉N₂O₉Cl.0.5H₂O) Cal'd: C, 61.49; H, 6.07; N, 4.22. Found: C, 61.35; H, 6.08; N, 4.13.

### EXAMPLE 101

3-[3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxy]benzoic acid

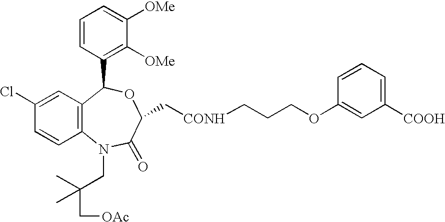

To a mixture of 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminopropyloxy]benzoic acid obtained in Example 100-(2) (0.4 g, 0.611 mmol), pyridine (0.2 g, 2.75 mmol) and ethyl acetate (5 ml) was added acetyl chloride (0.17 g, 2.14 mmol). The mixture was stirred at room temperature for 1 hour and, after addition of water (4 ml), it was further stirred at room temperature for 2 hours. The organic layer was separated, washed with 1 N hydrochloric acid and saturated saline, dried by sodium sulfate and concentrated under reduced pressure to obtain 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminopropyloxy]benzoic acid (0.29 g, 0.416 mmol, 68%) as a colorless amorphous powder.

[α]_{D}²²−150.1° (c=0.19, MeOH).

IR νₘₐₓ (KBr) cm⁻¹: 3600-2400 (br, COOH, OH), 1722, 1676 (C═O).

¹H-NMR (CDCl₃) δ: 0.931 (3H, s), 0.989 (3H, s), 1.96-2.10 (2H, m), 2.015 (3H, s), 2.659 (1H, dd, J=5.6, 13.6 Hz), 2.861 (1H, dd, J=7.4, 13.6 Hz), 3.463 (2H, q, J=6.4 Hz), 3.502 (1H, d, J=14.2 Hz), 3.599 (3H, s), 3.711 (1H, d, J=11.0 Hz), 3.854 (1H, dd, J=11.0 Hz), 3.878 (3H, s), 4.055 (2H, t, J=5.8 Hz), 4.403 (1H, dd, J=5.6, 7.4 Hz), 4.511 (1H, d, J=14.2 Hz), 6.249 (1H, s), 6.22-6.34 (1H, br), 6.623 (1H, d, J=1.8 Hz), 6.93-7.70 (9H, m).

Elemental analysis (C₃₆H₄₁N₂O₁₀Cl) Cal'd: C, 62.02; H, 5.93; N, 4.02. Found: C, 61.72; H, 5.96; N, 3.95.

### EXAMPLE 102

3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxybenzoic acid

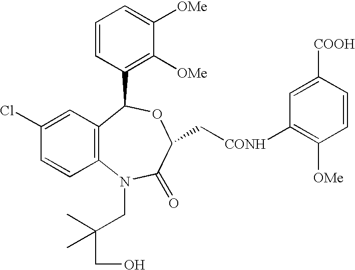

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 3-amino-4-methoxybenzoate hydrochloride (0.46 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel chromatography [eluent:ethyl acetate-hexane (1:1)] to obtain methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxybenzoate (0.68 g, 0.995 mmol, 52%) as colorless needles.

Melting point 138-140° C.

[α]_{D}²²−176.0° (c=0.14, MeOH).

IR νₘₐₓ (KBr) cm⁻¹: 3335 (NH), 1716, 1678 (C═O).

¹H-NMR (CDCl₃) δ: 0.954 (3H, s), 1.015 (3H, s), 2.020 (3H, s), 2.870 (1H, dd, J=6.2, 14.8 Hz), 3.037 (1H, dd, J=6.2, 14.8 Hz), 3.543 (1H, d, J=13.8 Hz), 3.609 (3H, s), 3.717 (1H, d, J=11.0 Hz), 3.850 (3H, s), 3.889 (3H, s), 3.85-3.89 (1H, m), 4.464 (1H, t, J=6.2 Hz), 4,573 (1H, d, J=13.8 Hz), 6.299 (1H, s), 6.636 (1H, s), 6.87-7.34 (6H, m), 7.799 (1H, dd, J=2.2, 8.4 Hz), 8.186 (1H, sr), 8.964 (1H, d, J=2.2 Hz).

Elemental analysis (C₃₅H₃₉N₂O₁₀Cl) Cal'd: C, 59.91; H, 5.75; N, 4.37. Found: C, 61.76; H, 5.81; N, 3.97.

(2) A mixture of methyl 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxybenzoate obtained in Example 102-(1) (0.58 g, 0.849 mmol), 1 N aqueous sodium hydroxide solution (2 ml) and ethanol (5 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by column chromatography [eluent:ethyl acetate-methanol (10:1)] and recrystallization from ethanol-hexane (1:10) to obtain 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxybenzoic acid (0.2 g, 0.319 mmol, 38%) as colorless needles.

Melting point 171-173° C.

[α]_{D}²²−171.7° (c=0.14, MeOH).

IR νₘₐₓ (KBr) cm⁻¹: 3600-2400 (br, COOH, NH, OH), 1684, 1660, 1651 (C═O).

¹H-NMR (CDCl₃) δ: 0.654 (3H, s), 1.053 (3H, s), 2.884 (1H, dd, J=5.8, 14.2 Hz), 3.086 (1H, dd, J=7.0, 14.2 Hz), 3.166 (1H, d, J=11.8 Hz), 3.396 (1H, d, J=14.0 Hz), 3.610 (3H, s), 3.638 (1H, d, J=11.8 Hz), 3.892 (3H, s), 4.45-4.52 (2H, m), 6.195 (1H, s), 6.618 (1H, s), 6.90-7.37 (6H, m), 7.849 (1H, dd, J=2.2, 8.8 Hz), 8.160 (1H, s), 8.999 (1H, d, J=2.2 Hz).

Elemental analysis (C₃₂H₃₅N₂O₉Cl.0.8H₂O) Cal'd: C, 59.91; H, 5.75; N, 4.37. Found: C, 59.92; H, 5.65; N, 4.27.

### EXAMPLE 103

3-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxybenzoic acid

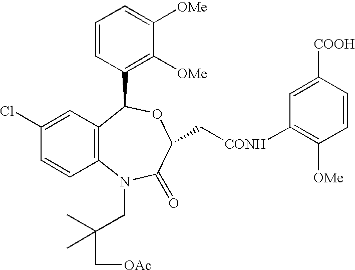

To a mixture of 3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-4-methoxybenzoic acid obtained in Example 102-(2) (0.1 g, 0.159 mmol), pyridine (57 mg, 0.718 mmol) and ethyl acetate (2 ml) was added acetyl chloride (44 mg, 0.558 mmol). After stirring at room temperature for 1 hour, water (2 ml) was added to this mixture, followed by stirring at room temperature for additional 2 hours. The organic layer was separated and washed with 1 N hydrochloric acid and saturated saline. This was dried with sodium sulfate and concentrated under reduced pressure to obtain 3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-4-methoxybenzoic acid (92 mg, 0.137 mmol, 86%) as a colorless amorphous powder.

[α]_{D}²²−176.2° (c=0.16, MeOH).

IR νₘₐₓ (KBr) cm⁻¹: 3600-2400 (br, COOH, NH), 1682 (C═O).

¹H-NMR (CDCl₃) δ: 0.954 (3H, s), 1.016 (3H, s), 2.022 (3H, s), 2.875 (1H, dd, J=6.2, 15.0 Hz), 3.049 (1H, dd, J=7.0, 15.0 Hz), 3.553 (1H, d, J=14.4 Hz), 3.610 (3H, s), 3.719 (1H, d, J=11.0 Hz), 3.874 (1H, d, J=11.0 Hz), 3.868 (3H, s), 3.892 (3H, s), 4.478 (1H, t, J=6.2, 7.0 Hz), 4.578 (1H, d, J=14.4 Hz), 6.305 (1H, s), 6.643 (1H, s), 6.89-7.34 (6H, m), 7.846 (1H, dd, J=2.0, 8.6 Hz), 8.189 (1H, s), 9.025 (1H, s, J=2.0 Hz).

Elemental analysis (C₃₄H₃₇N₂O₁₀Cl.0.5H₂O) Cal'd: C, 60.22; H, 5.65; N, 4.13. Found: C, 60.28; H, 5.71; N, 4.16.

### EXAMPLE 104

4-[2-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetylamino]ethyl]benzoic acid

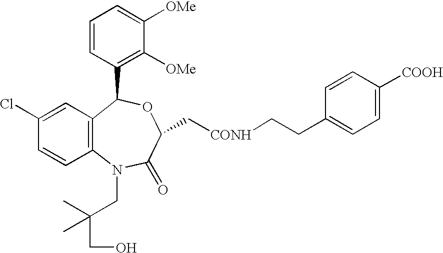

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (0.7 g, 1.46 mmol) and methyl 4-(2-aminoethyl)benzoate hydrochloride (0.33 g, 1.54 mmol) in N,N-dimethylformamide (7 ml) were added diethyl cyanophosphate (0.26 g, 1.61 mmol) and then triethylamine (0.37 g, 3.65 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by column chromatography [eluent:ethyl acetate-hexane (2:1)] and then recrystallization from ether-hexane (1:1) to obtain methyl 4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetylamino]ethyl]benzoate (0.62 g, 0.970 mmol, 66%) as a colorless powder.

Melting point 167-169° C.

[α]_{D}²²−161.3° (c=0.20, MeOH).

IR νₘₐₓ (KBr) cm⁻¹: 3600-3200 (br, NH, OH), 1720, 1653 (C═O).

¹H-NMR (CDCl₃) δ: 0.626 (3H, s), 1.034 (3H, s), 2.591 (1H, dd, J=5.6, 14.4 Hz), 2.812 (1H, dd, J=7.8, 14.4 Hz), 2.864 (2H t, J=7.0 Hz), 3.126 (1H, t, J=11.8 Hz), 3.345 (1H, d, J=14.4 Hz), 3.46-3.57 (3H, m), 3.597 (3H, s), 3.886 (3H, s), 4.142 (1H, dd, J=4.4, 11.8 Hz), 4.34-4.43 (2H, m), 5.82-5.92 (1H, br), 6.128 (1H, s), 6.602 (1H, d, J=1.8 Hz), 7.15-7.36 (7H, m), 7.965 (2H, d, J=8.4 Hz).

Elemental analysis (C₃₄H₃₉N₂O₈Cl) Cal'd: C, 63.89; H, 6.15; N, 4.38. Found: C, 63.67; H, 6.10; N, 4.21.

(2) A mixture of methyl [4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetylamino]ethyl]benzoate obtained in Example 104-(1) (0.52 g, 0.814 mmol), 1 N aqueous sodium hydroxide solution (2 ml) and ethanol (6 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by column chromatography [eluent: ethyl acetate-methanol (5:1)] to obtain 4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetylamino]ethyl]benzoic acid (0.25 g, 0.400 mmol, 49%) as a colorless amorphous powder.

[α]_{D}²²−167.2° (c=0.17, MeOH).

IR νₘₐₓ (KBr) cm⁻¹: 3600-2400 (br, COOH, OH, NH), 1711, 1651 (C═O).

¹H-NMR (CDCl₃) δ: 0.638 (3H, s), 1.042 (3H, s), 2.615 (1H, dd, J=5.6, 14.0 Hz), 2.834 (1H, dd, J=7.2, 14.0 Hz), 2.889 (2H, t, J=6.6 Hz), 3.161 (1H, d, J=12.2 Hz), 3.364 (1H, d, J=14.4 Hz), 3.51-3.62 (3H, m), 3.599 (3H, s), 3.885 (3H, s), 4.37-4.45 (2H, m), 5.96-6.06 (1H, br), 6.122 (1H, s), 6.602 (1H, s), 6.96-7.35 (7H, m), 8.007 (2H, d, J=8.4 Hz).

Elemental analysis (C₃₃H₃₇N₂O₈Cl.0.5H₂O) Cal'd: C, 62.51; H, 6.04; N, 4.42. Found: C, 62.67; H, 6.22; N, 4.46.

### EXAMPLE 105

4-[2-[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetylamino]ethyl]benzoic acid

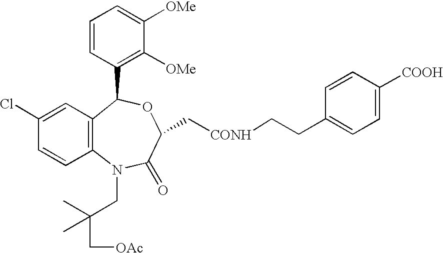

To a mixture of 4-[2-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyamino]ethyl]benzoic acid obtained in Example 104-(2) (0.15 g, 0.240 mmol), pyridine (85 mg, 1.08 mmol) and ethyl acetate (3 ml) was added acetyl chloride (66 mg, 0.840 mmol). After stirring at room temperature for 1 hour, water (3 ml) was added to this mixture, followed by stirring at room temperature for additional 1 hours. The organic layer was separated and washed with 1 N hydrochloric acid and saturated saline with saturated saline. This was dried with sodium sulfate and concentrated under reduced pressure to obtain 4-[2-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetylamino]ethyl]benzoic acid (0.11 g, 0.165 mmol, 69%) as a colorless amorphous powder.

[α]_{D}²²−158.3° (c=0.23, MeOH).

IR νₘₐₓ (KBr) cm⁻¹: 3400-2400 (br, COOH, NH), 1714, 1682 (C═O).

¹H-NMR (CDCl₃) δ: 0.938 (3H, s), 1.005 (3H, s), 2.027 (3H, s), 2.613 (1H, dd, J=5.6, 14.4 Hz), 2.79-2.92 (3H, m), 3.48-3.55 (3H, m), 3.603 (3H, s), 3,715 (1H, d, J=11.0 Hz), 3.885 (3H, s), 4.380 (1H, dd, J=5.6, 8.2 Hz), 4.500 (1H, d, J=14.0 Hz), 6.12-6.20 (1H, br), 6.238 (1H, s), 6.627 (1H, s), 6.95-7.32 (7H, m), 7.982 (2H, d, J=8.0 Hz).

Elemental analysis (C₃₅H₃₉N₂O₉Cl) Cal'd: C, 63.01; H, 5.89; N, 4.20. Found: C, 62.73; H, 6.11; N, 3.95.

### EXAMPLE 106

3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorobenzoic acid

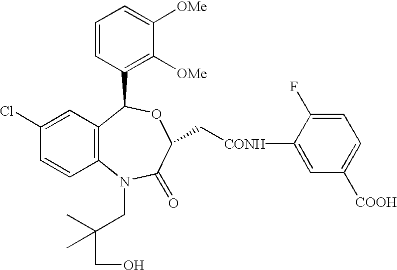

(1) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.03 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of methyl 3-amino-4-fluorobenzoate hydrochloride (0.43 g, 2.11 mmol), triethylamine (0.48 g, 4.80 mmol) and tetrahydrofuran (10 ml). After stirring at room temperature for 30 minutes, water was added thereto and tetrahydrofuran was distilled off. The residue was diluted with ethyl acetate (50 ml), washed with 1 N hydrochloric acid and saturated saline, dried with sodium sulfate, and then concentrated under reduced pressure. The residue was purified by silica gel chromatography [eluent:ethyl acetate-hexane (1:1)] to obtain methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorobenzoate (0.88 g, 1.31, 68%) as a colorless amorphous powder.

[α]_{D}²²−108.3° (c=0.21, MeOH).

IR νₘₐₓ (KBr) cm⁻¹: 3321 (NH), 1728, 1682 (C═O).

¹H-NMR (CDCl₃) δ: 0.958 (3H, s), 1.020 (3H, s), 2.022 (3H, s), 2.881 (1H, dd, J=5.8, 14.6 Hz), 3.077 (1H, dd, J=7.0, 14.6 Hz), 3.549 (1H, d, J=14.2 Hz), 3.621 (3H, s), 3.722 (1H, d, J=11.0 Hz), 3.874 (1H, d, J=11.0 Hz), 3.888 (3H, s), 3.892 (3H, s), 4.423 (1H, dd, J=5.8, 7.0 Hz), 4,582 (1H, d, J=14.2 Hz), 6.304 (1H, s), 6.659 (1H, d, J=2.0 Hz), 6.96-7.39 (6H, m), 7.74-7.86 (1H, m), 8.134 (1H, br), 8.90-8.94 (1H, m).

Elemental analysis (C₃₄H₃₆N₂O₉ClF) Cal'd: C, 60.85; H, 5.41; N, 4.17. Found: C, 60.73; H, 5.72; N, 4.39.

(2) A mixture of methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorobenzoate obtained in Example 106-(1) (0.78 g, 1.16 mmol), 1 N aqueous sodium hydroxide solution (2.5 ml) and ethanol (10 ml) was stirred at 60° C. for 1 hour. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorobenzoic acid (0.43 g, 0.699 mmol, 60%) as colorless needles.

Melting point 163-166° C.

[α]_{D}²²−108.6° (c=0.15, MeOH). IR νₘₐₓ (KBr) cm⁻¹: 3600-2400 (br, COOH, NH, OH), 1711, 1676, 1655 (C═O).

¹H-NMR (CDCl₃) δ: 0.667 (3H, s), 1.043 (3H, s), 2.905 (1H, dd, J=5.4, 15.0 Hz), 3.110 (1H, dd, J=7.2, 15.0 Hz), 3.155 (1H, d, J=11.8 Hz), 3.415 (1H, d, J=13.8 Hz), 3.609 (1H, d, J=11.8 Hz), 3.610 (3H, s), 3.894 (3H, s), 4.44-4.52 (2H, m), 6.197 (1H, s), 6.623 (1H, s), 6.97-7.37 (6H, m), 7.76-7.84 (1H, m), 8.381 (1H, br), 8.828 (1H, d, J=7.0 Hz).

Elemental analysis (C₃₁H₃₂N₂O₈ClF) Cal'd: C, 60.54; H, 5.24; N, 4.55. Found: C, 60.35; H, 5.56; N, 4.38.

### EXAMPLE 107

3-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorobenzoic acid

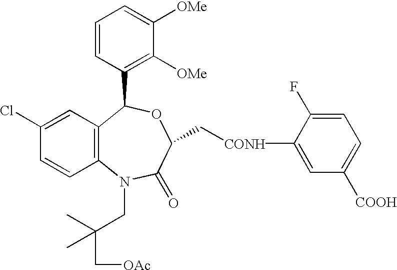

To a mixture of 3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorobenzoic acid obtained in Example 106-(2) (0.2 g, 0.325 mmol), pyridine (0.12 g, 1.46 mmol) and ethyl acetate (3 ml) was added acetyl chloride (89 mg, 1.14 mmol). After stirring at room temperature for 1 hour, water (3 ml) was added to this mixture, followed by stirring at room temperature for additional 2 hours. The organic layer was separated and washed with 1 N hydrochloric acid and saturated saline. This was dried with sodium sulfate and concentrated under reduced pressure. The residue was purified by recrystallization from ethyl acetate-hexane (1:1) to obtain 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorobenzoic acid (0.17 g, 0.259 mmol, 80%) as colorless needles.

Melting point 146-149° C.

[α]_{D}²²−105.0° (c=0.14, MeOH).

IR νₘₐₓ (KBr) cm⁻¹: 3600-2400 (br, COOH, NH), 1743, 1724, 1709, 1684, 1649 (C═O).

¹H-NMR (CDCl₃) δ: 0.963 (3H, s), 1.013 (3H, s), 2.024 (3H, s), 2.901 (1H, dd, J=6.0, 14.2 Hz), 3.073 (1H, dd, J=7.0, 14.2 Hz), 3.554 (1H, d, J=14.0 Hz), 3.610 (3H, s), 3.724 (1H, d, J=11.0 Hz), 3.867 (1H, d, J=11.0 Hz), 3.894 (3H, s), 4.444 (1H, dd, J=6.0, 7.0 Hz), 4.572 (1H, d, J=14.0 Hz), 6.290 (1H, s), 6.640 (1H, s), 6.97-7.35 (6H, m), 7.74-7.83 (1H, m), 8.532 (1H, br), 8.825 (1H, d, J=8.0 Hz).

Elemental analysis (C₃₃H₃₄N₂O₉ClF) Cal'd: C, 60.32; H, 5.22; N, 4.26. Found: C, 60.04; H, 5.32; N, 4.05.

### EXAMPLE 108

5-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid

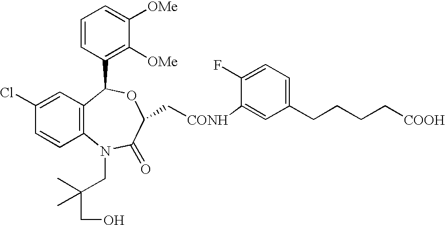

(1) A solution of triethyl 4-phosphonocrotonate (3.8 g, 15 mmol) and 4-fluoro-nitrobenzaldehyde (2.5 g, 15 mmol) in tetrahydrofuran (30 ml) was added to a mixture of sodium hydride (0.40 g, 16.5 mmol) and tetrahydrofuran (30 ml) at 0° C. After stirring at room temperature for 1 hour, the reaction was quenched with water. The reaction mixture was diluted with ethyl acetate (50 ml), washed with 0.5 N hydrochloric acid (35 ml) and saturated saline, dried with anhydrous sodium sulfate, and then concentrated under reduced pressure. The residue was purified by silica gel column chromatography [eluent:hexane-ethyl acetate (3:1)] and recrystallization from ethyl acetate-hexane to obtain ethyl 5-(4-fluoro-3-nitrophenyl)pentane-2,4-dienoate (0.56 g, 2.11 mmol, 14%) as a yellow powder.

Melting point 106-107° C.

IR νₘₐₓ (KBr) cm⁻¹: 1712 (C═O).

¹H-NMR (CDCl₃) δ: 1.328 (3H, t, J=7.0 Hz), 4.248 (2H, q, J=7.0 Hz), 6.077 (1H, d, J=15.0 Hz), 6.80-6.98 (2H, m), 7.300 (1H, dd, J=8.4, 10.2 Hz), 7.36-7.49 (1H, m), 7.710 (1H, ddd, J=2.4, 4.2, 8.4 Hz), 8.146 (1H, dd, J=2.4, 7.2 Hz).

Elemental analysis (C₁₃H₁₂NO₄F) Cal'd: C, 58.87; H, 4.56; N, 5.28. Found: C, 58.91; H, 4.59; N, 5.25.

(2) 10% palladium-carbon (0.1 g) was added to a solution of ethyl 5-(4-fluoro-3-nitrophenyl)pentane-2,4-dienoate obtained in Example 108-(1) (0.46 g, 1.73 mmol) in ethyl acetate (10 ml) and the mixture was subjected to catalytic reduction under normal pressure for 2 hours. The catalyst was removed by filtration and the filtrate was concentrated under reduced pressure. The residue was dissolved in ethyl acetate (50 ml) and to the solution was added 4 N hydrogen chloride solution in ethyl acetate (1 ml), followed by concentration under reduced pressure. The residue was washed with ethyl acetate-hexane (1:1) to obtain ethyl 5-(3-amino-4-fluorophenyl)pentanoate hydrochloride (0.34 g, 1.23 mmol, 71%) as colorless plates.

Melting point 123-124° C.

IR νₘₐₓ (KBr) cm⁻¹: 3200-2400 (br, NH₃⁺), 1712 (C═O).

¹H-NMR (CD₃OD) δ: 1.225 (3H, t, J=7.0 Hz), 1.58-1.68 (4H, m) 2.31-2.38 (2H, m), 4.100 (2H, q, J=7.0 Hz), 7.23-7.32 (3H, m).

Elemental analysis (C₁₃H₁₈NO₂F.HCl) Cal'd: C, 56.62; H, 6.95; N, 5.08. Found: C, 56.63; H, 6.87; N, 5.12.

(3) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (0.41 g, 0.788 mmol) in N,N-dimethylformamide (2 ml) was added triethylamine (0.082 g, 0.812 mmol) at room temperature. To the mixture was added dropwise isobutyl chloroformate (0.13 g, 0.952 mmol) over 10 minutes with ice-cooling in a stream of nitrogen, followed by stirring with ice-cooling as such for 30 minutes. Then, ethyl 5-(3-amino-4-fluorophenyl)pentanoate hydrochloride obtained in Example 108-(2) (0.24 g, 0.870 mmol) was added thereto and pyridine (0.099 g, 1.25 mmol) was added dropwise. After rising to room temperature, the reaction mixture was stirred for 1 hour, followed by addition of water (50 ml) and 1 N hydrochloric acid (2 ml), and extraction twice with ethyl acetate (each 50 ml). The combined organic layer was washed with 5% aqueous potassium hydrogen sulfate solution, saturated aqueous sodium hydrogen carbonate and saturated saline, dried with sodium sulfate, and then concentrated under reduced pressure. The residue was purified by silica gel chromatography [eluent: hexane-ethyl acetate (3:2)] to obtain ethyl 5-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluoropentanoate (0.30 g, 0.405 mmol, 51%) as a colorless amorphous powder.

[α]_{D}²²−130.9° (c=0.15, MeOH).

IR νₘₐₓ (KBr) cm⁻: 3333(NH), 1736, 1680 (C═O).

¹H-NMR (CDCl₃) δ: 0.956 (3H, s), 1.024 (3H, s), 1.240 (3H, t, J=7.4 Hz), 1.55-1.65 (4H, m), 2.030 (3H, s), 2.26-2.34 (2H, m), 2.54-2.61 (2H, m), 2.852 (1H, dd, J=5.8, 14.6 Hz), 3.065 (1H, dd, J=7.4, 14.6 Hz), 3.549 (1H, d, J=14.4 Hz), 3.621 (3H, s), 3.723 (1H, d, J=11.2 Hz), 3.871 (1H, d, J=11.2 Hz), 3.894 (3H, s), 4.112 (2H, q, J=7.4 Hz), 4.411 (1H, dd, J=5.8, 7.4 Hz), 4,584 (1H, d, J=14.4 Hz), 6.296 (1H, s), 6.655 (1H, d, J=2.0 Hz), 6.80-7.41 (7H, m), 8.092 (1H, d, J=7.8 Hz).

(4) A mixture of ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]pentanoate obtained in Example 108-(3) (0.20 g, 0.270 mmol), 1 N aqueous sodium hydroxide solution (0.6 ml) and ethanol (2 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure to obtain 5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid (0.068 g, 0.101 mmol, 38%) as a colorless powder.

Melting point 115-118° C.

[α]_{D}²²−126.6° (c=0.14, MeOH).

IR νₘₐₓ (KBr) cm⁻¹: 3600-2400 (br, COOH, NH, OH), 1657 (C═O).

¹H-NMR (CDCl₃) δ: 0.655 (3H, s), 1.052 (3H, s), 1.63-1.66 (4H, m), 2.33-2.37 (2, m), 2.56-2.60 (2H, m), 2.867 (1H, dd, J=5.7, 14.7 Hz), 3.085 (1H, dd, J=7.2, 14.7 Hz), 3.169 (1H, d, J=12.0 Hz), 3.398 (1H, d, J=13.8 Hz), 3.619 (3H, s), 3.621 (1H, d, J=12.0 Hz), 3.895 (3H, s), 4.435 (1H, dd, J=5.7, 7.2 Hz), 4.495 (1H, d, J=13.8 Hz), 6.193 (1H, s), 6.631 (1H, d, J=2.1 Hz), 6.84-7.40 (7H, m), 7.915 (1H, brs), 8.055 (1H, d. J=6.9 Hz).

Elemental analysis (C₃₅H₄₀N₂O₈ClF.AcOEt) Cal'd: C, 61.70; H, 6.37; N, 3.69. Found: C, 61.42; H, 6.30; N, 3.69.

### EXAMPLE 109

3-[[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenylacetic acid

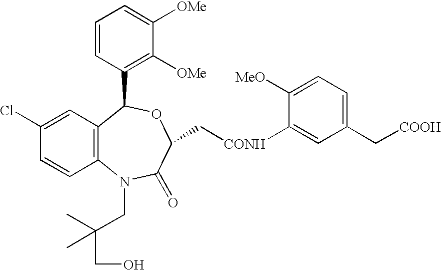

(1) A mixture of 4-hydroxy-3-nitrophenylacetic acid (10 g, 50.7 mmol), sodium hydride (2.6 g, 0.11 mol), iodomethane (15.6 g, 0.11 mol) and N,N-dimethylformamide (170 ml) was stirred at room temperature overnight. The mixture was diluted with water (200 ml) and extracted with ethyl acetate (200 ml). The extract was washed with 1 N aqueous sodium hydroxide solution, 5% potassium hydrogen sulfate, saturated aqueous sodium hydrogen sulfate and saturated saline, dried with anhydrous sodium sulfate, and then concentrated under reduced pressure. The residue was recrystallized from ethyl acetate-hexane (1:1) to obtain methyl 2-(4-methoxy-3-nitrophenyl)acetate (10.4 g, 46.2 mmol, 91%) as colorless needles.

Melting point 101-102° C.

IR νₘₐₓ (KBr) cm⁻¹: 1730 (C═O).

¹H-NMR (CDCl₃) δ: 3.626 (2H, s), 3.718 (3H, s), 3.960 (3H, s), 7.062 (1H, d, J=8.8 Hz), 7.478 (1H, dd, J=2.2, 8.8 Hz), 7.795 (1H, d, J=2.2 Hz).

Elemental analysis (C₁₀H₁₁NO₅) Cal'd: C, 55.58; H, 5.30; N, 4.96. Found: C, 53.44; H, 4.87; N, 5.98.

(2) 10% palladium-carbon (0.3 g) and 4 N hydrogen chloride solution in ethyl acetate (3 ml) were added to a solution of ethyl 2-(4-methoxy-3-nitrophenyl)acetate obtained in Example 109-(1) (2.5 g, 11.1 mmol) in methanol (50 ml) and the mixture was subjected to catalytic reduction under normal pressure for 3 hours. The catalyst was removed by filtration and the filtrate was concentrated under reduced pressure. The residue was dissolved in ethyl acetate (50 ml) and to the solution was added 4 N hydrogen chloride solution in ethyl acetate (3 ml), followed by concentration under reduced pressure. The residue was washed with ethyl acetate-hexane (1:1) to obtain ethyl 2-(3-amino-4-methoxyphenyl)acetate hydrochloride (2.5 g, 10.8 mmol, 97%) as a colorless powder.

Melting point 190-195° C.

IR νₘₐₓ (KBr) cm⁻¹: 3200-2400 (br, NH₃⁺), 1739 (C═O).

¹H-NMR (CD₃OD) δ: 3.661 (2H, s), 3.681 (3H, s), 3.967 (3H, s), 7.169 (1H, d, J=8.4 Hz), 7,30-7.39 (2H, m).

Elemental analysis (C₁₀H₁₃NO₃.HCl.0.1H₂O) Cal'd: C, 55.58; H, 5.30; N, 4.96. Found: C, 51.14; H, 5.98; N, 5.96.

(3) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1 g, 0.577 mmol) in N,N-dimethylformamide (5 ml) was added triethylamine (0.20 g, 2.02 mmol) at room temperature. To the mixture was added dropwise isobutyl chloroformate (0.31 g, 2.30 mmol) over 10 minutes with ice-cooling in a stream of nitrogen, followed by stirring with ice-cooling as such for 30 minutes. Then, ethyl 2-(3-amino-4-methoxyphenyl)acetate hydrochloride obtained in Example 109-(2) (0.49 g, 2.11 mmol) was added thereto and pyridine (0.099 g, 1.25 mmol) was added dropwise. After rising to room temperature, the reaction mixture was stirred for 1 hour, followed by addition of water (50 ml) and 1 N hydrochloric acid (4 ml), and extraction twice with ethyl acetate (each 50 ml). The combined organic layer was washed with 5% aqueous potassium hydrogen sulfate solution, saturated aqueous sodium hydrogen carbonate and saturated saline, dried with sodium sulfate, and then concentrated under reduced pressure. The residue was purified by silica gel chromatography [eluent:hexane-ethyl acetate (1:1)] to obtain ethyl 3-[[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]amino]-4-methoxyphenylacetate (0.79 g, 1.13 mmol, 59%) as a colorless amorphous powder.

[α]_{D}²²−174.2° (c=0.12, MeOH).

IR νₘₐₓ (KBr) cm⁻¹: 3337 (NH), 1736, 1682 (C═O).

¹H-NMR (CDCl₃) δ: 0.952 (3H, s), 1.020 (3H, s), 2.028 (3H, s), 2.849 (1H, dd, J=5.8, 14.6 Hz), 3.036 (1H, dd, J=6.6, 14.6 Hz), 3.541 (1H, d, J=13.8 Hz), 3.562 (2H, s), 3.610 (3H, s), 3.669 (3H, s), 3.720 (1H, d, J=11.4 Hz), 3.788 (3H, s), 3.872 (1H, d, J=11.4 Hz), 3.890 (3H, s), 4.445 (1H, dd, J=5.8, 6.6 Hz), 4.579 (1H, d, J=13.8 Hz), 6.292 (1H, s), 6.645 (1H, s), 6.79-7.34 (7H, m), 8.193 (1H, brs), 8.272 (1H, d, J=2.2 Hz).

Elemental analysis (C₃₆H₄₁N₂O₁₀Cl) Cal'd: C, 55.58; H, 5.30; N, 4.96. Found: C, 61.98; H, 6.05; N, 3.88.

(4) A mixture of ethyl 3-[[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]amino]-4-methoxyphenyl]acetate obtained in Example 109-(3) (0.69 g, 0.990 mmol), 1 N aqueous sodium hydroxide solution (2.5 ml) and ethanol (7 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure to obtain 5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]acetic acid (0.51 g, 0.795 mmol, 80%) as a colorless powder.

Melting point 215-216° C. (AcOEt-hexane).

[α]_{D}²²−186.0° (c=0.16, MeOH).

IR νₘₐₓ (KBr) cm⁻¹: 3600-2400 (br, COOH, OH, NH), 1728, 1658 (C═O).

¹H-NMR (CDCl₃) δ: 0.646 (3H, s), 1.047 (3H, s), 2.854 (1H, dd, J=6.0, 14.7 Hz), 3.071 (1H, dd, J=7.2, 14.7 Hz), 3.160 (1H, d, J=12.3 Hz), 3.384 (1H, d, J=14.7 Hz), 3.578 (2H, s), 3.606 (3H, s), 3.626 (1H, d, J=12.3 Hz), 3.813 (3H, s), 4.42-4.49 (2H, m), 6.180 (1H, s), 6.616 (1H, d, J=1.5 Hz), 6.81-7.36 (7H, m), 8.196 (1H, br), 8.251 (1H, d. J=1.8 Hz).

Elemental analysis (C₃₃H₃₇N₂O₉Cl) Cal'd: C, 61.82; H, 5.82; N, 4.73. Found: C, 61.47; H, 5.81; N, 4.22.

### EXAMPLE 110

4-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]butanoic acid

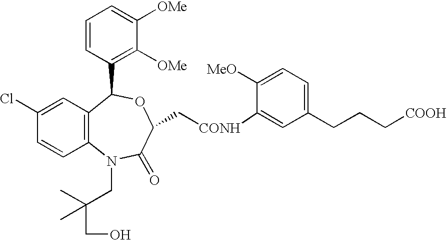

(1) To a solution of (4-methoxy-3-nitrophenyl)acetic acid (8 g, 37.9 mmol) in tetrahydrofuran (80 ml) was added carbonyldiimidazole (6.8 g, 41.7 mmol). After stirring at room temperature for 1.5 hours, magnesium chloride (3.6 g, 37.9 mmol) and potassium salt of monoethyl malonate (6.5 g, 37.9 mmol) were added thereto. The mixture was stirred at 60° C. for 1 hour. Then, the reaction mixture was diluted with ethyl acetate (100 ml) and washed with 1 N hydrochloric acid, saturated aqueous sodium hydrogen carbonate solution and saturated saline. After drying with sodium sulfate, the mixture was concentrated under reduced pressure. The residue was purified by silica gel column chromatography [eluent:hexane-ethyl acetate (1:1)] to obtain ethyl 4-(4-methoxy-3-nitrophenyl)-3-oxobutanoate (7.8 g, 27.7 mmol, 73%) as pale yellow needles.

Melting point 87-88° C. (AcOEt-hexane).

IR νₘₐₓ (KBr) cm⁻¹: 1743, 1720 (C═O).

¹H-NMR (CDCl₃) δ: 1.289 (3H, t, J=7.2 Hz), 3.511 (2H, s), 3.869 (2H, s), 3.962 (3H, s), 4.211 (2H, q, J=7.2 Hz), 7.075 (1H, d, J=8.7 Hz), 7.394 (1H, dd, J=1.8, 8.7 Hz), 7.715 (1H, d, J=1.8 Hz).

Elemental analysis (C₁₃H₁₁NO₆) Cal'd: C, 55.51; H, 5.38; N, 4.98. Found: C, 55.58; H, 5.30; N, 4.96.

(2) To a solution of ethyl 4-(4-methoxy-3-nitrophenyl)-3-oxobutanoate obtained in Example 110-(1) (7.5 g, 26.7 mmol) in methanol (80 ml) was added sodium borohydride (1.1 g, 29.3 mmol) at −20° C. After stirring at 0° C. for 30 minutes, 0.3 N hydrochloric acid (120 ml) was added thereto. The mixture was diluted with ethyl acetate (150 ml) and washed with water, saturated aqueous sodium hydrogen carbonate solution and saturated saline. After drying with sodium sulfate, the residue was purified by silica gel column chromatography [eluent:hexane-ethyl acetate (1:1)] to obtain ethyl 3-hydroxy-4-(4-methoxy-3-nitrophenyl)butanoate (7.4 g, 26.1 mmol, 98%) as a pale yellow oil.

IR νₘₐₓ (KBr) cm⁻¹: 3600-3200 (br, OH), 1728 (C═O).

¹H-NMR (CDCl₃) δ: 1.276 (3H, t, J=7.2 Hz), 2.432 (1H, dd, J=8.4, 16.5 Hz), 2.526 (1H, dd, J=3.6, 16.5 Hz), 2.767 (1H, dd, J=5.7, 14.1 Hz), 2.825 (1H, dd, J=7.2, 14.1 Hz), 3.125 (1H, d, J=3.6 Hz), 3.950 (3H, s), 4.175 (2H, q, J=7.2 Hz), 4.20-4.29 (1H, m), 7.039 (1H, d, J=8.7 Hz), 7.438 (1H, dd, J=2.1, 8.7 Hz), 7.744 (1H, d, J=2.1 Hz).

(3) A mixture of ethyl 3-hydroxy-(4-methoxy-3-nitrophenyl)butanoate obtained in Example 110-(2) (7.0 g, 24.7 mmol), triethylamine (3.0 g, 29.7 mmol), methanesulfonyl chloride (3.1 g, 27.2 mmol) and ethyl acetate (70 ml) was stirred at 0° C. for 30 minutes. 1,8-Diazabicyclo[5.4.0]-7-undecene (4.5 g, 29.7 mmol) was added and the resulting mixture was stirred at 0° C. for 30 minutes. The mixture was diluted with ethyl acetate (100 ml) and washed with 1 N hydrochloric acid (66 ml), saturated aqueous sodium hydrogen carbonate solution, and saturated saline. After drying with sodium sulfate, the mixture was concentrated under reduced pressure. The residue was purified by silica gel column chromatography [eluent: hexane-ethyl acetate (3:1)] to obtain ethyl 4-(4-methoxy-3-nitrophenyl)-2-butanoate (4.7 g, 17.7 mmol, 72%) as a pale yellow oil.

IR νₘₐₓ (KBr) cm⁻¹: 1730 (C═O), 1620 (C═C).

¹H-NMR (CDCl₃) δ: 1.286 (⅖×3H, t, J=7.2 Hz), 1.292 (⅗×3H, t, J=7.2 Hz), 3.249 (⅗×2H, dd, J=1.2, 6.9 Hz), 3.518 (⅗×2H, dd, J=1.2, 6.9 Hz), 3.956 (⅖×3H, s), 3.965 (⅗×3H, s), 4.186 (⅗×2H, q, J=7.2 Hz), 4.193 (⅖×2H, q, J=7.2 Hz), 5.806 (⅖×1H, dt, J=15.6, 1.2 Hz), 6.271 (⅗×1H, dt, J=15.9, 1.2 Hz), 6.434 (⅗×1H, d, J=15.9 Hz), 6.99-7.09 (1H+⅖×1H, m), 7.356 (⅖×1H, dd, J=2.4, 8.7 Hz), 7.540 (⅗×1H, dd, J=2.4, 8.7 Hz), 7.676 (⅖×1H, d, J=2.4 Hz), 7.858 (⅗×1H, d, J=2.4 Hz).

(4) 10% palladium-carbon (0.4 g) and 4 N hydrogen chloride solution in ethyl acetate (5 ml) were added to a solution of ethyl 4-(4-methoxy-3-nitrophenyl)-2-butanoate obtained in Example 110-(3) (4.5 g, 17.0 mmol) in ethanol (100 ml) and the resultant suspension was subjected to catalytic reduction under normal pressure for 5 hours. The catalyst was removed by filtration and the filtrate was concentrated under reduced pressure. The residue was dissolved in ethyl acetate (50 ml) and to the solution was added 4 N hydrogen chloride solution in ethyl acetate (6 ml). The solvent was distilled off and the residue was washed with diethyl ether to obtain ethyl 4-(3-amino-4-methoxyphenyl)butanoate hydrochloride (4.2 g, 15.3 mmol, 90%) as a colorless powder.

Melting point 115-121° C.

IR νₘₐₓ (KBr) cm⁻¹: 3200-2400 (br, NH₃⁺), 1722 (C═O).

¹H-NMR (CD₃OD) δ: 1.236 (3H, t, J=7.2 Hz), 1.892 (2H, quintet, J=7.5 Hz), 2.321 (2H, t, J=7.5 Hz), 2.633 (2H, t, J=7.5 Hz), 3.948 (3H, s), 4.104 (2H, q, J=7.2 Hz), 7.12-7.30 (3H, m).

Elemental analysis (C₁₃H₁₉O₃.HCl.0.2H₂O) Cal'd: C, 56.30; H, 7.41; N, 5.05. Found: C, 56.46; H, 7.23; N, 5.04.

(5) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1 g, 1.92 mmol) in N,N-dimethylformamide (5 ml) was added triethylamine (0.20 g, 2.02 mmol) at room temperature. To the mixture was added dropwise isobutyl chloroformate (0.31 g, 2.30 mmol) over 10 minutes with ice-cooling in a stream of nitrogen, followed by stirring with ice-cooling as such for 30 minutes. Then, methyl 2-(3-amino-4-methoxyphenyl)acetate hydrochloride obtained in Example 109-(2) (0.49 g, 2.11 mmol) was added thereto and pyridine (0.24 g, 3.07 mmol) was added dropwise. After rising to room temperature, the reaction mixture was stirred for 1 hour, followed by addition of water (50 ml) and 1 N hydrochloric acid (4 ml), and extraction twice with ethyl acetate (each 50 ml). The combined organic layer was washed with 5% aqueous potassium hydrogen sulfate solution, saturated aqueous sodium hydrogen carbonate and saturated saline, dried with sodium sulfate, and then concentrated under reduced pressure. The residue was purified by column chromatography [eluent:hexane-ethyl acetate (3:2)] to obtain methyl 4-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]butanoate (0.89 g, 1.20 mmol, 63%) as a colorless amorphous powder.

[α]_{D}²²−160.9° (c=0.27, MeOH).

IR νₘₐₓ (KBr) cm⁻¹: 3346(NH), 1732, 1682 (C═O).

¹H-NMR (CDCl₃) δ: 0.951 (3H, s), 1.021 (3H, s), 1.240 (3H, t, J=7.2 Hz), 1.907 (2H, quintet, J=7.5 Hz), 2.028 (3H, s), 2.288 (2H, t, J=7.5 Hz), 2.573 (2H, t, J=7.5 Hz), 2.856 (1H, dd, J=6.3, 15.0 Hz), 3.026 (1H, dd, J=6.3, 15.0 Hz), 3.545 (1H, d, J=14.1 Hz), 3.608(3H, s), 3.722 (1H, d, J=11.1 Hz), 3.777 (3H, s), 3.866 (1H, d, J=11.1 Hz), 3.889 (3H, s), 4.109 (2H, q, J=7.2 Hz), 4.453 (1H, t, J=6.3 Hz), 4.578 (1H, d, J=14.1 Hz), 6.291 (1H, s), 6.636 (1H, d, J=1.5 Hz), 6.75-7.37 (7H, m), 8.16-8.19 (2H, m).

Elemental analysis (C₃₉H₄₇N₂O₁₀Cl) Cal'd: C, 63.36; H, 6.41; N, 3.79. Found: C, 63.20; H, 6.53; N, 3.74.

(6) A mixture of methyl 4-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]butanoate obtained in Example 110-(5) (0.76 g, 1.03 mmol), 1 N aqueous sodium hydroxide solution (2.5 ml) and ethanol (8 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]butanoic acid (0.53 g, 0.792 mmol, 77%) as colorless prisms.

Melting point 119-121° C.

[α]_{D}²²−169.7° (c=0.24, MeOH).

IR νₘₐₓ (KBr) cm⁻¹: 3600-2400 (br, COOH, OH, NH), 1707, 1657 (C═O).

¹H-NMR (CDCl₃) δ: 0.648 (3H, s), 1.049 (3H, s), 1.924 (2H, quintet, J=7.5 Hz), 2.333 (2H, t, J=7.5 Hz), 2.601 (2H, t, J=7.5 Hz), 2.859 (1H, dd, J=5.4, 14.7 Hz), 3.067 (1H, dd, J=6.9, 14.7 Hz), 3.156 (1H, d, J=12.3 Hz), 3.388 (1H, d, J=14.4 Hz), 3.606 (3H, s), 3.623 (1H, d, J=12.3 Hz), 3.784 (3H, s), 3.890 (3H, s), 4.456 (1H, dd, J=5.4, 6.9 Hz), 4.479 (1H, d, J=14.4 Hz), 6.187 (1H, s), 6.619 (1H, d, J=1.8 Hz), 6.76-7.36 (7H, m), 8.16-8.19 (2H, m).

Elemental analysis (C₃₅H₄₁N₂O₉Cl.0.5 AcOEt) Cal'd: C, 62.31; H, 6.36; N, 3.93. Found: C, 62.09; H, 6.43; N, 3.92.

### EXAMPLE 111

4-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]butanoic acid

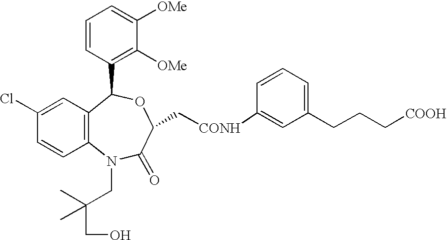

(1) To a solution of 3-nitrophenylacetic acid (10 g, 55.2 mmol) in tetrahydrofuran (100 ml) was added carbonyldiimidazole (10.5 g, 65.0 mmol). After stirring at room temperature for 6 hours, magnesium salt of monoethyl malonate (9.3 g, 32.5 mmol) were added thereto. The mixture was stirred at 60° C. for 3 hour. Then, the reaction mixture was diluted with ethyl acetate (100 ml) and washed with 1 N hydrochloric acid, saturated aqueous sodium hydrogen carbonate solution and saturated saline. After drying with sodium sulfate, the mixture was concentrated under reduced pressure. The residue was purified by silica gel column chromatography [eluent:hexane-ethyl acetate (1:1)] to obtain ethyl 4-(3-nitrophenyl)-3-oxobutanoate (10.0 g, 39.8 mmol, 72%) as a colorless amorphous powder.

IR νₘₐₓ (KBr) cm⁻¹: 1745, 1722 (C═O).

¹H-NMR (CDCl₃) δ: 1.297 (3H, t, J=7.4 Hz), 3.544 (9/10×2H, s), 3.606 (1/10×2H, s), 4.005 (9/10×2H, s), 4.195 (1/10×2H, q, J=7.4 Hz), 4.223 (9/10×2H, q, J=7.4 Hz), 4.982 (1/10×1H, s), 7.52-7.55 (2H, m), 8.08-8.19 (2H, m).

(2) To a solution of ethyl 4-(3-nitrophenyl)-3-oxobutanoate obtained in Example 111-(1) (5.0 g, 19.9 mmol) in methanol (50 ml) was added sodium borohydride (0.95 g, 25.0 mmol) at −78° C. After stirring at −20° C. for 30 minutes, 1 N hydrochloric acid (30 ml) was added thereto. The mixture was diluted with ethyl acetate (300 ml) and washed with water, saturated aqueous sodium hydrogen carbonate solution and saturated saline. After drying with sodium sulfate, the residue was purified by silica gel column chromatography [eluent:hexane-ethyl acetate (2:1)] to obtain ethyl 4-(3-nitrophenyl)-3-hydroxybutanoate (4.5 g, 17.8 mmol, 89%) as a colorless oil.

IR νₘₐₓ (KBr) cm⁻¹: 3600-3200 (br, OH), 1732 (C═O).

¹H-NMR (CDCl₃) δ: 1.278 (3H, t, J=7.4 Hz), 2.452 (1H, dd, J=8.4, 17.0 Hz), 2.562 (1H, dd, J=4.2, 17.0 Hz), 2.90-2.94 82H, m), 3.168 (1H, d, J=4.0 Hz), 4.182 (2H, q, J=7.4 Hz), 4.25-4.36 (1H, m), 7.44-7.62 (2H, m), 8.08-8.13 (2H, m).

(3) A mixture of ethyl 4-(3-nitrophenyl)-3-hydroxybutanoate obtained in Example 111-(2) (4.3 g, 17.0 mmol), triethylamine (2.2 g, 21.4 mmol), methanesulfonyl chloride (2.2 g, 19.6 mmol) and ethyl acetate (40 ml) was stirred at 0° C. for 30 minutes. 1,8-Diazabicyclo[5.4.0]-7-undecene (3.3 g, 21.4 mmol) was added and the resulting mixture was stirred at 0° C. for 30 minutes. The mixture was diluted with ethyl acetate (100 ml) and washed with 6 N hydrochloric acid (12 ml), saturated aqueous sodium hydrogen carbonate solution, and saturated saline. After drying with sodium sulfate, the mixture was concentrated under reduced pressure. The residue was purified by silica gel column chromatography [eluent:hexane-ethyl acetate (5:1)] to obtain ethyl 4-(3-nitrophenyl)-2-butanoate (4.3 g, 18.2 mmol, quant) as a colorless oil.

IR νₘₐₓ (KBr) cm⁻¹: 1732 (C═O).

¹H-NMR (CDCl₃) δ: 1.299 (3H, t, J=7.0 Hz), 3.294 (2H, d, J=5.6 Hz), 4.199 (2H, q, J=7.0 Hz), 6.441 (1H, dd, J=5.6, 16.0 Hz), 6.572 (1H, d, J=16.0 Hz), 7.482 (1H, t, J=8.2 Hz), 7.66-8.23 (3H, m).

(4) 10% palladium-carbon (0.4 g) was added to a solution of ethyl 4-(3-nitrophenyl)-2-butanoate obtained in Example 111-(3) (4.0 g, 17.0 mmol) in ethyl acetate (80 ml) and the resultant suspension was subjected to catalytic reduction at room temperature under normal pressure for 8 hours. The catalyst was removed by filtration and the filtrate was concentrated under reduced pressure. The residue was diluted with ethyl acetate (50 ml) and to the solution was added 4 N hydrogen chloride solution in ethyl acetate (6 ml). The solvent was distilled off and the residue was washed with diethyl ether to obtain ethyl 4-(3-aminophenyl)butanoate hydrochloride (4.0 g, 16.4 mmol, 90%) as a colorless oil.

IR νₘₐₓ (KBr) cm⁻¹: 3200-2400 (br, NH₃⁺), 1730, 1714 (C═O).

¹H-NMR (CD₃OD) δ: 1.245 (3H, t, J=7.0 Hz), 1.930 (2H, quintet, J=7.3 Hz), 2.311 (2H, t, J=7.3 Hz), 2.665 (2H, t, J=7.3 Hz), 4.118 (2H, q, J=7.0 Hz), 7.20-7.37 (3H, m).

Elemental analysis (C₁₂H₁₈NO₂Cl) Cal'd: C, 59.14; H, 7.44; N, 5.75. Found: C, 58.76; H, 7.46; N, 5.71.

(5) To a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.0 g, 1.92 mmol) and N,N-dimethylformamide (0.02 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (5 ml) and added to a mixture of ethyl 4-(3-aminophenyl)butanoate hydrochloride obtained in Example 11-(4) (0.49 g, 2.01 mmol), triethylamine (0.5 g, 5.05 mmol) and tetrahydrofuran (10 ml). After stirring at room temperature for 30 minutes, water was added and tetrahydrofuran was distilled off. The residue was diluted with ethyl acetate (100 ml). This was washed with 1 N hydrochloric acid, saturated aqueous sodium hydrogen carbonate solution and saturated saline, dried with sodium sulfate and concentrated under reduced pressure. The residue was purified by silica gel column chromatography [eluent:hexane-ethyl acetate (3:2)] to obtain ethyl 4-[3-[[[(3R,5S)-7-chloro-5-(2,3-dimethoxyphenyl)-1(3-acetoxy-2,2-dimethylpropyl)-2-oxo-1,2,3,5-tetrahydro-4,1-benzoxazepin-3-yl]acetyl]amino]phenyl]butanoate (0.81 g, 1.14 mmol, 59%) as a colorless amorphous powder.

[α]_{D}²²−133.8° (c=0.45, MeOH).

IR νₘₐₓ (KBr) cm⁻¹: 3327(NH), 1732, 1682 (C═O).

¹H-NMR (CDCl₃) δ: 0.958 (3H, s), 1.026 (3H, s), 1.251 (3H, t, J=7.0 Hz), 1.87-1.97 (2H, m), 2.024 (3H, s), 2.313 (2H, t, J=7.2 Hz), 2.630 (2H, t, J=7.2 Hz), 2.814 (1H, dd, J=5.8, 13.8 Hz), 2.990 (1H, dd, J=7.2, 13.8 Hz), 3.541 (1H, d, J=13.8 Hz), 3.619 (3H, s), 3.731 (1H, d, J=11.0 Hz), 3.872 (1H, d, J=11.0 Hz), 3.894 (3H, s), 4.125 (2H, q, J=7.0 Hz), 4.412 (1H, dd, J=5.8, 7.2 Hz), 4.565 (1H, d, J=13.8 Hz), 6.301 (1H, s), 6.644 (1H, d, J=2.0 Hz), 6.91-7.36 (9H, m), 7.793 (1H, s).

Elemental analysis (C₃₈H₄₅N₂O₉Cl) Cal'd: C, 64.35; H, 6.40; N, 3.95. Found: C, 64.12; H, 6.50; N, 3.90.

(6) A mixture of ethyl 4-[3-[[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]amino]phenyl]butanoate obtained in Example 110-(5) (0.7 g, 0.987 mmol), 1 N aqueous sodium hydroxide solution (2 ml) and ethanol (7 ml) was stirred at 60° C. for 30 minutes. This was diluted with water (50 ml) and, after acidification, extracted twice with ethyl acetate (each 50 ml). The extract was washed with saturated saline, dried with sodium sulfate and concentrated under reduced pressure. The residue was purified by recrystallization from ethanol-hexane (1:1) to obtain 4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]butanoic acid (0.61 g, 0.954 mmol, 97%) as a colorless powder.

Melting point 119-122° C.

[α]_{D}²²−149.7° (c=0.13, MeOH).

IR νₘₐₓ (KBr) cm⁻¹: 3600-2400 (br, COOH), 1712, 1658 (C═O).

¹H-NMR (CDCl₃) δ: 0.652 (3H, s), 1.044 (3H, s), 1.91-2.05 (2H, m), 2.354 (2H, t, J=7.2 Hz), 2.652 (2H, t, J=7.2 Hz), 2.827 (1H, dd, J=5.8, 14.2 Hz), 2.912 (2H, t, J=7.6 Hz), 3.110 (1H, dd, J=5.4, 15.0 Hz), 3.161 (1H, d, J=11.6 Hz), 3.019 (1H, dd, J=7.6, 14.2 Hz), 3.175 (1H, d, J=12.0 Hz), 3.382 (1H, d, J=14.4 Hz), 3.610 (3H, s), 3.580 (1H, d, J=12.0 Hz), 3.889 (3H, s), 4.439 (1H, dd, J=5.8, 7.6 Hz), 4.473 (1H, d, J=14.4 Hz), 6.189 (1H, s), 6.623 (1H, d, J=1.8 Hz), 6.91-7.36 (9H, m), 7.82-7.90 (1H, br).

Elemental analysis (C₃₄H₃₉N₂O₈Cl.0.1H₂O) Cal'd: C, 63.71; H, 6.16; N, 4.37. Found: C, 63.44; H, 6.28; N, 4.36.

### EXAMPLE 112

3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-5,6,7,8-tetrahydro-1-naphthoic acid

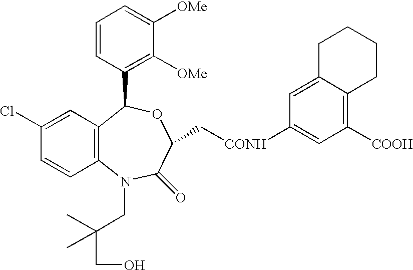

(1) A mixture of 3-nitro-5,6,7,8-tetrahydro-1-naphthoic acid (0.5 g, 2.26 mmol), potassium carbonate (0.40 g, 2.92 mmol), iodomethane (0.35 g, 2.49 mmol) and N,N-dimethylformamide (5 ml) was stirred at room temperature for 1 hour. This mixture was diluted with water and extracted with ethyl acetate (100 ml). The extract was washed with saturated saline, dried with anhydrous sodium sulfate and concentrated under reduced pressure to obtain methyl 3-nitro-5,6,7,8-tetrahydro-1-naphthoate (0.55 g, 2.34 mmol, quant) as a colorless amorphous powder.

IR νₘₐₓ (KBr) cm⁻¹: 1732 (C═O).

¹H-NMR (CDCl₃) δ: 1.80-1.87 (4H, m), 2.88-2.95 (2H, m), 3.12-3.18 (2H,m), 3.931 (3H, s), 8.073 (1H, d, J=2.6 Hz), 8.503 (1H, d, J=2.6 Hz).

(2) 10% palladium-carbon (0.1 g) was added to a solution of methyl 3-nitro-5,6,7,8-tetrahydro-1-naphthoate obtained in Example 112-(1) (0.55 g, 2.34 mmol) in ethyl acetate (20 ml) and the resultant suspension was subjected to catalytic reduction at room temperature under normal pressure for 3 hours. The catalyst was removed by filtration and the filtrate was concentrated under reduced pressure. The residue was diluted with ethyl acetate (50 ml) and to the solution was added 4 N hydrogen chloride solution in ethyl acetate (7 ml), followed by concentration under reduced pressure. The residue was washed with diethyl ether-hexane (1:1) to obtain methyl 3-amino-5,6,7,8-tetrahydro-1-naphthoate (0.48 g, 2.34 mmol, quant) as a colorless oil.

IR νₘₐₓ (KBr) cm⁻¹: 3600-3200 (br, NH₂), 1714 (C═O).

¹H-NMR (CDCl₃) δ: 1.58-1.66 (1H, br), 1.71-1.77 (4H, m), 2.68-2.75 (2H, m), 2.88-2.94 (2H, m), 3.52-3.60 (1H, br), 3.847 (3H, s), 6.577 (1H, d, J=2.6 Hz), 7.026 (1H, d, J=2.6 Hz).

(3) To a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 1-(1) (1.1 g, 2.13 mmol), N,N-dimethylformamide (0.02 ml) and tetrahydrofuran (10 ml) was added thionyl chloride (0.7 g, 5.88 mmol) at room temperature, followed by stirring for 1 hour. The mixture was concentrated under reduced pressure and the residue was dissolved in tetrahydrofuran (10 ml). The solution was added to a mixture of methyl 3-amino-5,6,7,8-tetrahydro-1-naphthoate obtained in Example 112-(2) (0.48 g, 2.34 mmol), triethylamine (0.48 g, 4.80 mmol) and tetrahydrofuran (10 ml). After stirring at room temperature for 30 minutes, the mixture was diluted with ethyl acetate (100 ml). This was washed with 1 N hydrochloric acid, saturated aqueous sodium hydrogen carbonate solution and saturated saline, dried with sodium sulfate and concentrated under reduced pressure. The residue was purified by silica gel column chromatography [eluent:hexane-ethyl acetate (3:2)] to obtain methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-5,6,7,8-tetrahydro-1-naphthoate (1.11 g, 1.57 mmol, 74%) as a colorless amorphous powder.

[α]_{D}²²−118.2° (c=0.27, MeOH).

IR νₘₐₓ (KBr) cm⁻¹: 3323 (NH), 1724, 1682 (C═O).

¹H-NMR (CDCl₃) δ: 0.956 (3H, s), 1.022 (3H, s), 1.72-1.80 (4H, m), 2.78-3.03 (6H, m), 3.533 (1H, d, J=14.0 Hz), 3.619 (3H, s), 3.730 (1H, d, J=11.4 Hz), 3.872 (1H, d, J=11.4 Hz), 3.855 (3H, s), 3.855 (3H, s), 3.894 (3H, s), 4.406 (1H, t, J=6.4 Hz), 4.560 (1H, d, J=14.0 Hz), 6.299 (1H, s), 6.642 (1H, dd, J=2.2 Hz), 6.96-7.48 (6H, m), 7.712 (1H, d, J=2.6 Hz), 7.786 (1H, br).

Elemental analysis (C₃₅H₄₃N₂O₉Cl) Cal'd: C, 64.54; H, 6.13; N, 3.96. Found: C, 64.32; H, 5.94; N, 3.84.

(4) A mixture of methyl 3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-5,6,7,8-tetrahydro-1-naphthoate obtained in Example 112-(3) (1 g, 1.41 mmol), 1 N aqueous sodium hydroxide solution (3 ml) and ethanol (10 ml) was stirred at 60° C. for 1 hour. This was diluted with water (50 ml) and, after acidification, extracted with ethyl acetate (100 ml). The extract was washed with saturated saline, dried with sodium sulfate and concentrated under reduced pressure. The residue was purified by recrystallization from ethyl acetate-hexane (2:1) to obtain 3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-5,6,7,8-tetrahydro-1-naphthoic acid (0.42 g, 0.645 mmol, 46%) as a colorless powder.

Melting point 178-179° C.

[α]_{D}²²−125.4° (c=0.14, MeOH).

IR νₘₐₓ (KBr) cm⁻¹: 3600-2400 (br, COOH, OH), 1660 (C═O).

¹H-NMR (CDCl₃) δ: 0.652 (3H, s), 1.046 (3H, s), 1.70-1.80 (4H, m), 2.72-2.89 (3H, m), 2.97-3.08 (3H, m), 3.189 (1H, d, J=11.8 Hz), 3.386 (1H, d, J=14.2 Hz), 3.608 (3H, s), 3.634 (1H, d, J=11.8 Hz), 3.885 (3H, s), 4.42-4.52 (2H, m), 6.192 (1H, s), 6.617 (1H, s) 6.96-7.34 (5H, m), 7.601 (1H, s), 7.742 (1H, s), 7.95-8.04 (1H, br).

Elemental analysis (C₃₅H₃₉N₂O₈Cl.H₂O) Cal'd: C, 62.82; H, 6.18; N, 4.19. Found: C, 62.55; H, 6.00; N, 3.98.

### EXAMPLE 113

4-[[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]butanoic acid

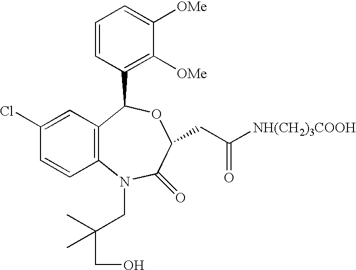

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (2.0 g, 4.18 mmol) and methyl 4-aminobutanoate hydrochloride (0.71 g, 4.60 mmol) in N,N-dimethylformamide (20 ml) were added diethyl cyanophosphate (0.82 g, 5.02 mmol) and then triethylamine (1.1 g, 10.5 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by column chromatography (eluent:ethyl acetate) and recrystallized from ethyl acetate-hexane (1:1) to obtain methyl 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]butanoate (1.59 g, 2.76 mmol, 66%) as a colorless powder.

Melting point 78-80° C.

[α]_{D}²²−202.4° (c=0.15, MeOH).

IR νₘₐₓ (KBr) cm⁻¹: 3600-3200 (br, OH, NH), 1738, 1651 (C═O).

¹H-NMR (CDCl₃) δ: 0.637 (3H, s), 1.048 (3H, s), 1.839 (2H, quintet, J=7.2 Hz), 2.357 (2H, t, J=7.2 Hz), 2.630 (1H, dd, J=5.8, 14.2 Hz), 2.829 (1H, dd, J=7.4, 14.2 Hz), 3.139 (1H, t, J=10.8 Hz), 3.23-3.34 (2H, m), 3.376 (1H, d, J=14.6 Hz), 3.58-3.67 (1H, br), 3.608 (3H, s), 3.674 (3H, s), 3.892 (3H, s), 4.14-4.22 (1H, br), 4.403 (1H, dd, J=5.8, 7.4 Hz), 4.459 (1H, d, J=14.6 Hz), 5.96-6.03 (1H, br), 6.153 (1H, s), 6.607 (1H, d, J=11.4 Hz), 6.97-7.40 (5H, m).

Elemental analysis (C₂₉H₃₇N₂O₈Cl.0.5H₂O) Cal'd: C, 59.43; H, 6.54; N, 4.78. Found: C, 59.58; H, 6.51; N, 4.54.

(2) A mixture of methyl 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]butanoate obtained in Example 113-(1) (1.49 g, 2.58 mmol), 1 N aqueous sodium hydroxide solution (6 ml) and ethanol (10 ml) was stirred at 60° C. for 30 minutes. This was diluted with water (100 ml) and, after acidification, extracted with ethyl acetate (100 ml). The extract was washed with saturated saline, dried with sodium sulfate and concentrated under reduced pressure. The residue was purified by recrystallization from ethyl acetate-hexane (1:1) to obtain 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]butanoic acid (1.1 g, 1.95 mmol, 76%) as colorless prisms.

Melting point 111-113° C.

[α]_{D}²²−203.1° (c=0.11, MeOH).

IR νₘₐₓ (KBr) cm⁻¹: 3600-2200 (br, COOH, OH, NH), 1716, 1651 (C═O).

¹H-NMR (CDCl₃) δ: 0.643 (3H, s), 1.038 (3H, s), 1.835 (2H, quintet, J=6.8 Hz), 2.372 (2H, t, J=6.8 Hz), 2.647 (1H, dd, J=5.4, 14.2 Hz), 2.841 (1H, dd, J=7.6, 14.2 Hz), 3.158 (1H, t, J=10.8 Hz), 3.25-3.33 (2H, m), 3.387 (1H, d, J=14.6 Hz), 3.601 (3H, s), 3.604 (1H, d, J=10.8 Hz), 3.886 (3H, s), 4.37-4.48 (2H, m). 6.146 (1H, s), 6.22-6.30 (1H, br), 6.610 (1H, d, J=1.4 Hz), 6.96-7.36 (5H, m).

Elemental analysis (C₂₈H₃₅N₂O₈Cl.0.5H₂O) Cal'd: C, 58.79; H, 6.34; N, 4.90. Found: C, 58.94; H, 6.53; N, 4.52.

### EXAMPLE 114

4-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]butanoic acid

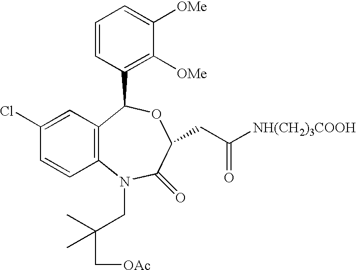

To a mixture of 4-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]butanoic acid obtained in Example 113-(2) (0.10 g, 0.178 mmol), pyridine (63 mg, 0.799 mmol) and ethyl acetate (2 ml) was added acetyl chloride (49 mg, 0.622 mmol). The mixture was stirred at room temperature for 1 hour and, after addition of water (2 ml), it was further stirred at room temperature for 2 hours. The organic layer was separated, washed with 1 N hydrochloric acid and saturated saline, dried by sodium sulfate and concentrated under reduced pressure to obtain 4-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]butanoic acid (0.44 g, 0.608 mmol, 86%) as a colorless amorphous powder.

[α]_{D}²²−196.1° (c=0.18, MeOH).

IR νₘₐₓ (KBr) cm⁻¹: 3400-2200 (br, COOH, NH), 1732, 1676 (C═O).

¹H-NMR (CDCl₃) δ: 0.943 (3H, s), 1.000 (3H, s), 1.830 (2H, quintet, J=6.8 Hz), 2.027 (3H, s), 2.363 (2H, t, J=6.8 Hz), 2.651 (1H, dd, J=5.6, 14.4 Hz), 2.834 (1H, dd, J=7.2, 14.4 Hz), 3.301 (2H, q, J=6.8 Hz), 3.532 (1H, t, J=14.4 Hz), 3.606 (3H, s), 3.720 (1H, d, J=11.0 Hz), 3.863 (1H, d, J=11.0 Hz), 3.888 (3H, s), 4.382 (1H, dd, J=5.6, 7.2 Hz), 4.532 (1H, t, J=14.4 Hz), 6.247 (1H, s), 6.26-6.36 (1H, br), 6.635 (1H, d, J=1.8 Hz), 6.96-7.34 (5H, m).

Elemental analysis (C₃₀H₃₇N₂O₉Cl) Cal'd: C, 59.55; H, 6.16; N, 4.63. Found: C, 59.45; H, 6.30; N, 4.38.

### EXAMPLE 115

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]pentanoic acid

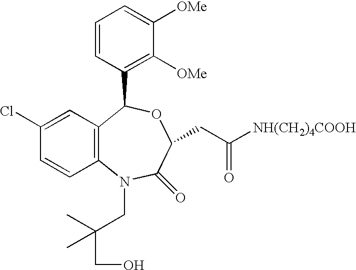

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (2.0 g, 4.18 mmol) and methyl 5-aminopentanoate hydrochloride (0.77 g, 4.60 mmol) in N,N-dimethylformamide (20 ml) were added diethyl cyanophosphate (0.82 g, 5.02 mmol) and then triethylamine (1.1 g, 10.5 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by recrystallization from ethyl acetate-hexane (1:1) to obtain methyl 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]pentanoate (2.57 g, 4.35 mmol, quant) as colorless prisms.

Melting point 84-85° C.

[α]_{D}²²−190.6° (c=0.13, MeOH).

IR νₘₐₓ (KBr) cm⁻¹: 3600-3200 (br, OH, NH), 1738, 1660 (C═O).

¹H-NMR (CDCl₃) δ: 0.637 (3H, s), 1.046 (3H, s), 1.45-1.68 (4H, m), 2.337 (2H, t, J=7.0 Hz), 2.627 (1H, dd, J=5.6, 14.4 Hz), 2.840 (1H, dd, J=7.4, 14.4 Hz), 3.139 (1H, t, J=11.2 Hz), 3.237 (2H, q, J=6.2 Hz), 3.379 (1H, d, J=14.2 Hz), 3.606 (3H, s), 3.610 (1H, dd, J=4.4, 11.2 Hz), 3.672 (3H, s), 3.892 (3H, s), 4.196 (1H, dd, J=4.4, 11.2 Hz), 4.401 (1H, dd, J=5.6, 7.4 Hz), 4.459 (1H, d, J=14.2 Hz), 5.88-5.94 (1H, br), 6.151 (1H, s), 6.601 (1H, s), 6.96-7.36 (5H, m).

Elemental analysis (C₃₀H₃₉N₂O₈Cl.H₂O) Cal'd: C, 59.16; H, 6.78; N, 4.60. Found: C, 59.05; H, 6.64; N, 4.29.

(2) A mixture of methyl 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]pentanoate obtained in Example 115-(1) (2.3 g, 3.89 mmol), 1 N aqueous sodium hydroxide solution (8 ml) and ethanol (20 ml) was stirred at 60° C. for 30 minutes. This was diluted with water (100 ml) and, after acidification, extracted twice with ethyl acetate (each 100 ml). The extract was washed with saturated saline, dried with sodium sulfate and concentrated under reduced pressure to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]pentanoic acid (2.1 g, 3.69 mmol, 95%) as a colorless amorphous powder.

[α]_{D}²²−191.3° (c=0.24, MeOH).

IR νₘₐₓ (KBr) cm⁻¹: 3600-2200 (br, COOH, OH, NH), 1714, 1651 (C═O).

¹H-NMR (CDCl₃) δ: 0.641 (3H, s), 1.035 (3H, s), 1.45-1.75 (4H, m), 2.359 (2H, t, J=7.0 Hz), 2.647 (1H, dd, J=5.6, 14.4 Hz), 2.848 (1H, dd, J=7.6, 14.4 Hz), 3.155 (1H, t, J=12.0 Hz), 3.23-3.28 (2H, m), 3.382 (1H, d, J=14.4 Hz), 3.601 (3H, s), 3.603 (1H, d, J=12.0 Hz), 3.888 (3H, s), 4.400 (1H, dd, J=5.6, 7.6 Hz), 4.446 (1H, d, J=14.4 Hz), 6.02-6.14 (1H, br), 6.143 (1H, s), 6.603 (1H, s), 6.96-7.36 (5H, m).

Elemental analysis (C₂₉H₃₇N₂O₈Cl.H₂O) Cal'd: C, 58.53; H, 6.61; N, 4.71. Found: C, 58.77; H, 6.71; N, 4.36.

### EXAMPLE 116

5-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]pentanoic acid

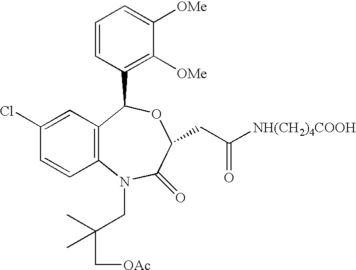

To a mixture of 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]pentanoic acid obtained in Example 115-(2) (0.43 g, 0.745 mmol), pyridine (0.27 g, 0.799 mmol) and ethyl acetate (5 ml) was added acetyl chloride (0.20 g, 2.61 mmol). The mixture was stirred at room temperature for 1 hour and, after addition of water (4 ml), it was further stirred at 60° C. for 3 hours. The organic layer was separated, washed with 1 N hydrochloric acid and saturated saline, dried by sodium sulfate and concentrated under reduced pressure to obtain 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]pentanoic acid (0.37 g, 0.598 mmol, 80%) as a colorless amorphous powder.

[α]_{D}²²−183.0° (c=0.17, MeOH).

IR νₘₐₓ (KBr) cm⁻¹: 3600-2400 (br, COOH, NH), 1732, 1678 (C═O).

¹H-NMR (CDCl₃) δ: 0.936 (3H, s), 1.006 (3H, s), 1.45-1.75 (4H, m), 2.026 (3H, s), 2.354 (2H, t, J=7.0 Hz), 2.627 (1H, dd, J=5.8, 14.2 Hz), 2.838 (1H, dd, J=7.6, 14.2 Hz), 3.242 (2H, q, J=6.2 Hz), 3.531 (1H, t, J=14.0 Hz), 3.605 (3H, s), 3.717 (1H, dd, J=11.0 Hz), 3.863 (1H, dd, J=11.0 Hz), 3.887 (3H, s), 4.383 (1H, dd, J=5.8, 7.6 Hz), 4.527 (1H, d, J=14.0 Hz), 6.12-6.22 (1H, br) 6.244 (1H, s), 6.625 (1H, s), 6.96-7.33 (5H, m).

Elemental analysis (C₃₁H₃₉N₂O₉Cl) Cal'd: C, 60.14; H, 6.35; N, 4.52. Found: C, 59.94; H, 6.67; N, 4.13.

### EXAMPLE 117

6-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]hexanoic acid

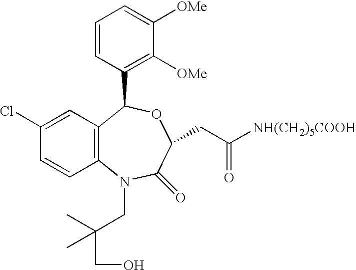

(1) To a solution of (3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid (2.0 g, 4.18 mmol) and methyl 6-aminohexanoate hydrochloride (0.84 g, 4.60 mmol) in N,N-dimethylformamide (20 ml) were added diethyl cyanophosphate (0.82 g, 5.02 mmol) and then triethylamine (1.1 g, 10.5 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was purified by column chromatography [eluent:hexane-ethyl acetate (1:6)] and recrystallized from ethyl acetate-hexane (1:1) to obtain methyl 6-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]hexanoate (2.30 g, 3.80 mmol, 91%) as colorless prisms.

Melting point 131-132° C.

[α]_{D}²²−200.7° (c=0.26, MeOH).

IR νₘₐₓ (KBr) cm⁻¹: 3500-3200 (br, OH, NH), 1738, 1658 C═O).

¹H-NMR (CDCl₃) δ: 0.637 (3H, s), 1.048 (3H, s), 1.26-1.72 (6H, m), 2.313 (2H, t, J=7.5 Hz), 2.623 (1H, dd, J=6.0, 14.4 Hz), 2.825 (1H, dd, J=7.4, 14.4 Hz), 3.11-3.29 (3H, m), 3.608 (3H, s), 3.611 (1H, d, J=11.8 Hz), 3.671 (3H, s), 3.894 (3H, s), 4.1-4.3 (1H, br), 4.406 (1H, dd, J=6.0, 7.4 Hz), 4.457 (1H, d, J=14.6 Hz), 5.82-5.88 (1H, br), 6.153 (1H, s), 6.605 (1H, d, J=1.8 Hz), 6.97-7.36 (5H, m).

Elemental analysis (C₃₁H₄₁N₂O₈Cl) Cal'd: C, 61.53; H, 6.83; N, 4.63. Found: C, 61.32; H, 7.01; N, 4.40.

(2) A mixture of methyl 6-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]hexanoate obtained in Example 117-(1) (2.2 g, 3.64 mmol), 1 N aqueous sodium hydroxide solution (8 ml) and ethanol (20 ml) was stirred at 60° C. for 30 minutes. This was diluted with water (50 ml) and, after acidification, extracted twice with ethyl acetate (each 100 ml). The extract was washed with saturated saline, dried with sodium sulfate and concentrated under reduced pressure. The residue was purified by recrystallization from ethyl acetate-hexane (2:1) to obtain 6-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]hexanoic acid (2.1 g, 3.49 mmol, 96%) as a colorless powder.

Melting point 96-98° C.

[α]_{D}²²−182.4° (c=0.19, MeOH).

IR νₘₐₓ (KBr) cm⁻¹: 3600-2200 (br, COOH, OH, NH), 1720, 1651 (C═O).

¹H-NMR (CDCl₃) δ: 0.645 (3H, s), 1.042 (3H, s), 1.36-1.72 (6H, m), 2.342 (2H, t, J=7.0 Hz), 2.629 (1H, dd, J=5.8, 14.2 Hz), 2.822 (1H, dd, J=7.6, 14.2 Hz), 3.168 (1H, t, J=12.2 Hz), 3.23-3.30 (2H, m), 3.382 (1H, d, J=14.4 Hz), 3.601 (3H, s), 3.607 (1H, dd, J=12.2 Hz), 3.892 (3H, s), 4.400 (1H, dd, J=5.6, 7.6 Hz), 4.446 (1H, d, J=14.4 Hz), 6.02-6.14 (1H, br), 6.143 (1H, s), 6.603 (1H, s), 6.96-7.36 (5H, m).

Elemental analysis (C₃₀H₃₉N₂O₈C.AcOEt.0.5H₂O) Cal'd: C, 59.34; H, 7.03; N, 4.07. Found: C, 59.37; H, 6.81; N, 4.03.

### EXAMPLE 118

6-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]hexanoic acid

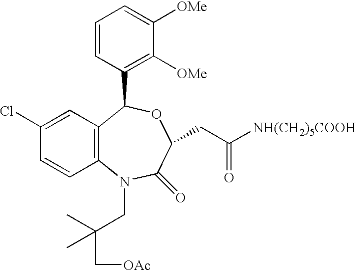

To a mixture of 6-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]hexanoic acid obtained in Example 117-(2) (0.3 g, 0.508 mmol), pyridine (0.18 g, 2.28 mmol) and ethyl acetate (5 ml) was added acetyl chloride (0.14 g, 1.78 mmol). The mixture was stirred at room temperature for 1 hour and, after addition of water (4 ml), it was further stirred at 60° C. for 3 hours. The organic layer was separated, washed with 1 N hydrochloric acid and saturated saline, dried by sodium sulfate and concentrated under reduced pressure to obtain 6-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]hexanoic acid (0.23 g, 0.363 mmol, 72%) as a colorless amorphous powder.

[α]_{D}²²−194.4° (c=0.22, MeOH).

IR νₘₐₓ (KBr) cm⁻: 3600-2400 (br, COOH, NH), 1732, 1680 (C═O).

¹H-NMR (CDCl₃) δ: 0.943 (3H, s), 1.007 (3H, s), 1.26-1.72 (6H, m), 2.027 (3H, s), 2.330 (2H, t, J=7.0 Hz), 2.628 (1H, dd, J=5.8, 14.2 Hz), 2.816 (1H, dd, J=7.2, 14.2 Hz), 3.226 (2H, q, J=6.6 Hz), 3.531 (1H, t, J=14.0 Hz), 3.606 (3H, s), 3.725 (1H, dd, J=11.4 Hz), 3.870 (1H, d, J=111.4 Hz), 3.888 (3H, s), 4.384 (1H, dd, J=5.8, 7.2 Hz), 4.536 (1H, d, J=14.0 Hz), 6.02-6.08 (1H, br) 6.251 (1H, s), 6.627 (1H, d, J=1.4 Hz), 6.96-7.37 (5H, m).

Elemental analysis (C₃₂H₄₁N₂O₉Cl) Cal'd: C, 60.71; H, 6.53; N, 4.42. Found: C, 60.36; H, 6.66; N, 4.05.

### EXAMPLE 119

2-[2-[[[(3S,5R)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylic acid

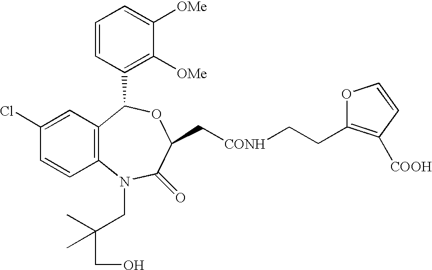

(1) To a solution of (3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-acetic acid synthesized by the same manner as that disclosed in JP 09-136880 A, Example 11-(4) (0.8 g, 1.67 mmol) and methyl 2-(2-aminoethyl)furan-3-carboxylate hydrochloride (0.34 g, 1.76 mmol) in N,N-dimethylformamide (8 ml) were added diethyl cyanophosphate (0.30 g, 1.84 mmol) and then triethylamine (0.42 g, 4.18 mmol). The mixture was stirred at room temperature for 30 minutes. This was diluted with ethyl acetate (100 ml), washed with water, 5% aqueous potassium hydrogen sulfate solution, saturated aqueous sodium hydrogen carbonate solution and saturated saline, dried with sodium sulfate, and then concentrated under reduced pressure. The residue was recrystallized from ethyl acetate-hexane (1:1) to obtain methyl 2-[2-[[[(3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylate (1.1 g, 1.75 mmol, quant) as a colorless powder.

Melting point 82-85° C.

[α]_{D}²²+73.7° (c=0.12, MeOH).

IR νₘₐₓ (KBr) cm⁻¹: 3600-3200 (br, OH, NH), 1714, 1658 (C═O).

¹H-NMR (CDCl₃) δ: 0.637 (3H, s), 1.046 (3H, s), 2.585 (1H, dd, J=5.6, 14.4 Hz), 2.829 (1H, dd, J=7.8, 14.4 Hz), 3.10-3.23 (3H, m), 3.344 (1H, d, J=14.2 Hz), 3.51-3.63 (3H, m), 3.597 (3H, s), 3.837 (3H, s), 3.889 (3H, s), 4.380 (1H, d, J=5.6, 7.8 Hz), 4.409 (1H, d, J=14.2 Hz), 6.127 (1H, s), 6.30-6.38 (1H, br), 6.594 (1H, d, J=2.0 Hz), 6.656 (1H, d, J=2.0 Hz), 6.96-7.35 (6H, m).

Elemental analysis (C₃₂H₃₇N₂O₈Cl.0.8H₂O) Cal'd: C, 59.73; H, 6.05; N, 4.35. Found: C, 59.72; H, 6.13; N, 4.25.

(2) A mixture of methyl 2-[2-[[[(3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylate obtained in Example 119-(1) (0.98 g, 1.56 mmol), 1 N aqueous sodium hydroxide solution (4 ml) and ethanol (10 ml) was stirred at 60° C. for 30 minutes. This was diluted with water (100 ml) and, after acidification, extracted twice with ethyl acetate (each 100 ml). The extract was washed with saturated saline, dried with sodium sulfate and concentrated under reduced pressure. The residue was purified by recrystallization from ethyl acetate-hexane (2:1) to obtain 2-[2-[[[(3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylate (0.47 g, 0.764 mmol, 49%) as a colorless powder.

Melting point 123-126° C.

[α]_{D}²²+190.4° (c=0.26, MeOH).

IR νₘₐₓ (KBr) cm⁻¹: 3600-2400 (br, COOH, OH, NH), 1660 (C═O).

¹H-NMR (CDCl₃) δ: 0.628 (3H, s), 1.042 (3H, s), 2.590 (1H, dd, J=5.6, 14.8 Hz), 2.853 (1H, dd, J=8.0, 14.8 Hz), 3.13-3.25 (3H, m), 3.351 (1H, d, J=14.4 Hz), 3.52-3.63 (3H, m), 3.585 (3H, s), 3.879 (3H, s), 4.375 (1H, dd, J=5.6, 8.0 Hz), 4.413 (1H, d, J=14.4 Hz), 6.118 (1H, s), 6.42-6.54 (1H, br), 6.581 (1H, s), 6.690 (1H, d, J=2.2 Hz), 6.94-7.33 (6H, m).

Elemental analysis (C31H₃₅N₂O₉Cl.H₂O) Cal'd: C, 58.81; H, 5.89; N, 4.42. Found: C, 58.82; H, 5.84; N, 4.45.

### EXAMPLE 120

3-[3-[[(3S,5R)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic Acid

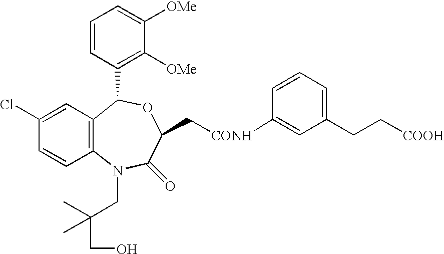

(1) To a mixture of (3S,5R)-7-chloro-5-(2,3-dimethoxyphenyl)-1-(3-acetoxy-2,2-dimethylpropyl)-2-oxo-1,2,3,5-tetrahydro-4,1-benzoxazepin-3-acetic acid (3.0 g, 6.28 mmol), pyridine (2.2 g, 28.2 mmol) and ethyl acetate (30 ml) was added thionyl chloride (1.7 g, 22.0 mmol). After stirring at room temperature for 1 hour, water (25 ml) was added to the mixture and the mixture was further stirred at room temperature for 3 hours. The organic layer was separated, washed with 1 N hydrochloric acid and saturated saline, dried with sodium sulfate and concentrated under reduced pressure. The residue was purified by recrystallization from ethyl acetate-hexane (1:2) to obtain (3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid (2.9 g, 5.54 mmol, 88%) as colorless prisms.

Melting point 185-187° C.

[α]_{D}²²+224.4° (c=0.23, MeOH).

IR νₘₐₓ (KBr) cm⁻¹: 3600-2400 (br, COOH, NH), 1738, 1682 (C═O).

¹H-NMR (CDCl₃) δ: 0.943 (3H, s), 1.024 (3H, s), 2.029 (3H, s), 2.821 (1H, dd, J=5.4, 16.8 Hz), 3.084 (1H, dd, J=7.8, 16.8 Hz), 3.556 (1H, d, J=14.4 Hz), 3.616 (3H, s), 3.733 (1H, d, J=11.0 Hz), 3.856 (1H, d, J=11.0 Hz), 3.890 (3H, s), 4.331 (1H, dd, J=5.4, 7.8 Hz), 4.580 (1H, d, J=14.4 Hz), 6.259 (1H, s), 6.645 (1H, s), 6.96-7.35 (5H, m).

Elemental analysis (C₂₆H₃₀NO₈Cl) Cal'd: C, 60.06; H, 5.823; N, 2.69. Found: C, 60.06; H, 5.95; N, 2.45.

(2) To a solution of (3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 120-(1) (1.0 g, 1.92 mmol) and N,N-dimethylformamide (0.02 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (10 ml) and added to a mixture of ethyl 3-(3-aminophenyl)propionate (0.46 g, 2.01 mmol), triethylamine (0.6 g, 5.94 mmol) and tetrahydrofuran (10 ml). After stirring at room temperature for 30 minutes, water was added and tetrahydrofuran was distilled off. The residue was diluted with ethyl acetate (100 ml). This was washed with 1 N hydrochloric acid and saturated saline, dried with sodium sulfate and concentrated under reduced pressure. The residue was purified by silica gel column chromatography [eluent:ethyl acetate-hexane (1:1)] to obtain ethyl 3-[3-[[(3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate (0.97 g, 1.40 mmol, 73%) as a colorless amorphous powder.

[α]_{D}²²+36.7° (c=0.21, MeOH).

IR νₘₐₓ (KBr) cm⁻¹: 3333(NH), 1732, 1682 (C═O).

¹H-NMR (CDCl₃) δ: 0.958 (3H, s), 1.024 (3H, s), 1.236 (3H, t, J=7.4 Hz), 2.024 (3H, s), 2.603 (2H, t, J=8.0 Hz), 2.812 (1H, dd, J=5.8, 14.0 Hz), 2.927 (2H, t, J=8.0 Hz), 2.996 (1H, dd, J=7.4, 14.0 Hz), 3.538 (1H, d, J=14.2 Hz), 3.619 (3H, s), 3.731 (1H, d, J=11.0 Hz), 3.872 (1H, d, J=11.0 Hz), 3.894 (3H, s), 4.128 (2H, q, J=7.4 Hz), 4.403 (1H, dd, J=5.8, 7.4 Hz), 4.564 (1H, d, J=14.2 Hz), 6.301 (1H, s), 6.644 (1H, d, J=2.0 Hz), 6.93-7.40 (9H, m), 7.801 (1H, brs).

Elemental analysis (C₃₇H₄₃N₂O₉Cl) Cal'd: C, 63.92; H, 6.23; N, 4.03. Found: C, 63.80; H, 6.27; N, 4.04.

(3) A mixture of ethyl 3-[3-[[(3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminophenyl]propionate obtained in Example 120-(2) (0.87 g, 1.25 mmol), 1 N aqueous sodium hydroxide solution (3 ml) and ethanol (8 ml) was stirred at 60° C. for 30 minutes. This was diluted with water (50 ml) and, after acidification, extracted twice with ethyl acetate (each 50 ml). The extract was washed with saturated saline, dried with sodium sulfate and concentrated under reduced pressure. The residue was purified by recrystallization from ethanol-hexane (1:2) to obtain 3-[3-[[[(3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid (0.58 g, 0.929 mmol, 74%) as colorless needles.

Melting point 137-139° C.

[α]_{D}²²+45.1° (c=0.13, MeOH).

IR νₘₐₓ (KBr) cm⁻¹: 3600-2400 (br, COOH, NH, OH), 1714, 1682, 1653 (C═O).

¹H-NMR (CDCl₃) δ: 0.654 (3H, s), 1.048 (3H, s), 2.645 (2H, t, J=7.3 Hz), 2.829 (1H, dd, J=5.8, 14.2 Hz), 2.929 (2H, t, J=7.3 Hz), 3.011 (1H, dd, J=7.2, 14.2 Hz), 3.186 (1H, d, J=12.0 Hz), 3.388 (1H, d, J=14.2 Hz), 3.608 (3H, s), 3.624 (1H, d, J=12.0 Hz), 3.890 (3H, s), 4.433 (1H, dd, J=5.8, 7.2 Hz), 4.474 (1H, d, J=14.2 Hz), 6.183 (1H, s), 6.625 (1H, d, J=1.8 Hz), 6.93-7.38 (9H, m), 7.973 (1H, brs).

Elemental analysis (C₃₃H₃₇N₂O₈Cl.0.5H₂O) Cal'd: C, 62.51; H, 6.04; N, 4.42. Found: C, 62.54; H, 5.97; N, 4.41.

### EXAMPLE 121

3-[3-[[[(3S,5R)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic Acid

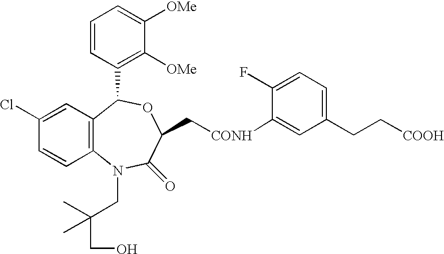

(1) To a solution of (3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid obtained in Example 120-(1) (1.0 g, 1.92 mmol) and N,N-dimethylformamide (0.02 ml) in tetrahydrofuran (10 ml) was added thionyl chloride (0.7 g, 5.88 mmol) at room temperature. After stirring for 1 hour, the mixture was concentrated under reduced pressure. The residue was dissolved in tetrahydrofuran (10 ml) and added to a mixture of ethyl 3-(3-amino-4-fluorophenyl)propionate (0.43 g, 2.01 mmol), 4-(N,N-dimethylamino)pyridine (0.28 g, 2.30 mmol) and tetrahydrofuran (10 ml). After stirring at room temperature for 30 minutes, water was added and tetrahydrofuran was distilled off. The residue was diluted with ethyl acetate (50 ml). This was washed with 1 N hydrochloric acid and saturated saline, dried with sodium sulfate and concentrated under reduced pressure. The residue was purified by silica gel column chromatography [eluent:ethyl acetate-hexane (1:2)] to obtain ethyl 3-[3-[[[(3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionate (0.71 g, 0.996 mmol, 52%) as a colorless amorphous powder.

[α]_{D}²²+29.9° (c=0.25, MeOH).

IR νₘₐₓ (KBr) cm⁻¹: 3331(NH), 1732, 1682 (C═O).

¹H-NMR (CDCl₃) δ: 0.955 (3H, s), 1.022 (3H, s), 1.226 (3H, t, J=7.4 Hz), 2.024 (3H, s), 2.577 (2H, t, J=7.9 Hz), 2.849 (1H, dd, J=5.4, 14.6 Hz), 2.894 (2H, t, J=7.9 Hz), 3.058 (1H, dd, J=7.4, 14.6 Hz), 3.546 (1H, d, J=14.2 Hz), 3.618 (3H, s), 3.721 (1H, d, J=11.0 Hz), 3.869 (1H, d, J=11.0 Hz), 3.889 (3H, s), 4.112 (2H, q, J=7.4 Hz), 4.405 (1H, dd, J=5.4, 7.4 Hz), 4.581 (1H, d, J=14.2 Hz), 6.294 (1H, s), 6.646 (1H, s), 6.83-7.34 (7H, m), 7.986 (1H, brs), 8.11-8.15 (1H, m).

Elemental analysis (C₃₇H₄₂N₂O₉ClF) Cal'd: C, 62.31; H, 5.94; N, 3.93. Found: C, 62.13; H, 6.07; N, 3.81.

(2) A mixture of ethyl 3-[3-[[[(3S,5R)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionate obtained in Example 121-(1) (0.61 g, 0.855 mmol), 1 N aqueous sodium hydroxide solution (2 ml) and ethanol (6 ml) was stirred at 60° C. for 30 minutes. This was diluted with water (50 ml) and, after acidification, extracted with ethyl acetate (100 ml). The extract was washed with saturated saline, dried with sodium sulfate and concentrated under reduced pressure. The residue was purified by recrystallization from ethanol-hexane (1:2) to obtain 3-[3-[[[(3S,5R)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid (0.31 g, 0.482 mmol, 56%) as colorless needles.

Melting point 151-153° C.

[α]_{D}²²+144.7° (c=0.16, MeOH).

IR νₘₐₓ (KBr) cm⁻¹: 3600-2400 (br, COOH, NH, OH), 1714, 1695, 1682, 1660, 1651 (C═O).

¹H-NMR (CDCl₃) δ: 0.657 (3H, s), 1.053 (3H, s), 2.630 (2H, t, J=7.6 Hz), 2.861 (1H, dd, J=6.8, 15.0 Hz), 3.170 (1H, d, J=12.2 Hz), 3.401 (1H, d, J=14.2 Hz), 3.616 (3H, s), 3.617 (1H, d, J=12.2 Hz), 3.894 (3H, s), 4.431 (1H, dd, J=5.4, 6.8 Hz), 4.492 (1H, d, J=14.2 Hz), 6.195 (1H, s), 6.632 (1H, s), 6.88-7.42 (7H, m), 7.953 (1H, brs), 8.09-8.12 (1H, m).

Elemental analysis (C₃₃H₃₆N₂O₈ClF) Cal'd: C, 61.63; H, 5.64; N, 4.36. Found: C, 61.61; H, 5.75; N, 4.25.

### EXAMPLE 122

3-[4-Chloro-3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]propionic Acid

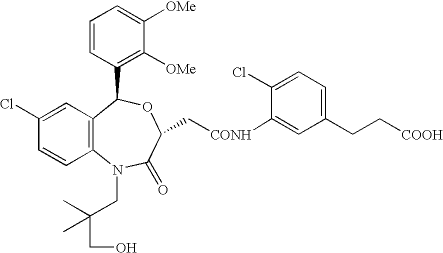

(1) A mixture of 3-(3-chloro-4-nitrophenyl)-2-propeonic acid (5 g, 22.0 mmol), potassium carbonate (4.3 g, 31.1 mmol), iodomethane (3.9 g, 27.2 mmol) and N,N-dimethylformamide (50 ml) was stirred at room temperature for 1 hour. This mixture was diluted with water and extracted with ethyl acetate (100 ml). The extract was washed with an aqueous saturated sodium chloride solution, dried with anhydrous sodium sulfate and concentrated under reduced pressure. The residue was recrystallized from ethyl acetate-hexane (1:2) to obtain methyl 3-(4-chloro-3-nitrophenyl)-2-propenoate (4.6 g, 18.4 mmol, 84%) as pale yellow needles.

m.p. 107° C.

(2) 10% Palladium carbon (0.5 g) was added to a solution of methyl 3-(4-chloro-3-nitrophenyl)-2-propenoate (4.6 g, 18.4 mmol) obtained in Example 122-(1) in ethyl acetate (100 ml) and subjected to normal pressure catalytic reduction at room temperature for 6 hours. The catalyst was filtered to remove, and the filtrate was concentrated under reduced pressure. The residue was dissolved in ethyl acetate (50 ml), a 4N hydrogen chloride-ethyl acetate solution (7 ml) was added and concentrated under reduced pressure. The residue was washed with ethyl acetate-hexane (1:1) to obtain methyl 3-(3-amino-4-chlorophenyl)propionate (4.3 g, 17.2 mmol, 93%) as a colorless powder.

m.p. 160-163° C. (dec).

IR νₘₐₓ (KBr) cm⁻¹: 3200-2200 (br, NH₃⁺), 1734 (C═O).

¹H-NMR (D₂O) δ: 2.402 (2H, t, J=7.0 Hz), 2.631 (2H, t, J=7.0 Hz), 3.314 (3H, s), 6.95-7.11 (2H, m), 7.195 (1H, d, J=8.0 Hz).

(3) Thionyl chloride (1.4 g, 11.8 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (2.0 g, 3.84 mmol) obtained in Example 1-(1), N,N-dimethylformamide (0.04 ml) and tetrahydrofuran (10 ml) at room temperature, followed by stirring for 1 hour. The residue obtained by concentration under reduced pressure was dissolved in tetrahydrofuran (20 ml). This solution was added to a mixture of methyl 3-(3-amino-4-chlorophenyl)propionate (1.0 g, 4.02 mmol) obtained in Example 122-(2), triethylamine (1.0 g, 10.1 mmol) and tetrahydrofuran (20 ml). The mixture was stirred at room temperature for 30 minutes and diluted with ethyl acetate (100 ml). This was washed with 1N hydrochloric acid, an aqueous saturated sodium bicarbonate solution and an aqueous saturated sodium chloride solution, dried with anhydrous sodium sulfate and concentrated under reduced pressure. The residue was purified by silica gel column chromatography [eluent:hexane-ethyl acetate (1:l)] to obtain methyl 3-[4-chloro-3-[[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]amino]phenyl]propionate (1.56 g, 2.18 mmol, 57%) as a colorless amorphous powder.

[α]_{D}²²−148.7° (c=0.18, MeOH). IR νₘₐₓ (KBr) cm⁻: 3333 (NH), 1738, 1682 (C═O).

¹H-NMR (CDCl₃) δ: 0.952 (3H, s), 1.026 (3H, s), 2.024 (3H, s), 2.608 (2H, t, J=7.5 Hz), 2.846 (1H, dd, J=5.4, 14.2 Hz), 2.908 (2H, t, J=8.5 Hz), 3.097 (1H, dd, J=6.6, 14.2 Hz), 3.551 (1H, d, J=14.2 Hz), 3.621 (3H, s), 3.661 (3H, s), 3.719 (1H, d, J=11.4 Hz), 3.868 (1H, d, J=11.4 Hz), 3.894 (3H, s), 4.405 (1H, dd, J=5.4, 6.6 Hz), 4.590 (1H, d, J=14.2 Hz), 6.309 (1H, s), 6.652 (1H, s, 6.94-7.39 (8H, m), 8.231 (1H, s).

Elemental Analysis (C₃₆H₄₀N₂O₉Cl) Cal'd: C, 60.42; H, 5.63; N, 3.91. Found: C, 60.63; H, 5.80; N, 3.89.

(4) A mixture of methyl 3-[4-chloro-3-[[[(3R,5S)-7-chloro-5-(2,3-dimethoxyphenyl)-1-(3-acetoxy-2,2-dimethylpropyl)-2-oxo-1,2,3,5-tetrahydro-4,1-benzoxazepin-3-yl]acetyl]amino]phenyl]propionate (1.4 g, 1.96 mmol) obtained in Example 122-(3), a 1N aqueous sodium hydroxide (4.5 ml) and ethanol (15 ml) was stirred at 60° C. for 30 minutes. This was diluted with water (50 ml), acidified and extracted with ethyl acetate (100 ml). This was washed with an aqueous saturated sodium chloride solution, dried with anhydrous sodium sulfate and concentrated under reduced pressure. The residue was purified by recrystallization from ethanol-hexane (1:2) to obtain 3-[4-chloro-3-[[[(3R,5S)-7-chloro-5-(2,3-dimethoxyphenyl)-1-(3-hydroxy-2,2-dimehtylpropyl)-2-oxo-1,2,3,5-tetrahydro-4,1-benzoxazepin-3-yl]acetyl]amino]phenyl]propionic acid (1.0 g, 1.52 mmol, 77%) as a colorless powder.

[α]_{D}²²−162.9° (c=0.28, MeOH).

IR νₘₐₓ (KBr) cm⁻¹: 3600-2400 (br, COOH, NH, OH), 1711, 1660 (C═O).

¹H-NMR (CDCl₃) δ: 0.654 (3H, s), 1.048 (3H, s), 2.630 (2H, t, J=7.6 Hz), 2.847 (1H, dd, J=5.0, 15.0 Hz), 2.912 (2H, t, J=7.6 Hz), 3.110 (1H, dd, J=5.4, 15.0 Hz), 3.161 (1H, d, J=11.6 Hz), 3.395 (1H, d, J=14.2 Hz), 3.597 (1H, d, J=11.6 Hz), 3.606 (3H, s), 3.896 (3H, s), 4.421 (1H, dd, J=5.0, 5.4 Hz), 4.486 (1H, d, J=14.2 Hz), 6.205 (1H, s), 6.648 (1H, d, J=1.8 Hz), 6.86-7.42 (7H, m), 8.18-8.24 (2H, m).

Elemental Analysis (C₃₃H₃₆N₂O₈Cl₂) Cal'd: C, 60.09; H, 5.50; N, 4.25. Found: C, 60.48; H, 5.46; N, 4.04.

### EXAMPLE 123

3-[1-(3-Acetoxy-2,2-dimethylpropyl)-4-chloro-3-[[[(3R,5S)-7-chloro-5-(2,3-dimethoxyphenyl)-2-oxo-1,2,3,5-tetrahydro-4,1-benzoxazepin-3-yl]acetyl]amino]phenyl]propionic Acid

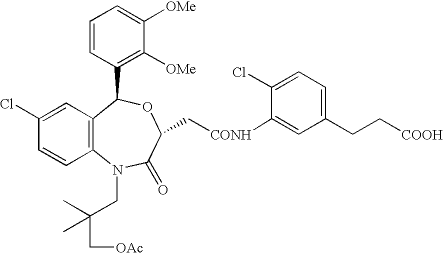

Acetyl chloride (83 mg, 1.06 mmol) was added to a mixture of 3-[4-chloro-3-[[[(3R,5S)-7-chloro-5-(2,3-dimethoxyphenyl)-1-(3-hydroxy-2,2-dimehtylpropyl)-2-oxo-1,2,3,5-tetrahydro-4,1-benzoxazepin-3-yl]acetyl]amino]phenyl]propionic acid (0.2 g, 0.303 mmol) obtained in Example 122-(4), pyridine (0.11 g, 1.36 mmol) and ethyl acetate (3 ml). The mixture was stirred at room temperature for 1 hour, and water (3 ml) was added to this mixture, followed by stirring at room temperature for 2 hours. The organic layer was separated, and washed with 1N hydrochloric acid and an aqueous saturated sodium chloride solution. This was dried with anhydrous sodium sulfate and concentrated under reduced pressure to obtain 3-[4-chloro-3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]propionic acid (0.12 g, 0.171 mmol, 56%) as a colorless amorphous powder.

[α]_{D}²²−149.0° (c=0.35, MeOH).

IR νₘₐₓ (KBr) cm⁻¹: 3600-2400 (br, COOH, NH), 1732, 1682 (C═O).

¹H-NMR (CDCl₃) δ: 0.952 (3H, s), 1.020 (3H, s), 2.022 (3H, s), 2.643 (2H, t, J=7.3 Hz), 2.851 (1H, dd, J=5.2, 14.2 Hz), 2.903 (2H, t, J=7.3 Hz), 3.099 (1H, dd, J=6.6, 14.2 Hz), 3.550 (1H, d, J=13.8 Hz), 3.621 (3H, s), 3.719 (1H, d, J=11.4 Hz), 3.868 (1H, d, J=11.4 Hz), 3.892 (3H, s), 4.410 (1H, dd, J=5.2, 6.6 Hz), 4.589 (1H, d, J=13.8 Hz), 6.309 (1H, s), 6.656 (1H, s), 6.85-7.38 (7H, m), 8.23-8.28 (2H, m).

### EXAMPLE 124

3-[5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-(3-phenylpropyloxy)phenyl]propionic Acid

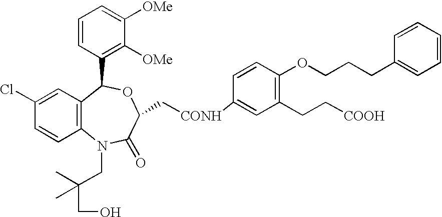

(1) A mixture of 2-hydroxy-nitrobenzaldehyde (2 g, 12.0 mmol), potassium carbonate (2.5 g, 18.0 mmol), 1-bromo-3-phenylpropane (2.6 g, 13.2 mmol) and N,N-dimethylformamide (20 ml) was stirred at 60° C. overnight. This mixture was diluted with water and extracted with ethyl acetate (100 ml). The extract was washed with an aqueous saturated sodium chloride solution, dried with anhydrous sodium sulfate and concentrated under reduced pressure. The residue was recrystallized from ethyl acetate-hexane (1:3) to obtain 2-(3-phenylpropyloxy)-5-nitrobenzaldehyde (2.6 g, 9.11 mmol, 76%) as colorless prisms.

m.p. 72-73° C.

IR νₘₐₓ (KBr) cm⁻¹: 1693 (C═O).

¹H-NMR (CDCl₃) δ: 2.258 (2H, quintet, J=7.4 Hz), 2.868 (2H, t, J=7.4 Hz), 4.216 (2H, t, J=7.4 Hz), 7.041 (1H, d, J=9.2 Hz), 7.18-7.36 (5H, m), 8.398 (1H, dd, J=2.6, 9.2 Hz), 8.704 (1H, d, J=2.6 Hz), 10.406 (1H, s).

Elemental Analysis (C₁₆H₁₅NO₄) Cal'd: C, 67.36; H, 5.30; N, 4.91. Found: C, 67.32; H, 5.15; N, 4.64.

(2) A solution of triethylphosphonoacetic acid (3.1 g, 8.83 mmol) in tetrahydrofuran (25 ml) was added to a mixture of 2-(3-phenylpropyloxy)-5-nitrobenzaldehyde (2.4 g, 8.41 mmol), sodium hydride (0.21 g, 8.83 mmol) and tetrahydrofuran (25 ml) at 0° C. The mixture was stirred at room temperature for 1 hour, and the reaction was quenched with a 5% aqueous potassium hydrogen sulfate solution. The mixture was diluted with ethyl acetate (100 ml), washed with an aqueous saturated sodium chloride solution, dried with anhydrous sodium sulfate and concentrated under reduced pressure. The residue was purified by recrystallization from ethyl acetate-hexane (1:2) to obtain ethyl 3-[2-(3-phenylpropyloxy)-5-nitrophenyl]-2-propenoate (2.43 g, 6.84 mmol, 81%) as colorless prisms.

m.p. 117-118° C.

IR νₘₐₓ (KBr) cm⁻¹: 1712, 1699 (C═O), 1635 (C═C).

¹H-NMR (CDCl₃) δ: 1.361 (3H, t, J=7.0 Hz), 2.17-2.31 (2H, m), 2.865 (2H, t, J=7.5 Hz), 4.139 (2H, t, J=7.5 Hz), 4.297 (2H, q, J=7.0 Hz), 6.667 (1H, d, J=16.0 Hz), 6.925 (1H, d, J=9.0 Hz), 7.18-7.35 (5H, m), 7.972 (1H, d, J=16.0 Hz), 8.208 (1H, dd, J=2.6, 9.0 Hz), 8.426 (1H, d, J=2.6 Hz).

Elemental Analysis (C₂₀H₂₁NO₅) Cal'd: C, 67.59; H, 5.96; N, 3.94. Found: C, 67.55; H, 6.01; N, 3.82.

(3) 10% Palladium carbon (0.2 g) and a 4N hydrogen chloride-ethyl acetate solution (2 ml) were added to a solution of ethyl 3-[2-(3-phenylpropyloxy)-5-nitrophenyl]-2-propenoate (2.3 g, 6.47 mmol) obtained in Example 124-(2) in ethanol (50 ml), which was subjected to normal pressure catalytic reduction at room temperature for 4 hours. The catalyst was filtered to remove, and the filtrate was concentrated under reduced pressure. The residue was washed with diethyl ether-hexane (1:1) to obtain ethyl 3-[5-amino-2-(3-phenylpropyloxy)phenyl]-2-propionate hydrochloride (2.1 g, 5.77 mmol, 89%) as a colorless powder.

m.p. 82-96° C.

IR νₘₐₓ (KBr) cm⁻¹: 3600-2400 (br, NH₃⁺), 1732 (C═O).

¹H-NMR (D₂O) δ: 0.75-0.95 (3H, m), 1.65-1.95 (2H, m), 2.24-2.78 (6H, m), 3.55-3.90 (4H, m), 6.90-7.08 (8H, m).

Elemental Analysis (C₂₀H₂₆NO₃Cl.0.2H₂O) Cal'd: C, 65.37; H, 7.24; N, 3.81. Found: C, 65.27; H, 7.06; N, 3.89.

(4) Thionyl chloride (0.7 g, 5.88 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1), N,N-dimethylformamide (0.02 ml) and tetrahydrofuran (10 ml) at room temperature, and the mixture was stirred. The residue obtained by concentration under reduced pressure was dissolved in tetrahydrofuran (10 ml). This solution was added to a mixture of ethyl 3-[5-amino-2-(3-phenylpropyloxy)phenyl]-2-propionate hydrochloride (0.73 g, 2.01 mmol) obtained in Example 124-(3), triethylamine (0.5 g, 5.05 mmol) and tetrahydrofuran (10 ml). The mixture was stirred at room temperature for 1 hour, and diluted with ethyl acetate (100 ml). This was washed with 1N hydrochloric acid, an aqueous saturated sodium bicarbonate solution and an aqueous saturated sodium chloride solution, dried with anhydrous sodium sulfate, and concentrated under reduced pressure. The residue was purified by silica gel chromatography [eluent:hexane-ethyl acetate (1:1)] to obtain ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-(3-phenylpropyloxy)phenyl]propionate (1.02 g, 1.23 mmol, 64%) as a brown oil.

IR νₘₐₓ (KBr) cm⁻¹: 3329 (NH), 1732, 1680 (C═O).

¹H-NMR (CDCl₃) δ: 0.958 (3H, s), 1.024 (3H, s), 1.232 (3H, t, J=7.4 Hz), 2.026 (3H, s), 2.05-2.18 (2H, m), 2.613 (2H, t, J=7.7 Hz), 2.78-3.02 (6H, m), 3.536 (1H, d, J=14.2 Hz), 3.619 (3H, s), 3.731 (1H, d, J=11.0 Hz), 3.86-3.98 (3H, m), 3.892 (3H, s), 4.129 (2H, q, J=7.4 Hz), 4.412 (1H, t, J=6.6 Hz), 4.562 (1H, d, J=14.2 Hz), 6.295 (1H, s), 6.48-7.35 (14H, m), 7.652 (1H, brs).

(5) A mixture of ethyl 3-[5-[[[(3R,5S)-1-3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-2-(3-phenylpropyloxy)phenyl]propionate (0.9 g, 1.09 mmol) obtained in Example 124-(4), a 1N aqueous sodium hydroxide solution (2.5 mmol) and ethanol (9 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate and concentrated under reduced pressure. The residue was purified by recrystallization from ethyl acetate-hexane (1:1) to obtain 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-(3-phenylpropyloxy)phenyl]propionic acid (0.35 g, 0.461 mmol, 42%) as a colorless powder.

m.p. 147-149° C.

[α]_{D}²²−93.2° (c=0.26, MeOH).

IR νₘₐₓ (KBr) cm⁻¹: 3600-2400 (br, COOH, NH, OH), 1726, 1651 (C═O).

¹H-NMR (CDCl₃) δ: 0.663 (3H, s), 1.039 (3H, s), 2.07-2.18 (2H, m), 2.610 (2H, t, J=7.6 Hz), 2.78-3.06 (6H, m), 3.148 (1H, d, J=11.4 Hz), 3.407 (1H, d, J=14.8 Hz), 3.597 (3H, s), 3.606 (1H, d, J=11.4 Hz), 3.896 (3H, s), 3.938 (2H, t, J=6.2 Hz), 4.44-4.51 (2H, m), 6.182 (1H, s), 6.609 (1H, s), 6.69-7.42 (13H, m), 8.50-8.55 (1H, br).

Elemental Analysis (C₄₂H₄₇N₂O₉Cl.0.5H₂O) Cal'd: C, 65.66; H, 6.30; N, 3.65. Found: C, 65.29; H, 6.27; N, 3.62.

### EXAMPLE 125

3-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-(3-phenylpropyloxy)phenyl]propionic Acid

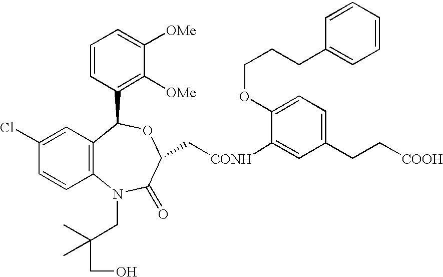

(1) A mixture of 4-hydroxy-3-nitrobenzaldehyde (2 g, 12.0 mmol), potassium carbonate (2.5 g, 18.0 mmol), 1-bromo-3-phenylpropane (2.6 g, 13.2 mmol) and N,N-dimethylformamide (20 ml) was stirred at 60° C. overnight. This mixture was diluted with water and extracted with ethyl acetate (100 ml). The extract was washed with an aqueous saturated sodium chloride solution, dried with anhydrous sodium sulfate and concentrated under reduced pressure. The residue was recrystallized from ethyl acetate-hexane (1:2) to obtain 4-(3-phenylpropyloxy)-3-nitrobenzaldehyde (2.54 g, 8.90 mmol, 74%) as colorless prisms.

m.p. 82.5° C.

IR νₘₐₓ (KBr) cm⁻¹: 1697 (C═O).

¹H-NMR (CDCl₃) δ: 2.200 (2H, quintet, J=7.5 Hz), 2.874 (2H, t, J=7.5 Hz), 4.178 (2H, t, J=7.5 Hz), 7.13-7.33 (5H, m), 8.094 (1H, dd, J=2.2, 8.8 Hz), 8.350 (1H, d, J=2.2 Hz), 9.931 (1H, s).

Elemental Analysis (C₁₆H₁₅NO₄) Cal'd: C, 67.36; H, 5.30; N, 4.91. Found: C, 67.30; H, 5.10; N, 4.72.

(2) A solution of triethylphosphonoacetic acid (3.1 g, 8.83 mmol) in tetrahydrofuran (25 ml) was added to a mixture of 4-(3-phenylpropyloxy)-3-nitrobenzaldehyde (2.4 g, 8.41 mmol) obtained in Example 125-(1), sodium hydride (0.21 g, 8.83 mmol) and tetrahydrofuran (25 ml) at 0° C. The mixture was stirred at room temperature for 1 hour, and the reaction was quenched with a 5% sodium bicarbonate solution. The mixture was diluted with ethyl acetate (100 ml), washed with an aqueous saturated sodium chloride solution, dried with anhydrous sodium sulfate and concentrated under reduced pressure. The residue was purified by recrystallization from ethyl acetate-hexane (1:2) to obtain ethyl 3-[4-(3-phenylpropyloxy)-3-nitrophenyl]-2-propenoate (2.2 g, 6.25 mmol, 75%) as colorless prisms.

m.p. 67-69° C.

IR νₘₐₓ (KBr) cm⁻¹: 1712 (C═O), 1639 (C═C).

¹H-NMR (CDCl₃) δ: 1.339 (3H, t, J=7.4 Hz), 2.10-2.24 (2H, m), 2.859 (2H, t, J=7.3 Hz), 4.108 (2H, t, J=7.3 Hz), 4.269 (1H, d, J=16.0 Hz), 7.634 (1H, dd, J=2.2, 8.8 Hz), 8.013 (1H, d, J=2.2 Hz).

Elemental Analysis (C₂₀H₂₁NO₅) Cal'd: C, 67.59; H, 5.96; N, 3.94. Found: C, 67.61; H, 5.84; N, 3.73.

(3) 10% Palladium carbon (0.2 g) and a 4N hydrogen chloride-ethyl acetate solution (2 ml) were added to a solution of ethyl 3-[4-(3-phenylpropyloxy)-3-nitrophenyl]-2-propionate (2.1 g, 5.91 mmol) obtained in Example 125-(2) in ethanol (40 ml), which was subjected to normal pressure catalytic reduction at room temperature for 4 hours. The catalyst was filtered to remove, and the filtrate was concentrated under reduced pressure. The residue was washed with diethyl ether-hexane (1:1) to obtain ethyl 3-[3-amino-4-(3-phenylpropyloxy)phenyl]-2-propionate hydrochloride (2.1 g, 5.77 mmol, 98%) as a brown oil.

IR νₘₐₓ (KBr) cm⁻¹: 3200-2400 (br, NH₃⁺), 1732 (C═O).

¹H-NMR (CDCl₃) δ: 1.218 (3H, t, J=7.4 Hz), 2.05-2.17 (2H, m), 2.469 (2H, t, J=7.7 Hz), 2.73-2.81 (4H, m), 3.918 (2H, t, J=6.1 Hz), 4.099 (2H, q, J=7.4 Hz), 6.724 (1H, d, J=8.4 Hz), 7.04-7.13 (6H, m), 7.473 (1H, s).

(4) Thionyl chloride (1.1 g, 9.03 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.6 g, 3.01 mmol) obtained in Example 1-(1), N,N-dimethylformamide (0.03 ml) and tetrahydrofuran (15 ml) at room temperature, and the mixture was stirred for 1 hour. The residue obtained by concentration under reduced pressure was dissolved in tetrahydrofuran (10 ml). This solution was added to a mixture of ethyl 3-[3-amino-4-(3-phenylpropyloxe)phenyl]-2-propionate hydrochloride (2.2 g, 6.02 mmol) obtained in Example 125-(3), triethylamine (0.76 g, 7.53 mmol) and tetrahydrofuran (15 ml). The mixture was stirred at room temperature for 30 minutes, and diluted with ethyl acetate (100 ml). This was washed with 1 N hydrochloric acid, an aqueous saturated sodium bicarbonate solution and an aqueous saturated sodium chloride solution, dried with anhydrous sodium sulfate, and concentrated under reduced pressure. The residue was purified by silica gel chromatography [eluent:hexane-ethyl acetate (3:2)] to obtain ethyl 3-[3-[[[(3R,5S)-1-(3-acetoxy-2,2-dimethylpropyl)-7-chloro-5-(2,3-dimethoxyphenyl)-2-oxo-1,2,3,5-tetrhydro-4,1-benzoxazepin-3-yl]acetyl]amino]-4-(3-phenylpropyloxy)phenyl]propionate (1.5 g, 1.81 mmol, 60%) as a brown oil.

IR νₘₐₓ (KBr) cm⁻¹: 3414, 3346 (NH), 1732, 1682 (C═O).

¹H-NMR (CDCl₃) δ: 0.918 (3H, s), 0.984 (3H, s), 1.229 (3H, t, J=7.0 Hz), 2.002 (3H, s), 2.05-2.15 (2H, m), 2.575 (2H, t, J=7.5 Hz), 2.74-2.91 (5H, m), 3.070 (1H, dd, J=7.0, 13.8 Hz), 3.529 (1H, d, J=14.2 Hz), 3.585 (3H, s), 3.693 (1H, d, J=11.0 Hz), 3.820 (1H, d, J=11.0 Hz), 3.878 (3H, s), 3.960 (2H, t, J=6.8 Hz), 4.114 (2H, q, J=7.0 Hz), 4.449 (1H, t, J=7.0 Hz), 4.549 (1H, d, J=14.2 Hz), 6.283 (1H, s), 6.622 (1H, s), 6.70-7.36 (12H, m), 8.162 (1H, brs), 8.211 (1H, d, J=1.8 Hz).

(5) A mixture of ethyl 3-[3-[[[(3R,5S)-1-3-acetoxy-2,2-dimethylpropyl)-7-chloro-5-(2,3-dimethoxyphenyl)-2-oxo-1,2,3,5-tetrahydro-4,1-benzoxazepin-3-yl] acetyl]amino]-4-(3-phenylpropyloxy)phenyl]propionate (1.4 g, 1.69 mmol) obtained in Example 125-(4), a 1N aqueous sodium hydroxide solution (3.7 mmol) and ethanol (15 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate and concentrated under reduced pressure. The residue was purified by recrystallization from ethyl acetate-hexane (1:1) to obtain 3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl] acetyl]amino]-4-(3-phenylpropyloxy) phenyl] propionic acid (1.0 g, 1.32 mmol, 78%) as a colorless powder.

m.p. 162-165° C.

[α]_{D}²²−153.2° (c=0.30, MeOH).

IR νₘₐₓ (KBr) cm⁻¹: 3600-2400 (br, COOH, NH, OH), 1709, 1658 (C═O). ¹H-NMR (CDCl₃) δ: 0.637 (3H, s), 1.029 (3H, s), 2.02-2.15 (2H, m), 2.620 (2H, t, J=7.7 Hz), 2.72-2.90 (5H, m), 3.079 (1H, dd, J=7.0, 14.4 Hz), 3.150 (1H, d, J=11.8 Hz), 3.380 (1H, d, J=14.2 Hz), 3.580 (3H, s), 3.626 (1H, d, J=11.8 Hz), 3.879 (3H, s), 3.92-3.99 (2H, m), 4.44-4.51 (2H, m), 6.181 (1H, s), 6.604 (1H, d, J=1.6 Hz), 6.70-7.36 (12H, m), 8.100 (1H, s), 8.184 (1H, d, J=1.8 Hz).

Elemental Analysis (C₄₂H₄₇N₂O₉Cl.0.5H₂O) Cal'd: C, 65.66; H, 6.30; N, 3.65. Found: C, 65.84; H, 6.11; N, 3.60.

### EXAMPLE 126

3-[3-[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-5,6,7,8-tetrahydronaphthalen-1-yl]propionic Acid

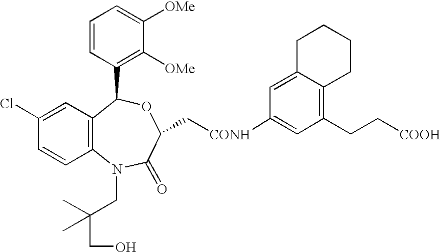

(1) Carbonyldiimidazole (0.86 g, 5.32 mmol) was added to a solution of 3-nitro-5,6,7,8-tetrahydro-1-naphthoic acid (1 g, 4.52 mmol) in tetrahydrofuran (10 ml) at room temperature. The mixture was stirred at room temperature for 6 hours, a magnesium salt of malonic acid monoethyl ester (0.76 g, 2.66 mmol) was added. This mixture was stirred at 60° C. for 1 hour, the reaction solution was diluted with ethyl acetate (100 ml), washed with 1 N hydrochloric acid, an aqueous saturated sodium bicarbonate solution and an aqueous saturated sodium chloride solution, dried with anhydrous sodium sulfate and concentrated under reduced pressure. The residue was purified by silica gel column chromatography [eluent: hexane-ethyl acetate (1:1)] to obtain ethyl 3-(3-nitro-5,6,7,8-tetrahydronaphthalen-1-yl)-3-oxopropionate (0.38 g, 1.30 mmol, 29%) as a colorless oil.

IR νₘₐₓ (KBr) cm⁻¹: 1741, 1697 (C═O).

¹H-NMR (CDCl₃) δ: 1.258 (⅗×3H, t, J=7.2 Hz), 1.346 (⅖×3H, t, J=7.2 Hz), 1.79-1.86 (4H, m), 2.85-3.07 (4H, m), 3.965 (⅗×2H, s), 4.201 (⅗×2H, q, J=7.2 Hz), 4.287 (⅖×2H, q, J=7.2 Hz), 5.298 (⅖×1H, s), 8.03-8.24 (2H, m).

(2) Sodium borohydride (98 mg, 2.59 mmol) was added to a solution of ethyl 3-(3-nitro-5,6,7,8-tetrahydronaphthalen-1-yl)-3-oxopropionate (0.38 g, 1.30 mmol) obtained in Example 126-(1) in methanol (6 ml) at −20° C. The mixture was stirred at −20° C. for 30 minutes, and 1 N hydrochloric acid (3 ml) was added. The mixture was diluted with ethyl acetate (300 ml), washed with water, an aqueous saturated sodium bicarbonate solution and an aqueous saturated sodium chloride solution, dried with anhydrous sodium sulfate, and the residue was purified by silica gel column chromatography [eluent: hexane-ethyl acetate (3:1)] to obtain ethyl 3-(3-nitro-5,6,7,8-tetrahydronaphthalen-1-yl)-3-hydroxypropionate (0.27 g, 0.921 mmol, 71%) as a colorless oil.

IR νₘₐₓ (KBr) cm⁻¹: 3600-3200 (br, OH), 1732 (C═O).

¹H-NMR (CDCl₃) δ: 1.302 (3H, t, J=7.2 Hz), 1.75-1.95 (4H, m), 2.58-2.75 (3H, m), 2.85-2.96 (3H, m), 3.484 (1H, d, J=3.0 Hz), 4.234 (2H, q, J=7.2 Hz), 5.34-5.40 (1H, m), 7.883 (1H, d, J=2.2 Hz), 8.239 (1H, d, J=2.2 Hz).

(3) A mixture of ethyl 3-(3-nitro-5,6,7,8-tetrahydronaphthalen-1-yl)-3-hydroxypropionate (0.27 g, 0.921 mmol) obtained in Example 126-(2), triethylamine (0.11 g, 1.11 mmol), methanesulfonyl chloride (0.12 g, 1.01 mmol) and ethyl acetate (5 ml) was stirred at 0° C. for 30 minutes. 1,8-diazabicyclo[5.4.0]-7-undecene (0.17 g, 1.11 mmol) was added, and the mixture was stirred at 0° C. for 30 minutes. This mixture was diluted with ethyl acetate (50 ml), and washed with 1 N hydrochloric acid (3 ml), an aqueous saturated sodium bicarbonate solution and an aqueous saturated sodium chloride solution. The mixture was dried with anhydrous sodium sulfate, and concentrated under reduced pressure. The residue was purified by silica gel column chromatography [eluent: hexane-ethyl acetate (10:1)] to obtain ethyl 3-(3-nitro-5,6,7,8-tetrahydronaphthalen-1-yl)-2-propenoate (0.26 g, 0.944 mmol, quant) as a colorless powder.

m.p. 95-96° C.

IR νₘₐₓ (KBr) cm⁻¹: 1714 (C═O), 1635 (C═C).

¹H-NMR (CDCl₃) δ: 1.353 (3H, t, J=7.0 Hz), 1.75-1.95 (4H, m), 2.890 (4H, t, J=6.2 Hz), 4.289 (2H, q, J=7.0 Hz), 6.445 (1H, d, J=15.8 Hz), 7.932 (1H, d, J=15.8 Hz), 7.953 (1H, t, J=2.2 Hz), 8.182 (1H, d, J=2.2 Hz).

(4) 10% palladium carbon (0.1 g) was added to a solution of ethyl 3-(3-nitro-5,6,7,8-tetrahydronaphthalen-1-yl)-2-propenoate (0.26 g, 0.944 mmol) obtained in Example 126-(3) in ethyl acetate (10 ml). This suspension was subjected to normal pressure catalytic reduction at room temperature for 2 hours. The catalyst was filtered to remove, and the filtrate was concentrated under reduced pressure to obtain ethyl 3-(3-amino-5,6,7,8-tetrahydronaphthalen-1-yl)propionate (0.19 g, 0.768 mmol, 81%) as a colorless oil.

IR νₘₐₓ (KBr) cm⁻¹: 3435, 3366 (br, NH₂), 1732 (C═O).

¹H-NMR (CDCl₃) δ: 1.260 (3H, t, J=7.4 Hz), 1.66-1.85 (4H, m), 2.49-2.86 (8H, m), 3.4-3.5 (2H, br), 4.146 (2H, q, J=7.4 Hz), 6.323 (1H, d, J=2.2 Hz), 6.382 (1H, d, J=2.2 Hz).

(5) Thionyl chloride (0.25 g, 2.09 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (0.36 g, 0.698 mmol) obtained in Example 1-(1) and N,N-dimethylformamide (0.01 ml) in tetrahydrofuran (5 ml) at room temperature. The mixture was stirred for 1 hour and concentrated under reduced pressure. The residue was dissolved in tetrahydrofuran (5 ml), and the solution was added to a mixture of ethyl 3-(3-amino-5,6,7,8-tetrahydronaphthalen-1-yl)propionate (0.19 g, 0.768 mmol) obtained in Example 126-(4), 4-(dimethylamino)pyridine (0.10 g, 0.838 mmol) and tetrahydrofuran (5 ml). This was stirred at room temperature for 30 minutes, and diluted with ethyl acetate (50 ml). This was washed with 1N hydrochloric acid, an aqueous saturated sodium bicarbonate solution and an aqueous saturated sodium chloride solution, dried with anhydrous sodium sulfate and concentrated under reduced pressure. The residue was purified by silica gel column chromatography [eluent: hexane-ethyl acetate (3:2)] to obtain ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-5,6,7,8-tetrahydronaphthalen-1-yl]propionate (0.23 g, 0.307 mmol, 44%) as a colorless amorphous powder.

[α]_{D}²²−123.8° (c=0.21, MeOH).

IR νₘₐₓ (KBr) cm⁻¹: 3331 (NH), 1738, 1682 (C═O).

¹H-NMR (CDCl₃) δ: 0.956 (3H, s), 1.024 (3H, s), 1.258 (3H, t, J=7.2 Hz), 1.72-1.84 (4H, m), 2.028 (3H, s), 2.50-3.03 (10H, m), 3.532 (1H, d, J=13.8 Hz), 3.617 (3H, s), 3.729 (1H, d, J=11.4 Hz), 3.868 (1H, d, J=11.4 Hz), 3.894 (3H, s), 6.292 (1H, s), 6.637 (1H, d, J=2.2 Hz), 6.96-7.37 (7H, m), 7.671 (1H, brs).

Elemental Analysis (C₄₁H₄₉N₂O₉Cl) Cal'd: C, 65.72; H, 6.59; N, 3.74. Found; C, 65.39; H, 6.65; N, 3.64.

(6) A mixture of ethyl 3-[3-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino-5,6,7,8-tetrahydronaphthalen-1-yl]propionate (0.15 g, 0.200 mmol) obtained in Example 126-(5), a 1 N aqueous sodium hydroxide solution (1 ml) and ethanol (3 ml) was stirred at 60° C. for 30 minutes. This was diluted with water (50 ml), acidified, and extracted with ethyl acetate (50 ml) twice. This was washed with an aqueous saturated sodium chloride solution, dried with anhydrous sodium sulfate, and concentrated under reduced pressure. The residue was purified by recrystallization from ethyl acetate-hexane (1:2) to obtain 3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino-5,6,7,8-tetrahydronaphthalen-1-yl]propionic acid (0.11 g, 0.160 mmol, 80%) as colorless needles.

m.p. 160-162° C.

[α]_{D}²²−124.3° (c=0.14, MeOH).

IR νₘₐₓ (KBr) cm⁻¹: 3600-2400 (br, COOH, OH), 1714, 1657 (C═O). ¹H-NMR (CDCl₃) δ: 0.654 (3H, s), 1.048 (3H, s), 0.70-1.95 (4H, m), 2.56-3.04 (10H, m), 3.188 (1H, d, J=12.0 Hz), 3.384 (1H, d, J=14.4 Hz), 3.610 (3H, s), 3.626 (1H, d, J=12.0 Hz), 3.892 (3H, s), 4.39-4.51 (2H, m), 6.174 (1H, s), 6.622 (1H, d, J=2.0 Hz), 6.97-7.40 (7H, m), 7.823 (1H, brs).

Elemental Analysis (C₃₇H₄₇N₂O₈Cl.H₂O) Cal'd: C, 63.74; H, 6.51; N, 4.02. Found: C, 63.78; H, 6.47; N, 3.92.

### EXAMPLE 127

6-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-naphthoic acid

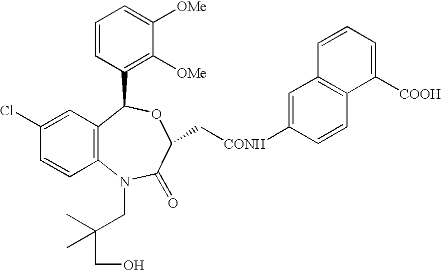

(1) 10% Palladium carbon (0.1 g) was added to a solution of ethyl 6-nitro-1-naphthoate (1.0 g, 4.08 mmol) in ethyl acetate (20 ml), and the mixture was subjected to normal pressure catalytic reduction at room temperature for 3 hours. The catalyst was filtered to remove, and the filtrate was concentrated under reduced pressure. The residue was dissolved in ethyl acetate (50 ml), a 4N hydrogen chloride-ethyl acetate solution (1.5 ml), and concentrated under reduced pressure. The residue was washed with diethyl ether-hexane (1:1) to obtain ethyl 6-amino-1-naphthoate hydrochloride (0.82 g, 3.26 mmol, 80%) as a colorless powder.

m.p. 244-245° C. (dec).

IR νₘₐₓ (KBr) cm⁻: 3300-2400 (br, NH₃⁺), 1712 (C═O).

¹H-NMR (CD₃OD) δ: 1.451 (3H, t, J=7.2 Hz), 4.474 (2H, q, J=7.2 Hz), 7.599 (1H, dd, J=2.2, 9.2 Hz), 7.691 (1H, m), 8.006 (1H, d, J=2.2 Hz), 8.183 (1H, d, J=8.0 Hz), 8.293 (1H, d, J=7.2 Hz), 9.089 (1H, d, J=9.2 Hz).

Elemental Analysis (C₁₃H₁₃NO₂.HCl) Cal'd: C, 62.03; H, 5.61; N, 5.56. Found: C, 61.91; H, 5.63; N, 5.75.

(2) Thionyl chloride (0.7 g, 5.88 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1), N,N-dimethylformamide (0.02 ml) and tetrahydrofuran (10 ml) at room temperature, and the mixture was stirred for 1 hour. The residue obtained by concentration under reduced pressure was dissolved in tetrahydrofuran (10 ml). This solution was added to a mixture of ethyl 6-amino-1-naphthoate hydrochloride (0.53 g, 2.11 mmol) obtained in Example 127-(1), triethylamine (0.48 g, 4.80 mmol) and tetrahydrofuran (10 ml). The mixture was stirred at room temperature for 1 hour, and diluted with ethyl acetate (100 ml). This was washed with 1N hydrochloric acid, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (3:2)] to obtain ethyl 6-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-naphthoate (0.17 g, 0.237 mmol, 11%) as a colorless amorphous powder.

IR νₘₐₓ (KBr) cm⁻¹: 3331 (NH), 1714, 1682 (C═O).

¹H-NMR (CDCl₃) δ: 0.967 (3H, s), 1.029 (3H, s), 1.460 (3H, t, J=7.2 Hz), 2.026 (3H, s), 2.900 (1H, dd, J=5.8, 13.8 Hz), 3.061 (1H, dd, J=7.0, 13.8 Hz), 3.547 (1H, d, J=14.2 Hz), 3.626 (3H, s), 3.738 (1H, d, J=10.8 Hz), 3.882 (1H, d, J=10.8 Hz), 3.896 (3H, s), 4.355 (1H, dd, J=5.8, 7.0 Hz), 4.468 (2H, q, J=7.2 Hz), 4.584 (1H, d, J=14.2 Hz), 6.326 (1H, s), 6.655 (1H, d, J=1.8 Hz), 6.96-7.50 (7H, m), 7.951 (1H, d, J=8.2 Hz), 8.09-8.12 (2H, m), 8.365 (1H, d, J=2.2 Hz), 8.882 (1H, d, J=9.2 Hz).

(3) A mixture of ethyl 6-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-naphthoate (0.17 g, 0.237 mmol) obtained in Example 127-(2), a 1N aqueous sodium hydroxide solution (0.6 ml) and ethanol (3 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by column chromatography [eluent:ethyl acetate-methanol (10:1)] to obtain 6-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-naphthoic acid (50 mg, 0.0790 mmol, 33%) as a colorless amorphous powder.

[α]_{D}²²−98.7° (c=0.14, MeOH).

IR νₘₐₓ (KBr) cm⁻¹: 3600-2400 (br, COOH, OH, NH), 1658 (C═O).

¹H-NMR (CDCl₃) δ: 0.672 (3H, s), 1.058 (3H, s), 2.925 (1H, dd, J=5.8, 14.0 Hz), 3.092 (1H, dd, J=7.4, 14.0 Hz), 3.200 (1H, d, J=11.8 Hz), 3.403 (1H, d, J=14.6 Hz), 3.619 (3H, s), 3.641 (1H, d, J=11.8 Hz), 3.886 (3H, s), 4.47-4.55 (2H, m), 6.229 (1H, s), 6.641 (1H, s), 6.96-7.53 (7H, m), 8.004 (1H, d, J=8.2 Hz), 8.04-8.12 (1H, m), 8.266 (1H, d, J=7.4 Hz), 8.388 (1H, s), 9.001 (1H, d, J=9.2 Hz).

### EXAMPLE 128

3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-naphthoic acid

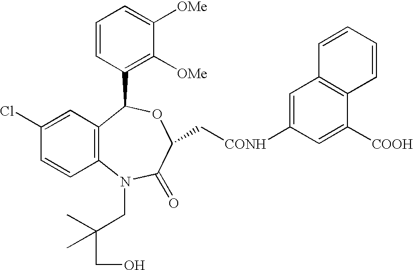

(1) 10% Palladium carbon (0.1 g) was added to a solution of ethyl 3-nitro-1-naphthoate (1.0 g, 4.08 mmol) in ethyl acetate (20 ml), and the mixture was subjected to normal pressure catalytic reduction at room temperature for 3 hours. The catalyst was filtered to remove, and the filtrate was concentrated under reduced pressure. The residue was dissolved in ethyl acetate (50 ml), a 4N hydrogen chloride-ethyl acetate solution (1.5 ml), and concentrated under reduced pressure. The residue was washed with diethyl ether-hexane (1:1) to obtain ethyl 3-amino-1-naphthoate hydrochloride (0.85 g, 3.38 mmol, 83%) as a colorless powder.

m.p. 185-190° C.

IR νₘₐₓ (KBr) cm⁻¹: 3600-2400 (br, NH₃⁺), 1716, 1705 (C═O).

¹H-NMR (CD₃OD) δ: 1.467 (3H, t, J=7.0 Hz), 4.508 (2H, q, J=7.0 Hz), 7.65-7.77 (2H, m), 8.02-8.15 (3H, m), 8.89-8.92 (1H, m).

Elemental Analysis (C₁₃H₁₃NO₂.HCl) Cal'd: C, 62.03; H, 5.61; N, 5.56. Found: C, 62.19; H, 5.70; N, 5.61.

(2) Thionyl chloride (0.7 g, 5.88 mmol) was added to a mixture of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1.0 g, 1.92 mmol) obtained in Example 1-(1), N,N-dimethylformamide (0.02 ml) and tetrahydrofuran (10 ml) at room temperature, and the mixture was stirred for 1 hour. The residue obtained by concentration under reduced pressure was dissolved in tetrahydrofuran (10 ml). This solution was added to a mixture of ethyl 3-amino-1-naphthoate hydrochloride (0.53 g, 2.11 mmol) obtained in Example 128-(1), triethylamine (0.48 g, 4.80 mmol) and tetrahydrofuran (10 ml). The mixture was stirred at room temperature for 30 minutes, and diluted with ethyl acetate (100 ml). This was washed with 1N hydrochloric acid, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (3:2)] to obtain ethyl 3-[[[(3R,5S)-1-(3-acetoxy-2,2-dimethylpropyl)-7-chloro-5-(2,3-dimethylpropyl)-2-oxo-1,2,3,5-tetrahydro-4,1-benzoxazepin-3-yl]acetyl]amino]-1-naphthoate (0.77 g, 1.07 mmol, 56%) as a colorless amorphous powder.

[α]_{D}²²−91.9° (c=0.16, MeOH).

IR νₘₐₓ (KBr) cm⁻¹: 3327 (NH), 1714, 1682 (C═O).

¹H-NMR (CDCl₃) δ: 0.967 (3H, s), 1.029 (3H, s), 1.452 (3H, t, J=7.2 Hz), 2.022 (3H, s), 2.908 (1H, dd, J=5.8, 14.4 Hz), 3.060 (1H, dd, J=7.2, 14.4 Hz), 3.549 (1H, d, J=14.2 Hz), 3.624 (3H, s), 3.738 (1H, d, J=10.8 Hz), 3.880 (1H, d, J=10.8 Hz), 3.890 (3H, s), 4.352 (1H, dd, J=5.8, 7.2 Hz), 4.470 (2H q, J=7.2 Hz), 4.584 (1H, d, J=14.2 Hz), 6.328 (1H, s), 6.655 (1H, d, J=1.8 Hz), 6.96-7.55 (7H, m), 7.79-7.83 (1H, m), 8.085 (1H, d, J=2.2 Hz), 8.10-8.15 (1H, br), 8.451 (1H, d, J=2.2 Hz), 8.78-8.83 (1H, m).

Elemental Analysis (C₃₉H₄₁N₂O₉Cl.0.5H₂O) Cal'd: C, 64.50; H, 5.83; N, 3.86. Found: C, 64.67; H, 5.87; N, 3.63.

(3) A mixture of ethyl 3-[[[(3R,5S)-1-(3-acetoxy-2,2-dimethylpropyl)-7-chloro-5-(2,3-dimethylpropyl)-2-oxo-1,2,3,5-tetrahydro-4,1-benzoxazepin-3-yl]acetyl]amino]-1-naphthoate (0.67 g, 0.934 mmol) obtained in Example 128-(2), a 1N aqueous sodium hydroxide solution (2 ml) and ethanol (10 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by column chromatography [eluent:ethyl acetate-methanol (10:1)] to obtain 3-[[[(3R,5S)-7-chloro-5-(2,3-dimethylpropy)-1-(3-hydroxy-2,2-dimethylpropyl)-2-oxo-1,2,3,5-tetrahydro-4,1-benzoxazepin-3-yl]acetyl]amino]-1-naphthoic acid (50 mg, 0.774 mmol, 83%) as a colorless amorphous powder.

[α]_{D}²²−77.2° (c=0.33, MeOH).

IR νₘₐₓ (KBr) cm⁻¹: 3600-2400 (br, COOH, OH, NH), 1657 (C═O).

¹H-NMR (CD₃OD) δ: 0.882 (3H, s), 0.970 (3H, s), 2.992 (2H, d, J=7.0 Hz), 3.217 (1H, d, J=11.8 Hz), 3.443 (1H, d, J=11.8 Hz), 3.590 (3H, s), 3.691 (1H, d, J=11.0 Hz), 3.876 (3H, s), 4.43-4.58 (2H, m), 6.240 (1H, s), 6.552 (1H, d, J=2.2 Hz), 7.07-7.20 (4H, m), 7.48-7.66 (4H, m), 7.81-7.86 (1H, m), 8.284 (1H, d, J=2.2 Hz), 8.372 (1H, s), 8.80-8.86 (1H, m).

### EXAMPLE 129

5-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-naphthoic acid

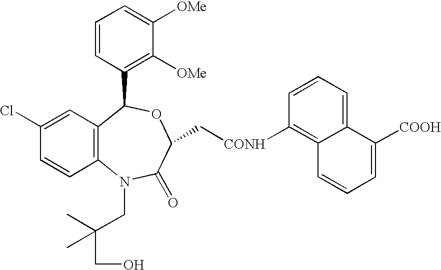

(1) 10% Palladium carbon (0.1 g) was added to a solution of ethyl 5-nitro-1-naphthoate (1.0 g, 4.08 mmol) in ethyl acetate (20 ml), and the mixture was subjected to normal pressure catalytic reduction at room temperature overnight. The catalyst was filtered to remove, and the filtrate was concentrated under reduced pressure. The residue was dissolved in ethyl acetate (50 ml), a 4N hydrogen chloride-ethyl acetate solution (1.5 ml), and concentrated under reduced pressure. The residue was washed with diethyl ether-hexane (1:1) to obtain ethyl 5-amino-1-naphthoate hydrochloride (0.9 g, 3.58 mmol, 88%) as a colorless powder.

m.p. 220-231° C. (dec).

IR νₘₐₓ (KBr) cm⁻¹: 3300-2400 (br, NH₃⁺), 1709 (C═O).

¹H-NMR (CD₃OD) δ: 1.456 (3H, t, J=7.0 Hz), 4.487 (2H, q, J=7.0 Hz), 7.66-7.85 (3H, m), 8.21-8.33 (2H, m), 8.93-9.02 (1H, m).

Elemental Analysis (C₁₃H₁₃NO₂.HCl) Cal'd: C, 62.03; H, 5.61; N, 5.56. Found: C, 61.90; H, 5.59; N, 5.62.

(2) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise under a nitrogen stream, and the mixture was stirred for 30 minutes under ice-cooling. Ethyl 5-amino-1-naphthoate hydrochloride (0.53 g, 2.11 mmol) obtained in Example 129-(1) was added, and pyridine (0.24 g, 3.07 mmol) was added dropwise. A temperature was raised to room temperature, the mixture was stirred for 1 hour, water (50 ml) and 1N hydrochloric acid (3.5 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (1:1)] to obtain ethyl 5-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-naphthoate (1.03 g, 1.44 mmol, 75%) as a colorless amorphous powder.

[α]_{D}²²−156.3° (c=0.17, MeOH).

IR νₘₐₓ (KBr) cm⁻¹: 3258 (NH), 1714, 1678 (C═O).

¹H-NMR (CDCl₃) δ: 0.974 (3H, s), 1.040 (3H, s), 1.460 (3H, t, J=7.2 Hz), 2.031 (3H, s), 2.976 (1H, dd, J=5.2, 14.0 Hz), 3.187 (1H, dd, J=7.8, 14.0 Hz), 3.554 (1H, d, J=14.4 Hz), 3.615 (3H, s), 3.733 (1H, d, J=11.0 Hz), 3.895 (3H, s), 3.899 (1H, d, J=11.0 Hz), 4.42-4.53 (3H, m), 4.602 (1H, d, J=14.4 Hz), 6.346 (1H, s), 6.671 (1H, d, J=2.0 Hz), 6.96-7.62 (7H, m), 7.927 (1H, d, J=7.4 Hz), 8.143 (2H, d, J=7.4 Hz), 8.403 (1H, s), 8.716 (1H, d, J=8.8 Hz).

Elemental Analysis (C₃₉H₄₁N₂O₉Cl) Cal'd: C, 65.31; H, 5.76; N, 3.91. Found: C, 65.04; H, 5.81; N, 3.68.

(3) A mixture of ethyl 5-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-naphthoate (0.92 g, 1.28 mmol) obtained in Example 129-(2), a 1N aqueous sodium hydroxide solution (5 ml) and ethanol (10 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by column chromatography [eluent:ethyl acetate-methanol (1:1)] to obtain 5-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-naphthoic acid (0.67 g, 1.04 mmol, 81%) as a colorless powder.

m.p. 171-174° C.

[α]_{D}²²−158.5° (c=0.29, MeOH).

IR νₘₐₓ (KBr) cm⁻¹: 3500-2400 (br, COOH, OH, NH), 1684, 1653 (C═O).

¹H-NMR (CD₃OD) δ: 0.884 (3H, s), 0.958 (3H, s), 3.096 (2H, d, J=6.6 Hz), 3.236 (1H, d, J=11.4 Hz), 3.464 (1H, d, J=11.4 Hz), 3.604 (3H, s), 3.700 (1H, d, J=13.8 Hz), 3.894 (3H, s), 4.473 (1H, d, J=13.8 Hz), 4.536 (1H, t, J=6.6 Hz), 6.278 (1H, s), 6.552 (1H, d, J=2.2 Hz), 7.11-7.25 (3H, m), 7.43-7.62 (5H, m), 8.19-8.26 (2H, m), 8.81-8.86 (1H, m).

Elemental Analysis (C₃₅H₃₅N₂O₈Cl.0.5H₂O) Cal'd: C, 64.07; H, 5.53; N, 4.27. Found: C, 63.98; H, 5.52; N, 4.01.

### EXAMPLE 130

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-methylbenzoic acid

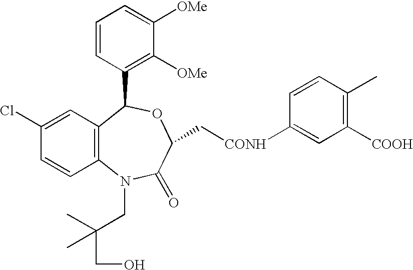

(1) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. Methyl 5-amino-2-methylbenzoate hydrochloride (0.36 g, 2.11 mmol) was added, and pyridine (0.24 g, 3.07 mmol) was added dropwise. A temperature was raised to room temperature, the mixture was stirred for 1 hour, water (50 ml) and 1N hydrochloric acid (3.5 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (1:1)] to obtain methyl 5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-methylbenzoate (0.95 g, 1.42 mmol, 74%) as a colorless amorphous powder.

[α]_{D}²²−115.8° (c=0.15, MeOH).

IR νₘₐₓ (KBr) cm⁻¹: 3319 (NH), 1728, 1682 (C═O).

¹H-NMR (CDCl₃) δ: 0.956 (3H, s), 1.022 (3H, s), 2.022 (3H, s), 2.546 (3H, s), 2.830 (1H, dd, J=6.0, 14.0 Hz), 2.992 (1H, dd, J=7.4, 14.0 Hz), 3.870 (1H, d, J=11.0 Hz), 3.619 (3H, s), 3.731 (1H, d, J=11.0 Hz), 3.870 (1H, d, J=11.0 Hz), 3.879 (3H, s), 3.894 (3H, s), 4.418 (1H, dd, J=6.0, 7.4 Hz), 4.562 (1H, d, J=13.8 Hz), 6.302 (1H, s), 6.644 (1H, d, J=1.8 Hz), 6.96-7.38 (6H, m), 7.619 (1H, dd, J=2.4, 8.4 Hz), 7.86-7.94 (1H, br), 7.970 (1H, d, J=2.4 Hz).

Elemental Analysis (C₃₅H₃₉N₂O₉Cl) Cal'd: C, 63.01; H, 5.89; N, 4.20. Found: C, 63.09; H, 6.01; N, 4.05.

(2) A mixture of methyl 5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-methylbenzoate (0.8 g, 1.20 mmol) obtained in Example 130-(1), a 1N aqueous sodium hydroxide solution (3 ml) and ethanol (8 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by column chromatography [eluent:ethyl acetate-methanol (10:1)] to obtain 5-[[[(3R,5S)-7-chloro-5-(2,3-dimethylpropyl)-1-(3-hydroxy-2,2-dimethylpropyl)-2-oxo-1,2,3,5-tetrahydro-4,1-benzoxazepin-3-yl]acetyl]amino]-2-methylbenzoic acid (0.24 g, 0.393 mmol, 33%) as a colorless amorphous powder.

[α]_{D}²²−136.0° (c=0.29, MeOH).

IR νₘₐₓ (KBr) cm⁻¹: 3600-2400 (br, COOH, OH, NH), 1660 (C═O).

¹H-NMR (CDCl₃) cm⁻¹: 0.654 (3H, s), 1.048 (3H, s), 2.571 (3H, s), 2.862 (1H, dd, J=6.0, 14.4 Hz), 3.046 (1H, dd, J=7.4, 14.4 Hz), 3.196 (1H, d, J=11.6 Hz), 3.388 (1H, d, J=14.0 Hz), 3.606 (3H, s), 3.634 (1H, d, J=11.6 Hz), 3.879 (3H, s), 4.45-4.52 (2H, m), 6.194 (1H, s), 6.617 (1H, s), 6.95-7.34 (6H, m), 7.765 (1H, dd, J=2.2, 8.4 Hz), 8.000 (1H, s), 8.06-8.18 (1H, br).

Elemental Analysis (C₃₂H₃₅N₂O₈Cl.0.5H₂O) Cal'd: C, 61.98; H, 5.85; N, 4.52. Found: C, 62.18; H, 6.20; N, 4.19.

### EXAMPLE 131

5-[[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-fluorobenzoic acid

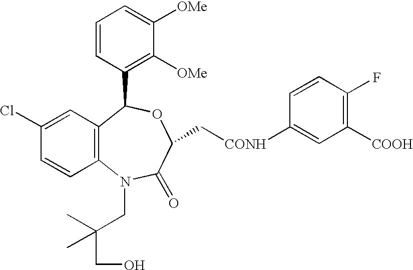

(1) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. 5-amino-2-fluorobenzoate hydrochloride (0.36 g, 2.11 mmol) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (1:1)] to obtain methyl 5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-fluorobenzoate (1.04 g, 1.55 mmol, 81%) as a colorless amorphous powder.

[α]_{D}²²−129.2° (c=0.32, MeOH).

IR νₘₐₓ (KBr) cm⁻¹: 3335 (NH), 1732, 1674 (C═O).

¹H-NMR (CDCl₃) δ: 0.962 (3H, s), 1.018 (3H, s), 2.018 (3H, s), 2.844 (1H, dd, J=5.8, 14.6 Hz), 2.999 (1H, dd, J=7.2, 14.6 Hz), 3.546 (1H, d, J=14.0 Hz), 3.619 (3H, s), 3.736 (1H, d, J=11.4 Hz), 3.875 (1H, d, J=11.4 Hz), 3.894 (3H, s), 3.925 (3H, s), 4.414 (1H, dd, J=5.8, 7.2 Hz), 4.567 (1H, d, J=14.0 Hz), 6.306 (1H, s), 6.653 (1H, d, J=2.0 Hz), 6.96-7.39 (6H, m), 7.75-7.83 (1H, m), 7.9734 (1H, dd, J=2.6, 6.2 Hz), 8.06-8.16 (1H, br).

Elemental Analysis (C₃₄H₃₆N₂O₉ClF) Cal'd: C, 60.85; H, 5.41; N, 4.17. Found: C, 60.68; H, 5.55; N, 3.99.

(2) A mixture of methyl 5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-fluorobenzoate (0.8 g, 1.19 mmol) obtained in Example 131-(1), a 1N aqueous sodium hydroxide solution (3 ml) and ethanol (8 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethanol-hexane (1:3) to obtain 5-[[[(3R,5S)-7-chloro-5-(2,3-dimethylpropyl)-1-(3-hydroxy-2,2-dimethylpropyl)-2-oxo-1,2,3,5-tetrahydro-4,1-benzoxazepin-3-yl]acetyl]amino]-2-fluorobenzoic acid (0.46 g, 0.748 mmol, 63%) as a colorless powder.

[α]_{D}²²−147.1° (c=0.14, MeOH).

IR νₘₐₓ (KBr) cm⁻¹: 3400-2400 (br, COOH, OH, NH), 1685, 1655 (C═O).

¹H-NMR (CDCl₃) δ: 0.685 (3H, s), 1.027 (3H, s), 2.876 (1H, dd, J=6.2, 14.6 Hz), 3.030 (1H, dd, J=7.4, 14.6 Hz), 3.138 (1H, d, J=11.8 Hz), 3.448 (1H, d, J=14.2 Hz), 3.572 (1H, d, J=11.8 Hz), 3.588 (3H, s), 3.896 (3H, s), 4.43-4.54 (2H, m), 6.183 (1H, s), 6.608 (1H, s), 6.96-7.43 (6H, m), 7.84-7.94 (1H, m), 8.045 (1H, dd, J=2.6, 6.2 Hz), 9.694 (1H, s).

Elemental Analysis (C₃₁H₃₂N₂O₈ClF) Cal'd: C, 60.54; H, 5.24; N, 4.55. Found: C, 60.52; H, 5.39; N, 4.32.

### EXAMPLE 132

2-[4-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyloxy]-2,2-difluoroacetic acid

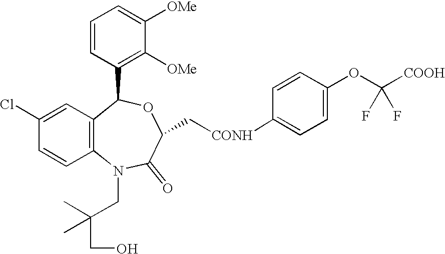

(1) A mixture of 4-nitrophenol (1 g, 7.19 mmol), 1,8-diazabicyclo[5.4.0]-7-undecene (1.3 g, 8.63 mmol), ethyl bromodifluoroacetate (1.75 g, 8.63 mmol) and tetrahydrofuran (10 ml) was stirred at 60° C. for 1 hour. This mixture was diluted with water, and extracted with ethyl acetate (100 ml). The extract was washed with a 1N aqueous sodium hydroxide solution, a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (10:1)] to obtain ethyl 2,2-difluoro-2-(4-nitrophenyloxy)acetate (0.86 g, 3.29 mmol, 46%) as a colorless oil.

IR νₘₐₓ (KBr) cm⁻¹: 1778 (C═O).

¹H-NMR (CDCl₃) δ: 1.397 (3H, t, J=7.0 Hz), 4.426 (2H, q, J=7.0 Hz), 7.380 (2H, d, J=9.2 Hz), 8.279 (2H, d, J=9.2 Hz).

(2) 10% Palladium carbon (0.2 g) and a 4N hydrogen chloride-ethyl acetate solution (1 ml) were added to a solution of ethyl 2,2-difluoro-2-(4-nitrophenyloxy)acetate (0.86 g, 3.29 mmol) obtained in Example 132-(1) in ethanol (20 ml) and the mixture was subjected to normal pressure catalytic reduction at room temperature for 2 hours. The catalyst was filtered to remove, and the filtrate was concentrated under reduced pressure. The residue was washed with hexane to obtain ethyl 2-(4-aminophenyloxy)-2,2-difluoroacetate hydrochloride (0.73 g, 2.73 mmol, 83%) as a colorless powder.

m.p. 193-199° C. (dec).

IR νₘₐₓ (KBr) cm⁻¹: 3200-2400 (br, NH₃⁺), 1774 (C═O).

¹H-NMR (CD₃OD) δ: 1.335 (3H, t, J=7.4 Hz), 4.386 (2H, q, J=7.4 Hz), 7.28-7.51 (4H, m).

Elemental Analysis (C₁₀H₁₁NO₃F₂.HCl) Cal'd: C, 44.87; H, 4.52; N, 5.23. Found: C, 44.49; H, 4.30; N, 5.32.

(3) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. Ethyl 2-(4-aminophenyloxy)-2,2-difluoroacetate hydrochloride (0.56 g, 2.11 mmol) obtained in Example 132-(2) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (3:2)] to obtain ethyl 2-[4-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]phenyloxy]-2,2-difluoroacetate (1.1 g, 1.50 mmol, 78%) as a colorless amorphous powder.

[α]_{D}²²−117.4° (c=0.12, MeOH).

IR νₘₐₓ (KBr) cm⁻¹: 3341 (NH), 1778, 1738, 1682 (C═O).

¹H-NMR (CDCl₃) δ: 0.956 (3H, s), 1.016 (3H, s), 1.375 (3H, t, J=7.4 Hz), 2.018 (3H, s), 2.825 (1H, dd, J=5.4, 13.8 Hz), 2.995 (1H, dd, J=7.6, 13.8 Hz), 3.535 (1H, d, J=14.0 Hz), 3.615 (3H, s), 3.728 (1H, d, J=11.0 Hz), 3.873 (1H, d, J=11.0 Hz), 3.892 (3H, s), 4.33-4.40 (3H, m), 4.555 (1H, d, J=14.0 Hz), 6.297 (1H, s), 6.644 (1H, d, J=1.8 Hz), 6.96-7.52 (9H, m), 7.996 (1H, brs).

Elemental Analysis (C₃₆H₃₉N₂O₁₀ClF₂) Cal'd: C, 58.98; H, 5.36; N, 3.82. Found: C, 59.04; H, 5.48; N, 3.81.

(4) A mixture of ethyl 2-[4-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]phenyloxy]-2,2-difluoroacetate (1 g, 1.36 mmol) obtained in Example 132-(3), a 1N aqueous sodium hydroxide solution (3 ml) and ethanol (10 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:1) to obtain 2-[4-[[[(3R,5S)-7-chloro-5-(2,3-dimethylpropyl)-1-(3-hydroxy-2,2-dimethylpropyl)-2-oxo-1,2,3,5-tetrahydro-4,1-benzoxazepin-3-yl]acetyl]amino]phenyloxy]-2,2-difluoroacetic acid (0.63 g, 0.950 mmol, 70%) as a colorless powder.

m.p. 149-150° C.

[α]_{D}²²−123.6° (c=0.21, MeOH).

IR νₘₐₓ (KBr) cm⁻¹: 3600-2400 (br, COOH, OH, NH), 1768, 1653 (C═O).

¹H-NMR (CDCl₃) δ: 0.650 (3H, s), 1.037 (3H, s), 2.848 (1H, dd, J=5.8, 13.8 Hz), 3.007 (1H, dd, J=7.8, 13.8 Hz), 3.245 (1H, d, J=12.2 Hz), 3.391 (1H, d, J=14.4 Hz), 3.591 (3H, s), 3.626 (1H, d, J=12.2 Hz), 3.885 (3H, s), 4.39-4.46 (2H, m), 6.156 (1H, s), 6.626 (1H, d, J=1.8 Hz), 6.96-7.51 (9H, m), 8.16-8.24 (1H, br).

Elemental Analysis (C₃₂H₃₃N₂O₉ClF₂.0.3AcOEt.H₂O) Cal'd: C, 56.36; H, 5.33; N, 3.96. Found: C, 56.72; H, 5.45; N, 3.97.

### EXAMPLE 133

2-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyloxy]-2,2-difluoroacetic acid

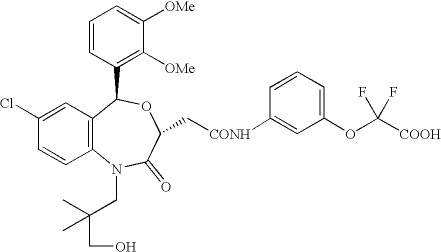

(1) A mixture of 3-nitrophenol (1 g, 7.19 mmol), 1,8-diazabicyclo[5.4.0]-7-undecene (1.3 g, 8.63 mmol), ethyl bromodifluoroacetate (1.75 g, 8.63 mmol) and tetrahydrofuran (10 ml) was stirred at 60° C. for 1 hour. This mixture was diluted with water, and extracted with (100 ml). The extract was washed with a 1N aqueous sodium hydroxide solution, a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (10:1)] to obtain ethyl 2,2-difluoro-2-(3-nitrophenyloxy)acetate (1.2 g, 4.59 mmol, 64%) as a colorless oil.

IR νₘₐₓ (KBr) cm⁻¹: 1778 (C═O).

¹H-NMR (CDCl₃) δ: 1.408 (3H, t, J=7.4 Hz), 4.434 (2H, q, J=7.4 Hz), 7.58-7.60 (2H, m), 8.12-8.19 (2H, m).

(2) 10% palladium carbon (0.2 g) was added to a solution of ethyl 2,2-difluoro-2-(4-nitrophenyloxy)acetate (1.2 g, 4,59 mmol) obtained in Example 133-(1) in ethanol (20 ml) and the mixture was subjected to normal pressure catalytic reduction at room temperature for 2 hours. The catalyst was filtered to remove, a 4N hydrogen chloride-ethyl acetate solution (2 ml) was added, and the filtrate was concentrated under reduced pressure. The residue was washed with ethyl acetate-hexane (1:1) to obtain ethyl 2-(3-aminophenyloxy)-2,2-difluoroacetate hydrochloride (1.1 g, 4.11 mmol, 90%) as a colorless powder.

m.p. 176-179° C. (dec).

IR νₘₐₓ (KBr) cm⁻¹: 3200-2400 (br, NH₃⁺), 1770 (C═O).

¹H-NMR (CD₃OD) δ: 1.335 (3H, t, J=7.0 Hz), 4.391 (2H, q, J=7.0 Hz), 7.31-7.38 (3H, m), 7.57-7.66 (1H, m).

Elemental Analysis (C₁₀H₁₁NO₃F₂.HCl) Cal'd: C, 44.87; H, 4.52; N, 5.23. Found: C, 44.68; H, 4.55; N, 5.43.

(3) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. Ethyl 2-(3-aminophenyloxy)-2,2-difluoroacetate hydrochloride (0.56 g, 2.11 mmol) obtained in Example 133-(2) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent: hexane-ethyl acetate (3:2)] to obtain ethyl 2-[3-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]phenyloxy]-2,2-difluoroacetate (1.0 g, 1.38 mmol, 72%) as a colorless amorphous powder.

[α]_{D}²²=−101.1° (c=0.11, MeOH).

IR νₘₐₓ (KBr) cm⁻¹: 3325 (NH), 1776, 1738, 1680 (C═O).

¹H-NMR (CDCl₃) δ: 0.958 (3H, s), 1.020 (3H, s), 1.368 (3H, t, J=7.4 Hz), 2.018 (3H, s), 2.828 (1H, dd, J=5.6, 14.0 Hz), 3.003 (1H, dd, J=7.4, 14.0 Hz), 3.541 (1H, d, J=14.4 Hz), 3.617 (3H, s), 3.731 (1H, d, J=11.0 Hz), 3.872 (1H, d, J=11.0 Hz), 3.892 (3H, s), 4.33-4.44 (3H, m), 4.563 (1H, d, J=14.4 Hz), 6.299 (1H, s), 6.650 (1H, d, J=2.0 Hz), 6.96-7.51 (9H, m), 8.049 (1H, brs).

Elemental Analysis (C₃₆H₃₉N₂O₁₀ClF₂) Cal'd: C, 58.98; H, 5.36; N, 3.82. Found: C, 58.80; H, 5.46; N, 3.69.

(4) A mixture of ethyl 2-[3-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]phenyloxy]-2,2-difluoroacetate (0.9 g, 1.23 mmol) obtained in Example 133-(3), a 1N aqueous sodium hydroxide solution (3 ml) and ethanol (9 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:1) to obtain 2-[3-[[[(3R,5S)-7-chloro-5-(2,3-dimethylpropyl)-1-(3-hydroxy-2,2-dimethylpropyl)-2-oxo-1,2,3,5-tetrahydro-4,1-benzoxazepin-3-yl]acetyl]amino]phenyloxy]-2,2-difluoroacetic acid (0.27 g, 0.407 mmol, 33%) as a colorless powder.

m.p. 119-121° C.

[α]_{D}²²=−120.9° (c=0.17, MeOH).

IR νₘₐₓ (KBr) cm⁻¹: 3600-2400 (br, COOH, OH, NH), 1770, 1651 (C═O).

¹H-NMR (CDCl₃) δ: 0.657 (3H, s), 1.044 (3H, s), 2.845 (1H, dd, J=4.8, 14.8 Hz), 3.015 (1H, dd, J=7.2, 14.8 Hz), 3.270 (1H, d, J=11.2 Hz), 3.409 (1H, d, J=14.6 Hz), 3.593 (3H, s), 3.624 (1H, d, J=11.2 Hz), 3.886 (3H, s), 4.39-4.46 (2H, m), 6.150 (1H, s), 6.637 (1H, d, J=1.8 Hz), 6.96-7.46 (9H, m), 8.341 (1H, brs).

Elemental Analysis (C₃₂H₃₃N₂O₉ClF₂.AcOEt.H₂O) Cal'd: C, 6.21; H 5.63; N, 3.64. Found: C, 55.96; H, 5.56; N, 3.72.

### EXAMPLE 134

3-[5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-2-fluorophenyl]propionic Acid

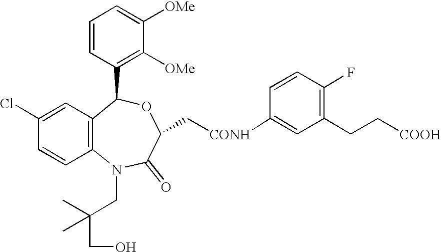

(1) A mixture of 2-chloro-5-nitrocinnamic acid (2 g, 8.79 mmol), potassium carbonate (1.5 g, 10.5 mmol), iodomethane (1.4 g, 9.67 mmol) and N,N-dimethylformamide (20 ml) was stirred at room temperature for 3 hours. This mixture was diluted with water, and extracted with ethyl acetate (100 ml). The extract was washed with an aqueous saturated sodium chloride solution, dried with anhydrous sodium sulfate, and concentrated under reduced pressure. The residue was recrystallized from ethyl acetate-hexane (1:3) to obtain methyl 3-(2-chloro-5-nitrophenyl)-2-propenoate (1.4 g, 5.79 mmol, 66%) as pale yellow needles.

m.p. 165-166° C.

IR νₘₐₓ (KBr) cm⁻¹: 1714 (C—O), 1601 (C═C).

¹H-NMR (CDCl₃) δ: 3.861 (3H, s), 6.586 (1H, d, J=16.0 Hz), 7.619 (1H, d, J=8.8 Hz), 8.057 (1H, d, J=16.0 Hz), 8.171 (1H, dd, J=3.0, 8.8 Hz), 8.489 (1H, d, J=3.0 Hz).

Elemental Analysis (C₁₀H₁₁NO₄Cl) Cal'd: C, 49.71; H, 3.34; N, 5.80. Found: C, 49.66; H, 3.18; N, 5.81.

(2) A mixture of methyl 3-(2-chloro-5-nitrophenyl)-2-propenoate (1.3 g, 5.38 mmol) obtained in Example 134-(1), potassium fluoride (0.75 g, 12.9 mmol) and dimethyl sulfoxide (6 ml) was stirred at 130° C. for 10 hours. This mixture was diluted with ethyl acetate (100 ml), washed with water, a 1 N aqueous sodium hydroxide solution and an aqueous saturated sodium chloride solution, dried with anhydrous sodium sulfate, and concentrated under reduced pressure. The residue was purified by column chromatography [eluent:hexane-ethyl acetate (10:1)] and recrystallization from ethyl acetate-hexane (1:2) to obtain methyl 3-(2-fluoro-5-nitrophenyl)-2-propenoate (0.65 g, 2.89 mmol, 54%) as colorless needles.

m.p. 134-135° C.

IR νₘₐₓ (KBr) cm⁻¹: 1720 (C═O), 1645, 1622 (C═C).

¹H-NMR (CDCl₃) δ: 3.852 (3H, s), 6.668 (1H, d, J=16.6 Hz), 7.289 (1H, t, J=9.2 Hz), 7.808 (1H, d, J=16.6 Hz), 8.264 (1H, ddd, J=2.8, 4.2, 9.2 Hz), 8.476 (1H, dd, J=2.8, 6.2 Hz).

Elemental Analysis (C₁₀H₈NO₄F) Cal'd: C, 53.34; H, 3.58; N, 6.22. Found: C, 53.18; H, 3.43; N, 6.25.

(3) 10% Palladium carbon (0.1 g) was added to a solution of 3-(2-fluoro-5-nitrophenyl)-2-propenoate (0.5 g, 2.22 mmol) obtained in Example 134-(2) in ethyl acetate (10 ml) and the mixture was subjected to normal pressure catalytic reduction at room temperature for 4 hours. The catalyst was filtered to remove, and the filtrate was concentrated under reduced pressure. The residue was dissolved in ethyl acetate (50 ml), a 4N hydrogen chloride-ethyl acetate solution (1 ml) was added, and concentrated under reduced pressure. The residue was washed with ethyl acetate-hexane (1:1) to obtain methyl 3-(5-amino-2-fluorophenyl)propionate hydrochloride (0.5 g, 2.14 mmol, 96%) as a colorless powder.

m.p. 137-138° C. (decomposition).

IR νₘₐₓ (KBr) cm⁻¹: 3300-2400 (br, NH₃⁺), 1728 (C═O).

¹H-NMR (CD₃OD) δ: 2.679 (2H, t, J=7.4 Hz), 3.004 (2H, t, J=7.4 Hz), 3.646 (3H, s), 7.19-7.36 (3H, m).

Elemental Analysis (C₁₀H₁₂NO₂F.HCl) Cal'd: C, 51.40; H, 5.61; N, 5.99. Found: C, 51.30; H, 5.52; N, 6.00.

(4) Triethylamine (0.14 g, 1.39 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (0.7 g, 1.35 mmol) obtained in Example 1-(1) in N,N-dimethylformamide (4 ml) at room temperature. The mixture was ice-cooled, isobutyl chloroformate (0.7 g, 1.35 mmol) was added dropwise over 10 minutes under a nitrogen stream, and the mixture was stirred for 30 minutes under ice-cooling. Methyl 3-(5-amino-2-fluorophenyl)propionate (0.35 g, 1.48 mmol) obtained in Example 134-(3) was added, and pyridine (0.17 g, 2.15 mmol) was added. A temperature was raised to room temperature, the mixture was stirred for 1 hour, water (50 ml) and 1N hydrochloric acid (2.5 ml) were added to the reaction solution, and extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (1:1)] to obtain methyl 3-[5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-fluoropheny]propionate (0.74 g, 1.06 mmol, 78%) as a colorless amorphous powder.

[α]_{D}²²=−141.9° (c=0.14, MeOH).

IR νₘₐₓ (KBr) cm⁻¹: 3327 (NH), 1738, 1678 (C═O).

¹H-NMR (CDCl₃) δ: 0.960 (3H, s), 1.020 (3H, s), 2.020 (3H, s), 2.617 (1H, t, J=7.5 Hz), 2.807 (1H, dd, J=5.8, 14.2 Hz), 2.90-3.04 (3H, m), 3.542 (1H, d, J=14.2 Hz), 3.617 (3H, s), 3.678 (3H, s), 3.732 (1H, d, J=11.2 Hz), 3.873 (1H, d, J=11.2 Hz), 3.896 (3H, s), 4.406 (1H, dd, J=5.8, 7.4 Hz), 4.558 (1H, d, J=14.2 Hz), 6.297 (1H, s), 6.645 (1H, d, J=1.8 Hz), 6.90-7.39 (8H, m), 7.88 (1H, brs).

Elemental Analysis (C₃₆H₄₀N₂O₉ClF) Cal'd: C, 61.84; H, 5.77; N, 4.01. Found: C, 61.93; H, 6.05; N, 3.84.

(5) A mixture of methyl 3-[5-[[[(3R,5S)-1-(3-acetoxy-2,2-dimethylpropyl)-7-chloro-5-(2,3-dimethylphenyl)-2-oxo-1,2,3,5-tetrahydro-4,1-benzoxazepin-3-yl]acetyl]amino]-2-fluorophenyl]propionate (0.64 g, 0.915 mmol) obtained in Example 134-(4), a 1N aqueous sodium hydroxide solution (2 ml) and ethanol (6 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:2) to obtain 3-[5-[[[(3R,5S)-7-chloro-5-(2,3-dimethylpropyl)-1-(3-hydroxy-2,2-dimethylpropyl)-2-oxo-1,2,3,5-tetrahydro-4,1-benzoxazepin-3-yl]acetyl]amino]-2-fluoropheny]propionic acid (0.48 g, 0.746 mmol, 82%) as a colorless powder.

m.p. 123-125° C.

[α]_{D}²²=−134.3° (c=0.24, MeOH).

IR νₘₐₓ (KBr) cm⁻¹: 3600-2400 (br, COOH, OH, NH), 1718, 1655 (C═O).

¹H-NMR (CDCl₃) δ: 0.656 (3H, s), 2.641 (2H, t, J=7.0 Hz), 2.812 (1H, dd, J=5.8, 14.6 Hz), 2.940 (2H, t, J=7.0 Hz), 2.992 (1H, dd, J=7.8, 14.6 Hz), 3.192 (1H, d, J=12.4 Hz), 3.391 (1H, d, J=14.4 Hz), 3.603 (3H, s), 3.614 (1H, d, J=12.4 Hz), 3.890 (3H, s), 4.426 (1H, dd, J=5.8, 7.8 Hz, 4.466 (1H, d, J=14.4 Hz), 6.174 (1H, s), 6.627 (1H, d, J=2.2 Hz), 6.90-7.41 (8H, m), 8.101 (1H, s).

Elemental Analysis (C₃₃H₃₆N₂O₈ClF.AcOEt) Cal'd: C, 60.78; H, 6.06; N, 3.83. Found: C, 60.62; H, 6.13; N, 3.79.

### EXAMPLE 135

5-[3-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]pentanoic acid

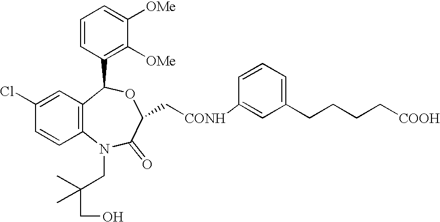

(1) Carbonyldiimidazole (6.8 g, 41.7 mmol) was added to a solution of 3-nitrocinnamic acid (5 g, 25.9 mmol) in tetrahydrofuran (50 ml) at room temperature. The mixture was stirred at room temperature for 1.5 hours, and magnesium chloride (2.5 g, 25.9 mmol) and a potassium salt of malonic acid monoethyl ester (4.4 g, 25.9 mmol) were added. This mixture was stirred at 60° C. for 1 hour, the reaction solution was diluted with ethyl acetate (100 ml), washed with 1N hydrochloric acid, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (3:1)] and recrystallization from ethyl acetate-hexane (1:5) to obtain ethyl 5-(3-nitrophenyl)-3-oxo-4-pentenoate (4.3 g, 16.3 mmol, 63%) as pale yellow prisms.

m.p. 92-93° C.

IR νₘₐₓ (KBr) cm⁻: 1755, 1651 (C═O), 1614, 1606 (C═C).

¹H-NMR (CDCl₃) δ: 1.298 (2/7×3H, t, J=7.0 Hz), 1.333 (5/7×3H, t, J=7.0 Hz), 3.722 (2/7×2H, s), 4.242 (2/7×2H, q, J=7.0 Hz), 4.259 (5/7×2H, q, J=7.0 Hz), 5.238 (5/7×1H, s), 6.558 (5/7×1H, dd, J=1.4, 16.0 Hz), 6.943 (2/7×1H, d, J=16.0 Hz), 7.42-7.89 (3H, m), 8.15-8.42 (2H, m).

Elemental Analysis (C₁₃H₁₃NO₅) Cal'd: C, 59.31; H, 4.98; N, 5.32. Found: C, 59.31; H, 4.96; N, 5.44.

(2) Sodium borohydride (0.72 g, 19.0 mmol) was added to a solution of ethyl 5-(3-nitrophenyl)-3-oxo-4-pentenoate (4.2 g, 15.8 mmol) obtained in Example 135-(1) in methanol (50 ml) at −20° C. The mixture was stirred at −20° C. for 30 minutes, and 1N hydrochloric acid (20 ml) was added. This mixture was diluted with ethyl acetate (150 ml), washed with water, an aqueous saturated sodium bicarbonate solution and an aqueous saturated sodium chloride solution, dried with anhydrous sodium sulfate, and the residue was purified by silica gel column chromatography [eluent:hexane-ethyl acetate (2:1)] to obtain ethyl 3-hydroxy-5-(3-nitrophenyl)-4-penteonate (3.7 g, 13.8 mmol, 88%) as a colorless oil.

IR νₘₐₓ (KBr) cm⁻¹: 3600-3200 (br, OH), 1732 (C═O).

¹H-NMR (CDCl₃) δ: 1.293 (3H, t, J=7.4 Hz), 2.609 (1H, dd, J=8.0, 16.4 Hz), 2.721 (1H, dd, J=4.4, 16.4 Hz), 3.291 (1H, d, J=4.4 Hz), 4.212 (2H, q, J=7.4 Hz), 4.71-4.82 (1H, m), 6.374 (1H, dd, J=5.4, 16.0 Hz), 6.759 (1H, dd, J=1.4, 16.0 Hz), 7.491 (1H, t, J=8.0 Hz), 7.66-7.70 (1H, m), 8.07-8.25 (2H, m).

Elemental Analysis (C₁₃H₁₅NO₅) Cal'd: C, 58.86; H, 5.70; N, 5.28. Found: C, 58.53; H, 5.58; N, 5.26.

(3) A mixture of ethyl 3-hydroxy-5-(3-nitrophenyl)-4-pentenoate (3.4 g, 12.8 mmol) obtained in Example 135-(2), triethylamine (1.6 g, 15.4 mmol), methanesulfonyl chloride (1.6 g, 14.1 mmol) and ethyl acetate (30 ml) was stirred at 0° C. for 30 minutes. 1,8-diazabicyclo[5.4.0]-7-undecene (2.3 g, 15.4 mmol) was added, and this mixture was stirred at 0° C. for 30 minutes. This mixture was diluted with ethyl acetate (50 ml), washed with 1N hydrochloric acid (35 ml), an aqueous saturated sodium bicarbonate solution and an aqueous saturated sodium chloride solution. The mixture was dried with anhydrous sodium sulfate, and concentrated under reduced pressure. The residue was purified by silica gel column chromatography [eluent:hexane-ethyl acetate (10:1)] and recrystallization from ethyl acetate-hexane (1:5) to obtain ethyl 5-(3-nitrophenyl)-2,4-pentadienoate (2.3 g, 9.30 mmol, 73%) as colorless needles.

m.p. 100-101° C.

IR νₘₐₓ (KBr) cm⁻¹: 1705 (C═O), 1631, 1614 (C═C).

¹H-NMR (CDCl₃) δ: 1.332 (3H, t, J=7.4 Hz), 4.251 (2H, q, J=7.4 Hz), 6.090 (1H, d, J=15.4 Hz), 6.916 (1H, d, J=14.6 Hz), 7.035 (1H, d, J=14.6 Hz), 7.448 (1H, ddd, J=1.4, 8.4, 15.4 Hz), 7.540 (1H, t, J=8.2 Hz), 7.74-8.33 (3H, m).

Elemental Analysis (C₁₃H₁₃NO₄) Cal'd: C, 62.44; H, 5.81; N, 4.18. Found: C, 63.13; H, 5.19; N, 5.68.

(4) 10% Palladium carbon (0.2 g) was added to a solution of ethyl 5-(3-nitrophenyl)-2,4-pentadienoate (2.2 g, 8.54 mmol) obtained in Example 135-(3) in ethyl acetate (100 ml). This suspension was subjected to normal pressure catalytic reduction at room temperature overnight. The catalyst was filtered to remove, and the filtrate was concentrated under reduced pressure. The residue was diluted with ethyl acetate (50 ml), and a 4N solution of hydrogen chloride in ethyl acetate (3 ml) was added. The solvent was distilled off, and the residue was washed with hexane to obtain ethyl 5-(3-aminophenyl)pentanoate hydrochloride (2.4 g, 9.31 mmol, quant) as a colorless powder.

m.p. 90-91° C.

IR νₘₐₓ (KBr) cm⁻¹: 3600-2400 (br, NH₂), 1732 (C═O).

¹H-NMR (CD₃OD) δ: 1.227 (3H, t, J=7.2 Hz), 1.60-1.75 (4H, m), 2.345 (2H, t, J=7.0 Hz), 2.707 (2H, t, J=7.0 Hz), 4.100 (2H, quant), J=7.2 Hz), 7.19-7.49 (4H, m).

Elemental Analysis (C₁₃H₁₉NO₂.HCl) Cal'd: C, 60.58; H, 7.82; N, 5.43. Found: C, 60.83; H, 7.89; N, 5.37.

(5) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. Ethyl 5-(3-aminophenyl)pentanoate hydrochloride (0.54 g, 2.11 mmol) obtained in Example 135-(4) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and the mixture was extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by column chromatography [eluent:hexane-ethyl acetate (3:2)] to obtain ethyl 5-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]pentanoate (1.05 g, 1.45 mmol, 76%) as a colorless amorphous powder.

[α]_{D}²²−133.4° (c=0.22, MeOH).

IR νₘₐₓ (KBr) cm⁻¹: 3333 (NH), 1732, 1682 (C═O).

¹H-NMR (CDCl₃) δ: 0.958 (3H, s), 1.024 (3H, s), 1.245 (3H, t, J=7.4 Hz), 1.62-1.67 (4H, m), 2.026 (3H, s), 2.313 (2H, t, J=7.0 Hz), 2.604 (2H, t, J=7.0 Hz), 2.812 (1H, dd, J=5.8, 13.8 Hz), 2.995 (1H, dd, J=7.4, 13.8 Hz), 3.537 (1H, d, J=13.8 Hz), 3.619 (3H, s), 3.730 (1H, d, J=11.4 Hz), 3.873 (1H, d, J=11.4 Hz), 3.894 (3H, s), 4.118 (2H, q, J=7.4 Hz), 4.410 (1H, dd, J=5.8, 7.4 Hz), 4.562 (1H, d, J=13.8 Hz), 6.298 (1H, s), 6.640 (1H, d, J=2.0 Hz), 6.90-7.38 (9H, m), 7.791 (1H, brs).

Elemental Analysis (C₃₉H₄₇N₂O₉Cl) Cal'd: C, 64.77; H, 6.55; N, 3.87. Found: C, 64.57; H, 6.56; N, 3.79.

(6) A mixture of ethyl 5-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]phenyl]pentanoate (0.9 g, 1.21 mmol) obtained in Example 135-(5), ethanol (10 ml), and a 1N aqueous sodium hydroxide solution (3 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:1) to obtain 5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]phenyl]pentanoic acid (0.79 g, 1.21 mmol, quant) as a colorless powder.

m.p. 117-119° C.

[α]_{D}²²−135.6° (c=0.22, MeOH).

IR νₘₐₓ (KBr) cm⁻¹: 3500-2400 (br, COOH, OH, NH), 1712, 1651 (C═O).

¹H-NMR (CDCl₃) δ: 0.654 (3H, s), 1.044 (3H, s), 1.64-1.69 (4H, m), 2.33-2.39 (2H, m), 2.57-2.65 (2H, m), 2.814 (1H, dd, J=5.4, 14.2 Hz), 3.030 (1H, dd, J=7.8, 14.2 Hz), 3.183 (1H, d, J=12.2 Hz), 3.380 (1H, d, J=14.4 Hz), 3.606 (3H, s), 3.629 (1H, d, J=12.2 Hz), 3.890 (3H, s), 4.40-4.51 (2H, m), 6.181 (1H, s), 6.620 (1H, d, J=2.0 Hz), 6.90-7.40 (9H, m), 7.888 (1H, brs).

Elemental Analysis (C₃₅H₄₁N₂O₈Cl.AcOEt) Cal'd: C, 63.19; H, 6.66; N, 3.78. Found: C, 63.10; H, 6.59; N, 3.63.

### EXAMPLE 136

2-[4-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-fluorophenyloxy]acetic acid]

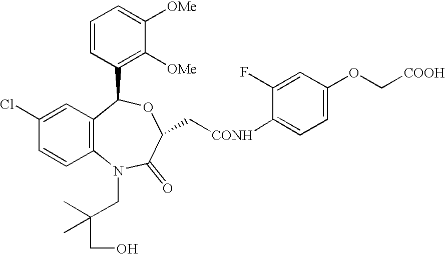

(1) A mixture of 3-fluoro-4-nitrophenol (1.5 g, 8.55 mmol), potassium carbonate (1.5 g, 10.5 mmol), methyl bromoacetate (1.8 g, 11.5 mmol) and N,N-dimethylformamide (15 ml) was stirred at room temperature for 1 hour. This mixture was diluted with water, and extracted with ethyl acetate (100 ml). The extract was washed with an aqueous saturated sodium chloride solution, dried with anhydrous sodium sulfate, and concentrated under reduced pressure. The residue was recrystallized from ethyl acetate-hexane (1:2) to obtain methyl 2-[(3-fluoro-4-nitrophenyl)oxy]acetate (1.6 g, 7.16 mmol, 75%) as colorless needles.

m.p. 93-94° C.

IR νₘₐₓ (KBr) cm⁻¹: 1761 (C═O).

¹H-NMR (CDCl₃) δ: 3.839 (3H, s), 4.729 (2H, s), 6.72-6.82 (2H, m), 8.115 (1H, dd, J=8.4, 9.0 Hz).

Elemental Analysis (C₉H₈NO₅F) Cal'd: C, 47.17; H, 3.52; N, 6.11. Found: C, 47.13; H, 3.30; N, 6.09.

(2) 10% Palladium carbon (0.2 g) and a 4N solution of hydrogen chloride in ethyl acetate (1.5 ml) were added to a solution of methyl 2-[(3-fluoro-4-nitrophenyl)oxy]acetate (1.3 g, 5.67 mmol) obtained in Example 136-(1) in methanol (26 ml), and the mixture was subjected to normal pressure catalytic reduction at room temperature for 2 hours. The catalyst was filtered to remove, and the filtrate was concentrated under reduced pressure. The residue was washed with ethanol-hexane (2:5) to obtain methyl 2-[(4-amino-3-fluorophenyl)oxy]acetate hydrochloride (0.47 g, 1.99 mmol, 35%) as a colorless powder.

m.p. 179-183° C. (dec).

IR νₘₐₓ (KBr) cm⁻¹: 3500-2400 (br, NH₃⁺), 1768, 1757 (C═O).

¹H-NMR (CD₃OD) δ: 3.784 (3H, s), 4.806 (2H, s), 6.87-6.94 (1H, m), 7.029 (1H, d, J=3.0, 12.4 Hz), 7.36-7.46 (1H, m).

Elemental Analysis (C₉H₁₀NO₃F.HCl) Cal'd: C, 45.87; H, 4.71; N, 5.94. Found: C, 45.47; H, 4.59; N, 5.89.

(3) Triethylamine (0.10 g, 1.01 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (0.5 g, 0.962 mmol) obtained in Example 1-(1) in N,N-dimethylformamide solution (2.5 ml) at room temperature. The mixture was ice-cooled, isobutyl chloroformate (0.16 g, 1.15 mmol) was added dropwise over 10 minutes under a nitrogen stream, and the mixture was stirred for 30 minutes under ice-cooling. Methyl 2-[(4-amino-3-fluorophenyl)oxy]acetate hydrochloride (0.25 g, 1.06 mmol) obtained in Example 136-(2) was added, and pyridine (0.12 g, 1.54 mmol) was added dropwise. A temperature was raised to room temperature, the mixture was stirred for 1 hour, water (50 ml) and 1 N hydrochloric acid (2 ml) were added to the reaction solution, and the mixture was extracted with ethyl acetate (50 ml) twice. The whole organic layer was washed with a 5% aqueous potassium hydrogen sulfate solution, an aqueous saturated sodium bicarbonate and an aqueous saturated sodium chloride solution, dried with anhydrous sodium sulfate, and concentrated under reduced pressure. The residue was purified by column chromatography [eluent:hexane-ethyl acetate (1:1)] to obtain methyl 2-[4-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-fluorophenyloxy]acetate (0.30 g, 0.428 mmol, 44%) as a colorless amorphous powder.

[α]_{D}²²−133.4° (c=0.25, MeOH).

IR νₘₐₓ (KBr) cm⁻¹: 3323 (NH), 1738, 1682 (C═O).

¹H-NMR (CDCl₃) δ: 0.954 (3H, s), 1.022 (3H, s), 2.028 (3H, s), 2.839 (1H, dd, J=5.4, 14.2 Hz), 3.042 (1H, dd, J=7.4, 14.2 Hz), 3.544 (1H, d, J=14.2 Hz), 3.619 (3H, s), 3.723 (1H, d, J=11.4 Hz), 3.808 (3H, s), 3.872 (1H, d, J=11.4 Hz), 3.894 (3H, s), 4.403 (1H, dd, J=5.4, 7.4 Hz), 4.577 (1H, d, J=14.2 Hz), 4.601 (2H, s), 6.293 (1H, s), 6.63-6.74 (3H, m), 6.96-7.38 (5H, m), 7.885 (1H, brs), 8.087 (1H, t, J=9.0 Hz).

Elemental Analysis (C₃₅H₃₈N₂O₁₀ClF) Cal'd: C, 59.96; H, 5.46; N, 4.00. Found: C, 60.14; H, 5.71; N, 3.83.

(4) A mixture of methyl 2-[4-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-fluorophenyloxy]acetate (0.2 g, 0.285 mmol) obtained in Example 136-(3), a 1N aqueous sodium hydroxide solution (0.7 ml) and ethanol (3 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:2) to obtain 2-[4-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-fluorophenyloxy]acetic acid (95 mg, 0.147 mmol, 52%) as colorless prisms.

m.p. 192-193° C. (dec).

[α]_{D}²²−143.0° (c=0.23, MeOH).

IR νₘₐₓ (KBr) cm⁻¹: 3600-2400 (br, COOH, OH, NH), 1739, 1653 (C═O).

¹H-NMR (CDCl₃) δ: 0.652 (3H, s), 1.042 (3H, s), 2.855 (1H, dd, J=4.8, 14.4 Hz), 3.068 (1H, dd, J=7.4, 14.4 Hz), 3.204 (1H, d, J=11.8 Hz), 3.391 (1H, d, J=14.6 Hz), 3.614 (3H, s), 3.620 (1H, d, J=11.8 Hz), 3.890 (3H, s), 4.39-4.50 (2H, m), 4.594 (2H, s), 6.178 (1H, s), 6.629 (1H, s), 6.67-6.72 (2H, m), 6.97-7.35 (5H, m), 7.93-8.04 (2H, m).

Elemental Analysis (C₃₂H₃₄N₂O₉ClF) Cal'd: C, 59.58; H, 5.31; N, 4.34. Found: C, 59.46; H, 5.35; N, 4.08.

### EXAMPLE 137

2-[3-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]-2,2-dimethylacetic acid

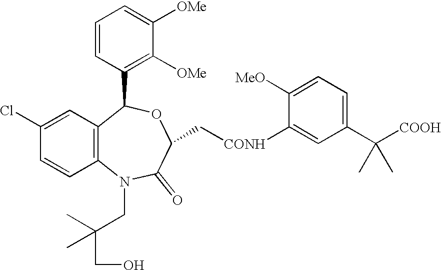

(1) A mixture of 2-(4-hydroxy-3-nitrophenyl)acetic acid (3 g, 15.2 mmol), sodium hydride (1.6 g, 67.0 mmol), iodomethane (8.8 g, 62.0 mmol) and N,N-dimethylformamide (30 ml) was stirred at room temperature for 6 hours. This mixture was diluted with water, and extracted with ethyl acetate (100 ml). The extract was washed with a 5% aqueous potassium hydrogen sulfate solution, and an aqueous saturated sodium bicarbonate solution and an aqueous saturated sodium chloride solution, dried with anhydrous sodium sulfate, and concentrated under reduced pressure. The residue was purified by column chromatography [eluent:ethyl acetate-hexane (1:3)] to obtain methyl 2-(4-methoxy-3-nitrophenyl)-2,2-dimethylacetate (3.3 g, 12.9 mmol, 85%) as a pale yellow oil.

IR νₘₐₓ (KBr) cm⁻¹: 1732 (C═O).

¹H-NMR (CDCl₃) δ: 1.593 (6H, s), 3.667 (3H, s), 3.956 (3H, s), 7.051 (1H, d, J=8.8 Hz), 7.529 (1H, dd, J=2.6, 8.8 Hz), 7.860 (1H, d, J=2.6 Hz).

(2) 10% Palladium carbon (0.1 g) and a 4N solution of hydrogen chloride in ethyl acetate (1 ml) were added to a solution of methyl 2-(4-methoxy-3-nitrophenyl)-2,2-dimethylacetate (1 g, 3.95 mmol) obtained in Example 137-(1) in methanol (20 ml), and the mixture was subjected to normal pressure catalytic reduction at room temperature for 2 hours. The catalyst was filtered to remove, and the filtrate was concentrated under reduced pressure. The residue was washed with ethyl acetate-hexane (1:1) to obtain methyl 2-(3-amino-4-methoxyphenyl)-2,2-dimethylacetate hydrochloride (1.0 g, 3.73 mmol, 95%) as a colorless powder.

m.p. 172-174° C. (dec).

IR νₘₐₓ (KBr) cm⁻¹: 3500-2400 (br, NH₃⁺), 1738 (C═O).

¹H-NMR (CDCl₃) δ: 1.564 (6H, s), 3.643 (3H, s), 3.969 (3H, s), 7.185 (1H, d, J=8.8 Hz), 7.386 (1H, d, J=2.6 Hz), 7.459 (1H, dd, J=2.6, 8.8 Hz).

Elemental Analysis (C₁₂H₁₁NO₃.HCl.0.2H₂O) Cal'd: C, 54.73; H, 7.04; N, 5.32. Found: C, 54.66; H, 6.92; N, 5.23.

(3) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 0.577 mmol) obtained in Example 1-(1) in N,N-dimethylformamide (5 ml) at room temperature. The mixture was ice-cooled, isobutyl chloroformate (0.31 g, 2.30 mmol) was added dropwise for 10 minutes under a nitrogen stream, and the mixture was stirred for 30 minutes under ice-cooling. Methyl 2-(3-amino-4-methoxyphenyl)-2,2-dimethylacetate hydrochloride (0.55 g, 2.11 mmol obtained in Example 137-(2) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and extracted with ethyl acetate (50 ml) twice. The whole organic layer was washed with a 5% potassium hydrogen sulfate solution, an aqueous saturated sodium bicarbonate solution and an aqueous saturated sodium chloride solution, dried with anhydrous sodium sulfate, and concentrated under reduced pressure. The residue was purified by column chromatography [eluent:hexane-ethyl acetate (1:1)] and recrystallization from ethyl acetate-hexane (1:1) to obtain methyl 2-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]-2,2-dimethylacetate (0.69 g, 0.951 mmol, 50%) as a colorless amorphous powder.

[α]_{D}²²−164.8° (c=0.13, MeOH).

IR νₘₐₓ (KBr) cm⁻¹: 3350 (NH), 1732, 1680 (C═O).

¹H-NMR (CDCl₃) δ: 0.954 (3H, s), 1.022 (3H, s), 1.553 (6H, s), 2.027 (3H, s), 2.845 (1H, dd, J=6.2, 14.6 Hz), 3.031 (1H, dd, J=6.6, 14.6 Hz), 3.550 (1H, d, J=13.8 Hz), 3.610 (3H, s), 3.643 (3H, s), 3.721 (1H, d, J=11.4 Hz), 3.782 (3H, s), 3.873 (1H, d, J=11.4 Hz), 3.890 (3H, s), 4.447 (1H, dd, J=6.2, 6.6 Hz), 4.579 (1H, d, J=13.8 Hz), 6.291 (1H, s), 6.637 (1H, s), 6.77-7.34 (7H, m), 8.192 (1H, brs), 8.398 (1H, d, J=2.2 Hz).

Elemental Analysis (C₃₈H₄₅N₂O₁₀Cl) Cal'd: C, 62.93; H, 6.25; N, 3.86. Found: C, 62.70; H, 6.48; N, 3.95.

(4) A mixture of methyl 2-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]-2,2-dimethylacetate (0.58 g, 0.800 mmol) obtained in Example 137-(3), a 1N aqueous sodium hydroxide solution (2 ml) and ethanol (6 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:2) to obtain 2-[3-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]-2,2-dimethylacetic acid (73 mg, 0.109 mmol, 14%) as a colorless powder.

m.p. 225-226° C. (dec).

[α]_{D}²²−169.8° (c=0.15, MeOH).

IR νₘₐₓ (KBr) cm⁻¹: 3600-2400 (br, COOH, OH, NH), 1712, 1687, 1651 (C═O).

¹H-NMR (CDCl₃) δ: 0.665 (3H, s), 1.044 (3H, s), 1.555 (6H, s), 2.844 (1H, dd, J=6.2, 15.4 Hz), 3.059 (1H, dd, J=6.6, 15.4 Hz), 3.147 (1H, d, J=12.6 Hz), 3.414 (1H, d, J=14.8 Hz), 3.606 (3H, s), 3.608 (1H, d, J=12.6 Hz), 3.806 (3H, s), 3.894 (3H, s), 4.41-4.51 (2H, m), 6.187 (1H, s), 6.603 (1H, s), 6.614 (1H, s), 6.79-7.38 (7H, m), 8.209 (1H, s), 8.403 (1H, s).

Elemental Analysis (C₃₅H₄₁N₂O₉Cl.H₂O) Cal'd: C, 61.18; H, 6.31; N, 4.08. Found: C, 60.97; H, 6.04; N, 3.95.

### EXAMPLE 138

4-[3-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]-4,4-dimethylbutanoic acid

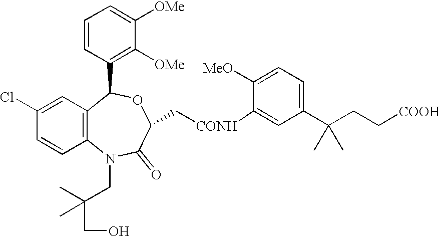

(1) A mixture of methyl 2-(4-methoxy-3-nitrophenyl)-2,2-dimethylacetate (2 g, 7.90 mmol) obtained in Example 137-(1), a 1N aqueous sodium hydroxide solution (20 ml) and ethanol (20 ml) was stirred at 60° C. for 2 hours. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:3) to obtain 2-(4-metoxy-3-nitrophenyl)-2,2-dimethylacetic acid (1.7 g, 7.19 mmol, 91%) as colorless prisms.

m.p. 225-226° C. (dec).

IR νₘₐₓ (KBr) cm⁻¹: 3500-2400 (COOH), 1703 (C═O).

¹H-NMR (CDCl₃) δ: 1.617 (6H, s), 3.956 (3H, s), 7.066 (1H, d, J=8.8 Hz), 7.589 (1H, dd, J=2.6, 8.8 Hz), 7.902 (1H, d, J=2.6 Hz).

Elemental Analysis (C₁₁H₁₃NO₅) Cal'd: C, 55.23; H, 5.48; N, 5.86. Found: C, 55.29; H, 5.35; N, 5.60.

(2) Carbonyldiimidazole (1.2 g, 7.36 mmol) was added to a solution of 2-(4-metoxy-3-nitrophenyl)-2,2-dimethylacetic acid (1.6 g, 6.69 mmol) obtained in Example 138-(1) in tetrahydrofuran (20 ml) at room temperature. The mixture was stirred at room temperature for 1.5 hours, and magnesium chloride (0.64 g, 6.69 mmol) and a potassium salt of malonic acid monoethyl ester (1.1 g, 6.69 mmol) were added. This mixture was stirred at 60° C. for 1 hour, the reaction solution was diluted with ethyl acetate (100 ml), washed with 1N hydrochloric acid, an aqueous saturated sodium bicarbonate solution and an aqueous saturated sodium chloride solution, dried with anhydrous sodium sulfate, and concentrated under reduced pressure. The residue was purified by silica gel column chromatography [eluent:hexane-ethyl acetate (2:1)]to obtain ethyl 4-(4-methoxy-3-nitrophenyl)-4,4-diemthyl-3-oxobutanoate (1.7 g, 5.50 mmol, 82%) as a pale yellow oil.

IR νₘₐₓ (KBr) cm⁻: 1745, 1712 (C═O).

¹H-NMR (CDCl₃): 1.232 (9/10×3H, t, J=7.2 Hz), 1.304 (1/10×3H, t, J=7.2 Hz), 1.533 (9/10×6H, s), 1.544 (1/10×6H, s), 3.293 (9/10×2H, s), 3.954 (1/10×3H, s), 3.972 (9/10×3H, s), 4.125 (9/10×2H, q, J=7.2 Hz), 4.210 (1/10×2H, q, J=7.2 Hz), 5.108 (1/10×1H, s), 7.045 (1/10×1H, d, J=8.8 Hz), 7.099 (9/10×1H, d, J=8.8 Hz), 7.421 (9/10×1H, dd, J=2.6, 8.8 Hz), 7.53 (1/10×1H, dd, J=2.6, 8.8 Hz), 7.816 (9/10×1H, d, J=2.6 Hz), 7.845 (1/10×1H, d, J=2.6 Hz).

(3) Sodium borohydrate (0.20 g, 5.33 mmol) was added to a solution of ethyl 4-(4-methoxy-3-nitrophenyl)-4,4-diemthyl-3-oxobutanoate (1.5 g, 4.85 mmol) obtained in Example 138-(2) in methanol (20 ml) at −20° C. After the mixture was stirred at −20° C. for 30 minutes, and 1N hydrochloric acid (6 ml) was added. This mixture was diluted with ethyl acetate (100 ml), washed with water, an aqueous saturated sodium bicarbonate solution and an aqueous saturated sodium chloride solution, dried with anhydrous sodium sulfate and the residue was purified by silica gel column chromatography [eluent:hexane-ethyl acetate (3:2)] to obtain ethyl 3-hydroxy-4-(4-methoxy-3-nitrophenyl)-4,4-dimethylbutanoate (1.5 g, 4.88 mmol, quant) as a colorless oil.

IR νₘₐₓ (KBr) cm⁻¹: 3600-3300 (br, OH), 1732 (C═O).

¹H-NMR (CDCl₃) δ: 1.240 (3H, t, J=7.4 Hz), 1.370 (6H, s), 2.157 (1H, dd, J=10.2, 16.4 Hz), 2.332 (1H, dd, J=2.6, 16.4 Hz), 3.078 (1H, d, J=3.4 Hz), 3.954 (3H, s), 4.02-4.09 (1H, m), 4.123 (2H, q, J=7.4 Hz), 7.048 (1H, d, J=8.6 Hz), 7.615 (1H, dd, J=2.6, 8.6 Hz), 7.874 (1H, d, J=2.6 Hz).

(4) A mixture of ethyl 3-hydroxy-4-(4-methoxy-3-nitrophenyl)-4,4-dimethylbutanoate (1.4 g, 4.50 mmol) obtained in Example 138-(3), triethylamine (0.55 g, 5.40 mmol), methenesulfonyl chloride (0.57 g, 4.95 mmol) and ethyl acetate (15 ml) was stirred at 0° C. for 30 minutes. 1,8-diazabicyclo[5.4.0]-7-undecene (0.82 g, 5.40 mmol) was added, and this mixture was stirred at 0° C. for 30 minutes. This mixture was stirred with ethyl acetate (50 ml), and washed with 1N hydrochloric acid (11 ml), an aqueous saturated solution of sodium bicarbonate solution and an aqueous saturated sodium chloride solution. The mixture was dried with anhydrous sodium sulfate, and concentrated under reduced pressure. The residue was purified by silica gel column chromatography [eluent: hexane-ethyl acetate (7:3)] to obtain ethyl 4-(4-methoxy-3-nitrophenyl)-4,4-dimethyl-2-butenoate (1.2 g, 3.92 mmol, 79%) as a colorless oil.

IR νₘₐₓ (KBr) cm⁻¹: 1716 (C═O), 1651 (C═C).

¹H-NMR (CDCl₃) δ: 1.299 (3H, t, J=7.4 Hz), 1.476 (6H, s), 3.953 (3H, s), 4.204 (2H, q, J=7.4 Hz), 5.795 (1H, d, J=15.8 Hz), 7.043 (1H, d, J=8.8 Hz), 7.044 (1H, d, J=15.8 Hz), 7.462 (1H, dd, J=2.6, 8.8 Hz), 7.787 (1H, d, J=2.6 Hz).

(5) 10% Palladium carbon (0.1 g) and a 4N solution of hydrogen chloride in ethanol (100 ml) were added to a solution of ethyl 4-(4-methoxy-3-nitrophenyl)-4,4-dimethyl-2-butenoate (1 g, 3.41 mmol) obtained in Example 138-(4) in ethanol (100 ml). This suspension was subjected to normal pressure catalytic reduction at room temperature for 1 hour. The catalyst was filtered to remove, and the filtrate was concentrated under reduced pressure. The residue was washed with hexane to obtain ethyl 4-(3-amino-4-methoxyphenyl)-4,4-dimethylbutanoate hydrochloride (1.1 g, 3.54 mmol, quant) as a brown oil.

IR νₘₐₓ (KBr) cm⁻¹: 3600-2400 (br, NH₃⁺), 1732 (C═O).

¹H-NMR (CD₃OD) δ: 1.183 (3H, t, J=7.0 Hz), 1.321 (6H, s), 1.90-2.10 (4H, m), 3.963 (3H, s), 4.019 (2H, q, J=7.0 Hz), 7.167 (1H, d, J=8.8 Hz), 7.347 (1H, d, J=2.2 Hz), 7.457 (1H, dd, J=2.2, 8.8 Hz).

(6) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepine-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and stirred for 30 minutes under ice-cooling. Ethyl 4-(3-amino-4-methoxyphenyl)-4,4-dimethylbutanoate hydrochloride (0.64 g, 2.11 mmol) obtained in Example 138-(5) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and the mixture was extracted with ethyl acetate (50 ml) twice. The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by column chromatography [eluent:hexane-ethyl acetate (1:1)] and recrystallization from ethyl acetate-hexane (1:1) to obtain methyl 4-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]-4,4-dimethylbutanoate (1.08 g, 1.41 mmol, 73%) as a colorless powder.

m.p. 157-158° C.

[α]_{D}²²−161.3° (c=0.15, MeOH).

IR νₘₐₓ (KBr) cm⁻¹: 3335 (NH), 1732, 1682 (C═O).

¹H-NMR (CDCl₃) δ: 0.956 (3H, s), 1.026 (3H, s), 1.198 (3H, t, J=7.4 Hz), 1.291 (6H, s), 1.89-2.09 (4H, m), 2.029 (3H, s), 2.853 (1H, dd, J=6.2, 14.8 Hz), 3.035 (1H, dd, J=6.6, 14.8 Hz), 3.555 (1H, d, J=14.0 Hz), 3.612 (3H, s), 3.723 (1H, d, J=11.4 Hz), 3.782 (3H, s), 3.873 (1H, d, J=11.4 Hz), 3.888 (3H, s), 4.046 (2H, q, J=7.4 Hz), 4.460 (1H, dd, J=6.2, 6.6 Hz), 4.587 (1H, d, J=14.0 Hz), 6.293 (1H, s), 6.637 (1H, s), 6.76-7.34 (7H, m), 8.156 (1H, brs), 8.350 (1H, d, J=2.2 Hz).

Elemental Analysis (C₄₁H₅₁N₂O₁Cl) Cal'd: C, 64.18; H, 6.70; N, 3.65. Found: C, 63.90; H, 6.65; N, 3.57.

(7) A mixture of methyl 4-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]-4,4-dimethylbutanoate (0.9 g, 1.17 mmol) obtained in Example 138-(6), a 1N aqueous sodium hydroxide solution (3 ml) and ethanol (10 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethyl acetate-hexane (1:1) to obtain 4-[3-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]-4,4-dimethylbutanoic acid (0.70 g, 1.00 mmol, 86%) as a colorless powder.

m.p. 173-174° C.

[α]_{D}²²−171.4° (c=0.15, MeOH).

IR νₘₐₓ (KBr) cm⁻¹: 3600-2400 (br, COOH, OH, NH), 1709, 1658 (C═O).

¹H-NMR (CDCl₃) δ: 0.645 (3H, s), 1.042 (3H, s), 1.297 (6H, s), 1.88-2.14 (4H, m), 2.846 (1H, dd, J=5.8, 14.6 Hz), 3.069 (1H, dd, J=6.8, 14.6 Hz), 3.147 (1H, d, J=11.8 Hz), 3.379 (1H, d, J=14.8 Hz), 3.603 (3H, s), 3.612 (1H, d, J=11.8 Hz), 3.756 (3H, s), 3.890 (3H, s), 4.44-4.51 (2H, m), 6.187 (1H, s), 6.617 (1H, s), 6.76-7.35 (7H, m), 8.227 (1H, brs), 8.324 (1H, d, J=1.8 Hz).

Elemental Analysis (C₃₇H₄₅N₂O₉Cl.0.3H₂O) Cal'd: C, 63.25; H, 6.54; N, 3.99. Found: C, 63.25; H, 6.24; N, 3.98.

### EXAMPLE 139

5-[3-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid

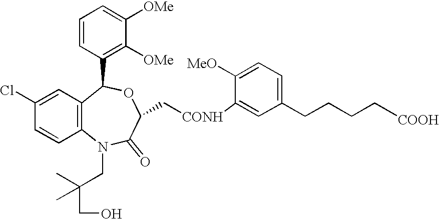

(1) A solution of 4-methoxy-3-nitrobenzaldehyde (1 g, 5.52 mmol) and triethyl 4-phosphonocrotonate (1.4 g, 5.52 mmol) in tetrahydrofuran (30 ml) was added dropwise to a mixture of sodium hydride (0.15 g, 6.07 mmol) and tetrahydrofuran (10 ml) at 0° C. The mixture was stirred at room temperature for 30 minutes, and the reaction was quenched with water. This was diluted with ethyl acetate (50 ml), washed with 1N hydrochloric acid, an aqueous saturated sodium bicarbonate solution and an aqueous saturated sodium chloride solution, dried with anhydrous sodium sulfate, and concentrated under reduced pressure. The residue was purified by recrystallization from ethyl acetate-hexane (1:2) to obtain ethyl 5-(4-methoxy-3-nitrophenyl)pentane-2,4-dieonate (1.12 g, 4.04 mmol, 73%) as yellow prisms.

m.p. 114-116° C.

IR νₘₐₓ (KBr) cm⁻¹: 1699 (C═O), 1608 (C═C).

¹H-NMR (CDCl₃) δ: 1.324 (3H, t, J=7.0 Hz), 3.995 (3H, s), 4.240 (2H, q, J=7.0 Hz), 6.020 (1H, d, J=15.0 Hz), 6.822 (2H, d, J=5.4 Hz), 7.086 (1H, d, J=8.8 Hz), 7.420 (1H, dt, J=15.0, 5.4 Hz), 7.624 (1H, dd, J=2.2, 8.8 Hz), 7.814 (1H, d, J=2.2 Hz). Elemental Analysis (C₁₄H₂₁NO₃.HCl) Cal'd: C, 60.64; H, 5.45; N, 5.05. Found: C, 60.62; H, 5.40; N, 4.97.

(2) 10% Palladium carbon (0.1 g) and a 4N solution of hydrogen chloride in ethyl acetate (1 ml) were added to a solution of ethyl 5-(4-methoxy-3-nitrophenyl)pentane-2,4-dieonate (0.9 g, 3.25 mmol) obtained in Example 139-(1) in ethanol (20 ml), and the mixture was subjected to normal pressure catalytic reduction at room temperature. The catalyst was filtered to remove, and the filtrate was concentrated under reduced pressure. The residue was washed with ethyl acetate-hexane (1:1) to obtain ethyl 5-(3-amino-4-methoxyphenyl)pentanoate hydrochloride (0.87 g, 3.02 mmol, 93%) as a colorless powder.

m.p. 157-158° C. (dec).

IR νₘₐₓ (KBr) cm⁻¹: 3200-2400 (br, NH₃⁺), 1730 (C═O).

¹H-NMR (CD₃OD): 1.225 (3H, t, J=7.4 Hz), 1.59-1.66 (4H, m), 2.30-2.37 (2H, m), 2.59-2.66 (2H, m), 3.947 (3H, s), 4.099 (2H, q, J=7.4 Hz), 7.123 (1H, d, J=8.8 Hz), 7.187 (1H, d, J=2.2 Hz), 7.285 (1H, dd, J=2.2, 8.8 Hz).

(3) Triethylamine (0.20 g, 2.02 mmol) was added to a solution of (3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-acetic acid (1 g, 1.92 mmol) obtained in Example 1-(1) in N,N-dimethylformamide (5 ml) at room temperature. The mixture was ice-cooled, isobutyl chloroformate (0.31 g, 2.30 mmol) was added dropwise over 10 minutes under a nitrogen stream, and the mixture was stirred for 30 minutes under ice-cooling. Ethyl 5-(3-amino-4-methoxyphenyl)pentanoate hydrochloride (0.36 g, 2.11 mmol) obtained in Example 139-(2) was added, and pyridine (0.24 g, 3.07 mmol) was added dropwise. A temperature was raised to room temperature, the mixture was stirred for 1 hour, water (50 ml) and 1N hydrochloric acid (4 ml) were added to the reaction solution, and the mixture was extracted with ethyl acetate (50 ml). The whole organic layer was washed with a 5% aqueous potassium hydrogen sulfate solution, an aqueous saturated sodium bicarbonate solution and an aqueous saturated sodium chloride solution, dried with anhydrous sodium sulfate, and concentrated under reduced pressure. The residue was purified by column chromatography [eluent:hexane-ethyl acetate (3:2)] to obtain ethyl 5-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]pentanoate (1.1 g, 1.47 mmol, 77%) as a colorless amorphous powder.

[α]_{D}²²−159.0° (c=0.38, MeOH).

IR νₘₐₓ (KBr) cm⁻¹: 3341 (NH), 1736, 1682 (C═O).

¹H-NMR (CDCl₃) δ: 0.954 (3H, s), 1.022 (3H, s), 1.238 (3H, t, J=7.4 Hz), 1.56-1.68 (4H, m), 2.028 (3H, s), 2.26-2.33 (2H, m), 2.52-2.59 (2H, m), 2.853 (1H, dd, J=5.8, 14.6 Hz), 3.034 (1H, dd, J=6.6, 14.6 Hz), 3.544 (1H, d, J=14.0 Hz), 3.610 (3H, s), 3.723 (1H, d, J=11.4 Hz), 3.775 (3H, s), 3.872 (1H, d, J=11.4 Hz), 3.890 (3H, s), 4.109 (2H, q, J=7.4 Hz), 4.454 (1H, dd, J=5.8, 6.6 Hz), 4.580 (1H, dd, J=14.0 Hz), 6.293 (1H, s), 6.638 (1H, s), 6.76-7.33 (7H, m), 8.169 (2H, brs).

Elemental Analysis (C₄₀H₄₉N₂O₁₀Cl) Cal'd: C, 63.78; H, 6.56; N, 3.72. Found: C, 63.69; H, 6.55; N, 3.61.

(4) A mixture of ethyl 5-[3-[[2-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methoxyphenyl]pentanoate (1 g, 1.33 mmol) obtained in Example 139-(3), a 1N aqueous sodium hydroxide solution (3 ml) and ethanol (10 ml) was stirred at 60° C. for 30 minutes. This was diluted with water (50 ml), acidified, and extracted with ethyl acetate (100 ml). This was washed with an aqueous saturated sodium chloride solution, dried with anhydrous sodium sulfate, and concentrated under reduced pressure. The residue was purified by recrystallization from ethanol-hexane (1:1) to obtain 5-[3-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid (0.69 g, 1.01 mmol, 76%) as colorless needles.

m.p. 136-138° C.

[α]_{D}²²−178.5° (c=0.25, MeOH).

IR νₘₐₓ (KBr) cm⁻¹: 3600-2400 (br, COOH, OH, NH), 1705, 1660 (C═O).

¹H-NMR (CDCl₃) δ: 0.652 (3H, s), 1.051 (3H, s), 1.61-1.68 (4H, m), 2.32-2.36 (2H, m), 2.54-2.58 (2H, m), 2.858 (1H, dd, J=5.8, 15.0 Hz), 3.073 (1H, dd, J=6.6, 15.0 Hz), 3.160 (1H, d, J=12.6 Hz), 3.390 (1H, d, J=14.0 Hz), 3.608 (3H, s), 3.628 (1H, d, J=12.6 Hz), 3.789 (3H, s), 3.892 (3H, s), 4.43-4.52 (2H, m), 6.189 (1H, s), 6.617 (1H, s), 6.74-7.36 (7H, m), 8.15-8.18 (2H, m).

Elemental Analysis (C₃₆H₄₃N₂O₉Cl.0.5H₂O) Cal'd: C, 62.47; H, 6.41; N, 4.05. Found: C, 62.22; H, 6.30; N, 3.75.

### EXAMPLE 140

6-[[[(3R,5S)-5-(2,3-Dimethoxyphenyl)-7-chloro-1-(3-hydroxy-2,2-dimethylpropyl)-2-oxo-1,2,3,5-tetrahydro-4,1-benzoxazepin-3-yl]acetyl]amino]-2-pyridinecarboxylic acid

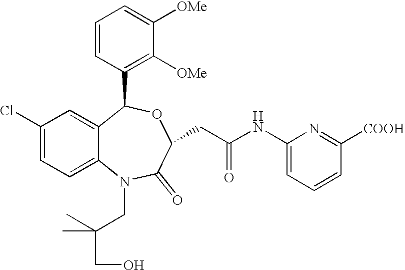

(1) (3R,5S)-1-(3-acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in tetrahydrofuran (10 ml), and one droplet of N,N-dimethylformamide was added. Thionyl chloride (0.17 ml, 2.31 mmol) was added under ice-cooling, a temperature was raised to room temperature, the mixture was stirred for 3 hours, concentrated under reduced pressure, and dissolved in tetrahydrofuran (9 ml). Ethyl 6-amino-2-pyridinecarboxylate (0.32 g, 1.92 mmol) was dissolved in tetrahydrofuran (5 ml), and triethylamine (0.29 ml, 2.12 mmol) was added. The previously prepared acid chloride solution was added dropwise at room temperature, and the mixture was stirred at the same temperature for 1.5 hours. Water and ethyl acetate were added to the reaction solution, the layers were separated, and the organic layer was washed with water and an aqueous saturated sodium chloride solution. This was dried with anhydrous sodium sulfate, and concentrated under reduced pressure. The resulting residue was purified by silica gel column chromatography (hexane:ethyl acetate=2:1) to obtain ethyl 6-[[[(3R,5S)-1-(3-acetoxy-2,2-dimethylpropyl)-5-(2,3-dimethoxyphenyl)-7-chloro-2-oxo-1,2,3,5-tetrahydro-4,1-benzoxazepin-3-yl]acetyl]amino]-2-pyridinecarboxylate (0.8 g, yield 63.6%) as a colorless foam.

[α]_{D}²²−159.9° (c=0.40, methanol).

¹H-NMR (200 MHz, CDCl₃) δ: 0.95 (3H, s), 1.03 (3H, s), 1.43 (3H, t, J=7.2 Hz), 2.03 (3H, s), 2.90 (1H, dd, J=15.0, 7.0 Hz), 3.55 (1H, d, J=13.8 Hz), 3.63 (3H, s), 3.73 (1H, d, J=13.8 Hz), 3.80 (1H, d, J=14.4 Hz), 3.89 (3H, s), 4.46 (2H, q, J=7.0 Hz), 4.45-4.53 (1H, m), 4.59 (1H, d, J=14.4 Hz), 6.30 (1H, s), 6.65 (1H, d, J=0.8 Hz), 6.98 (1H, dd, J=7.4, 2.2 Hz), 8.57 (1H, brs).

IR (KBr) 3268, 2965, 2940, 1734, 1682, 1578, 1537, 1456 cm⁻¹.

Elemental Analysis (C₃₄H₃₈N₃O₉Cl.0.5H₂O) Cal'd: C, 60.31; H, 5.81; N, 6.21. Found: C, 60.39; H, 5.78; N, 6.09.

(2) Ethyl 6-[[[(3R,5S)-1-(3-acetoxy-2,2-dimethylpropyl)-5-(2,3-dimethoxyphenyl)-7-chloro-2-oxo-1,2,3,5-tetrahydro-4,1-benzoxazepin-3-yl]acetyl]amino]-2-pyridinecarboxylate (0.71 g, 1.09 mmol) obtained in Example 140 (1) was dissolved in tetrahydrofuran (4 ml) and ethanol (2 ml), a 1N aqueous sodium hydroxide solution (1 ml) was added at room temperature, and the solution was stirred at the same temperature for 30 minutes. The solution was neutralized using 1N hydrochloric acid, and extracted with chloroform. The organic layer was washed with an aqueous saturated sodium chloride solution, dried with anhydrous sodium chloride, and concentrated under reduced pressure. The resulting residue was purified by silica gel column chromatography (10% solution of methanol in chloroform) to obtain 6-[[[(3R,5S)-5-(2,3-dimethoxyphenyl)-7-chloro-1-(3-hydroxy-2,2-dimethylpropyl)-2-oxo-1,2,3,5-tetrahydro-5,1-benzoxazepin-3-yl]acetyl]amino]-2-pyridinecarboxylic acid (0.18 g, yield 27.7%) as white crystals.

m.p. 265.0-270.0° C. (dec).

[α]_{D}²²−125.7° (c=0.26, methanol).

¹H-NMR (200 MHz, CD₃OD) δ: 0.82 (3H, s), 0.90 (3H, s), 2.90-3.10 (2H, m), 3.17 (1H, d, J=11.0 Hz), 3.39 (1H, d, J=11.0 Hz), 3.56 (3H, s), 3.63 (1H, d, J=13.8 Hz), 3.86 (3H, s), 4.26-4.40 (2H, m), 6.14 (1H, s), 6.46 (1H, d, J=1.8 Hz), 7.07 (3H, s), 7.35 (1H, brs), 7.74-7.59 (2H, m), 7.77-7.85 (2H, m).

IR (KBr) 3600-2500, 1730-1600, 1481, 1379 cm⁻¹.

Elemental Analysis (C₃₀H₃₂N₃O₈Cl.1.8H₂O) Cal'd: C, 57.15; H, 5.69; N, 6.66. Found: C, 57.10; H, 5.40; N, 6.45.

### EXAMPLE 141

2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazole-5-carboxylic acid

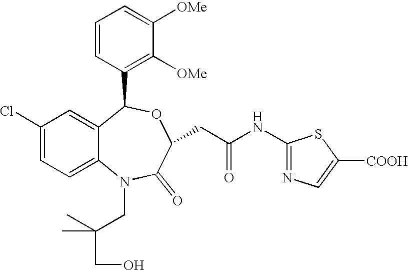

(1) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (5 ml) under the argon atmosphere. Triethylamine (0.21 ml, 1.96 mmol) and isobutyl chloroformate (0.28 ml, 2.22 mmol) were added under ice-cooling, and the mixture was stirred at the same temperature for 30 minutes. A solution of ethyl 2-amino-1,3-thiazole-5-carboxylate in N,N-dimethylformamide (5 ml) was added dropwise, and pyridine (0.25 ml, 3.08 mmol) was added dropwise. The mixture was stirred at the same temperature for 2 hours and at room temperature for 2 hours, water was added to the reaction solution, and the mixture was extracted with ethyl acetate. The organic layer was washed with 1N hydrochloric acid, water and an aqueous sodium chloride solution. This was dried with anhydrous sodium sulfate, concentrated under reduced pressure, and the resulting residue was purified by silica gel column chromatography (hexane:ethyl acetate=2:1) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazole-5-carboxylate (0.81 g, yield 62.1%) as a colorless foam.

[α]_{D}²²−77.6° (c=0.26, methanol).

¹H-NMR (200 MHz, CDCl₃) δ: 0.95 (3H, s), 1.02 (3H, s), 1.35 (3H, t, J=7.0 Hz), 2.02 (3H, s), 3.00 (1H, dd, J=14.6, 6.0 Hz), 3.17 (1H, dd, J=14.6, 7.0 Hz), 3.56 (1H, d, J=14.0 Hz), 3.62 (3H, s), 3.72 (1H, d, J=11.0 Hz), 3.87 (1H, d, J=11.0 Hz), 3.89 (3H, s), 4.33 (2H, q, J=7.0 Hz), 4.41-4.51 (1H, m), 4.59 (1H, d, J=14.0 Hz), 6.30 (1H, s), 6.36 (1H, d, J=1.4 Hz), 6.93-7.01 (1H, m), 7.15 (1H, s), 7.16 (1H, d, J=4.8 Hz), 7.33-7.42 (2H, m), 8.02 (1H, brs), 8.13 (1H, s).

IR (KBr) 3300-2700, 1734, 1709, 1678, 1481, 1287 cm⁻¹.

Elemental Analysis (C₃₂H₃₆N₃O₉ClS.0.2H₂O) Cal'd: C, 56.71; H, 5.41; N, 6.20. Found: C, 56.61; H, 5.35; N, 6.29.

(2) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazole-5-carboxylate (0.61 g, 0.90 mmol) obtained in Example 141-(1) was dissolved in tetrahydrofuran (8 ml) and ethanol (4 ml), a 2N aqueous sodium hydroxide solution (3.69 ml) was added at room temperature, and the mixture was stirred at 40° C. for 2 hours. After allowing to cool, the mixture was neutralized using 1N hydrochloric acid, the mixture was stirred at room temperature for 2 hours, and water (3 ml) was further added, followed by stirring for 1 hour. The crystals were filtered off, washed with ethyl acetate:hexane (1:5), and dried under reduced pressure (50° C.) to obtain 2-[[[(3R,5S)-7-chloro-5-(2,3-dimethoxyphenyl)-1(3-hydroxy-2,2-dimethylpropyl)-2-oxo-1,2,3,5-tetrahydro-4,1-benzoxazepin-3-yl]acetyl]amino]-1,3-thiazole-5-carboxylic acid (0.48 g, yield 87.6%) as white crystals.

m.p. 241.0-242.2° C.

[α]_{D}²²=−84.8° (c=0.20, methanol).

¹H-NMR (200 MHz, DMSO-d₆) δ: 0.77 (3H, s), 0.86 (3H, s), 2.97-3.20 (4H, m), 3.52 (3H, s), 3.69 (1H, d, J=14.6 Hz), 3.84 (3H, s), 4.28-4.43 (2H, m), 4.56 (1H, brs), 6.10 (1H, s), 6.40 (1H, d, J=2.6 Hz), 7.00-7.05 (1H, m), 7.10-7.20 (2H, m), 7.58 (1H, dd, J=8.8, 2.6 Hz), 7.75 (1H, d, J=8.8 Hz), 8.05 (1H, s).

IR (KBr) 3439, 3300-2200, 1703, 1655, 1481 cm⁻¹.

Elemental Analysis (C₂₈H₃₀N₃O₈SCl) Cal'd: C, 54.06; H, 5.18; N, 6.75. Found: C, 54.17; H, 5.10; N, 6.72.

### EXAMPLE 142

2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazole-4-carboxylic acid

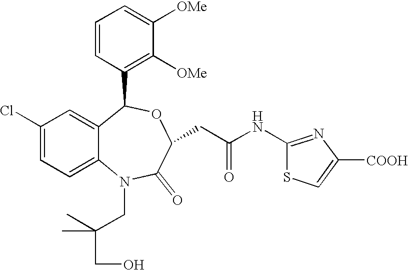

(1) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepin-3-acetic acid (0.5 g, 0.96 mmol) obtained in Example 1-(1) was dissolved in N,N-dimethylformamide (2.5 ml) under the argon atmosphere. Triethylamine (0.14 ml, 0.98 mmol) and isobutyl chloroformate (0.14 ml, 1.11 mmol) were added under ice-cooling, and the mixture was stirred at the same temperature for 30 minutes. A solution of ethyl 2-amino-1,3-thiazole-4-carboxylate (0.17 g, 0.96 mmol) in N,N-dimethylfromamide (2.5 ml) was added dropwise, and pyridine (0.13 ml, 1.53 mmol) was added dropwise. The mixture was stirred at the same temperature for 2 hours and at room temperature for 2 hours, water was added to the reaction solution, and extracted with ethyl acetate. The organic layer was washed with 1N hydrochloric acid, water and an aqueous saturated sodium chloride solution. This was dried with anhydrous sodium sulfate, concentrated under reduced pressure, and the resulting residue was purified by silica gel column chromatography (hexane:ethyl acetate=1:1) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazole-4-carboxylate (84 mg, yield 13.0%) as a colorless foam.

[α]_{D}²²−138.7° (c=0.14, methanol).

¹H-NMR (200 MHz, CDCl₃) δ: 0.94 (3H s), 1.01 (3H, s), 1.39 (3H, t, J=7.4 Hz), 2.03 (3H, s), 2.98 (1H, dd, J=15.4, 5.8 Hz), 3.16 (1H, dd, J=15.4, 6.8 Hz), 3.55 (1H, d, J=14.2 Hz), 3.62 (3H, s), 3.72 (1H, d, J=11.0 Hz), 3.88 (1H, d, J=11.0 Hz), 3.89 (3H, s), 4.30-4.50 (3H, m), 4.59 (1H, d, J=14.2 Hz), 6.29 (1H, s), 6.65 (1H, d, J=2.0 Hz) 6.90-7.01 (1H, m), 7.10-7.21 (2H, m), 7.30-7.40 (2H, m), 7.81 (1H, s).

IR (KBr) 3300-2600, 1732, 1682, 1549, 1481 cm⁻¹.

Elemental Analysis (C₃₂H₃₆N₃O₉ClS.0.2H₂O) Cal'd: C, 56.71; H, 5.41; N, 6.20. Found: C, 56.64; H, 5.48; N, 6.21.

(2) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazole-4-carboxylate (0.16 g, 0.24 mmol) obtained in Example 142-(1) was dissolved in tetrahydrofuran (2 ml) and ethanol (1 ml), a 2N aqueous sodium hydroxide solution (0.47 ml) was added at room temperature, and the mixture was stirred at 45° C. for 3 hours. After allowing to cool, the mixture was neutralized using 1N hydrochloric acid, and water (1 ml) was added, followed by stirring for 1 hour. The crystals were filtered off, washed with ethyl acetate:hexane (1:2), and dried under reduced pressure (50° C.) to obtain 2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazole-4-carboxylic acid (0.11 g, yield 79.3%) as white crystals.

m.p. 277.3-277.9° C.

[α]_{D}²²=−155.8° (c=0.10, methanol).

¹H-NMR (200 MHz, DMSO-d₆) δ: 0.76 (3H, s), 0.85 (3H, s), 2.90-3.01 (2H, m), 3.03-3.20 (2H, m), 3.52 (3H, s), 3.68 (1H, d, J=14.6 Hz), 3.84 (3H, s), 4.32 (1H, d, J=14.8 Hz), 4.39 (1H, t, J=7.2 Hz), 4.56 (1H, brs), 6.10 (1H, s), 6.40 (1H, d, J=2.2 Hz), 7.00-7.01 (1H, m), 7.13-7.20 (2H, m), 7.57 (1H, dd, J=8.8, 2.6 Hz), 7.75 (1H, d, J=8.8 Hz), 7.96 (1H, s).

IR (KBr) 3600-2200, 1680, 1549, 1481 cm⁻¹.

Elemental Analysis (C₂₈H₃₀N₃O₈ClS.0.2H₂O) Cal'd: C, 55.34; H, 5.04; N, 6.91. Found: C, 55.72; H, 4.94; N, 6.54.

### EXAMPLE 143

[2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazol-5-yl]acetic acid

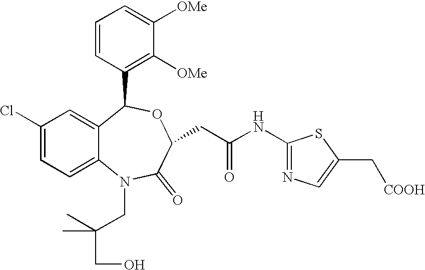

(1) Monoethylsuccinic chloride (10 g, 60.76 mmol) and 2,6-lutidine (7.08 ml, 60.76 mmol) were dissolved in tetrahydrofuran (200 ml), and nitrogen replacement was performed. 10% palladium carbon (750 mg) was added, and hydrogen was introduced (4.0 kgf/cm²). The mixture was stirred at room temperature for 3 days. The catalyst and the insolubles were filtered, and concentrated under reduced pressure. The resulting residue was purified by silica gel column chromatography (hexane:ethyl acetate=4:1) to obtain ethyl 4-oxobutanoate (2.84 g, yield 35.9%) as a colorless oil.

¹H-NMR (200 MHz, CDCl₃) δ: 1.27 (3H, t, J=7.4 Hz), 2.58 -2.70 (2H, m), 2.75-2.86 (2H, m), 2.45 (2H, q, J=7.4 Hz), 9.82 (1H, t, J=0.6 Hz).

IR (KBr) 2984, 1734, 1182 cm⁻¹.

Elemental Analysis (C₆H₁₀O₃.0.2H₂O) Cal'd: C, 53.88; H, 7.84. Found: C, 53.69; H, 7.54.

(2) Ethyl 4-oxobutanoate (2.6 g, 19.98 mmol) obtained in Example 143-(1) was dissolved in dioxane (20 ml), and a solution of bromine (1.02 ml, 19.98 mmol) in dioxane (20 ml) and diethyl ether (20 ml) was added dropwise at room temperature. After stirred for 15 minutes, water and diethyl ether were added, the layers were separated, and the organic layer was washed with an aqueous saturated sodium chloride solution. This was dried with anhydrous sodium sulfate, and concentrated under reduced pressure to obtain a pale brown oil (3.7 g). Subsequently, this oil and thiourea (1.35 g, 17.70 mmol) were dissolved in ethanol (30 ml). The solution was stirred at 80° C. for 1 hour, concentrated under reduced pressure, water and diethyl ether were added, and the layer were separated. A 25% aqueous ammonia solution was added to the aqueous layer, which was extracted with ethyl acetate. The organic layer was washed with water and an aqueous saturated sodium chloride solution. The organic layer was dried with anhydrous sodium sulfate, and concentrated under reduced pressure. The resulting residue was dissolved in ethyl acetate, and a 4N hydrogen chloride-ethyl acetate (5 ml) was added dropwise. After stirred at room temperature for 30 minutes, the crystals were filtered off, and dried under reduced pressure to obtain ethyl 2-(2-amino-1,3-thiazol-5-yl)acetate hydrochloride (3.22 g, yield 72.4% (2 steps)) as pale yellow crystals.

m.p. 129.4-130.0° C.

¹H-NMR (200 MHz, DMSO-d₆) δ: 1.21 (3H, t, J=7.4 Hz), 3.84 (2H, s), 4.12 (2H, q, J=7.4 Hz), 7.16 (1H, s), 9.30 (2H, brs).

IR (KBr) 3400-2200, 1717, 1622, 1190 cm⁻¹.

Elemental Analysis (C₇H₁₁N₂O₂SCl.0.1H₂O) Cal'd: C, 37.45; H, 5.03; N, 12.48. Found: C, 37.35; H, 5.18; N, 12.57.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.5 g, 2.89 mmol) obtained in Example 1-(1) was dissolved in N,N-dimethylformamide (15 ml) under the argon atmosphere. Triethylamine (0.41 ml, 2.94 mmol) and isobutyl chloroformate (0.43 ml, 3.32 mmol) were added under ice-cooling, and the mixture was stirred at same temperature for 30 minutes. Ethyl 2-(2-amino-1,3-thiazol-5-yl)acetate hydrochloride (0.64 g, 2.89 mmol) obtained in Example 143-(2), and pyridine (0.37 mmol, 4.62 mmol) was added dropwise. The mixture was stirred at the same temperature for 2 hours, and stirred at room temperature for 13 hours. Water was added to the reaction solution, followed by extraction with ethyl acetate. The organic layer was washed with 1N hydrochloric acid, water and an aqueous saturated sodium chloride solution. The organic layer was dried with anhydrous sodium sulfate, concentrated under reduced pressure, and the resulting residue was purified by silica gel column chromatography (hexane:ethyl acetate=1:2) to obtain ethyl [2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazol-5-yl]acetate (1.77 g, yield 89.2%) as a colorless foam.

[α]_{D}²²=−105.4° (c=0.20, methanol).

¹H-NMR (200 MHz, CDCl₃) δ: 0.95 (3H, s), 1.01 (3H, s), 1.26 (3H, t, J=7.0 Hz), 2.02 (3H, s), 2.96 (1H, dd, J=15.0, 5.8 Hz), 3.18 (1H, dd, J=15.0, 7.4 Hz), 3.54 (1H, d, J=13.8 Hz), 3.61 (3H, s), 3.72 (1H, d, J=11.4 Hz), 3.76 (2H, s), 3.87 (1H, d, J=11.4 Hz), 3.88 (3H, s), 4.17 (2H, q, J=7.0 Hz), 4.46-4.54 (1H, m), 4.58 (1H, d, J=13.8 Hz), 6.29 (1H, s), 6.64 (1H, brs), 6.93-7.01 (1H, m), 7.10-7.20 (2H, m), 7.27-7.40 (3H, m).

IR (KBr) 2967, 1736, 1678, 1481 cm⁻¹.

Elemental Analysis (C₃₃H₃₈N₃O₉SCl.0.2H₂O) Cal'd: C, 57.29; H, 5.59; N, 6.07. Found: C, 57.28; H, 5.77; N, 6.02.

(4) Ethyl [2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazol-5-yl]acetate (1.5 g, 2.18 mmol) obtained in Example 143-(3) was dissolved in ethanol (30 ml), a 2N aqueous sodium hydroxide solution (3.3 ml) was added at room temperature. The mixture was stirred at room temperature for 2 hours. A 1N hydrochloric acid was added to adjust the mixture to acidic, which was extracted with ethyl acetate and tetrahydrofuran, and the organic layer was washed with an aqueous saturated sodium chloride solution. The organic layer was dried with anhydrous sodium sulfate, and concentrated under reduced pressure. The crude crystals were suspended in ethanol (25 ml) and water (10 ml), and a 1N aqueous sodium hydroxide solution (2.5 ml) was added. Subsequently, 1N hydrochloric acid was added to adjust the mixture to acidic, the mixture was stirred at room temperature for 13 hours. The crystals were filtered off, washed with a 50% aqueous ethanol solution, and dried under reduced pressure to obtain [2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazol-5-yl]acetyl acid (1.13 g, yield 83.9%) as white crystals.

m.p. 239.0-241.0° C.

[α]_{D}²²=−112.3° (c=0.07, methanol).

¹H-NMR (200 MHz, DMSO-d₆) δ: 0.75 (3H, s), 0.85 (3H, s), 2.89-3.00 (2H, m), 3.01-3.21 (2H, m), 3.68 (1H, d, J=13.8 Hz), 3.77 (2H, s), 3.84 (3H, s), 4.31 (1H, d, J=13.8 Hz), 4.36 (1H, t, J=6.6 Hz), 4.54 (1H, brs), 6.09 (1H, s), 6.39 (1H, d, J=2.2 Hz), 7.00-7.20 (3H, m), 7.23 (1H, s), 7.56 (1H, dd, J=8.8, 2.2 Hz), 7.75 (1H, d, J=8.8 Hz).

IR (KBr) 3465, 3400-2500, 1655, 1481, 1292, 1069 cm⁻¹.

Elemental Analysis (C₂₉H₃₂N₃O₈SCl.0.2H₂O) Cal'd: C, 56.03; H, 5.25; N, 6.76. Found: C, 55.85; H, 5.54; N, 6.67.

### EXAMPLE 144

3-[2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazol-5-yl]propionic Acid

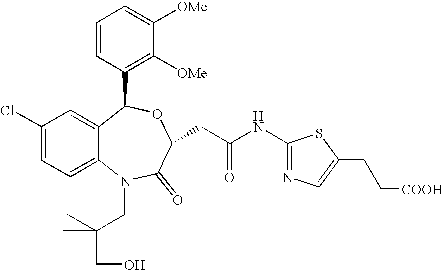

(1) Monoethylglutaric chloride (10 g, 55.99 mmol) and 2,6-lutidine (6.52 ml, 55.99 mmol) were dissolved in tetrahydrofuran (200 ml), and nitrogen replacement was performed. 10% palladium carbon (1.0 g) was added, and hydrogen was introduced (4.0 kgf/cm²). The mixture was stirred at 35° C. for 10 hours. The catalyst and the insolubles were filtered, and concentrated under reduced pressure. The resulting residue was purified by silica gel column chromatography (hexane:ethyl acetate=5:1) to obtain ethyl 5-oxopentanoate (4.8 g, yield 59.5%) as a colorless oil.

¹H-NMR (200 MHz, CDCl₃): 1.26 (3H, t, J=7.2 Hz), 1.96 (2H, m), 2.37 (2H, t, J=7.2 Hz), 2.54 (2H, dd, J=7.2, 1.5 Hz), 4.14 (2H, q, J=7.2 Hz), 9.78 (1H, t, J=1.5 Hz).

IR (KBr) 2984, 1732, 1163 cm⁻¹.

(2) Ethyl 5-oxopentanoate (3.0 g, 20.81 mmol) obtained in Example 144-(1) was dissolved in dioxane (20 ml), and a solution of bromine (1.07 ml, 20.81 mmol) in dioxane (20 ml) and diethyl ether (20 ml) was added dropwise at room temperature. The mixture was stirred for 15 minutes, water and diethyl ether were added, the layers were separated, and the organic layer was washed with an aqueous saturated sodium chloride solution. The organic layer was dried with anhydrous sodium sulfate, and concentrated under reduced pressure to obtain a pale brown oil (4.5 g). Subsequently, this oil and thiourea (1.53 g, 20.17 mmol) were dissolved in ethanol (40 ml). The solution was stirred at 80° C. for 1 hour, concentrated under reduced pressure, water and diethyl ether were added and the layers were separated. A 25% aqueous ammonia solution was added to the aqueous layer, followed by extraction with ethyl acetate. The organic layer was washed with water and an aqueous saturated sodium chloride solution. The organic layer was dried with anhydrous sodium sulfate, and concentrated under reduced pressure. The resulting residue was dissolved in ethyl acetate, a 4N hydrogen chloride-ethyl acetate solution (5 ml) was added dropwise. Concentration under reduced pressure afforded ethyl 3-(2-amino-1,3-thiazol-5-yl)propionate hydrochloride (4.11 g, yield 83.4% (2 steps)) as a pale yellow oil.

¹H-NMR (200 MHz, CDCl₃) δ: 1.27 (3H, t, J=7.2 Hz), 2.61 (2H, t, J=6.6 Hz), 2.93 (2H, t, J=6.6 Hz), 4.16 (2H, q, J=7.2 Hz), 6.83 (1H, s), 9.07 (2H, brs).

IR (KBr) 3700-2300, 1728, 1628, 1568 cm⁻¹.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.5 g, 2.89 mmol) obtained in Example 1-(1) was dissolved in N,N-dimethylformamide (15 ml) under the argon atmosphere. Triethylamine (0.41 ml, 2.94 mmol) and isobutyl chloroformate (0.43 ml, 3.32 mmol) were added under ice-cooling, and the mixture was stirred at the same temperature for 30 minutes. Ethyl 3-(2-amino-1,3-thiazol-5-yl)propionate hydrochloride (0.68 g, 2.89 mmol) obtained in Example 144-(2) was added, pyridine (0.37 ml, 4.62 mmol) was added dropwise. The mixture was stirred at the same temperature for 2 hours, and stirred at room temperature for 3 hours. Water was added to the reaction solution, and followed by extraction with ethyl acetate. The organic layer was washed with 1N hydrochloric acid, water and an aqueous saturated sodium chloride solution. The organic layer was dried with anhydrous sodium sulfate, concentrated under reduced pressure, and the resulting residue was purified by silica gel column chromatography (hexane:ethyl acetate=1:2) to obtain ethyl 3-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazol-5-yl]propionate (1.65 g, yield 81.5%) as a colorless foam.

[α]_{D}²²=−102.0° (c=0.15, methanol).

¹H-NMR (200 MHz, CDCl₃) δ: 0.95 (3H, s), 1.02 (3H, s), 1.24 (3H, t, J=7.0 Hz), 2.02 (3H, s), 2.62 (2H, t, J=8.2 Hz), 2.85-3.22 (4H, m), 3.54 (1H, d, J=14.2 Hz), 3.61 (3H, s), 3.72 (1H, d, J=11.4 Hz), 3.86 (1H, d, J=11.4 Hz), 3.88 (3H, s), 4.13 (2H, q, J=7.0 Hz), 4.48 (1H, t, J=7.0 Hz), 4.58 (1H, d, J=14.2 Hz), 6.28 (1H, s), 6.65 (1H, d, J=1.4 Hz), 6.90-7.01 (1H, m), 7.10-7.21 (3H, m), 7.32-7.40 (2H, m).

IR (KBr) 2965, 1734, 1676, 1481 cm⁻¹.

Elemental Analysis (C₃₄H₄₀N₃O₉SCl) Cal'd: C, 58.15; H, 5.74; N, 5.98. Found: C, 57.89; H, 5.96; N, 5.94.

(4) Ethyl 3-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1,3-thiazol-5-yl]propionate (1.0 g, 1.42 mmol) obtained in Example 144-(3) was dissolved in ethanol (20 ml), a 1N aqueous sodium hydroxide solution (4.3 ml) was added. The mixture was stirred at room temperature for 7 hours. 1N hydrochloric acid to adjust the mixture to acidic, the mixture was stirred at room temperature for 1 hour, the crystals were filtered off, and washed with a 50% aqueous ethanol solution. Drying under reduced pressure afforded 3-[2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1,3-thiazol-5-yl]propionic acid (0.71 g, yield 78.3%) as white crystals.

m.p. 203.0-205.0° C.

[α]_{D}²²=−117.9° (c=0.12, methanol).

¹H-NMR (200 MHz, DMSO-d₆) δ: 0.75 (3H, s), 0.85 (3H, s), 2.55 (2H, t, J=7.0 Hz), 2.80-3.00 (4H, m), 3.01-3.20 (2H, m), 3.51 (3H, s), 3.68 (1H, d, J=13.8 Hz), 3.83 (3H, s), 4.26-4.40 (2H, m), 4.54 (1H, brs), 6.09 (1H, s), 6.39 (1H, d, J=2.2 Hz), 7.00-7.06 (1H, m), 7.07-7.23 (3H, m), 7.56 (1H, dd, J=8.8, 2.2 Hz), 7.76 (1H, d, J=8.8 Hz).

IR (KBr) 3528, 3400-2300, 1716, 1661, 1564, 1481 cm⁻¹.

Elemental Analysis (C₃₀H₃₄N₃O₈SCl.H₂O) Cal'd: C, 55.42, H 5.58; N, 6.46. Found: C, 55.05; H, 5.47; N, 6.16.

### EXAMPLE 145

2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methyl-1,3-thiazole-5-carboxylic acid

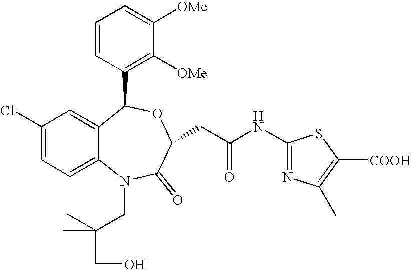

(1) Tert-butyl acetoacetate (10 g, 63.74 mmol) was dissolved in acetonitrile under the argon atmosphere, and copper (I) bromide (18.5 g, 82.86 mmol) and [hydroxy(tosyloxy)iodo]benzene (25 g, 63.74 mmol) were added under ice-cooling. The mixture was stirred at the same temperature for 30 minutes, water (200 ml) was added, and the mixture was further stirred for 30 minutes. The mixture was extracted with dichloromethane, and the organic layer was washed with an aqueous saturated sodium chloride solution. The organic layer was dried with anhydrous magnesium sulfate, concentrated under reduced pressure, and the resulting residue was purified by silica gel column chromatography (hexane:ethyl acetate=20:1) to obtain a yellow oil (4.88 g). A part of the resulting oil (1.66 g, 7.00 mmol) was dissolved in ethanol (15 ml), thiourea (0.53 g, 7.00 mmol) and sodium bicarbonate (1.18 g, 14.00 mmol) were added, and the mixture was stirred under heating at reflux for 1.5 hours. After allowing to cool, water and ethyl acetate were added, the layers were separated, and the organic layer was washed with an aqueous saturated sodium chloride solution. The organic layer was dried with anhydrous sodium sulfate, concentrated under reduced pressure, the resulting residue was purified by silica gel column chromatography (hexane:ethyl acetate=1:l), and the crude crystals were washed with hexane: diethyl ether (4:1) to obtain tert-butyl 2-amino-4-methyl-1,3-thiazole-5-carboxylate (0.64 g, yield 14% (2 steps)) as a pale yellow crystal.

m.p. 167.0-170.0° C.

¹H-NMR (200 MHz, CDCl₃) δ: 1.53 (9H, s), 2.49 (3H, s).

IR (KBr) 3600-2600, 1682, 1507 cm⁻¹.

(2) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.29 ml, 2.21 mmol) were added under ice-cooling, and the mixture was stirred at same temperature for 30 minutes. A solution of tert-butyl 2-amino-4-methyl-1,3-thiazole-5-carboxylate (0.41 g, 1.92 mmol) obtained in Example 145-(1) and pyridine (0.25 ml, 3.08 mmol) in N,N-dimethylformamide (3 ml) was added dropwise. The mixture was stirred at the same temperature for 1 hour, and stirred at room temperature for 3 hours. Water was added to the reaction solution, and the mixture was extracted with ethyl acetate. The organic layer was washed with 1N hydrochloric acid, water and an aqueous saturated sodium chloride solution. The organic layer was dried with anhydrous sodium sulfate, concentrated under reduced pressure, the resulting residue was purified by silica gel column chromatography (hexane:ethyl acetate=3:2), and the resulting crude crystals were washed with hexane:ethyl acetate (6:1). Drying under reduced pressure afforded tert-butyl 2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methyl-1,3-thiazole-5-carboxylate (0.5 g, yield 36.3%) as white crystals.

m.p. 211.4-213.0° C.

[α]_{D}²²=−71.4° (c=0.10, methanol).

¹H-NMR (300 MHz, CDCl₃) δ: 0.95 (3H, s), 1.02 (3H, s), 1.54 (9H, m), 2.02 (3H, s), 2.61 (3H, s), 2.95 (1H, dd, J=14.7, 6.0 Hz), 3.09 (1H, dd, J=14.7, 6.0 Hz), 3.54 (1H, d, J=14.1 Hz), 3.62 (3H, s), 3.73 (1H, d, J=11.1 Hz), 3.86 (1H, d, J=11.1 Hz), 3.89 (3H, s), 4.43 (1H, t, J=6.0 Hz), 4.59 (1H, d, J=14.1 Hz), 6.30 (1H, s), 6.66 (1H, d, J=2.1 Hz), 6.95-7.02 (1H, m), 7.13-7.21 (2H, m), 7.32 (1H, dd, J=8.7, 2.1 Hz), 9.63 (1H, brs).

IR (KBr) 2973, 1736, 1682, 1481, 1283 cm⁻¹.

Elemental Analysis (C₃₅H₄₂N₃O₉SCl) Cal'd: C, 58.69; H, 5.91; N, 5.87. Found: C, 58.44; H, 5.76; N, 5.74.

(3) Trifluoroacetic acid (4 ml) was added dropwise to tert-butyl 2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methyl-1,3-thiazole-5-carboxylate (0.4 g, 0.56 mmol) obtained in Example 145-(2) under ice-cooling. The mixture was stirred for 1.5 hours under ice-cooling, a temperature was raised to room temperature, and the mixture was stirred at room temperature for 1 hour. The mixture was concentrated under reduced pressure, ethyl acetate and water were added, the layers were separated, and the organic layer was washed with an aqueous saturated sodium chloride solution. The organic layer was dried with anhydrous sodium sulfate, and concentrated under reduced pressure. The resulting crude crystals were recrystallized from ethyl acetate-hexane to obtain 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methyl-1,3-thiazole-5-carboxylic acid (337 mg, yield 91.4%) as white crystals.

m.p. 195.0-197.0° C.

[α]_{D}²²=−87.3° (c=0.15, methanol).

¹H-NMR (200 MHz, DMSO-d₆) δ: 0.95 (3H, s), 1.03 (3H, s), 2.03 (3H, s), 2.65 (3H, s), 2.97-3.15 (1H, m), 3.32 (1H, dd, J=16.4, 7.8 Hz), 3.53-3.65 (4H, m), 3.74 (1H, d, J=11.0 Hz), 3.81-3.91 (4H, m), 4.51-4.60 (2H, m), 6.30 (1H, s), 6.66 (1H, s), 6.95-7.02 (1H, m), 7.14-7.18 (2H, m), 7.37 (2H, s).

IR (KBr) 3300-2200, 1738, 1682, 1481, 1283 cm⁻¹.

Elemental Analysis (C₃₁H₃₄N₃O₉SCl) Cal'd: C, 56.40; H, 5.19; N, 6.37. Found: C, 56.52; H, 5.38; N, 6.38.

(4) 2-[[[(3R,5S)-1-(3-Acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methyl-1,3-thiazole-5-carboxylic acid (337 mg, 0.51 mmol) obtained in Example 145-(3) was dissolved in methanol (10 ml), and potassium carbonate (212 mg, 1.531 mmol) was added. After the mixture was stirred at room temperature for 5 hours, and 1N hydrochloric acid was added to adjust the mixture to acidic. After the mixture was stirred at room temperature for 2 hours, the crystals were filtered off, and washed with a 50% aqueous methanol solution. Drying under reduced pressure afforded 2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methyl-1,3-thiazole-5-carboxylic acid (306 mg, yield 97.0%) as white crystals.

m.p. 251.0-252.0° C.

[α]_{D}²²=−90.1° (c=0.13, methanol).

¹H-NMR (300 MHz, DMSO-d₆) δ: 0.75 (3H, s), 0.85 (3H, s), 2.52 (3H, s), 2.94-2.99 (2H, m), 3.07 (1H, d, J=10.8 Hz), 3.19 (1H, d, J=10.8 Hz), 3.51 (3H, m), 3.69 (1H, d, J=13.8 Hz), 3.84 (3H, s), 4.31 (1H, d, J=13.8 Hz), 4.37 (1H, t, J=6.6 Hz), 4.55 (1H, brs), 6.10 (1H, s), 6.39 (1H, d, J=2.7 Hz), 7.75 (1H, d, J=9.0 Hz).

IR (KBr) 3443, 3400-2300, 1703, 1651, 1483, 1279 cm¹.

Elemental Analysis (C₂₉H₃₂N₃O₈SCl.0.5H₂O) Cal'd: C, 55.54; H, 5.30; N, 6.70. Found: C, 55.34; H, 5.39; N, 6.48.

### EXAMPLE 146

3-[2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-phenyl-1,3-thiazol-5-yl]propionic Acid

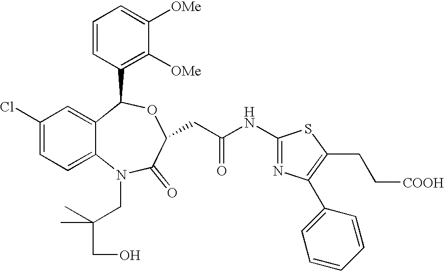

(1) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.29 ml, 2.21 mmol) were added under ice-cooling, the mixture was stirred at the same temperature for 30 minutes. Methyl 2-amino-4-phenyl-1,3-thiazole-5-propionate (0.5 g, 1.92 mmol) was added, and pyridine (0.25, 3.08 mmol) was added dropwise. The mixture was stirred at the same temperature, water was added to the reaction solution, and the mixture was extracted with ethyl acetate. The organic layer was washed with 1N hydrochloric acid, water and an aqueous saturated sodium chloride solution. The organic layer was washed with unhydrous anhydrous sodium sulfate, concentrated under reduced pressure, and the resulting residue was purified by silica gel column chromatography (hexane:ethyl acetate=1:2) to obtain methyl 3-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-phenyl-1,3-thiazol-5-yl]propionate (0.34 g, yield 52.9%) as a yellow amorphous powder.

m.p. 167.5-168.5° C.

[α]_{D}²²=−103.3° (c=0.16, methanol).

¹H-NMR (200 MHz, CDCl₃) δ: 0.94 (3H, s), 1.01 (3H, s), 2.02 (3H, s), 2.55-2.70 (3H, m), 2.94 (1H, dd, J=15.0, 7.4 Hz), 3.21 (2H, t, J=7.2 Hz), 3.53 (1H, d, J=13.8 Hz), 3.62 (3H, s), 3.66 (3H, s), 3.71 (1H, d, J=11.0 Hz), 3.85 (1H, d, J=11.0 Hz), 3.89 (3H, s), 4.38 (1H, dd, J=7.4, 5.8 Hz), 4.56 (1H, d, J=13.8 Hz), 6.25 (1H, s), 6.65 (1H, d, J=1.4 Hz), 6.90-7.03 (1H, m), 7.18 (1H, d, J=1.2 Hz), 7.20 (1H, s), 7.27-7.50 (5H, m), 7.56 (2H, dd, J=8.6, 1.4 Hz), 9.99 (1H, brs).

IR (KBr) 3179, 2953, 1738, 1682, 1557, 1481, 1279 cm⁻¹.

Elemental Analysis (C₃₉H₄₂N₃O₉SCl) Cal'd: C, 61.29; H, 5.54; N, 5.50. Found: C, 61.07; H, 5.45; N, 5.73.

(2) Methyl 3-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-phenyl-1,3-thiazol-5-yl]propionate (0.7 g, 0.92 mmol) obtained in Example 146-(1) was dissolved in ethanol (20 ml) and tetrahydrofuran (10 ml), a 2N aqueous sodium hydroxide solution (1.37 ml) was added at room temperature, and the mixture was stirred at room temperature for 4 hours and at 50° C. for 6 hours. After allowing to cool, 1N hydrochloric acid was added to adjust the mixture to acidic, the mixture was concentrated under reduced pressure, and the residue was extracted with ethyl acetate. The organic layer was washed with water and aqueous saturated sodium chloride solution. The organic layer was dried with anhydrous sodium sulfate, and concentrated under reduced pressure. The resulting residue was purified by silica gel column chromatography (hexane:ethyl acetate=1:2) to obtain 3-[2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-phenyl-1,3-thiazol-5-yl]propionic acid (0.34 g, yield 52.9%) as a pale foam.

[α]_{D}²²=−102.5° (c=0.14, methanol).

¹H-NMR (200 MHz, CDCl₃) δ: 0.64 (3H, s), 1.03 (3H, s), 2.02 (3H, s), 2.57-2.70 (2H, m), 2.85 (1H, dd, J=15.8, 5.2 Hz), 3.09-3.29 (3H, m), 3.38 (1H, d, J=14.4 Hz), 3.58-3.70 (5H, m), 3.89 (3H, s), 4.40-4.59 (2H, m), 6.19 (1H, s), 6.62 (1H, s), 6.92-7.08 (1H, m), 7.10-7.21 (2H, m), 7.26-7.50 (7H, m).

IR (KBr) 3700-2300, 1661, 1559, 1481, 1281 cm⁻¹.

Elemental Analysis (C₃₆H₃₈N₃O₈SCl.0.5O₂O) Cal'd: C, 60.29; H, 5.48; N, 5.86. Found: C, 60.51; H, 5.77; N, 5.76.

### EXAMPLE 147

4-[2-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-(4-chlorophenyl)-1,3-thiazol-5-yl]butanoic acid

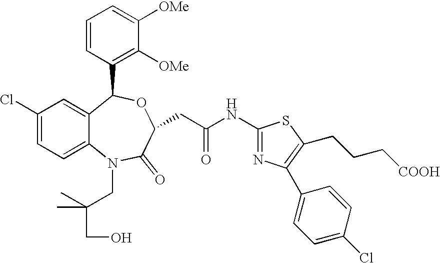

(1) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.29 ml, 2.21 mmol) were added under ice-cooling, the mixture was stirred at the same temperature for 30 minutes. Ethyl 4-[2-amino-4-(4-chlorophenyl)-1,3-thiazol-5-yl]butanoate (0.78 g, 1.92 mmol) was added, and pyridine (0.25, 3.08 mmol) was added dropwise. The mixture was stirred at the same temperature, water was added to the reaction solution, and the mixture was extracted with ethyl acetate. The organic layer was washed with 1N hydrochloric acid, water and an aqueous saturated sodium chloride solution. The organic layer was washed with anhydrous sodium sulfate, concentrated under reduced pressure, and the resulting residue was purified by silica gel column chromatography (hexane:ethyl acetate=2:1) to obtain ethyl 4-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-(4-chlorophenyl)-1,3-thiazol-5-yl]butanoate (0.48 g, yield 30.2%) as a colorless foam.

[α]_{D}²²=−111.7° (c=0.15, methanol).

¹H-NMR (200 MHz, CDCl₃) δ: 0.95 (3H, s), 1.02 (3H, s), 1.21 (3H, t, J=7.4 Hz), 1.98 (2H, t, J=7.0 Hz), 2.02 (3H, s), 2.33 (2H, t, J=7.0 Hz), 2.70-3.06 (4H, m), 3.54 (1H, d, J=14.4 Hz), 3.62 (3H, s), 3.72 (1H, d, J=11.4 Hz), 3.86 (1H, d, J=11.4 Hz), 3.90 (3H, s), 4.08 (2H, q, J=7.4 Hz), 4.36-4.45 (1H, m), 4.57 (1H, d, J=14.4 Hz), 6.29 (1H, s), 6.66 (1H, d, J=1.8 Hz), 6.95-7.02 (1H, m), 7.12-7.24 (2H, m), 7.30-7.41 (4H, m), 7.51 (2H, d, J=8.4 Hz), 9.68 (1H, brs).

IR (KBr) 2973, 1732, 1680, 1553, 1481, 1281, 1248 cm⁻¹.

Elemental Analysis (C₄₁H₄₅N₃O₉SCl₂) Cal'd: C, 59.56; H, 5.49; N, 5.08. Found: C, 59.33; H, 5.46; N, 5.25.

(2) Ethyl 4-[2-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-(4-chlorophenyl)-1,3-thiazol-5-yl]butanoate (0.4 g, 0.48 mmol) obtained in Example 147-(1) was dissolved in ethanol (9 ml), and a 2N aqueous sodium hydroxide solution (0.73 ml) was added at room temperature. The mixture was stirred at room temperature for 22 hours, and stirred at 50° C. for 7 hours. 1N hydrochloric acid was added to adjust the mixture to acidic, water was added, and the mixture was stirred for 1 hour. The crystals were filtered off, and dried under reduced pressure to obtain 4-[2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-(4-chlorophenyl)-1,3-thiazol-5-yl]butanoic acid (0.3 g, yield 54.6%) as white crystals.

[α]_{D}²²=−97.9° (c=0.06, methanol).

¹H-NMR (200 MHz, DMSO-d₆) δ: 0.76 (3H, s), 0.86 (3H, s), 1.75-1.90 (2H, m), 2.29 (2H, t, J=6.8 Hz), 2.80-3.00 (4H, m), 3.01-3.21 (2H, m), 3.52 (3H, s), 3.69 (1H, d, J=14.0 Hz), 3.84 (3H, s), 4.25-4.42 (2H, m), 4.56 (1H, brs), 6.10 (1H, s), 6.40 (1H, d, J=2.4 Hz), 7.00-7.23 (3H, m), 7.45-7.70 (5H, m), 7.76 (1H, d, J=9.2 Hz).

IR (KBr) 3700-2300, 1659, 1553, 1481, 1281 cm⁻¹.

Elemental Analysis (C₃₇H₃₉N₃O₈Cl₂.H₂O) Cal'd: C, 57.36; H, 5.33; N, 5.42. Found: C, 57.32; H, 5.35; N, 5.17.

### EXAMPLE 148

2-[[5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1H-benzimidazol-2-yl]sulfanyl]acetic acid

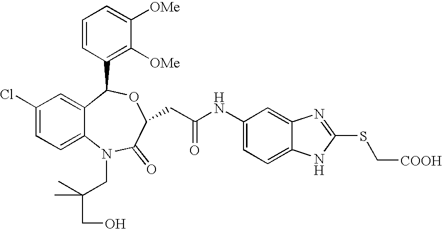

(1) 2-Mercaptobenzimidazole (5 g, 25.62 mol) was dissolved in N,N-dimethylformamide (85 ml), and potassium carbonate (3.65 g, 26.38 mol) and ethyl bromoacetate (2.9 ml, 26.13 mol) were added. The mixture was stirred at room temperature for 30 minutes. The mixture was neutralized with the addition of 6N hydrochloric acid under ice-cooling, water and ethyl acetate were added, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=2:1), and dried under reduced pressure to obtain ethyl 2-[(5-nitro-1H-benzimidazol-2-yl)sulfanyl)acetate (4.21 g, yield 58.4%) as white crystals.

m.p. 113.5-114.0° C.

¹H-NMR (200 MHz, CDCl₃): 1.37 (3H, t, J=7.0 Hz), 4.07 (2H, s), 4.34 (2H, q, J=7.0 Hz), 7.46 (1H, d, J=9.2 Hz), 8.08 (1H, dd, J=9.2, 2.2 Hz), 8.34 (1H, brs).

IR (KBr) 3460-3200, 1732, 1520, 1339 cm⁻¹.

Elemental Analysis (C₁₁H₁₁N₃O₄S) Cal'd: C, 46.97; H, 3.94; N, 14.94. Found: C, 47.04; H, 3.64; N, 14.65.

(2) Ethyl 2-[(5-nitro-1H-benzimidazol-2-yl)sulfanyl)acetate (1.5 g, 5.33 mol) obtained in Example 148-(1) was dissolved in acetic acid (5 ml), and zinc (4.17 g, 63.79 mol) was added. The mixture was stirred at 50° C. for 2 hours. The reaction solution was concentrated, the resulting residue was diluted with ethyl acetate, and washed with an aqueous saturated sodium bicarbonate solution, water and an aqueous saturated sodium chloride solution. This was dried with anhydrous sodium sulfate, and concentrated under reduced pressure. The resulting residue was purified by silica gel column chromatography (ethyl acetate). Ethyl acetate was added to the resulting crystals (0.94 g), 4N hydrogen chloride-ethyl acetate (0.93 ml) was added, the mixture was stirred at room temperature for 30 minutes, and the crystals were filtered and washed with ethyl acetate. Drying under reduced pressure afforded ethyl 2-[(5-amino-1H-benzimidazol-2-yl)sulfanyl]acetate hydrochloride (yield 50.1%) as a grayish-white crystal.

m.p. 114.1-114.2° C.

¹H-NMR (200 MHz, DMSO-d₆) δ: 1.17 (3H, t, J=7.0 Hz), 4.13 (2H, q, J=7.0 Hz), 4.26 (2H, s), 7.14 (1H, dd, J=8.4, 1.8 Hz), 7.48 (1H, d, J=1.8 Hz), 7.54 (1H, d, J=8.4 Hz).

IR (KBr) 3400-2500, 1726, 1404 cm⁻¹.

Elemental Analysis (C₁₁H₁₄N₃O₂SCl.H₂O) Cal'd: C, 45.07; H, 5.02; N, 14.33. Found: C, 45.01; H, 4.92; N, 14.21.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepin-3-acetic acid (1.0 g, 1.92 mmol) obtained in Example 1-(1) was dissolved in tetrahydrofuran (10 ml), and one droplet of N,N-dimethylformamide was added. Thionyl chloride (0.21 ml, 2.89 mmol) was added at room temperature, the mixture was stirred for 1.5 hours, concentrated under reduced pressure, and dissolved in tetrahydrofuran (5 ml). Ethyl 2-[(5-amino-1H-benzimidazol-2-yl)sulfanyl]acetate hydrochloride (0.55 g, 1.92 mmol) obtained in Example 148-(2) was dissolved in tetrahydrofuran (10 ml), and triethylamine (0.67 ml, 4.81 mmol) was added. The previously prepared acid chloride solution was added dropwise at room temperature, and the mixture was stirred at the same temperature for 2 hours. Water and ethyl acetate were added to the reaction solution, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=2:5), and dried under reduced pressure to obtain ethyl 2-[[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1H-benzimidazol-2-yl]sulfanyl]acetate (964 mg, yield 66.5%) as a colorless foam.

[α]_{D}²²=−86.0° (c=0.49, methanol).

¹H-NMR (200 MHz, CDCl₃) δ: 0.95 (3H, s), 1.00 (3H, s), 1.31 (3H, t, J=7.0 Hz), 1.98 (3H, s), 2.86 (1H, dd, J=14.4, 5.8 Hz), 3.13 (1H, dd, J=14.4, 7.8 Hz), 3.52 (1H, d, J=14.4 Hz), 3.61 (3H, s), 3.74 (1H, d, J=11.4 Hz), 3.83-3.96 (2H, m), 3.88 (3H, s), 4.09 (1H, d, J=16.2 Hz), 4.27 (2H, q, J=7.0 Hz), 4.50-4.56 (1H, m), 4.59 (1H, d, J=14.4 Hz), 6.31 (1H, s), 6.64 (1H, s), 6.80 (1H, d, J=8.4 Hz), 6.97 (1H, dd, J=7.6, 1.8 Hz), 7.08-7.45 (6H, m), 7.94 (1H, s), 8.42 (1H, s), 10.64 (1H, s).

IR (KBr) 3400-3100, 1736, 1661, 1481 cm⁻¹.

Elemental Analysis (C₃₇H₄₁N₄O₉ClS.H₂O) Cal'd: C, 57.62; H, 5.62; N, 7.26. Found: C, 57.90; H, 5.62; N, 6.98.

(4) Ethyl 2-[[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1H-benzimidazol-2-yl]sulfanyl]acetate (0.5 g, 0.66 mmol) obtained in Example 148-(3) was dissolved in tetrahydrofuran (5 ml) and ethanol (1.5 ml), a 2N aqueous sodium hydroxide solution (1.33 ml) was added at room temperature, and the mixture was stirred at the same temperature for 1.5 hours. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sodium sulfate, and concentrated under reduced pressure. The resulting residue was recrystallized from tetrahydrofuran-ethyl acetate, and dried under reduced pressure to obtain 2-[[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1H-benzimidazol-2-yl]sulfanyl]acetic acid (356 mg, yield 78.5%) as white crystals.

m.p. 187.9-188.9° C.

[α]_{D}²²=−89.2° (c=0.44, methanol).

¹H-NMR (200 MHz, DMSO-d₆) δ: 0.77 (3H, s), 0.86 (3H, s), 2.83 (2H, d, J=6.6 Hz), 3.00-3.25 (2H, m), 3.52 (3H, s), 3.68 (1H, d, J=14.2 Hz), 3.84 (3H, s), 4.12 (2H, s), 4.27-4.40 (2H, m), 4.56 (1H, brs), 6.11 (1H, s), 6.40 (1H, d, J=2.6 Hz), 7.05-7.20 (4H, m), 7.36 (1H, d, J=8.8 Hz), 7.56 (1H, dd, J=8.8, 2.6 Hz), 7.74 (1H, d, J=8.8 Hz), 7.85 (1H, s), 10.04 (1H, s).

IR (KBr) 3700-2200, 1659, 1595, 1481 cm⁻¹.

Elemental Analysis (C₃₃H₃₅N₄O₈ClS.1.2H₂O) Cal'd: C, 56.24; H, 5.35; N, 7.95. Found: C, 56.23; H, 5.51; N, 8.05.

### EXAMPLE 149

5-[[[(3R,5S)-5-(2,3-dimethoxyphenyl)-7-chloro-1-(3-hydroxy-2,2-dimethylpropyl)-2-oxo-1,2,3,5-tetrahydro-4,1-benzoxazepin-3-yl]acetyl]amino]-7-methoxy-1-benzofran-2-carboxylic acid

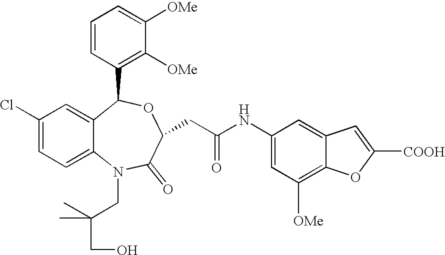

(1) 2-Hydroxy-5-nitro-m-anisaldehyde (7.0 g, 0.03 mol) was dissolved in N,N-dimethylformamide (140 ml), and ethyl bromoacetate (5.9 ml, 0.05 mol) was added. Potassium carbonate (12.3 g, 0.09 mol) was added at room temperature, and the mixture was stirred at 70° C. for 15 hours. Potassium carbonate (4.9 g, 0.04 mol) and ethyl bromoacetate (1.98 ml, 0.02 mol) were added, and the mixture was further stirred at 70° C. for 20 hours. After allowing to cool, the mixture was neutralized using 1N hydrochloric acid, and the layers were separated. Ethyl acetate was added to the aqueous layer, the mixture was extracted, the organic layers were combined, and washed with water and an aqueous saturated sodium chloride solution. The organic layer was dried with anhydrous sodium sulfate, and concentrated under reduced pressure. Methanol (400 ml) was added to the resulting crystals, heated to dissolve them, allowed to cool, and the crystals were filtered off. The crystals were dried under reduced pressure to obtain ethyl 7-methoxy-5-nitro-1-benzofuran-2-carboxylate (3.72 g, yield 39.5%) as white crystals.

m.p. 164.8-164.9° C.

¹H-NMR (200 MHz, CDCl₃): 1.44 (3H, t, J=7.4 Hz), 4.11 (3H, s), 4.77 (2H, q, J=7.4 Hz), 7.63 (1H, s), 7.82 (1H, d, J=1.8 Hz), 8.26 (1H, d, J=1.8 Hz).

IR (KBr) 1718, 1537, 1350, 1327 cm⁻¹.

Elemental Analysis (C₁₂H₁₁NO₅) Cal'd: C, 54.34; H, 4.18; N, 5.28. Found: C, 54.40; H, 4.23; N, 5.06.

(2) Ethyl 7-methoxy-5-nitro-1-benzofuran-2-carboxylate (3.0 g, 0.01 mol) obtained in Example 149-(1) was suspended in ethyl acetate (90 ml), and nitrogen replacement was performed. 10% palladium carbon (0.6 g) was placed therein, and hydrogen was introduced. The mixture was stirred at room temperature for 5 hours, the catalyst was filtered, and the filtrate was concentrated under reduced pressure. Ethyl acetate (30 ml) was added to the residue, 4N hydrogen chloride-ethyl acetate (2.83 ml) was added, the mixture was stirred at room temperature for 1 hour, and the crystals were washed with ethyl acetate. The crystals were dried under reduced pressure (50° C.) to obtain ethyl 5-amino-7-methoxy-1-benzofuran-2-carboxylate hydrochloride (2.77 g, yield 90.1%) as white crystals.

m.p. 239.0-239.2° C.

¹H-NMR (200 MHz, CDCl₃): 1.41 (3H, t, J=7.0 Hz), 4.07 (3H, s), 4.42 (2H, q, J=7.0 Hz), 7.00 (1H, d, J=1.8 Hz), 7.33 (1H, d, J=1.8 Hz), 7.65 (1H, s).

IR (KBr) 3312, 2838, 2589, 1715, 1597, 1586, 1312 cm⁻¹.

Elemental Analysis (C₁₂H₁₄NO₄Cl) Cal'd: C, 53.05; H, 5.19; N, 5.16. Found: C, 52.81; H, 5.25; N, 5.08.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepin-3-acetic acid obtained in Example 1-(1) (1.0 g, 1.92 mmol) was dissolved in tetrahydrofuran (10 ml), and one droplet of N,N-dimethylformamide was added. Thionyl chloride (0.21 ml, 2.89 mmol) was added under ice-cooling, a temperature was raised to room temperature, the mixture was stirred for 1 hour, concentrated under reduced pressure, and dissolved in tetrahydrofuran (5 ml). Ethyl 5-amino-7-methoxy-1-benzofuran-2-carboxylate hydrochloride (0.52 g, 1.92 mmol) was suspended in tetrahydrofuran (10 ml), and triethylamine (0.67 ml, 4.81 mmol) was added. The previously prepared acid chloride solution was added thereto at room temperature, and the mixture was stirred at the same temperature for 2 hours. Water and ethyl acetate were added to the reaction solution, the layers were separated, and the organic layer was washed with water, an aqueous saturated sodium chloride solution. The organic layer was dried with anhydrous sodium sulfate, and concentrated under reduced pressure. The resulting residue was purified by silica gel column chromatography (hexane:ethyl acetate=2:1) to obtain ethyl 5-[[[(3R,5S)-1-(3-acetoxy-2,2-dimethylpropyl)-5-(2,3-dimethoxyphenyl)-7-chloro-2-oxo-1,2,3,5-tetrahydro-4,1-benzoxazepin-3-yl]acetyl]amino]-7-methoxy-1-benzofuran-2-carboxylate (0.86 g, yield 60.7%) as a pale brown foam.

[α]_{D}²²=−95.7° (c=0.40, methanol).

¹H-NMR (200 MHz, CDCl₃): 0.95 (3H, s), 1.00 (3H, s), 1.41 (3H, t, J=7.0 Hz), 1.98 (3H, s), 2.88 (1H, dd, J=14.8, 6.0 Hz), 3.07 (1H, dd, J=14.8, 7.6 Hz), 3.54 (1H, d, J=14.4 Hz), 3.60 (3H, s), 3.74 (1H, d, J=14.4 Hz), 3.80 (1H, d, J=13.2 Hz), 3.88 (3H, s), 3.92 (3H, s), 4.42 (2H, q, J=7.0 Hz), 4.40-4.60 (2H, m), 6.30 (1H, s), 6.64 (1H, s), 6.97 (1H, d, J=8.0 Hz), 7.00-7.22 (3H, m), 7.33 (2H, s), 7.39 (1H, s), 7.43 (1H, d, J=1.4 Hz), 8.55 (1H, brs).

IR (KBr) 3337, 2965, 1717, 1651, 1559 cm⁻¹.

Elemental Analysis (C₃₈H₄₁N₂O₁₁Cl.0.5H₂O) Cal'd: C, 61.16; H, 5.67; N, 3.75. Found: C, 61.22; H, 5.64; N, 3.36.

(4) Ethyl 5-[[[(3R,5S)-1-(3-acetoxy-2,2-dimethylpropyl)-5-(2,3-dimethoxyphenyl)-7-chloro-2-oxo-1,2,3,5-tetrahydro-4,1-benzoxazepin-3-yl]acetyl]amino]-7-methoxy-1-benzofuran-2-carboxylate (0.75 g, 1.02 mmol) obtained in Example 149-(3) was dissolved in tetrahydrofuran (3 ml) and ethanol (1 ml), a 2N aqueous sodium hydroxide solution (1 ml) was added, and the mixture was stirred at the same temperature. The mixture was neutralized using 1N hydrochloric acid, and extracted with ethyl acetate. The organic layer was washed with an aqueous saturated sodium chloride solution, dried with anhydrous sodium sulfate and concentrated under reduced pressure. The resulting residue was purified by silica gel column chromatography (ethyl acetate) to obtain 5-[[[(3R,5S)-5-(2,3-dimethoxyphenyl)-7-chloro-1-(3-hydroxy-2,2-dimethylpropyl)-2-oxo-1,2,3,5-tetrahydro-4,1-benzoxazepin-3-yl]acetyl]amino]-7-methoxy-1-benzofran-2-carboxylic acid (0.2 g, yield 28.7%) as white crystals.

m.p. 175.4-175.5° C.

[α]_{D}²²=−117.7° (c=0.40, methanol).

¹H-NMR (200 MHz, CDCl₃) δ: 0.67 (3H, s), 1.06 (3H, s), 2.89 (1H, dd, J=14.4, 5.8 Hz), 3.06 (1H, dd, J=14.4, 7.6 Hz), 3.21 (1H, d, J=12.2 Hz), 3.41 (1H, d, J=13.8 Hz), 3.60-3.68 (4H, m), 3.89 (3H, s), 3.97 (3H, s), 4.43-4.55 (2H, m), 6.21 (1H, s), 6.63 (1H, d, J=1.6 Hz), 6.99 (1H, dd, J=7.4, 2.6 Hz), 7.10-7.19 (3H, m), 7.36 (2H, s), 7.39 (1H, dd, J=8.8, 1.6 Hz), 7.49 (1H, s), 8.08 (1H, brs).

IR (KBr) 3600-2400, 1717, 1653, 1481 cm⁻¹.

Elemental Analysis (C₃₄H₃₅N₂O₁₀Cl.0.5H₂O) Cal'd: C, 60.40; H, 5.37; N, 4.14. Found: C, 60.33; H, 5.38; N, 3.92.

### EXAMPLE 150

5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-ethoxy-1-benzofuran-2-carboxylic acid

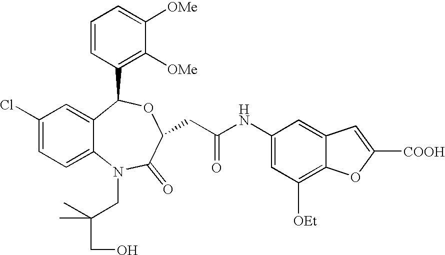

(1) Ethyl 7-methoxy-5-nitro-1-benzofuran-2-carboxylate (16 g, 60.33 mmol) was suspended in acetic acid (80 ml), and 48% hydrobromic acid (160 ml) was added. The mixture was stirred under heating at reflux for 4 days. After allowing to stand, water (100 ml) was added, the mixture was stirred for 1 hour, the crystals were filtered off, and washed with water. Drying under reduced pressure (50° C.) afforded 7-hydroxy-5-nitro-1-benzofuran-2-carboxylic acid (9.55 g, yield 70.9%) as a brown crystal.

m.p. 293.5-294.4° C.

¹H-NMR (200 MHz, DMSO-d₆) δ: 7.71 (1H, d, J=2.6 Hz), 7.81 (1H, s), 8.21 (1H, d, J=2.6 Hz), 11.4 (1H, brs).

IR (KBr) 3648, 3400-2200, 1699, 1524 cm⁻¹.

Elemental Analysis (C₉H₅NO₆.0.5H₂O) Cal'd: C, 46.56; H, 2.61; N, 6.03. Found: C, 46.72; H, 2.76; N, 5.84.

(2) 7-hydroxy-5-nitro-1-benzofuran-2-carboxylic acid (7.55 g, 33.84 mmol) obtained in Example 150-(1) was suspended in methanol (75.5 ml), and concentrated sulfuric acid (3.8 ml) was added. The mixture was stirred under heating at reflux for 36 hours. After allowing to cool, water (76 ml) was added, the mixture was stirred at room temperature for 1 hour, the crystals were filtered off, and washed with water. Drying under reduced pressure (50° C.) afforded methyl 7-hydroxy-5-nitro-1-benzofuran-2-carboxylate (7.30 g, yield 91.0%) as a brown crystal.

m.p. 251.5-252.7° C.

¹H-NMR (200 MHz, DMSO-d₆) δ: 3.93 (3H, s), 7.72 (1H, d, J=2.2 Hz), 7.91 (1H, s), 8.22 (1H, d, J=2.2 Hz).

IR (KBr) 3282, 1690, 1584, 1582, 1331 cm⁻¹.

Elemental Analysis (C₁₀H₇NO₆) Cal'd: C, 50.64; H, 2.97; N, 5.91. Found: C, 50.38; H, 2.95; N, 5.82.

(3) Methyl 7-hydroxy-5-nitro-1-benzofuran-2-carboxylate (1.0 g, 4.22 mmol) obtained in Example 150-(2) was dissolved in N,N-dimethlformamide (20 ml), and potassium carbonate (0.76 g, 5.48 mmol) and iodomethane (0.4 ml, 5.06 mmol) were added at room temperature. After stirred at the same temperature for 14 hours, water, ethyl acetate and tetrahydrofuran were added to the reaction solution, and the layers were separated. The organic layer was washed with 1N hydrochloric acid, water and an aqueous saturated sodium chloride solution, dried with anhydrous sodium sulfate, and concentrated under reduced pressure. The resulting crude crystals were suspended in ethyl acetate (10 ml)-hexane (10 ml), and the suspension was stirred at room temperature for 1 hour. The crystals were filtered off, washed with hexane, and dried under reduced pressure (50° C.) to obtain methyl 7-hydroxy-5-nitro-1-benzofuran-2-carboxylate (1.06 g, yield 94.8%) as pale a brown crystal.

m.p. 223.8-224.0° C.

¹H-NMR (200 MHz, CDCl₃): 1.58 (3H, t, J=7.4 Hz), 4.00 (3H, s), 4.36 (2H, q, J=7.4 Hz), 7.63 (1H, s), 7.81 (1H, d, J=1.8 Hz), 8.24 (1H, d, J=1.8 Hz).

IR (KBr) 1746, 1526, 1346, 1319 cm⁻¹.

Elemental Analysis (Cl₂H₁₁NO₆) Cal'd: C, 54.34; H, 4.18; N, 5.28. Found: C, 54.13; H, 4.31; N, 4.99.

(4) Methyl 7-hydroxy-5-nitro-1-benzofuran-2-carboxylate (0.80 g, 3.02 mmol) obtained in Example 150-(3) was dissolved in tetrahydrofuran (16 ml), and nitrogen replacement was performed. 10% palladium carbon (160 mg) was placed therein, and hydrogen was introduced. After stirred at room temperature for 5 hours, the catalyst was filtered off, and the filtrate was concentrated under reduced pressure. Ethyl acetate was added to the resulting residue, 4N hydrochloric aid/ethyl acetate (0.75 ml) was added, the mixture was stirred at room temperature for 1 hour, the crystals were filtered off, and washed with ethyl acetate. Drying under reduced pressure (50° C.) afforded methyl 5-amino-7-ethoxy-1-benzofuran-2-carboxylate hydrochloride (0.75 g, yield 91.5%) as white crystals.

m.p. 236.7-237.3° C.

¹H-NMR (200 MHz, DMSO-d₆) δ 1.46 (3H, t, J=7.0 Hz), 3.91 (3H, s), 4.24 (2H, q, J=7.0 Hz), 7.09 (1H, d, J=1.8 Hz), 7.35 (1H, d, J=1.8 Hz), 7.84 (1H, s).

IR (KBr) 3200-2200, 1728, 1587, 1338, 1308 cm¹.

Elemental Analysis (C₁₂H₁₄NO₄Cl) Cal'd: C, 53.05; H, 5.19; N, 5.16. Found: C, 52.85; H, 5.31; N, 5.00.

(5) (3R,5S)-1-(3-Acetoxy-2,2-dimethylpropyl)-7-chloro-5-(2,3-dimethoxyphenyl)-1,2,3,5-tetrahydrofuran-2-oxo-4,1-benzoxazepin-3-acetic acid (1.0 g, 1.92 mmol) was dissolved in N,N-dimethylformamide (5 ml) under the argon atmosphere. Triethylamine (0.21 ml, 1.96 mmol) and isobutyl chloroformate (0.28 ml, 2.22 mmol) were added under ice-cooling, and the mixture was stirred at the same temperature for 30 minutes. Methyl 5-amino-7-ethoxy-1-benzofuran-2-carboxylate hydrochloride (0.52 g, 1.92 mmol) obtained in Example 150-(4) was added, and pyridine (0.25 ml, 3.08 mmol) was added dropwise. After stirred at the same temperature, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=3:2), the resulting crystals were recrystallized from ethyl acetate (20 ml)-hexane (60 ml), and dried under reduced pressure (50° C.) to obtain methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-ethoxy-1-benzofuran-2-carboxylate (1.15 g, yield 81.1%) as white crystals.

m.p. 139.5-141.0° C.

[α]_{D}²²=−99.4° (c=0.27, methanol).

¹H-NMR (200 MHz, CDCl₃) δ: 0.96 (3H, s), 1.02 (3H, s), 1.50 (3H, t, J=7.4 Hz), 2.02 (3H, s), 2.84 (1H, dd, J=14.0, 5.8 Hz), 3.00 (1H, dd, J=14.0, 7.0 Hz), 3.54 (1H, d, J=14.2 Hz), 3.62 (3H, s), 3.74 (1H, d, J=11.0 Hz), 3.88 (1H, d, J=11.0 Hz), 3.90 (3H, s), 3.96 (3H, s), 4.24 (2H, q, J=7.4 Hz), 4.37-4.47 (1H, m), 4.57 (1H, d, J=14.2 Hz), 6.31 (1H, s), 6.65 (1H, d, J=2.2 Hz), 6.98 (1H, dd, J=7.4, 1.8 Hz), 7.05-7.21 (3H, m), 7.30-7.39 (2H, m), 7.43 (1H, d, J=1.8 Hz), 7.46 (1H, s), 7.92 (1H, s).

IR (KBr) 1736, 1678, 1665, 1481 cm⁻¹.

Elemental Analysis (C₃₇H₄₁N₂O₁₁Cl.0.5H₂O) Cal'd: C, 61.16; H, 5.67; N, 3.75. Found: C, 61.00; H, 5.60; N, 3.66.

(6) Methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-ethoxy-1-benzofuran-2-carboxylate (0.7 g, 0.95 mmol) obtained in Example 150-(5) was dissolved in tetrahydrofuran (7 ml) and ethanol (3 ml), a 2N aqueous sodium hydroxide solution (1.9 ml) was added at room temperature, and the mixture was stirred at 50° C. for 2 hours. Allowing to cool, the mixture was neutralized using 1N hydrochloric acid, concentrated under reduced pressure, ethyl acetate and water were added, and the layers were separated. The organic layer was washed with a 0.4N aqueous sodium hydroxide solution, water, 1N hydrochloric acid, water and an aqueous saturated sodium chloride solution, dried with anhydrous sodium sulfate, and concentrated under reduced pressure. The resulting crude crystals were recrystallized from ethyl acetate (35 ml)-hexane (17.5 ml), and dried under reduced pressure to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-ethoxy-1-benzofuran-2-carboxylic acid (0.35 g, yield 54.1%) as white crystals.

m.p. 181.0-181.5° C.

[α]_{D}²²=−111.1° (c=0.28, methanol).

¹H-NMR (200 MHz, DMSO-d₆) δ: 0.77 (3H, s), 0.86 (3H, s), 1.43 (3H, t, J=7.0 Hz), 2.84 (2H, d, J=7.0 Hz), 3.10-3.30 (2H, m), 3.53 (3H, s), 3.68 (1H, d, J=13.4 Hz), 3.83 (3H, s), 4.18 (2H, q, J=7.0 Hz), 4.27-4.40 (2H, m), 4.55 (1H, brs), 6.11 (1H, s), 6.40 (1H, d, J=2.6 Hz), 7.07-7.21 (4H, m), 7.51-7.65 (3H, m), 7.75 (1H, d, J=8.8 Hz), 10.1 (1H, s).

IR (KBr) 3600-2300, 1736, 1692, 1630, 1574, 1472, 1427 cm⁻¹.

Elemental Analysis (C₃₅H₃₇N₂O₁₀Cl.0.5H₂O) Cal'd: C, 60.91; H, 5.55; N, 4.06. Found: C, 60.70; H, 5.74; N, 3.81.

### EXAMPLE 151

5-[[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-propoxy-1-benzofuran-2-carboxylic acid

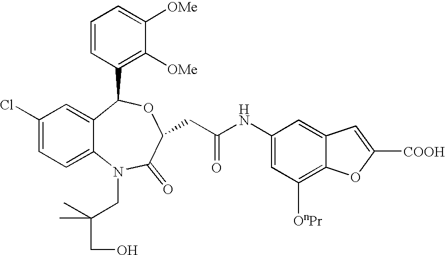

(1) Methyl 7-hydroxy-5-nitro-1-benzofuran-2-carboxylate (1.0 g, 4.22 mmol) was dissolved in N,N-dimethylformamide (20 ml), and potassium carbonate (0.76 g, 5.48 mmol) and iodo n-propane (0.49 ml, 5.06 mmol) were added at room temperature. After stirred at the same temperature for 14 hours, water (20 ml) was added to the reaction solution, and the mixture was stirred at room temperature for 3 hours. The crystals were filtered off, washed with methanol: water (4:1) and water, and dried under reduced pressure (50° C.) to obtain methyl 7-propoxy-5-nitro-1-benzofuran-2-carboxylate (1.11 g, yield 94.3%).

m.p. 157.0-157.2° C.

¹H-NMR (200 MHz, CDCl₃) δ: 1.12 (3H, t, J=7.4 Hz), 1.98 (2H, m), 4.00 (3H, s), 4.24 (2H, t, J=6.6 Hz), 7.63 (1H, s), 7.81 (1H, d, J=1.8 Hz), 8.24 (1H, d, J=1.8 Hz), 8.69 (1H, d, J=2.2 Hz).

IR (KBr) 1730, 1586, 1526, 1356, 1325 cm⁻¹.

Elemental Analysis (C₁₃H₁₃NO₆) Cal'd: C, 55.91; H, 4.69; N, 5.02. Found: C, 55.83; H, 4.68; N, 5.25.

(2) Methyl 7-propoxy-5-nitro-1-benzofuran-2-carboxylate (0.8 g, 2.87 mmol) obtained in Example 151-(1) was dissolved in tetrahydrofuran (16 ml), and nitrogen replacement was performed. 10% palladium carbon (160 mg) was placed therein, and hydrogen was introduced. After stirred at room temperature for 5 hours, the catalyst was filtered, and the filtrate was concentrated under reduced pressure. Ethyl acetate was added to the resulting residue, 4N hydrogen chloride-ethyl acetate (0.72 ml) was added, the mixture was stirred at room temperature for 1 hour, the crystals were filtered, and washed with ethyl acetate. Drying under reduced pressure (50° C.) afforded methyl 5-amino-7-propoxy-1-benzofuran-2-carboxylate hydrochloride (0.78 g, yield 95.3%) as white crystals.

m.p. 173.5-175.5° C.

¹H-NMR (200 MHz, DMSO-d₆) δ: 1.04 (3H, t, J=6.8 Hz), 1.86 (2H, m), 3.91 (3H, s), 4.15 (2H, q, J=6.8 Hz), 7.09 (1H, d, J=2.0 Hz), 7.33 (1H, d, J=2.0 Hz), 7.84 (1H, s).

IR (KBr) 3200-2350, 1736, 1725, 1588, 1337, 1308 cm⁻¹.

Elemental Analysis (C₁₃H₁₆NO₄Cl) Cal'd: C, 54.65; H, 5.64; N, 4.90. Found: C, 54.55; H, 5.79; N, 4.83.

(3) (3R,5S)-1-(3-Acetoxy-2,2-dimethylpropyl)-7-chloro-5-(2,3-dimethoxyphenyl)-1,2,3,5-tetrahydrofuran-2-oxo-4,1-benzoxazepin-3-acetic acid (1.0 g, 1.92 mmol) was dissolved in N,N-dimethylformamide (5 ml) under the argon atmosphere. Triethylamine (0.21 ml, 1.96 mmol) and isobutyl chloroformate (0.28 ml, 2.22 mmol) were added under ice-cooling, and the mixture was stirred at the same temperature for 30 minutes. Methyl 5-amino-7-propoxy-1-benzofuran-2-carboxylate hydrochloride (0.55 g, 1.92 mmol) obtained in Example 151-(2) was added, and pyridine (0.25 ml, 3.08 mmol) was added dropwise. After stirred at the same temperature for 2 hour, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting crystals were recrystallized from ethyl acetate (20 ml)-hexane (60 ml), and dried under reduced pressure (50° C.) to obtain methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-propoxy-1-benzofuran-2-carboxylate (1.29 g, yield 89.3%) as white crystals.

m.p. 146.1-147.1° C.

[α]_{D}²²=−99.3° (c=0.29, methanol).

¹H-NMR (200 MHz, CDCl₃) δ: 0.96 (3H, s), 1.02 (3H, s), 1.07 (3H, t, J=7.4 Hz), 1.80-1.98 (2H, m), 2.02 (3H, s), 2.83 (1H, dd, J=14.4, 6.0 Hz), 3.00 (1H, dd, J=14.4, 7.0 Hz), 3.54 (1H, d, J=13.8 Hz), 3.62 (3H, s), 3.74 (1H, d, J=11.4 Hz), 3.88 (1H, d, J=11.4 Hz), 3.90 (3H, s), 3.96 (3H, s), 4.12 (3H, t, J=6.8 Hz), 4.43 (1H, m), 4.57 (1H, d, J=13.8 Hz), 6.31 (1H, s), 6.65 (1H, d, J=1.8 Hz), 6.98 (1H, dd, J=7.8, 2.2 Hz), 7.05-7.21 (3H, m), 7.30-7.40 (2H, m), 7.41-7.47 (2H, m), 7.93 (1H, s).

IR (KBr) 1736, 1678, 1481 cm⁻¹.

Elemental Analysis (C₃₉H₄₃N₂O₁₁Cl.0.5H₂O) Cal'd: C, 61.62; H, 5.83; N, 3.68. Found: C, 61.36; H, 5.79; N, 3.70.

(4) Methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-propoxy-1-benzofuran-2-carboxylate (0.7 g, 0.93 mmol) obtained in Example 151-(3) was dissolved in tetrahydrofuran (7 ml) and ethanol (3 ml), a 2N aqueous sodium hydroxide solution (1.86 ml) was added at room temperature, and the mixture was stirred at 50° C. for 2 hours. Allowing to cool, the mixture was neutralized using 1N hydrochloric acid, concentrated under reduced pressure, ethyl acetate and water were added, and the layers were separated. The organic layer was washed with a 0.4N aqueous sodium hydroxide solution, water, 1N hydrochloric acid, water and an aqueous saturated sodium chloride solution, dried with anhydrous sodium sulfate, and concentrated under reduced pressure. The resulting crude crystals were recrystallized from ethyl acetate (40 ml)-hexane (20 ml), and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-propoxy-1-benzofuran-2-carboxylic acid (0.3 g, yield 46.0%) as white crystals.

m.p. 174.8-176.8° C.

[α]_{D}²²=−110.9° (c=0.47, methanol).

¹H-NMR (200 MHz, DMSO-d₆) δ: 0.77 (3H, s), 0.86 (3H, s), 1.03 (3H, t, J=7.4 Hz), 1.77-1.92 (2H, m), 2.85 (2H, d, J=6.2 Hz), 3.04-3.21 (2H, m), 3.52 (3H, s), 3.68 (1H, d, J=14.0 Hz), 3.84 (3H, s), 4.08 (2H, t, J=76.6 Hz), 4.27-4.40 (2H, m), 4.56 (1H, brs), 6.11 (1H, s), 6.40 (1H, d, J=2.2 Hz), 7.07-7.16 (3H, m), 7.21 (1H, d, J=1.4 Hz), 7.56 (1H, dd, J=8.8, 2.4 Hz), 7.60-7.64 (2H, m), 7.75 (1H, d, J=8.8 Hz), 10.13 (1H, s).

IR (KBr) 3700-2300, 1728, 1651, 1607, 1561, 1481, 1427 cm⁻¹.

Elemental Analysis (C₃₆H₃₉N₂₀₁Cl.0.5H₂O) Cal'd: C, 61.40; H, 5.73; N, 3.98. Found: C, 61.30; H, 5.86; N, 3.98.

### EXAMPLE 152

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-[(1-methylethyl)oxy]-1-benzofuran-2-carboxylic acid

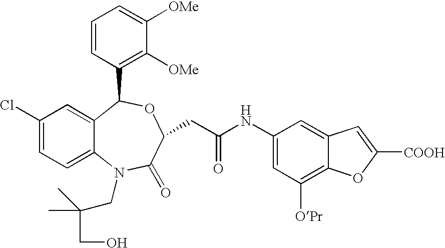

(1) Methyl 7-hydroxy-5-nitro-1-benzofuran-2-carboxylate (1.0 g, 4.22 mmol) was dissolved in N,N-dimethylformamide (20 ml), and potassium carbonate (0.76 g, 5.48 mmol) and 2-iodopropane (0.51 ml, 5.06 mmol) were added at room temperature. After stirred at the same temperature for 14 hours, and stirred at 40° C. for 4 hours. Allowing to cool, water (20 ml) was added to the reaction solution, and the mixture was stirred at room temperature for 1 hour. The crystals were filtered off, washed with methanol: water (4:1) and water, and dried under reduced pressure (50° C.) to obtain methyl 7-[(1-methylethyl)oxy]-5-nitro-1-bezofuran-2-carboxylate (1.05 g, yield 89.2%) as pale brown crystals.

m.p. 137.0-137.9° C.

¹H-NMR (200 MHz, CDCl₃) δ: 1.49 (6H, d, J=6.2 Hz), 4.00 (3H, s), 4.91 (1H, m), 7.62 (1H, s), 7.81 (1H, d, J=1.8 Hz), 8.22 (1H, d, J=1.8 Hz).

IR (KBr) 1725, 1586, 1530, 1346, 1319, 1306 cm⁻¹.

Elemental Analysis (C₁₃H₁₃NO₆) Cal'd: C, 55.91; H, 4.69; N, 5.02. Found: C, 55.77; H, 4.68; N, 5.12.

(2) Methyl 7-[(1-methylethyl)oxy]-5-nitro-1-bezofuran-2-carboxylate (0.80 g, 2.87 mmol) obtained in Example 152-(1) was dissolved in tetrahydrofuran (16 ml), and nitrogen replacement was performed. 10% Palladium carbon (160 mg) was placed therein, and hydrogen was introduced. After stirred at room temperature for 5 hours, the catalyst was filtered off, and the filtrate was concentrated under reduced pressure. Ethyl acetate was added to the resulting residue, 4N hydrogen chloride-ethyl acetate (0.72 ml) was added, the mixture was stirred at room temperature for 1 hour, the crystals were filtered off, and washed with ethyl acetate. Drying under reduced pressure (50° C.) afforded methyl 5-amino-7-[(1-methylethyl)oxy]-1-bezofuran-2-carboxylate hydrochloride (0.76 g, yield 92.8%) as white crystals.

m.p. 221.4-222.0° C.

¹H-NMR (200 MHz, DMSO-d₆) δ: 1.40 (6H, d, J=5.8 Hz), 3.91 (3H, s), 4.79 (1H, m), 7.08 (1H, d, J=1.8 Hz), 7.28 (1H, d, J=1.8 Hz), 7.82 (1H, s).

IR (KBr) 3250-2300, 1752, 1742, 1607, 1561 cm⁻¹.

Elemental Analysis (C₁₃H₁₆NO₄Cl) Cal'd: C, 54.65; H, 5.64; N, 4.90. Found: C, 54.49; H, 5.81; N, 4.86.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepin-3-acetic acid (1.0 g, 1.92 mmol) obtained in Example 1-(1) was dissolved in N,N-dimethylformamide (5 ml) under the argon atmosphere. Triethylamine (0.21 ml, 1.96 mmol) and isobutyl chloroformate (0.28 ml, 2.22 mmol) were added under ice-cooling, and the mixture was stirred at the same temperature for 30 minutes. Methyl 5-amino-7-[(1-methylethyl)oxy]-1-bezofuran-2-carboxylate hydrochloride (0.55 g, 1.92 mmol) obtained in Example 152-(2), and pyridine (0.25 ml, 3.08 mmol) was added dropwise. After stirred at the same temperature for 2 hours, water was added to the reaction solution, and the mixture was extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting crystals were recrystallized from ethyl acetate (15 ml)-hexane (15 ml), and dried under reduced pressure (50° C.) to obtain methyl 5-[[[(3R,5S)-1-(3-acetoxy-2,2-diemthoxypropyl)-7-chloro-5-(2,3-dimethoxyphenyl)-2-oxo-1,2,3,5-tetrahydro-4,1-benzoxazepin-3-yl]acetyl]amino]-7-[(1-methylethyl)oxy]-1-benzofuran-2-carboxylate (0.84 g, yield 58.1%) as white crystals.

m.p. 164.0-167.0° C.

[α]_{D}²²=−101.0° (c=0.30, methanol).

¹H-NMR (200 MHz, CDCl₃) δ: 0.96 (3H, s), 1.03 (3H, s), 1.42 (6H, d, J=5.8 Hz), 2.02 (3H, s), 2.84 (1H, dd, J=14.8, 6.6 Hz), 3.00 (1H, dd, J=14.8, 7.0 Hz), 3.54 (1H, d, J=14.0 Hz), 3.62 (3H, s), 3.73 (1H, d, J=11.4 Hz), 3.88 (1H, d, J=11.4 Hz), 3.90 (3H, s), 3.96 (3H, s), 4.38-4.46 (1H, m), 4.57 (1H, d, J=14.0 Hz), 4.79 (1H, m), 6.31 (1H, s), 6.65 (1H, d, J=2.2 Hz), 6.95-7.01 (1H, m), 7.06-7.21 (3H, m), 7.30-7.40 (2H, m), 7.41-7.46 (2H, m), 7.90 (1H, s).

IR (KBr) 1732, 1676, 1481 cm⁻¹.

Elemental Analysis (C₃₉H₄₃N₂O₁₁Cl.0.5H₂O) Cal'd: C, 61.62; H, 5.83; N, 3.68. Found: C, 61.41; H, 5.71; N, 3.55.

(4) Methyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-[(1-methylethyl)oxy]-1-benzofuran-2-carboxylate (0.7 g, 0.93 mmol) obtained in Example 152-(3) was dissolved in tetrahydrofuran (7 ml) and ethanol (3 ml), a 2N aqueous sodium hydroxide solution (1.86 ml) was added at room temperature, and the mixture was stirred at room temperature for 2 hours. Allowing to cool, the mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (40 ml)-hexane (40 ml), and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-[(1-methylethyl)oxy]-1-benzofuran-2-carboxylic acid (0.61 g, yield 94.2%) as white crystals.

m.p. 188.6-189.7° C.

[α]_{D}²²=−106.7° (c=0.30, methanol).

¹H-NMR (200 MHz, DMSO-d₆) δ: 0.77 (3H, s), 0.86 (3H, s), 1.37 (6H, d, J=5.8 Hz), 2.84 (2H, d, J=6.6 Hz), 3.04-3.20 (2H, m), 3.68 (1H, d, J=13.8 Hz), 3.98 (3H, s), 4.28-4.40 (2H, m), 4.57 (1H, brs), 4.61-4.80 (1H, m), 6.11 (1H, s), 6.41 (1H, d, J=2.2 Hz), 7.07-7.24 (4H, m), 7.51-7.68 (3H, m), 7.75 (1H, d, J=8.8 Hz), 10.12 (1H, s).

IR (KBr) 3700-2300, 1726, 1694, 1572, 1483, 1426 cm⁻¹.

Elemental Analysis (C₃₆H₃₉N₂O₁₀Cl.0.5H₂O) Cal'd: C, 61.40; H, 5.73; N, 3.89. Found: C, 61.27; H, 5.72; N, 3.99.

### EXAMPLE 153

5-[[[(3R,5S)-7-chloro-5-(2,3-dimethoxyphenyl)-7-chloro-1-(3-hydroxy-2,2-dimethylpropyl)-2-oxo-1,2,3,5-tetrahydro-4,1-benzoxazepin-3-yl]acetyl]amino]-3-methoxy-1-benzofuran-2-carboxylic acid

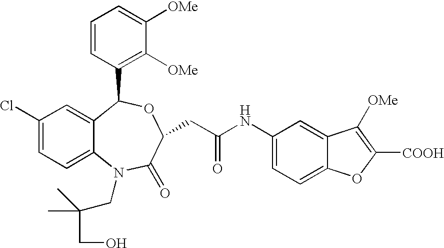

(1) 2-Hydroxy-5-nitrobenzoic acid (15 g, 81.91 mmol) was dissolved in ethanol (150 ml), and concentrated sulfuric acid (3.0 ml) was added. The mixture was stirred under heating at reflux for 72 hours. After allowing to cool, an aqueous saturated sodium bicarbonate solution (50 ml) was added, and water (50 ml) was further added. After stirred at room temperature for 30 minutes, the crystals were filtered, and washed with a 50% aqueous ethanol solution and water. This was drying under reduced pressure (50° C.) afforded ethyl 2-hydroxy-5-nitrobenzoate (14.2 g, yield 82.0%) as pale yellowish white crystals.

m.p. 99.6-99.8° C.

¹H-NMR (200 MHz, CDCl₃): 1.47 (3H, t, J=7.0 Hz), 4.49 (2H, q, J=7.0 Hz), 7.09 (1H, d, J=9.2 Hz), 8.34 (1H, dd, J=9.2, 2.6 Hz), 8.80 (1H, d, J=2.6 Hz).

IR (KBr) 1682, 1626, 1586, 1478, 1345 cm⁻¹.

Elemental Analysis (C₉H₉NO₅) Cal'd: C, 51.19; H, 4.30; N, 6.63. Found: C, 51.13; H, 4.31; N, 6.50.

(2) Ethyl 2-hydroxy-5-nitrobenzoate (13 g, 61.41 mmol) obtained in Example 153-(1) was dissolved in N,N-dimethylformamide (195 ml), and potassium carbonate (15.35 g, 110.53 mmol) and ethyl bromoacetate (8.9 ml, 79.83 mmol) were added. The mixture was stirred at room temperature for 17 hours. Water and ethyl acetate were added to the reaction solution, and the layers were separated. Ethyl acetate was added to the aqueous layer, which was extracted, the organic layers were combined, and washed with water and an aqueous saturated sodium chloride solution. The organic layer was dried with anhydrous sodium sulfate, and concentrated under reduced pressure. The resulting residue was purified by silica gel column chromatography (hexane:ethyl acetate=2:1), the resulting first and second fractions were recrystallized from methanol, respectively, and dried under reduced pressure (50° C.) to obtain ethyl 2-[(2-ethoxy-2-oxoethyl)oxy]-5-nitro-1-benzofuran-2-carboxylate (1.55 g, yield 7.6%) as white crystals and ethyl 2-[(2-ethoxy-2-oxoethyl)oxy]-5-nitrobenzoate (11.7 g, yield 63.9%) as pale yellowish white crystals. Ethyl 2-[(2-ethoxy-2-oxoethyl)oxy]-5-nitro-1-benzofuran-2-carboxylate

m.p. 113.7-113.8° C.

¹H-NMR (200 MHz, CDCl₃) δ: 1.28 (3H, t, J=7.0 Hz), 1.45 (3H, t, J=7.0 Hz), 4.25 (2H, q, J=7.0 Hz), 4.47 (2H, q, J=7.0 Hz), 5.13 (2H, s), 7.59 (1H, d, J=9.0 Hz), 8.38 (1H, dd, J=9.0, 2.6 Hz), 8.78 (1H, d, J=2.6 Hz).

IR (KBr) 1752, 1717, 1537, 1345 cm⁻¹.

Elemental Analysis (C₁₅H₁₅NO₈) Cal'd: C, 53.42; H, 4.48; N, 4.15. Found: C, 53.39; H, 4.36; N, 4.18. Ethyl 2-[(2-ethoxy-2-oxoethyl)oxy]-5-nitrobenzoate

m.p. 77.9-78.0° C.

¹H-NMR (200 MHz, CDCl₃) δ: 1.30 (3H, t, J=7.2 Hz), 1.42 (3H, t, J=8.0 Hz), 4.29 (2H, q, J=7.2 Hz), 4.41 (2H, q, J=8.0 Hz), 4.84 (2H, s), 6.95 (1H, d, J=9.2 Hz), 8.33 (1H, dd, J=9.2, 3.0 Hz), 8.71 (1H, d, J=3.0 Hz).

IR (KBr) 2986, 1732, 1713, 1614, 1588, 1526 cm⁻¹.

Elemental Analysis (C₁₃H₁₅NO₇) Cal'd: C, 52.53; H, 5.09; N, 4.71. Found: C, 52.44; H, 5.12; N, 4.79.

(3) Ethyl 2-[(2-ethoxy-2-oxoethyl)oxy]-5-nitrobenzoate (8.0 g, 26.91 mmol) obtained in Example 153-(2) was dissolved in N,N-dimethylformamide (80 ml), and 1,8-diazabicyclo[5.4.0]-7-undecene (6.0 ml, 40.37 mmol) was added under ice-cooling. A temperature was raised to room temperature, and the mixture was stirred for 5 hours. The mixture was neutralized by the addition of 6N hydrochloric acid under ice-cooling, ethyl acetate was added, and the layers were separated. The organic layer was washed with water and an aqueous saturated sodium chloride solution. The organic layer was dried with anhydrous sodium sulfate, and concentrated under reduced pressure. Diisopropyl ether (300 ml) was added to the resulting crude crystals, which was recrystallized and dried under reduced pressure (50° C.) to obtain ethyl 3-hydroxy-5-nitro-1-benzofuran-2-carboxylate (4.55 g, yield 67.3%) as white crystals.

m.p. 186.1-186.3° C.

¹H-NMR (200 MHz, CDCl₃): 1.48 (3H, t, J=7.4 Hz), 4.52 (2H, q, J=7.4 Hz), 7.59 (1H, d, J=9.4 Hz), 8.40 (1H, dd, J=9.4, 2.6 Hz), 8.71 (1H, d, J=2.6 Hz).

IR (KBr) 3484, 3350, 1725, 1611, 1534, 1346 cm⁻¹.

Elemental Analysis (C₁₁H₁₁NO₆) Cal'd: C, 52.60; H, 3.61; N, 5.58. Found: C, 52.50; H, 3.73; N, 5.47.

(4) Ethyl 3-hydroxy-5-nitro-1-benzofuran-2-carboxylate (1.0 g, 3.98 mmol) obtained in Example 153-(3) was dissolved in N,N-dimethylformamide (10 ml), and 1,8-diazabicyclo[5.4.0]-7-undecene (1.07 ml, 7.17 mmol) and iodomethane (0.28 ml, 5.97 mmol) were added at room temperature. After stirred at the same temperature for 4 hours, 1N hydrochloric acid was added to the reaction solution to neutralize, water and ethyl acetate were added, and the layers were separated. The organic layer was washed with water and an aqueous saturated sodium chloride solution, dried with anhydrous sodium sulfate, and concentrated under reduced pressure. The resulting residue was purified by silica gel column chromatography (hexane:ethyl acetate=2:1), and dried under reduced pressure (50° C.) to obtain ethyl 3-methoxy-5-nitro-1-benzofuran-2-carboxylate (0.85 g, yield 80.5%) as white crystals.

m.p. 90.0-90.4° C.

¹H-NMR (200 MHz, CDCl₃): 1.46 (3H, t, J=7.4 Hz), 4.32 (3H, s), 4.48 (2H, q, J=7.4 Hz), 7.61 (1H, d, J=9.0 Hz), 8.37 (1H, dd, J=9.0, 2.2 Hz), 8.73 (1H, d, J=2.2 Hz).

IR (KBr) 1717, 1534, 1345 cm⁻¹.

Elemental Analysis (C₁₂H₁₁NO₆) Cal'd: C, 54.34; H, 4.18; N, 5.28. Found: C, 54.20; H, 4.36; N, 5.45.

(5) Ethyl 3-methoxy-5-nitro-1-benzofuran-2-carboxylate (0.95 g, 3.58 mmol) obtained in Example 153-(4) was dissolved in ethyl acetate (10 ml), and nitrogen replacement was performed. 10% palladium carbon (95 mg) was placed therein, and hydrogen was introduced. After stirred at room temperature for 3 hours, the catalyst was filtered, and the filtrate was concentrated under reduced pressure. The resulting residue was purified by silica gel column chromatography (hexane:ethyl acetate=1:1) Ethyl acetate was added to the resulting crystals (375 mg), 4N hydrogen chloride-ethyl acetate (0.40 ml) was added thereto, the mixture was stirred at room temperature for 1 hour, the crystals were filtered off, and washed with ethyl acetate. Drying under reduced pressure (50° C.) afforded ethyl 5-amino-3-methoxy-1-benzofuran-2-carboxylate hydrochloride (0.27 g, yield 27.8%) as white crystals.

m.p. 267.4-267.5° C.

¹H-NMR (200 MHz, DMSO-d₆) δ: 1.00 (3H, t, J=7.4 Hz), 3.86 (3H, s), 4.01 (2H, q, J=7.4 Hz), 7.09 (1H, dd, J=8.8, 2.2 Hz), 7.41 (1H, d, J=8.8 Hz), 7.43 (1H, d, J=2.2 Hz).

IR (KBr) 3300-2700, 1713, 1581, 1547 cm⁻¹

Elemental Analysis (C₁₂H₁₄NO₄Cl) Cal'd: C, 53.05; H, 5.19; N, 5.16. Found: C, 52.97; H, 4.89; N, 4.88.

(6) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.38 g, 0.74 mmol) obtained in Example 1-(1) was dissolved in tetrahydrofuran (5 ml), and one droplet of N,N-dimethylformamide was added. Thionyl chloride (0.08 ml, 1.10 mmol) was added at room temperature, the mixture was stirred for 1.5 hours, concentrated under reduced pressure, and dissolved in tetrahydrofuran (5 ml). Ethyl 5-amino-3-methoxy-1-benzofuran-2-carboxylate hydrochloride (0.2 g, 0.74 mmol) obtained in Example 153-(5) was dissolved in tetrahydrofuran (5 ml), and triethylamine (0.26 ml, 1.84 mmol) was added. The previously prepared acid chloride solution was added dropwise at room temperature, the mixture was stirred at the same temperature for 2 hours. Water and ethyl acetate were added to the reaction solution, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=1:1), and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxy-1-benzofuran-2-carboxylate (475 mg, yield 88.2%) as a colorless foam.

[α]_{D}²²=−90.4° (c=0.39, methanol).

¹H-NMR (200 MHz, CDCl₃) δ: 0.96 (3H, s), 1.02 (3H, s), 1.43 (3H, t, J=7.4 Hz), 2.02 (3H, s), 2.86 (1H, dd, J=14.0, 5.8 Hz), 3.02 (1H, dd, J=14.0, 7.4 Hz), 3.54 (1H, d, J=11.4 Hz), 3.62 (3H, s), 3.73 (1H, d, J=11.4 Hz), 3.88 (1H, d, J=11.4 Hz), 3.89 (3H, s), 4.22 (3H, s), 4.37-4.52 (3H, m), 4.57 (1H, d, J=14.4 Hz), 6.31 (1H, s), 6.65 (1H, d, J=1.8 Hz), 6.98 (1H, dd, J=7.2, 1.8 Hz), 7.00-7.21 (2H, m), 7.30-7.45 (4H, m), 8.04 (1H, s), 8.17 (1H, d, J=1.8 Hz).

IR (KBr) 3337, 1750-1650, 1481 cm⁻¹.

Elemental Analysis (C₃₈H₄₁N₂O₁₁Cl.0.2H₂O) Cal'd: C, 61.61; H, 5.63; N, 3.78. Found: C, 61.60; H, 5.40; N, 3.54.

(7) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methoxy-1-benzofuran-2-carboxylate (0.4 g, 0.54 mmol) obtained in Example 153-(6) was dissolved in tetrahydrofuran (4 ml) and ethanol (1 ml), a 2N aqueous sodium hydroxide solution (0.81 ml) was added at room temperature, and the mixture was stirred at the same temperature for 17 hours. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sodium sulfate, and concentrated under reduced pressure. The resulting crude crystals were recrystallized from ethyl acetate-hexane, and dried under reduced pressure (50° C.) to obtain 5-[[[(3R,5S)-7-chloro-5-(2,3-dimethoxyphenyl)-7-chloro-1-(3-hydroxy-2,2-dimethylpropyl)-2-oxo-1,2,3,5-tetrahydro-4,1-benzoxazepin-3-yl]acetyl]amino]-3-methoxy-1-benzofuran-2-carboxylic acid (281 mg, yield 77.6%) as white crystals.

m.p. 175.4-176.3° C.

[α]_{D}²²=−97.1° (c=0.44, methanol).

¹H-NMR (200 MHz, CDCl₃) δ: 0.67 (3H, s), 1.06 (3H, s), 2.89 (1H, dd, J=14.2, 5.8 Hz), 3.05 (1H, dd, J=14.2, 7.4 Hz), 3.19 (1H, d, J=12.0 Hz), 3.41 (1H, d, J=14.6 Hz), 3.62 (3H, s), 3.63 (1H, d, J=12.0 Hz), 3.90 (3H, s), 4.30 (3H, s), 4.41-4.50 (1H, m), 4.50 (1H, d, J=14.6 Hz), 6.21 (1H, s), 6.63 (1H, d, J=1.8 Hz), 6.95-7.03 (1H, m), 7.10-7.20 (2H, m), 7.30-7.50 (4H, m), 8.05 (1H, s), 8.26 (1H, brs).

IR (KBr) 3500-2700, 1661, 1580, 1481 cm⁻¹.

Elemental Analysis (C₃₄H₃₅N₂O₁₀Cl.H₂O) Cal'd: C, 59.61; H, 5.44; N, 4.09. Found: C, 59.42; H, 5.14; N, 4.09.

### EXAMPLE 154

5-[[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-propoxy-1-benzofuran-2-carboxylic acid

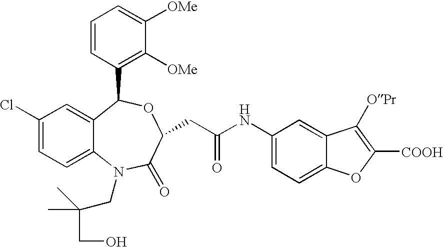

(1) Ethyl 3-hydroxy-5-nitro-1-benzofuran-2-carboxylate (1.0 g, 3.36 mmol) was dissolved in N,N-dimethylformamide (10 ml), and 1,8-diazabicyclo[5.4.0]-7-undecene (1.07 ml, 7.17 mmol) and iodo n-propane (0.58 ml, 5.97 mmol) were added at room temperature. The mixture was stirred at the same temperature for 20 hours, 1N hydrochloric acid was added to the reaction solution to neutralize, water and ethyl acetate were added, and the layers were separated. The organic layer was washed with water and an aqueous saturated sodium chloride solution, dried with anhydrous sodium sulfate, and concentrated under reduced pressure. The resulting residue was purified by silica gel column chromatography (hexane:ethyl acetate=2:1), and dried under reduced pressure (50° C.) to obtain ethyl 5-nitro-3-propoxy-1-benzofuran-2-carboxylate (0.96 g, yield 82.2%) as pale brown crystals.

m.p. 107.0-107.1° C.

¹H-NMR (200 MHz, CDCl₃) δ: 1.11 (3H, t, J=7.4 Hz), 1.45 (3H, t, J=7.0 Hz), 1.81-1.99 (2H, m), 4.40-4.53 (4H, m), 7.61 (1H, d, J=9.2 Hz), 8.36 (1H, dd, J=9.2, 2.2 Hz), 8.69 (1H, d, J=2.2 Hz).

IR (KBr) 1717, 1597, 1526, 1343 cm⁻¹.

Elemental Analysis (C₁₄H₁₅NO₆) Cal'd: C, 57.34; H, 5.16; N, 4.78. Found: C, 57.12; H, 5.20; N, 4.56.

(2) Ethyl 5-nitro-3-propoxy-1-benzofuran-2-carboxylate (0.6 g, 2.05 mmol) obtained in Example 154-(1) was dissolved in ethyl acetate (12 ml), and nitrogen replacement was performed. 10% palladium carbon (60 mg) was placed therein, and hydrogen was introduced. The mixture was stirred at room temperature for 2 hours, the catalyst was filtered, and the filtrate was concentrated under reduced pressure. The resulting residue was purified by silica gel column chromatography (hexane:ethyl acetate=1:l). Ethyl acetate was added to the resulting crystals (565 mg), 4N hydrogen chloride-ethyl acetate (0.54 ml) was added, the mixture was stirred at room temperature for 30 minutes, the crystals were filtered off, and washed with ethyl acetate. Drying under reduced pressure (50° C.) afforded ethyl 5-amino-3-propoxy-1-benzofuran-2-carboxylate hydrochloride (0.57 g, yield 92.3%) as white crystals.

m.p. 183.0-183.3° C.

¹H-NMR (200 MHz, DMSO-d₆) δ: 1.03 (3H, t, J=7.4 Hz), 1.33 (3H, t, J=7.4 Hz), 1.65-1.86 (2H, m), 4.34 (2H, q, J=7.4 Hz), 4.37 (2H, t, J=6.6 Hz), 7.45 (1H, dd, J=9.2, 1.8 Hz), 7.76 (1H, d, J=9.2 Hz), 7.77 (1H, d, J=1.8 Hz).

IR (KBr) 3400-2600, 1726, 1584, 1485 cm⁻¹.

Elemental Analysis (C₁₄H₁₈NO₄Cl) Cal'd: C, 56.10; H, 6.05; N, 4.67. Found: C, 55.95; H, 6.35; N, 4.51.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.5 g, 0.96 mmol) was dissolved in tetrahydrofuran (5 ml), and one droplet of N,N-dimethylformamide was added. Thionyl chloride (0.11 ml, 1.44 mmol) was added at room temperature, the mixture was stirred for 2 hours, concentrated under reduced pressure, and dissolved in tetrahydrofuran (5 ml). Ethyl 5-amino-3-propoxy-1-benzofuran-2-carboxylate hydrochloride (0.29 g, 0.96 mmol) obtained in Example 154-(2) was dissolved in tetrahydrofuran (5 ml), and triethylamine (0.34 ml, 2.40 mmol) was added. The previously prepared acid chloride solution was added dropwise at room temperature, the mixture was stirred at the same temperature for 2 hours. Water and ethyl acetate was added to the reaction solution, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=1:1), and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-propoxy-1-benzofuran-2-carboxylate (579 mg, yield 78.7%) as a colorless foam.

[α]_{D}²²=−86.8° (c=0.23, methanol).

¹H-NMR (200 MHz, CDCl₃) δ: 0.97 (3H, s), 1.02 (3H, s), 1.07 (3H, t, J=7.4 Hz), 1.43 (3H, t, J=7.2 Hz), 2.02 (3H, s), 2.86 (1H, dd, J=14.0, 5.6 Hz), 3.02 (1H, dd, J=14.0, 7.0 Hz), 3.54 (1H, d, J=14.4 Hz), 3.62 (3H, s), 3.73 (1H, d, J=11.0 Hz), 3.88 (1H, d, J=11.0 Hz), 4.00 (3H, s), 4.31-4.50 (5H, m), 4.57 (1H, d, J=14.4 Hz), 6.32 (1H, s), 6.65 (1H, d, J=1.8 Hz), 6.98 (1H, dd, J=7.2, 1.8 Hz), 7.09-7.21 (2H, m), 7.30-7.45 (4H, m), 8.02 (1H, s), 8.11 (1H, d, J=1.4 Hz).

IR (KBr) 3324, 1750-1670, 1481 cm⁻¹.

Elemental Analysis (C₄₀H₄₅N₂O₁₁Cl) Cal'd: C, 62.78; H, 5.93; N, 3.66. Found: C, 62.69; H, 5.76; N, 3.50.

(4) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-propoxy-1-benzofuran-2-carboxylate (0.45 g, 0.59 mmol) obtained in Example 154-(3) was dissolved in tetrahydrofuran (4 ml) and ethanol (1 ml), a 2N aqueous sodium hydroxide solution (0.88 ml) was added at room temperature, and the mixture was stirred at the same temperature for 17 hours. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sulfate, and concentrated under reduced pressure. The resulting residue was purified by silica gel column chromatography (ethyl acetate) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-propoxy-1-benzofuran-2-carboxylic acid (148 mg, yield 36.2%) as a pale yellow foam.

[α]_{D}²²=−107.2° (c=0.14, methanol).

¹H-NMR (200 MHz, CDCl₃) δ: 0.67 (3H, s), 1.00-1.10 (6H, m), 1.78-1.91 (2H, m), 2.90 (1H, dd, J=14.2, 5.8 Hz), 3.06 (1H, dd, J=14.2, 7.8 Hz), 3.20 (1H, d, J=12.2 Hz), 3.41 (1H, d, J=14.4 Hz), 3.60 (3H, s), 3.63 (1H, d, J=12.2 Hz), 3.89 (3H, s), 4.40-4.60 (4H, m), 6.20 (1H, s), 6.62 (1H, s), 6.98 (1H, dd, J=6.8, 2.6 Hz), 7.05-7.21 (2H, m), 7.31-7.43 (4H, m), 8.18 (1H, s), 8.32 (1H, brs).

IR (KBr) 3600-2700, 1659, 1574, 1481 cm⁻¹.

Elemental Analysis (C₃₆H₃₉N₂O₁₀Cl.0.2H₂O) Cal'd: C, 62.20; H, 5.65; N, 4.03. Found: C, 61.60; H, 5.75; N, 3.77.

### EXAMPLE 155

5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-[(carboxymethyl)oxy]-1-benzofuran-2-carboxylic acid

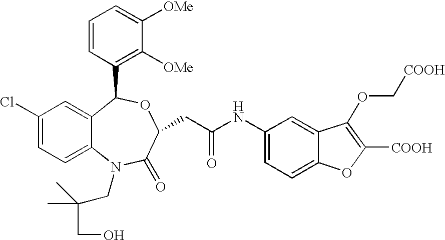

(1) Ethyl 3-[(carboxymethyl)oxy]-5-nitro-1-benzofuran-2-carboxylate (0.74 g, 2.23 mmol) was dissolved in ethyl acetate (12 ml), and nitrogen replacement was performed. 10% palladium carbon (74 mg) was placed therein, and hydrogen was introduced. After the mixture was stirred at room temperature for 3 hours, the catalyst was filtered, and the filtrate was concentrated under reduced pressure. Ethyl acetate was added to the resulting residue, 4N hydrogen chloride-ethyl acetate (0.56 ml) was added, the mixture was stirred at room temperature for 1 hour, the crystals were filtered off, and washed with ethyl acetate. Drying under reduced pressure (50° C.) afforded ethyl 5-amino-3-[(carboxymethyl)oxy]-1-benzofuran-2-carboxylate hydrochloride (0.53 g, yield 69.3%) as white crystals.

m.p. 152.7-154.6° C.

¹H-NMR (200 MHz, DMSO-d₆) δ: 1.20 (3H, t, J=7.6 Hz), 1.35 (3H, t, J=7.0 Hz), 4.17 (2H, q, J=7.6 Hz), 4.36 (2H, q, J=7.0 Hz), 5.16 (2H, s), 7.52 (1H, dd, J=8.8, 1.8 Hz), 7.75-7.83 (2H, m).

IR (KBr) 3250-2600, 1767, 1753, 1732, 1583 cm⁻¹.

Elemental Analysis (C₁₅H₁₈NO₆Cl) Cal'd: C, 52.41; H, 5.28; N, 4.07. Found: C, 52.23; H, 5.28; N, 3.98.

(2) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.6 g, 1.16 mmol) obtained in Example 1-(1) was dissolved in (6 ml), and one droplet of N,N-dimethylformamide was added. Thionyl chloride (0.11 ml, 1.51 mmol) was added at room temperature, the mixture was stirred for 2 hours, concentrated under reduced pressure, and dissolved in tetrahydrofuran (5 ml). Ethyl 5-amino-3-[(carboxymethyl)oxy]-1-benzofuran-2-carboxylate hydrochloride (0.4 g, 1.16 mmol) obtained in Example 155-(1) was dissolved in tetrahydrofuran (5 ml), and triethylamine (0.41 ml, 2.91 mmol) was added. The previously prepared acid chloride solution was added dropwise at room temperature, and the mixture was stirred at the same temperature for 1 hour. Water and ethyl acetate were added to the reaction solution, the layers were separated, and the organic layer was washed with water and an aqueous saturated sodium chloride solution. Organic layer was dried with anhydrous sodium sulfate, and concentrated under reduced pressure. The resulting residue was purified by silica gel column chromatography (hexane:ethyl acetate=1:l), and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-[(carboxymethyl)oxy]-1-benzofuran-2-carboxylate (476 mg, yield 51.0%) as a colorless foam.

[α]_{D}²²=−81.4° (c=0.40, methanol).

¹H-NMR (200 MHz, CDCl₃) δ: 0.96 (3H, s), 1.01 (3H, s), 1.25 (3H, t, J=7.4 Hz), 1.43 (3H, t, J=7.4 Hz), 1.99 (3H, s), 2.88 (1H, dd, J=14.8, 5.0 Hz), 3.09 (1H, dd, J=14.8, 7.6 Hz), 3.57 (1H, d, J=14.4 Hz), 3.61 (3H, s), 3.78 (1H, d, J=11.4 Hz), 3.86 (1H, d, J=11.4 Hz), 3.88 (3H, s), 4.24 (2H, q, J=7.4 Hz), 4.39-4.51 (3H, m), 4.57 (1H, d, J=14.4 Hz), 5.01 (2H, s), 6.31 (1H, s), 6.65 (1H, s), 6.97 (1H, d, J=7.4 Hz), 7.00-7.24 (2H, m), 7.27-7.45 (4H, m), 8.05 (1H, s), 8.56 (1H, s).

IR (KBr) 3295, 1760-1650, 1559, 1481 cm⁻¹.

Elemental Analysis (C₄₁H₄₅N₂O₁₃Cl) Cal'd: C, 60.85; H, 5.60; N, 3.46. Found: C, 60.82; H, 5.63; N, 3.38.

(3) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-[(carboxymethyl)oxy]-1-benzofuran-2-carboxylate (0.3 g, 0.37 mmol) obtained in Example 155-(2) was dissolved in tetrahydrofuran (3 ml) and ethanol (1 ml), a 2N aqueous sodium hydroxide solution (0.56 ml) was added at room temperature, and the mixture was stirred at the same temperature for 1 hour. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sodium sulfate, and concentrated under reduced pressure. The resulting residue was recrystallized from methanol-ethyl acetate, and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-[(carboxymethyl)oxy]-1-benzofuran-2-carboxylic acid (190 mg, yield 72.1%) as white crystals.

m.p. 193.0-195.5° C.

[α]_{D}²²=−98.6° (c=0.28, methanol).

¹H-NMR (200 MHz, DMSO-d₆) δ: 0.76 (3H, s), 0.86 (3H, s), 2.80-2.91 (2H, m), 3.05-3.20 (2H, m), 3.70 (1H, d, J=13.2 Hz), 3.84 (3H, s), 4.28-4.41 (2H, m), 4.59 (1H, brs), 4.75 (2H, s), 6.11 (1H, s), 6.41 (1H, d, J=2.2 Hz), 7.10-7.20 (3H, m), 7.50-7.60 (3H, m), 7.77 (1H, d, J=8.8 Hz), 8.31 (1H, d, J=1.2 Hz), 10.32 (1H, s).

IR (KBr) 3800-2600, 1750-1500, 1481 cm⁻¹.

Elemental Analysis (C₃₅H₃₅N₂O₁₂Cl) Cal'd: C, 59.12; H, 4.96; N, 3.94. Found: C, 59.23; H, 5.23; N, 3.78.

### EXAMPLE 156

5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-[(1-methylethyl)oxy]-1-benzofuran-2-carboxylic acid

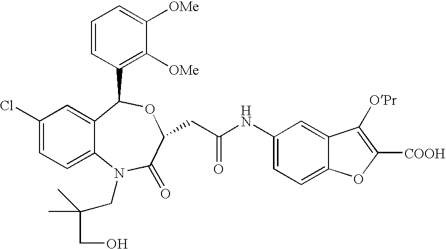

(1) Ethyl 3-hydroxy-5-nitro-1-benzofuran-2-carboxylate (1.0 g, 3.98 mmol) was dissolved in N,N-dimethylformamide (10 ml), and 1,8-diazabicyclo[5.4.0]-7-undecene (1.07 ml, 7.17 mmol) and 2-iodopropane (0.58 ml, 5.97 mmol) were added at room temperature. The mixture was stirred at the same temperature for 20 hours, 1N hydrochloric acid was added to the reaction solution to neutralize, water and ethyl acetate were added, and the layers were separated. The organic layer was washed with water and an aqueous saturated sodium chloride solution, dried with anhydrous sodium sulfate, and concentrated under reduced pressure. The resulting residue were purified by silica gel column chromatography (hexane:ethyl acetate=2:1), and dried under reduced pressure (50° C.) to obtain ethyl 3-[(1-methylethyl)oxy]-5-nitro-1-benzofuran-2-carboxylate (0.76 g, yield 65.1%) as white crystals.

m.p. 122.3-122.4° C.

¹H-NMR (200 MHz, CDCl₃): 1.43 (3H, s), 1.45 (3H, t, J=7.2 Hz), 1.46 (3H, s), 4.47 (2H, q, J=7.2 Hz), 4.95 (1H, m), 7.61 (1H, d, J=9.0 Hz), 8.36 (1H, dd, J=9.0, 2.2 Hz), 8.63 (1H, d, J=2.2 Hz).

IR (KBr) 1717, 1574, 1532, 1345 cm⁻¹.

Elemental Analysis (C₁₄H₁₅NO₆) Cal'd: C, 57.34; H, 5.16; N, 4.78. Found: C, 57.06; H, 5.17; N, 4.68.

(2) Ethyl 3-[(1-methylethyl)oxy]-5-nitro-1-benzofuran-2-carboxylate (0.67 g, 2.28 mmol) obtained in Example 156-(1) was dissolved in ethyl acetate (12 ml), and nitrogen replacement was performed. 10% Palladium carbon (67 mg) was placed therein, and hydrogen was introduced. After stirred at room temperature for 2 hours, the catalyst was filtered, and the filtrate was concentrated under reduced pressure. The resulting residue was purified by silica gel column chromatography (hexane:ethyl acetate=1:1). Ethyl acetate was added to the resulting brown oil (596 mg), 4N hydrogen chloride-ethyl acetate (0.57 ml) was added, the mixture was stirred at room temperature for 30 minutes, the crystals were filtered off, and washed with ethyl acetate. Drying under reduced pressure (50° C.) to obtain ethyl 5-amino-3-[(1-methylethyl)oxy]-1-benzofuran-2-carboxylate hydrochloride (0.53 g, yield 79.4%) as white crystals.

m.p. 213.9-214.0° C.

¹H-NMR (200 MHz, DMSO-d₆): 1.33 (3H, s), 1.34 (3H, t, J=7.2 Hz), 1.38 (3H, s), 4.35 (2H, q, J=7.2 Hz), 4.79 (1H, m), 7.53 (1H, dd, J=8.8, 2.2 Hz), 7.80 (1H, d, J=8.8 Hz), 7.82 (1H, d, J=2.2 Hz).

IR (KBr) 3200-2600, 1719, 1595 cm⁻¹.

Elemental Analysis (C₁₄H₁₈NO₄Cl) Cal'd: C, 56.10; H, 6.05; N, 4.67. Found: C, 56.14; H, 6.13; N, 4.67.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.5 g, 0.96 mmol) obtained in Example 1-(1) was dissolved in tetrahydrofuran (5 ml), and one droplet of N,N-dimethylformamide was added. Thionyl chloride (0.11 ml, 1.44 mmol) was added at room temperature, the mixture was stirred for 2 hours, concentrated under reduced pressure, and dissolved in tetrahydrofuran (5 ml). Ethyl 5-amino-3-[(1-methylethyl)oxy]-1-benzofuran-2-carboxylate hydrochloride (0.29 g, 0.96 mmol) obtained in Example 156-(2) was dissolved in tetrahydrofuran (5 ml), and triethylamine (0.34 ml, 2.40 mmol) was added. The previously prepared acid chloride solution was added dropwise at room temperature, and the mixture was stirred at the same temperature for 2 hours. Water and ethyl acetate were added to the reaction solution, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=1:1), and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-[(1-methylethyl)oxy]-1-benzofuran-2-carboxylate (459 mg, yield 62.4%) as a colorless foam.

[α]_{D}²²=−89.0° (c=0.39, methanol).

¹H-NMR (200 MHz, CDCl₃) δ: 0.96 (3H, s), 1.02 (3H, s), 1.38 (3H, s), 2.03 (3H, s), 1.40 (3H, s), 1.43 (3H, t, J=7.4 Hz), 2.02 (3H, s), 2.86 (1H, dd, J=13.8, 5.8 Hz), 3.02 (1H, dd, J=13.8, 7.2 Hz), 3.54 (1H, d, J=14.0 Hz), 3.62 (3H, s), 3.73 (1H, d, J=11.0 Hz), 3.88 (1H, d, J=11.0 Hz), 4.00 (3H, s), 4.35-4.50 (3H, m), 4.57 (1H, d, J=14.0 Hz), 4.84 (1H, m), 6.14 (1H, s), 6.65 (1H, d, J=1.8 Hz), 6.99 (1H, dd, J=7.2, 1.8 Hz), 7.10-7.21 (2H, m), 7.30-7.46 (4H, m), 8.00-8.06 (2H, m).

IR (KBr) 3330, 1750-1670, 1481 cm⁻¹.

Elemental Analysis (C₄₀H₄₅N₂O₁₁Cl) Cal'd: C, 62.78; H, 5.93; N, 3.66. Found: C, 62.60; H, 6.14; N, 3.50.

(4) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-[(1-methylethyl)oxy]-1-benzofuran-2-carboxylate (0.35 g, 0.46 mmol) obtained in Example 156-(3) was dissolved in tetrahydrofuran (3.5 ml) and ethanol (1 ml), a 2N aqueous sodium hydroxide solution (0.68 ml) was added at room temperature, and the mixture was stirred at 40° C. for 3.5 hours. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sodium sulfate, and concentrated under reduced pressure. The resulting residue was recrystallized from ethyl acetate-hexane, and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-[(1-methylethyl)oxy]-1-benzofuran-2-carboxylic acid (183 mg, yield 57.6%) as white crystals.

m.p. 174.2-174.9° C.

[α]_{D}²²=−93.8° (c=0.39, methanol).

¹H-NMR (200 MHz, DMSO-d₆) δ: 0.77 (3H, s), 0.86 (3H, s), 1.29 (3H, s), 1.32 (3H, s), 2.86 (2H, d, J=6.6 Hz), 3.01-3.21 (2H, m), 3.52 (3H, s), 3.67 (1H, d, J=14.2 Hz), 3.84 (3H, s), 4.28-4.40 (2H, m), 4.56 (1H, brs), 4.79 (1H, m), 6.11 (1H, s), 6.40 (1H, d, J=2.6 Hz), 7.07-7.61 (3H, m), 7.75 (1H, d, J=8.8 Hz), 8.09 (1H, d, J=1.4 Hz), 10.23 (1H, s).

IR (KBr) 3700-2300, 1686, 1655, 1586, 1551, 1481 cm⁻¹.

Elemental Analysis (C₃₆H₃₉N₂O₁₀Cl.0.1H₂O) Cal'd: C, 62.04; H, 5.67; N, 4.02. Found: C, 61.84; H, 5.69; N, 3.81.

### EXAMPLE 157

5-[[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1H-indole-2-carboxylic acid

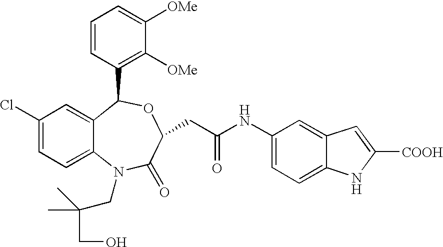

(1) Ethyl 5-nitro-1H-indole-2-carboxylate (1.5 g, 6.41 mmol) was dissolved in ethyl acetate, and nitrogen replacement was performed. 10% palladium carbon (300 mg) was placed therein, and hydrogen was introduced. The mixture was stirred at room temperature for 3 hours, the catalyst was filtered, and the filtrate was concentrated under reduced pressure. The resulting crude crystals were recrystallized from ethyl acetate-hexane, and dried under reduced pressure (50° C.) to obtain ethyl 5-amino-1H-indole-2-carboxylate (865 mg, yield 66.1%) as a brown crystal.

m.p. 131.6-132.6° C.

¹H-NMR (200 MHz, CDCl₃) δ: 1.40 (3H, t, J=7.4 Hz), 4.39 (2H, q, J=7.4 Hz), 6.81 (1H, dd, J=8.8, 2.2 Hz), 6.94 (1H, d, J=2.2 Hz), 7.00-7.05 (1H, m), 7.24 (1H, d, J=8.8 Hz).

IR (KBr) 3400-3090, 1696, 1532, 1235 cm⁻¹.

Elemental Analysis (C₁₁H₁₂N₂O₂) Cal'd: C, 64.69; H, 5.92; N, 13.72. Found: C, 64.68; H, 5.96; N, 13.82.

(2) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in tetrahydrofuran (10 ml), and one droplet of N,N-dimethylformamide was added. Thionyl chloride (0.21 ml, 2.89 mmol) was added at room temperature, the mixture was stirred for 2 hours, concentrated under reduced pressure, and dissolved in tetrahydrofuran (5 ml). Ethyl 5-amino-1H-indole-2-carboxylate (0.39 g, 1.92 mmol) obtained in Example 157-(1) was dissolved in tetrahydrofuran (10 ml), and triethylamine (0.4 ml, 2.89 mmol) was added. The previously prepared acid chloride solution was added dropwise at room temperature, and the mixture was stirred at the same temperature for 3 hours. Water and ethyl acetate were added to the reaction solution, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=1:1), the resulting crude crystals were recrystallized from ethyl acetate-hexane, and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1H-indole-2-carboxylate (860 mg, yield 63.3%) as white crystals.

m.p. 200.5-200.6° C.

[α]_{D}²²=−90.9° (c=0.32, methanol).

¹H-NMR (200 MHz, CDCl₃) δ: 0.96 (3H, s), 1.03 (3H, s), 1.42 (3H, t, J=7.4 Hz), 2.03 (3H, s), 2.85 (1H, dd, J=14.2, 5.8 Hz), 3.02 (1H, dd, J=14.2, 6.8 Hz), 3.54 (1H, d, J=13.8 Hz), 3.62 (3H, s), 3.74 (1H, d, J=11.0 Hz), 3.88 (1H, d, J=11.0 Hz), 3.89 (3H, s), 4.30-4.50 (3H, m), 4.57 (1H, d, J=13.8 Hz), 6.31 (1H, s), 6.64 (1H, d, J=1.8 Hz), 6.98 (1H, dd, J=7.6, 1.8 Hz), 7.07-7.24 (3H, m), 7.28-7.39 (4H, m), 7.85 (1H, s), 7.96 (1H, s), 8.86 (1H, brs).

IR (KBr) 3343, 1723, 1653, 1481 cm⁻¹.

Elemental Analysis (C₃₇H₄₀N₃O₉Cl) Cal'd: C, 62.93; H, 5.71; N, 5.95. Found: C, 62.98; H, 5.54; N, 5.65.

(3) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1H-indole-2-carboxylate (0.5 g, 0.71 mmol) obtained in Example 157-(2) was dissolved in tetrahydrofuran (5 ml) and ethanol (1.5 ml), a 2N aqueous sodium hydroxide solution (1.06 ml) was added at room temperature, and the mixture was stirred at 45° C. for 4 hours. The mixture was neutralized using 1N hydrochloric acid, concentrated under reduced pressure, ethyl acetate and water were added, and the layers were separated. The organic layer was washed with an aqueous saturated sodium chloride solution, dried with anhydrous sodium sulfate, and concentrated under reduced pressure. The resulting residue was recrystallized from ethyl acetate-hexane, and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1H-indole-2-carboxylic acid (860 mg, yield 63.3%) as white crystals.

m.p. 200.5-200.6° C.

[α]_{D}²²=−107.2° C. (c=0.29, methanol).

¹H-NMR (200 MHz, CDCl₃) δ: 0.63 (3H, s), 1.01 (3H, s), 2.80-3.00 (1H, m), 3.01-3.15 (1H, m), 3.20 (1H, d, J=11.6 Hz), 3.33 (1H, d, J=13.8 Hz), 3.57 (3H, s), 3.64 (1H, d, J=11.6 Hz), 3.86 (3H, s), 4.40-4.60 (2H, m), 6.18 (1H, s), 6.59 (1H, s), 6.95 (1H, d, J=7.6 Hz), 7.10 (1H, d, J=7.6 Hz), 7.12-7.40 (6H, m), 7.78 (1H, brs), 8.14 (1H, brs), 9.38 (1H, brs).

IR (KBr) 3343, 1723, 1653, 1481 cm⁻¹.

Elemental Analysis (C₃₃H₃₄N₃O₈Cl.H₂O) Cal'd: C, 60.60; H, 5.55; N, 6.42. Found: C, 60.54; H, 5.51; N, 6.18.

### EXAMPLE 158

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-benzothiophene-2-carboxylic acid

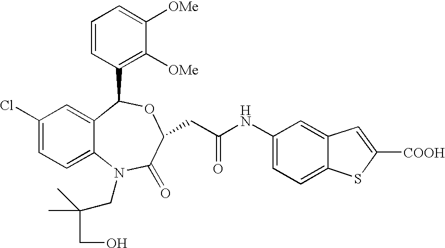

(1) 2-Fluoro-5-nitrobenzaldehyde (4.5 g, 26.61 mmol) was dissolved in N,N-dimethylformamide (45 ml), and potassium carbonate (7.36 g, 53.22 mmol) was added. Ethyl thioglycolate (3.06 ml, 27.94 mmol) was added at room temperature, and the mixture was stirred for 1 hour. The mixture was neutralized using 6N hydrochloric acid under ice-cooling, and extracted with ethyl acetate. The organic layers were combined, and washed with water and an aqueous saturated sodium chloride solution. The organic layer was dried with anhydrous sodium sulfate, and concentrated under reduced pressure. Methanol was added to the resulting crude crystals, the mixture was stirred at room temperature for 2 hours, and the crystals were filtered. Drying under reduced pressure (50° C.) afforded ethyl 5-nitro-1-benzothiophene-2-carboxylate (6.36 g, yield 95.1%) as white crystals.

m.p. 168.6-168.7° C.

¹H-NMR (200 MHz, CDCl₃): 1.44 (3H, t, J=7.0 Hz), 4.45 (2H, q, J=7.0 Hz), 8.00 (1H, d, J=9.0 Hz), 8.19 (1H, s), 8.31 (1H, dd, J=9.0, 2.2 Hz), 8.79 (1H, d, J=2.2 Hz).

IR (KBr) 1701, 1532, 1348, 1304 cm⁻¹.

Elemental Analysis (C₁₁H₉NO₄S) Cal'd: C, 52.58; H, 3.61; N, 5.57. Found: C, 52.33; H, 3.53; N, 5.58.

(2) Ethyl 5-nitro-1-benzothiophene-2-carboxylate (2.5 g, 9.95 mmol) obtained in Example 158-(1) was dissolved in tetrahydrofuran (50 ml), nitrogen replacement was performed. 10% palladium carbon (1.0 g) was placed therein, hydrogen was introduced. The mixture was stirred at room temperature for 3 hours, the catalyst was filtered, and the filtrate was concentrated under reduced pressure. Ethyl acetate was added to the resulting residue, 4N hydrogen chloride-ethyl acetate (4.29 ml) was added, the mixture was stirred at room temperature for 1 hour, the crystals were filtered, and washed with ethyl acetate. Drying under reduced pressure (50° C.) afforded ethyl 5-amino-1-benzothiophene-2-carboxylate hydrochloride (2.24 g, yield 87.8%) as white crystals.

m.p. 205.0-251.1° C.

¹H-NMR (200 MHz, CD₃OD): 1.41 (3H, t, J=7.4 Hz), 4.42 (2H, q, J=7.4 Hz), 7.49 (1H, dd, J=8.6, 2.2 Hz) 8.00 (1H, d, J=2.2 Hz), 8.14 (1H, d, J=8.6 Hz), 8.17 (1H, s).

IR (KBr) 3250-2330, 1721, 1707, 1532, 1514, 1294 cm⁻¹.

Elemental Analysis (C₁₁H₁₂NO₂SCl) Cal'd: C, 51.26; H, 4.69; N, 5.43. Found: C, 51.28; H, 4.77; N, 5.51.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in tetrahydrofuran (10 ml), and one droplet of N,N-dimethylformamide was added. Thionyl chloride (0.21 ml, 2.89 mmol) was added at room temperature, the mixture was stirred for 2 hours, concentrated under reduced pressure, and dissolved in tetrahydrofuran (5 ml). Ethyl 5-amino-1-benzothiophene-2-carboxylate (0.5 g, 1.92 mmol) obtained in Example 158-(2) was suspended in tetrahydrofuran (10 ml), and triethylamine (0.67 ml, 4.81 mmol) was added. The previously prepared acid chloride solution was added dropwise at room temperature, and the mixture was stirred at the same temperature for 2 hours. Water and ethyl acetate were added to the reaction solution, the layers were separated, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=2:1), the resulting crude crystals were recrystallized from ethyl acetate-hexane, and dried under reduced pressure (50° C.) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-benzothiophene-2-carboxylate (1.0 g, yield 71.9%) as white crystals.

[α]_{D}²²=−79.6° (c=0.43, methanol).

¹H-NMR (200 MHz, CDCl₃) δ: 0.97 (3H, s), 1.02 (3H, s), 1.42 (3H, t, J=7.0 Hz), 2.02 (3H, s), 2.88 (1H, dd, J=13.8, 5.4 Hz), 3.04 (1H, dd, J=13.8, 7.2 Hz), 3.55 (1H, d, J=14.4 Hz), 3.62 (3H, s), 3.74 (1H, d, J=11.4 Hz), 3.88 (1H, d, J=11.4 Hz), 3.90 (3H, s), 4.41 (2H, q, J=7.0 Hz), 4.38-4.50 (1H, m), 4.57 (1H, d, J=14.4 Hz), 6.32 (1H, s), 6.65 (1H, d, J=1.8 Hz), 6.99 (1H, dd, J=7.6, 2.2 Hz), 7.13 (1H, t, J=7.6 Hz), 7.20 (1H, dd, J=7.6, 2.2 Hz), 7.30-7.38 (2H, m), 7.43 (1H, dd, J=8.8, 2.2 Hz), 7.76 (1H, d, J=8.8 Hz), 7.98 (1H, s), 8.11 (1H, s), 8.21 (1H, d, J=2.2 Hz).

IR (KBr) 3328, 1750-1650, 1481, 1283, 1246 cm⁻¹.

Elemental Analysis (C₃₇H₃₉N₂O₉Cls) Cal'd: C, 61.45; H, 5.44; N, 3.87. Found: C, 61.15; H, 5.64; N, 3.91.

(4) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-benzothiophene-2-carboxylate (0.7 g, 0.97 mmol) obtained in Example 158-(3) was dissolved in tetrahydrofuran (7 ml) and ethanol (2 ml), a 2N aqueous sodium hydroxide solution (1.45 ml) was added at room temperature, and the mixture was stirred at 40° C. for 4 hours. After allowing to cool, the mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (40 ml)-hexane (20 ml) and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-benzothiophene-2-carboxylic acid (0.377 g, yield 59.6%) as white crystals.

m.p. 180.0-181.0° C.

[α]_{D}²²=−91.7° (c=0.30, methanol).

¹H-NMR (200 MHz, CDCl₃) δ: 0.67 (3H, s), 1.06 (3H, s), 2.91 (1H, dd, J=14.6, 5.6 Hz), 3.10 (1H, dd, J=14.6, 8.0 Hz), 3.21 (1H, d, J=12.8 Hz), 3.41 (1H, d, J=14.6 Hz), 3.61 (3H, s), 3.64 (1H, d, J=14.6 Hz), 3.89 (3H, s), 4.45-4.60 (2H, m), 6.21 (1H, s), 6.63 (1H, s), 6.99 (1H, dd, J=7.4, 2.6 Hz), 7.10-7.22 (1H, d, J=8.8 Hz), 7.94 (1H, s), 8.10 (1H, s), 8.24 (1H, s).

IR (KBr) 3600-2400, 1740-1600, 1524, 1481, 1281 cm⁻¹.

Elemental Analysis (C₃₃H₃₃N₂O₈Cls.H₂O) Cal'd: C, 59.06; H, 5.26; N, 4.17. Found: C, 59.27; H, 5.24; N, 3.99.

### EXAMPLE 159

3-[5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-benzofuran-2-yl]propanoic acid

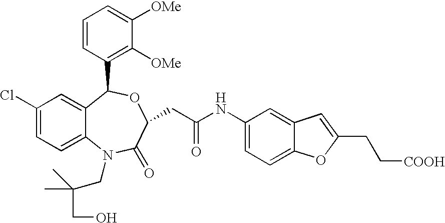

(1) 5-Nitro-1-benzofuran-2-carboxylic acid (4.0 g, 19.31 mmol) was dissolved in tetrahydrofuran (40 ml), and N-methylmorpholine (2.55 ml, 23.17 mmol) was added. Ethyl chlorocarbonate (2.22 ml, 23.17 mmol) was added dropwise under ice-cooling, and the mixture was stirred for 30 minutes. A solution of sodium borohydride (2.19 g, 57.93 mmol) in N,N-dimethylformamide (40 ml) was added dropwise at −40° C., and the mixture was stirred at the same temperature for 2 hours. 1N hydrochloric acid was added, followed by extraction with ethyl acetate. The organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=1:1), and dried under reduced pressure (50° C.) to obtain (5-nitro-1-benzofuran-2-yl)methanol (3.4 g, yield 91.2%) as a pale yellow crystal.

m.p. 115.3-116.3° C.

¹H-NMR (200 MHz, CDCl₃) δ: 2.04 (1H, t, J=6.2 Hz), 4.84 (2H, d, J=6.2 Hz), 6.83 (1H, s), 7.55 (1H, d, J=9.0 Hz), 8.23 (1H, dd, J=9.0, 2.2 Hz), 8.50 (1H, d, J=2.2 Hz).

IR (KBr) 3517, 3108, 1507, 1352 cm⁻¹.

Elemental Analysis (C₉H₇NO₄) Cal'd: C, 55.96; H, 3.65; N, 7.25. Found: C, 55.72; H, 3.49; N, 7.35.

(2) (5-Nitro-1-benzofuran-2-yl)methanol (0.19 g, 0.98 mmol) obtained in Example 159-(1) was dissolved in tetrahydrofuran (4 ml). Manganese dioxide (0.86 g, 9.84 mmol) was added at room temperature, and the mixture was stirred at 60° C. for 15 hours. The insolubles were filtered using Celite, the filtrate was concentrated under reduced pressure, the resulting residue was purified by silica gel column chromatography (hexane:ethyl acetate=3:1), and dried under reduced pressure (50° C.) to obtain 2-formyl-5-nitro-1-benzofuran (0.16 g, yield 85.0%) as pale yellow crystals.

¹H-NMR (200 MHz, CDCl₃) δ: 7.72 (1H, s), 7.75 (1H, t, J=9.4 Hz), 8.45 (1H, d, J=9.4, 2.2 Hz), 8.74 (1H, d, J=2.2 Hz), 9.97 (1H, s).

IR (KBr) 1696, 1524, 1350 cm⁻¹.

Elemental Analysis (C₉H₅NO₄) Cal'd: C, 56.55; H, 2.64; N, 7.33. Found: C, 56.58; H, 2.82; N, 7.51.

(3) 2-Formyl-5-nitro-1-benzofuran (0.3 g, 1.57 mmol) was dissolved in tetrahydrofuran (9 ml), and (carboethoxymethylene)triphenylphosphorane (0.57 g, 1.64 mmol) was added at room temperature. After stirred for 1 hour, water was added, the mixture was extracted with ethyl acetate, and the organic layer was washed with water and an aqueous saturated sodium chloride solution. The organic layer was dried with anhydrous sodium sulfate, and concentrated under reduced pressure. The resulting residue was purified by silica gel column chromatography (hexane:ethyl acetate=3:1), and dried under reduced pressure (50° C.) to obtain ethyl (E)-3-(5-nitro-1-benzofuran-2-yl)-2-propenoate (388 mg, yield 94.6%) as white crystals.

¹H-NMR (200 MHz, CDCl₃) δ: 1.36 (3H, t, J=7.2 Hz), 4.30 (2H, q, J=7.2 Hz), 6.66 (1H, d, J=15.8 Hz), 7.06 (1H, s), 7.56 (1H, d, J=15.8 Hz), 7.58 (1H, d, J=8.4 Hz), 8.29 (1H, dd, J=8.4, 2.6 Hz), 8.53 (1H, d, J=2.6 Hz).

IR (KBr) 1713, 1530, 1348 cm⁻¹.

Elemental Analysis (C₁₃H₁₁NO) Cal'd: C, 59.77; H, 4.24; N, 5.36. Found: C, 59.82; H, 4.08; N, 5.38.

(4) Ethyl (E)-3-(5-nitro-1-benzofuran-2-yl)-2-propenoate (0.38 g, 1.46 mmol) obtained in Example 159-(3) was dissolved in tetrahydrofuran (8 ml), and nitrogen replacement was performed. 10% palladium carbon (60 mg) was placed therein, and hydrogen was introduced. The mixture was stirred at room temperature for 4.5 hours, the catalyst was filtered, and the filtrate was concentrated under reduced pressure. The resulting residue was purified by silica gel column chromatography (hexane:ethyl acetate=2:1), ethyl acetate was added to the resulting crystals (231 mg), 4N hydrogen chloride-ethyl acetate (0.28 ml) was added, the mixture was stirred at room temperature, the crystals were filtered, and washed with ethyl acetate. Drying under reduced pressure (50° C.) afforded ethyl 3-(5-amino-1-benzofuran-2-yl)propanoate hydrochloride (0.23 g, yield 58.6%) as white crystals.

mp183.1-185.5° C.

¹H-NMR (200 MHz, DMSO-d₆) δ: 1.70 (3H, t, J=7.0 Hz), 2.76 (2H, t, J=7.0 Hz), 3.06 (2H, t, J=7.0 Hz), 4.08 (2H, q, J=7.0 Hz), 6.71 (1H, s), 7.21 (1H, dd, J=8.4, 2.2 Hz), 7.54 (1H, d, J=2.2 Hz), 7.61 (1H, d, J=8.4

IR (KBr) 3300-2300, 1738, 1582, 1480 cm⁻¹.

Elemental Analysis (C₁₃H₁₆NO₃Cl) Cal'd: C, 57.89; H, 5.98; N, 5.19. Found: C, 57.97; H, 6.02; N, 5.05.

(5) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.35 g, 0.67 mmol) obtained in Example 1-(1) was dissolved in N,N-dimethylformamide (5 ml) under the argon atmosphere. Triethylamine (0.1 ml, 0.69 mmol) and isobutyl chloroformate (0.1 ml, 0.77 mmol) were added under ice-cooling, and the mixture was stirred at the same temperature for 30 minutes. Ethyl 3-(5-amino-1-benzofuran-2-yl)propanoate hydrochloride (0.18 g, 0.67 mmol) obtained in Example 159-(4) was added, and pyridine (0.087 ml, 1.08 mmol) was added dropwise. The mixture was stirred at the same temperature for 2 hours, water was added to the reaction solution, and the mixture was extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=3:2) to obtain ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-benzofuran-2-yl]propanoate (0.45 g, yield 90.9%) as a colorless foam.

[α]_{D}²²=−111.2° (c=0.24, methanol).

¹H-NMR (200 MHz, CDCl₃) δ: 0.96 (3H, s), 1.03 (3H, s), 1.25 (3H, t, J=7.4 Hz), 2.03 (3H, s), 2.74 (2H, t, J=7.0 Hz), 2.83 (1H, dd, J=14.0, 6.0 Hz), 3.00 (1H, dd, J=14.4, 7.4 Hz), 3.10 (2H, t, J=7.0 Hz), 3.53 (1H, d, J=13.8 Hz), 3.62 (3H, s), 3.73 (1H, d, J=11.0 Hz), 3.87 (1H, d, J=11.0 Hz), 3.89 (3H, s), 4.16 (2H, q, J=7.4 Hz), 4.37-4.48 (1H, m), 4.57 (1H, d, J=13.8 Hz), 6.31 (1H, s), 6.38 (1H, s), 6.64 (1H, d, J=2.2 Hz), 6.98 (1H, dd, J=7.8, 2.2 Hz), 7.08-7.21 (3H, m), 7.28-7.40 (3H, m), 7.75-7.82 (2H, m).

IR (KBr) 1734, 1678, 1480, 1283, 1242 cm⁻¹.

Elemental Analysis (C₃₉H₄₃N₂O₁₀Cl) Cal'd: C, 63.71; H, 5.90; N, 3.81. Found: C, 63.57; H, 5.70; N, 3.51.

(6) Ethyl 3-[5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-1-benzofuran-2-yl]propanoate (0.24 g, 0.33 mmol) obtained in Example 159-(5) was dissolved in tetrahydrofuran (3 ml) and ethanol (1.5 ml), a 2N aqueous sodium hydroxide solution (0.49 ml) was added at room temperature, and the mixture was stirred at room temperature for 3 hours. The mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (25 ml)-hexane (50 ml), and dried under reduced pressure (50° C.) to obtain 3-[5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-1-benzofuran-2-yl]propanoic acid (0.17 g, yield 78.8%) as white crystals.

m.p. 207.0-209.0° C.

[α]_{D}²²=−123.6° C. (c=0.24, methanol).

¹H-NMR (200 MHz, DMSO-d₆) δ: 0.76 (3H, s), 0.86 (3H, s), 2.65 (2H, t, J=7.6 Hz), 2.83 (2H, d, J=6.2 Hz), 2.98 (2H, t, J=7.6 Hz), 3.03-3.21 (2H, m), 3.51 (3H, s), 3.68 (1H, d, J=13.6 Hz), 3.84 (3H, s), 4.27-4.40 (2H, m), 4.57 (1H, brs), 6.11 (1H, s), 6.39 (1H, d, J=2.2 Hz), 6.57 (1H, s), 7.06-7.18 (3H, s), 7.26 (1H, dd, J=8.4, 1.8 Hz), 7.41 (1H, d, J=8.4 Hz), 7.56 (1H, dd, J=8.4, 2.2 Hz), 7.73 (1H, d, J=8.4 Hz), 7.83 (1H, d, J=1.8 Hz), 10.04 (1H, s).

IR (KBr) 3432, 3400-2500, 1740, 1690, 1651, 1530, 1480 cm⁻¹.

Elemental Analysis (C₃₅H₃₇N₂O₉Cl) Cal'd: C, 63.20; H, 5.61; N, 4.21. Found: C, 63.00; H, 5.60; N, 4.04.

### EXAMPLE 160

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methyl-1-benzofuran-2-carboxylic acid

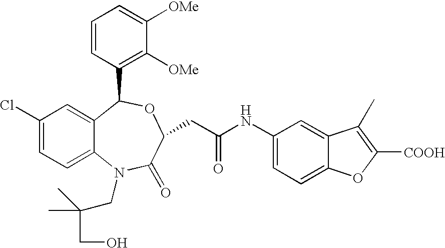

(1) p-Nitrophenol (9.0 g, 64.70 mmol) was dissolved in N,N-dimethylformamide (45 ml), and sodium hydride (60%) (3.1 g, 77.64 mmol) was added under ice-cooling. After stirred at room temperature for 1 hour, methyl 2-chloro-3-oxobutanoate (9.35 ml, 77.64 mmol) was added at room temperature, and the mixture was stirred for 12 hours. 1N Hydrochloric acid was added to the reaction solution, the mixture was extracted with ethyl acetate, and the organic layer was washed with water and an aqueous saturated sodium chloride solution. The organic layer was dried with anhydrous sodium sulfate, and concentrated under reduced pressure. The resulting crude crystals were recrystallized from ethyl acetate (20 ml)-hexane (50 ml) to obtain methyl 2-[(4-nitrophenyl)oxy]-3-oxobutanoate (5.49 g, yield 33.5%) as white crystals.

m.p. 87.5-88.0° C.

¹H-NMR (200 MHz, CDCl₃) δ: 2.00 (3H, s), 3.75 (3H, s), 7.01 (2H, d, J=9.6 Hz), 8.22 (2H, d, J=9.6 Hz).

IR (KBr) 1759, 1734, 1671, 1593, 1508, 1350, 1267 cm⁻¹.

Elemental Analysis (C₁₁H₁₁NO₆) Cal'd: C, 52.18; H, 4.38; N, 5.53. Found: C, 52.25; H, 4.33; N, 5.46.

(2) Methyl 2-[(4-nitrophenyl)oxy]-3-oxobutanoate (1.0 g, 3.95 mmol) obtained in Example 160-(1) was dissolved in concentrated sulfuric acid (5 ml), the solution was stirred at room temperature for 12 hours, and stirred at 40° C. for 4 hours. Allowing to cool, the reaction solution was poured into ice-water, and extracted with ethyl acetate. The organic layer was washed with an aqueous saturated sodium bicarbonate solution, water and an aqueous saturated sodium chloride solution. The organic layer was dried with anhydrous sodium sulfate, and concentrated under reduced pressure. The resulting residue was purified by silica gel column chromatography (hexane:ethyl acetate=3:1) to obtain methyl 3-methyl-5-nitro-1-benzofuran-2-carboxylate (0.48 g, yield 52.0%) as pale yellowish white crystals.

m.p. 156.0-156.5° C.

¹H-NMR (200 MHz, CDCl₃) δ: 2.66 (3H, s), 4.02 (3H, s), 7.65 (1H, d, J=9.2 Hz), 8.38 (1H, dd, J=9.2, 2.2 Hz), 8.61 (1H, d, 2.2 Hz).

IR (KBr) 1730, 1530, 1343 cm⁻¹.

Elemental Analysis (C₁₁N₉NO₅) Cal'd: C, 56.17; H, 3.86; N, 5.96. Found: C, 56.16; H, 3.72; N, 6.03.

(3) Methyl 3-methyl-5-nitro-1-benzofuran-2-carboxylate (0.4 g, 1.70 mmol) obtained in Example 160-(2) was dissolved in ethyl acetate (5 ml), and nitrogen replacement was performed. 10% Palladium carbon (40 mg) was placed therein, and hydrogen was introduced. After stirred at room temperature for 1 hour, the catalyst was filtered, and the filtrate was concentrated under reduced pressure. Ethyl acetate was added to the resulting residue, 4N hydrogen chloride-ethyl acetate (0.43 ml) was added, the mixture was stirred at room temperature for 1 hour, the crystals were filtered, and washed with ethyl acetate. Drying under reduced pressure (50° C.) afforded ethyl 5-amino-3-methyl-1-benzofuran-2-carboxylate hydrochloride (0.39 g, yield 95.1%) as white crystals.

mp253.0-254.0° C.

¹H-NMR (200 MHz, DMSO-d₆) δ: 2.54 (3H, s), 3.91 (3H, s), 7.49 (1H, dd, J=8.8, 2.0 Hz), 7.72 (1H, d, J=2.0 Hz), 7.79 (1H, d, J=8.8 Hz).

IR (KBr) 3300-2300, 1726, 1709, 1595, 1526, 1433 cm⁻¹.

Elemental Analysis (C₁₁H₁₂NO₃Cl) Cal'd: C, 54.67; H, 5.00; N, 5.80. Found: C, 54.53; H, 5.00; N, 5.92.

(4) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.65 g, 1.24 mmol) obtained in Example 1-(1) was dissolved in N,N-dimethylformamide (7 ml) under the argon atmosphere. Triethylamine (0.18 ml, 1.27 mmol) and isobutyl chloroformate (0.19 ml, 1.43 mmol) were added under ice-cooling, the mixture was stirred at the same temperature for 1 hour. Ethyl 5-amino-3-methyl-1-benzofuran-2-carboxylate hydrochloride (0.3 g, 1.24 mmol) obtained in Example 160-(3) was added, and pyridine (0.16 ml, 1.99 mmol) was added dropwise. After stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=2:1) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methyl-1-benzofuran-2-carboxylate (0.84 g, yield 94.6%) as a colorless foam.

[α]_{D}²²=−95.3° (c=0.39, methanol).

¹H-NMR (200 MHz, CDCl₃) δ: 0.97 (3H, s), 1.03 (3H, s), 2.02 (3H, s), 2.55 (3H, s), 2.87 (1H, dd, J=14.2, 6.2 Hz), 3.04 (1H, dd, J=14.2, 7.2 Hz), 3.55 (1H, d, J=13.8 Hz), 3.62 (3H, s), 3.74 (1H, d, J=11.4 Hz), 3.88 (1H, d, J=11.4 Hz), 3.90 (3H, s), 3.98 (3H, s), 4.40-4.50 (1H, m), 4.58 (1H, d, J=13.8 Hz), 6.32 (1H, s), 6.65 (1H, d, J=1.8 Hz), 6.98 (1H, dd, J=7.6, 2.2 Hz), 7.11 (1H, d, J=7.6 Hz), 7.15-7.23 (1H, m), 7.27-7.40 (3H, m), 7.44 (1H, d, J=8.8 Hz), 8.01 (1H, d, J=2.2 Hz), 8.08 (1H, s).

IR (KBr) 3337, 2959, 1721, 1680, 1481 cm⁻¹.

Elemental Analysis (C₃₇H₃₉N₂O₁₀Cl.0.2H₂O) Cal'd: C, 62.52; H, 5.59; N, 3.94. Found: C, 62.53; H, 5.61; N, 4.02.

(5) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-3-methyl-1-benzofuran-2-carboxylate (0.7 g, 0.99 mmol) obtained in Example 160-(4) was dissolved in tetrahydrofuran (7 ml) and ethanol (3.5 ml), a 2N aqueous sodium hydroxide solution (1.48 ml) was added at room temperature, and the mixture was stirred at room temperature for 2 hours. After allowing to cool, the mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (25 ml)-hexane (10 ml), and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-3-methyl-1-benzofuran-2-carboxylic acid (0.49 g, yield 76.6%) as white crystals.

m.p. 175.0-176.5° C.

[α]_{D}²²=−112.3° (c=0.14, methanol).

¹H-NMR (200 MHz, DMSO-d₆): 0.77 (3H, s), 0.86 (3H, s), 2.49 (3H, s), 2.86 (2H, d, J=7.0 Hz), 3.07 (1H, d, J=10.1 Hz), 3.17 (1H, d, J=10.1 Hz), 3.45 (3H, s), 3.68 (1H, d, J=14.2 Hz), 3.84 (3H, s), 4.29-4.40 (2H, m), 4.56 (1H, brs), 6.11 (1H, s), 6.40 (1H, d, J=2.4 Hz), 7.00-7.20 (3H, m), 7.48 (1H, dd, J=9.2, 2.2 Hz), 7.53 -7.62 (2H, m), 7.74 (1H, d, J=9.2 Hz), 8.08 (1H, d, J=2.0 Hz), 10.20 (1H, s).

IR (KBr) 3700-2400, 1705, 1690, 1659, 1480 cm⁻¹.

Elemental Analysis (C₃₄H₃₅N₂O₉Cl.H₂O) Cal'd: C, 61.03; H, 5.57; N, 4.19. Found: C, 61.02; H, 5.39; N, 4.25.

### EXAMPLE 161

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-methyl-1-benzofuran-2-carboxylic acid

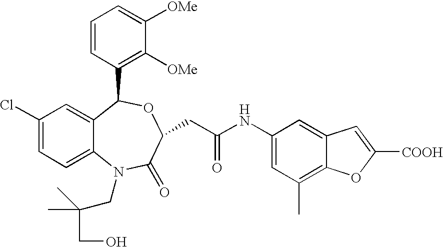

(1) o-Cresol (10 g, 92.47 mmol) was dissolved in acetonitrile (100 ml) under the argon atmosphere, magnesium chloride (13.2 g, 138.71 mmol) was added at room temperature, and triethylamine (48.3 ml, 346.77 mmol) was added dropwise. Subsequently, paraformaldehyde (20 g) was added, and the mixture was stirred under heating at reflux for 2.5 hours. Allowing to cool, the mixture was made acidic using 6N hydrochloric acid, and extracted with ethyl acetate. The organic layer was washed with water and an aqueous saturated sodium chloride solution, and dried with anhydrous sodium sulfate. The organic layer was concentrated under reduced pressure, and the resulting residue was purified by silica gel column chromatography (hexane:ethyl acetate=8:1) to obtain 2-hydroxy-3-methylbenzaldehyde (6.08 g, yield 48.3%) as a pale yellow oil.

¹H-NMR (200 MHz, CDCl₃) δ: 2.27 (3H, s), 6.93 (1H, t, J=7.4 Hz), 7.40 (2H, d, J=7.4 Hz), 9.88 (1H, s), 11.27 (1H, s).

IR (KBr) 3500-2600, 1661, 1644 cm⁻¹.

(2) Fuming nitric acid (d=1.52) (10 ml) was ice-cooled, 2-hydroxy-3-methylbenzaldehyde (5.5 g, 40.40 mmol) obtained in Example 161-(1) was gradually added dropwise, and the mixture was stirred at the same temperature for 1 hour. The reaction solution was poured into ice-water, and extracted with ethyl acetate. The organic layer was washed with an aqueous saturated sodium bicarbonate solution, water and an aqueous saturated sodium chloride solution, dried with anhydrous sodium sulfate, and concentrated under reduced pressure. The resulting crude crystals were purified by silica gel column chromatography (hexane:ethyl acetate=4:1) to obtain 2-hydroxy-3-methyl-5-nitrobenzaldehyde (2.25 g, yield 30.7%) as pale yellow crystals.

m.p. 131.5-133.0° C.

¹H-NMR (200 MHz, CDCl₃): 2.37 (3H, s), 8.30 (1H, d, J=2.4 Hz), 8.42 (1H, d, J=2.4 Hz), 9.88 (1H, s), 11.89 (1H, s).

IR (KBr) 3400-2700, 1653, 1624, 1516, 1352 cm⁻¹.

Elemental Analysis (C₈H₇NO₄) Cal'd: C, 53.04; H, 3.89; N, 7.73. Found: C, 53.19; H, 3.65; N, 7.75.

(3) 2-Hydroxy-3-methyl-5-nitrobenzaldehyde (1.0 g, 5.52 mmol) obtained in Example 161-(2) was dissolved in N,N-dimethylformamide (10 ml), and potassium carbonate (1.91 g, 13.80 mmol) was added. Ethyl bromoacetate (0.73 ml, 6.62 mmol) was added at room temperature, the mixture was stirred for 1 hour, and stirred at 80° C. for 17 hours. After allowing to cool, water was added, and the mixture was extracted with ethyl acetate. The organic layers were combined, and washed with water and an aqueous saturated sodium chloride solution. The mixture was dried with anhydrous sodium sulfate, and concentrated under reduced pressure. The resulting crude crystals were purified by silica gel column chromatography (hexane:ethyl acetate=6:1) to obtain ethyl 7-methyl-5-nitro-1-benzofuran-2-carboxylate (0.21 g, yield 15.0%) as pale yellowish white crystals.

mp124.9-125.5° C.

¹H-NMR (200 MHz, CDCl₃): 1.45 (3H, t, J=7.4 Hz), 2.67 (3H, s), 4.48 (2H, q, J=7.4 Hz), 7.62 (1H, s), 8.17 (1H, d, J=2.6 Hz), 8.46 (1H, d, J=2.6 Hz).

IR (KBr) 1732, 1717, 1526, 1348, 1296 cm⁻¹.

Elemental Analysis (C₂₁H₁₁NO₅) Cal'd: C, 57.83; H, 4.45; N, 5.62. Found: C, 57.74; H, 4.29; N, 5.63.

(4) Ethyl 7-methyl-5-nitro-1-benzofuran-2-carboxylate (0.4 g, 1.61 mmol) obtained in Example 161-(3) was dissolved in ethyl acetate (8 ml), and nitrogen replacement was performed. 10% palladium carbon (40 mg) was placed therein, and hydrogen was introduced. The mixture was stirred at room temperature for 2 hours, the catalyst was filtered, and the filtrate was concentrated under reduced pressure. Ethyl acetate was added to the resulting residue, 4N hydrogen chloride-ethyl acetate (0.4 ml) was added, the mixture was stirred at room temperature for 1 hour, the crystals were filtered, and washed with ethyl acetate. Drying under reduced pressure (50° C.) afforded ethyl 5-amino-7-methyl-1-benzofuran-2-carboxylate hydrochloride (0.38 g, yield 91.4%) as white crystals.

m.p. 256.0-258.0° C.

¹H-NMR (200 MHz, DMSO-d₆) δ: 1.34 (3H, t, J=7.4 Hz), 2.54 (3H, s), 4.38 (2H, q, J=7.4 Hz), 7.30 (1H, d, J=1.8 Hz), 7.63 (1H, d, J=1.8 Hz), 7.84 (1H, s).

IR (KBr) 3200-2300, 1742, 1550 cm⁻¹.

Elemental Analysis (C₁₂H₁₄NO₃Cl) Cal'd: C, 56.37; H, 5.52; N, 5.48. Found: C, 56.19; H, 5.51; N, 5.59.

(5) (3R,5S)-1-(3-acetoxy-2,2-dimethylpropyl)-7-chloro-5-(2,3-dimethoxyphenyl)-1,2,3,5-tetrahydro-2-oxo-4,1-benzoxazepine-3-acetic acid (0.61 g, 1.17 mmol) obtained in Example 1-(1) was dissolved in N,N-dimethylformamide (6 ml) under the argon atmosphere. Triethylamine (0.17 ml, 1.20 mmol) and isobutyl chloroformate (0.18 ml, 1.139 mmol) were added under ice-cooling, the mixture was stirred at the same temperature for 1 hour. Ethyl 5-amino-7-methyl-1-benzofuran-2-carboxylate hydrochloride (0.3 g, 1.17 mmol) obtained in Example 161-(4) was added, and pyridine (0.15 ml, 1.88 mmol) was added dropwise. After stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=1:l) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-methyl-1-benzofuran-2-carboxylate (0.81 g, yield 95.5%) as a colorless foam.

[α]_{D}²²=−101.1° (c=0.31, methanol).

¹H-NMR (200 MHz, CDCl₃) δ: 0.97 (3H, s), 1.03 (3H, s), 1.43 (3H, t, J=7.0 Hz), 2.03 (3H, s), 2.55 (3H, s), 2.85 (1H, dd, J=14.2, 6.2 Hz), 3.01 (1H, dd, J=14.2, 7.0 Hz), 3.54 (1H, d, J=14.0 Hz), 3.62 (3H, s), 3.74 (1H, d, J=11.0 Hz), 3.88 (1H, d, J=11.0 Hz), 3.90 (3H, s), 4.38-4.50 (2H, m), 4.57 (1H, d, J=14.0 Hz), 6.32 (1H, s), 6.65 (1H, d, J=2.2 Hz), 6.99 (1H, dd, J=7.8, 2.2 Hz), 7.11 (1H, d, J=7.6 Hz), 7.15-7.25 (2H, m), 7.30-7.40 (2H, m), 7.45 (1H, s), 7.83 (1H, s), 7.91 (1H, s).

IR (KBr) 3335, 2967, 1732, 1680, 1481, 1287 cm⁻¹.

Elemental Analysis (C₃₈H₄₁N₂O₁₀Cl) Cal'd: C, 63.29; H, 5.73; N, 3.88. Found: C, 63.20; H, 5.66; N, 3.76.

(6) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-methyl-1-benzofuran-2-carboxylate (0.7 g, 0.97 mmol) obtained in Example 161-(5) was dissolved in tetrahydrofuran (3.5 ml) and ethanol (3.5 ml), a 2N aqueous sodium hydroxide solution (1.46 ml) was added at room temperature, and the mixture was stirred at room temperature for 1.5 hours. After allowing to cool, the mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (25 ml)-hexane (10 ml), and dried under reduced pressure (50° C.) to obtain 5-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-methyl-1-benzofuran-2-carboxylic acid (0.49 g, yield 77.2%) as white crystals.

m.p. 180.7-182.0° C.

[α]_{D}²²=−120.9° (c=0.18, methanol).

¹H-NMR (200 MHz, DMSO-d₆): 0.77 (3H, s), 0.86 (3H, s), 2.46 (3H, s), 2.85 (2H, d, J=6.2 Hz), 3.07 (1H, d, J=10.2 Hz), 3.17 (1H, d, J=10.2 Hz), 3.52 (3H, s), 3.68 (1H, d, J=14.8 Hz), 3.84 (3H, s), 4.27-4.39 (2H, m), 4.56 (1H, brs), 6.12 (1H, s), 6.40 (1H, d, J=2.2 Hz), 7.65 (1H, s), 7.74 (1H, d, J=8.8 Hz), 7.91 (1H, d, J=2.4 Hz), 10.15 (1H, s).

IR (KBr) 3700-2300, 1726, 1692, 1655, 1545, 1480 cm⁻¹.

Elemental Analysis (C₃₄H₃₅N₂O₉Cl) Cal'd: C, 62.72; H, 5.42; N, 4.30. Found: C, 62.77; H, 5.67; N, 4.02.

### EXAMPLE 162

5-[[2-[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-ethyl-1-benzofuran-2-carboxylic acid

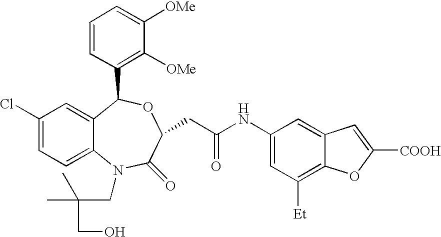

(1) 2-Ethylphenol (10 g, 81.85 mmol) was dissolved in acetonitrile (100 ml) under the argon atmosphere, magnesium chloride (11.7 g, 122.78 mmol) was added at room temperature, and triethylamine (42.8 ml, 306.95 mmol) was added dropwise. Subsequently, paraformaldehyde (9.49 g) was added, and the mixture was stirred under heating at reflux for 3 hours. Allowing to cool, the mixture was made acidic using 6N hydrochloric acid, the insolubles were filtered using Celite, and extracted with diethyl ether. The organic layer was washed with water and an aqueous saturated sodium chloride solution, and dried with anhydrous sodium sulfate. The organic layer was concentrated under reduced pressure to obtain a brown oil. Fuming nitric acid (d=1.52) (3.39 ml, 81.85 mmol) was added dropwise to ice-cooled acetic anhydride (20 ml), and the previously obtained brown oil was gradually added dropwise. The mixture was stirred at the same temperature for 2 hours, an aqueous saturated sodium bicarbonate solution was added, and the mixture was extracted with ethyl acetate. The organic layer was washed with water and an aqueous saturated sodium chloride solution, dried with anhydrous sodium sulfate, and concentrated under reduced pressure. The resulting crude crystals were recrystallized from methanol to obtain 3-ethyl-2-hydroxy-5-nitrobenzaldehyde (6.21 g, yield 38.9%) as a pale yellow crystal.

m.p. 93.5-94.0° C.

¹H-NMR (200 MHz, CDCl₃): 1.29 (3H, t, J=7.8 Hz), 2.78 (2H, q, J=7.8 Hz), 8.30 (1H, d, J=2.6 Hz), 8.42 (1H, d, J=2.6 Hz), 9.99 (1H, s), 11.92 (1H, s).

IR (KBr) 3400-2700, 1674, 1618, 1518, 1451, 1360 cm¹.

Elemental Analysis (C₉H₉NO₄) Cal'd: C, 55.39; H, 4.65; N, 7.18. Found: C, 55.28; H, 4.44; N, 7.26.

(2) 3-Ethyl-2-hydroxy-5-nitrobenzaldehyde (3.0 g, 15.37 mmol) obtained in Example 162-(1) was dissolved in N,N-dimethylformamide (30 ml), and potassium carbonate (4.25 g, 30.74 mmol) was added. Ethyl bromoacetate (1.97 ml, 18.45 mmol) was added at room temperature, the mixture was stirred for 1 hour, and stirred at 80° C. for 12 hours. After allowing to cool, water was added, and the mixture was extracted with ethyl acetate. The organic layers were combined, and washed with water and an aqueous saturated sodium chloride solution. The mixture was dried with anhydrous sodium sulfate, and concentrated under reduced pressure. The resulting crude crystals were recrystallized from methanol to obtain ethyl 7-ethyl-5-nitro-1-benzofuran-2-carboxylate (1.73 g, yield 42.8%) as a pale yellowish white crystal.

m.p. 114.5-115.5° C.

¹H-NMR (200 MHz, CDCl₃): 1.42 (3H, t, J=7.2 Hz), 1.44 (3H, t, J=7.2 Hz), 3.08 (2H, q, J=7.2 Hz), 4.47 (2H, q, J=7.2 Hz), 7.62 (1H, s), 8.20 (1H, d, J=2.2 Hz), 8.47 (1H, d, J=2.2 Hz).

IR (KBr) 1732, 1532, 1348, 1188 cm⁻¹.

Elemental Analysis (C₁₃H₁₃NO₆) Cal'd: C, 59.31; H, 4.98; N, 5.32. Found: C, 59.31; H, 4.92; N, 5.35.

(3) Ethyl 7-ethyl-5-nitro-1-benzofuran-2-carboxylate (1.0 g, 3.80 mmol) obtained in Example 162-(2) was dissolved in ethyl acetate (10 ml), and nitrogen replacement was performed. 10% palladium carbon (100 mg) was placed therein, and hydrogen was introduced. The mixture was stirred at room temperature for 2 hours, the catalyst was filtered, and the filtrate was concentrated under reduced pressure. Ethyl acetate was added to the resulting residue, 4N hydrogen chloride-ethyl acetate (0.95 ml) was added, the mixture was stirred at room temperature for 1 hour, the crystals were filtered, and washed with ethyl acetate. Drying under reduced pressure (50° C.) afforded ethyl 5-amino-7-ethyl-1-benzofuran-2-carboxylate hydrochloride (0.93 g, yield 90.8%) as white crystals.

m.p. 242.5-243.0° C.

¹H-NMR (200 MHz, DMSO-d₆) δ: 1.30 (3H, t, J=7.8 Hz), 1.34 (3H, t, J=7.0 Hz), 2.94 (2H, q, J=7.8 Hz), 4.38 (2H, q, J=7.0 Hz), 7.29 (1H, d, J=2.2 Hz), 7.59 (1H, d, J=2.2 Hz), 7.83 (1H, s).

IR (KBr) 3200-2300, 1717, 1580, 1308 cm⁻¹.

Elemental Analysis (C₁₃H₁₆NO₃Cl) Cal'd: C, 57.89; H, 5.98; N, 5.19. Found: C, 58.04; H, 5.97; N, 5.25.

(4) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.27 ml, 2.21 mmol) were added under ice-cooling, the mixture was stirred at the same temperature for 1 hour. Ethyl 5-amino-7-ethyl-1-benzofuran-2-carboxylate hydrochloride (0.52 g, 1.92 mmol) obtained in Example 162-(3) was added, and pyridine (0.25 ml, 3.08 mmol) was added dropwise. After stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=3:2) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-ethyl-1-benzofuran-2-carboxylate (1.32 g, yield 93.4%) as a colorless foam.

[α]_{D}²²=−92.3° (c=0.25, methanol).

¹H-NMR (200 MHz, CDCl₃) δ: 0.96 (3H, s), 1.03 (3H, s), 1.34 (3H, t, J=7.8 Hz), 1.42 (3H, t, J=7.2 Hz), 2.02 (3H, s), 2.85 (1H, dd, J=14.2, 5.8 Hz), 2.96 (2H, q, J=7.8 Hz), 3.02 (1H, dd, J=14.2, 7.4 Hz), 3.54 (1H, d, J=14.2 Hz), 3.62 (3H, s), 3.74 (1H, d, J=11.0 Hz), 3.88 (1H, d, J=11.0 Hz), 3.90 (3H, s), 4.34-4.90 (3H, m), 4.57 (1H, d, J=14.2 Hz), 6.32 (1H, s), 6.65 (1H, d, J=2.0 Hz), 6.99 (1H, dd, J=7.4, 1.8 Hz), 7.11 (1H, d, J=7.8 Hz), 7.14-7.22 (2H, m), 7.30-7.40 (2H, m), 7.44 (1H, s), 7.86 (1H, d, J=2.2 Hz), 7.96 (1H, s).

IR (KBr) 2971, 1732, 1680, 1481 cm⁻¹.

Elemental Analysis (C₃₉H₄₃N₂O₁₀Cl) Cal'd: C, 63.71; H, 5.90; N, 3.81. Found: C, 63.42; H, 5.86; N, 3.75.

(5) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-ethyl-1-benzofuran-2-carboxylate (1.0 g, 1.36 mmol) obtained in Example 162-(4) was suspended in ethanol (20 ml), a 2N aqueous sodium hydroxide solution (2 ml) was added at room temperature, and the mixture was stirred at room temperature for 2 hours. After allowing to cool, 1N hydrochloric acid (4 ml) was added, water (12 ml) was added, and the mixture was stirred at room temperature for 3 hours. The crystals were filtered off, washed with water, dried under reduced pressure (50° C.) to obtain 5-[[[-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-ethyl-1-benzofuran-2-carboxylic acid (0.85 g, yield 93.4%) as white crystals.

m.p. 188.0-189.0° C.

[α]_{D}²²=−116.9° (c=0.13, methanol).

¹H-NMR (200 MHz, DMSO-d₆) δ: 0.77 (3H, s), 0.86 (3H, s), 1.28 (3H, t, J=7.6 Hz), 2.78-2.91 (4H, m), 3.07 (1H, d, J=10.2 Hz), 3.17 (1H, d, J=10.2 Hz), 3.52 (3H, s), 3.68 (1H, d, J=13.8 Hz), 3.84 (3H, s), 4.27-4.40 (2H, m), 4.56 (1H, brs), 6.11 (1H, s), 6.40 (1H, d, J=2.2 Hz), 7.05-7.19 (3H, m), 7.36 (1H, d, J=2.0 Hz), 7.56 (1H, dd, J=8.8, 2.2 Hz), 7.63 (1H, s), 7.74 (1H, d, J=8.8 Hz), 7.94 (1H, d, J=2.0 Hz), 10.13 (1H, s).

IR (KBr) 3700-2200, 1725, 1694, 1663, 1545, 1478 cm⁻¹.

Elemental Analysis (C₃₅H₃₇N₂O₉Cl.H₂O) Cal'd: C, 61.54; H, 5.75; N, 4.10. Found: C, 61.53; H, 5.80; N, 4.08.

### EXAMPLE 163

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-propyl-1-benzofuran-2-carboxylic acid

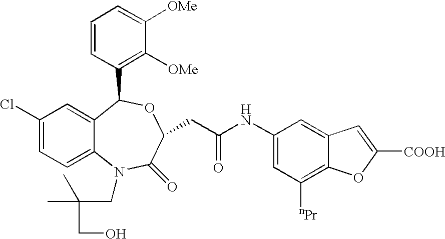

(1) 2-Propylphenol (10 g, 73.43 mmol) was dissolved in acetonitrile (100 ml) under the argon atmosphere, magnesium chloride (10.5 g, 110.14 mmol) was added at room temperature, and triethylamine (38.4 ml, 275.35 mmol) was added dropwise. Subsequently, paraformaldehyde (8.5 g) was added, and the mixture was stirred under heating at reflux for 1.5 hours. Allowing to cool, the mixture was made acidic using 6N hydrochloric acid, the insolubles were filtered using Celite. The filtrate was extracted with ethyl acetate, the organic layer was washed with water and an aqueous saturated sodium chloride solution, and dried with anhydrous sodium sulfate. The organic layer was concentrated under reduced pressure, the resulting residue was purified by silica gel column chromatography (hexane:ethyl acetate=9:1) to obtain 2-hydroxy-3-propylbenzaldehyde (10.13 g, yield 84.0%) as a yellow oil.

¹H-NMR (200 MHz, CDCl₃) δ: 0.96 (3H, t, J=7.4 Hz), 1.56-1.78 (2H, m), 2.65 (2H, t, J=7.4 Hz), 6.95 (1H, t, J=7.2 Hz), 7.34-7.45 (2H, m), 9.88 (1H, s), 11.27 (1H, s).

IR (KBr) 3700-2600, 1653, 1617, 1447 cm⁻¹.

(2) Fuming nitric acid (d=1.52) (2.30 ml, 55.42 mmol) was added dropwise to ice-cooled acetic anhydride (21 ml), and 2-hydroxy-3-propylbenzaldehyde (7.0 g, 42.63 mmol) obtained in Example 163-(1) was gradually added dropwise. The mixture was stirred at the same temperature for 2 hours, an aqueous saturated sodium bicarbonate solution was added, and the mixture was extracted with ethyl acetate. The organic layer was washed with water and an aqueous saturated sodium chloride solution, dried with anhydrous sodium sulfate, and concentrated under reduced pressure. The resulting crude crystals were purified by silica gel column chromatography (hexane:ethyl acetate=8:1-12:1) to obtain 2-hydroxy-5-nitro-3-propylbenzaldehyde (5.9 g, yield 66.2%) as pale yellow crystals.

m.p. 69.5-70.0° C.

¹H-NMR (200 MHz, CDCl₃) δ: 1.00 (3H, t, J=7.2 Hz), 1.60-1.80 (2H, m), 2.73 (2H, q, J=7.2 Hz), 8.28 (1H, d, J=3.0 Hz), 8.43 (1H, d, J=3.0 Hz), 9.99 (1H, s), 11.91 (1H, s).

IR (KBr) 3400-2400, 1661, 1624, 1537, 1447, 1345 cm⁻¹

Elemental Analysis (C₁₀H₁₁NO₄) Cal'd: C, 57.41; H, 5.30; N, 6.70. Found: C: 57.46, H: 5.31, N: 6.78.

(3) 2-Hydroxy-5-nitro-3-propylbenzaldehyde (5.9 g, 28.20 mmol) obtained in Example 163-(2) was dissolved in N,N-dimethylformamide (60 ml), and potassium carbonate (7.80 g, 56.41 mmol) was added. Ethyl bromoacetate (3.75 ml, 33.84 mmol) was added at room temperature, the mixture was stirred for 1 hour, and stirred at 80° C. for 12 hours. After allowing to cool, water was added, and the mixture was extracted with ethyl acetate. The organic layers were combined, and washed with water and an aqueous saturated sodium chloride solution. The mixture was dried with anhydrous sodium sulfate, and concentrated under reduced pressure. The resulting crude crystals were recrystallized from methanol to obtain ethyl 5-nitro-7-propyl-1-benzofuran-2-carboxylate (2.84 g, yield 36.3%) as a pale yellowish white crystal.

m.p. 110.6-111.0° C.

¹H-NMR (200 MHz, CDCl₃) δ: 1.03 (3H, t, J=7.2 Hz), 1.45 (3H, t, J=7.4 Hz), 1.84 (2H, m), 3.02 (2H, t, J=7.4 Hz), 4.47 (2H, q, J=7.2 Hz), 7.62 (1H, s), 8.18 (1H, d, J=2.2 Hz), 8.47 (1H, d, J=2.2 Hz).

IR (KBr) 1738, 1530, 1343, 1196 cm⁻¹.

Elemental Analysis (C₁₄H₁₁NO₅) Cal'd: C, 60.64; H, 5.45; N, 5.05. Found: C, 60.57; H, 5.38; N, 5.09.

(4) Ethyl 5-nitro-7-propyl-1-benzofuran-2-carboxylate (1.5 g, 5.41 mmol) obtained in Example 163-(3) was dissolved in ethyl acetate (15 ml), and nitrogen replacement was performed. 10% palladium carbon (150 mg) was placed therein, and hydrogen was introduced. The mixture was stirred at room temperature for 2 hours, the catalyst was filtered, and the filtrate was concentrated under reduced pressure. Ethyl acetate was added to the resulting residue, 4N hydrogen chloride-ethyl acetate (1.35 ml) was added, the mixture was stirred at room temperature for 1 hour, the crystals were filtered, and washed with ethyl acetate. Drying under reduced pressure (50° C.) afforded methyl 5-amino-7-propyl-1-benzofuran-2-carboxylate hydrochloride (1.5 g, yield 97.7%) as white crystals.

m.p. 200.5-201.5° C.

¹H-NMR (200 MHz, DMSO-d₆) δ: 0.95 (3H, t, J=7.0 Hz), 1.34 (3H, t, J=7.0 Hz), 1.73 (2H, m), 2.89 (2H, t, J=7.0 Hz), 4.38 (2H, q, J=7.0 Hz), 7.31 (1H, d, J=2.2 Hz), 7.64 (1H, d, J=2.2 Hz), 7.84 (1H, s).

IR (KBr) 3300-2400, 1719, 1574, 1306 cm⁻¹.

Elemental Analysis (C₁₄H₁₈NO₃Cl) Cal'd: C, 59.26; H, 6.39; N, 4.49. Found: C, 59.23; H, 6.27; N, 4.92.

(5) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.27 ml, 2.21 mmol) were added under ice-cooling, the mixture was stirred at the same temperature for 1 hour. Ethyl 5-amino-7-propyl-1-benzofuran-2-carboxylate hydrochloride (0.55 g, 1.92 mmol) obtained in Example 163-(4) was added, and pyridine (0.25 ml, 3.08 mmol) was added dropwise. After stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=2:1) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-propyl-1-benzofuran-2-carboxylate (1.33 g, yield 92.3%) as a colorless foam.

[α]_{D}²²=−98.3° (c=0.25, methanol).

¹H-NMR (200 MHz, CDCl₃) δ: 0.96 (3H, s), 0.99 (3H, t, J=7.0 Hz), 1.03 (3H, s), 1.42 (3H, t, J=7.4 Hz), 1.77 (2H, m), 2.02 (3H, s), 2.80-2.95 (3H, m), 3.02 (1H, dd, J=14.2, 7.2 Hz), 3.63 (1H, d, J=14.0 Hz), 3.62 (3H, s), 3.74 (1H, d, J=11.4 Hz), 3.90 (3H, s), 4.36-4.50 (3H, m), 4.57 (1H, d, J=14.0 Hz), 6.31 (1H, s), 6.65 (1H, d, J=1.8 Hz), 6.98 (1H, dd, J=7.8, 1.8 Hz), 7.11 (1H, d, J=7.8 Hz), 7.16-7.23 (2H, m), 7.30-7.40 (2H, m), 7.44 (1H, s), 7.86 (1H, s, J=2.2 Hz), 7.95 (1H, s).

IR (KBr) 3335, 2967, 1732, 1680, 1481, 1287 cm⁻¹.

Elemental Analysis (C₄₀H₄₅N₂O₁₀Cl) Cal'd: C, 64.12; H, 6.05; N, 3.74. Found: C, 63.95; H, 6.06; N, 3.69.

(6) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-7-propyl-1-benzofuran-2-carboxylate (1.0 g, 1.34 mmol) obtained in Example 163-(5) was dissolved in tetrahydrofuran (4 ml) and ethanol (4 ml), a 2N aqueous sodium hydroxide solution (2 ml) was added at room temperature, and the mixture was stirred at room temperature for 1.5 hours. After allowing to cool, the mixture was neutralized using 1N hydrochloric acid, concentrated under reduced pressure, ethyl acetate and water were added, and the layers were separated. The organic layer was washed with water and an aqueous saturated sodium chloride solution, dried with anhydrous sodium sulfate, and concentrated under reduced pressure. The resulting crude crystals were recrystallized from ethyl acetate (60 ml)-hexane (30 ml), and dried under reduced pressure (50° C.) to obtain 5-[[[-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-7-propyl-1-benzofuran-2-carboxylic acid (0.79 g, yield 87.5%) as white crystals.

m.p. 198.5-199.5° C.

[α]_{D}²²=−97.5° (c=0.28, methanol).

¹H-NMR (200 MHz, DMSO-d₆): 0.77 (3H, s), 0.86 (3H, s), 0.94 (3H, t, J=7.4 Hz), 1.71 (2H, m), 2.70-2.90 (4H, m), 3.00-3.20 (2H, m), 3.52 (3H, s), 3.68 (1H, d, J=14.0 Hz), 3.84 (3H, s), 4.27-4.40 (2H, m), 4.55 (1H, brs), 6.11 (1H, s), 6.40 (1H, d, J=2.6 Hz), 7.05-7.20 (3H, m), 7.35 (1H, d, J=1.8 Hz), 7.56 (1H, dd, J=8.8, 2.6 Hz), 7.63 (1H, s), 7.74 (1H, d, J=8.8 Hz), 7.94 (1H, d, J=21.8 Hz), 10.12 (1H, s).

IR (KBr) 3600-2500, 1728, 1686, 1624, 1570, 1483 cm⁻¹.

Elemental Analysis (C₃₆H₃₉N₂O₉Cl.0.5H₂O) Cal'd: C, 62.83; H, 5.86; N, 4.07. Found: C, 62.96; H, 5.96; N, 4.03.

### EXAMPLE 164

5-[[[(3R,5S)-7-Chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4,6,7-trimethyl-1-benzofuran-2-carboxylic acid

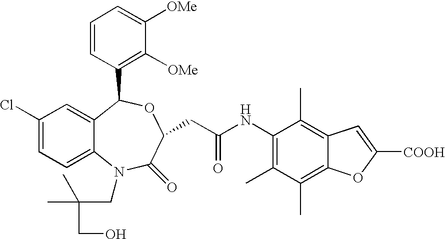

(1) 1,2,4-Trimethylphenol (10.0 g, 73.43 mmol) was dissolved in acetonitrile (100 ml) under the argon atmosphere, magnesium chloride (10.5 g, 110.14 mmol) was added at room temperature, and triethylamine (38.4 ml, 275.35 mmol) was added dropwise. Subsequently, paraformaldehyde (7.5 g) was added, and the mixture was stirred under heating at reflux for 2 hours. Allowing to cool, the mixture was made acidic using 6N hydrochloric acid, the insolubles were filtered using Celite. The filtrate was extracted with ethyl acetate, the organic layer was washed with water and an aqueous saturated sodium chloride solution, and dried with anhydrous sodium sulfate. The organic layer was concentrated under reduced pressure, the resulting residue was purified by silica gel column chromatography (hexane:ethyl acetate=20:1) to obtain 2-hydroxy-3,4,6-trimethylbenzaldehyde (8.78 g, yield 72.8%) as a yellow crystal.

m.p. 74.0-75.5° C.

¹H-NMR (200 MHz, CDCl₃) δ: 2.13 (3H, s), 2.27 (3H, s), 2.53 (3H, s), 6.53 (1H, s), 10.23 (1H, s), 12.30 (1H, s).

IR (KBr) 3400-2500, 1634, 1400, 1350, 1306, 1242 cm⁻¹.

Elemental Analysis (C₁₀H₁₂O₂) Cal'd: C, 73.15; H, 7.37. Found: C, 73.22; H, 7.26.

(2) Fuming nitric acid (d=1.52) (2.12 ml, 51.16 mmol) was added dropwise to ice-cooled acetic anhyride (21 ml), and 2-hydroxy-3,4,6-trimethylbenzaldehyde (7.0 g, 42.63 mmol) obtained in Example 164-(1) was gradually added. The mixture was stirred at the same temperature for 2 hours, an aqueous saturated sodium bicarbonate solution was added, and the mixture was extracted with ethyl acetate. The organic layer was washed with water and an aqueous saturated sodium chloride solution, dried with anhydrous sodium sulfate, and concentrated under reduced pressure. The resulting crude crystals were purified by silica gel column chromatography (hexane: ethyl acetate=10:1) to obtain 2-hydroxy-5-nitro-3,4,6-trimethylbenzaldehyde (3.18 g, yield 35.7%) as pale yellow crystals.

m.p. 161.5-163.0° C.

¹H-NMR (200 MHz, CDCl₃) δ: 2.21 (3H, s), 2.24 (3H, s), 2.49 (3H, s), 10.29 (1H, s).

IR (KBr) 1645, 1526, 1372, 1298 cm⁻¹.

Elemental Analysis (C₁₀H₁₁NO₄) Cal'd: C, 57.41; H, 5.30; N, 6.70. Found: C, 57.63; H, 5.31; N, 6.67.

(3) 2-Hydroxy-5-nitro-3,4,6-trimethylbenzaldehyde (3.18 g, 15.20 mmol) obtained in Example 164-(2) was dissolved in N,N-dimethylformamide (32 ml), and potassium carbonate (4.2 g, 30.40 mmol) was added. Ethyl bromoacetate (2.02 ml, 18.24 mmol) was added at room temperature, the mixture was stirred for 1 hour, and stirred at 75° C. for 12 hours. After allowing to cool, water was added, and the mixture was extracted with ethyl acetate. The organic layers were combined, and washed with water and an aqueous saturated sodium chloride solution. The mixture was dried with anhydrous sodium sulfate, and concentrated under reduced pressure. The resulting crude crystals were recrystallized from methanol to obtain ethyl 5-nitro-4,6,7-trimethyl-1-benzofuran-2-carboxylate (2.55 g, yield 60.5%) as a pale yellowish white crystal.

m.p. 126.5-127.5° C.

¹H-NMR (200 MHz, CDCl₃) δ: 1.44 (3H, t, J=7.0 Hz), 2.31 (3H, s), 2.45 (3H, s), 2.52 (3H, s), 4.45 (2H, q, J=7.0 Hz), 7.56 (1H, s).

IR (KBr) 1732, 1530, 1200 cm⁻¹.

Elemental Analysis (C₁₄H₁₅NO₅) Cal'd: C, 60.64; H, 5.45; N, 5.05. Found: C, 60.50; H, 5.37; N, 5.04.

(4) Ethyl 5-nitro-4,6,7-trimethyl-1-benzofuran-2-carboxylate (1.55 g, 5.59 mmol) obtained in Example 164-(3) was dissolved in ethyl acetate (25 ml), and nitrogen replacement was performed. 10% palladium carbon (300 mg) was placed therein, and hydrogen was introduced. The mixture was stirred at 45° C. for 39 hours, the catalyst was filtered, and the filtrate was concentrated under reduced pressure. The resulting residue was purified by silica gel column chromatography (hexane:ethyl acetate=2:1). The resulting crystals (0.92 g) were dissolved in ethyl acetate, 4N hydrogen chloride-ethyl acetate was added, the mixture was stirred at room temperature for 1 hour, the crystals were filtered off, and washed with ethyl acetate. Drying under reduced pressure (50° C.) afforded ethyl 5-amino-4,6,7-trimethyl-1-benzofuran-2-carboxylate hydrochloride (0.86 g, yield 54.1%) as white crystals.

m.p. 265.0-268.0° C.

¹H-NMR (200 MHz, DMSO-d₆) δ: 1.34 (3H, d, J=7.0 Hz), 2.38 (3H, s), 2.43 (3H, s), 2.55 (3H, s), 4.37 (2H, q, J=7.0 Hz), 7.90 (1H, s).

IR (KBr) 3200-2300, 1716, 1570, 1321, 1277, 1208 cm⁻¹.

Elemental Analysis (C₁₄H₁₈NO₃Cl) Cal'd: C, 59.26; H, 6.39; N, 4.94. Found: C, 59.29; H, 6.32; N, 5.00.

(5) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (1.0 g, 1.92 mmol) obtained in Example 1-(1) was dissolved in N,N-dimethylformamide (10 ml) under the argon atmosphere. Triethylamine (0.27 ml, 1.96 mmol) and isobutyl chloroformate (0.27 ml, 2.21 mmol) were added under ice-cooling, the mixture was stirred at the same temperature for 1 hour. Ethyl 5-amino-4,6,7-trimethyl-1-benzofuran-2-carboxylate hydrochloride (0.55 g, 1.92 mmol) obtained in Example 164-(4) was added, and pyridine (0.25 ml, 3.08 mmol) was added dropwise. After stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=1:1) to obtain ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4,6,7-trimethyl-1-benzofuran-2-carboxylate (1.21 g, yield 84.0%) as a colorless foam.

[α]_{D}²²=−116.2° (c=0.18 methanol).

¹H-NMR (200 MHz, CDCl₃) δ: 0.97 (3H, s), 1.04 (3H, s), 1.43 (3H, t, J=7.2 Hz), 2.04 (3H, s), 2.22 (3H, s), 2.34 (3H, s), 2.46 (3H, s), 2.90 (1H, dd, J=14.2, 4.8 Hz), 3.16 (1H, dd, J=14.2, 7.8 Hz), 3.56 (1H, d, J=13.8 Hz), 3.63 (3H, s), 3.73 (1H, d, J=11.0 Hz), 3.88 (1H, d, J=11.0 Hz), 3.90 (3H, s), 4.35-4.62 (4H, m), 6.32 (1H, s), 6.67 (1H, d, J=2.2 Hz), 7.00 (1H, dd, J=7.6, 2.2 Hz), 7.10-7.24 (2H, m), 7.30-7.39 (2H, m), 7.48-7.53 (2H, m).

IR (KBr) 3227, 2965, 1732, 1678, 1481 cm¹.

Elemental Analysis (C₄₀H₄₅N₂O₁₀Cl) Cal'd: C, 64.12; H, 6.05; N, 3.74. Found: C, 63.88; H, 6.07; N, 3.82.

(6) Ethyl 5-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4,6,7-trimethyl-1-benzofuran-2-carboxylate (0.9 g, 1.20 mmol) obtained in Example 164-(5) was suspended in tetrahydrofuran (4.5 ml) and ethanol (4.5 ml), a 2N aqueous sodium hydroxide solution (1.8 ml) was added at room temperature, and the mixture was stirred at room temperature for 1.5 hours. After allowing to cool, 1N hyddrochloric acid (3.6 ml) was added, water (5.4 ml) was added, and the mixture was stirred at room temperature for 2 hours. The crystals were filtered off, washed with water, and dried under reduced pressure (50° C.) to obtain 5-[[2-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4,6,7-trimethyl-1-benzofuran-2-carboxylic acid (0.72 g, yield 87.6%) as white crystals.

m.p. 246.0-248.0° C.

[α]_{D}²²=−127.5° (c=0.30, methanol).

¹H-NMR (200 MHz, DMSO-d₆) δ: 0.78 (3H, s), 0.87 (3H, s), 2.15 (3H, s), 2.28 (3H, s), 2.39 (3H, s), 2.83-2.93 (2H, m), 3.00-3.21 (2H, m), 3.53 (3H, s), 3.67 (1H, d, J=14.2 Hz), 3.85 (3H, s), 4.28-4.42 (2H, m), 4.59 (1H, brs), 6.13 (1H, s), 6.40 (1H, s), 7.10-7.27 (3H, m), 7.45-7.60 (1H, m), 7.63-7.75 (2H, m), 9.49 (1H, s).

IR (KBr) 3700-2300, 1719, 1647, 1481 cm⁻¹.

Elemental Analysis (C₃₆H₃₉N₂O₉Cl.6H₂O) Cal'd: C, 61.07; H, 6.01; N, 3.96. Found: C, 60.67; H, 5.98; N, 4.36.

### EXAMPLE 165

7-[[[(3R,5S)-7-Chloro-5-(2,3-dimethoxyphenyl)-1-(3-hydroxy-2,2-dimethylpropyl)-2-oxo-1,2,3,5-tetrahydro-5,1-benzoxazepin-3-yl]acetyl]amino]-5-chloro-1-benzofuran-2-carboxylic acid

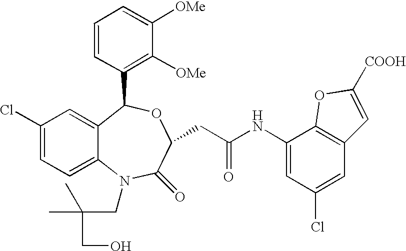

(1) Fuming nitric acid (d=1.52) (5.4 ml, 124.55 mmol) was added dropwise to acetic anhydride (30 ml) cooled to −10° C., and 5-chlorosalicylaldehyde (15 g, 95.80 mmol) was gradually added. After the mixture was stirred at the same temperature for 2 hours, an aqueous saturated sodium bicarbonate solution was added, and extracted with ethyl acetate. The organic layer was washed with water and an aqueous saturated sodium chloride solution, dried with anhydrous sodium sulfate, and concentrated under reduced pressure. The resulting residue (12.9 g) was dissolved in N,N-dimethylformamide (50 ml) under the argon atmosphere, and potassium carbonate (17.7 g, 128.00 mmol) was added. Ethyl bromoacetate (7.8 ml, 70.40 mmol) was added at room temperature, the mixture was stirred for 1 hour, and stirred at 80° C. for 17 hours. Allowing to cool, water was added, and the mixture was extracted with ethyl acetate. The organic layers were combined, and washed with water and an aqueous saturated sodium chloride solution. The organic layer was dried with anhydrous sodium sulfate, and concentrated under reduced pressure. The resulting crude crystals were washed with methanol, dried under reduced pressure to obtain ethyl 5-chloro-7-nitro-1-benzofuran-2-carboxylate (2.3 g, yield 8.9% (2 steps)) as pale yellowish white crystals.

m.p. 111.0-111.5° C.

¹H-NMR (200 MHz, CDCl₃) δ: 1.46 (3H, t, J=7.4 Hz), 4.49 (2H, q, J=7.4 Hz), 7.59 (1H, s), 8.00 (1H, d, J=2.2 Hz), 8.29 (1H, d, J=2.2 Hz).

IR (KBr) 1721, 1572, 1539, 1352, 1318, 1190 cm⁻¹.

Elemental Analysis (C₁₁H₈NO₅Cl) Cal'd: C, 49.00; H, 2.99; N, 5.19. Found: C, 48.91; H, 2.75; N, 5.22.

(2) Ethyl 5-chloro-7-nitro-1-benzofuran-2-carboxylate (0.7 g, 2.60 mmol) obtained in Example 165-(1) was dissolved in ethyl acetate (10 ml), and nitrogen replacement was performed. 10% palladium carbon (70 mg) was placed therein, and hydrogen was introduced. The mixture was stirred at room temperature for 7 hours, the catalyst was filtered, 4N hydrogen chloride-ethyl acetate (0.65 ml) was added, the mixture was stirred at room temperature for 1 hour, the crystals were filtered, and washed with ethyl acetate. Drying under reduced pressure (50° C.) afforded ethyl 7-amino-5-chloro-1-benzofuran-2-carboxylate hydrochloride (0.58 g, yield 80.8%) as white crystals.

m.p. 179.5-180.5° C.

¹H-NMR (200 MHz, DMSO-d₆) δ: 1.33 (3H, t, J=6.8 Hz), 4.36 (2H, q, J=6.8 Hz), 6.72 (1H, d, J=1.8 Hz), 6.96 (1H, d, J=1.8 Hz), 7.61 (1H, s).

IR (KBr) 3600-1900, 1721, 1705, 1574, 1304, 1196 cm⁻¹.

Elemental Analysis (C₁₁H₁₁NO₃Cl₂.4H₂O) Cal'd: C, 46.63; H, 4.20; N, 4.94. Found: C, 46.91; H, 4.29; N, 4.97.

(3) (3R,5S)-1-(3-Acetoxy-2,2-dimethylpropyl)-7-chloro-5-(2,3-dimethoxyphenyl)-1,2,3,5-tetrahydro-2-oxo-4,1-benzoxazepine-3-acetic acid (0.85 g, 1.63 mmol) obtained in Example 1-(1) was dissolved in N,N-dimethylformamide (8.5 ml) under the argon atmosphere. Triethylamine (0.23 ml, 1.66 mmol) and isobutyl chloroformate (0.24 ml, 1.87 mmol) were added under ice-cooling, and the mixture was stirred at the same temperature for 1 hour. Ethyl 7-amino-5-chloro-1-benzofuran-2-carboxylate hydrochloride (0.45 g, 1.63 mmol) obtained in Example 165-(2) was added, and pyridine (0.21 ml, 2.61 mmol) was added dropwise. The mixture was stirred at the same temperature for 2 hours, water was added to the reaction solution, and extracted with ethyl acetate. The organic layer was washed with 1N hydrochloric acid, water and an aqueous saturated sodium chloride solution. The organic layer was dried with anhydrous sodium sulfate, and concentrated under reduced pressure. The resulting residue was purified by silica gel column chromatography (hexane:ethyl acetate=3:2) to obtain ethyl 7-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-5-chloro-1-benzofuran-2-carboxylate (0.98 g, yield 81.0%) as a colorless foam.

[α]_{D}²²=−156.9° (c=0.30, methanol).

¹H-NMR (200 MHz, CDCl₃) δ: 0.96 (3H, s), 1.04 (3H, s), 1.42 (3H, t, J=7.2 Hz), 2.02 (3H, s), 2.96 (1H, dd, J=14.6, 5.8 Hz), 3.18 (1H, dd, J=14.6, 7.6 Hz), 3.56 (1H, d, J=14.2 Hz), 3.62 (3H, s), 3.74 (1H, d, J=11.0 Hz), 3.88 (1H, d, J=11.0 Hz), 3.89, (3H, s), 4.43 (2H, q, J=7.2 Hz), 4.45-4.55 (1H, m), 4.63 (1H, d, J=14.2 Hz), 6.31 (1H, s), 6.66 (1H, s), 6.96 (1H, dd, J=8.0, 1.8 Hz), 7.08 (1H, t, J=8.0 Hz), 7.18 (1H, dd, J=8.0, 1.8 Hz), 7.35 (3H, brs), 7.44 (1H, s), 8.38 (2H, s).

IR (KBr) 3299, 2969, 1738, 1669, 1481, 1244, 1188 cm⁻¹.

Elemental Analysis (C₃₇H₃₈N₂O₁₀Cl₂) Cal'd: C, 59.92; H, 5.16; N, 3.78. Found: C, 59.65; H, 5.02; N, 3.66.

(4) Ethyl 7-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-5-chloro-1-benzofuran-2-carboxylate (0.8 g, 1.08 mmol) obtained in Example 165-(1) was suspended in ethanol (16 ml), a 2N aqueous sodium hydroxide solution (1.62 ml) was added at room temperature, and the mixture was stirred at room temperature for 1 hour. 1N hydrochloric acid was added to the mixture to acidic, the mixture was concentrated under reduced pressure, and the residue was extracted with ethyl acetate. The organic layer was washed with water and an aqueous saturated sodium chloride solution. The organic layer was dried with anhydrous sodium sulfate, and concentrated under reduced pressure. The resulting crude crystals were recrystallized from ethyl acetate (20 ml)-hexane (40 ml) to obtain 7-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-5-chloro-1-benzofuran-2-carboxylic acid (0.74 g, yield quant) as white crystals.

m.p. 179.2-180.2° C.

[α]_{D}²²=−139.8° (c=0.25, methanol).

¹H-NMR (200 MHz, DMSO-d₆) δ: 0.77 (3H, s), 0.86 (3H, s), 2.98-3.20 (4H, m), 3.52 (3H, s), 3.69 (1H, d, J=14.6 Hz), 3.84 (3H, s), 4.29-4.41 (2H, m), 4.56 (1H, brs), 6.12 (1H, s), 6.40 (1H, d, J=2.2 Hz), 7.00-7.16 (3H, m), 7.50-7.60 (2H, m), 7.65 (1H, s), 7.74 (1H, d, J=8.8 Hz), 8.02 (1H, d, J=1.8 Hz), 10.60 (1H, s).

IR (KBr) 3500-2300, 1732, 1705, 1651, 1530, 1483, 1291-1

Elemental Analysis (C₃₃H₃₂N₂O₉Cl₂.AcOEt) Cal'd: C, 58.50; H, 5.31; N, 3.69. Found: C, 58.40; H, 5.33; N, 3.81.

### PREPARATION EXAMPLE

An agent for hyperlipidemia containing the compound of the formula (I) of the present invention as an active ingredient can be prepared, for example, according to the following formulation.

1. Capsule

| | | |
|---|---|---|
| (1) | 3-[3-[[(3R, 5S)-7-chloro-5-(2,3- | 10 mg |
| | dimethoxyphenyl)-1-(3-hydroxy-2,2- | |
| | dimethylpropyl)-2-oxo-1,2,3,5-tetrahydro- | |
| | 4,1-benzoxazepin-3-yl]acetyl]aminophenyl]- | |
| | propionic acid | |
| (2) | Lactose | 90 mg |
| (3) | Microcrystalline cellulose | 70 mg |
| (4) | Magnesium stearate | 10 mg |
| | 1 Capsule | 180 mg |

(1), (2) and (3) and ½ of (4) are kneaded and then granulated. To this is added the remaining (4), and the whole is sealed into a gelatin capsule.

2. Tablet

| | | |
|---|---|---|
| (1) | 3-[3-[[(3R, 5S)-7-chloro-5-(2,3- | 10 mg |
| | dimethoxyphenyl)-1-(3-hydroxy-2,2- | |
| | dimethylpropyl)-2-oxo-1,2,3,5-tetrahydro- | |
| | 4,1-benzoxazepin-3-yl]acetyl]aminophenyl]- | |
| | propionic acid | |
| (2) | Lactose | 35 mg |
| (3) | Corn starch | 150 mg |
| (4) | Microcrystalline cellulose | 30 mg |
| (5) | Magnesium stearate | 5 mg |
| | 1 Tablet | 230 mg |

(1), (2), (3), ⅔ of (4) and ½ of (5) are kneaded and then granulated. To this granule are added the remaining (4) and (5), which is compression-molded into tablets.

3. Injectable

| | | |
|---|---|---|
| (1) | 3-[3-[[(3R, 5S)-7-chloro-5-(2,3- | 10 mg |
| | dimethoxyphenyl)-1-(3-hydroxy-2,2- | |
| | dimethylpropyl)-2-oxo-1,2,3,5-tetrahydro- | |
| | 4,1-benzoxazepin-3-yl]acetyl]aminophenyl]- | |
| | propionic acid | |
| (2) | Inositol | 100 mg |
| (3) | Benzyl alcohol | 20 mg |
| | 1 Ampoule | 130 mg |

(1), (2) and (3) are dissolved in distilled water for injection to a total of 2 ml, which is sealed into an ampoule. All steps are conducted under sterilized conditions.

### TEST EXAMPLE 1

Squalene Synthase Inhibiting Activity

Assay Method

The squalene synthase inhibiting activity was measured using an enzyme solution obtained according to a preparing method described below as follows:

That is, an enzyme solution (protein 0.8 μg) prepared according to the following preparing method was added to a solution containing 5 μM [1-³H] farnesyl pyrophosphate (specific activity 25 μCi/mole), 1 mM NADPH (reduced type nicotinamide adenine dinucleotide phosphate), 5 mM MgCl₂, 6 mM glutathione, 100 mM potassium phosphate buffer (pH 7.4) and a test drug (added as an aqueous solution or DMSO solution)(total amount 50 μl), which was reacted at 37° C. for 45 minutes. 150 μl of a mixed solution of chloroform and methanol (1:2) was added to stop the reaction, and 50 μl of chloroform and 50 μl of a 3N sodium hydroxide solution were added. 50 μl of the chloroform layer (lower layer) containing the reaction product, a main component of which is squalene, and 3 ml of toluene series liquid scintillator were mixed, and the radioactivity thereof was measured by a liquid scintillation counter.

The squalene synthase inhibiting activity was shown by the concentration at which 50% of the radioactivity is incorporated into the chloroform layer (IC₅₀, molar concentration (M)). The results are shown in Table 1.

Preparation of Human Enzyme Solution

Human hepatic cancer cell HepG2 (about 1×10⁹ cells) was grown in Dulbecco's modified Eagle medium (37° C., in the presence of 5%CO₂) containing 10% bovine fetal serum, the resulting cells were suspended in 10 ml of ice-cooled buffer [100 mM potassium phosphate buffer (pH 7.4), 30 mM nicotinamide, 2.5 mM MgCl₂], and ruptured by the ultrasonication (30 seconds, 2 times). The resulting sonicate was centrifuged at 10000× g for 20 minutes (4° C.). The resulting supernatant was centrifuged at 105000× g for 90 minutes (4° C.), then the sediment was suspended in an ice-cooled 100 mM potassium phosphate buffer (pH 7.4), and centrifuged again at 105000× g for 90 minutes (4° C.). This was suspended in an ice-cooled 100 mM potassium phosphate buffer (pH 7.4) (protein concentration about 4 mg/ml), which was used as an enzyme solution.

TABLE 1

| Compound No. | Inhibiting activity |
|---|---|
| (Example No.) | (IC₅₀, 10⁻⁹M) |
| 2 | 54 |
| 18 | 10 |
| 23 | 99 |
| 24 | 170 |
| 26 | 25 |
| 30 | 9.1 |
| 35 | 120 |
| 37 | 94 |
| 53 | 40 |
| 55 | 16 |
| 60 | 50 |
| 61 | 21 |
| 64 | 37 |

As apparent from the above results, the present compounds have the excellent squalene synthesizing inhibiting activity.

The present compounds have the squalene synthase inhibiting activity, the cholesterol lowering activity and the triglyceride lowering activity, are useful as a lipid lowering agent for preventing and/or treating hyperlipidemia and also useful for preventing and/or treating atherosclerosis.

1. A compound represented by the formula [I]: 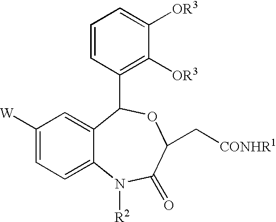
wherein R¹ is optionally substituted 1-carboxyethyl group, optionally substituted carboxy-C₃₋₆ straight alkyl group, optionally substituted C₃₋₆ straight alkyl-sulfonyl group, optionally substituted (carboxy-C₅₋₇ cycloalkyl)-C₁₋₃ alkyl group, or a group represented by the formula: —X¹—X²—Ar—X³—X⁴—COOH (wherein each of X¹ and X⁴ is a bond or optionally substituted C₁₋₄ alkylene group, each of X² and X³ is a bond, —O— or —S—, and Ar is optionally substituted bivalent aromatic group, provided that, when X¹ is a bond, X² is a bond and, when X⁴ is a bond, X³ is a bond), R² is C₃₋₆ alkyl group optionally substituted with alkanoyloxy group and/or hydroxy group, R³ is lower alkyl group, and W is halogen atom, provided that, when R¹ is optionally substituted 1-carboxyethyl group, optionally substituted C₃₋₆ straight alkyl group, 4-carboxycyclohexylmethyl group or 4-carboxymethylphenyl group, R² is C₃₋₆ alkyl group having alkanoyloxy group and/or hydroxy group, or a salt thereof.

2. The compound according to claim 1, wherein R¹ is 3-carboxypropyl group, 1-carboxyethyl group, optionally substituted C₃₋₆ straight alkyl-sulfonyl group, optinally substituted (carboxy-C₅₋₇ cycloalkyl)-C₁₋₃ alkyl group, optionally substituted (carboxyfuryl)-alkyl group, optionally substituted carboxy-C₆₋₁₀ aryl group, (carboxy-C₂₋₃ alkyl) —C₆₋₁₀ aryl group or (carboxy-C₁₋₃ alkyl) —C₇₋₁₄ aralkyl group.

3. The compound according to claim 1, wherein R¹ is optionally substituted (carboxy-C₁₋₄ alkyl)-C₆₋₁₀ aryl group.

4. The compound according to claim 1, wherein R¹ is optionally substituted (carboxy-C₂₋₃ alkyl)-C₆₋₁₀ aryl group.

5. The compound according to claim 1, wherein R¹ is optionally substituted (carboxy-C₂₋₃ alkyl)-phenyl group.

6. The compound according to claim 1, wherein R¹ is optionally substituted (carboxyfuryl)-alkyl group.

7. The compound according to claim 1, wherein R² is C₃₋₆ alkyl group having alkanoyloxy group and/or hydroxy group.

8. The compound according to claim 1, wherein R² is C₃₋₆ alkyl group optionally having 1 to 3 substituents selected from hydroxy group, acetoxy, propionyloxy, t-butoxycarbonyloxy and palmitoyloxy.

9. The compound according to claim 1, wherein R² is 2,2-dimethylpropyl, 3-hydroxy-2,2-dimethylpropyl or 3-acetoxy-2,2-dimethylpropyl.

10. The compound according to claim 1, wherein R³ is methyl group.

11. The compound according to claim 1, wherein W is chlorine atom.

12. The compound according to claim 1, wherein a 3-position is R-configuration and a 5-position is S-configuration.

13. The compound according to claim 1, which is:
(3R,5S)-N-propanesulfonyl-7-chloro-5-(2,3-dimethoxyphenyl)-1-(3-hydroxy-2,2-dimethylpropyl)-1,2,3,5-tetrahydro-4,1-benzoxazepine-3-acetamide, or a salt thereof
(2R)-2-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminopropionic acid, or a salt thereof,
3-[3-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminophenyl]propionic acid, or a salt thereof, or
4-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminobutanoic acid, or a salt thereof.

14. The compound according to claim 1, which is:
trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl-1-cyclohexanecarboxylic acid, or a salt thereof,
trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl-1-cyclohexanecarboxylic acid, or a salt thereof,
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionic acid, or a salt thereof,
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylphenyl]propionic acid, or a salt thereof,
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid, or a salt thereof,
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid, or a salt thereof,
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid, or a salt thereof,
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]propionic acid, or a salt thereof,
4-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxylphenyl]butanoic acid, or a salt thereof,
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid, or a salt thereof, or
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid, or a salt thereof.

15. The compound according to claim 1, which is:
2-[2-[[[(3R,5S)-7-chloro-5-(2,3-dimethoxyphenyl)-1-(3-hydroxypropyl-2,2-dimethylpropyl)-2-oxo-1,2,3,5-tetrahydro-4,1-benzoxazepin-3-yl]acetyl]amino]ethyl]furan-3-carboxylic acid, or a salt thereof,
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid, or a salt thereof, or
3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid, or a salt thereof.

16. A prodrug of a compound represented by the formula [I] 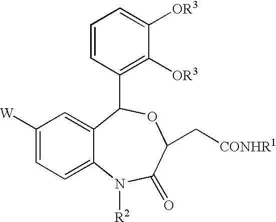
wherein each symbol is as defined in claim, or a salt thereof.

17. A process for producing a compound represented by the formula [I]: 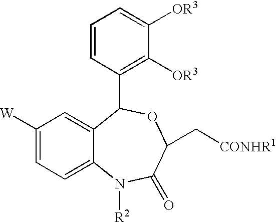
wherein each symbol is as defined in claim 1, or a salt thereof, which comprises reacting a compound represented by the formula [II]: 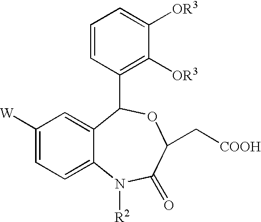
wherein each symbol is as defined in claim 1, or a salt thereof or a reactive derivative of the carboxyl group, with a compound represented by the formula:
wherein each symbol is as defined in claim 1, or a salt thereof.

18. A pharmaceutical composition comprises a compound represented by the formula [I]: 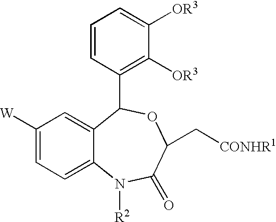
wherein each symbol is as defined in claim 1, a salt thereof or a prodrug thereof.

19. The pharmaceutical composition according to claim 18, which is a squalene synthase inhibitor.

20. The pharmaceutical composition according to claim 18, which is a triglyceride lowering agent.

21. The pharmaceutical composition according to claim 18, which is a lipid lowering agent.

22. The pharmaceutical composition according to claim 18, which is an agent for preventing and/or treating hyperlipidemia.

23. The pharmaceutical composition according to claim 18, which is a high-density lipoproetin cholesterol increasing agent.

24. A method for inhibiting squalene synthase in a mammal in need thereof which comprises administering an effective amount of the compound according to claim 1, or a salt or a prodrug thereof to said mammal.

25. A method for lowering triglycerides in a mammal in need thereof which comprises administering an effective amount of the compound according to claim 1, or a salt or a prodrug thereof to said mammal.

26. A method for lowering lipid in a mammal in need thereof which comprises administering an effective amount of the compound according to claim 1, or a salt or a prodrug thereof to said mammal.

27. A method for preventing and/or treating hyperlipidemia of a mammal in need thereof which comprises administering an effective amount of the compound according to claim 1, or a salt or a prodrug thereof to said mammal.

28. A method for increasing high-density lipoprotein-cholesterol in a mammal in need thereof which comprises administering an effective amount of the compound according to claim 1, or a salt or a prodrug thereof to said mammal.

29. Use of the compound according to claim 1, or a salt or a prodrug thereof for manufacturing a squalene synthase inhibior.

30. Use of the compound according to claim 1, or a salt or a prodrug thereof for manufacturing a triglyceride lowering agent.

31. Use of the compound according to claim 1, or a salt or a prodrug thereof for manufacturing a lipid lowering agent.

32. Use of the compound according to claim 1, or a salt or a prodrug thereof for manufacturing an agent for preventing and/or treating hyperlipidemia.

33. Use of the compound according to claim 1, or a salt or a prodrug thereof for manufacturing a high-density lipoprotein-cholesterol increasing agent.
